SEQUENCE LISTING (939 sequences)

SEQ ID NO: 1 - protein, 10 aa, Artificial Sequence
  source: /note=“Description of Artificial Sequence: Synthetic peptide”
GYTFTNYGIN

SEQ ID NO: 2 - protein, 17 aa, Artificial Sequence
  source: /note=“Description of Artificial Sequence: Synthetic peptide”
YIYIGNDYTEYNERFKG

SEQ ID NO: 3 - protein, 14 aa, Artificial Sequence
  source: /note=“Description of Artificial Sequence: Synthetic peptide”
LYYGSSLYSYAMDY

SEQ ID NO: 4 - protein, 5 aa, Artificial Sequence
  source: /note=“Description of Artificial Sequence: Synthetic peptide”
NYGIN

SEQ ID NO: 5 - protein, 7 aa, Artificial Sequence
  source: /note=“Description of Artificial Sequence: Synthetic peptide”
GYTFTNY

SEQ ID NO: 6 - protein, 6 aa, Artificial Sequence
  source: /note=“Description of Artificial Sequence: Synthetic peptide”
YIGNDY

SEQ ID NO: 7 - protein, 8 aa, Artificial Sequence
  source: /note=“Description of Artificial Sequence: Synthetic peptide”
GYTFTNYG

SEQ ID NO: 8 - protein, 5 aa, Artificial Sequence
  source: /note=“Description of Artificial Sequence: Synthetic peptide”
IYIGN

SEQ ID NO: 9 - protein, 16 aa, Artificial Sequence
  source: /note=“Description of Artificial Sequence: Synthetic peptide”
ARLYYGSSLYSYAMDY

SEQ ID NO: 10 - protein, 123 aa, Artificial Sequence
  source: /note=“Description of Artificial Sequence: Synthetic polypeptide”
QVQLVQSGAEVKKPGASVKVSCKASGYTFTNYGINWVRQAPGQRLEWMGYIYIGNDYTEYNERFKGRVTITSDTSASTAYMELSSLRSEDTAVYYCARLYYGSSLYSYAMDYWGQGTTVTVSS

SEQ ID NO: 11 - DNA, 369 bp, Artificial Sequence
  source: /note=“Description of Artificial Sequence: Synthetic polynucleotide”
caagttcagttggttcagtctggcgccgaagtgaagaaacctggcgcctctgtgaaggtgtcctgcaaggcttctggctacacctttaccaactacggcatcaactgggtccgacaggctcctggccagagattggagtggatgggctacatctacatcggcaacgactacaccgagtacaacgagcggttcaagggcagagtgaccatcacctctgacacctctgcctccaccgcctacatggaactgtccagcctgagatctgaggacaccgccgtgtactactgcgccaggctgtactatggctcctccctgtacagctatgccatggactactggggacagggcacaaccgtgacagtgagctcc

SEQ ID NO: 12 - protein, 453 aa, Artificial Sequence
  source: /note=“Description of Artificial Sequence: Synthetic polypeptide”
QVQLVQSGAEVKKPGASVKVSCKASGYTFTNYGINWVRQAPGQRLEWMGYIYIGNDYTEYNERFKGRVTITSDTSASTAYMELSSLRSEDTAVYYCARLYYGSSLYSYAMDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 13 - DNA, 1359 bp, Artificial Sequence
  source: /note=“Description of Artificial Sequence: Synthetic polynucleotide”
caagttcagttggttcagtctggcgccgaagtgaagaaacctggcgcctctgtgaaggtgtcctgcaaggcttctggctacacctttaccaactacggcatcaactgggtccgacaggctcctggccagagattggagtggatgggctacatctacatcggcaacgactacaccgagtacaacgagcggttcaagggcagagtgaccatcacctctgacacctctgcctccaccgcctacatggaactgtccagcctgagatctgaggacaccgccgtgtactactgcgccaggctgtactatggctcctccctgtacagctatgccatggactactggggacagggcacaaccgtgacagtgagctccgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 14 - protein, 16 aa, Artificial Sequence
  source: /note=“Description of Artificial Sequence: Synthetic peptide”
RSSKSLLHSSGNTYLY

SEQ ID NO: 15 - protein, 7 aa, Artificial Sequence
  source: /note=“Description of Artificial Sequence: Synthetic peptide”
RMSNLAS

SEQ ID NO: 16 - protein, 9 aa, Artificial Sequence
  source: /note=“Description of Artificial Sequence: Synthetic peptide”
MQHLEYPYT

SEQ ID NO: 17 - protein, 12 aa, Artificial Sequence
  source: /note=“Description of Artificial Sequence: Synthetic peptide”
SKSLLHSSGNTY

SEQ ID NO: 18 - protein, 3 aa, Artificial Sequence
  source: /note=“Description of Artificial Sequence: Synthetic peptide”
RMS

SEQ ID NO: 19 - protein, 6 aa, Artificial Sequence
  source: /note=“Description of Artificial Sequence: Synthetic peptide”
HLEYPY

SEQ ID NO: 20 - protein, 11 aa, Artificial Sequence
  source: /note=“Description of Artificial Sequence: Synthetic peptide”
KSLLHSSGNTY

SEQ ID NO: 21 - protein, 112 aa, Artificial Sequence
  source: /note=“Description of Artificial Sequence: Synthetic polypeptide”
DIVMTQSPLSLPVTPGEPASISCRSSKSLLHSSGNTYLYWFLQKPGQSPQLLISRMSNLASGVPDRFSGSGSGTDFTLKISRVEAEDVGVYYCMQHLEYPYTFGGGTKVEIK

SEQ ID NO: 22 - DNA, 336 bp, Artificial Sequence
  source: /note=“Description of Artificial Sequence: Synthetic polynucleotide”
gacattgtgatgacccagtctccactgagcctgcctgtgacacctggcgagcctgcttccatctcctgccggtcctctaagtccctgctgcactcttccggcaatacctacctgtactggttcctgcagaagcccggccagtctcctcagctgctgatctccagaatgtccaacctggcctctggcgtgcccgacagattttctggctctggatctggcaccgacttcaccctgaagatctctagagtggaagccgaggacgtgggcgtgtactactgtatgcagcacctggaatacccctacaccttcggcggaggcaccaaggtggaaatcaag

SEQ ID NO: 23 - protein, 219 aa, Artificial Sequence
  source: /note=“Description of Artificial Sequence: Synthetic polypeptide”
DIVMTQSPLSLPVTPGEPASISCRSSKSLLHSSGNTYLYWFLQKPGQSPQLLISRMSNLASGVPDRFSGSGSGTDFTLKISRVEAEDVGVYYCMQHLEYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 657 bp, Artificial Sequence
  source: /note=“Description of Artificial Sequence: Synthetic polynucleotide”
gacattgtgatgacccagtctccactgagcctgcctgtgacacctggcgagcctgcttccatctcctgccggtcctctaagtccctgctgcactcttccggcaatacctacctgtactggttcctgcagaagcccggccagtctcctcagctgctgatctccagaatgtccaacctggcctctggcgtgcccgacagattttctggctctggatctggcaccgacttcaccctgaagatctctagagtggaagccgaggacgtgggcgtgtactactgtatgcagcacctggaatacccctacaccttcggcggaggcaccaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 25 - protein, 12 aa, Artificial Sequence
  source: /note=“Description of Artificial Sequence: Synthetic peptide”
GFSLSTGGMSVS

SEQ ID NO: 26 - protein, 16 aa, Artificial Sequence
  source: /note=“Description of Artificial Sequence: Synthetic peptide”
LIDWDDDKYYSTSLKT

SEQ ID NO: 27 - protein, 9 aa, Artificial Sequence
  source: /note=“Description of Artificial Sequence: Synthetic peptide”
AHSGSYFDF

SEQ ID NO: 28 - protein, 7 aa, Artificial Sequence
  source: /note=“Description of Artificial Sequence: Synthetic peptide”
TGGMSVS

SEQ ID NO: 29 - protein, 9 aa, Artificial Sequence
  source: /note=“Description of Artificial Sequence: Synthetic peptide”
GFSLSTGGM

SEQ ID NO: 30 - protein, 5 aa, Artificial Sequence
  source: /note=“Description of Artificial Sequence: Synthetic peptide”
DWDDD

SEQ ID NO: 31 - protein, 10 aa, Artificial Sequence
  source: /note=“Description of Artificial Sequence: Synthetic peptide”
GFSLSTGGMS

SEQ ID NO: 32 - protein, 7 aa, Artificial Sequence
  source: /note=“Description of Artificial Sequence: Synthetic peptide”
IDWDDDK

SEQ ID NO: 33 - protein, 11 aa, Artificial Sequence
  source: /note=“Description of Artificial Sequence: Synthetic peptide”
ARAHSGSYFDF

SEQ ID NO: 34 - protein, 119 aa, Artificial Sequence
  source: /note=“Description of Artificial Sequence: Synthetic polypeptide”
QVTLRESGPALVKPTQTLTLTCTFSGFSLSTGGMSVSWIRQPPGKALEWLALIDWDDDKYYSTSLKTRLTISKDTSKNQLVLTMTNMDPVDTATYYCARAHSGSYFDFWGQGTLVTVSS

SEQ ID NO: 35 - DNA, 357 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactggtggaatgagtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctca

SEQ ID NO: 36 - protein, 449 aa, Artificial Sequence
  source: /note=“Description of Artificial Sequence: Synthetic polypeptide”
QVTLRESGPALVKPTQTLTLTCTFSGFSLSTGGMSVSWIRQPPGKALEWLALIDWDDDKYYSTSLKTRLTISKDTSKNQLVLTMTNMDPVDTATYYCARAHSGSYFDFWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 37 - DNA, 1347 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactggtggaatgagtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 38 - protein, 11 aa, Artificial Sequence
  source: /note=“Description of Artificial Sequence: Synthetic peptide”
RASQRISNWLA

SEQ ID NO: 39 - protein, 7 aa, Artificial Sequence
  source: /note=“Description of Artificial Sequence: Synthetic peptide”
KASSLES

SEQ ID NO: 40 - protein, 8 aa, Artificial Sequence
  source: /note=“Description of Artificial Sequence: Synthetic peptide”
QQFSSYWT

SEQ ID NO: 41 - protein, 7 aa, Artificial Sequence
  source: /note=“Description of Artificial Sequence: Synthetic peptide”
SQRISNW

SEQ ID NO: 42 - protein, 3 aa, Artificial Sequence
  source: /note=“Description of Artificial Sequence: Synthetic peptide”
KAS

SEQ ID NO: 43 - protein, 5 aa, Artificial Sequence
  source: /note=“Description of Artificial Sequence: Synthetic peptide”
FSSYW

SEQ ID NO: 44 - protein, 6 aa, Artificial Sequence
  source: /note=“Description of Artificial Sequence: Synthetic peptide”
QRISNW

SEQ ID NO: 45 - protein, 106 aa, Artificial Sequence
  source: /note=“Description of Artificial Sequence: Synthetic polypeptide”
DIQMTQSPSTLSASVGDRVTITCRASQRISNWLAWYQQKPGKAPKLLIYKASSLESGVPSRFSGSGSGTEFTLTISSLQPDDFATYYCQQFSSYWTFGQGTKVEIK

SEQ ID NO: 46 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttagtagttattggacgttcggccaagggaccaaggtggaaatcaaa

SEQ ID NO: 47 - protein, 213 aa, Artificial Sequence
  source: /note=“Description of Artificial Sequence: Synthetic polypeptide”
DIQMTQSPSTLSASVGDRVTITCRASQRISNWLAWYQQKPGKAPKLLIYKASSLESGVPSRFSGSGSGTEFTLTISSLQPDDFATYYCQQFSSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttagtagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 49 - protein, 12 aa, Artificial Sequence
  source: /note=“Description of Artificial Sequence: Synthetic peptide”
GFSLSTSGISVS

SEQ ID NO: 50 - protein, 12 aa, Artificial Sequence
  source: /note=“Description of Artificial Sequence: Synthetic peptide”
TPSGSYGRYFDL

SEQ ID NO: 51 - protein, 7 aa, Artificial Sequence
  source: /note=“Description of Artificial Sequence: Synthetic peptide”
TSGISVS

SEQ ID NO: 52 - protein, 9 aa, Artificial Sequence
  source: /note=“Description of Artificial Sequence: Synthetic peptide”
GFSLSTSGI

SEQ ID NO: 53 - protein, 10 aa, Artificial Sequence
  source: /note=“Description of Artificial Sequence: Synthetic peptide”
GFSLSTSGIS

SEQ ID NO: 54 - protein, 14 aa, Artificial Sequence
  source: /note=“Description of Artificial Sequence: Synthetic peptide”
ARTPSGSYGRYFDL

SEQ ID NO: 55 - protein, 122 aa, Artificial Sequence
  source: /note=“Description of Artificial Sequence: Synthetic polypeptide”
QVTLRESGPALVKPTQTLTLTCTFSGFSLSTSGISVSWIRQPPGKALEWLALIDWDDDKYYSTSLKTRLTISKDTSKNQVVLTMTNMDPVDTATYYCARTPSGSYGRYFDLWGRGTLVTVSS

SEQ ID NO: 56 - DNA, 366 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcacaagtggaatatctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggacccctagtgggagctatgggcgatacttcgatctctggggccgtggcaccctggtcactgtctcctca

SEQ ID NO: 57 - protein, 452 aa, Artificial Sequence
  source: /note=“Description of Artificial Sequence: Synthetic polypeptide”
QVTLRESGPALVKPTQTLTLTCTFSGFSLSTSGISVSWIRQPPGKALEWLALIDWDDDKYYSTSLKTRLTISKDTSKNQVVLTMTNMDPVDTATYYCARTPSGSYGRYFDLWGRGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 58 - DNA, 1356 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcacaagtggaatatctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggacccctagtgggagctatgggcgatacttcgatctctggggccgtggcaccctggtcactgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 59 - protein, 11 aa, Artificial Sequence
  source: /note=“Description of Artificial Sequence: Synthetic peptide”
RASQSISNWLA

SEQ ID NO: 60 - protein, 8 aa, Artificial Sequence
  source: /note=“Description of Artificial Sequence: Synthetic peptide”
QQYNAYWT

SEQ ID NO: 61 - protein, 7 aa, Artificial Sequence
  source: /note=“Description of Artificial Sequence: Synthetic peptide”
SQSISNW

SEQ ID NO: 62 - protein, 5 aa, Artificial Sequence
  source: /note=“Description of Artificial Sequence: Synthetic peptide”
YNAYW

SEQ ID NO: 63 - protein, 6 aa, Artificial Sequence
  source: /note=“Description of Artificial Sequence: Synthetic peptide”
QSISNW

SEQ ID NO: 64 - protein, 106 aa, Artificial Sequence
  source: /note=“Description of Artificial Sequence: Synthetic polypeptide”
DIQLTQSPSTLSASVGDRVTITCRASQSISNWLAWYQQKPGKAPKLLIYKASSLESGVPSRFTGSGSGTEFTLTISSLQPDDFATYYCQQYNAYWTFGQGTKVEIK

SEQ ID NO: 65 - DNA, 318 bp, Artificial Sequence
  source: /note=“Description of Artificial Sequence: Synthetic polynucleotide”
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatgcctattggacgttcggccaagggaccaaggtggaaatcaaa

SEQ ID NO: 66 - protein, 213 aa, Artificial Sequence
  source: /note=“Description of Artificial Sequence: Synthetic polypeptide”
DIQLTQSPSTLSASVGDRVTITCRASQSISNWLAWYQQKPGKAPKLLIYKASSLESGVPSRFTGSGSGTEFTLTISSLQPDDFATYYCQQYNAYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 639 bp, Artificial Sequence
  source: /note=“Description of Artificial Sequence: Synthetic polynucleotide”
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatgcctattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 68 - protein, 8 aa, Artificial Sequence
  source: /note=“Description of Artificial Sequence: Synthetic peptide”
QQFQSYWT

SEQ ID NO: 69 - protein, 5 aa, Artificial Sequence
  source: /note=“Description of Artificial Sequence: Synthetic peptide”
FQSYW

SEQ ID NO: 70 - protein, 106 aa, Artificial Sequence
  source: /note=“Description of Artificial Sequence: Synthetic polypeptide”
DIQMTQSPSTLSASVGDRVTITCRASQRISNWLAWYQQKPGKAPKLLIYKASSLESGVPSRFSGSGSGTEFTLTISSLQPDDFATYYCQQFQSYWTFGQGTKVEIK

SEQ ID NO: 71 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttcagagttattggacgttcggccaagggaccaaggtggaaatcaaa

SEQ ID NO: 72 - protein, 213 aa, Artificial Sequence
  source: /note=“Description of Artificial Sequence: Synthetic polypeptide”
DIQMTQSPSTLSASVGDRVTITCRASQRISNWLAWYQQKPGKAPKLLIYKASSLESGVPSRFSGSGSGTEFTLTISSLQPDDFATYYCQQFQSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttcagagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 74 - protein, 12 aa, Artificial Sequence
  source: /note=“Description of Artificial Sequence: Synthetic peptide”
GFSLSTSGVSVS

SEQ ID NO: 75 - protein, 7 aa, Artificial Sequence
  source: /note=“Description of Artificial Sequence: Synthetic peptide”
TSGVSVS

SEQ ID NO: 76 - protein, 9 aa, Artificial Sequence
  source: /note=“Description of Artificial Sequence: Synthetic peptide”
GFSLSTSGV

SEQ ID NO: 77 - protein, 10 aa, Artificial Sequence
  source: /note=“Description of Artificial Sequence: Synthetic peptide”
GFSLSTSGVS

SEQ ID NO: 78 - protein, 122 aa, Artificial Sequence
  source: /note=“Description of Artificial Sequence: Synthetic polypeptide”
QVTLRESGPALVKPTQTLTLTCTFSGFSLSTSGVSVSWIRQPPGKALEWLALIDWDDDKYYSTSLKTRLTISKDTSKNQVVLTMTNMDPVDTATYYCARTPSGSYGRYFDLWGRGTLVTVSS

SEQ ID NO: 79 - DNA, 366 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactagtggagtgtctgtgagttggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtactattgtgcacggacccctagtgggagctacgggcgatacttcgatctctggggccgtggcaccctggtcactgtctcctca

SEQ ID NO: 80 - protein, 452 aa, Artificial Sequence
  source: /note=“Description of Artificial Sequence: Synthetic polypeptide”
QVTLRESGPALVKPTQTLTLTCTFSGFSLSTSGVSVSWIRQPPGKALEWLALIDWDDDKYYSTSLKTRLTISKDTSKNQVVLTMTNMDPVDTATYYCARTPSGSYGRYFDLWGRGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 81 - DNA, 1356 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactagtggagtgtctgtgagttggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtactattgtgcacggacccctagtgggagctacgggcgatacttcgatctctggggccgtggcaccctggtcactgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 82 - protein, 8 aa, Artificial Sequence
  source: /note=“Description of Artificial Sequence: Synthetic peptide”
QQYQSYWT

SEQ ID NO: 83 - protein, 5 aa, Artificial Sequence
  source: /note=“Description of Artificial Sequence: Synthetic peptide”
YQSYW

SEQ ID NO: 84 - protein, 106 aa, Artificial Sequence
  source: /note=“Description of Artificial Sequence: Synthetic polypeptide”
DIQMTQSPSTLSASVGDRVTITCRASQSISNWLAWYQQKPGKAPKLLIYKASSLESGVPSRFSGSGSGTEFTLTISSLQPDDFATYYCQQYQSYWTFGQGTKVEIK

SEQ ID NO: 85 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaa

SEQ ID NO: 86 - protein, 213 aa, Artificial Sequence
  source: /note=“Description of Artificial Sequence: Synthetic polypeptide”
DIQMTQSPSTLSASVGDRVTITCRASQSISNWLAWYQQKPGKAPKLLIYKASSLESGVPSRFSGSGSGTEFTLTISSLQPDDFATYYCQQYQSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 88 - protein, 12 aa, Artificial Sequence
  source: /note=“Description of Artificial Sequence: Synthetic peptide”
GFSLSTSGVSVT

SEQ ID NO: 89 - protein, 7 aa, Artificial Sequence
  source: /note=“Description of Artificial Sequence: Synthetic peptide”
TSGVSVT

SEQ ID NO: 90 - protein, 122 aa, Artificial Sequence
  source: /note=“Description of Artificial Sequence: Synthetic polypeptide”
QVTLRESGPALVKPTQTLTLTCTFSGFSLSTSGVSVTWIRQPPGKALEWLALIDWDDDKYYSTSLKTRLAISKDTSKNQVVLTMTNMDPVDTATYYCARTPSGSYGRYFDLWGRGTLVTVSS

SEQ ID NO: 91 - DNA, 366 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactagtggagtgtctgtgacctggatccgtcagcccccagggaaggccctggagtggcttgcactcattgattgggatgatgataaatactacagcacatctctgaagaccaggctcgccatctccaaggacacctccaaaaaccaggtggtccttacaatgaccaacatggaccctgtggacacagccacgtattattgtgcacggacccctagtgggagctacgggcgatacttcgatctctggggccgtggcaccctggtcactgtctcctca

SEQ ID NO: 92 - protein, 452 aa, Artificial Sequence
  source: /note=“Description of Artificial Sequence: Synthetic polypeptide”
QVTLRESGPALVKPTQTLTLTCTFSGFSLSTSGVSVTWIRQPPGKALEWLALIDWDDDKYYSTSLKTRLAISKDTSKNQVVLTMTNMDPVDTATYYCARTPSGSYGRYFDLWGRGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 93 - DNA, 1356 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactagtggagtgtctgtgacctggatccgtcagcccccagggaaggccctggagtggcttgcactcattgattgggatgatgataaatactacagcacatctctgaagaccaggctcgccatctccaaggacacctccaaaaaccaggtggtccttacaatgaccaacatggaccctgtggacacagccacgtattattgtgcacggacccctagtgggagctacgggcgatacttcgatctctggggccgtggcaccctggtcactgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 94 - protein, 11 aa, Artificial Sequence
  source: /note=“Description of Artificial Sequence: Synthetic peptide”
RASQSISTWLA

SEQ ID NO: 95 - protein, 7 aa, Artificial Sequence
  source: /note=“Description of Artificial Sequence: Synthetic peptide”
EASSLES

SEQ ID NO: 96 - protein, 7 aa, Artificial Sequence
  source: /note=“Description of Artificial Sequence: Synthetic peptide”
SQSISTW

SEQ ID NO: 97 - protein, 3 aa, Artificial Sequence
  source: /note=“Description of Artificial Sequence: Synthetic peptide”
EAS

SEQ ID NO: 98 - protein, 6 aa, Artificial Sequence
  source: /note=“Description of Artificial Sequence: Synthetic peptide”
QSISTW

SEQ ID NO: 99 - protein, 106 aa, Artificial Sequence
  source: /note=“Description of Artificial Sequence: Synthetic polypeptide”
DIQMTQSPSTLSASVGDRVTITCRASQSISTWLAWYQQKPGKAPKLLIYEASSLESGVPSRFSGSGSGTEFTLTISSLQPDDFATYYCQQYQSYWTFGQGTKVEIK

SEQ ID NO: 100 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaa

SEQ ID NO: 101 - protein, 213 aa, Artificial Sequence
  source: /note=“Description of Artificial Sequence: Synthetic polypeptide”
DIQMTQSPSTLSASVGDRVTITCRASQSISTWLAWYQQKPGKAPKLLIYEASSLESGVPSRFSGSGSGTEFTLTISSLQPDDFATYYCQQYQSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 103 - protein, 123 aa, Artificial Sequence
  source: /note=“Description of Artificial Sequence: Synthetic polypeptide”
EVQLQQSGAELVRPGSSVKMSCKTSGYTFTNYGINWVKQRPGQGLEWIGYIYIGNDYTEYNERFKGKATLTSDTSSSTAHIQLNSLTSEDSAIYFCARLYYGSSLYSYAMDYWGQGTSVTVSS

SEQ ID NO: 104 - DNA, 369 bp, Artificial Sequence
  source: /note=“Description of Artificial Sequence: Synthetic polynucleotide”
gaggttcagctgcagcagtctggagctgagttggtgaggcctgggtcctcagtgaagatgtcctgcaagacttctggatatacattcacaaactacggtataaactgggtgaagcagaggcctggacagggcctggaatggattggatatatttatattggaaatgattatactgagtacaatgagaggttcaagggcaaggccacactgacttcagacacatcctccagcacagcccacatacaactcaacagcctgacatctgaggactctgcaatctatttctgtgcaagactttactacggtagtagcctctattcttatgctatggactactggggtcaaggaacctctgtcacagtctcctct

SEQ ID NO: 105 - protein, 453 aa, Artificial Sequence
  source: /note=“Description of Artificial Sequence: Synthetic polypeptide”
EVQLQQSGAELVRPGSSVKMSCKTSGYTFTNYGINWVKQRPGQGLEWIGYIYIGNDYTEYNERFKGKATLTSDTSSSTAHIQLNSLTSEDSAIYFCARLYYGSSLYSYAMDYWGQGTSVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 106 - DNA, 1359 bp, Artificial Sequence
  source: /note=“Description of Artificial Sequence: Synthetic polynucleotide”
gaggttcagctgcagcagtctggagctgagttggtgaggcctgggtcctcagtgaagatgtcctgcaagacttctggatatacattcacaaactacggtataaactgggtgaagcagaggcctggacagggcctggaatggattggatatatttatattggaaatgattatactgagtacaatgagaggttcaagggcaaggccacactgacttcagacacatcctccagcacagcccacatacaactcaacagcctgacatctgaggactctgcaatctatttctgtgcaagactttactacggtagtagcctctattcttatgctatggactactggggtcaaggaacctctgtcacagtctcctctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 107 - protein, 112 aa, Artificial Sequence
  source: /note=“Description of Artificial Sequence: Synthetic polypeptide”
DIVMTQAAPSVSVTPGESVSISCRSSKSLLHSSGNTYLYWFLQRPGQSPQLLISRMSNLASGVPDRFSGSGSGTAFTLRISRVEAEDVGVYYCMQHLEYPYTFGGGTKLELK

SEQ ID NO: 108 - DNA, 336 bp, Artificial Sequence
  source: /note=“Description of Artificial Sequence: Synthetic polynucleotide”
gatattgtgatgactcaggctgcaccctctgtatctgtcactcctggagagtcagtatccatctcctgcaggtctagtaagagtctcctccatagtagtggcaacacttacttgtattggttcctgcagaggccaggccagtctcctcagctcctgatatctcggatgtccaaccttgcctcaggagtcccagacaggttcagtggcagtgggtcaggaactgctttcacactgagaatcagtagagtggaggctgaggatgtgggtgtttattattgtatgcaacatctagaatatccgtacacgttcggaggggggaccaagctggagctaaaa

SEQ ID NO: 109 - protein, 219 aa, Artificial Sequence
  source: /note=“Description of Artificial Sequence: Synthetic polypeptide”
DIVMTQAAPSVSVTPGESVSISCRSSKSLLHSSGNTYLYWFLQRPGQSPQLLISRMSNLASGVPDRFSGSGSGTAFTLRISRVEAEDVGVYYCMQHLEYPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - DNA, 657 bp, Artificial Sequence
  source: /note=“Description of Artificial Sequence: Synthetic polynucleotide”
gatattgtgatgactcaggctgcaccctctgtatctgtcactcctggagagtcagtatccatctcctgcaggtctagtaagagtctcctccatagtagtggcaacacttacttgtattggttcctgcagaggccaggccagtctcctcagctcctgatatctcggatgtccaaccttgcctcaggagtcccagacaggttcagtggcagtgggtcaggaactgctttcacactgagaatcagtagagtggaggctgaggatgtgggtgtttattattgtatgcaacatctagaatatccgtacacgttcggaggggggaccaagctggagctaaaacgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 111 - protein, 12 aa, Artificial Sequence
  source: /note=“Description of Artificial Sequence: Synthetic peptide”
GFSLSTGGMCVS

SEQ ID NO: 112 - protein, 7 aa, Artificial Sequence
  source: /note=“Description of Artificial Sequence: Synthetic peptide”
TGGMCVS

SEQ ID NO: 113 - protein, 10 aa, Artificial Sequence
  source: /note=“Description of Artificial Sequence: Synthetic peptide”
GFSLSTGGMC

SEQ ID NO: 114 - protein, 119 aa, Artificial Sequence
  source: /note=“Description of Artificial Sequence: Synthetic polypeptide”
QVTLRESGPALVKPTQTLTLTCTFSGFSLSTGGMCVSWIRQPPGKALEWLALIDWDDDKYYSTSLKTRLTISKDTSKNQLVLTMTNMDPVDTATYYCARAHSGSYFDFWGQGTLVTVSS

SEQ ID NO: 115 - DNA, 357 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactggtggaatgtgtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctca

SEQ ID NO: 116 - protein, 449 aa, Artificial Sequence
  source: /note=“Description of Artificial Sequence: Synthetic polypeptide”
QVTLRESGPALVKPTQTLTLTCTFSGFSLSTGGMCVSWIRQPPGKALEWLALIDWDDDKYYSTSLKTRLTISKDTSKNQLVLTMTNMDPVDTATYYCARAHSGSYFDFWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 117 - DNA, 1347 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcactcagcactggtggaatgtgtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 118 - protein, 8 aa, Artificial Sequence
  source: /note=“Description of Artificial Sequence: Synthetic peptide”
QQFNSYWT

SEQ ID NO: 119 - protein, 5 aa, Artificial Sequence
  source: /note=“Description of Artificial Sequence: Synthetic peptide”
FNSYW

SEQ ID NO: 120 - protein, 106 aa, Artificial Sequence
  source: /note=“Description of Artificial Sequence: Synthetic polypeptide”
DIQMTQSPSTLSASVGDRVTITCRASQRISNWLAWYQQKPGKAPKLLIYKASSLESGVPSRFSGSGSGTEFTLTISSLQPDDFATYYCQQFNSYWTFGQGTKVEIK

SEQ ID NO: 121 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaa

SEQ ID NO: 122 - protein, 213 aa, Artificial Sequence
  source: /note=“Description of Artificial Sequence: Synthetic polypeptide”
DIQMTQSPSTLSASVGDRVTITCRASQRISNWLAWYQQKPGKAPKLLIYKASSLESGVPSRFSGSGSGTEFTLTISSLQPDDFATYYCQQFNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 124 - protein, 8 aa, Artificial Sequence
  source: /note=“Description of Artificial Sequence: Synthetic peptide”
QQYNSYWT

SEQ ID NO: 125 - protein, 5 aa, Artificial Sequence
  source: /note=“Description of Artificial Sequence: Synthetic peptide”
YNSYW

SEQ ID NO: 126 - protein, 106 aa, Artificial Sequence
  source: /note=“Description of Artificial Sequence: Synthetic polypeptide”
DIQLTQSPSTLSASVGDRVTITCRASQSISNWLAWYQQKPGKAPKLLIYKASSLESGVPSRFTGSGSGTEFTLTISSLQPDDFATYYCQQYNSYWTFGQGTKVEIK

SEQ ID NO: 127 - DNA, 318 bp, Artificial Sequence
  source: /note=“Description of Artificial Sequence: Synthetic polynucleotide”
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatagttattggacgttcggccaagggaccaaggtggaaatcaaa

SEQ ID NO: 128 - protein, 213 aa, Artificial Sequence
  source: /note=“Description of Artificial Sequence: Synthetic polypeptide”
DIQLTQSPSTLSASVGDRVTITCRASQSISNWLAWYQQKPGKAPKLLIYKASSLESGVPSRFTGSGSGTEFTLTISSLQPDDFATYYCQQYNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - DNA, 639 bp, Artificial Sequence
  source: /note=“Description of Artificial Sequence: Synthetic polynucleotide”
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 130 - protein, 106 aa, Artificial Sequence
  source: /note=“Description of Artificial Sequence: Synthetic polypeptide”
DIQMTQSPSTLSASVGDRVTITCRASQSISNWLAWYQQKPGKAPKLLIYKASSLESGVPSRFSGSGSGTEFTLTISSLQPDDFATYYCQQYNSYWTFGQGTKVEIK

SEQ ID NO: 131 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaa

SEQ ID NO: 132 - protein, 213 aa, Artificial Sequence
  source: /note=“Description of Artificial Sequence: Synthetic polypeptide”
DIQMTQSPSTLSASVGDRVTITCRASQSISNWLAWYQQKPGKAPKLLIYKASSLESGVPSRFSGSGSGTEFTLTISSLQPDDFATYYCQQYNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 134 - protein, 106 aa, Artificial Sequence
  source: /note=“Description of Artificial Sequence: Synthetic polypeptide”
DIQMTQSPSTLSASVGDRVTITCRASQSISTWLAWYQQKPGKAPKLLIYEASSLESGVPSRFSGSGSGTEFTLTISSLQPDDFATYYCQQYNSYWTFGQGTKVEIK

SEQ ID NO: 135 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaa

SEQ ID NO: 136 - protein, 213 aa, Artificial Sequence
  source: /note=“Description of Artificial Sequence: Synthetic polypeptide”
DIQMTQSPSTLSASVGDRVTITCRASQSISTWLAWYQQKPGKAPKLLIYEASSLESGVPSRFSGSGSGTEFTLTISSLQPDDFATYYCQQYNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 138 - protein, 12 aa, Artificial Sequence
  source: /note=“Description of Artificial Sequence: Synthetic peptide”
GFSPSTSGMSVS

SEQ ID NO: 139 - protein, 16 aa, Artificial Sequence
  source: /note=“Description of Artificial Sequence: Synthetic peptide”
LIDWDDDKYFSTSLKT

SEQ ID NO: 140 - protein, 9 aa, Artificial Sequence
  source: /note=“Description of Artificial Sequence: Synthetic peptide”
AHSGSYFDY

SEQ ID NO: 141 - protein, 7 aa, Artificial Sequence
  source: /note=“Description of Artificial Sequence: Synthetic peptide”
TSGMSVS

SEQ ID NO: 142 - protein, 9 aa, Artificial Sequence
  source: /note=“Description of Artificial Sequence: Synthetic peptide”
GFSPSTSGM

SEQ ID NO: 143 - protein, 10 aa, Artificial Sequence
  source: /note=“Description of Artificial Sequence: Synthetic peptide”
GFSPSTSGMS

SEQ ID NO: 144 - protein, 11 aa, Artificial Sequence
  source: /note=“Description of Artificial Sequence: Synthetic peptide”
ARAHSGSYFDY

SEQ ID NO: 145 - protein, 119 aa, Artificial Sequence
  source: /note=“Description of Artificial Sequence: Synthetic polypeptide”
QVTLRESGPALVKPTQTLTLTCTFSGFSPSTSGMSVSWIRQPPGKALEWLALIDWDDDKYFSTSLKTRLTISKDTSKNQVVLTMTNMDPVDTATYYCARAHSGSYFDYWGQGTLVTVSS

SEQ ID NO: 146 - DNA, 357 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcacccagcactagtggaatgtctgtgagctggatccgtcagcccccagggaaggccctggagtggcttgcacttattgattgggatgatgataaatactttagcacatctctgaagaccaggctcaccatctccaaggacacctccaaaaaccaggtggtccttacaatgaccaacatggaccctgtagacacagccacgtattattgtgcacgggcccatagtgggagctactttgactactggggccagggaaccctggtcaccgtctcctca

SEQ ID NO: 147 - protein, 449 aa, Artificial Sequence
  source: /note=“Description of Artificial Sequence: Synthetic polypeptide”
QVTLRESGPALVKPTQTLTLTCTFSGFSPSTSGMSVSWIRQPPGKALEWLALIDWDDDKYFSTSLKTRLTISKDTSKNQVVLTMTNMDPVDTATYYCARAHSGSYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 148 - DNA, 1347 bp, Artificial Sequence
  source: /note=“Description of Artificial Sequence: Synthetic polynucleotide”
caggtcaccttgagggagtctggtcctgcgctggtgaaacccacacagaccctcacactgacctgcaccttctctgggttctcacccagcactagtggaatgtctgtgagctggatccgtcagcccccagggaaggccctggagtggcttgcacttattgattgggatgatgataaatactttagcacatctctgaagaccaggctcaccatctccaaggacacctccaaaaaccaggtggtccttacaatgaccaacatggaccctgtagacacagccacgtattattgtgcacgggcccatagtgggagctactttgactactggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 149 - protein, 106 aa, Artificial Sequence
  source: /note=“Description of Artificial Sequence: Synthetic polypeptide”
DIQMTQSPSTLSASVGDRVTITCRASQSISNWLAWYQQKPGKAPKLLIYKASSLESGVPSRFSGSGSGTEFTLTISSLQPDDFATYYCQQFNSYWTFGQGTKVEIK

SEQ ID NO: 150 - DNA, 318 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaa

SEQ ID NO: 151 - protein, 213 aa, Artificial Sequence
  source: /note=“Description of Artificial Sequence: Synthetic polypeptide”
DIQMTQSPSTLSASVGDRVTITCRASQSISNWLAWYQQKPGKAPKLLIYKASSLESGVPSRFSGSGSGTEFTLTISSLQPDDFATYYCQQFNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - DNA, 639 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 153 - protein, 10 aa, Artificial Sequence
  source: /note=“Description of Artificial Sequence: Synthetic peptide”
GGSISSYYWN

SEQ ID NO: 154 - protein, 16 aa, Artificial Sequence
  source: /note=“Description of Artificial Sequence: Synthetic peptide”
RIYTSGSTNYNPSLKS

SEQ ID NO: 155 - protein, 13 aa, Artificial Sequence
  source: /note=“Description of Artificial Sequence: Synthetic peptide”
DSGGFYYYYGMDV

SEQ ID NO: 156 - protein, 5 aa, Artificial Sequence
  source: /note=“Description of Artificial Sequence: Synthetic peptide”
SYYWN

SEQ ID NO: 157 - protein, 7 aa, Artificial Sequence
  source: /note=“Description of Artificial Sequence: Synthetic peptide”
GGSISSY

SEQ ID NO: 158 - protein, 5 aa, Artificial Sequence
  source: /note=“Description of Artificial Sequence: Synthetic peptide”
YTSGS

SEQ ID NO: 159 - protein, 8 aa, Artificial Sequence
  source: /note=“Description of Artificial Sequence: Synthetic peptide”
GGSISSYY

SEQ ID NO: 160 - protein, 7 aa, Artificial Sequence
  source: /note=“Description of Artificial Sequence: Synthetic peptide”
IYTSGST

SEQ ID NO: 161 - protein, 15 aa, Artificial Sequence
  source: /note=“Description of Artificial Sequence: Synthetic peptide”
ARDSGGFYYYYGMDV

SEQ ID NO: 162 - protein, 121 aa, Artificial Sequence
  source: /note=“Description of Artificial Sequence: Synthetic polypeptide”
QVQLQESGPGLVKPSETLSLTCAVSGGSISSYYWNLIRQPAGKGLEWIGRIYTSGSTNYNPSLKSRVTMSVDTSKNQFSLKLSSVTAADTAVYYCARDSGGFYYYYGMDVWGQGTTVTVSS

SEQ ID NO: 163 - DNA, 363 bp, Artificial Sequence
  source: /note=“Description of Artificial Sequence: Synthetic polynucleotide”
caggtgcagctgcaggagtcgggcccaggactggtgaagccttcggagaccctgtccctcacctgcgctgtctctggtggctccatcagtagttactactggaacttaatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagactccggggggttctactactactacggtatggacgtctggggccaagggaccacggtcaccgtctcctca

SEQ ID NO: 164 - protein, 451 aa, Artificial Sequence
  source: /note=“Description of Artificial Sequence: Synthetic polypeptide”
QVQLQESGPGLVKPSETLSLTCAVSGGSISSYYWNLIRQPAGKGLEWIGRIYTSGSTNYNPSLKSRVTMSVDTSKNQFSLKLSSVTAADTAVYYCARDSGGFYYYYGMDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 165 - DNA, 1353 bp, Artificial Sequence
  source: /note=“Description of Artificial Sequence: Synthetic polynucleotide”
caggtgcagctgcaggagtcgggcccaggactggtgaagccttcggagaccctgtccctcacctgcgctgtctctggtggctccatcagtagttactactggaacttaatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagactccggggggttctactactactacggtatggacgtctggggccaagggaccacggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 166 - protein, 11 aa, Artificial Sequence
  source: /note=“Description of Artificial Sequence: Synthetic peptide”
RASQGISSYLA

SEQ ID NO: 167 - protein, 7 aa, Artificial Sequence
  source: /note=“Description of Artificial Sequence: Synthetic peptide”
AASTLQG

SEQ ID NO: 168 - protein, 9 aa, Artificial Sequence
  source: /note=“Description of Artificial Sequence: Synthetic peptide”
QQLNSYPWT

SEQ ID NO: 169 - protein, 7 aa, Artificial Sequence
  source: /note=“Description of Artificial Sequence: Synthetic peptide”
SQGISSY

SEQ ID NO: 170 - protein, 3 aa, Artificial Sequence
  source: /note=“Description of Artificial Sequence: Synthetic peptide”
AAS

SEQ ID NO: 171 - protein, 6 aa, Artificial Sequence
  source: /note=“Description of Artificial Sequence: Synthetic peptide”
LNSYPW

SEQ ID NO: 172 - protein, 6 aa, Artificial Sequence
  source: /note=“Description of Artificial Sequence: Synthetic peptide”
QGISSY

SEQ ID NO: 173 - protein, 10 aa, Artificial Sequence
  source: /note=“Description of Artificial Sequence: Synthetic peptide”
AASTLQGGVP

SEQ ID NO: 174 - protein, 107 aa, Artificial Sequence
  source: /note=“Description of Artificial Sequence: Synthetic polypeptide”
DIQLTQSPSFLSASVGDRVTITCRASQGISSYLAWYQQKPGKAPKLLIYAASTLQGGVPSRFSGSGSGTEFTLTISSLQPEDFATYHCQQLNSYPWTFGQGTKVEIK

SEQ ID NO: 175 - DNA, 321 bp, Artificial Sequence
  source: /note=“Description of Artificial Sequence: Synthetic polynucleotide”
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccaccttgcaaggtggggtcccatcaaggttcagcggcagtggatctgggacagaattcactctcacaatcagcagcctgcagcctgaagattttgcaacttatcactgtcaacagcttaatagttacccgtggacgttcggccaagggaccaaggtggaaatcaaa

SEQ ID NO: 176 - protein, 214 aa, Artificial Sequence
  source: /note=“Description of Artificial Sequence: Synthetic polypeptide”
DIQLTQSPSFLSASVGDRVTITCRASQGISSYLAWYQQKPGKAPKLLIYAASTLQGGVPSRFSGSGSGTEFTLTISSLQPEDFATYHCQQLNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - DNA, 642 bp, Artificial Sequence
  source: /note=“Description of Artificial Sequence: Synthetic polynucleotide”
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccaccttgcaaggtggggtcccatcaaggttcagcggcagtggatctgggacagaattcactctcacaatcagcagcctgcagcctgaagattttgcaacttatcactgtcaacagcttaatagttacccgtggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 178 - protein, 10 aa, Artificial Sequence
  source: /note=“Description of Artificial Sequence: Synthetic peptide”
GFTFNNYWMN

SEQ ID NO: 179 - protein, 17 aa, Artificial Sequence
  source: /note=“Description of Artificial Sequence: Synthetic peptide”
NIRQDGSEKYYVDSVKG

SEQ ID NO: 180 - protein, 17 aa, Artificial Sequence
  source: /note=“Description of Artificial Sequence: Synthetic peptide”
ERAYCSTTSCPDYSNDY

SEQ ID NO: 181 - protein, 5 aa, Artificial Sequence
  source: /note=“Description of Artificial Sequence: Synthetic peptide”
NYWMN

SEQ ID NO: 182 - protein, 7 aa, Artificial Sequence
  source: /note=“Description of Artificial Sequence: Synthetic peptide”
GFTFNNY

SEQ ID NO: 183 - protein, 6 aa, Artificial Sequence
  source: /note=“Description of Artificial Sequence: Synthetic peptide”
RQDGSE

SEQ ID NO: 184 - protein, 8 aa, Artificial Sequence
  source: /note=“Description of Artificial Sequence: Synthetic peptide”
GFTFNNYW

SEQ ID NO: 185 - protein, 8 aa, Artificial Sequence
  source: /note=“Description of Artificial Sequence: Synthetic peptide”
IRQDGSEK

SEQ ID NO: 186 - protein, 19 aa, Artificial Sequence
  source: /note=“Description of Artificial Sequence: Synthetic peptide”
ARERAYCSTTSCPDYSNDY

SEQ ID NO: 187 - protein, 126 aa, Artificial Sequence
  source: /note=“Description of Artificial Sequence: Synthetic polypeptide”
EVQLVESGGGLVQPGGSLRLSCAASGFTFNNYWMNWVRQAPGKGLEWVANIRQDGSEKYYVDSVKGRFTISRDNAKNSLFLQMNSLRAEDTAVYYCARERAYCSTTSCPDYSNDYWGQGTLVTVSS

SEQ ID NO: 188 - DNA, 378 bp, Artificial Sequence
  source: /note=“Description of Artificial Sequence: Synthetic polynucleotide”
gaggtgcagctggtggagtctgggggaggcttggtccagcctggggggtccctgaggctctcctgtgcagcctctggattcacctttaataactattggatgaactgggtccgccaggctccagggaaggggctggagtgggtggccaacataagacaagatggaagtgaaaaatactatgtggactctgtgaagggccgattcaccatctccagagacaacgccaagaactcactgtttctacaaatgaacagcctgagagccgaggacacggctgtatattactgtgcgagagagagggcctattgtagtactaccagctgccctgactacagtaatgactactggggccagggaaccctggtcaccgtctcctca

SEQ ID NO: 189 - protein, 456 aa, Artificial Sequence
  source: /note=“Description of Artificial Sequence: Synthetic polypeptide”
EVQLVESGGGLVQPGGSLRLSCAASGFTFNNYWMNWVRQAPGKGLEWVANIRQDGSEKYYVDSVKGRFTISRDNAKNSLFLQMNSLRAEDTAVYYCARERAYCSTTSCPDYSNDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 190 - DNA, 1368 bp, Artificial Sequence
  source: /note=“Description of Artificial Sequence: Synthetic polynucleotide”
gaggtgcagctggtggagtctgggggaggcttggtccagcctggggggtccctgaggctctcctgtgcagcctctggattcacctttaataactattggatgaactgggtccgccaggctccagggaaggggctggagtgggtggccaacataagacaagatggaagtgaaaaatactatgtggactctgtgaagggccgattcaccatctccagagacaacgccaagaactcactgtttctacaaatgaacagcctgagagccgaggacacggctgtatattactgtgcgagagagagggcctattgtagtactaccagctgccctgactacagtaatgactactggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 191 - protein, 11 aa, Artificial Sequence
  source: /note=“Description of Artificial Sequence: Synthetic peptide”
RASQTINNNLA

SEQ ID NO: 192 - protein, 7 aa, Artificial Sequence
  source: /note=“Description of Artificial Sequence: Synthetic peptide”
GASTGAT

SEQ ID NO: 193 - protein, 11 aa, Artificial Sequence
  source: /note=“Description of Artificial Sequence: Synthetic peptide”
QQYNNWPRGLT

SEQ ID NO: 194 - protein, 7 aa, Artificial Sequence
  source: /note=“Description of Artificial Sequence: Synthetic peptide”
SQTINNN

SEQ ID NO: 195 - protein, 3 aa, Artificial Sequence
  source: /note=“Description of Artificial Sequence: Synthetic peptide”
GAS

SEQ ID NO: 196 - protein, 8 aa, Artificial Sequence
  source: /note=“Description of Artificial Sequence: Synthetic peptide”
YNNWPRGL

SEQ ID NO: 197 - protein, 6 aa, Artificial Sequence
  source: /note=“Description of Artificial Sequence: Synthetic peptide”
QTINNN

SEQ ID NO: 198 - protein, 10 aa, Artificial Sequence
  source: /note=“Description of Artificial Sequence: Synthetic peptide”
GASTGATGIP

SEQ ID NO: 199 - protein, 109 aa, Artificial Sequence
  source: /note=“Description of Artificial Sequence: Synthetic polypeptide”
EIVMTQSPATLSVSPGERATLSCRASQTINNNLAWFQQKPGQTPRLLIYGASTGATGIPARFSGSGSGTEFTLTISSLQSEDFAVYYCQQYNNWPRGLTFGGGTKVEIK

SEQ ID NO: 200 - DNA, 327 bp, Artificial Sequence
  source: /note=“Description of Artificial Sequence: Synthetic polynucleotide”
gaaatagtgatgacgcagtctccagccaccctgtctgtgtctccaggggaaagagccaccctctcctgcagggccagtcagactattaacaacaacttagcctggttccagcagaaacctggccagactcccaggctcctcatctatggtgcatccaccggggccactggtatcccagccaggttcagtggcagtgggtctgggacagagttcactctcaccatcagcagcctgcagtctgaagattttgcagtttattactgtcagcagtataataactggcctcgaggactcactttcggcggagggaccaaggtggagatcaaa

SEQ ID NO: 201 - protein, 216 aa, Artificial Sequence
  source: /note=“Description of Artificial Sequence: Synthetic polypeptide”
EIVMTQSPATLSVSPGERATLSCRASQTINNNLAWFQQKPGQTPRLLIYGASTGATGIPARFSGSGSGTEFTLTISSLQSEDFAVYYCQQYNNWPRGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - DNA, 648 bp, Artificial Sequence
  source: /note=“Description of Artificial Sequence: Synthetic polynucleotide”
gaaatagtgatgacgcagtctccagccaccctgtctgtgtctccaggggaaagagccaccctctcctgcagggccagtcagactattaacaacaacttagcctggttccagcagaaacctggccagactcccaggctcctcatctatggtgcatccaccggggccactggtatcccagccaggttcagtggcagtgggtctgggacagagttcactctcaccatcagcagcctgcagtctgaagattttgcagtttattactgtcagcagtataataactggcctcgaggactcactttcggcggagggaccaaggtggagatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 203 - protein, 10 aa, Artificial Sequence
  source: /note=“Description of Artificial Sequence: Synthetic peptide”
GYTFTGYYIN

SEQ ID NO: 204 - protein, 17 aa, Artificial Sequence
  source: /note=“Description of Artificial Sequence: Synthetic peptide”
WINPNSGDTNYAQKFQG

SEQ ID NO: 205 - protein, 11 aa, Artificial Sequence
  source: /note=“Description of Artificial Sequence: Synthetic peptide”
ENSGYGKLFDY

SEQ ID NO: 206 - protein, 5 aa, Artificial Sequence
  source: /note=“Description of Artificial Sequence: Synthetic peptide”
GYYIN

SEQ ID NO: 207 - protein, 7 aa, Artificial Sequence
  source: /note=“Description of Artificial Sequence: Synthetic peptide”
GYTFTGY

SEQ ID NO: 208 - protein, 6 aa, Artificial Sequence
  source: /note=“Description of Artificial Sequence: Synthetic peptide”
NPNSGD

SEQ ID NO: 209 - protein, 8 aa, Artificial Sequence
  source: /note=“Description of Artificial Sequence: Synthetic peptide”
GYTFTGYY

SEQ ID NO: 210 - protein, 8 aa, Artificial Sequence
  source: /note=“Description of Artificial Sequence: Synthetic peptide”
INPNSGDT

SEQ ID NO: 211 - protein, 13 aa, Artificial Sequence
  source: /note=“Description of Artificial Sequence: Synthetic peptide”
ARENSGYGKLFDY

SEQ ID NO: 212 - protein, 120 aa, Artificial Sequence
  source: /note=“Description of Artificial Sequence: Synthetic polypeptide”
QVQLVQSGAEVKKPGASVKVSCKASGYTFTGYYINWVRQAPGQGLEWMGWINPNSGDTNYAQKFQGRVTMTRDPSISTAYMELSRLRSDDTAVYYCARENSGYGKLFDYWGQGTLVTVSS

SEQ ID NO: 213 - DNA, 360 bp, Artificial Sequence
  source: /note=“Description of Artificial Sequence: Synthetic polynucleotide”
caggtgcagctggtgcagtctggggctgaggtgaagaagcctggggcctcagtgaaggtctcctgcaaggcttctggatacaccttcaccggctactatataaattgggtgcgacaggcccctggacaagggcttgagtggatgggatggatcaaccctaacagtggtgacacaaactatgcacagaagtttcagggcagggtcaccatgaccagggacccgtccatcagcacagcctacatggagctgagcaggctgagatctgacgacacggccgtgtattactgtgcgagagagaatagtggctacgggaagctttttgactactggggccagggaaccctggtcaccgtctcctca

SEQ ID NO: 214 - protein, 450 aa, Artificial Sequence
  source: /note=“Description of Artificial Sequence: Synthetic polypeptide”
QVQLVQSGAEVKKPGASVKVSCKASGYTFTGYYINWVRQAPGQGLEWMGWINPNSGDTNYAQKFQGRVTMTRDPSISTAYMELSRLRSDDTAVYYCARENSGYGKLFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 215 - DNA, 1350 bp, Artificial Sequence
  source: /note=“Description of Artificial Sequence: Synthetic polynucleotide”
caggtgcagctggtgcagtctggggctgaggtgaagaagcctggggcctcagtgaaggtctcctgcaaggcttctggatacaccttcaccggctactatataaattgggtgcgacaggcccctggacaagggcttgagtggatgggatggatcaaccctaacagtggtgacacaaactatgcacagaagtttcagggcagggtcaccatgaccagggacccgtccatcagcacagcctacatggagctgagcaggctgagatctgacgacacggccgtgtattactgtgcgagagagaatagtggctacgggaagctttttgactactggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 216 - protein, 16 aa, Artificial Sequence
  source: /note=“Description of Artificial Sequence: Synthetic peptide”
RSSQSLLHSNGYNYLD

SEQ ID NO: 217 - protein, 7 aa, Artificial Sequence
  source: /note=“Description of Artificial Sequence: Synthetic peptide”
LGSNRAS

SEQ ID NO: 218 - protein, 9 aa, Artificial Sequence
  source: /note=“Description of Artificial Sequence: Synthetic peptide”
MQALQTPYT

SEQ ID NO: 219 - protein, 12 aa, Artificial Sequence
  source: /note=“Description of Artificial Sequence: Synthetic peptide”
SQSLLHSNGYNY

SEQ ID NO: 220 - protein, 3 aa, Artificial Sequence
  source: /note=“Description of Artificial Sequence: Synthetic peptide”
LGS

SEQ ID NO: 221 - protein, 6 aa, Artificial Sequence
  source: /note=“Description of Artificial Sequence: Synthetic peptide”
ALQTPY

SEQ ID NO: 222 - protein, 11 aa, Artificial Sequence
  source: /note=“Description of Artificial Sequence: Synthetic peptide”
QSLLHSNGYNY

SEQ ID NO: 223 - protein, 112 aa, Artificial Sequence
  source: /note=“Description of Artificial Sequence: Synthetic polypeptide”
DIVMTQSPLSLPGTPGEPASISCRSSQSLLHSNGYNYLDWYLQKPGQSPQFLIYLGSNRASGVPDRFSGSGSGTDFTLKISRVEAEDVGVYYCMQALQTPYTFGQGTKLEIK

SEQ ID NO: 224 - DNA, 336 bp, Artificial Sequence
  source: /note=“Description of Artificial Sequence: Synthetic polynucleotide”
gatattgtgatgactcagtctccactctccctgcccggcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacacttttggccaggggaccaagctggagatcaaa

SEQ ID NO: 225 - protein, 219 aa, Artificial Sequence
  source: /note=“Description of Artificial Sequence: Synthetic polypeptide”
DIVMTQSPLSLPGTPGEPASISCRSSQSLLHSNGYNYLDWYLQKPGQSPQFLIYLGSNRASGVPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - DNA, 657 bp, Artificial Sequence
  source: /note=“Description of Artificial Sequence: Synthetic polynucleotide”
gatattgtgatgactcagtctccactctccctgcccggcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacacttttggccaggggaccaagctggagatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 227 - protein, 10 aa, Artificial Sequence
  source: /note=“Description of Artificial Sequence: Synthetic peptide”
GGSISSYYWT

SEQ ID NO: 228 - protein, 16 aa, Artificial Sequence
  source: /note=“Description of Artificial Sequence: Synthetic peptide”
RVFTSESTNYNPSLKN

SEQ ID NO: 229 - protein, 11 aa, Artificial Sequence
  source: /note=“Description of Artificial Sequence: Synthetic peptide”
DRGTYLGGFDP

SEQ ID NO: 230 - protein, 5 aa, Artificial Sequence
  source: /note=“Description of Artificial Sequence: Synthetic peptide”
SYYWT

SEQ ID NO: 231 - protein, 5 aa, Artificial Sequence
  source: /note=“Description of Artificial Sequence: Synthetic peptide”
FTSES

SEQ ID NO: 232 - protein, 7 aa, Artificial Sequence
  source: /note=“Description of Artificial Sequence: Synthetic peptide”
VFTSEST

SEQ ID NO: 233 - protein, 13 aa, Artificial Sequence
  source: /note=“Description of Artificial Sequence: Synthetic peptide”
ARDRGTYLGGFDP

SEQ ID NO: 234 - protein, 119 aa, Artificial Sequence
  source: /note=“Description of Artificial Sequence: Synthetic polypeptide”
QVQLQESGPGLVKPSETLSLTCTVSGGSISSYYWTWIRQPAGKGLEWIGRVFTSESTNYNPSLKNRVTMSVDTSKNQFSLRLNSVTAADTAMYYCARDRGTYLGGFDPWGQGTLVTVSS

SEQ ID NO: 235 - DNA, 357 bp, Artificial Sequence
  source: /note=“Description of Artificial Sequence: Synthetic polynucleotide”
caggtgcagctgcaggagtcgggcccaggactggtgaagccttcggagaccctgtccctcacctgcactgtctctggtggctccatcagtagttactactggacctggatccggcagcccgccgggaagggactggagtggattgggcgtgtctttaccagtgagagcaccaactacaacccctccctcaagaatcgagtcaccatgtcagtagacacgtccaagaaccagttctccctgaggctgaattctgtgaccgccgcggacacggccatgtattactgtgcgagagaccgggggacctacctaggggggttcgacccctggggccagggaaccctggtcaccgtctcctca

SEQ ID NO: 236 - protein, 449 aa, Artificial Sequence
  source: /note=“Description of Artificial Sequence: Synthetic polypeptide”
QVQLQESGPGLVKPSETLSLTCTVSGGSISSYYWTWIRQPAGKGLEWIGRVFTSESTNYNPSLKNRVTMSVDTSKNQFSLRLNSVTAADTAMYYCARDRGTYLGGFDPWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 237 - DNA, 1347 bp, Artificial Sequence
  source: /note=“Description of Artificial Sequence: Synthetic polynucleotide”
caggtgcagctgcaggagtcgggcccaggactggtgaagccttcggagaccctgtccctcacctgcactgtctctggtggctccatcagtagttactactggacctggatccggcagcccgccgggaagggactggagtggattgggcgtgtctttaccagtgagagcaccaactacaacccctccctcaagaatcgagtcaccatgtcagtagacacgtccaagaaccagttctccctgaggctgaattctgtgaccgccgcggacacggccatgtattactgtgcgagagaccgggggacctacctaggggggttcgacccctggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 238 - protein, 9 aa, Artificial Sequence
  source: /note=“Description of Artificial Sequence: Synthetic peptide”
IQALQTPFT

SEQ ID NO: 239 - protein, 6 aa, Artificial Sequence
  source: /note=“Description of Artificial Sequence: Synthetic peptide”
ALQTPF

SEQ ID NO: 240 - protein, 112 aa, Artificial Sequence
  source: /note=“Description of Artificial Sequence: Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCIQALQTPFTFGQGTKLEIK

SEQ ID NO: 241 - DNA, 336 bp, Artificial Sequence
  source: /note=“Description of Artificial Sequence: Synthetic polynucleotide”
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatacaagctctacaaactccgttcacttttggccaggggaccaaactggagatcaaa

SEQ ID NO: 242 - protein, 219 aa, Artificial Sequence
  source: /note=“Description of Artificial Sequence: Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCIQALQTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - DNA, 657 bp, Artificial Sequence
  source: /note=“Description of Artificial Sequence: Synthetic polynucleotide”
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatacaagctctacaaactccgttcacttttggccaggggaccaaactggagatcaaacgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 244 - protein, 12 aa, Artificial Sequence
  source: /note=“Description of Artificial Sequence: Synthetic peptide”
GFSLSTSGMSVS

SEQ ID NO: 245 - protein, 9 aa, Artificial Sequence
  source: /note=“Description of Artificial Sequence: Synthetic peptide”
MALRHAFDA

SEQ ID NO: 246 - protein, 9 aa, Artificial Sequence
  source: /note=“Description of Artificial Sequence: Synthetic peptide”
GFSLSTSGM

SEQ ID NO: 247 - protein, 10 aa, Artificial Sequence
  source: /note=“Description of Artificial Sequence: Synthetic peptide”
GFSLSTSGMS

SEQ ID NO: 248 - protein, 11 aa, Artificial Sequence
  source: /note=“Description of Artificial Sequence: Synthetic peptide”
ARMALRHAFDA

SEQ ID NO: 249 - protein, 119 aa, Artificial Sequence
  source: /note=“Description of Artificial Sequence: Synthetic polypeptide”
QVTLRESGPALVKPTQTLTLTCTFSGFSLSTSGMSVSWIRQPPGKALEWLALIDWDDDKYYSTSLKTRLTISKDTSKNQVVLTMTNMDPVDTATYYCARMALRHAFDAWGQGTMVTVSS

SEQ ID NO: 250 - DNA, 357 bp, Artificial Sequence
  source: /note=“Description of Artificial Sequence: Synthetic polynucleotide”
caggtcaccttgagggagtctggtcctgcgctggtgaagcccacacagaccctcacactgacctgcaccttctctgggttctcactcagcactagtggaatgtctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggatggcactacgtcatgcttttgatgcctggggccaagggacaatggtcaccgtctcttca

SEQ ID NO: 251 - protein, 449 aa, Artificial Sequence
  source: /note=“Description of Artificial Sequence: Synthetic polypeptide”
QVTLRESGPALVKPTQTLTLTCTFSGFSLSTSGMSVSWIRQPPGKALEWLALIDWDDDKYYSTSLKTRLTISKDTSKNQVVLTMTNMDPVDTATYYCARMALRHAFDAWGQGTMVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 252 - DNA, 1347 bp, Artificial Sequence
  source: /note=“Description of Artificial Sequence: Synthetic polynucleotide”
caggtcaccttgagggagtctggtcctgcgctggtgaagcccacacagaccctcacactgacctgcaccttctctgggttctcactcagcactagtggaatgtctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggatggcactacgtcatgcttttgatgcctggggccaagggacaatggtcaccgtctcttcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 253 - protein, 14 aa, Artificial Sequence
  source: /note=“Description of Artificial Sequence: Synthetic peptide”
TGSSSNIGAGYDVH

SEQ ID NO: 254 - protein, 7 aa, Artificial Sequence
  source: /note=“Description of Artificial Sequence: Synthetic peptide”
VNSNRPS

SEQ ID NO: 255 - protein, 11 aa, Artificial Sequence
  source: /note=“Description of Artificial Sequence: Synthetic peptide”
QSYDSSLSGWV

SEQ ID NO: 256 - protein, 10 aa, Artificial Sequence
  source: /note=“Description of Artificial Sequence: Synthetic peptide”
SSSNIGAGYD

SEQ ID NO: 257 - protein, 3 aa, Artificial Sequence
  source: /note=“Description of Artificial Sequence: Synthetic peptide”
VNS

SEQ ID NO: 258 - protein, 8 aa, Artificial Sequence
  source: /note=“Description of Artificial Sequence: Synthetic peptide”
YDSSLSGW

SEQ ID NO: 259 - protein, 9 aa, Artificial Sequence
  source: /note=“Description of Artificial Sequence: Synthetic peptide”
SSNIGAGYD

SEQ ID NO: 260 - protein, 111 aa, Artificial Sequence
  source: /note=“Description of Artificial Sequence: Synthetic polypeptide”
QSVLTQPPSVSGAPGQRVTISCTGSSSNIGAGYDVHWYQQLPGTAPKLLIYVNSNRPSGVPDRFSGSKSGTSASLAITGLQAEDEADYYCQSYDSSLSGWVFGGGTKLTVL

SEQ ID NO: 261 - DNA, 333 bp, Artificial Sequence
  source: /note=“Description of Artificial Sequence: Synthetic polynucleotide”
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatgttaacagcaatcggccctcaggggtccctgaccgattctctggctccaagtctggcacctcagcctccctggccatcactgggctccaggctgaggatgaggctgattattactgccagtcctatgacagcagcctgagtggttgggtgttcggcggagggaccaagttgaccgtccta

SEQ ID NO: 262 - protein, 217 aa, Artificial Sequence
  source: /note=“Description of Artificial Sequence: Synthetic polypeptide”
QSVLTQPPSVSGAPGQRVTISCTGSSSNIGAGYDVHWYQQLPGTAPKLLIYVNSNRPSGVPDRFSGSKSGTSASLAITGLQAEDEADYYCQSYDSSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 263 - DNA, 651 bp, Artificial Sequence
  source: /note=“Description of Artificial Sequence: Synthetic polynucleotide”
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatgttaacagcaatcggccctcaggggtccctgaccgattctctggctccaagtctggcacctcagcctccctggccatcactgggctccaggctgaggatgaggctgattattactgccagtcctatgacagcagcctgagtggttgggtgttcggcggagggaccaagttgaccgtcctaggtcagcccaaggctgccccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 264 - protein, 10 aa, Artificial Sequence
  source: /note=“Description of Artificial Sequence: Synthetic peptide”
GFSLTNYGVH

SEQ ID NO: 265 - protein, 16 aa, Artificial Sequence
  source: /note=“Description of Artificial Sequence: Synthetic peptide”
VIWRGESTDYNAAFMS

SEQ ID NO: 266 - protein, 11 aa, Artificial Sequence
  source: /note=“Description of Artificial Sequence: Synthetic peptide”
NGGSSGWYFDV

SEQ ID NO: 267 - protein, 5 aa, Artificial Sequence
  source: /note=“Description of Artificial Sequence: Synthetic peptide”
NYGVH

SEQ ID NO: 268 - protein, 7 aa, Artificial Sequence
  source: /note=“Description of Artificial Sequence: Synthetic peptide”
GFSLTNY

SEQ ID NO: 269 - protein, 5 aa, Artificial Sequence
  source: /note=“Description of Artificial Sequence: Synthetic peptide”
WRGES

SEQ ID NO: 270 - protein, 8 aa, Artificial Sequence
  source: /note=“Description of Artificial Sequence: Synthetic peptide”
GFSLTNYG

SEQ ID NO: 271 - protein, 7 aa, Artificial Sequence
  source: /note=“Description of Artificial Sequence: Synthetic peptide”
IWRGEST

SEQ ID NO: 272 - protein, 13 aa, Artificial Sequence
  source: /note=“Description of Artificial Sequence: Synthetic peptide”
ARNGGSSGWYFDV

SEQ ID NO: 273 - protein, 119 aa, Artificial Sequence
  source: /note=“Description of Artificial Sequence: Synthetic polypeptide”
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNGGSSGWYFDVWGQGTTVTVSS

SEQ ID NO: 274 - DNA, 357 bp, Artificial Sequence
  source: /note=“Description of Artificial Sequence: Synthetic polynucleotide”
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacggcggatcctccggctggtactttgatgtgtggggccagggcaccaccgtgacagttagttct

SEQ ID NO: 275 - protein, 449 aa, Artificial Sequence
  source: /note=“Description of Artificial Sequence: Synthetic polypeptide”
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNGGSSGWYFDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 276 - DNA, 1347 bp, Artificial Sequence
  source: /note=“Description of Artificial Sequence: Synthetic polynucleotide”
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacggcggatcctccggctggtactttgatgtgtggggccagggcaccaccgtgacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 277 - protein, 11 aa, Artificial Sequence
  source: /note=“Description of Artificial Sequence: Synthetic peptide”
KASQDVTSAVA

SEQ ID NO: 278 - protein, 7 aa, Artificial Sequence
  source: /note=“Description of Artificial Sequence: Synthetic peptide”
WTSTRHT

SEQ ID NO: 279 - protein, 9 aa, Artificial Sequence
  source: /note=“Description of Artificial Sequence: Synthetic peptide”
QQHYTTPLT

SEQ ID NO: 280 - protein, 7 aa, Artificial Sequence
  source: /note=“Description of Artificial Sequence: Synthetic peptide”
SQDVTSA

SEQ ID NO: 281 - protein, 3 aa, Artificial Sequence
  source: /note=“Description of Artificial Sequence: Synthetic peptide”
WTS

SEQ ID NO: 282 - protein, 6 aa, Artificial Sequence
  source: /note=“Description of Artificial Sequence: Synthetic peptide”
HYTTPL

SEQ ID NO: 283 - protein, 6 aa, Artificial Sequence
  source: /note=“Description of Artificial Sequence: Synthetic peptide”
QDVTSA

SEQ ID NO: 284 - protein, 107 aa, Artificial Sequence
  source: /note=“Description of Artificial Sequence: Synthetic polypeptide”
DIQLTQSPSFLSASVGDRVTITCKASQDVTSAVAWYQQKPGKAPKLLIYWTSTRHTGVPSRFSGSGSGTEYTLTISSLQPEDFATYYCQQHYTTPLTFGQGTKLEIK

SEQ ID NO: 285 - DNA, 321 bp, Artificial Sequence
  source: /note=“Description of Artificial Sequence: Synthetic polynucleotide”
gatattcagctgacccagtctcctagcttcctgtccgcttctgtgggcgacagagtgaccatcacatgcaaggcctctcaggacgtgacctctgccgtggcttggtatcagcagaagcctggcaaggcccctaagctgctgatctactggacctccaccagacacaccggcgtgccctctagattttccggctctggctctggcaccgagtataccctgacaatctccagcctgcagcctgaggacttcgccacctactactgccagcagcactacaccacacctctgacctttggccagggcaccaagctggaaatcaag

SEQ ID NO: 286 - protein, 214 aa, Artificial Sequence
  source: /note=“Description of Artificial Sequence: Synthetic polypeptide”
DIQLTQSPSFLSASVGDRVTITCKASQDVTSAVAWYQQKPGKAPKLLIYWTSTRHTGVPSRFSGSGSGTEYTLTISSLQPEDFATYYCQQHYTTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - DNA, 642 bp, Artificial Sequence
  source: /note=“Description of Artificial Sequence: Synthetic polynucleotide”
gatattcagctgacccagtctcctagcttcctgtccgcttctgtgggcgacagagtgaccatcacatgcaaggcctctcaggacgtgacctctgccgtggcttggtatcagcagaagcctggcaaggcccctaagctgctgatctactggacctccaccagacacaccggcgtgccctctagattttccggctctggctctggcaccgagtataccctgacaatctccagcctgcagcctgaggacttcgccacctactactgccagcagcactacaccacacctctgacctttggccagggcaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 288 - protein, 119 aa, Artificial Sequence
  source: /note=“Description of Artificial Sequence: Synthetic polypeptide”
QVQLQQSGPGLVQPSQSLSITCTVSGFSLTNYGVHWVRQSPGKGLEWLGVIWRGESTDYNAAFMSRLSVTKDDSKSQVFFKMNSLQADDTAIYYCARNGGSSGWYFDVWGTGTTVTVSS

SEQ ID NO: 289 - DNA, 357 bp, Artificial Sequence
  source: /note=“Description of Artificial Sequence: Synthetic polynucleotide”
caggtgcagctacagcagtcaggacctggcctagtgcagccctcacagagcctgtccataacctgcacagtctctggtttctcattaactaactatggtgtacactgggttcgccagtctccaggaaagggtctggagtggctgggagtgatatggagaggtgaaagcacagactacaatgcagctttcatgtccagactgagcgtcaccaaggacgactccaagagccaagttttctttaaaatgaacagtctgcaagctgatgacactgccatatactactgtgccagaaatggcggtagtagcgggtggtacttcgatgtctggggcacagggaccactgtcaccgtctcctca

SEQ ID NO: 290 - protein, 454 aa, Artificial Sequence
  source: /note=“Description of Artificial Sequence: Synthetic polypeptide”
QVQLQQSGPGLVQPSQSLSITCTVSGFSLTNYGVHWVRQSPGKGLEWLGVIWRGESTDYNAAFMSRLSVTKDDSKSQVFFKMNSLQADDTAIYYCARNGGSSGWYFDVWGTGTTVTVSSAKTTAPSVYPLAPVCGGTTGSSVTLGCLVKGYFPCPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALASPIEKTISKPRGPVRAPQVYVLPPPAEEMTKKEFSLTCMITGFLPCEIAVDWTSNGRTEQNYKNTATVLDSDGSYFMYSKLRVQKSTWERGSLFACSVVHEGLHNHLTTKTISRSLGK

SEQ ID NO: 291 - DNA, 1362 bp, Artificial Sequence
  source: /note=“Description of Artificial Sequence: Synthetic polynucleotide”
caggtgcagctacagcagtcaggacctggcctagtgcagccctcacagagcctgtccataacctgcacagtctctggtttctcattaactaactatggtgtacactgggttcgccagtctccaggaaagggtctggagtggctgggagtgatatggagaggtgaaagcacagactacaatgcagctttcatgtccagactgagcgtcaccaaggacgactccaagagccaagttttctttaaaatgaacagtctgcaagctgatgacactgccatatactactgtgccagaaatggcggtagtagcgggtggtacttcgatgtctggggcacagggaccactgtcaccgtctcctcagccaaaacaacagccccatcggtctatccactggcccctgtgtgtggaggtacaactggctcctcggtgactctaggatgcctggtcaagggttatttcccttgtccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggctgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcgcatcccccatcgagaaaaccatctcaaaacccagagggccagtaagagctccacaggtatatgtcttgcctccaccagcagaagagatgactaagaaagagttcagtctgacctgcatgatcacaggcttcttaccttgtgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 292 - protein, 107 aa, Artificial Sequence
  source: /note=“Description of Artificial Sequence: Synthetic polypeptide”
DIQMTQTHKFMSTSVGDRVSITCKASQDVTSAVAWYQQTPGQSPNLLIYWTSTRHTGVPDRFTGSGSGTDYTLTISSVQAEDLALYYCQQHYTTPLTFGAGTKLELK

SEQ ID NO: 293 - DNA, 321 bp, Artificial Sequence
  source: /note=“Description of Artificial Sequence: Synthetic polynucleotide”
gacattcagatgacccagactcacaaattcatgtccacatcagtaggagacagggtcagcatcacctgcaaggccagtcaggatgtgacttctgctgtagcctggtatcaacaaacaccaggacaatctcctaatctactgatttactggacatccacccggcacactggagtccctgatcgcttcacaggcagtggatctgggacagattatactctcaccatcagcagtgtgcaggctgaagacctggcactttattactgtcagcaacattataccactccgctcacgttcggtgctgggaccaagctggagctaaaa

SEQ ID NO: 294 - protein, 214 aa, Artificial Sequence
  source: /note=“Description of Artificial Sequence: Synthetic polypeptide”
DIQMTQTHKFMSTSVGDRVSITCKASQDVTSAVAWYQQTPGQSPNLLIYWTSTRHTGVPDRFTGSGSGTDYTLTISSVQAEDLALYYCQQHYTTPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 295 - DNA, 642 bp, Artificial Sequence
  source: /note=“Description of Artificial Sequence: Synthetic polynucleotide”
gacattcagatgacccagactcacaaattcatgtccacatcagtaggagacagggtcagcatcacctgcaaggccagtcaggatgtgacttctgctgtagcctggtatcaacaaacaccaggacaatctcctaatctactgatttactggacatccacccggcacactggagtccctgatcgcttcacaggcagtggatctgggacagattatactctcaccatcagcagtgtgcaggctgaagacctggcactttattactgtcagcaacattataccactccgctcacgttcggtgctgggaccaagctggagctaaaacgtgccgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 296 - protein, 11 aa, Artificial Sequence
  source: /note=“Description of Artificial Sequence: Synthetic peptide”
NAGSSGWYFDV

SEQ ID NO: 297 - protein, 13 aa, Artificial Sequence
  source: /note=“Description of Artificial Sequence: Synthetic peptide”
ARNAGSSGWYFDV

SEQ ID NO: 298 - protein, 119 aa, Artificial Sequence
  source: /note=“Description of Artificial Sequence: Synthetic polypeptide”
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNAGSSGWYFDVWGQGTTVTVSS

SEQ ID NO: 299 - DNA, 357 bp, Artificial Sequence
  source: /note=“Description of Artificial Sequence: Synthetic polynucleotide”
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacgctggctcctccggctggtactttgatgtgtggggccagggcaccaccgtgacagttagttct

SEQ ID NO: 300 - protein, 449 aa, Artificial Sequence
  source: /note=“Description of Artificial Sequence: Synthetic polypeptide”
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNAGSSGWYFDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 301 - DNA, 1347 bp, Artificial Sequence
  source: /note=“Description of Artificial Sequence: Synthetic polynucleotide”
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacgctggctcctccggctggtactttgatgtgtggggccagggcaccaccgtgacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 302 - DNA, 4328 bp, Homo sapiens
atcacagggtgggaaataaaagctgtggcccccaggagttctggacactgggggagagtggggtgacatgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtagcagaacttcacccccttttaagctacagttccttctctccatccttcgaccttcacaaaatctctgggactgttctttgtcagattcttcctcctttagaaggctgggtcccattctgtccttcttgtcatgcctccaatttcccctggtgtagagcttgtttttctggcccatccttggagctttatgagtgagctggtgtgggatgcctttgggggtggacttgtgttccaagaatccactctctcttccttttggagattaggatatttgggttgccatgtgtagctgctatgtcccctggggcgttatcttatacatgcaaacctaccatctgttcaacttccacctaccacctcctgcacccctttgatcggggacttactggttgcaagagctcattttgcaggctggaagcaccagggaattaattcccccagtcaaccaatggcacccagagagggcatggaggctccacgcaaccccttccacccccacatcttcctttgtcttatacatggcttccatttggctgtttctaagttgtattctttattttattattattattactatttttcgagatggagtttcactcttgtcgctcaggctggagtgccatggcgcgatcttggctcactgcaacctctgcctcccgggttcaagtgattctcctgcctcagcctcacgagtagctggaattacaggcaggcgccaccagacccggctaattttttgtatttttagtacagatggggtttctccgtgttggtcaggctggtcttgaactcccgacctcagatgatctgcccgcctcggcctcccaaaattgctgggattacaggtgtgagccaccgcgcctggcctattattttttgtaagaataaaacaggtttattgggatttgggactctgaacagttctgtctctactacctgatctcctcctaccacgactttgggatctagaggagctttggctccggctgtgacggctccggccgttctcactgcggctgcaccggcccccgctgcggtcactatttcttcctctgctaggtgaattgtgcctctcctggctctttgacatgtgctagtgagatttcttccttttcctttcggattccccatttcttttgtaggaatggtctggactagggttctccttccccgcagcctgtagtattcatcgtggtggcccaccctctctctccccttggagctcttgccaaaggaggagacaagcagaggtctctattggatttctcaacacctgaagaaagttgcagtgttttcctcttggacattgttgtatttcaaataaaccacaaatcatcattttccaccgagccactgggcagaattcacactgaagctgtcgtcctgcgtacataccatcgtccgttaaacagagaaagagctgcttggcattcttcttccgactggtactgaacatatatacttgcccctcaggtgaggttccaagttgcaactgaccttgaactgaatcactctccccacgttattttttaattactatttttttttaaagatggggtcttgctctgtcgccaggctggagtgcagtggcgcgatctaggctcactgcaacttccgcctcccgggttcaagcgattctcctgcctcagcctcccgagtagctgggactccactaaaagtacaaaaattagctgggcgtgcaccactgcgcccagctaattcttgtatttttggtagagacggggtttcaacatgttgaccaggatggtctcgatctcttgacctcgtgattcgcccgccgcgtcctcccaaagtgctgggattacaggcctgagccaccgcgcccagtctctccccacgttcttgaactcgggcagcacatcctcacagaaatctaggaactgttggtaggtttcttcctcgctgtactccaggcttgcttcggagtcatagtcatccctcctgcactgctcctttccaaacactgtaaacatgcttttaataagaagggtaggactggatgttgggaaatcatgtgaacatctatctccaaatctgcaagctcctgttttactgtagaagggacaattaactccatccttctccatgactctgaaatccaagggggggttccgggttttgccatgtggcgccattttccaactcattttcagcctgatccagcatcttctggacagcttccggtttttgtttcttctgtcgtttctgttcctcctcctctctctctttcctctgctgttcttcccattgttcctttaactttcgctcttgttcttgccgttttctagccacctcttccttttccttctttattctgaattcttcttgtgccttctgctctctcagcaaccactcctcatgtaatctttgcctctctcttccccatagcttttctagttgttgtttttcaataaaagtgtcctcctctttctgtgagagtcctgagtccctcagtggagcaagttcctgctggcgtttctttcgtttctccttcttcagggcggccctgtactttttgtggcttggtttctctggaaatgtcaccttttcgggcgcagccatcttgccggcaccgccccgcccctctagttgtatcctttataataaactggtaaacattgtaaccgcagattcagcccaatctggttcaactttgtgtaataaaatggcgagttgtttttcagttgtcgtggacccccaggttgcaagttacataccctgggcatgtccagatgaacgaagcgtgcaaatccacgtggaacctaagtgctcagaccgaggaacagggactgagttaagaagtggacaccacgtggcatgatccttgatccaatcagattgagccctggcgtgatccagtcagatcaagcctcctgaatcccctcattacaagatccaatcatatcatgcctcactaccctctgtatataaaatctgccccagcctccaacttggagagacagatttgggccagactcctgtgtccttgcttggctgccttgcaataaatttttctctctacaaaa

SEQ ID NO: 303 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 304 - DNA, 1976 bp, Homo sapiens
acccagcttcctgtttgtcttcctgagagacagtagatttagaaagtgaggatcagagggtggaaaataaaagctgtggtccccaggagtcctgaacatctggggacagcgggaaaacatgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatagttgtttccctgctagcctcagcctccattgtggtatagcagaacttcacccacttgtaagccagcgcttcttctctccatccttggaccttcacaaatgccctgagacggttctctgttcgatttttcatcccctatgaacctgggtcttattctgtccttctgatgcctccaagtttccctggtgtagagcttgtgttcttggcccatccttggagctttataagtgacctgagtgggatgcatttagggggcgggcttggtatgttgtatgaatccactctctgttccttttggagattagactatttggattcatgtgtagctgccctgtcccctggggctttatctcatccatgcaaactaccatctgctcaacttccagctacaccccgtgcacccttttgactggggacttgctggttgaaggagctcatcttgcaggctggaagcaccagggaattaattcccccagtcaaccaatggcatccagagagggcatggaggctccatacaacctcttccacccccacatctttctttgtcctatacatgtcttccatttggctgtttctgagttgtagcctttataataaagtggtaaatgttgtaactgcaaaaaaaaaaaaaaaaaaaaaaaaaaaaaaaaaaaaaaaaaa

SEQ ID NO: 305 - protein, 399 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 306 - DNA, 1212 bp, Homo sapiens
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcg

SEQ ID NO: 307 - protein, 343 aa, Artificial Sequence
  source: /note=“Description of Artificial Sequence: Synthetic polypeptide”
KVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 308 - DNA, 1029 bp, Artificial Sequence
  source: /note=“Description of Artificial Sequence: Synthetic polynucleotide”
aaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcg

SEQ ID NO: 309 - protein, 155 aa, Artificial Sequence
  source: /note=“Description of Artificial Sequence: Synthetic polypeptide”
ERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 310 - DNA, 465 bp, Artificial Sequence
  source: /note=“Description of Artificial Sequence: Synthetic polynucleotide”
gaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaaccctcctcctgcg

SEQ ID NO: 311 - protein, 406 aa, Artificial Sequence
  source: /note=“Description of Artificial Sequence: Synthetic polypeptide”
MSDSKEPRLQQLDLLEEEQLGGVGFRQTRGYKSLAGCLGHGPLVLQLLSFTLLAGLLVQVSKVPSSLSQGQSKQDAIYQNLTQLKVAVSELSEKSKQQEIYQELTRLKAAVGELPEKSKQQEIYEELTRLKAAVGELPEKSKLQEIYQELTRLKAAVGELPEKSKQQEIYQELSRLKAAVGDLPEKSKQQEIYQKLTQLKAAVDGLPDRSKQQEIYQELIQLKAAVDLEGWTDTGIWTTSSEPSPDRPPPTERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPPE

SEQ ID NO: 312 - DNA, 1224 bp, Artificial Sequence
  source: /note=“Description of Artificial Sequence: Synthetic polynucleotide”
atgtcggactcgaaggaaccaagactgcagcaactcgacctccttgaagaagaacagctcggcggagtgggattccggcagaccaggggttacaagagcctggccggttgcctgggtcacggccctttggtgcttcagctgctgtcgttcaccctgctggccggactgcttgtgcaagtctccaaagtcccgtcctcgctgagccaggggcagtccaagcaggacgcgatctaccaaaacctgacacagctcaaggtggccgtgtcagagctgtccgagaagtcgaagcagcaagagatctaccaagagttgacgcgactcaaagcagccgtgggcgaacttcccgagaagtcaaagcagcaggaaatctacgaggaattgacccgcctgaaggccgccgtgggagagctgccagaaaagtcgaagctgcaggagatataccaagaactcacccggctcaaggccgctgtgggagaactgccggagaagtccaaacaacaggaaatctaccaggaactgagcagactcaaggcagccgtcggcgatctccccgaaaagtctaaacagcaggagatctatcagaagctgactcagctgaaggcggccgtggacgggctgcccgatcggtccaagcaacaggaaatctaccaggagctgatccaactgaaggctgccgtggacctggaagggtggactgacaccgggatttggactacctcatcggaaccgagccctgatcgccctccgcctaccgagaggttgtgtcacccgtgcccatgggagtggacgttcttccaaggaaactgttactttatgagcaacagccagcggaattggcacgattccattaccgcgtgccaggaagtgggcgcccagctggtcgtgatcaagtccgcggaggagcagaacttcctgcagctccagagcagccggtccaaccgcttcacctggatgggcctctccgacctgaaccatgagggaacttggcagtgggtggacggttccccgctgctgccctcattcaagcagtactggaacaagggagaaccgaacaacgtcggagaggaagattgcgccgagttttccgggaacggatggaacgacgacaagtgcaatctggccaagttctggatttgcaagaagtccgctgcatcctgctcgggcgacgaggagcgcctgctgtcccccgcgcccaccacccctaaccctcccccggaatgatag

SEQ ID NO: 313 - protein, 336 aa, Artificial Sequence
  source: /note=“Description of Artificial Sequence: Synthetic polypeptide”
QPSKQDAIYQNLTQLKVAVSELSEKSKQQEIYQELTRLKAAVGELPEKSKQQEIYEELTRLKAAVGELPEKSKLQEIYQELTRLKAAVGELPEKSKQQEIYQELSRLKAAVGDLPEKSKQQEIYQKLTQLKAAVDGLPDRSKQQEIYQELIQLKAAVDLEGWTDTGIWTTSSEPSPDRPPPTERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPP

SEQ ID NO: 314 - DNA, 1005 bp, Artificial Sequence
  source: /note=“Description of Artificial Sequence: Synthetic polynucleotide”
tccaagcaggacgcgatctaccaaaacctgacacagctcaaggtggccgtgtcagagctgtccgagaagtcgaagcagcaagagatctaccaagagttgacgcgactcaaagcagccgtgggcgaacttcccgagaagtcaaagcagcaggaaatctacgaggaattgacccgcctgaaggccgccgtgggagagctgccagaaaagtcgaagctgcaggagatataccaagaactcacccggctcaaggccgctgtgggagaactgccggagaagtccaaacaacaggaaatctaccaggaactgagcagactcaaggcagccgtcggcgatctccccgaaaagtctaaacagcaggagatctatcagaagctgactcagctgaaggcggccgtggacgggctgcccgatcggtccaagcaacaggaaatctaccaggagctgatccaactgaaggctgccgtggacctggaagggtggactgacaccgggatttggactacctcatcggaaccgagccctgatcgccctccgcctaccgagaggttgtgtcacccgtgcccatgggagtggacgttcttccaaggaaactgttactttatgagcaacagccagcggaattggcacgattccattaccgcgtgccaggaagtgggcgcccagctggtcgtgatcaagtccgcggaggagcagaacttcctgcagctccagagcagccggtccaaccgcttcacctggatgggcctctccgacctgaaccatgagggaacttggcagtgggtggacggttccccgctgctgccctcattcaagcagtactggaacaagggagaaccgaacaacgtcggagaggaagattgcgccgagttttccgggaacggatggaacgacgacaagtgcaatctggccaagttctggatttgcaagaagtccgctgcatcctgctcgggcgacgaggagcgcctgctgtcccccgcgcccaccacccctaaccctcccccggaa

SEQ ID NO: 315 - protein, 157 aa, Artificial Sequence
  source: /note=“Description of Artificial Sequence: Synthetic polypeptide”
QPERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPPE

SEQ ID NO: 316 - DNA, 465 bp, Artificial Sequence
  source: /note=“Description of Artificial Sequence: Synthetic polynucleotide”
gagaggttgtgtcacccgtgcccatgggagtggacgttcttccaaggaaactgttactttatgagcaacagccagcggaattggcacgattccattaccgcgtgccaggaagtgggcgcccagctggtcgtgatcaagtccgcggaggagcagaacttcctgcagctccagagcagccggtccaaccgcttcacctggatgggcctctccgacctgaaccatgagggaacttggcagtgggtggacggttccccgctgctgccctcattcaagcagtactggaacaagggagaaccgaacaacgtcggagaggaagattgcgccgagttttccgggaacggatggaacgacgacaagtgcaatctggccaagttctggatttgcaagaagtccgctgcatcctgctcgggcgacgaggagcgcctgctgtcccccgcgcccaccacccctaaccctcccccggaa

SEQ ID NO: 317 - protein, 368 aa, Artificial Sequence
  source: /note=“Description of Artificial Sequence: Synthetic polypeptide”
KVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPAGSGGGLNDIFEAQKIEWHEHHHHHH

SEQ ID NO: 318 - protein, 198 aa, Artificial Sequence
  source: /note=“Description of Artificial Sequence: Synthetic polypeptide”
MQLLSCIALSLALVTNSTERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPAGSGGGLNDIFEAQKIEWHEHHHHHH

SEQ ID NO: 319 - protein, 384 aa, Artificial Sequence
  source: /note=“Description of Artificial Sequence: Synthetic polypeptide”
MKTFILLLWVLLLWVIFLLPGATAQPSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPADYKDDDDKHHHHHH

SEQ ID NO: 320 - protein, 11 aa, Artificial Sequence
  source: /note=“Description of Artificial Sequence: Synthetic peptide”
VVIKSAEEQNF

SEQ ID NO: 321 - protein, 19 aa, Artificial Sequence
  source: /note=“Description of Artificial Sequence: Synthetic peptide”
LQLQSSRSNRFTWMGLSDL

SEQ ID NO: 322 - protein, 15 aa, Artificial Sequence
  source: /note=“Description of Artificial Sequence: Synthetic peptide”
NQEGTWQWVDGSPLL

SEQ ID NO: 323 - protein, 18 aa, Artificial Sequence
  source: /note=“Description of Artificial Sequence: Synthetic peptide”
NQEGTWQWVDGSPLLPSF

SEQ ID NO: 324 - DNA, 2223 bp, Homo sapiens
tataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacccaaaaaaaaaaaaaaaaaa

SEQ ID NO: 325 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 326 - DNA, 1795 bp, Homo sapiens
gctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacccaaaaaaaaaaaaaaaaaa

SEQ ID NO: 327 - protein, 283 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRNLQMTAASRCPRRFSGTCGRRKRKRLLWAA

SEQ ID NO: 328 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 329 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 330 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 331 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcaccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 332 - protein, 5 aa, Artificial Sequence
  source: /note=“Description of Artificial Sequence: Synthetic peptide”
GGGGS

SEQ ID NO: 333 - protein, 5 aa, Artificial Sequence
  source: /note=“Description of Artificial Sequence: Synthetic peptide”
GGGGA

SEQ ID NO: 334 - RNA, 24 nt, Artificial Sequence
  source: /note=“Description of Artificial Sequence: Synthetic oligonucleotide”
ggacguacgcuucggcguacgucc

SEQ ID NO: 335 - RNA, 24 nt, Artificial Sequence
  source: /note=“Description of Artificial Sequence: Synthetic oligonucleotide”
ggacguacgcuucggcguacgucc

SEQ ID NO: 336 - RNA, 24 nt, Artificial Sequence
  source: /note=“Description of Artificial Sequence: Synthetic oligonucleotide”
ggacguacgcuucggcguacgucc

SEQ ID NO: 337 - RNA, 24 nt, Artificial Sequence
  source: /note=“Description of Artificial Sequence: Synthetic oligonucleotide”
ggacguacgcuucggcguacgucc

SEQ ID NO: 338 - RNA, 24 nt, Artificial Sequence
  source: /note=“Description of Artificial Sequence: Synthetic oligonucleotide”
ggacguacgcuucggcguacgucc

SEQ ID NO: 339 - RNA, 24 nt, Artificial Sequence
  source: /note=“Description of Artificial Sequence: Synthetic oligonucleotide”
ggacguacgcuucggcguacgucc

SEQ ID NO: 340-500 - skipped (161 entries)

SEQ ID NO: 501 - protein, 5 aa, Artificial Sequence
  source: /note=“Description of Artificial Sequence: Synthetic peptide”
TYWMH

SEQ ID NO: 502 - protein, 17 aa, Artificial Sequence
  source: /note=“Description of Artificial Sequence: Synthetic peptide”
NIYPGTGGSNFDEKFKN

SEQ ID NO: 503 - protein, 8 aa, Artificial Sequence
  source: /note=“Description of Artificial Sequence: Synthetic peptide”
WTTGTGAY

SEQ ID NO: 504 - protein, 7 aa, Artificial Sequence
  source: /note=“Description of Artificial Sequence: Synthetic peptide”
GYTFTTY

SEQ ID NO: 505 - protein, 6 aa, Artificial Sequence
  source: /note=“Description of Artificial Sequence: Synthetic peptide”
YPGTGG

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  source: /note=“Description of Artificial Sequence: Synthetic peptide”
KSSQSLLDSGNQKNFLT

SEQ ID NO: 511 - protein, 7 aa, Artificial Sequence
  source: /note=“Description of Artificial Sequence: Synthetic peptide”
WASTRES

SEQ ID NO: 512 - protein, 9 aa, Artificial Sequence
  source: /note=“Description of Artificial Sequence: Synthetic peptide”
QNDYSYPYT

SEQ ID NO: 513 - protein, 13 aa, Artificial Sequence
  source: /note=“Description of Artificial Sequence: Synthetic peptide”
SQSLLDSGNQKNF

SEQ ID NO: 514 - protein, 3 aa, Artificial Sequence
  source: /note=“Description of Artificial Sequence: Synthetic peptide”
WAS

SEQ ID NO: 515 - protein, 6 aa, Artificial Sequence
  source: /note=“Description of Artificial Sequence: Synthetic peptide”
DYSYPY

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  source: /note=“Description of Artificial Sequence: Synthetic oligonucleotide”
acctactggatgcac

SEQ ID NO: 525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 526 - DNA, 24 bp, Artificial Sequence
  source: /note=“Description of Artificial Sequence: Synthetic oligonucleotide”
tggactaccggcacaggcgcctac

SEQ ID NO: 527 - DNA, 21 bp, Artificial Sequence
  source: /note=“Description of Artificial Sequence: Synthetic oligonucleotide”
ggctacaccttcactacctac

SEQ ID NO: 528 - DNA, 18 bp, Artificial Sequence
  source: /note=“Description of Artificial Sequence: Synthetic oligonucleotide”
taccccggcaccggcggc

SEQ ID NO: 529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 530 - DNA, 21 bp, Artificial Sequence
  source: /note=“Description of Artificial Sequence: Synthetic oligonucleotide”
tgggcctctactagagaatca

SEQ ID NO: 531 - DNA, 27 bp, Artificial Sequence
  source: /note=“Description of Artificial Sequence: Synthetic oligonucleotide”
cagaacgactatagctacccctacacc

SEQ ID NO: 532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 533 - DNA, 9 bp, Artificial Sequence
  source: /note=“Description of Artificial Sequence: Synthetic oligonucleotide”
tgggcctct

SEQ ID NO: 534 - DNA, 18 bp, Artificial Sequence
  source: /note=“Description of Artificial Sequence: Synthetic oligonucleotide”
gactatagctacccctac

SEQ ID NO: 535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541-600 - skipped (60 entries)

SEQ ID NO: 601 - protein, 5 aa, Artificial Sequence
  source: /note=“Description of Artificial Sequence: Synthetic peptide”
SYWMY

SEQ ID NO: 602 - protein, 17 aa, Artificial Sequence
  source: /note=“Description of Artificial Sequence: Synthetic peptide”
RIDPNSGSTKYNEKFKN

SEQ ID NO: 603 - protein, 11 aa, Artificial Sequence
  source: /note=“Description of Artificial Sequence: Synthetic peptide”
DYRKGLYAMDY

SEQ ID NO: 604 - protein, 7 aa, Artificial Sequence
  source: /note=“Description of Artificial Sequence: Synthetic peptide”
GYTFTSY

SEQ ID NO: 605 - protein, 6 aa, Artificial Sequence
  source: /note=“Description of Artificial Sequence: Synthetic peptide”
DPNSGS

SEQ ID NO: 606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, Artificial Sequence
  source: /note=“Description of Artificial Sequence: Synthetic peptide”
KASQDVGTAVA

SEQ ID NO: 610 - protein, 7 aa, Artificial Sequence
  source: /note=“Description of Artificial Sequence: Synthetic peptide”
WASTRHT

SEQ ID NO: 611 - protein, 9 aa, Artificial Sequence
  source: /note=“Description of Artificial Sequence: Synthetic peptide”
QQYNSYPLT

SEQ ID NO: 612 - protein, 7 aa, Artificial Sequence
  source: /note=“Description of Artificial Sequence: Synthetic peptide”
SQDVGTA

SEQ ID NO: 613 - protein, 3 aa, Artificial Sequence
  source: /note=“Description of Artificial Sequence: Synthetic peptide”
WAS

SEQ ID NO: 614 - protein, 6 aa, Artificial Sequence
  source: /note=“Description of Artificial Sequence: Synthetic peptide”
YNSYPL

SEQ ID NO: 615 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, Artificial Sequence
  source: /note=“Description of Artificial Sequence: Synthetic oligonucleotide”
agctactggatgtac

SEQ ID NO: 629 - DNA, 51 bp, Artificial Sequence
  source: /note=“Description of Artificial Sequence: Synthetic oligonucleotide”
agaatcgaccctaatagcggctctactaagtataacgagaagtttaagaat

SEQ ID NO: 630 - DNA, 33 bp, Artificial Sequence
  source: /note=“Description of Artificial Sequence: Synthetic oligonucleotide”
gactatagaaagggcctgtacgctatggactac

SEQ ID NO: 631 - DNA, 21 bp, Artificial Sequence
  source: /note=“Description of Artificial Sequence: Synthetic oligonucleotide”
ggctacaccttcactagctac

SEQ ID NO: 632 - DNA, 18 bp, Artificial Sequence
  source: /note=“Description of Artificial Sequence: Synthetic oligonucleotide”
gaccctaatagcggctct

SEQ ID NO: 633 - DNA, 33 bp, Artificial Sequence
  source: /note=“Description of Artificial Sequence: Synthetic oligonucleotide”
aaagcctctcaggacgtgggcaccgccgtggcc

SEQ ID NO: 634 - DNA, 21 bp, Artificial Sequence
  source: /note=“Description of Artificial Sequence: Synthetic oligonucleotide”
tgggcctctactagacacacc

SEQ ID NO: 635 - DNA, 27 bp, Artificial Sequence
  source: /note=“Description of Artificial Sequence: Synthetic oligonucleotide”
cagcagtataatagctaccccctgacc

SEQ ID NO: 636 - DNA, 21 bp, Artificial Sequence
  source: /note=“Description of Artificial Sequence: Synthetic oligonucleotide”
tctcaggacgtgggcaccgcc

SEQ ID NO: 637 - DNA, 9 bp, Artificial Sequence
  source: /note=“Description of Artificial Sequence: Synthetic oligonucleotide”
tgggcctct

SEQ ID NO: 638 - DNA, 18 bp, Artificial Sequence
  source: /note=“Description of Artificial Sequence: Synthetic oligonucleotide”
tataatagctaccccctg

SEQ ID NO: 639 - protein, 448 aa, Artificial Sequence
  source: /note=“Description of Artificial Sequence: Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, Artificial Sequence
  source: /note=“Description of Artificial Sequence: Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647-700 - skipped (54 entries)

SEQ ID NO: 701 - protein, 5 aa, Artificial Sequence
  source: /note=“Description of Artificial Sequence: Synthetic peptide”
NYGMN

SEQ ID NO: 702 - protein, 17 aa, Artificial Sequence
  source: /note=“Description of Artificial Sequence: Synthetic peptide”
WINTDTGEPTYADDFKG

SEQ ID NO: 703 - protein, 16 aa, Artificial Sequence
  source: /note=“Description of Artificial Sequence: Synthetic peptide”
NPPYYYGTNNAEAMDY

SEQ ID NO: 704 - protein, 7 aa, Artificial Sequence
  source: /note=“Description of Artificial Sequence: Synthetic peptide”
GFTLTNY

SEQ ID NO: 705 - protein, 6 aa, Artificial Sequence
  source: /note=“Description of Artificial Sequence: Synthetic peptide”
NTDTGE

SEQ ID NO: 706 - protein, 125 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, Artificial Sequence
  source: /note=“Description of Artificial Sequence: Synthetic peptide”
SSSQDISNYLN

SEQ ID NO: 711 - protein, 7 aa, Artificial Sequence
  source: /note=“Description of Artificial Sequence: Synthetic peptide”
YTSTLHL

SEQ ID NO: 712 - protein, 9 aa, Artificial Sequence
  source: /note=“Description of Artificial Sequence: Synthetic peptide”
QQYYNLPWT

SEQ ID NO: 713 - protein, 7 aa, Artificial Sequence
  source: /note=“Description of Artificial Sequence: Synthetic peptide”
SQDISNY

SEQ ID NO: 714 - protein, 3 aa, Artificial Sequence
  source: /note=“Description of Artificial Sequence: Synthetic peptide”
YTS

SEQ ID NO: 715 - protein, 6 aa, Artificial Sequence
  source: /note=“Description of Artificial Sequence: Synthetic peptide”
YYNLPW

SEQ ID NO: 716 - DNA, 1353 bp, Artificial Sequence
  source: /note=“Description of Artificial Sequence: Synthetic polynucleotide”
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, Artificial Sequence
  source: /note=“Description of Artificial Sequence: Synthetic polynucleotide”
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, Artificial Sequence
  source: /note=“Description of Artificial Sequence: Synthetic polynucleotide”
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, Artificial Sequence
  source: /note=“Description of Artificial Sequence: Synthetic polynucleotide”
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, Artificial Sequence
  source: /note=“Description of Artificial Sequence: Synthetic polynucleotide”
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, Artificial Sequence
  source: /note=“Description of Artificial Sequence: Synthetic oligonucleotide”
aattacgggatgaac

SEQ ID NO: 737 - DNA, 15 bp, Artificial Sequence
  source: /note=“Description of Artificial Sequence: Synthetic oligonucleotide”
aactacggcatgaac

SEQ ID NO: 738 - DNA, 51 bp, Artificial Sequence
  source: /note=“Description of Artificial Sequence: Synthetic oligonucleotide”
tggattaacaccgacaccggggagcctacctacgcggacgatttcaaggga

SEQ ID NO: 739 - DNA, 51 bp, Artificial Sequence
  source: /note=“Description of Artificial Sequence: Synthetic oligonucleotide”
tggatcaacaccgacaccggcgagcctacctacgccgacgacttcaagggc

SEQ ID NO: 740 - DNA, 48 bp, Artificial Sequence
  source: /note=“Description of Artificial Sequence: Synthetic oligonucleotide”
aacccgccctactactacggaaccaacaacgccgaagccatggactac

SEQ ID NO: 741 - DNA, 48 bp, Artificial Sequence
  source: /note=“Description of Artificial Sequence: Synthetic oligonucleotide”
aaccccccttactactacggcaccaacaacgccgaggccatggactat

SEQ ID NO: 742 - DNA, 21 bp, Artificial Sequence
  source: /note=“Description of Artificial Sequence: Synthetic oligonucleotide”
ggattcaccctcaccaattac

SEQ ID NO: 743 - DNA, 21 bp, Artificial Sequence
  source: /note=“Description of Artificial Sequence: Synthetic oligonucleotide”
ggcttcaccctgaccaactac

SEQ ID NO: 744 - DNA, 18 bp, Artificial Sequence
  source: /note=“Description of Artificial Sequence: Synthetic oligonucleotide”
aacaccgacaccggggag

SEQ ID NO: 745 - DNA, 18 bp, Artificial Sequence
  source: /note=“Description of Artificial Sequence: Synthetic oligonucleotide”
aacaccgacaccggcgag

SEQ ID NO: 746 - DNA, 33 bp, Artificial Sequence
  source: /note=“Description of Artificial Sequence: Synthetic oligonucleotide”
agctctagtcaggatatctctaactacctgaac

SEQ ID NO: 747 - DNA, 33 bp, Artificial Sequence
  source: /note=“Description of Artificial Sequence: Synthetic oligonucleotide”
tcctccagccaggacatctccaactacctgaac

SEQ ID NO: 748 - DNA, 21 bp, Artificial Sequence
  source: /note=“Description of Artificial Sequence: Synthetic oligonucleotide”
tacactagcaccctgcacctg

SEQ ID NO: 749 - DNA, 21 bp, Artificial Sequence
  source: /note=“Description of Artificial Sequence: Synthetic oligonucleotide”
tacacctccaccctgcacctg

SEQ ID NO: 750 - DNA, 27 bp, Artificial Sequence
  source: /note=“Description of Artificial Sequence: Synthetic oligonucleotide”
cagcagtactataacctgccctggacc

SEQ ID NO: 751 - DNA, 27 bp, Artificial Sequence
  source: /note=“Description of Artificial Sequence: Synthetic oligonucleotide”
cagcagtactacaacctgccctggacc

SEQ ID NO: 752 - DNA, 21 bp, Artificial Sequence
  source: /note=“Description of Artificial Sequence: Synthetic oligonucleotide”
agtcaggatatctctaactac

SEQ ID NO: 753 - DNA, 21 bp, Artificial Sequence
  source: /note=“Description of Artificial Sequence: Synthetic oligonucleotide”
agccaggacatctccaactac

SEQ ID NO: 754 - DNA, 9 bp, Artificial Sequence
  source: /note=“Description of Artificial Sequence: Synthetic oligonucleotide”
tacactagc

SEQ ID NO: 755 - DNA, 9 bp, Artificial Sequence
  source: /note=“Description of Artificial Sequence: Synthetic oligonucleotide”
tacacctcc

SEQ ID NO: 756 - DNA, 18 bp, Artificial Sequence
  source: /note=“Description of Artificial Sequence: Synthetic oligonucleotide”
tactataacctgccctgg

SEQ ID NO: 757 - DNA, 18 bp, Artificial Sequence
  source: /note=“Description of Artificial Sequence: Synthetic oligonucleotide”
tactacaacctgccctgg

SEQ ID NO: 758 - DNA, 15 bp, Artificial Sequence
  source: /note=“Description of Artificial Sequence: Synthetic oligonucleotide”
aactacgggatgaac

SEQ ID NO: 759 - DNA, 51 bp, Artificial Sequence
  source: /note=“Description of Artificial Sequence: Synthetic oligonucleotide”
tggattaacaccgacaccggcgagcctacctacgccgacgactttaagggc

SEQ ID NO: 760 - DNA, 48 bp, Artificial Sequence
  source: /note=“Description of Artificial Sequence: Synthetic oligonucleotide”
aaccccccctactactacggcactaacaacgccgaggctatggactac

SEQ ID NO: 761 - DNA, 21 bp, Artificial Sequence
  source: /note=“Description of Artificial Sequence: Synthetic oligonucleotide”
ggcttcaccctgactaactac

SEQ ID NO: 762 - protein, 447 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  source: /note=“Description of Artificial Sequence: Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  source: /note=“Description of Artificial Sequence: Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766-800 - skipped (35 entries)

SEQ ID NO: 801 - protein, 5 aa, Artificial Sequence
  source: /note=“Description of Artificial Sequence: Synthetic peptide”
SYNMH

SEQ ID NO: 802 - protein, 17 aa, Artificial Sequence
  source: /note=“Description of Artificial Sequence: Synthetic peptide”
DIYPGNGDTSYNQKFKG

SEQ ID NO: 803 - protein, 9 aa, Artificial Sequence
  source: /note=“Description of Artificial Sequence: Synthetic peptide”
VGGAFPMDY

SEQ ID NO: 804 - protein, 7 aa, Artificial Sequence
  source: /note=“Description of Artificial Sequence: Synthetic peptide”
GYTFTSY

SEQ ID NO: 805 - protein, 6 aa, Artificial Sequence
  source: /note=“Description of Artificial Sequence: Synthetic peptide”
YPGNGD

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  source: /note=“Description of Artificial Sequence: Synthetic peptide”
RASESVEYYGTSLMQ

SEQ ID NO: 811 - protein, 7 aa, Artificial Sequence
  source: /note=“Description of Artificial Sequence: Synthetic peptide”
AASNVES

SEQ ID NO: 812 - protein, 9 aa, Artificial Sequence
  source: /note=“Description of Artificial Sequence: Synthetic peptide”
QQSRKDPST

SEQ ID NO: 813 - protein, 11 aa, Artificial Sequence
  source: /note=“Description of Artificial Sequence: Synthetic peptide”
SESVEYYGTSL

SEQ ID NO: 814 - protein, 3 aa, Artificial Sequence
  source: /note=“Description of Artificial Sequence: Synthetic peptide”
AAS

SEQ ID NO: 815 - protein, 6 aa, Artificial Sequence
  source: /note=“Description of Artificial Sequence: Synthetic peptide”
SRKDPS

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  source: /note=“Description of Artificial Sequence: Synthetic peptide”
DIYPGQGDTSYNQKFKG

SEQ ID NO: 821 - protein, 6 aa, Artificial Sequence
  source: /note=“Description of Artificial Sequence: Synthetic peptide”
YPGQGD

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, Artificial Sequence
  source: /note=“Description of Artificial Sequence: Synthetic polynucleotide”
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, Artificial Sequence
  source: /note=“Description of Artificial Sequence: Synthetic polynucleotide”
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, Artificial Sequence
  source: /note=“Description of Artificial Sequence: Synthetic peptide”
SYGVD

SEQ ID NO: 910 - protein, 7 aa, Artificial Sequence
  source: /note=“Description of Artificial Sequence: Synthetic peptide”
GFSLSSY

SEQ ID NO: 911 - protein, 16 aa, Artificial Sequence
  source: /note=“Description of Artificial Sequence: Synthetic peptide”
VIWGGGGTYYASSLMG

SEQ ID NO: 912 - protein, 5 aa, Artificial Sequence
  source: /note=“Description of Artificial Sequence: Synthetic peptide”
WGGGG

SEQ ID NO: 913 - protein, 13 aa, Artificial Sequence
  source: /note=“Description of Artificial Sequence: Synthetic peptide”
HAYGHDGGFAMDY

SEQ ID NO: 914 - protein, 11 aa, Artificial Sequence
  source: /note=“Description of Artificial Sequence: Synthetic peptide”
RASESVSSNVA

SEQ ID NO: 915 - protein, 7 aa, Artificial Sequence
  source: /note=“Description of Artificial Sequence: Synthetic peptide”
SESVSSN

SEQ ID NO: 916 - protein, 7 aa, Artificial Sequence
  source: /note=“Description of Artificial Sequence: Synthetic peptide”
GASNRAT

SEQ ID NO: 917 - protein, 3 aa, Artificial Sequence
  source: /note=“Description of Artificial Sequence: Synthetic peptide”
GAS

SEQ ID NO: 918 - protein, 9 aa, Artificial Sequence
  source: /note=“Description of Artificial Sequence: Synthetic peptide”
GQSYSYPFT

SEQ ID NO: 919 - protein, 6 aa, Artificial Sequence
  source: /note=“Description of Artificial Sequence: Synthetic peptide”
SYSYPF

SEQ ID NO: 920 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 923 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 924 - protein, 114 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 925 - protein, 297 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 926 - protein, 114 aa, Artificial Sequence; identical to SEQ ID NO: 922
  source: /note=“Description of Artificial Sequence: Synthetic polypeptide”
  VARIANT (93)..(93): /replace=“Lys”
  SITE (1)..(114): /note=“Variant residues given in the sequence have no preference with respect to those in the annotations for variant positions”

SEQ ID NO: 927 - protein, 77 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIRDPALVHQRPAPP

SEQ ID NO: 928 - protein, 6 aa, Artificial Sequence
  source: /note=“Description of Artificial Sequence: Synthetic 6xHis tag”
HHHHHH

SEQ ID NO: 929 - protein, 60 aa, Artificial Sequence
  source: /note=“Description of Artificial Sequence: Synthetic polypeptide”
  SITE (1)..(5): /note=“This region may encompass 1-5 residues”
  SITE (7)..(11): /note=“This region may encompass 1-5 residues”
  SITE (13)..(17): /note=“This region may encompass 1-5 residues”
  SITE (19)..(23): /note=“This region may encompass 1-5 residues”
  SITE (25)..(29): /note=“This region may encompass 1-5 residues”
  SITE (31)..(35): /note=“This region may encompass 1-5 residues”
  SITE (37)..(41): /note=“This region may encompass 1-5 residues”
  SITE (43)..(47): /note=“This region may encompass 1-5 residues”
  SITE (49)..(53): /note=“This region may encompass 1-5 residues”
  SITE (55)..(59): /note=“This region may encompass 1-5 residues”
  SITE (1)..(60): /note=“This sequence may encompass 0-10 ′(Gly) n-Ser′ repeating units, wherein n is 1-5”
GGGGGSGGGGGSGGGGGSGGGGGSGGGGGSGGGGGSGGGGGSGGGGGSGGGGGSGGGGGS

SEQ ID NO: 930 - protein, 60 aa, Artificial Sequence
  source: /note=“Description of Artificial Sequence: Synthetic polypeptide”
  SITE (1)..(5): /note=“This region may encompass 1-5 residues”
  SITE (7)..(11): /note=“This region may encompass 1-5 residues”
  SITE (13)..(17): /note=“This region may encompass 1-5 residues”
  SITE (19)..(23): /note=“This region may encompass 1-5 residues”
  SITE (25)..(29): /note=“This region may encompass 1-5 residues”
  SITE (31)..(35): /note=“This region may encompass 1-5 residues”
  SITE (37)..(41): /note=“This region may encompass 1-5 residues”
  SITE (43)..(47): /note=“This region may encompass 1-5 residues”
  SITE (49)..(53): /note=“This region may encompass 1-5 residues”
  SITE (55)..(59): /note=“This region may encompass 1-5 residues”
  SITE (1)..(60): /note=“This sequence may encompass 0-10 ′ (Gly)n-Ala′ repeating units, wherein n is 1-5”
GGGGGAGGGGGAGGGGGAGGGGGAGGGGGAGGGGGAGGGGGAGGGGGAGGGGGAGGGGGA

SEQ ID NO: 931 - protein, 50 aa, Artificial Sequence
  source: /note=“Description of Artificial Sequence: Synthetic polypeptide”
  SITE (1)..(50): /note=“This sequence may encompass 0-10 ′Gly-Gly-Gly-Gly-Ser′ repeating units”
GGGGSGGGGSGGGGSGGGGSGGGGSGGGGSGGGGSGGGGSGGGGSGGGGS

SEQ ID NO: 932 - protein, 50 aa, Artificial Sequence
  source: /note=“Description of Artificial Sequence: Synthetic polypeptide”
  SITE (1)..(50): /note=“This sequence may encompass 0-10 ′Gly-Gly-Gly-Gly-Ala′ repeating units”
GGGGAGGGGAGGGGAGGGGAGGGGAGGGGAGGGGAGGGGAGGGGAGGGGA

SEQ ID NO: 933 - protein, 15 aa, Artificial Sequence
  source: /note=“Description of Artificial Sequence: Synthetic peptide”
GGGGSGGGGSGGGGS

SEQ ID NO: 934 - protein, 20 aa, Artificial Sequence
  source: /note=“Description of Artificial Sequence: Synthetic peptide”
GGGGSGGGGSGGGGSGGGGS

SEQ ID NO: 935 - protein, 5 aa, Artificial Sequence
  source: /note=“Description of Artificial Sequence: Synthetic peptide”
GGGGA

SEQ ID NO: 936 - RNA, 24 nt, Artificial Sequence
  source: /note=“Description of Artificial Sequence: Synthetic oligonucleotide”
ggacguacgcuucggcguacgucc

SEQ ID NO: 937 - RNA, 24 nt, Artificial Sequence
  source: /note=“Description of Artificial Sequence: Synthetic oligonucleotide”
  modified_base (12)..(12): a, c, u, g, unknown or other
ggacguacgcuncggcguacgucc

SEQ ID NO: 938 - protein, 4 aa, Artificial Sequence
  source: /note=“Description of Artificial Sequence: Synthetic peptide”
RGDS

SEQ ID NO: 939 - protein, 22 aa, Artificial Sequence
  source: /note=“Description of Artificial Sequence: Synthetic peptide”
YGRKKRRQRRRLYRSPAMPENL

(19) United States
(12) Patent Application Publication
(10) Pub. No.: US 2023/0053449 A1
(43) Pub. Date: Feb. 23, 2023

(54) DC-SIGN ANTIBODY DRUG CONJUGATES

(71) Applicant: NOVARTIS AG, Basel (CH)

(72) Inventors: Lisa BARNETT, Poway, CA (US); Steven BENDER, Oceanside, CA (US); Alex Cortez, San Diego, CA (US); Sarah COX, San Diego, CA (US); Jonathan DEANE, San Diego, CA (US); Scott Martin GLASER, San Diego, CA (US); Ben WEN, Encinitas, CA (US); Tom Yao-Hsiang WU, San Diego, CA (US)

(21) Appl. No.: 17/289,058

(22) PCT Filed: Oct. 30, 2019
(86) PCT No.: PCT/IB2019/059312

§ 371 Date: Apr. 27, 2021

Related U.S. Application Data
(60) Provisional application No. 62/753,655, filed on Oct. 31, 2018.

(51) Int. Cl. A61K 47/68; C07K 16/28; A61P 35/00

(52) U.S. Cl. CPC A61K 47/6803; C07K 16/2851; A61K 47/6849; A61K 2039/505; C07K 2319/74; C07K 2317/24; A61P 35/00

(57) ABSTRACT

Provided herein are antibodies to DC-SIGN, conjugates of DC-SiGN antibodies, and DC-SiGN antibody fusion proteins and the use of such antibodies, conjugates, and fusion proteins for the treatment of diseases such as cancer.

### CROSS-REFERENCE TO RELATED APPLICATIONS

This application claims the benefit of U.S. Provisional Application No. 62/753,655 filed Oct. 31, 2018, the content of which are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 16, 2019, is named PAT058305-WO-PCT_SL.txt and is 549,813 bytes in size.

### FIELD OF THE INVENTION

The invention provides anti-DC-SIGN antibodies, antibody conjugates comprising an anti-DC-SIGN antibody, and DC-SIGN antibody fusion proteins and their uses for the treatment of diseases such as cancer.

### BACKGROUND OF THE INVENTION

Dendritic Cell-Specific Intercellular adhesion molecule-3-Grabbing Non-integrin (DC-SIGN) is a C-type lectin receptor present on the surface of both macrophages and dendritic cells (Soilleux E J, et al. (2002) J Luekoc Biol. 71(3):445-57). DC-SIGN recognizes and binds to mannose containing carbohydrates, a class of pathogen-associated molecular patterns (PAMPs) commonly found on viruses, bacteria and fungi. This binding interaction activates phagocytic uptake and internalization of pathogens (McGreal E, et al. (2005) Curr Opin Immunol. 17 (1): 18-24, Engering A, et al. (2002) J Immunol. 168(5):2118-26). Additionally, on myeloid and pre-plasmacytoid dendritic cells, DC-SIGN mediates dendritic cell rolling interactions with blood endothelium and activation of CD4+ T cells (Geijtenbeek T, et al. (2000) Cell 100(5):575-85.

Besides functioning as an adhesion and internalization molecule, recent studies have also shown that DC-SIGN can initiate innate immunity by modulating toll-like receptors (den Dunnen J, et al. (2009) Cancer Immunol. Immunother. 58 (7): 1149-57), though the detailed mechanism is not yet known. Innate immunity is a rapid nonspecific immune response that fights against environmental insults including, but not limited to, pathogens such as bacteria or viruses. Adaptive immunity is a slower but more specific immune response, which confers long-lasting or protective immunity to the host and involves differentiation and activation of naive T lymphocytes into CD4+T helper cells and/or CD8+ cytotoxic T cells, promoting cellular and humoral immunity. Antigen presentation cells of the innate immune system, such as dendritic cells or macrophages, thus serve as a critical link between the innate and adaptive immune systems by phagocytosing and processing the foreign antigens and presenting them on the cell surface to T cells, thereby activating T cell responses. In cancer biology, DC-SIGN, together with other C-type lectins, is involved in recognition of tumors by dendritic cells and considered to play a critical role in tumor-associated immune responses (van Gisbergen K P et al. (2005) Cancer Res 65(13):5935-44). Additionally, dendritic cells in the tumor microenvironment are often negatively influenced by the surrounding tumor cells and develop a suppressive phenotype (Janco J M et al. (2015) J Immunol. 194(7): 2985-2991). Novel therapies that are able to induce dendritic cell activation represent an important class of potential cancer treatments. Consequently, dendritic cells, and particularly DC-SIGN, are important targets for developing novel cancer immunotherapy treatments.

Despite the development of a multitude of effective biologic, small molecule, and more recently cell-based therapeutics for treating cancer significant clinical challenges, such as tumor heterogeneity, acquired resistance, and subpopulation patient responsiveness remain. There remains an urgent need for new immunotherapies for the treatment of diseases, in particular cancer.

### SUMMARY OF THE INVENTION

The invention is based on the finding that targeting dendritic cells and macrophages, by way of the C-type lectin receptor DC-SIGN, with an antibody conjugated to a TLR agonist agent or RIG-1 agonist, induces potent dendritic cell and macrophage activation. The unique combination of a DC-SIGN targeting agent and a TLR agonist or RIG-1 agonist, engineered as a single therapeutic agent, may provide greater clinical benefit as compared to combinations of single agents alone.

The invention provides an antibody or antigen binding fragment thereof that binds to human DC-SIGN protein, wherein the antibody or antigen binding fragment thereof has a higher affinity to human DC-SIGN than human L-SIGN. In some embodiments disclosed herein, the antibody or antigen binding fragment thereof has an affinity to human DC-SIGN that is 10× higher than human L-SIGN. In some embodiments disclosed herein, the antibody or antigen binding fragment thereof has an affinity to human DC-SIGN that is 100× higher than human L-SIGN. In some embodiments, the Ab specifically binds to human DC-SIGN. In some embodiments, the Ab does not bind to human L-SIGN. In some embodiments, the antibody or antigen binding fragment thereof has a reduced level of, or no significant level of antibody-dependent cell-mediated cytotoxicity (ADCC) activity. In some embodiments, the antibody or antigen binding fragment thereof comprises a silenced Fc region. In some embodiments, the antibody or antigen binding fragment thereof comprises a mutation in the Fc region selected from: D265A; P329A; P329G; N297A; D265A and P329A; D265A and N297A; L234 and L235A; P329A, L234A and L235A; and P329G, L234A and L235A. In some embodiments, the antibody or antigen binding fragment thereof has no significant cell killing activity. In some embodiments, the antibody or antigen binding fragment thereof binds to an epitope having the amino acid sequence of SEQ ID NOs: 320-323. In some embodiments, the Ab is human or humanized. In other embodiments, the Ab is a monoclonal antibody.

In some embodiments, the antibody or antigen binding fragment thereof comprises one or more cysteine substitutions. In some embodiments, the antibody or antigen binding fragment thereof comprises one or more cysteine substitutions selected from S152C, S375C, or both S152C and S375C of the heavy chain of the antibody or antigen binding fragment thereof, wherein the position is numbered according to the EU system. In some embodiments, the antibody or antigen binding fragment thereof comprises a cysteine substitution of S152C of the heavy chain of the antibody or antigen binding fragment thereof, wherein the position is numbered according to the EU system. In some embodiments, the antibody or antigen binding fragment thereof comprises a cysteine substitution of S375C of the heavy chain of the antibody or antigen binding fragment thereof, wherein the position is numbered according to the EU system.

The invention provides an antibody or antigen binding fragment thereof that binds DC-SIGN comprising:
a. a heavy chain variable region that comprises an HCDR1 (Heavy Chain Complementarity Determining Region 1) of SEQ ID NO:1, an HCDR2 (Heavy Chain Complementarity Determining Region 2) of SEQ ID NO:2, and an HCDR3 (Heavy Chain Complementarity Determining Region 3) of SEQ ID NO:3; and a light chain variable region that comprises an LCDR1 (Light Chain Complementarity Determining Region 1) of SEQ ID NO:14, an LCDR2 (Light Chain Complementarity Determining Region 2) of SEQ ID NO:15, and an LCDR3 (Light Chain Complementarity Determining Region 3) of SEQ ID NO:16;
b. a heavy chain variable region that comprises an HCDR1 of SEQ ID NO:25, an HCDR2 of SEQ ID NO:26, and an HCDR3 of SEQ ID NO:27; and a light chain variable region that comprises an LCDR1 of SEQ ID NO:38, an LCDR2 of SEQ ID NO:39, and an LCDR3 of SEQ ID NO:40;
c. a heavy chain variable region that comprises an HCDR1 of SEQ ID NO:49, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:60;
d. a heavy chain variable region that comprises an HCDR1 of SEQ ID NO:74, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:82;
e. a heavy chain variable region that comprises an HCDR1 of SEQ ID NO:88, an HCDR2 of SEQ ID NO:26, and an HCDR3 of SEQ ID NO:50; and a light chain variable region that comprises an LCDR1 of SEQ ID NO:94, an LCDR2 of SEQ ID NO:95, and an LCDR3 of SEQ ID NO:82;
f. a heavy chain variable region that comprises an HCDR1 of SEQ ID NO: 111, an HCDR2 of SEQ ID NO:26, and an HCDR3 of SEQ ID NO:27; and a light chain variable region that comprises an LCDR1 of SEQ ID NO:38, an LCDR2 of SEQ ID NO:39, and an LCDR3 of SEQ ID NO:118;
g. a heavy chain variable region that comprises an HCDR1 of SEQ ID NO:49, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:124;
h. a heavy chain variable region that comprises an HCDR1 of SEQ ID NO:74, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:124;
i. a heavy chain variable region that comprises an HCDR1 of SEQ ID NO:88, an HCDR2 of SEQ ID NO:26, and an HCDR3 of SEQ ID NO:50; and a light chain variable region that comprises an LCDR1 of SEQ ID NO:94, an LCDR2 of SEQ ID NO:95, and an LCDR3 of SEQ ID NO:124;
j. a heavy chain variable region that comprises an HCDR1 of SEQ ID NO:138, an HCDR2 of SEQ ID NO:139, and an HCDR3 of SEQ ID NO:140; and a light chain variable region that comprises an LCDR1 of SEQ ID NO:59, an LCDR2 of SEQ ID NO:39, and an LCDR3 of SEQ ID NO:118;
k. a heavy chain variable region that comprises an HCDR1 of SEQ ID NO:153, an HCDR2 of SEQ ID NO:154, and an HCDR3 of SEQ ID NO:155; and a light chain variable region that comprises an LCDR1 of SEQ ID NO:166, an LCDR2 of SEQ ID NO:167, and an LCDR3 of SEQ ID NO:168;
l. a heavy chain variable region that comprises an HCDR1 of SEQ ID NO:178, an HCDR2 of SEQ ID NO:179, and an HCDR3 of SEQ ID NO:180; and a light chain variable region that comprises an LCDR1 of SEQ ID NO:191, an LCDR2 of SEQ ID NO:192, and an LCDR3 of SEQ ID NO:193;
m. a heavy chain variable region that comprises an HCDR1 of SEQ ID NO:203, an HCDR2 of SEQ ID NO:204, and an HCDR3 of SEQ ID NO:205; and a light chain variable region that comprises an LCDR1 of SEQ ID NO:216, an LCDR2 of SEQ ID NO:217, and an LCDR3 of SEQ ID NO:218;
n. a heavy chain variable region that comprises an HCDR1 of SEQ ID NO:227, an HCDR2 of SEQ ID NO:228, and an HCDR3 of SEQ ID NO:229; and a light chain variable region that comprises an LCDR1 of SEQ ID NO:216, an LCDR2 of SEQ ID NO:217, and an LCDR3 of SEQ ID NO:238;
o. a heavy chain variable region that comprises an HCDR1 of SEQ ID NO:244, an HCDR2 of SEQ ID NO:26, and an HCDR3 of SEQ ID NO:245; and a light chain variable region that comprises an LCDR1 of SEQ ID NO:253, an LCDR2 of SEQ ID NO:254, and an LCDR3 of SEQ ID NO:255;
p. a heavy chain variable region that comprises an HCDR1 of SEQ ID NO:264, an HCDR2 of SEQ ID NO:265, and an HCDR3 of SEQ ID NO:266; and a light chain variable region that comprises an LCDR1 of SEQ ID NO:277, an LCDR2 of SEQ ID NO:278, and an LCDR3 of SEQ ID NO:279;
q. a heavy chain variable region that comprises an HCDR1 of SEQ ID NO:264, an HCDR2 of SEQ ID NO:265, and an HCDR3 of SEQ ID NO:296; and a light chain variable region that comprises an LCDR1 of SEQ ID NO:277, an LCDR2 of SEQ ID NO:278, and an LCDR3 of SEQ ID NO:279.

The invention provides an antibody or antigen binding fragment thereof that binds DC-SIGN comprising:
a. A heavy chain variable region (VH) comprising the amino acid sequence of SEQ ID NO:10, and a light chain variable region (VL) comprising the amino acid sequence of SEQ ID NO:21;
b. A heavy chain variable region (VH) comprising the amino acid sequence of SEQ ID NO:34, and a light chain variable region (VL) comprising the amino acid sequence of SEQ ID NO:45;
c. A heavy chain variable region (VH) comprising the amino acid sequence of SEQ ID NO:55, and a light chain variable region (VL) comprising the amino acid sequence of SEQ ID NO:64;
d. A heavy chain variable region (VH) comprising the amino acid sequence of SEQ ID NO:34, and a light chain variable region (VL) comprising the amino acid sequence of SEQ ID NO:70;
e. A heavy chain variable region (VH) comprising the amino acid sequence of SEQ ID NO:78, and a light chain variable region (VL) comprising the amino acid sequence of SEQ ID NO:84;
f. A heavy chain variable region (VH) comprising the amino acid sequence of SEQ ID NO:90, and a light chain variable region (VL) comprising the amino acid sequence of SEQ ID NO:99;
g. A heavy chain variable region (VH) comprising the amino acid sequence of SEQ ID NO:103, and a light chain variable region (VL) comprising the amino acid sequence of SEQ ID NO:107;
h. A heavy chain variable region (VH) comprising the amino acid sequence of SEQ ID NO: 114, and a light chain variable region (VL) comprising the amino acid sequence of SEQ ID NO:120;
i. A heavy chain variable region (VH) comprising the amino acid sequence of SEQ ID NO:55, and a light chain variable region (VL) comprising the amino acid sequence of SEQ ID NO:126;
j. A heavy chain variable region (VH) comprising the amino acid sequence of SEQ ID NO:78, and a light chain variable region (VL) comprising the amino acid sequence of SEQ ID NO:130;
k. A heavy chain variable region (VH) comprising the amino acid sequence of SEQ ID NO:90, and a light chain variable region (VL) comprising the amino acid sequence of SEQ ID NO:134;
l. A heavy chain variable region (VH) comprising the amino acid sequence of SEQ ID NO:145, and a light chain variable region (VL) comprising the amino acid sequence of SEQ ID NO:149;
m. A heavy chain variable region (VH) comprising the amino acid sequence of SEQ ID NO:162, and a light chain variable region (VL) comprising the amino acid sequence of SEQ ID NO:174;
n. A heavy chain variable region (VH) comprising the amino acid sequence of SEQ ID NO:187, and a light chain variable region (VL) comprising the amino acid sequence of SEQ ID NO:199;
o. A heavy chain variable region (VH) comprising the amino acid sequence of SEQ ID NO:212, and a light chain variable region (VL) comprising the amino acid sequence of SEQ ID NO:223;
p. A heavy chain variable region (VH) comprising the amino acid sequence of SEQ ID NO:234, and a light chain variable region (VL) comprising the amino acid sequence of SEQ ID NO:240;
q. A heavy chain variable region (VH) comprising the amino acid sequence of SEQ ID NO:249, and a light chain variable region (VL) comprising the amino acid sequence of SEQ ID NO:260;
r. A heavy chain variable region (VH) comprising the amino acid sequence of SEQ ID NO:273, and a light chain variable region (VL) comprising the amino acid sequence of SEQ ID NO:284;
s. A heavy chain variable region (VH) comprising the amino acid sequence of SEQ ID NO:288, and a light chain variable region (VL) comprising the amino acid sequence of SEQ ID NO:292; or
t. A heavy chain variable region (VH) comprising the amino acid sequence of SEQ ID NO:298, and a light chain variable region (VL) comprising the amino acid sequence of SEQ ID NO:284.

The invention provides an antibody or antigen binding fragment thereof that binds DC-SIGN comprising:
a. A heavy chain comprising the amino acid sequence of SEQ ID NO:12, and a light chain comprising the amino acid sequence of SEQ ID NO:23;
b. A heavy chain comprising the amino acid sequence of SEQ ID NO:36, and a light chain comprising the amino acid sequence of SEQ ID NO:47;
c. A heavy chain comprising the amino acid sequence of SEQ ID NO:57, and a light chain comprising the amino acid sequence of SEQ ID NO:66;
d. A heavy chain comprising the amino acid sequence of SEQ ID NO:36, and a light chain comprising the amino acid sequence of SEQ ID NO:72;
e. A heavy chain comprising the amino acid sequence of SEQ ID NO:80, and a light chain comprising the amino acid sequence of SEQ ID NO:86;
f. A heavy chain comprising the amino acid sequence of SEQ ID NO:92, and a light chain comprising the amino acid sequence of SEQ ID NO:101;
g. A heavy chain comprising the amino acid sequence of SEQ ID NO:105, and a light chain comprising the amino acid sequence of SEQ ID NO:109;
h. A heavy chain comprising the amino acid sequence of SEQ ID NO: 116, and a light chain comprising the amino acid sequence of SEQ ID NO:122;
i. A heavy chain comprising the amino acid sequence of SEQ ID NO:57, and a light chain comprising the amino acid sequence of SEQ ID NO:128;
j. A heavy chain comprising the amino acid sequence of SEQ ID NO:80, and a light chain comprising the amino acid sequence of SEQ ID NO:132;
k. A heavy chain comprising the amino acid sequence of SEQ ID NO:92, and a light chain comprising the amino acid sequence of SEQ ID NO:136;
l. A heavy chain comprising the amino acid sequence of SEQ ID NO:147, and a light chain comprising the amino acid sequence of SEQ ID NO:151;
m. A heavy chain comprising the amino acid sequence of SEQ ID NO:164, and a light chain comprising the amino acid sequence of SEQ ID NO:176;
n. A heavy chain comprising the amino acid sequence of SEQ ID NO:189, and a light chain comprising the amino acid sequence of SEQ ID NO:201;
o. A heavy chain comprising the amino acid sequence of SEQ ID NO:214, and a light chain comprising the amino acid sequence of SEQ ID NO:225;
p. A heavy chain comprising the amino acid sequence of SEQ ID NO:236, and a light chain comprising the amino acid sequence of SEQ ID NO:242;
q. A heavy chain comprising the amino acid sequence of SEQ ID NO:251, and a light chain comprising the amino acid sequence of SEQ ID NO:262;
r. A heavy chain) comprising the amino acid sequence of SEQ ID NO:275, and a light chain comprising the amino acid sequence of SEQ ID NO:286;
s. A heavy chain comprising the amino acid sequence of SEQ ID NO:290, and a light chain comprising the amino acid sequence of SEQ ID NO:294; or
t. A heavy chain comprising the amino acid sequence of SEQ ID NO:300, and a light chain comprising the amino acid sequence of SEQ ID NO:286.

The invention provides antibody conjugates comprising immunomodulators, pharmaceutically acceptable salts thereof, pharmaceutical compositions thereof and combinations thereof, which are useful for the treatment of diseases, in particular, cancer. The invention provides antibody conjugates comprising toll-like receptor agonists, pharmaceutically acceptable salts thereof, pharmaceutical compositions thereof and combinations thereof, which are useful for the treatment of diseases, in particular, cancer. The invention provides antibody conjugates comprising RIG-1 agonists, pharmaceutically acceptable salts thereof, pharmaceutical compositions thereof and combinations thereof, which are useful for the treatment of diseases, in particular, cancer. The invention further provides methods of treating, preventing, or ameliorating cancer comprising administering to a subject in need thereof an effective amount of an antibody conjugate of the invention. The invention also provides compounds comprising TLR7 agonists and a linker which are useful to conjugate to an anti-DC-SIGN antibody and thereby make the immunostimmulatory conjugates of the invention. The invention also provides compounds comprising TLR7 agonists and a linker which are useful to conjugate to an anti-DC-SIGN antibody and thereby make the immunostimmulatory conjugates of the invention.

Various embodiments of the invention are described herein.

In one aspect of the invention are conjugates comprising an anti-DC-SIGN antibody disclosed herein or an antigen binding fragment thereof, coupled to drog moiety (D) via a linker (L), wherein the linker optionally comprises one or more cleavage or non-cleavable elements.

In some embodiments, the conjugate comprises Formula (III):

Ab-(L-(D)ₘ)ₙ (Formula (III))
wherein:
   Ab is an anti-DC-SIGN antibody or a functional fragment thereof;
   L is a linker comprising one or more cleavage or non-cleavable elements;
   D is the drug moiety;
   m is an integer from 1 to 8; and
   n is an integer from 1 to 20.

In some embodiments, the drug moiety is an immunostimulatory molecule, a cytotoxic molecule, a radionuclide, etc. In some embodiments, the immunostimulatory molecule is a small molecule compound, a nucleic acid molecule, a polypeptide, or a combination thereof. In some embodiments, the immunostimulatory molecule is a dendritic cell stimulating compound, for example, a DEC-205 agonist, FLT3 ligand, granulocyte macrophage colony-stimulating factor (GM-CSF), an agonist of a Toll-like receptor (TLR) (e.g., TLR1, TLR2, TLR3, TLR4, TLR5, TLR6, TLR7, TLR8, TLR9, TLR10), RIG-I, MDA-5, LGP2, a C-type lectin receptor agonist, NOD1, NOD2, costimulatory compounds such as IL-15 or agonists of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand. In some embodiments, the immunostimulatory molecule is an agonist of TLR7. In some embodiments, the immunostimulatory molecule is an agonist of RIG-I.

In one aspect of the invention are compounds having the structure of Formula (I), and the pharmaceutically acceptable salts thereof, which are TLR7 agonists:

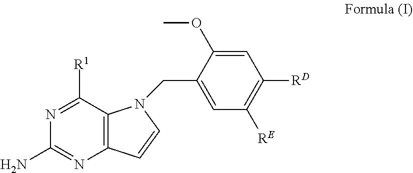

wherein:
R^{D} is

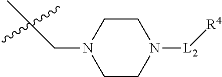

and R^{E} is H; or R^{E} is

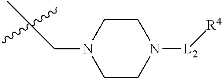

### and R^{D} is H;

R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴;
R₄ is

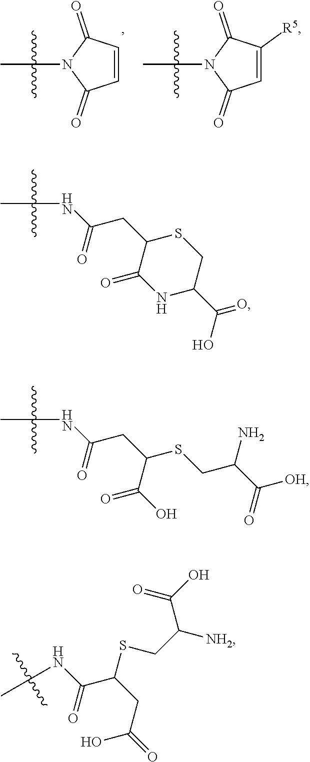

—ONH₂, —NH₂,

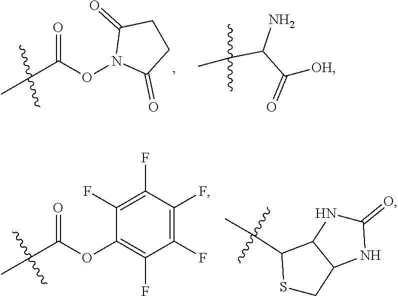 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

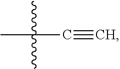 [SMILES: *C#C]

—NHC(═O)CH═CH₂, —SH, —SR⁷, —OH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

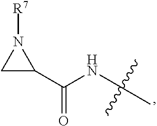

—CO₂H, —C(O)NHNH₂,

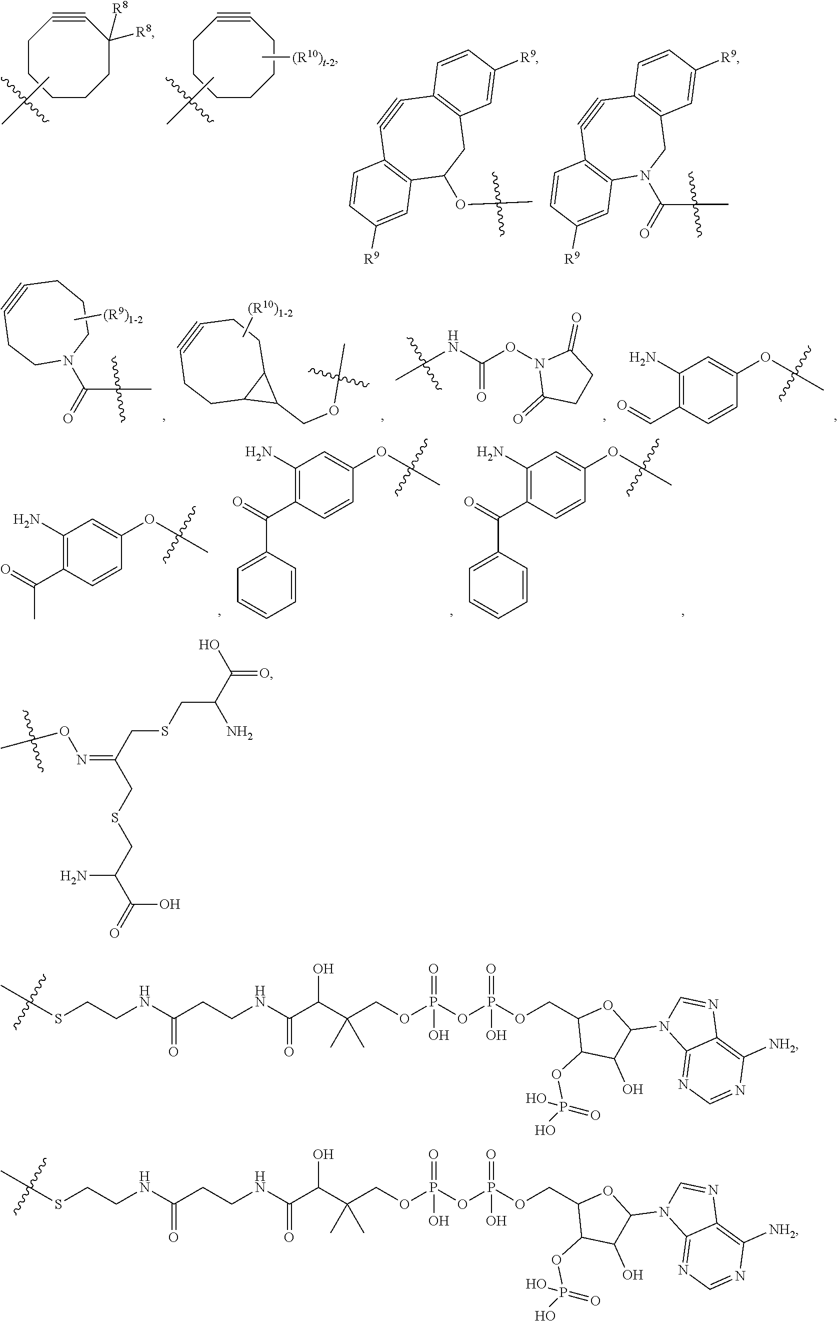

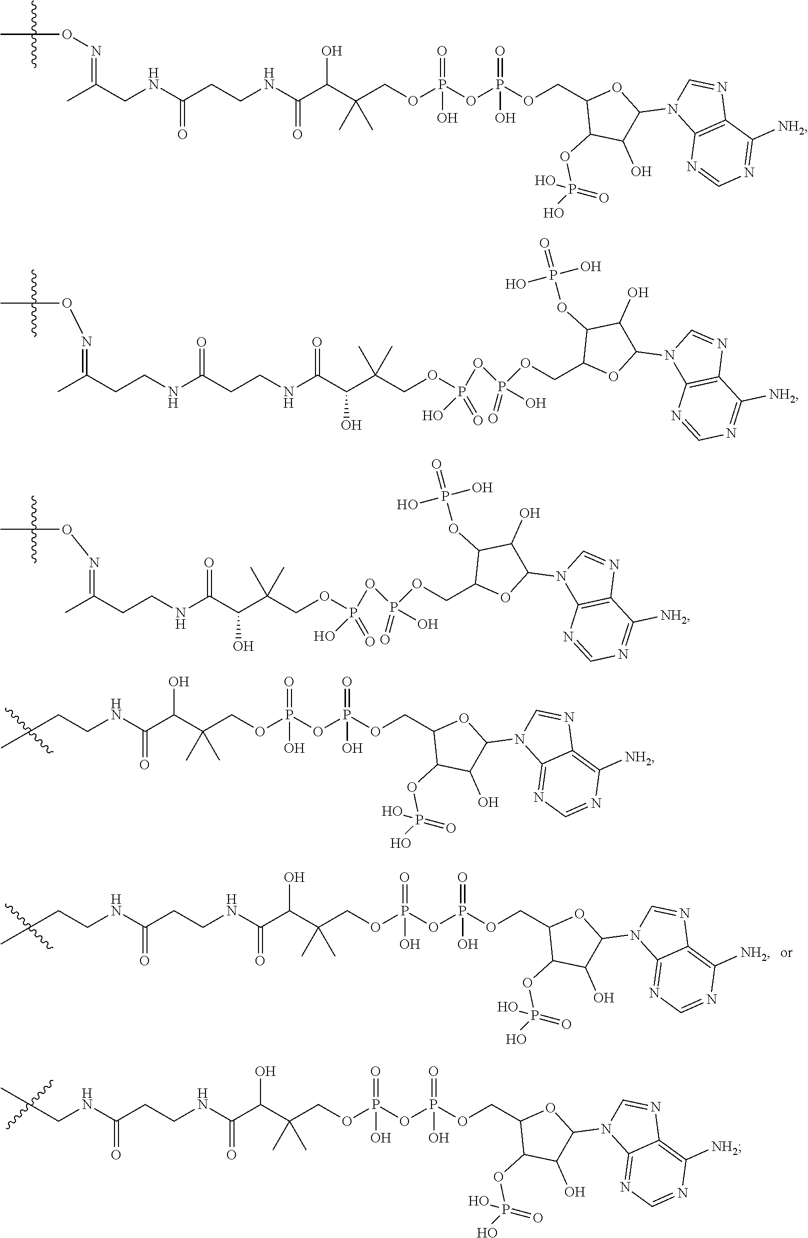

R⁵ is

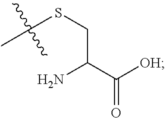 [SMILES: *SCC(N)C(=O)O]

X₁ is

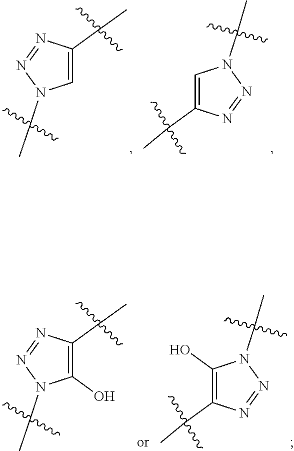 [SMILES: *c1cn(*)nn1 ; *c1cn(*)nn1 ; *c1nnn(*)c1O ; *c1nnn(*)c1O]

X₂ is

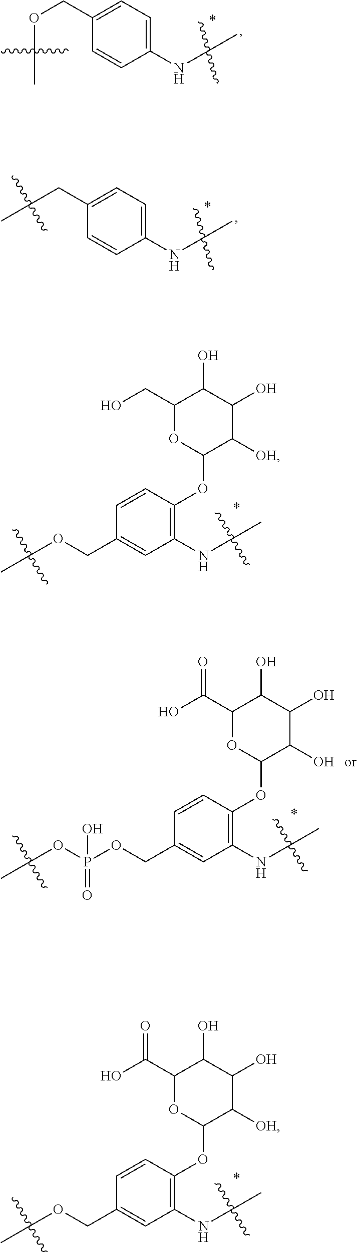 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

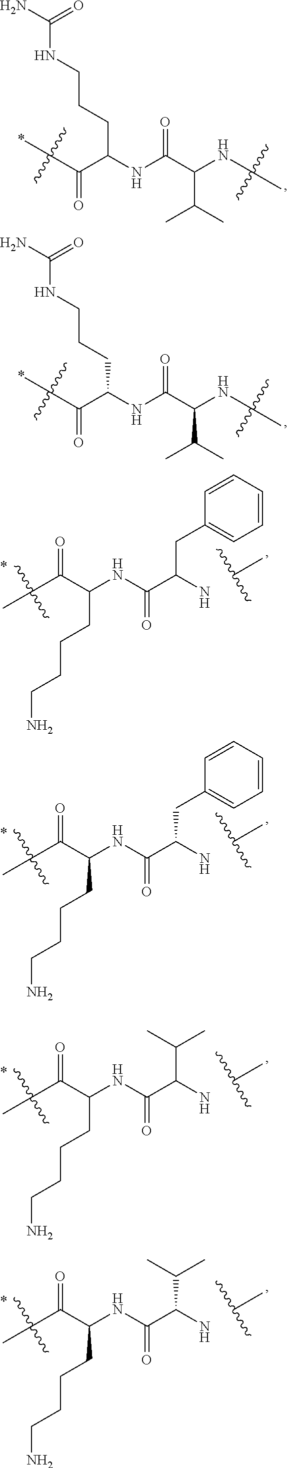

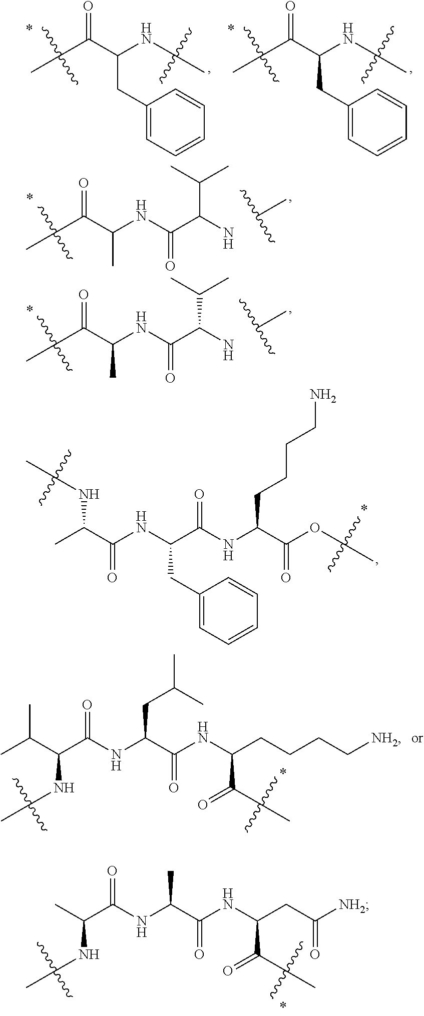

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and each t is independently selected from 1, 2, 3, 4, 5, 6, 7, 8, 9, 10, 11, 12, 13, 14, 15, 16, 17 and 18.

In one aspect of the invention are compounds having the structure of Formula (I), and the pharmaceutically acceptable salts thereof, which are TLR7 agonists:

wherein:

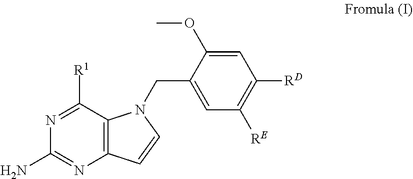

R^{D}

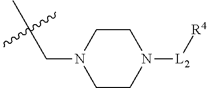

and R^{E} is H; or R^{E} is

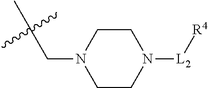

### and R^{D} is H;

R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴;
R⁴ is

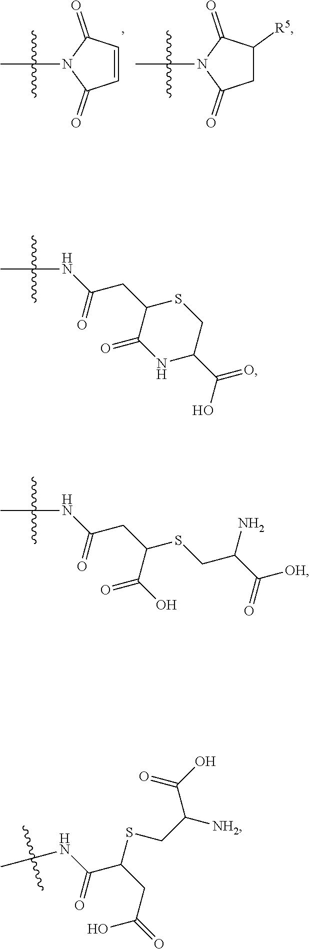

—ONH₂, —NH₂,

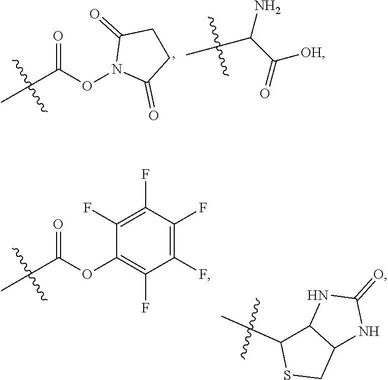 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

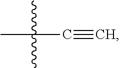 [SMILES: *C#C]

—NHC(═O)CH═CH₂, SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

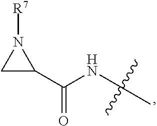

—CO₂H, —C(O)NHNH₂,

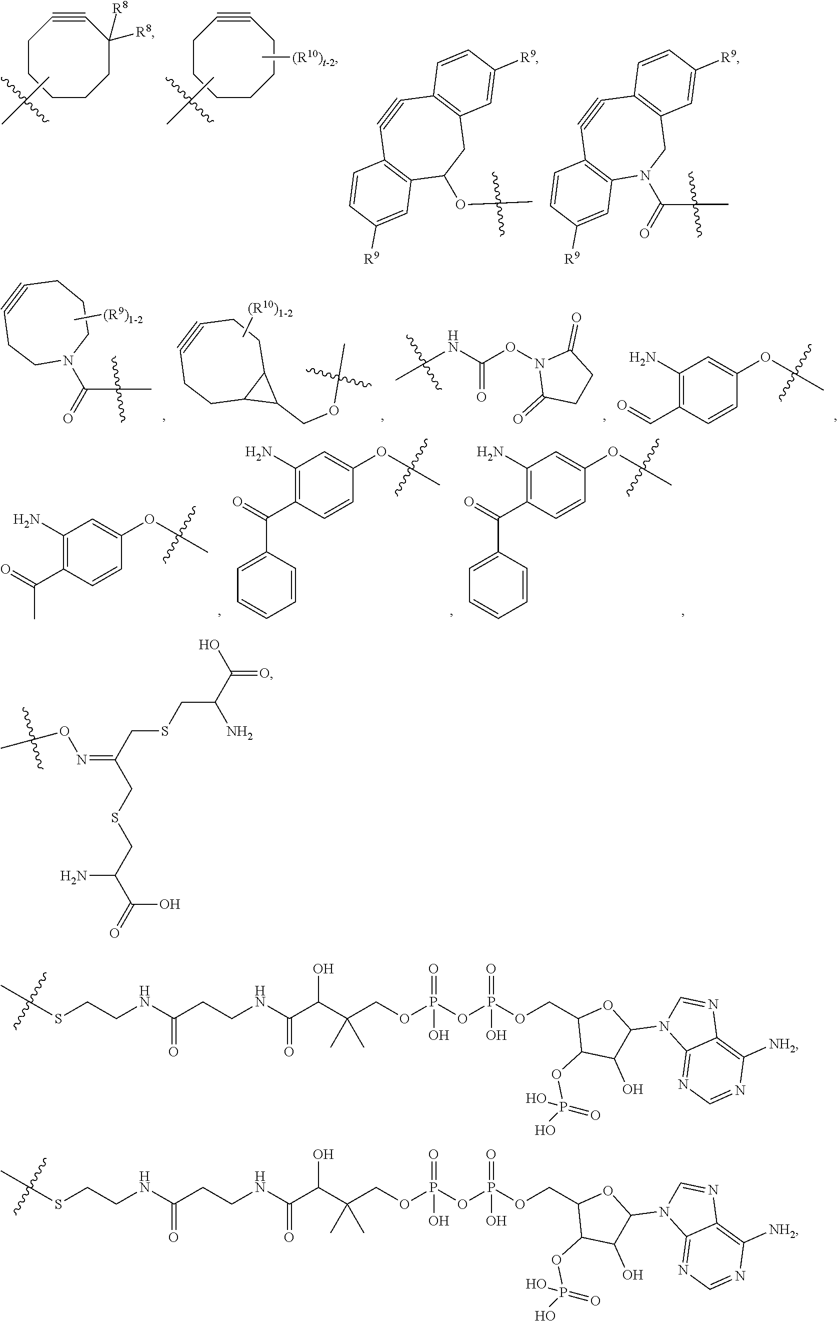

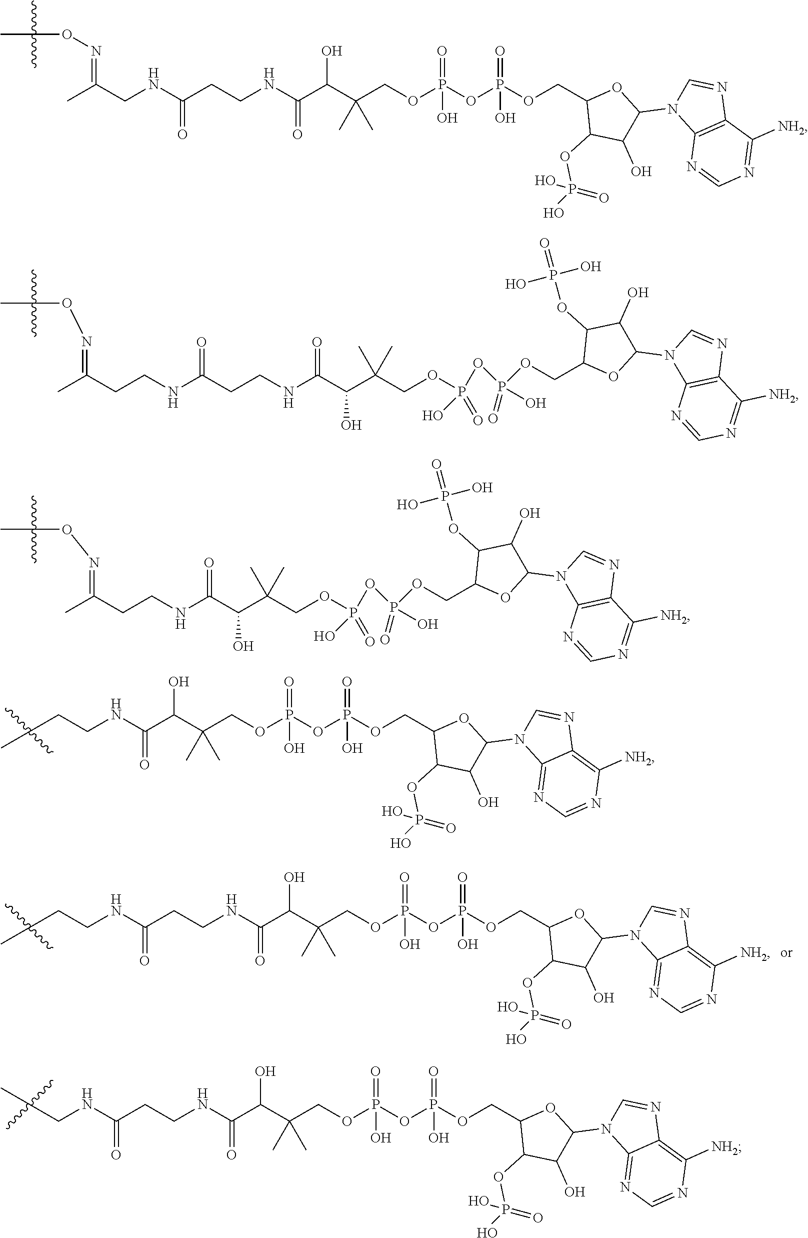

R⁵ is

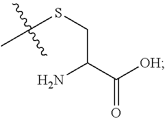 [SMILES: *SCC(N)C(=O)O]

X₁ is

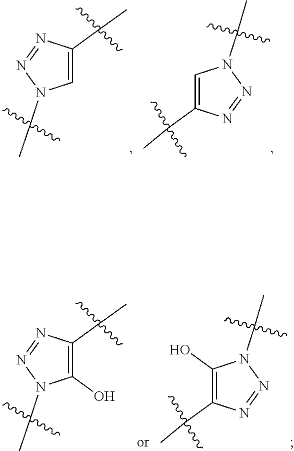 [SMILES: *c1cn(*)nn1 ; *c1cn(*)nn1 ; *c1nnn(*)c1O ; *c1nnn(*)c1O]

X₂ is

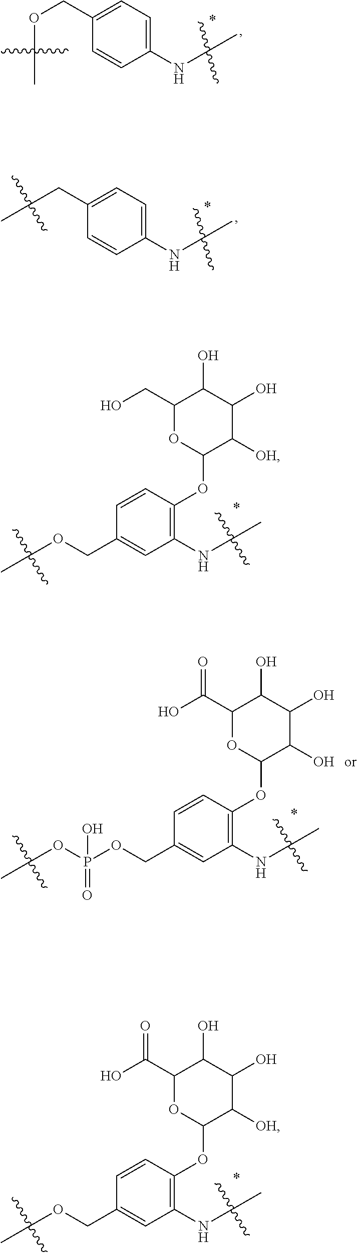 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃; X₃ is

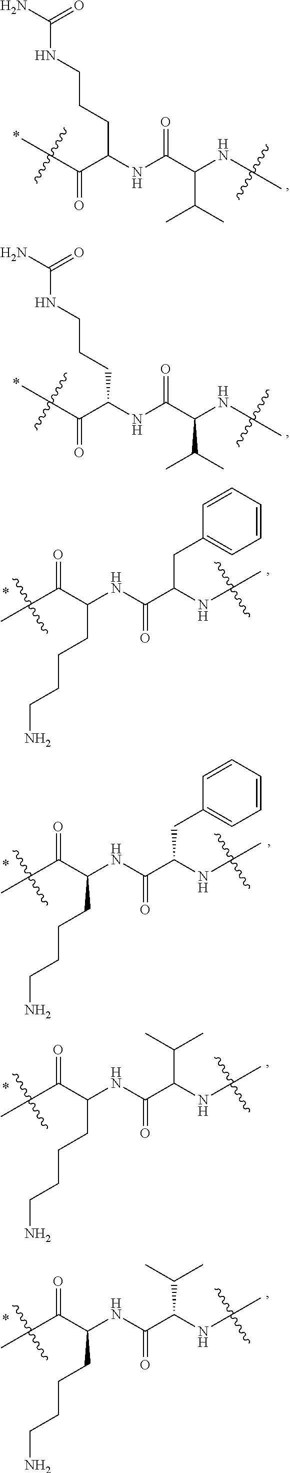

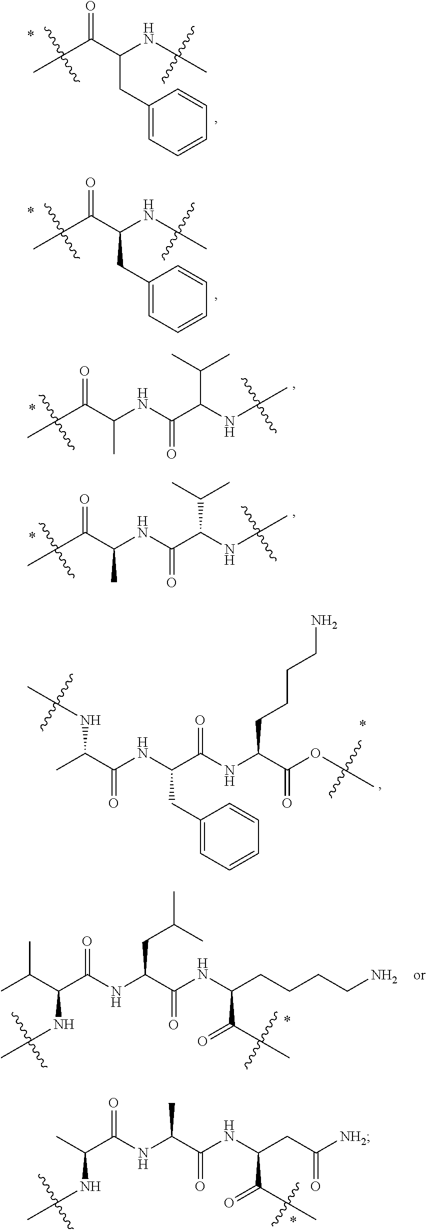

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

In one aspect of the invention are compounds of Formula (I) having the structure of Formula (Ia) or Formula (Ib), and the pharmaceutically acceptable salts thereof:

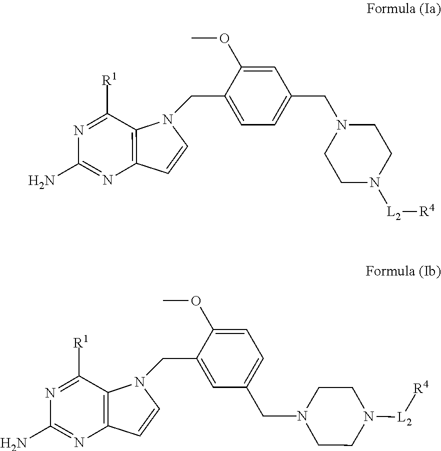

wherein:
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ**-, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴;
R₄ is

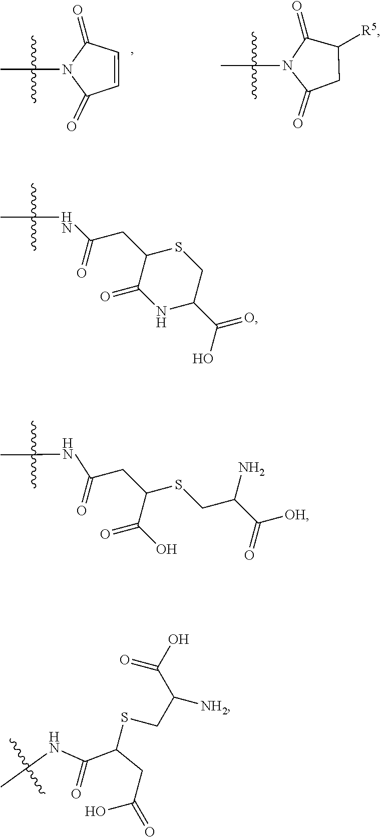

—ONH₂, —NH₂,

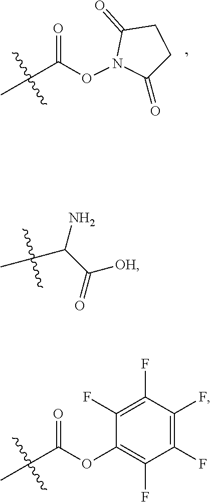 [SMILES: *C(=O)ON1C(=O)CCC1=O ; *C(N)C(=O)O ; *C(=O)Oc1c(F)c(F)c(F)c(F)c1F]

—NHC(═O)CH═CH₂, —N₃,

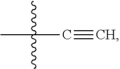 [SMILES: *C#C]

SH, —SR⁷, —OH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

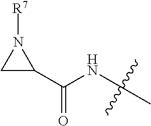

### or —CO₂H;

R⁵ is

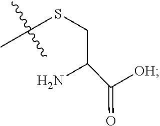 [SMILES: *SCC(N)C(=O)O]

X₁ is

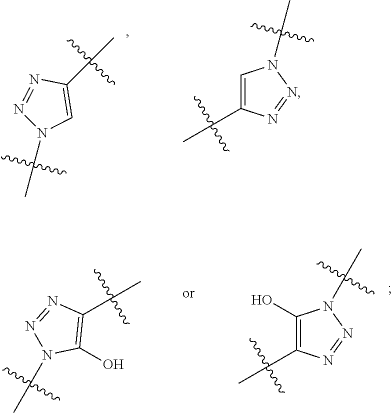 [SMILES: *c1cn(*)nn1 ; *c1cn(*)nn1 ; *c1nnn(*)c1O ; *c1nnn(*)c1O]

X₂ is

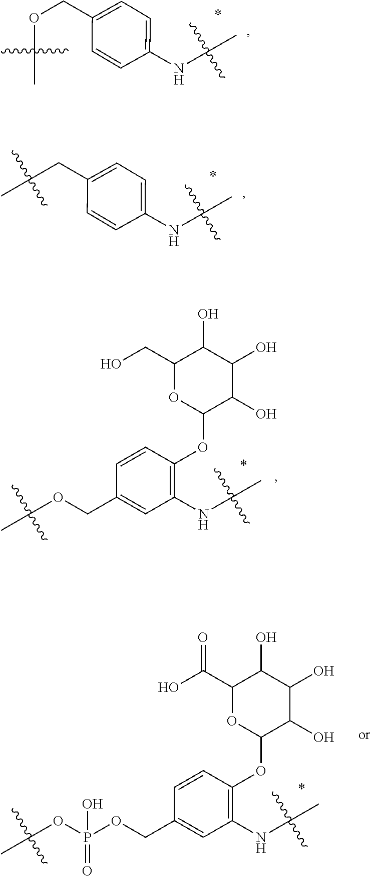 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O]

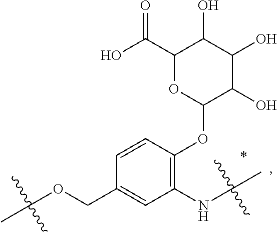 [SMILES: *Nc1cc(CO*)ccc1OC1OC(C(=O)O)C(O)C(O)C1O]

where the * of X₂ indicates the point of attachment to X₃;
X₃ is

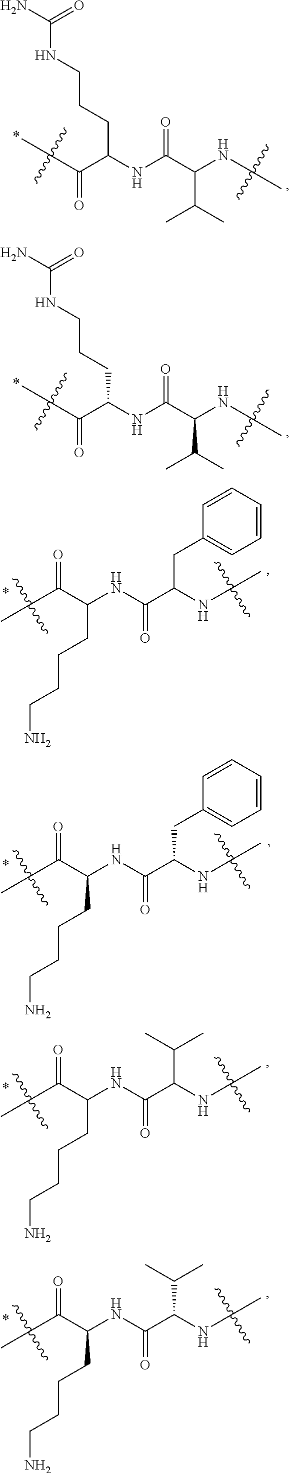

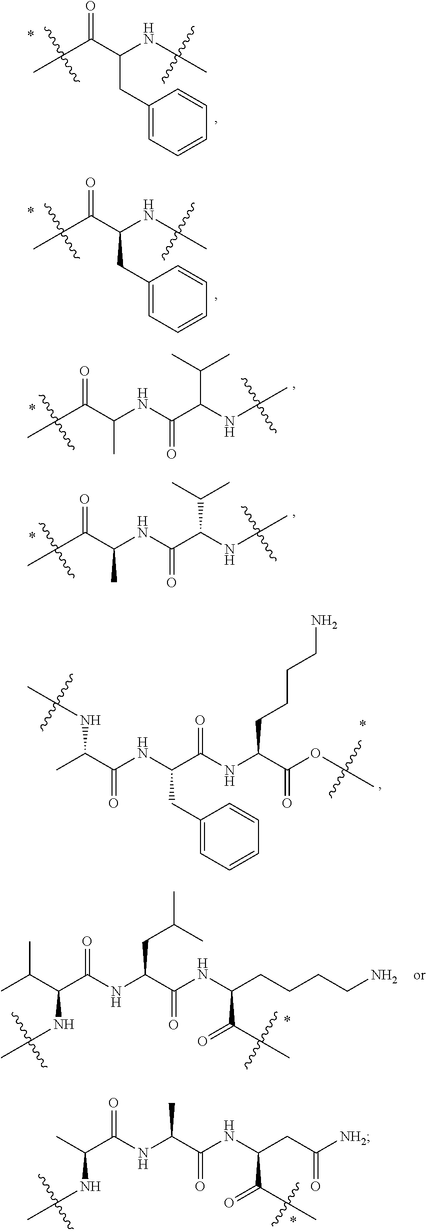

where the * of X₃ indicates the point of attachment to X₂;
R⁶ is 2-pyridyl or 4-pyridyl;
each R⁷ is independently selected from H and C₁-C₆alkyl;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

In one aspect of the invention are compounds of Formula (I) having the structure of Formula (Ia) or Formula (Ib), and the pharmaceutically acceptable salts thereof:

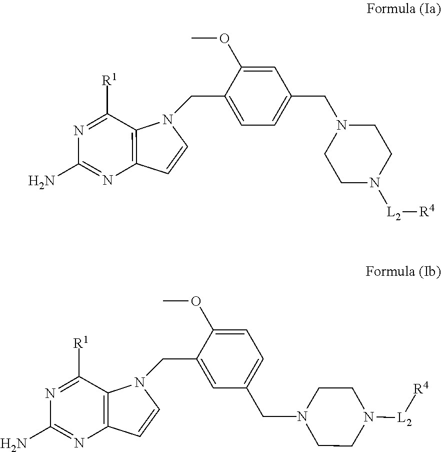

wherein:
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴;
R⁴ is

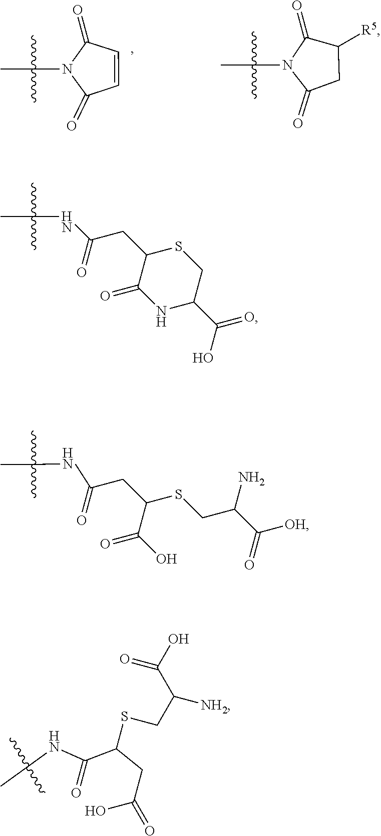

—ONH₂, —NH₂,

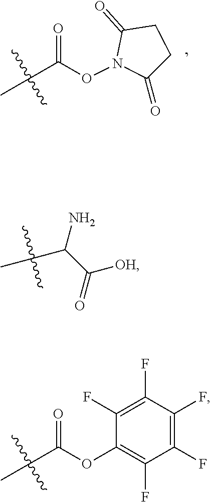 [SMILES: *C(=O)ON1C(=O)CCC1=O ; *C(N)C(=O)O ; *C(=O)Oc1c(F)c(F)c(F)c(F)c1F]

—NHC(═O)CH═CH₂, —N₃,

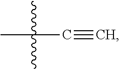 [SMILES: *C#C]

SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

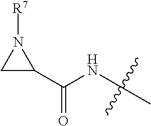

### or —CO₂H;

R⁵ is

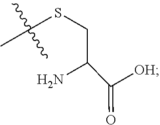 [SMILES: *SCC(N)C(=O)O]

X₁ is

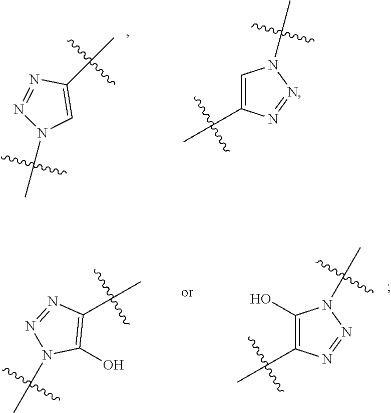 [SMILES: *c1cn(*)nn1 ; *c1cn(*)nn1 ; *c1nnn(*)c1O ; *c1nnn(*)c1O]

X₂ is

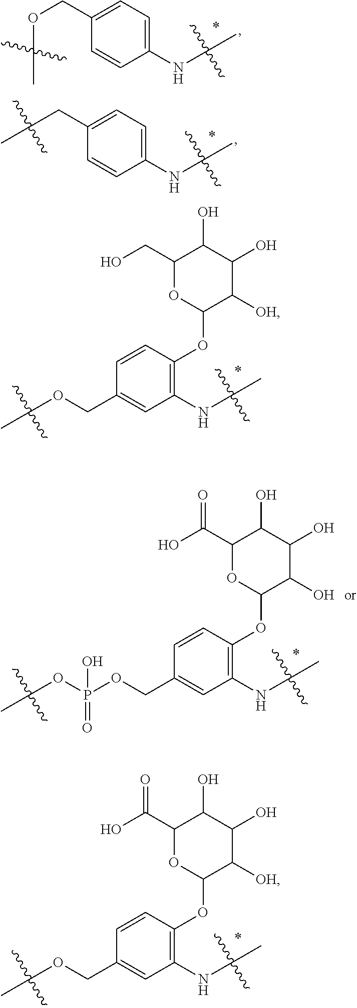

where the * of X₂ indicates the point of attachment to X₃;
X₃ is

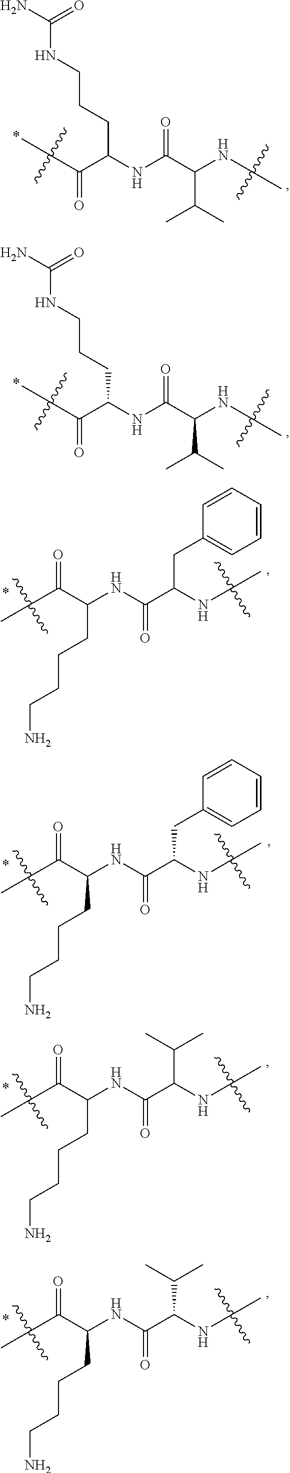

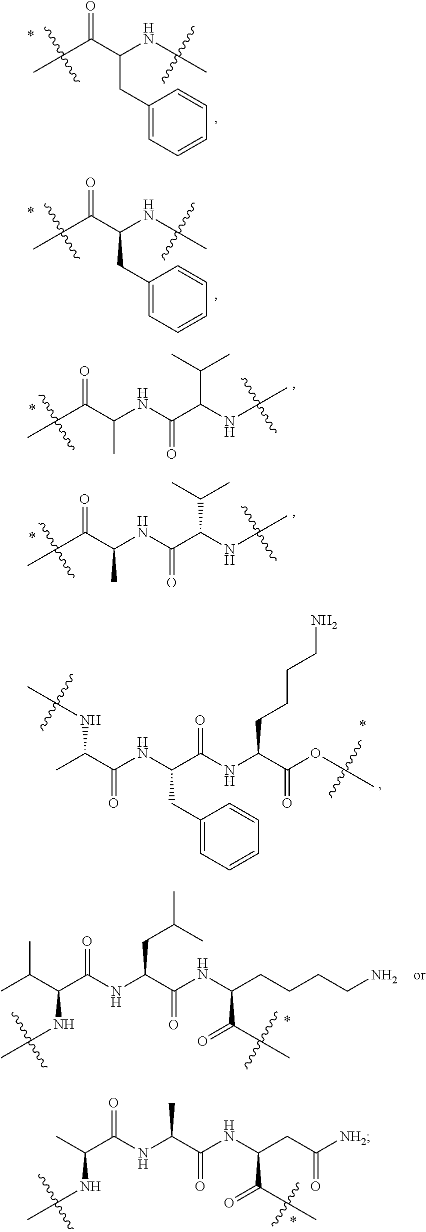

where the * of X₃ indicates the point of attachment to X₂;
R⁶ is 2-pyridyl or 4-pyridyl;
each R⁷ is independently selected from H and C₁-C₆alkyl;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

Another aspect of the invention are antibody conjugates having the structure of Formula (II), and the pharmaceutically acceptable salts thereof:

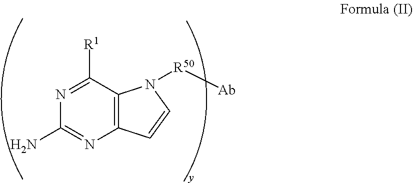

wherein:
R⁵⁰ is

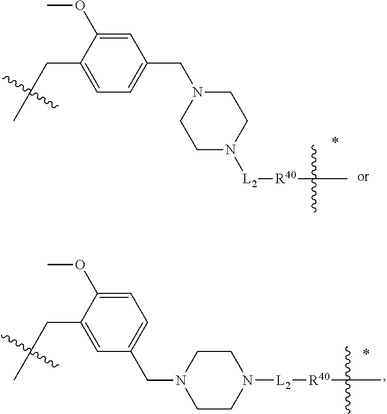

where the * indicates the point of attachment to Ab; Ab is an anti-DC-SIGN antibody or a functional fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ**-, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ**-, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ**-, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴⁰;

### R⁴⁰ is

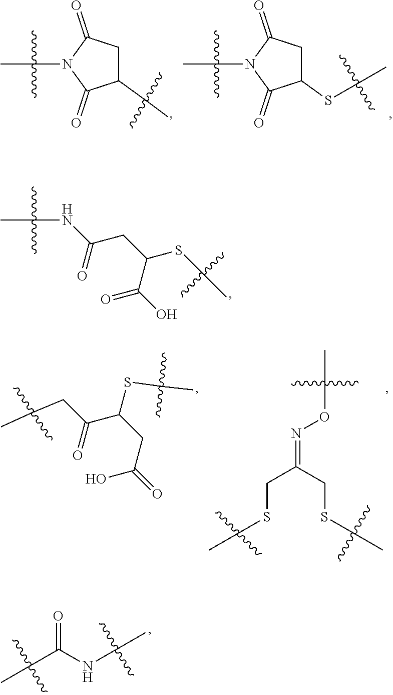 [SMILES: *C1CC(=O)N(*)C1=O ; *SC1CC(=O)N(*)C1=O ; *NC(=O)CC(S*)C(=O)O ; *CC(=O)C(CC(=O)O)S* ; *ON=C(CS*)CS* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂,

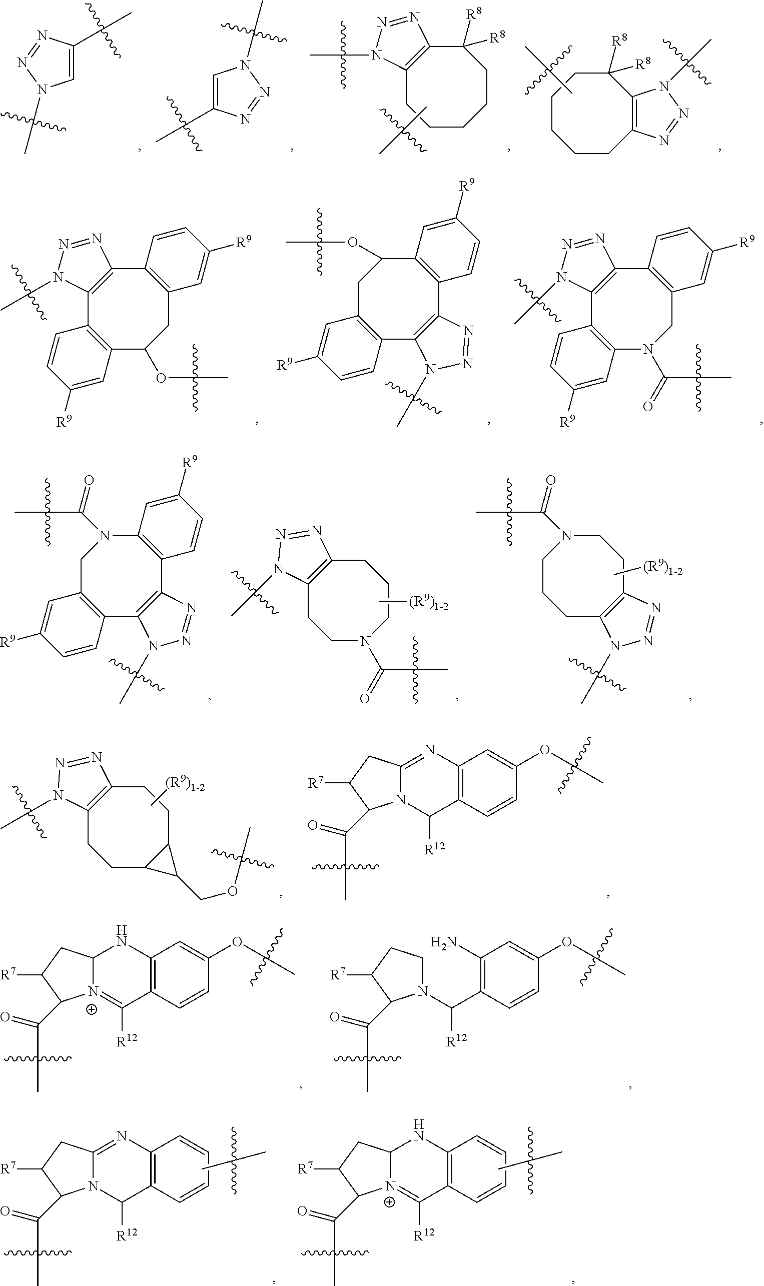

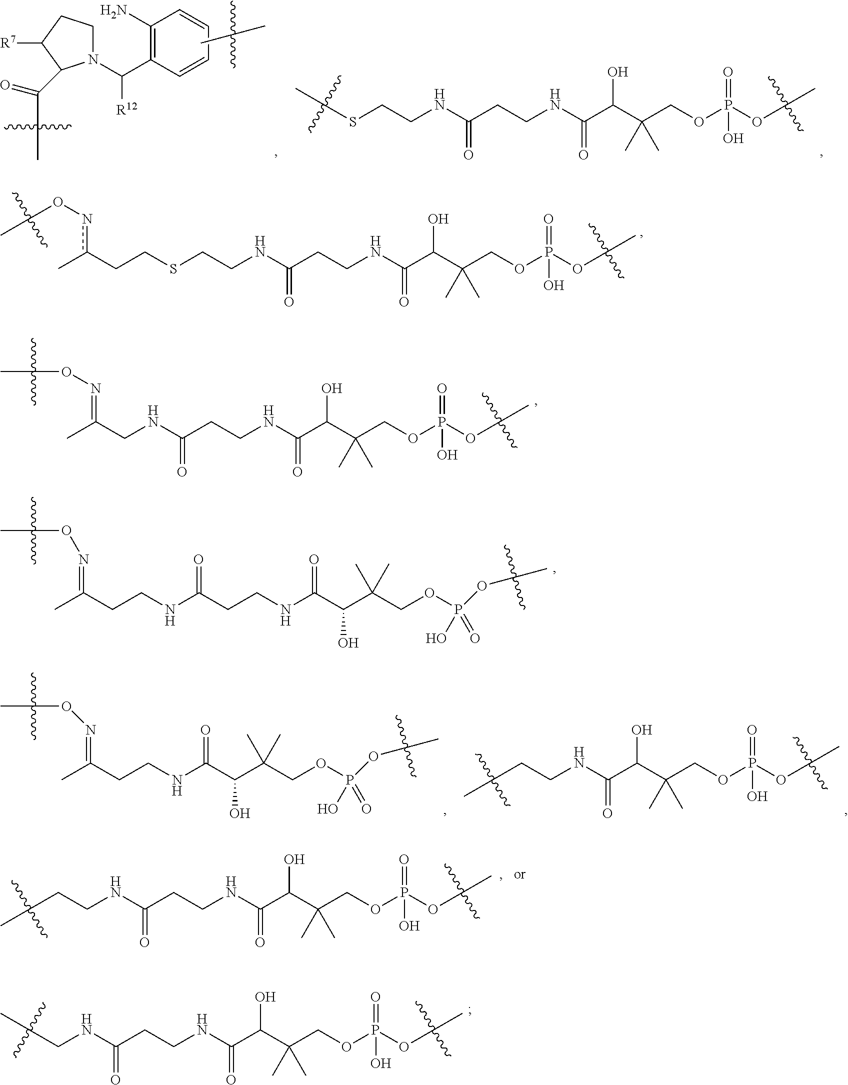

### X₁ is

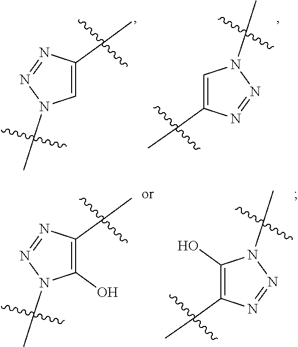 [SMILES: *c1cn(*)nn1 ; *c1cn(*)nn1 ; *c1nnn(*)c1O ; *c1nnn(*)c1O]

### X₂ is

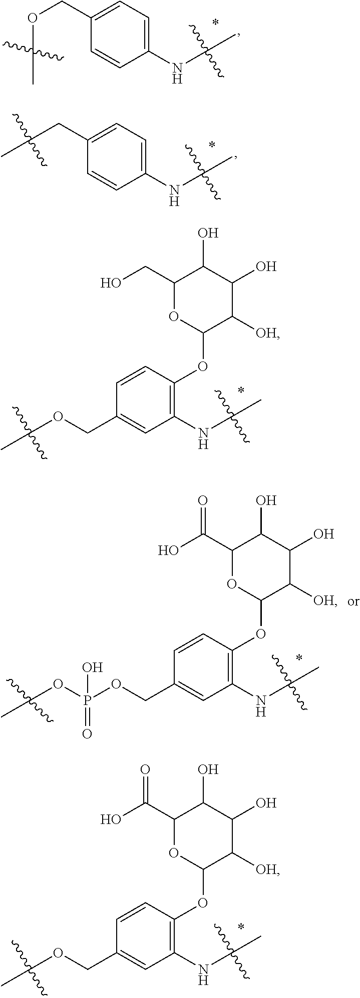 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;

### X₃ is

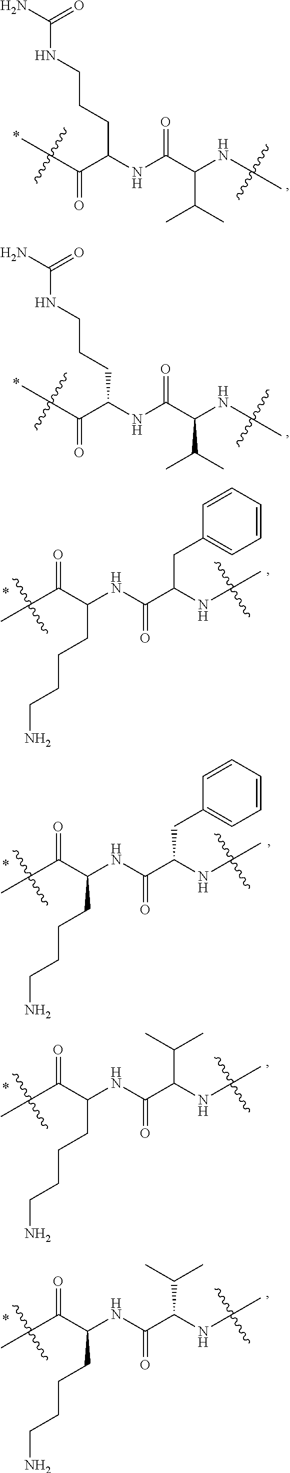

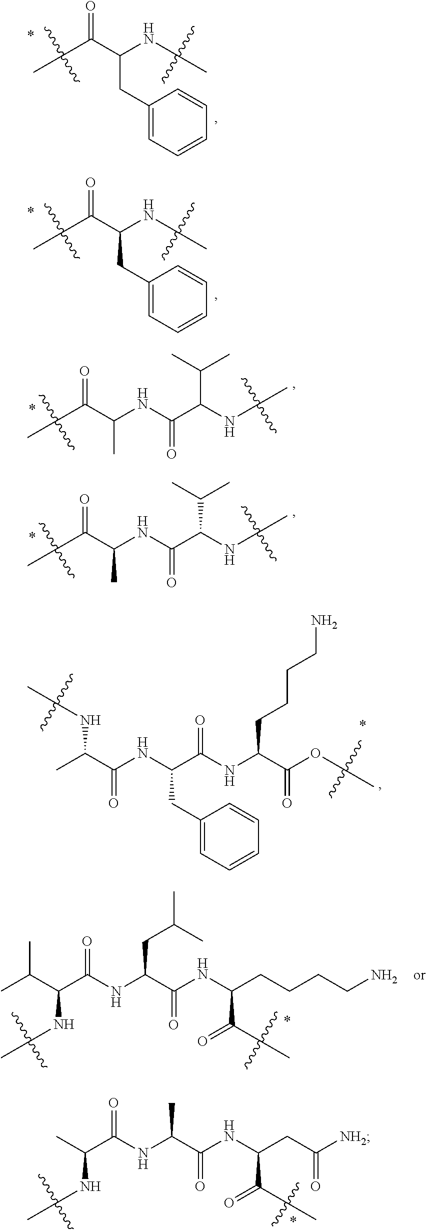

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl; each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Another aspect of the invention are antibody conjugates having the structure of Formula (II), and the pharmaceutically acceptable salts thereof:

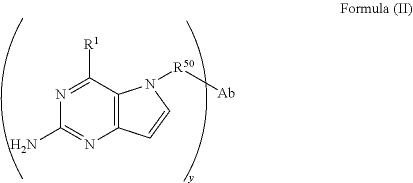

wherein:

### R⁵⁰ is

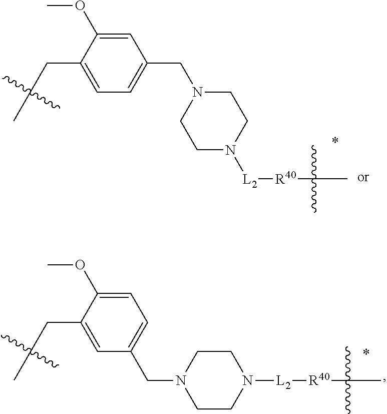

where the * indicates the point of attachment to Ab;
Ab is an anti-DC-SIGN antibody or a functional fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴⁰;

### R⁴⁰ is

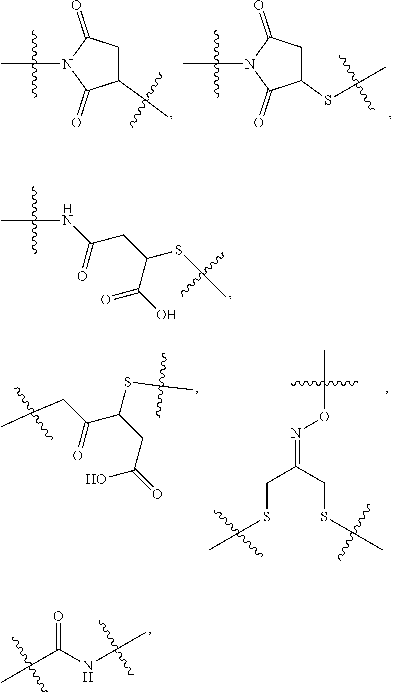 [SMILES: *C1CC(=O)N(*)C1=O ; *SC1CC(=O)N(*)C1=O ; *NC(=O)CC(S*)C(=O)O ; *CC(=O)C(CC(=O)O)S* ; *ON=C(CS*)CS* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂,

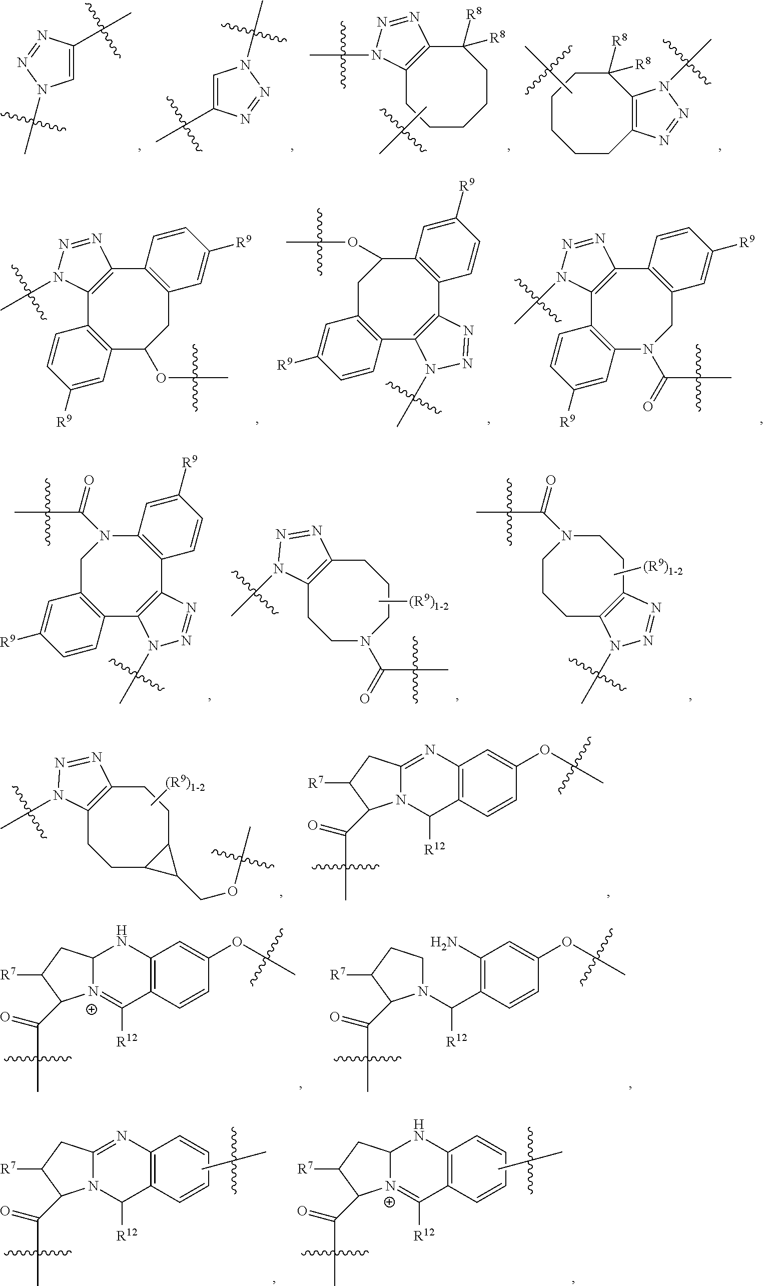

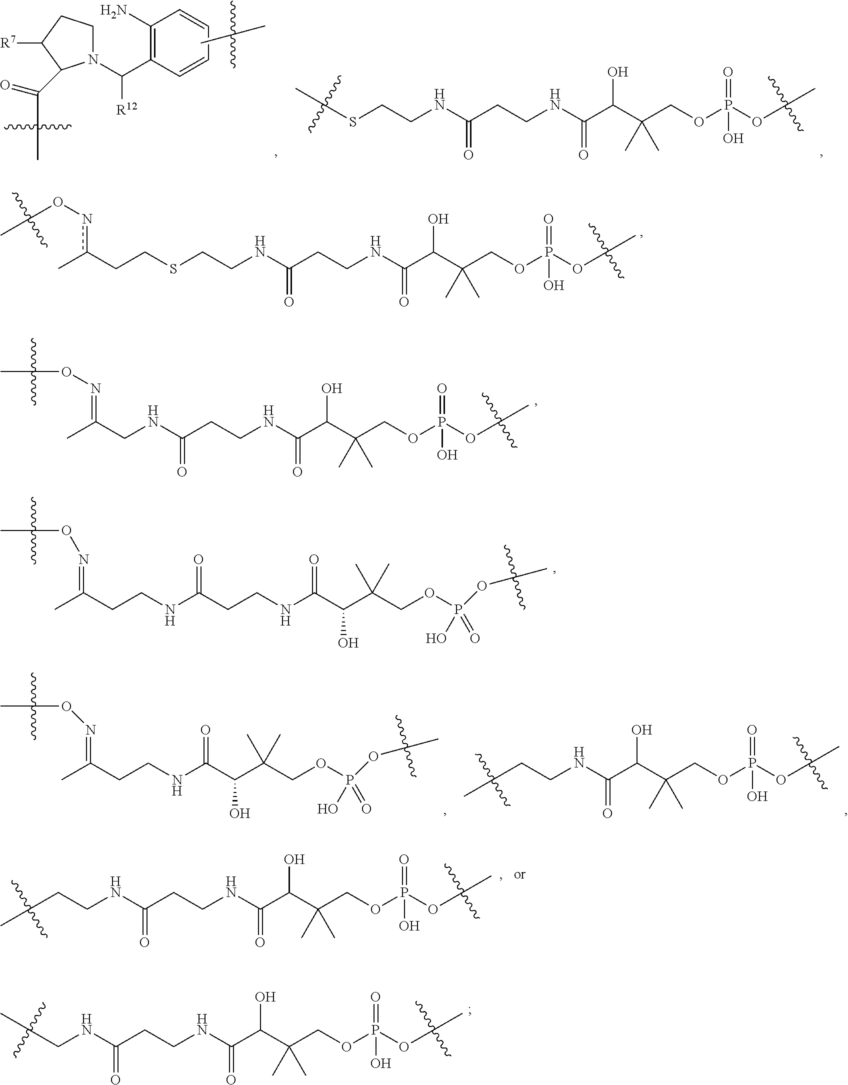

### X₁ is

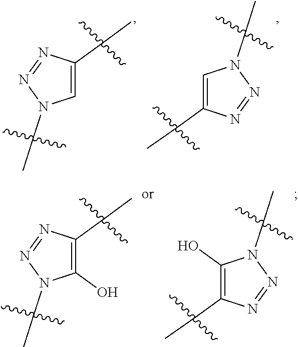 [SMILES: *c1cn(*)nn1 ; *c1cn(*)nn1 ; *c1nnn(*)c1O ; *c1nnn(*)c1O]

### X₂ is

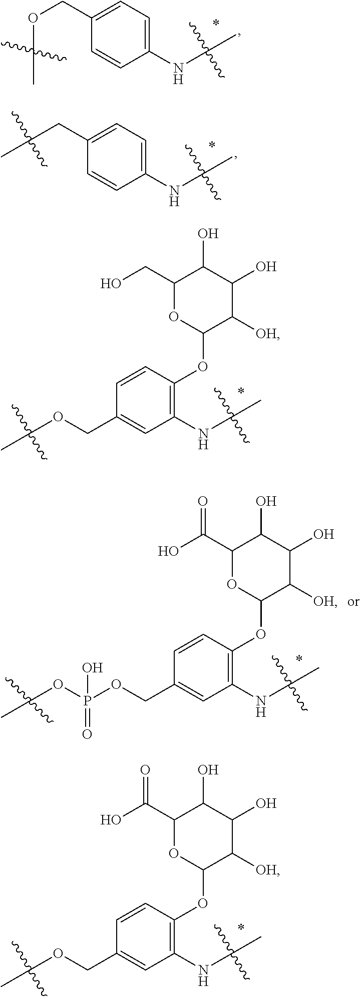 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;

### X₃ is

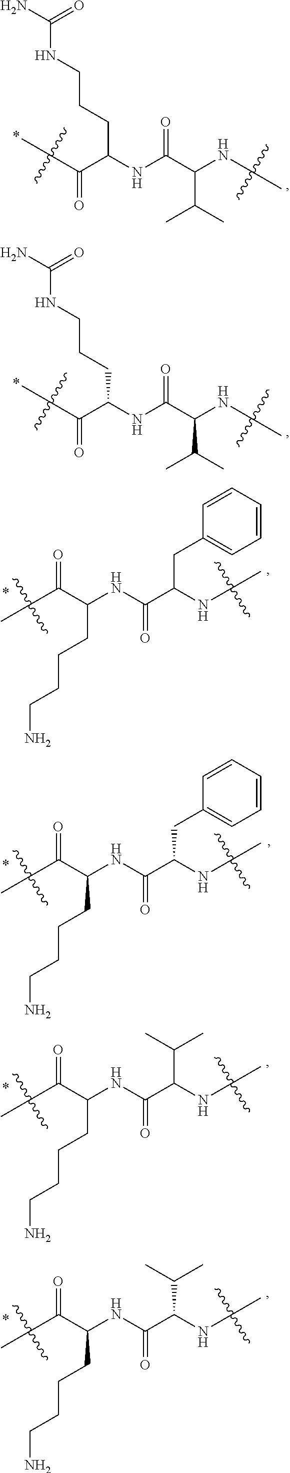

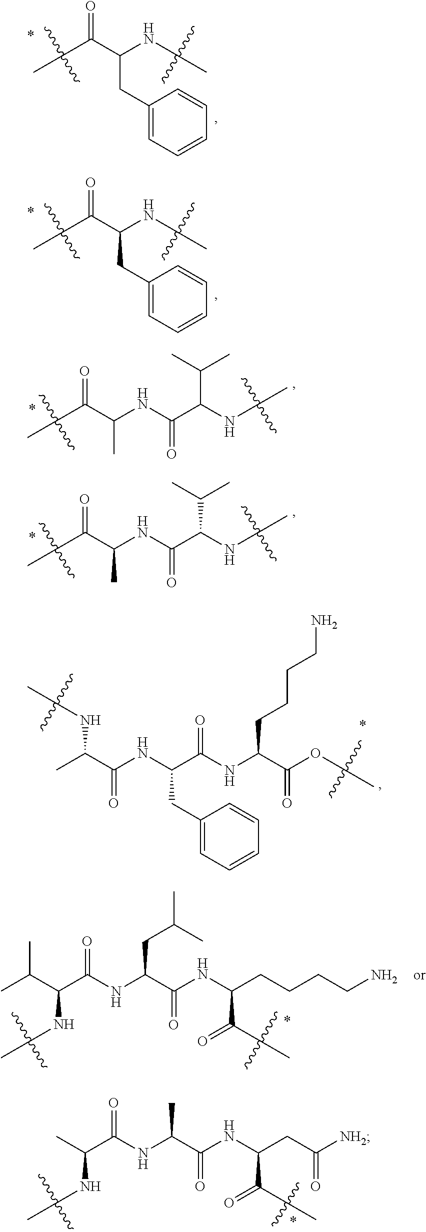

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Another aspect of the invention are antibody conjugates of Formula (II) having the structure of Formula (IIa) or Formula (IIb), and the pharmaceutically acceptable salts thereof:

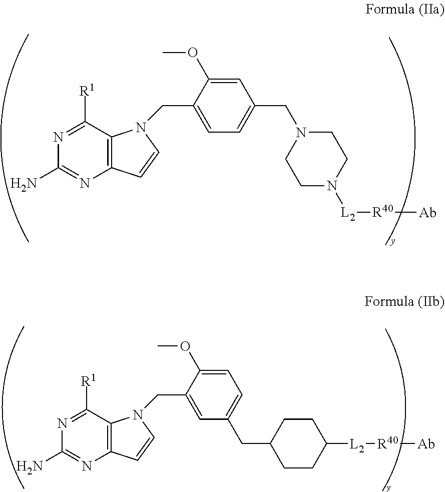

wherein:

Ab is an anti-DC-SIGN antibody or a functional fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴⁰;

### R⁴⁰ is

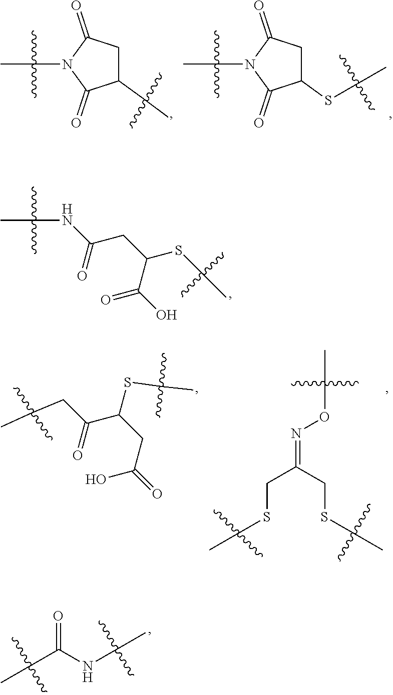 [SMILES: *C1CC(=O)N(*)C1=O ; *SC1CC(=O)N(*)C1=O ; *NC(=O)CC(S*)C(=O)O ; *CC(=O)C(CC(=O)O)S* ; *ON=C(CS*)CS* ; *NC(*)=O]

### or —S—;

### X₁ is

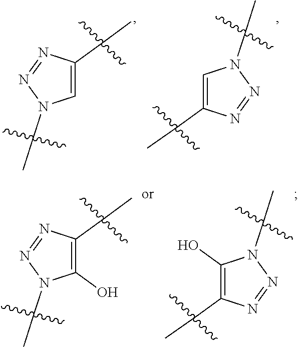 [SMILES: *c1cn(*)nn1 ; *c1cn(*)nn1 ; *c1nnn(*)c1O ; *c1nnn(*)c1O]

### X₂ is

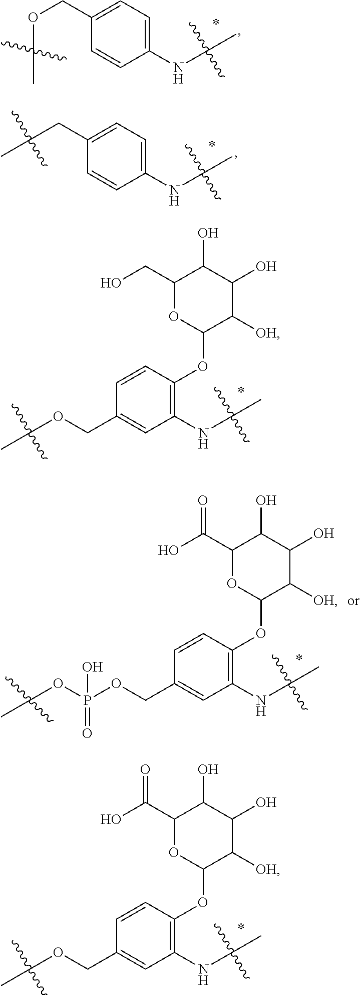 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;

### X₃ is

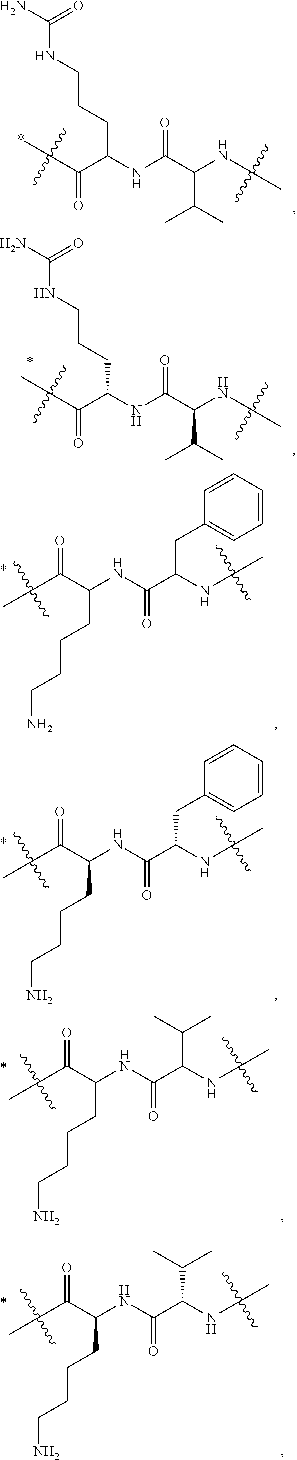

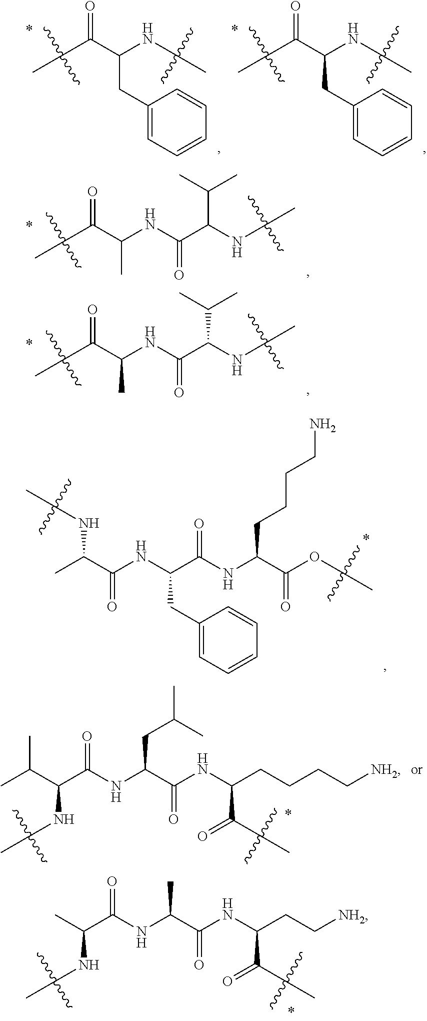

where the * of X₃ indicates the point of attachment to X₂;
each R⁷ is independently selected from H and C₁-C₆alkyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Another aspect of the invention are antibody conjugates of Formula (II) having the structure of Formula (IIa) or Formula (IIb), and the pharmaceutically acceptable salts thereof:

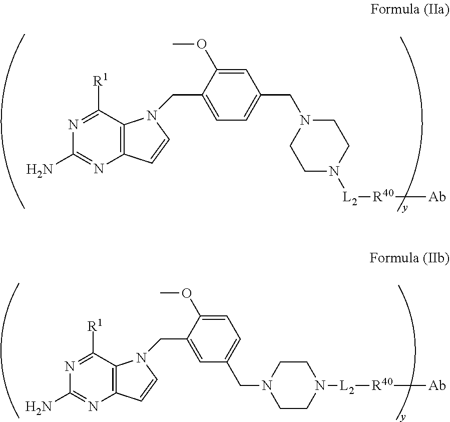

wherein:
Ab is an anti-DC-SIGN antibody or a functional fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ**-, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴⁰;
R⁴⁰ is

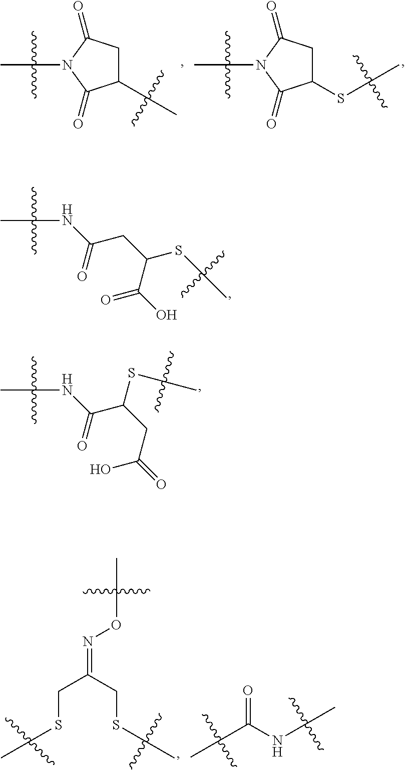 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

### or —S—;

X₁ is

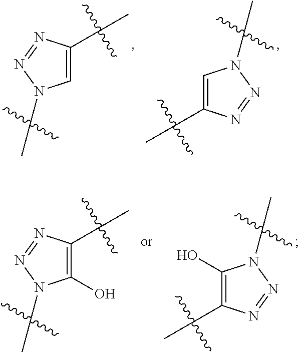 [SMILES: *c1cn(*)nn1 ; *c1cn(*)nn1 ; *c1nnn(*)c1O ; *c1nnn(*)c1O]

X₂ is

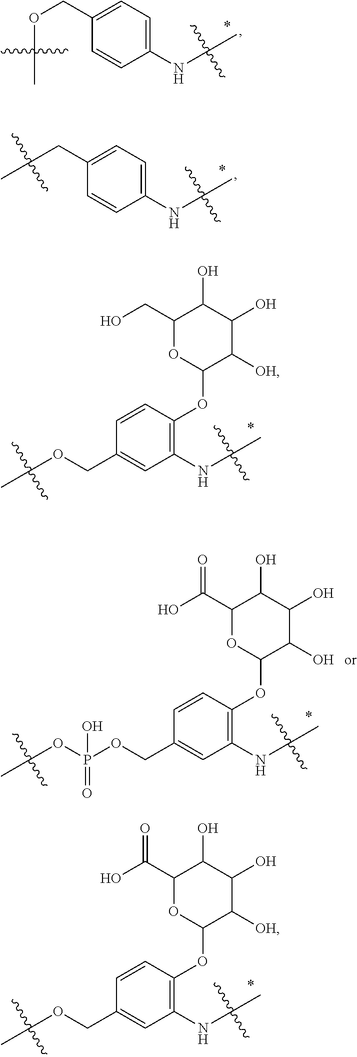 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

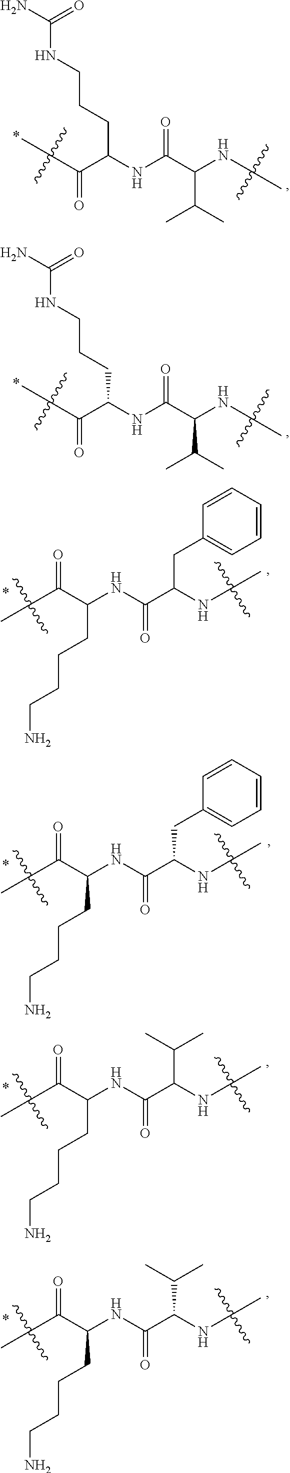

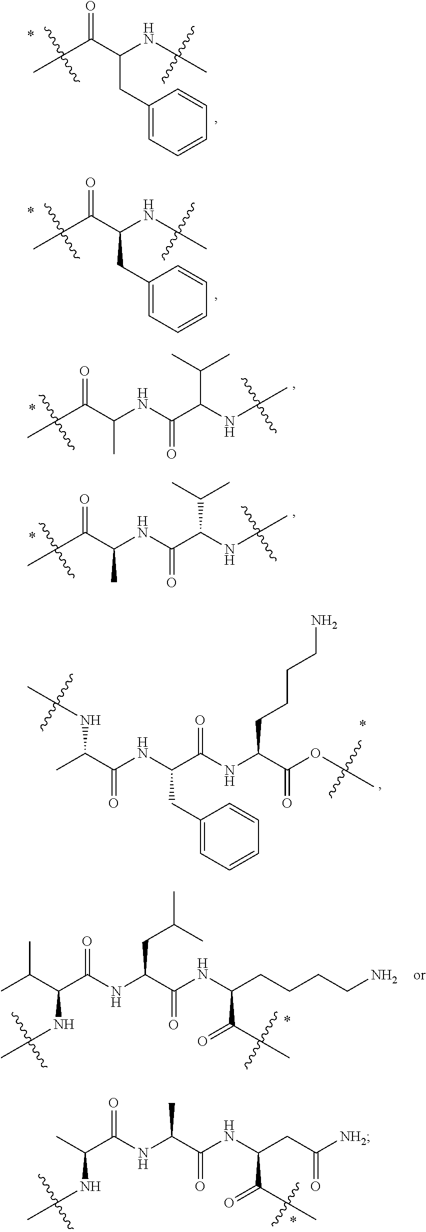

where the * of X₃ indicates the point of attachment to X₂;
each R⁷ is independently selected from H and C₁-C₆alkyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.
   In another aspect of the invention are RIG-1 agonists having the following structures:

a)
(SEQ ID NO: 334)
5′ ppp-GGACGUACGC (UUCG) GCGUACGUCC-3′
b)
(SEQ ID NO: 335)
5′ ppp-GGACGUACGC (UXCG) GCGUACGUCC-3′
c)
(SEQ ID NO: 336)
5′OH-GGACGUACGC (UUCG) GCGUACGUCC-3′
or
d)
(SEQ ID NO: 337)
5′OH-GGACGUACGC (UXCG) GCGUACGUCC-3′

where:
ppp-G is

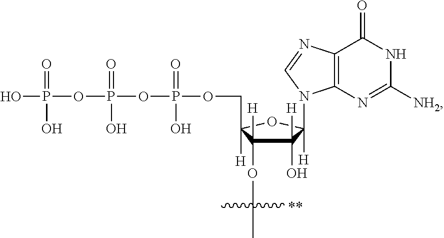

where the * of ppp-G is the point of attachment toward the 3′ end;
OH-G is

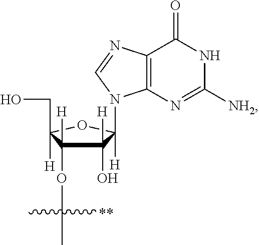

where the * of OH-G is the point of attachment toward the 3′ end;
G is

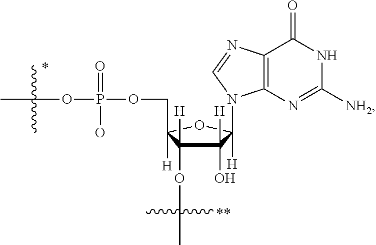

where the * of G is the point of attachment toward the 5′ end and the ** of G is the point of attachment toward the 3′ end;
A is

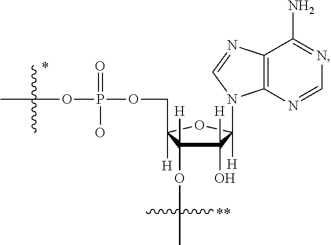

where the * of A is the point of attachment toward the 5′ end and the ** of A is the point of attachment the point of attachment toward the 3′ end;
C is

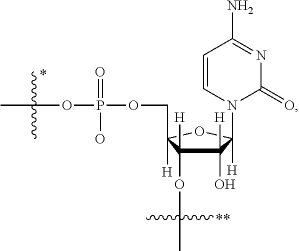

where the * of C is the point of attachment toward the 5′ end and the ** of C is the point of attachment toward the 3′ end;
or if C is in a 3′ terminal position, then C is

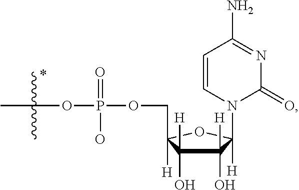

where the * of C is the point of attachment toward the 5′ end;
U is

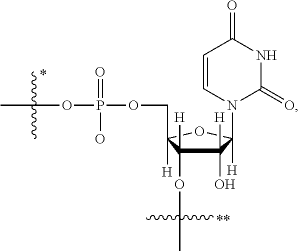

where the * of U is the point of attachment toward the 5′ end and the ** of U is the point of attachment toward the 3′ end;
and
X is

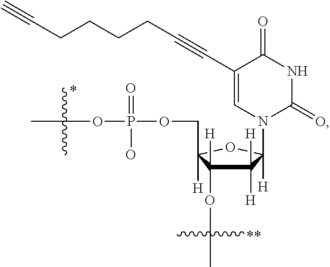

the * of X is the point of attachment toward the 5′ end and the ** of X is the point of attachment toward the 3′ end.

In another aspect of the invention are compounds having the structure of Formula (IV), and the pharmaceutically acceptable salts thereof, which are RIG-1 agonists:

RIGIa-L-R⁴ (IV)

wherein:
RIGIa is a RIG-1 agonist selected from:

a)
(SEQ ID NO: 338)
5′ ppp-GGACGUACGC (UX_{M}CG) GCGUACGUCC-3′
or
b)
(SEQ ID NO: 339)
5′OH-GGACGUACGC (UX_{M}CG) GCGUACGUCC-3′

where:
   ppp-G is

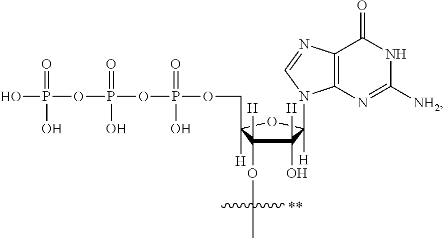

where the ** of ppp-G is the point of attachment toward the 3′ end;
   OH-G is

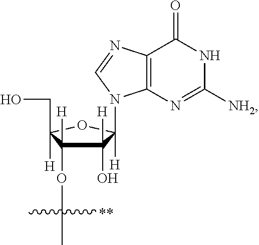

where the ** of OH-G is the point of attachment toward the 3′ end;
   G is

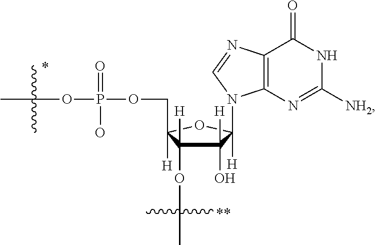

where the * of G is the point of attachment toward the 5′ end and the ** of G is the point of attachment toward the 3′ end;
   A is

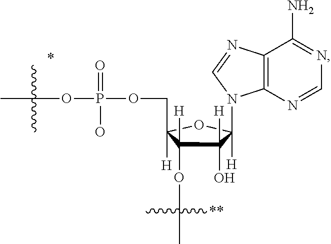

where the * of A is the point of attachment toward the 5′ end and the ** of A is the point of attachment the point of attachment toward the 3′ end;
   C is

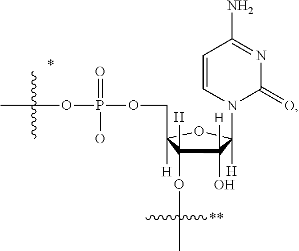

where the * of C is the point of attachment toward the 5′ end and the ** of C is the point of attachment toward the 3′ end;
   or if C is in a 3′ terminal position, then C is

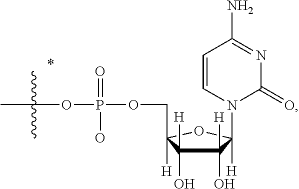

where the * of C is the point of attachment toward the 5′ end;
   U is

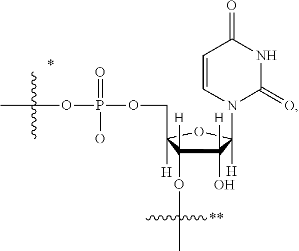

where the * of U is the point of attachment toward the 5′ end and the ** of U is the point of attachment toward the 3′ end;
   and
   X_{M} is

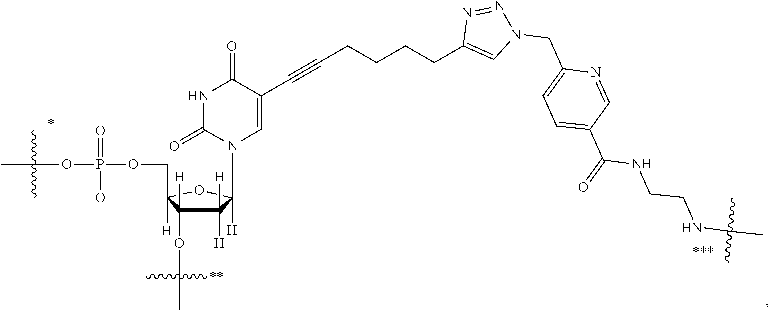

where the * of X_{M} is the point of attachment toward the 5′ end, the ** of X_{M} is the point of attachment toward the 3′ end and the *** of X_{M} is the point of attachment to L;
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—**; —C(═O)X₂X₃((CH₂)ₙO)ₜ(CH₂)ₙ—**, —C(═O)X₂X₃C(═O)(CH₂)ₘO(CH₂)ₘC(═O)—**; —(CH₂)ₙNHC(═O)(CH₂)ₙ—, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)O(CH₂)ₙC(R₇)₂SS(CH₂)ₙNHC(═O)(CH₂)ₙ—** or —C(═O)(CH₂)ₙC(═O)NH(CH₂)ₙ—**, where the ** of L indicates the point of attachment to R⁴;
R⁴ is

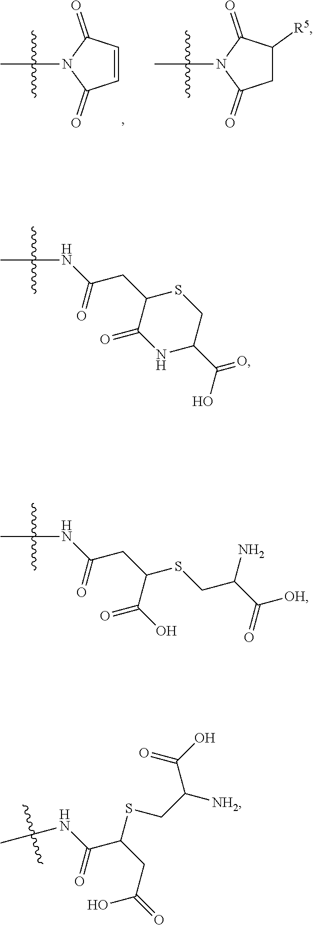

—ONH₂, —NH₂,

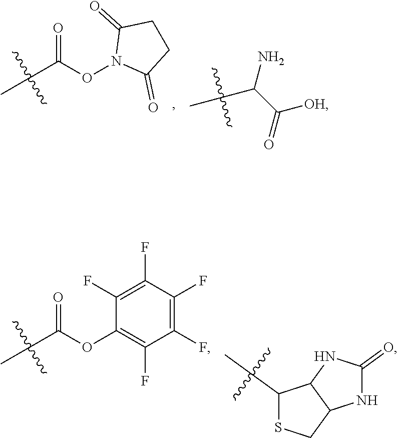 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

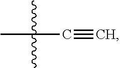 [SMILES: *C#C]

—NHC(═O)CH═CH₂, —SH, —SR, —OH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

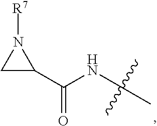

—CO₂H, —C(O)NHNH₂,

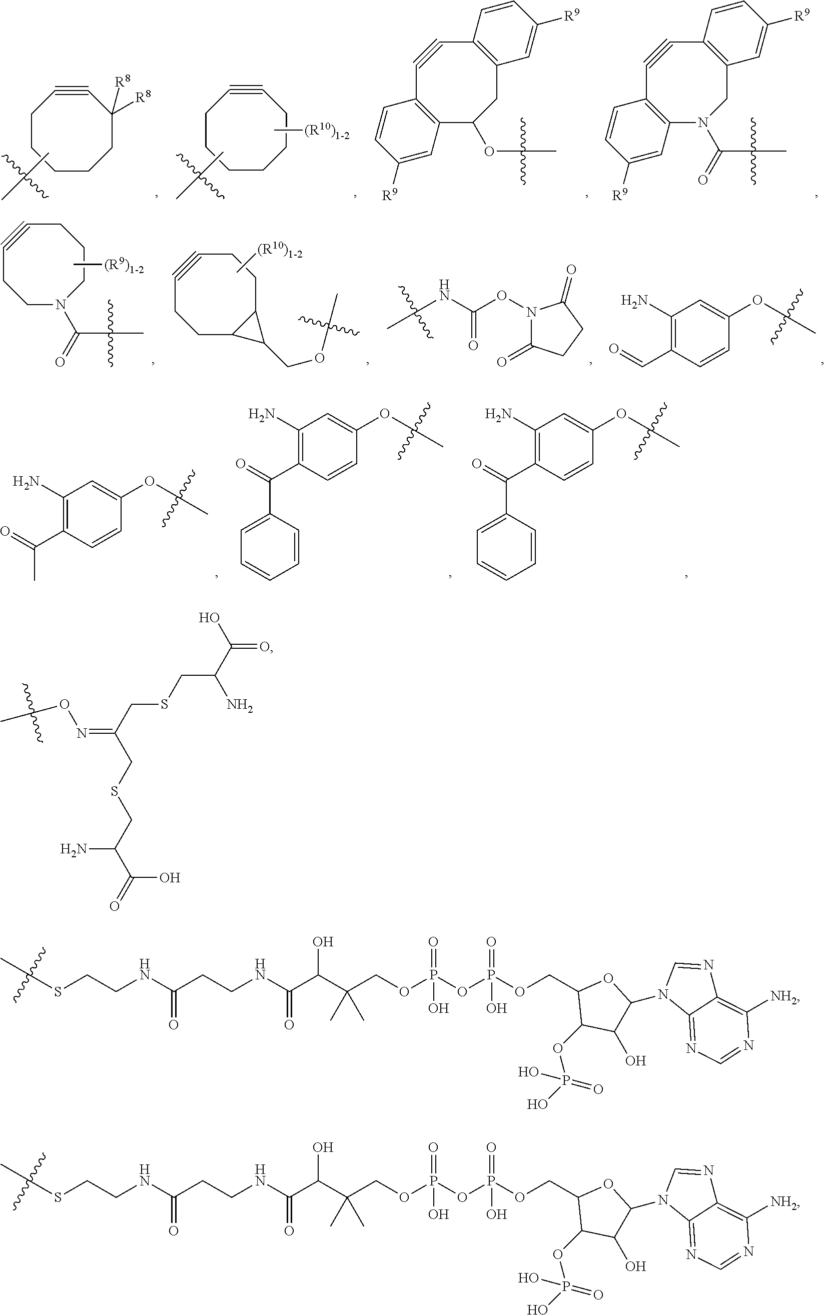

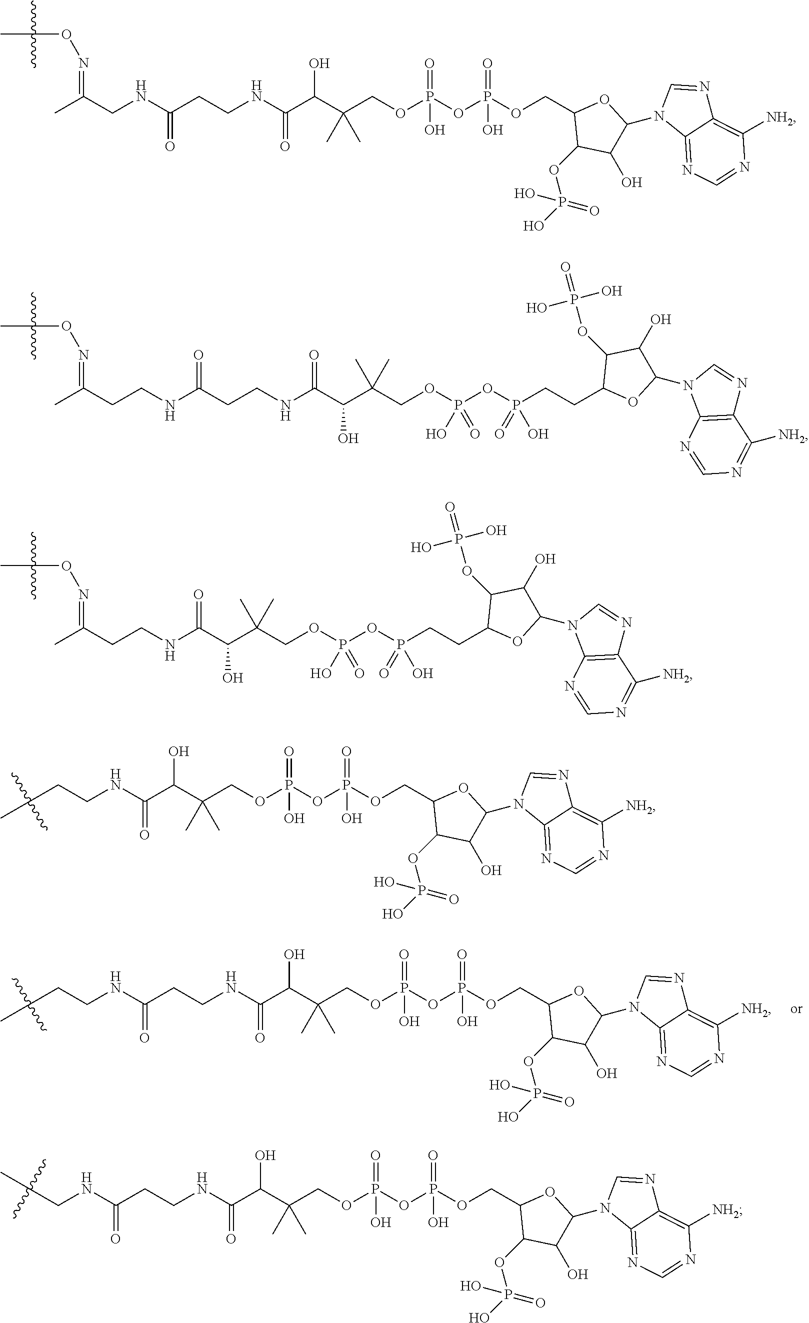

R⁵ is

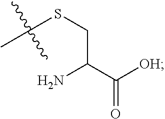 [SMILES: *SCC(N)C(=O)O]

X₁ is

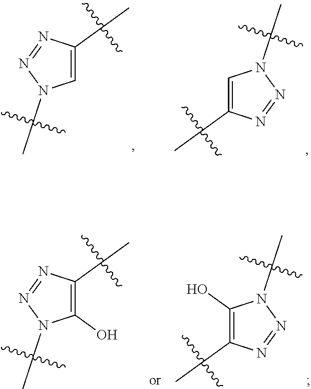 [SMILES: *c1cn(*)nn1 ; *c1cn(*)nn1 ; *c1nnn(*)c1O ; *c1nnn(*)c1O]

X₂ is

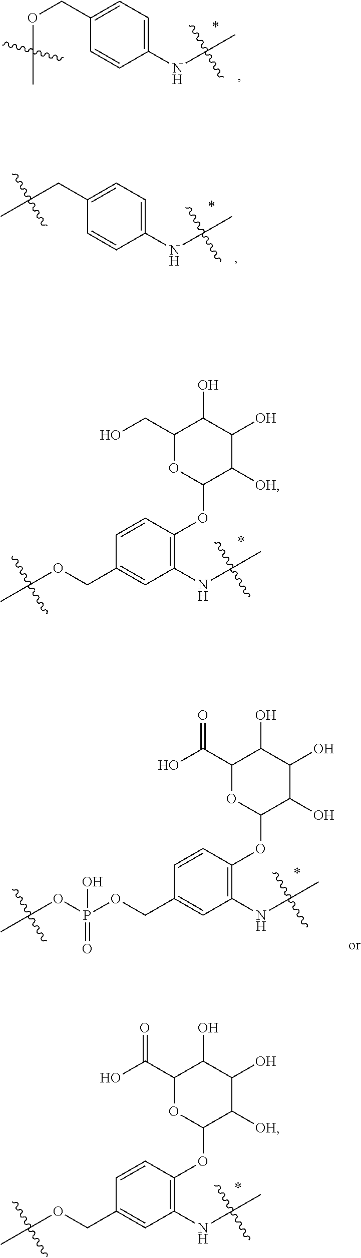 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

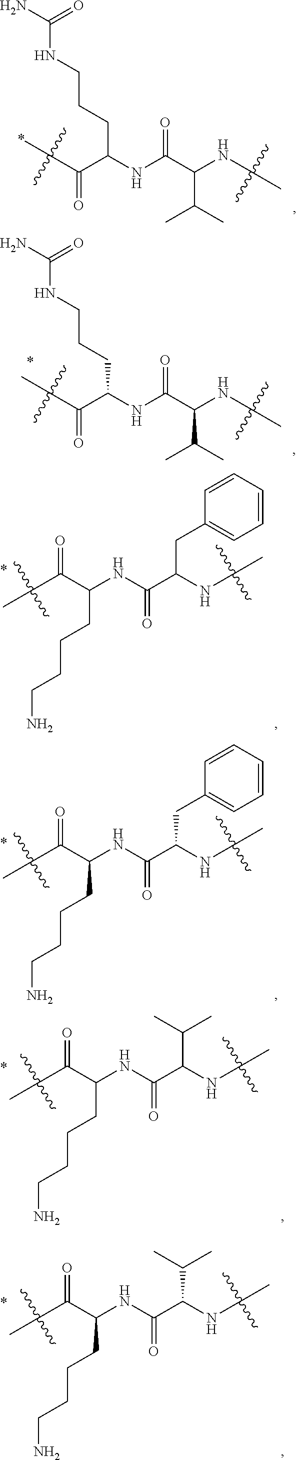

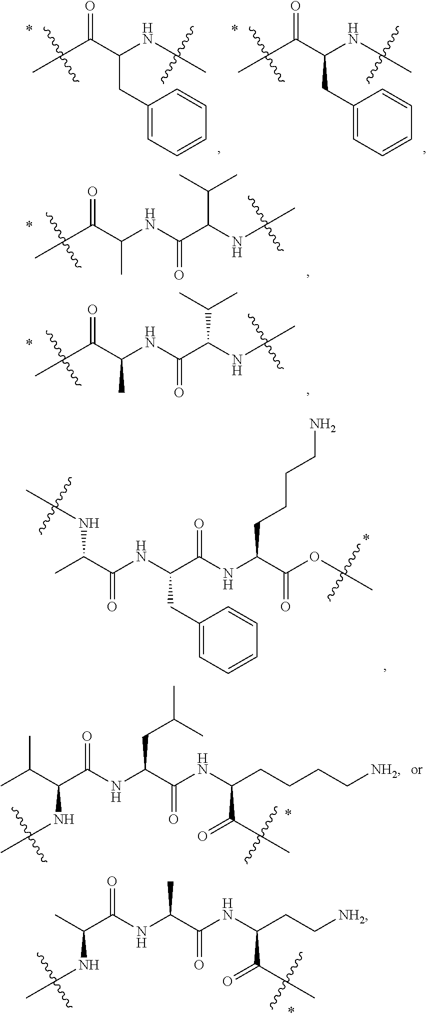

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

Another aspect of the invention are antibody conjugates having the structure of Formula (V), and the pharmaceutically acceptable salts thereof:

(RIGIa-L-R⁴⁰)y-Ab Formula (V)

wherein:
RIGIa is a RIG-1 agonist selected from:

a)
(SEQ ID NO: 338)
5′ ppp-GGACGUACGC (UX_{M}CG) GCGUACGUCC-3′
b)
(SEQ ID NO: 339)
5′OH-GGACGUACGC (UX_{M}CG) GCGUACGUCC-3′

where:
   ppp-G is

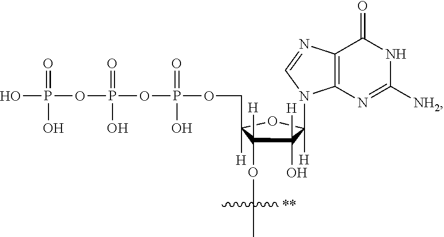

where the ** of ppp-G is the point of attachment toward the 3′ end;
   OH-G is

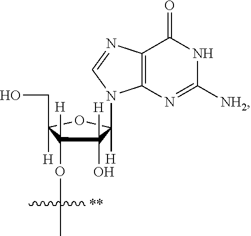

where the ** of OH-G is the point of attachment toward the 3′ end;
   G is

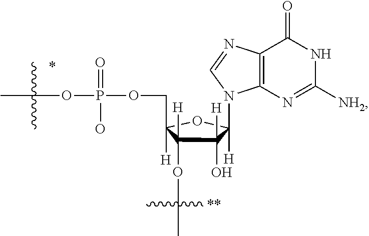

where the * of G is the point of attachment toward the 5′ end and the ** of G is the point of attachment toward the 3′ end;
   A is

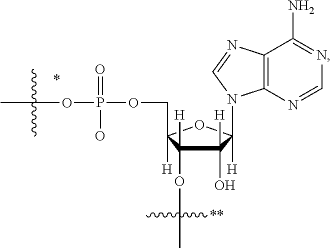

where the * of A is the point of attachment toward the 5′ end and the ** of A is the point of attachment the point of attachment toward the 3′ end;
   C is

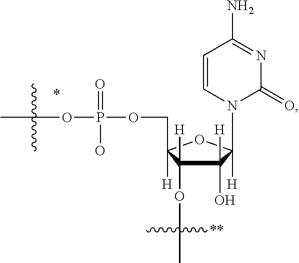

where the * of C is the point of attachment toward the 5′ end and the ** of C is the point of attachment toward the 3′ end;
   or if C is in a 3′ terminal position, then C is

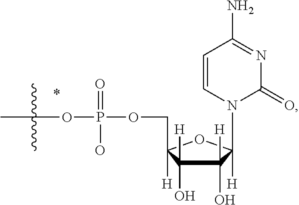

where the * of C is the point of attachment toward the 5′ end;
   U is

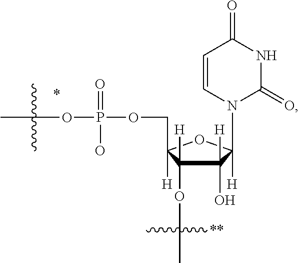

where the * of U is the point of attachment toward the 5′ end and the ** of U is the point of attachment toward the 3′ end;
   and
   X_{M} is

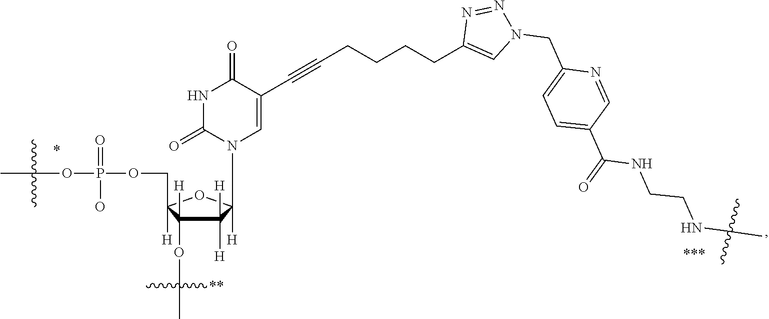

where the * of X_{M} is the point of attachment toward the 5′ end, the ** of X_{M} is the point of attachment toward the 3′ end and the *** of X_{M} is the point of attachment to L;
Ab is an antibody or antigen binding fragment thereof that specifically binds to human DC-SIGN;
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—**; —C(═O)X₂X₃((CH₂)ₙO)ₜ(CH₂)ₙ—**, —C(═O)X₂X₃C(═O)(CH₂)ₘO(CH₂)ₘC(═O)—**; —(CH₂)ₙNHC(═O)(CH₂)ₙ—, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)O(CH₂)ₙC(R₇)₂SS(CH₂)ₙNHC(═O)(CH₂)ₙ—** or —C(═O)(CH₂)ₙC(═O)NH(CH₂)ₙ—**, where the ** of L indicates the point of attachment to R⁴⁰;
R⁴⁰ is

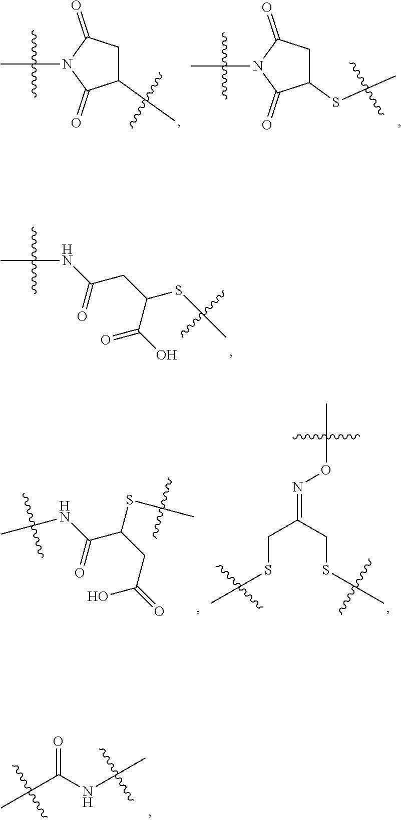 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—,

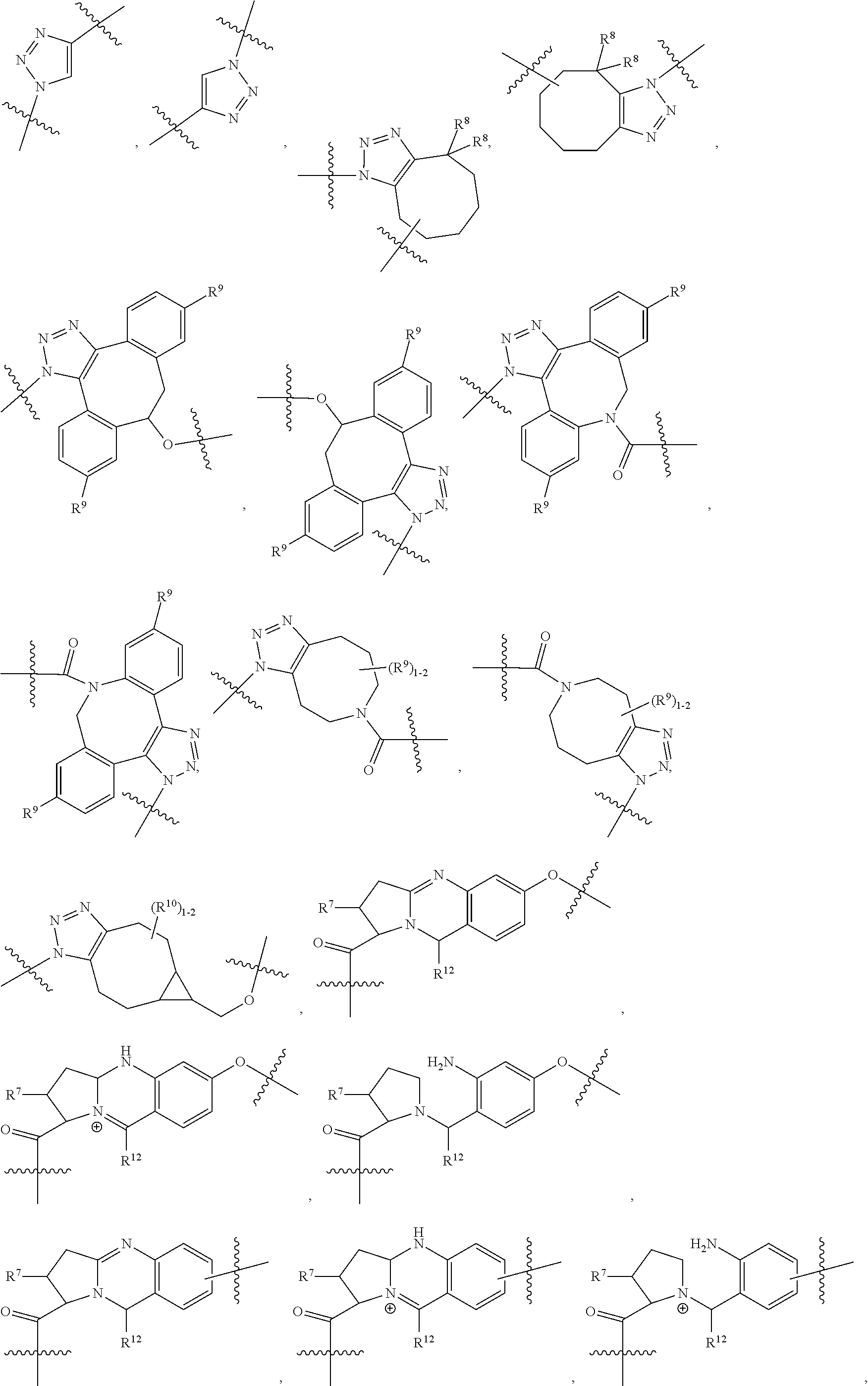

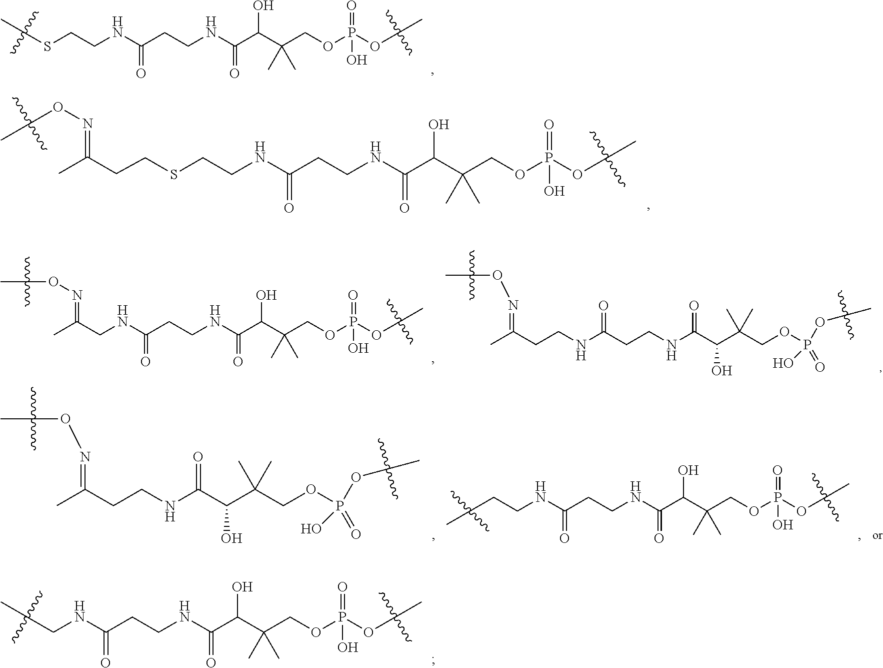

X₁ is

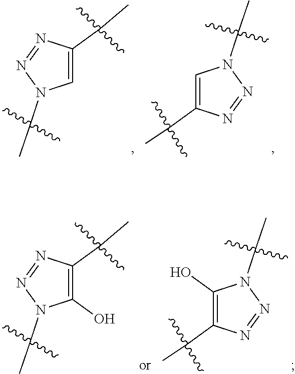 [SMILES: *c1cn(*)nn1 ; *c1cn(*)nn1 ; *c1nnn(*)c1O ; *c1nnn(*)c1O]

X₂ is

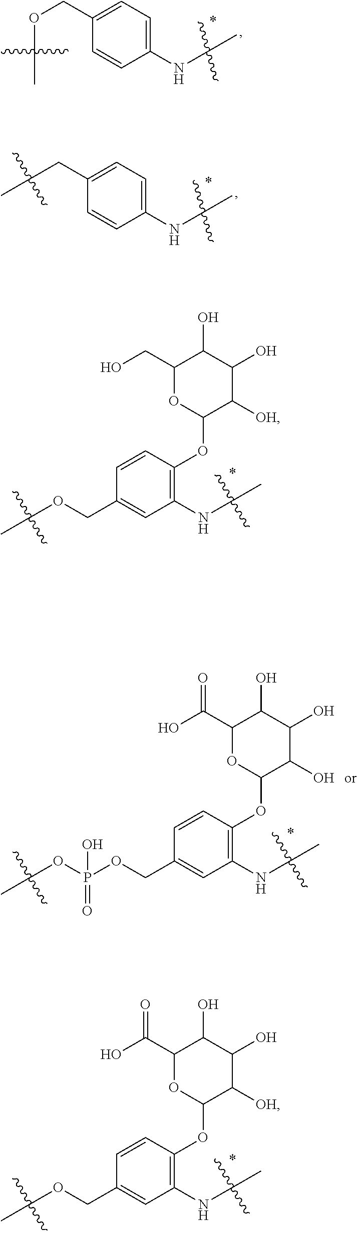 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

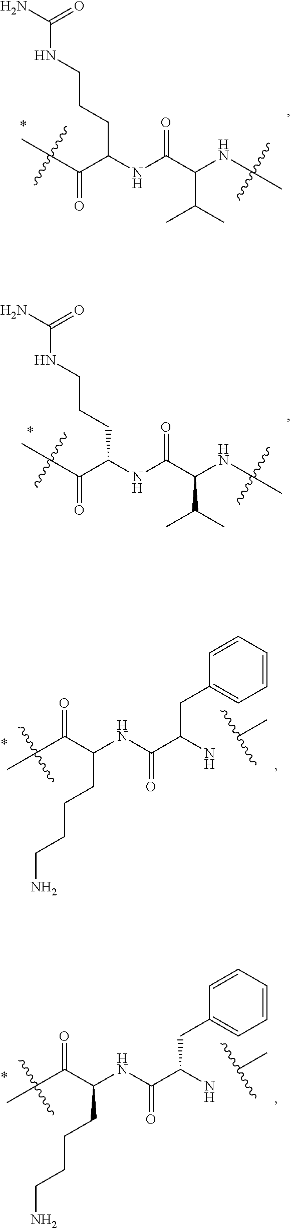

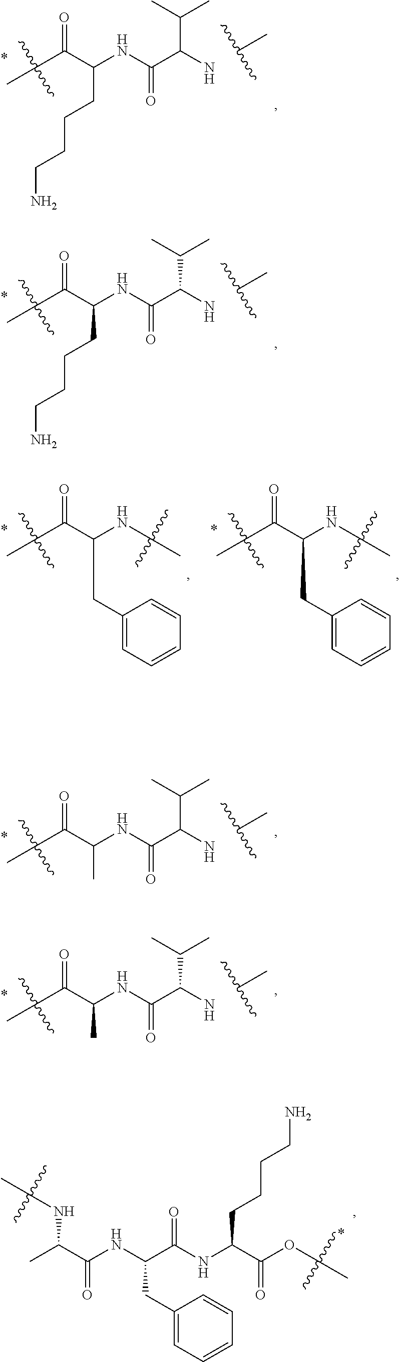

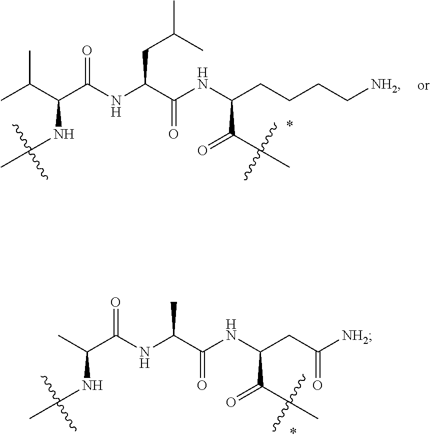 [SMILES: *N[C@H](C(=O)N[C@@H](CC(C)C)C(=O)N[C@@H](CCCCN)C(*)=O)C(C)C ; *N[C@@H](C)C(=O)N[C@@H](C)C(=O)N[C@@H](CC(N)=O)C(*)=O]

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Another aspect of the invention is a fusion protein comprising an anti-DC-SIGN antibody or antigen binding fragment thereof, or a DC-SIGN antibody conjugate disclosed herein linked to a peptide antigen. In some embodiments, the peptide antigen is linked directly or indirectly to the antibody or antigen binding fragment thereof. In some embodiments, the peptide antigen is linked to the N-terminus, C-terminus, or an internal site of the light chain or heavy chain of the antibody or antigen binding fragment thereof. In some embodiments, the peptide antigen is inserted into a CDR of the antibody or antigen binding fragment thereof.

Another aspect of the invention is a pharmaceutical composition that includes a therapeutically effective amount of an antibody or antigen binding fragment thereof, an antibody conjugate of Formula (II), Formula (IIa), Formula (IIb) or Formula (V), or a fusion protein disclosed herein, or a pharmaceutically acceptable salt thereof, and a pharmaceutically acceptable carrier.

Another aspect of the invention is a method for treating cancer, wherein the method comprises administering to a subject in need of such treatment an effective amount of an antibody or antigen binding fragment thereof, an antibody conjugate of Formula (II), Formula (IIa), Formula (IIb) or Formula (V), or a fusion protein disclosed herein, or pharmaceutically acceptable salt thereof. A cancer can be any of sarcomas, adenocarcinomas, blastomas, carcinomas, liver cancer, lung cancer, non-small cell lung cancer, small cell lung cancer, breast cancer, lymphoid cancer, colon cancer, renal cancer, urothelial cancer, prostate cancer, cancer of the pharynx, rectal cancer, renal cell carcinoma, cancer of the small intestine, esophageal cancer, melanoma, bone cancer, pancreatic cancer, skin cancer, cancer of the head or neck, cutaneous or intraocular malignant melanoma, uterine cancer, ovarian cancer, colorectal cancer, cancer of the anal region, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, environmentally induced cancers including those induced by asbestos, leukemia, lymphoma, acute myelogenous leukemia (AML), acute lymphoid leukemia (ALL), chronic myelogenous leukemia (CML), chronic lymphoid leukemia (CLL), myelodysplastic syndromes, B-cell acute lymphoid leukemia (“BALL”), T-cell acute lymphoid leukemia (“TALL”), B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia, myelodysplastic syndrome, plasmablastic lymphoma, plasmacytoid dendritic cell neoplasm, and Waldenstrom macroglobulinemia.

Another aspect of the invention is the use of an antibody or antigen binding fragment thereof, an antibody conjugate of Formula (II), Formula (IIa), Formula (IIb) or Formula (V), or a fusion protein disclosed herein, or pharmaceutically acceptable salt thereof, in the manufacture of a medicament for treating a cancer.

Another aspect of the invention is an antibody or antigen binding fragment thereof, an antibody conjugate, or a fusion protein disclosed herein for use in a method of medical treatment, wherein the method of medical treatment is for treating a cancer, and wherein the antibody conjugate is an antibody conjugate of Formula (II), Formula (IIa), Formula (IIb) or Formula (V), or pharmaceutically acceptable salt thereof. In addition, a further aspect of the invention is an antibody conjugate of Formula (II), Formula (IIa), Formula (IIb) or Formula (V), or a fusion protein disclosed herein for use in a method of suppressing a cancer for a sustained period and/or reducing recurrence of a cancer, when compared to an anti-DC-SIGN antibody alone.

Another aspect of the invention is a use of an antibody or antigen binding fragment thereof disclosed herein for detecting a cell expressing DC-SIGN. In some embodiments, the use is for monitoring or prognosing the onset, development, progression and/or severity of a disease or disorder as part of a clinical testing procedure, such as determining the efficacy of a particular therapy. In some embodiments, the antibody or antigen binding fragment thereof is conjugated to a detectable agent. In some embodiments, the detectable agent comprises various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, umbelliferone, fluorescein, fluorescein isothiocyanate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 64Cu, 113Sn, and 117Sn; and positron emitting metals using various positron emission tomographies, and non-radioactive paramagnetic metal ions. In some embodiments, the antibody, antibody fragment (e.g., antigen binding fragment) or functional equivalent is attached to solid supports, which include, but are not limited to, glass, cellulose, polyacrylamide, nylon, polystyrene, polyvinyl chloride or polypropylene.

Another aspect of the invention is a diagnostic reagent comprising an antibody or antigen binding fragment thereof disclosed herein. In some embodiments, the antibody or antigen binding fragment thereof is labeled with a radiolabel, a fluorophore, a chromophore, an imaging agent, or a metal ion.

Another aspect of the invention is a method of treating an autoimmune disease in a subject in need thereof, the method comprising administering to the subject a therapeutically effective amount of a fusion protein disclosed herein. In some embodiments, the autoimmune disease is selected from multiple sclerosis (myelin basic protein), insulin-dependent diabetes mellitus (glutamic acid decarboxylase), insulin-resistant diabetes mellitus (insulin receptor), coeliac disease (gliadin), bullous pemphigoid (collagen type XVII), auto-immune haemolytic anaemia (Rh protein), auto-immune thrombocytopenia (GpIIb/IIIa), myaesthenia gravis (acetylcholine receptor), Graves' disease (thyroid-stimulating hormone receptor), glomerulonephritis, such as Goodpasture's disease (alpha3(IV)NC1 collagen), pernicious anaemia (intrinsic factor), arthritis (e.g. rheumatoid arthritis), inflammatory bowel disease, gastritis, pernicious anaemia, thyroiditis, insulitis, diabetes, sialoadenitis, adrenalitis, autoimmune orchitis/oophoritis, glomerulonephritis, chronic obstructive pulmonary disease and experimental autoimmune encephalitis and multiple sclerosis.

### BRIEF DESCRIPTION OF THE DRAWINGS

FIGS. 1A-1D show exemplary data on DC-SIGN immunoconjugates activating human monocyte DCs and macrophages in vitro. 2B2 (DAPA)C-5 conjugate induced downregulation of DC-SIGN on monocyte dendritic cells and macrophages (FIGS. 1A and 1C), indicating target engagement. 2B2 (DAPA)C-5 conjugate induced monocyte dendritic cell and macrophage activation as measured by CD86 upregulation (FIGS. 1B and 1D).

FIGS. 2A-2D show exemplary data on DC-SIGN immunoconjugates activating human DCs and cytokine secretion in Tg+ mice. Tg+ mice treated with 2B2 (DAPA)C-5 had a significant downregulation of surface DC-SIGN (FIG. 2A), indicating target engagement, and a significant upregulation of CD86 on the surface of dendritic cells indicating activation (FIG. 2B). Plasma IL-12p70 (FIG. 2D) and IP-10 (FIG. 2C) were significantly increased in Tg+ mice treated with 2B2 (DAPA)C-5 at 6 hours post dose, indicative of on-target activation through DC-SIGN. * denotes a p value of <0.05, ** denotes a p value of <0.01 compared to Tg+ mice treated with isotype control (DAPA)C-5, calculated using an unpaired Student's t test.

FIGS. 3A-3B show exemplary data on DC-SIGN immunoconjugates activating DCs in an MC38 tumor model. Tg+ mice treated with 1 mg/kg of 2B2 (DAPA)C-5 had a significant upregulation of CD86 on the surface of DCs in the spleen (FIG. 3A) and CD11b+ CD11c+ MHCII+ cells in the tumor (FIG. 3B) (a mixed population consisting of dendritic cells, myeloid derived suppressor cells (MDSCs) and other antigen presenting cells). **** indicates p<0.001, * indicates p<0.05 using an unpaired Student's t test compared to saline treated Tg+ mice.

FIGS. 4A-4B show exemplary data on RIG-1 agonistic hairpins activating DC-SIGN expressing human DCs in vitro. C-70, C-71, C-72 and C-73 all induced monocyte dendritic cell activation as measured by CD86 upregulation (FIG. 4A) and interferon alpha secretion (FIG. 4B) by moDCs in a dose dependent manner.

### DETAILED DESCRIPTION OF THE INVENTION

Various enumerated embodiments of the invention are described herein. It will be recognized that features specified in each embodiment may be combined with other specified features to provide further embodiments of the present invention.

Throughout the text of this application, should there be a discrepancy between the text of the specification (e.g., Table 1) and the sequence listing, the text of the specification shall prevail.

### Definitions

The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl, ethyl, 1-methylethyl, n-propyl, isopropyl, n-butyl, isobutyl, sec-butyl, tert-butyl, n-pentyl, isopentyl and hexyl. Non-limiting examples of “C₃-C₆alkyl” groups include 1-methylethyl, n-propyl, isopropyl, n-butyl, isobutyl, sec-butyl, tert-butyl, n-pentyl, isopentyl and hexyl. Non-limiting examples of “C₄-C₆alkyl” groups include n-butyl, isobutyl, sec-butyl, tert-butyl, n-pentyl, isopentyl and hexyl.

As used herein, “DC-SIGN” (Dendritic Cell-Specific Intercellular adhesion molecule-3-Grabbing Non-integrin, also known as CD209; CD209 molecule, CDSIGN; CLEC4L; DC-SIGN1) refers to a transmembrane receptor and is referred to as DC-SIGN because of its expression on the surface of dendritic cells and macrophages. The protein is involved in the innate immune system and recognizes numerous evolutionarily divergent pathogens ranging from parasites to viruses with a large impact on public health. The protein is organized into three distinct domains: an N-terminal transmembrane domain, a tandem-repeat neck domain and C-type lectin carbohydrate recognition domain. The extracellular region consisting of the C-type lectin and neck domains has a dual function as a pathogen recognition receptor and a cell adhesion receptor by binding carbohydrate ligands on the surface of microbes and endogenous cells. The neck region is important for homo-oligomerization which allows the receptor to bind multivalent ligands with high avidity. Variations in the number of 23 amino acid repeats in the neck domain of this protein are rare but have a significant impact on ligand binding ability. Human DC-SIGN is encoded by the CD209 gene (GeneID 30835) which is closely related in terms of both sequence and function to a neighboring gene (GeneID 10332; often referred to as L-SIGN). DC-SIGN and L-SIGN differ in their ligand-binding properties and distribution. Alternative splicing results in multiple variants. The human CD209 gene is mapped to chromosomal location 19p13.2, and the genomic sequence of CD209 gene can be found in GenBank at NG_012167.1. In humans, there are seven DC-SIGN isoforms: 1, 3, 4, 5, 6, 7, and 8; the term “DC-SIGN” is used herein to refer collectively to all DC-SIGN isoforms. As used herein, a human DC-SIGN protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with DC-SIGN isoforms: 1, 3, 4, 5, 6, 7, and 8, wherein such proteins still have at least one of the functions of DC-SIGN. The mRNA and protein sequences for human DC-SIGN isoform 1, the longest isoform, are:

| Homo sapiens CD209 molecule (CD209), transcript variant 1, mRNA [NM_021155.3] | |
|---|---|
| (SEQ ID NO: 302) | |
| 1 | atcacagggt gggaaataaa agctgtggcc cccaggagtt ctggacactg ggggagagtg |
| 61 | gggtgacatg agtgactcca aggaaccaag actgcagcag ctgggcctcc tggaggagga |
| 121 | acagctgaga ggccttggat tccgacagac tcgaggatac aagagcttag cagggtgtct |
| 181 | tggccatggt cccctggtgc tgcaactcct ctccttcacg ctcttggctg ggctccttgt |
| 241 | ccaagtgtcc aaggtcccca gctccataag tcaggaacaa tccaggcaag acgcgatcta |
| 301 | ccagaacctg acccagctta aagctgcagt gggtgagctc tcagagaaat ccaagctgca |
| 361 | ggagatctac caggagctga cccagctgaa ggctgcagtg ggtgagcttc cagagaaatc |
| 421 | taagctgcag gagatctacc aggagctgac ccggctgaag gctgcagtgg gtgagcttcc |
| 481 | agagaaatct aagctgcagg agatctacca ggagctgacc tggctgaagg ctgcagtggg |
| 541 | tgagcttcca gagaaatcta agatgcagga gatctaccag gagctgactc ggctgaaggc |
| 601 | tgcagtgggt gagcttccag agaaatctaa gcagcaggag atctaccagg agctgacccg |
| 661 | gctgaaggct gcagtgggtg agcttccaga gaaatctaag cagcaggaga tctaccagga |
| 721 | gctgacccgg ctgaaggctg cagtgggtga gcttccagag aaatctaagc agcaggagat |
| 781 | ctaccaggag ctgacccagc tgaaggctgc agtggaacgc ctgtgccacc cctgtccctg |
| 841 | ggaatggaca ttcttccaag gaaactgtta cttcatgtct aactcccagc ggaactggca |
| 901 | cgactccatc accgcctgca aagaagtggg ggcccagctc gtcgtaatca aaagtgctga |
| 961 | ggagcagaac ttcctacagc tgcagtcttc cagaagtaac cgcttcacct ggatgggact |
| 1021 | ttcagatcta aatcaggaag gcacgtggca atgggtggac ggctcacctc tgttgcccag |
| 1081 | cttcaagcag tattggaaca gaggagagcc caacaacgtt ggggaggaag actgcgcgga |
| 1141 | atttagtggc aatggctgga acgacgacaa atgtaatctt gccaaattct ggatctgcaa |
| 1201 | aaagtccgca gcctcctgct ccagggatga agaacagttt ctttctccag cccctgccac |
| 1261 | cccaaacccc cctcctgcgt agcagaactt cacccccttt taagctacag ttccttctct |
| 1321 | ccatccttcg accttcacaa aatctctggg actgttcttt gtcagattct tcctccttta |
| 1381 | gaaggctggg tcccattctg tccttcttgt catgcctcca atttcccctg gtgtagagct |
| 1441 | tgtttttctg gcccatcctt ggagctttat gagtgagctg gtgtgggatg cctttggggg |
| 1501 | tggacttgtg ttccaagaat ccactctctc ttccttttgg agattaggat atttgggttg |
| 1561 | ccatgtgtag ctgctatgtc ccctggggcg ttatcttata catgcaaacc taccatctgt |
| 1621 | tcaacttcca cctaccacct cctgcacccc tttgatcggg gacttactgg ttgcaagagc |
| 1681 | tcattttgca ggctggaagc accagggaat taattccccc agtcaaccaa tggcacccag |
| 1741 | agagggcatg gaggctccac gcaacccctt ccacccccac atcttccttt gtcttataca |
| 1801 | tggcttccat ttggctgttt ctaagttgta ttctttattt tattattatt attactattt |
| 1861 | ttcgagatgg agtttcactc ttgtcgctca ggctggagtg ccatggcgcg atcttggctc |
| 1921 | actgcaacct ctgcctcccg ggttcaagtg attctcctgc ctcagcctca cgagtagctg |
| 1981 | gaattacagg caggcgccac cagacccggc taattttttg tatttttagt acagatgggg |
| 2041 | tttctccgtg ttggtcaggc tggtcttgaa ctcccgacct cagatgatct gcccgcctcg |
| 2101 | gcctcccaaa attgctggga ttacaggtgt gagccaccgc gcctggccta ttattttttg |
| 2161 | taagaataaa acaggtttat tgggatttgg gactctgaac agttctgtct ctactacctg |
| 2221 | atctcctcct accacgactt tgggatctag aggagctttg gctccggctg tgacggctcc |
| 2281 | ggccgttctc actgcggctg caccggcccc cgctgcggtc actatttctt cctctgctag |
| 2341 | gtgaattgtg cctctcctgg ctctttgaca tgtgctagtg agatttcttc cttttccttt |
| 2401 | cggattcccc atttcttttg taggaatggt ctggactagg gttctccttc cccgcagcct |
| 2461 | gtagtattca tcgtggtggc ccaccctctc tctccccttg gagctcttgc caaaggagga |
| 2521 | gacaagcaga ggtctctatt ggatttctca acacctgaag aaagttgcag tgttttcctc |
| 2581 | ttggacattg ttgtatttca aataaaccac aaatcatcat tttccaccga gccactgggc |
| 2641 | agaattcaca ctgaagctgt cgtcctgcgt acataccatc gtccgttaaa cagagaaaga |
| 2701 | gctgcttggc attcttcttc cgactggtac tgaacatata tacttgcccc tcaggtgagg |
| 2761 | ttccaagttg caactgacct tgaactgaat cactctcccc acgttatttt ttaattacta |
| 2821 | ttttttttta aagatggggt cttgctctgt cgccaggctg gagtgcagtg gcgcgatcta |
| 2881 | ggctcactgc aacttccgcc tcccgggttc aagcgattct cctgcctcag cctcccgagt |
| 2941 | agctgggact ccactaaaag tacaaaaatt agctgggcgt gcaccactgc gcccagctaa |
| 3001 | ttcttgtatt tttggtagag acggggtttc aacatgttga ccaggatggt ctcgatctct |
| 3061 | tgacctcgtg attcgcccgc cgcgtcctcc caaagtgctg ggattacagg cctgagccac |
| 3121 | cgcgcccagt ctctccccac gttcttgaac tcgggcagca catcctcaca gaaatctagg |
| 3181 | aactgttggt aggtttcttc ctcgctgtac tccaggcttg cttcggagtc atagtcatcc |
| 3241 | ctcctgcact gctcctttcc aaacactgta aacatgcttt taataagaag ggtaggactg |
| 3301 | gatgttggga aatcatgtga acatctatct ccaaatctgc aagctcctgt tttactgtag |
| 3361 | aagggacaat taactccatc cttctccatg actctgaaat ccaagggggg gttccgggtt |
| 3421 | ttgccatgtg gcgccatttt ccaactcatt ttcagcctga tccagcatct tctggacagc |
| 3481 | ttccggtttt tgtttcttct gtcgtttctg ttcctcctcc tctctctctt tcctctgctg |
| 3541 | ttcttcccat tgttccttta actttcgctc ttgttcttgc cgttttctag ccacctcttc |
| 3601 | cttttccttc tttattctga attcttcttg tgccttctgc tctctcagca accactcctc |
| 3661 | atgtaatctt tgcctctctc ttccccatag cttttctagt tgttgttttt caataaaagt |
| 3721 | gtcctcctct ttctgtgaga gtcctgagtc cctcagtgga gcaagttcct gctggcgttt |
| 3781 | ctttcgtttc tccttcttca gggcggccct gtactttttg tggcttggtt tctctggaaa |
| 3841 | tgtcaccttt tcgggcgcag ccatcttgcc ggcaccgccc cgcccctcta gttgtatcct |
| 3901 | ttataataaa ctggtaaaca ttgtaaccgc agattcagcc caatctggtt caactttgtg |
| 3961 | taataaaatg gcgagttgtt tttcagttgt cgtggacccc caggttgcaa gttacatacc |
| 4021 | ctgggcatgt ccagatgaac gaagcgtgca aatccacgtg gaacctaagt gctcagaccg |
| 4081 | aggaacaggg actgagttaa gaagtggaca ccacgtggca tgatccttga tccaatcaga |
| 4141 | ttgagccctg gcgtgatcca gtcagatcaa gcctcctgaa tcccctcatt acaagatcca |
| 4201 | atcatatcat gcctcactac cctctgtata taaaatctgc cccagcctcc aacttggaga |
| 4261 | gacagatttg ggccagactc ctgtgtcctt gcttggctgc cttgcaataa atttttctct |
| 4321 | ctacaaaa |
| CD209 antigen isoform 4 [Homo sapiens][NP_066978.1] | |
| (SEQ ID NO: 303) | |
| 1 | msdskeprlq qlglleeeql rglgfrqtrg ykslagclgh gplvlqllsf tllagllvqv |
| 61 | skvpssisqe qsrqdaiyqn ltqlkaavge lseksklqei yqeltqlkaa vgelpekskl |
| 121 | qeiyqeltrl kaavgelpek sklqeiyqel twlkaavgel pekskmqeiy qeltrlkaav |
| 181 | gelpekskqq eiyqeltrlk aavgelpeks kqqeiyqelt rlkaavgelp ekskqqeiyq |
| 241 | eltqlkaave rlchpcpwew tffqgncyfm snsqrnwhds itackevgaq lvviksaeeq |
| 301 | nflqlqssrs nrftwmglsd lngegtwqwv dgspllpsfk qywnrgepnn vgeedcaefs |
| 361 | gngwnddkcn lakfwickks aascsrdeeq flspapatpn pppa |

The mRNA and protein sequences of the other human DC-SIGN isoforms can be found in GeneBank with the following Accession Nos:

DC-SIGN isoform 3: NM_001144896.1 (mRNA)→NP_001138368.1 (protein);

DC-SIGN isoform 4: NM_001144897.1 (mRNA)→NP_001138369.1 (protein);

DC-SIGN isoform 5: NM_001144893.1 (mRNA)→NP_001138365.1 (protein);

DC-SIGN isoform 6: NM_001144894.1 (mRNA)→NP_001138366.1 (protein);

DC-SIGN isoform 7: NM_001144895.1 (mRNA)→NP_001138367.1 (protein);

DC-SIGN isoform 8: NM_001144899.1 (mRNA)→NP_001138371.1 (protein);

All the sequences above are hereby incorporated by reference.

As used herein, “L-SIGN” (liver/lymph node-specific intracellular adhesion molecules-3 grabbing non-integrin, also known as CLEC4M, CD299; LSIGN; CD209L; DCSIGNR; HP10347; DC-SIGN2; DC-SIGNR) refers to a transmembrane receptor and is referred to as L-SIGN because of its expression in the endothelial cells of the lymph nodes and liver. The protein is involved in the innate immune system and recognizes numerous evolutionarily divergent pathogens ranging from parasites to viruses, with a large impact on public health. The protein is organized into three distinct domains: an N-terminal transmembrane domain, a tandem-repeat neck domain and C-type lectin carbohydrate recognition domain. The extracellular region consisting of the C-type lectin and neck domains has a dual function as a pathogen recognition receptor and a cell adhesion receptor by binding carbohydrate ligands on the surface of microbes and endogenous cells. The neck region is important for homo-oligomerization which allows the receptor to bind multivalent ligands with high avidity. Variations in the number of 23 amino acid repeats in the neck domain of this protein are common and have a significant impact on ligand binding ability. This gene is closely related in terms of both sequence and function to a neighboring gene (GeneID 30835; often referred to as DC-SIGN or CD209). DC-SIGN and L-SIGN differ in their ligand-binding properties and distribution. Alternative splicing results in multiple variants. The human L-SIGN is encoded by the CLEC4M gene (GeneID 10332) which is mapped to chromosomal location 19p13.2, and the genomic sequence of CLEC4M gene can be found in GenBank at NG_029190.1. In human, there are nine L-SIGN isoforms: 1, 2, 3, 7, 8, 9, 10, 11, and 12; the term “L-SIGN” is used herein to refer collectively to all L-SIGN isoforms. As used herein, a human L-SIGN protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with L-SIGN isoforms: 1, 2, 3, 7, 8, 9, 10, 11, and 12, wherein such proteins still have at least one of the functions of L-SIGN. The mRNA and protein sequences for human L-SIGN isoform 1, the longest isoform, are:

| Homo sapiens C-type lectin domain family 4 member M (CLEC4M), transcript | |
|---|---|
| variant 1, mRNA [NM_014257.4] | |
| (SEQ ID NO: 304) | |
| 1 | acccagcttc ctgtttgtct tcctgagaga cagtagattt agaaagtgag gatcagaggg |
| 61 | tggaaaataa aagctgtggt ccccaggagt cctgaacatc tggggacagc gggaaaacat |
| 121 | gagtgactcc aaggaaccaa gggtgcagca gctgggcctc ctggaagaag atccaacaac |
| 181 | cagtggcatc agactttttc caagagactt tcaattccag cagatacatg gccacaagag |
| 241 | ctctacaggg tgtcttggcc atggcgccct ggtgctgcaa ctcctctcct tcatgctctt |
| 301 | ggctggggtc ctggtggcca tccttgtcca agtgtccaag gtccccagct ccctaagtca |
| 361 | ggaacaatcc gagcaagacg caatctacca gaacctgacc cagcttaaag ctgcagtggg |
| 421 | tgagctctca gagaaatcca agctgcagga gatctaccag gagctgaccc agctgaaggc |
| 481 | tgcagtgggt gagttgccag agaaatccaa gctgcaggag atctaccagg agctgacccg |
| 541 | gctgaaggct gcagtgggtg agttgccaga gaaatccaag ctgcaggaga tctaccagga |
| 601 | gctgacccgg ctgaaggctg cagtgggtga gttgccagag aaatccaagc tgcaggagat |
| 661 | ctaccaggag ctgacccggc tgaaggctgc agtgggtgag ttgccagaga aatccaagct |
| 721 | gcaggagatc taccaggagc tgacggagct gaaggctgca gtgggtgagt tgccagagaa |
| 781 | atccaagctg caggagatct accaggagct gacccagctg aaggctgcag tgggtgagtt |
| 841 | gccagaccag tccaagcagc agcaaatcta tcaagaactg accgatttga agactgcatt |
| 901 | tgaacgcctg tgccgccact gtcccaagga ctggacattc ttccaaggaa actgttactt |
| 961 | catgtctaac tcccagcgga actggcacga ctccgtcacc gcctgccagg aagtgagggc |
| 1021 | ccagctcgtc gtaatcaaaa ctgctgagga gcagaacttc ctacagctgc agacttccag |
| 1081 | gagtaaccgc ttctcctgga tgggactttc agacctaaat caggaaggca cgtggcaatg |
| 1141 | ggtggacggc tcacctctgt cacccagctt ccagcggtac tggaacagtg gagaacccaa |
| 1201 | caatagcggg aatgaagact gtgcggaatt tagtggcagt ggctggaacg acaatcgatg |
| 1261 | tgacgttgac aattactgga tctgcaaaaa gcccgcagcc tgcttcagag acgaatagtt |
| 1321 | gtttccctgc tagcctcagc ctccattgtg gtatagcaga acttcaccca cttgtaagcc |
| 1381 | agcgcttctt ctctccatcc ttggaccttc acaaatgccc tgagacggtt ctctgttcga |
| 1441 | tttttcatcc cctatgaacc tgggtcttat tctgtccttc tgatgcctcc aagtttccct |
| 1501 | ggtgtagagc ttgtgttctt ggcccatcct tggagcttta taagtgacct gagtgggatg |
| 1561 | catttagggg gcgggcttgg tatgttgtat gaatccactc tctgttcctt ttggagatta |
| 1621 | gactatttgg attcatgtgt agctgccctg tcccctgggg ctttatctca tccatgcaaa |
| 1681 | ctaccatctg ctcaacttcc agctacaccc cgtgcaccct tttgactggg gacttgctgg |
| 1741 | ttgaaggagc tcatcttgca ggctggaagc accagggaat taattccccc agtcaaccaa |
| 1801 | tggcatccag agagggcatg gaggctccat acaacctctt ccacccccac atctttcttt |
| 1861 | gtcctataca tgtcttccat ttggctgttt ctgagttgta gcctttataa taaagtggta |
| 1921 | aatgttgtaa ctgcaaaaaa aaaaaaaaaa aaaaaaaaaa aaaaaaaaaa aaaaaa |
| C-type lectin domain family 4 member M isoform 1 [Homo sapiens][NP_055072.3] | |
| (SEQ ID NO: 305) | |
| 1 | msdskeprvq qlglleedpt tsgirlfprd fqfqqihghk sstgclghga lvlqllsfml |
| 61 | lagvlvailv qvskvpssls qeqseqdaiy qnitqlkaav gelseksklq eiyqeltqlk |
| 121 | aavgelpeks klqeiyqelt rlkaavgelp eksklqeiyq eltrlkaavg elpeksklqe |
| 181 | iyqeltrlka avgelpeksk lqeiyqelte lkaavgelpe ksklqeiyqe ltqlkaavge |
| 241 | lpdqskqqqi yqeltdlkta ferlcrhcpk dwtffqgncy fmsnsqrnwh dsvtacqevr |
| 301 | aqlwiktae eqnflqlqts rsnrfswmgl sdlngegtwq wydgsplsps fqrywnsgep |
| 361 | nnsgnedcae fsgsgwndnr cdvdnywick kpaacfrde |

The mRNA and protein sequences of the other human L-SIGN isoforms can be found in GeneBank with the following Accession Nos:

L-SIGN isoform 2: NM_001144904.1 (mRNA)→NP_001138376.1 (protein);

L-SIGN isoform 3: NP_001138382.1 (mRNA)→NP_001138383.1 (protein);

L-SIGN isoform 7: NM_001144906.1 (mRNA)→NP_001138378.1 (protein);

L-SIGN isoform 8: NM_001144910.1 (mRNA)→NP_001138382.1 (protein);

L-SIGN isoform 9: NM_001144909.1 (mRNA)→NP_001138381.1 (protein);

L-SIGN isoform 10: NM_001144908.1 (mRNA)→NP_001138380.1 (protein);

L-SIGN isoform 11: NM_001144907.1 (mRNA)→NP_001138379.1 (protein);

L-SIGN isoform 12: NM_001144905.1 (mRNA)→NP_001138377.1 (protein);

All the sequences above are hereby incorporated by reference.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

The term “antibody fragment” or “antigen-binding fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. In a combined Kabat and Chothia numbering scheme for a given CDR region (for example, HC CDR1, HC CDR2, HC CDR3, LC CDR1, LC CDR2 or LC CDR3), in some embodiments, the CDRs correspond to the amino acid residues that are defined as part of the Kabat CDR, together with the amino acid residues that are defined as part of the Chothia CDR.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” Conformational and linear epitopes are distinguished in that the binding to the former but not the latter is lost in the presence of denaturing solvents.

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within an antibody can be replaced with other amino acid residues from the same side chain family and the altered antibody can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The terms “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric cancer including gastrointestinal cancer, pancreatic cancer, glioblastoma, neuroblastoma, cervical cancer, ovarian cancer, liver cancer, bladder cancer, urinary tract cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer.

The terms “combination” or “pharmaceutical combination,” as used herein mean a product that results from the mixing or combining of more than one active ingredient and includes both fixed and non-fixed combinations of the active ingredients. The term “fixed combination” means that the active ingredients, by way of example, a compound of the invention and one or more additional therapeutic agent, are administered to a subject simultaneously in the form of a single entity or dosage. The term “non-fixed combination” means that the active ingredients, by way of example, a compound of the invention and one or more additional therapeutic agent, are administered to a subject as separate entities either simultaneously, concurrently or sequentially with no specific time limits, wherein such administration provides therapeutically effective levels of the active ingredients in the body of the subject. The latter also applies to cocktail therapy, e.g. the administration of 3 or more active ingredients.

The terms “composition” or “pharmaceutical composition,” as used herein, refers to a mixture of a compound of the invention with at least one and optionally more than one other pharmaceutically acceptable chemical components, such as carriers, stabilizers, diluents, dispersing agents, suspending agents, thickening agents, and/or excipients.

The term “an optical isomer” or “a stereoisomer”, as used herein, refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non- superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold- Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “pharmaceutically acceptable salt,” as used herein, refers to a salt which does not abrogate the biological activity and properties of the compounds of the invention, and does not cause significant irritation to a subject to which it is administered.

The term “subject”, as used herein, encompasses mammals and non-mammals. Examples of mammals include, but are not limited to, humans, chimpanzees, apes, monkeys, cattle, horses, sheep, goats, swine; rabbits, dogs, cats, rats, mice, guinea pigs, and the like. Examples of non-mammals include, but are not limited to, birds, fish and the like. Frequently the subject is a human.

The term “a subject in need of such treatment”, refers to a subject which would benefit biologically, medically or in quality of life from such treatment.

The term “therapeutically effective amount,” as used herein, refers to an amount of an antibody conjugate of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of an antibody conjugate of the invention that, when administered to a subject, is effective to at least partially alleviate, inhibit, prevent and/or ameliorate a condition, or a disorder or a disease.

The term “STING” refers to STimulator of INterferon Genes receptor, also known as TMEM173, ERIS, MITA, MPYS, SAVI, or NET23. As used herein, the terms “STING” and “STING receptor” are used interchangeably, and include different isoforms and variants of STING. The mRNA and protein sequences for human STING isoform 1, the longest isoform, are:

| Homo sapiens transmembrane protein 173 (TMEM173), transcript variant 1, | |
|---|---|
| mRNA [NM_198282.3] | |
| [SEQ ID NO: 324] | |
| 1 | tataaaaata gctcttgtta ccggaaataa ctgttcattt ttcactcctc cctcctaggt |
| 61 | cacacttttc agaaaaagaa tctgcatcct ggaaaccaga agaaaaatat gagacgggga |
| 121 | atcatcgtgt gatgtgtgtg ctgcctttgg ctgagtgtgt ggagtcctgc tcaggtgtta |
| 181 | ggtacagtgt gtttgatcgt ggtggcttga ggggaacccg ctgttcagag ctgtgactgc |
| 241 | ggctgcactc agagaagctg cccttggctg ctcgtagcgc cgggccttct ctcctcgtca |
| 301 | tcatccagag cagccagtgt ccgggaggca gaagatgccc cactccagcc tgcatccatc |
| 361 | catcccgtgt cccaggggtc acggggccca gaaggcagcc ttggttctgc tgagtgcctg |
| 421 | cctggtgacc ctttgggggc taggagagcc accagagcac actctccggt acctggtgct |
| 481 | ccacctagcc tccctgcagc tgggactgct gttaaacggg gtctgcagcc tggctgagga |
| 541 | gctgcgccac atccactcca ggtaccgggg cagctactgg aggactgtgc gggcctgcct |
| 601 | gggctgcccc ctccgccgtg gggccctgtt gctgctgtcc atctatttct actactccct |
| 661 | cccaaatgcg gtcggcccgc ccttcacttg gatgcttgcc ctcctgggcc tctcgcaggc |
| 721 | actgaacatc ctcctgggcc tcaagggcct ggccccagct gagatctctg cagtgtgtga |
| 781 | aaaagggaat ttcaacgtgg cccatgggct ggcatggtca tattacatcg gatatctgcg |
| 841 | gctgatcctg ccagagctcc aggcccggat tcgaacttac aatcagcatt acaacaacct |
| 901 | gctacggggt gcagtgagcc agcggctgta tattctcctc ccattggact gtggggtgcc |
| 961 | tgataacctg agtatggctg accccaacat tcgcttcctg gataaactgc cccagcagac |
| 1021 | cggtgaccat gctggcatca aggatcgggt ttacagcaac agcatctatg agcttctgga |
| 1081 | gaacgggcag cgggcgggca cctgtgtcct ggagtacgcc acccccttgc agactttgtt |
| 1141 | tgccatgtca caatacagtc aagctggctt tagccgggag gataggcttg agcaggccaa |
| 1201 | actcttctgc cggacacttg aggacatcct ggcagatgcc cctgagtctc agaacaactg |
| 1261 | ccgcctcatt gcctaccagg aacctgcaga tgacagcagc ttctcgctgt cccaggaggt |
| 1321 | tctccggcac ctgcggcagg aggaaaagga agaggttact gtgggcagct tgaagacctc |
| 1381 | agcggtgccc agtacctcca cgatgtccca agagcctgag ctcctcatca gtggaatgga |
| 1441 | aaagcccctc cctctccgca cggatttctc ttgagaccca gggtcaccag gccagagcct |
| 1501 | ccagtggtct ccaagcctct ggactggggg ctctcttcag tggctgaatg tccagcagag |
| 1561 | ctatttcctt ccacaggggg ccttgcaggg aagggtccag gacttgacat cttaagatgc |
| 1621 | gtcttgtccc cttgggccag tcatttcccc tctctgagcc tcggtgtctt caacctgtga |
| 1681 | aatgggatca taatcactgc cttacctccc tcacggttgt tgtgaggact gagtgtgtgg |
| 1741 | aagtttttca taaactttgg atgctagtgt acttaggggg tgtgccaggt gtctttcatg |
| 1801 | gggccttcca gacccactcc ccacccttct ccccttcctt tgcccgggga cgccgaactc |
| 1861 | tctcaatggt atcaacaggc tccttcgccc tctggctcct ggtcatgttc cattattggg |
| 1921 | gagccccagc agaagaatgg agaggaggag gaggctgagt ttggggtatt gaatcccccg |
| 1981 | gctcccaccc tgcagcatca aggttgctat ggactctcct gccgggcaac tcttgcgtaa |
| 2041 | tcatgactat ctctaggatt ctggcaccac ttccttccct ggccccttaa gcctagctgt |
| 2101 | gtatcggcac ccccacccca ctagagtact ccctctcact tgcggtttcc ttatactcca |
| 2161 | cccctttctc aacggtcctt ttttaaagca catctcagat tacccaaaaa aaaaaaaaaa |
| 2221 | aaa |

Homo sapiens stimulator of interferon genes protein isoform 1 [NP_938023.1]
[SEQ ID NO: 325]
MPHSSLHPSIPCPRGHGAQKAALVLLSACLVTLWGLGEPPEHTLRYLVLHLASLQLGLLLNGVC
SLAEELRHIHSRYRGSYWRTVRACLGCPLRRGALLLLSIYFYYSLPNAVGPPFTWMLALLGLSQ
ALNILLGLKGLAPAEISAVCEKGNFNVAHGLAWSYYIGYLRLILPELQARIRTYNQHYNNLLRGAV
SQRLYILLPLDCGVPDNLSMADPNIRFLDKLPQQTGDHAGIKDRVYSNSIYELLENGQRAGTCV
LEYATPLQTLFAMSQYSQAGFSREDRLEQAKLFCRTLEDILADAPESQNNCRLIAYQEPADDSS
FSLSQEVLRHLRQEEKEEVTVGSLKTSAVPSTSTMSQEPELLISGMEKPLPLRTDFS

The mRNA and protein sequences for human STING isoform 2, a shorter isoform are:

| Homo sapiens transmembrane protein 173 (TMEM173), transcript variant 2, | |
|---|---|
| mRNA [NM_001301738.1] | |
| [SEQ ID NO: 326] | |
| 1 | gctgcactca gagaagctgc ccttggctgc tcgtagcgcc gggccttctc tcctcgtcat |
| 61 | catccagagc agccagtgtc cgggaggcag aagatgcccc actccagcct gcatccatcc |
| 121 | atcccgtgtc ccaggggtca cggggcccag aaggcagcct tggttctgct gagtgcctgc |
| 181 | ctggtgaccc tttgggggct aggagagcca ccagagcaca ctctccggta cctggtgctc |
| 241 | cacctagcct ccctgcagct gggactgctg ttaaacgggg tctgcagcct ggctgaggag |
| 301 | ctgcgccaca tccactccag gtaccggggc agctactgga ggactgtgcg ggcctgcctg |
| 361 | ggctgccccc tccgccgtgg ggccctgttg ctgctgtcca tctatttcta ctactccctc |
| 421 | ccaaatgcgg tcggcccgcc cttcacttgg atgcttgccc tcctgggcct ctcgcaggca |
| 481 | ctgaacatcc tcctgggcct caagggcctg gccccagctg agatctctgc agtgtgtgaa |
| 541 | aaagggaatt tcaacgtggc ccatgggctg gcatggtcat attacatcgg atatctgcgg |
| 601 | ctgatcctgc cagagctcca ggcccggatt cgaacttaca atcagcatta caacaacctg |
| 661 | ctacggggtg cagtgagcca gcggctgtat attctcctcc cattggactg tggggtgcct |
| 721 | gataacctga gtatggctga ccccaacatt cgcttcctgg ataaactgcc ccagcagacc |
| 781 | ggtgaccatg ctggcatcaa ggatcgggtt tacagcaaca gcatctatga gcttctggag |
| 841 | aacgggcagc ggaacctgca gatgacagca gcttctcgct gtcccaggag gttctccggc |
| 901 | acctgcggca ggaggaaaag gaagaggtta ctgtgggcag cttgaagacc tcagcggtgc |
| 961 | ccagtacctc cacgatgtcc caagagcctg agctcctcat cagtggaatg gaaaagcccc |
| 1021 | tccctctccg cacggatttc tcttgagacc cagggtcacc aggccagagc ctccagtggt |
| 1081 | ctccaagcct ctggactggg ggctctcttc agtggctgaa tgtccagcag agctatttcc |
| 1141 | ttccacaggg ggccttgcag ggaagggtcc aggacttgac atcttaagat gcgtcttgtc |
| 1201 | cccttgggcc agtcatttcc cctctctgag cctcggtgtc ttcaacctgt gaaatgggat |
| 1261 | cataatcact gccttacctc cctcacggtt gttgtgagga ctgagtgtgt ggaagttttt |
| 1321 | cataaacttt ggatgctagt gtacttaggg ggtgtgccag gtgtctttca tggggccttc |
| 1381 | cagacccact ccccaccctt ctccccttcc tttgcccggg gacgccgaac tctctcaatg |
| 1441 | gtatcaacag gctccttcgc cctctggctc ctggtcatgt tccattattg gggagcccca |
| 1501 | gcagaagaat ggagaggagg aggaggctga gtttggggta ttgaatcccc cggctcccac |
| 1561 | cctgcagcat caaggttgct atggactctc ctgccgggca actcttgcgt aatcatgact |
| 1621 | atctctagga ttctggcacc acttccttcc ctggcccctt aagcctagct gtgtatcggc |
| 1681 | acccccaccc cactagagta ctccctctca cttgcggttt ccttatactc cacccctttc |
| 1741 | tcaacggtcc ttttttaaag cacatctcag attacccaaa aaaaaaaaaa aaaaa |

Homo sapiens stimulator of interferon genes protein isoform 2
[NP_001288667.1]
[SEQ ID NO: 327]
MPHSSLHPSIPCPRGHGAQKAALVLLSACLVTLWGLGEPPEHTLRYLVLHLASLQLGLLLNGVC
SLAEELRHIHSRYRGSYWRTVRACLGCPLRRGALLLLSIYFYYSLPNAVGPPFTWMLALLGLSQ
ALNILLGLKGLAPAEISAVCEKGNFNVAHGLAWSYYIGYLRLILPELQARIRTYNQHYNNLLRGAV
SQRLYILLPLDCGVPDNLSMADPNIRFLDKLPQQTGDHAGIKDRVYSNSIYELLENGQRNLQMT
AASRCPRRFSGTCGRRKRKRLLWAA

The sequences of other human STING isoforms/SNPs (single nucleotide polymorphisms) include the following and those described in Yi, PLoS One. 2013 Oct. 21; 8(10):e77846.

hSTING wt (wild type): Reference SNP (refSNP) Cluster Report: rs1131769
[SEQ ID NO: 328]
atgccccactccagcctgcatccatccatcccgtgtcccaggggtcacggggcccagaaggcagccttggttctgctgagtgcctgcc
tggtgaccctttgggggctaggagagccaccagagcacactctccggtacctggtgctccacctagcctccctgcagctgggactgct
gttaaacggggtctgcagcctggctgaggagctgcgccacatccactccaggtaccggggcagctactggaggactgtgcgggcct
gcctgggctgccccctccgccgtggggccctgttgctgctgtccatctatttctactactccctcccaaatgcggtcggcccgcccttcact
tggatgcttgccctcctgggcctctcgcaggcactgaacatcctcctgggcctcaagggcctggccccagctgagatctctgcagtgtgt
gaaaaagggaatttcaacgtggcccatgggctggcatggtcatattacatcggatatctgcggctgatcctgccagagctccaggccc
ggattcgaacttacaatcagcattacaacaacctgctacggggtgcagtgagccagcggctgtatattctcctcccattggactgtggg
gtgcctgataacctgagtatggctgaccccaacattcgcttcctggataaactgccccagcagaccggtgaccgtgctggcatcaagg
atcgggtttacagcaacagcatctatgagcttctggagaacgggcagcgggcgggcacctgtgtcctggagtacgccacccccttgc
agactttgtttgccatgtcacaatacagtcaagctggctttagccgggaggataggcttgagcaggccaaactcttctgccggacacttg
aggacatcctggcagatgcccctgagtctcagaacaactgccgcctcattgcctaccaggaacctgcagatgacagcagcttctcgct
gtcccaggaggttctccggcacctgcggcaggaggaaaaggaagaggttactgtgggcagcttgaagacctcagcggtgcccagt
acctccacgatgtcccaagagcctgagctcctcatcagtggaatggaaaagcccctccctctccgcacggatttctcttga
hSTING R293Q: Reference SNP (refSNP) Cluster Report: rs1131769 rs7380824
[SEQ ID NO: 329]
atgccccactccagcctgcatccatccatcccgtgtcccaggggtcacggggcccagaaggcagccttggttctgctgagtgcctgcc
tggtgaccctttgggggctaggagagccaccagagcacactctccggtacctggtgctccacctagcctccctgcagctgggactgct
gttaaacggggtctgcagcctggctgaggagctgcgccacatccactccaggtaccggggcagctactggaggactgtgcgggcct
gcctgggctgccccctccgccgtggggccctgttgctgctgtccatctatttctactactccctcccaaatgcggtcggcccgcccttcact
tggatgcttgccctcctgggcctctcgcaggcactgaacatcctcctgggcctcaagggcctggccccagctgagatctctgcagtgtgt
gaaaaagggaatttcaacgtggcccatgggctggcatggtcatattacatcggatatctgcggctgatcctgccagagctccaggccc
ggattcgaacttacaatcagcattacaacaacctgctacggggtgcagtgagccagcggctgtatattctcctcccattggactgtggg
gtgcctgataacctgagtatggctgaccccaacattcgcttcctggataaactgccccagcagaccggtgaccgtgctggcatcaagg
atcgggtttacagcaacagcatctatgagcttctggagaacgggcagcgggcgggcacctgtgtcctggagtacgccacccccttgc
agactttgtttgccatgtcacaatacagtcaagctggctttagccgggaggataggcttgagcaggccaaactcttctgccagacacttg
aggacatcctggcagatgcccctgagtctcagaacaactgccgcctcattgcctaccaggaacctgcagatgacagcagcttctcgct
gtcccaggaggttctccggcacctgcggcaggaggaaaaggaagaggttactgtgggcagcttgaagacctcagcggtgcccagt
acctccacgatgtcccaagagcctgagctcctcatcagtggaatggaaaagcccctccctctccgcacggatttctcttga
hSTING G230A/R293Q: Reference SNP (refSNP) Cluster Report: rs1131769 rs7380824 rs78233829
[SEQ ID NO: 330]
atgccccactccagcctgcatccatccatcccgtgtcccaggggtcacggggcccagaaggcagccttggttctgctgagtgcctgcc
tggtgaccctttgggggctaggagagccaccagagcacactctccggtacctggtgctccacctagcctccctgcagctgggactgct
gttaaacggggtctgcagcctggctgaggagctgcgccacatccactccaggtaccggggcagctactggaggactgtgcgggcct
gcctgggctgccccctccgccgtggggccctgttgctgctgtccatctatttctactactccctcccaaatgcggtcggcccgcccttcact
tggatgcttgccctcctgggcctctcgcaggcactgaacatcctcctgggcctcaagggcctggccccagctgagatctctgcagtgtgt
gaaaaagggaatttcaacgtggcccatgggctggcatggtcatattacatcggatatctgcggctgatcctgccagagctccaggccc
ggattcgaacttacaatcagcattacaacaacctgctacggggtgcagtgagccagcggctgtatattctcctcccattggactgtggg
gtgcctgataacctgagtatggctgaccccaacattcgcttcctggataaactgccccagcagaccgctgaccgtgctggcatcaagg
atcgggtttacagcaacagcatctatgagcttctggagaacgggcagcgggcgggcacctgtgtcctggagtacgccacccccttgc
agactttgtttgccatgtcacaatacagtcaagctggctttagccgggaggataggcttgagcaggccaaactcttctgccagacacttg
aggacatcctggcagatgcccctgagtctcagaacaactgccgcctcattgcctaccaggaacctgcagatgacagcagcttctcgct
gtcccaggaggttctccggcacctgcggcaggaggaaaaggaagaggttactgtgggcagcttgaagacctcagcggtgcccagt
acctccacgatgtcccaagagcctgagctcctcatcagtggaatggaaaagcccctccctctccgcacggatttctcttga
hSTING R71H/G230A/R293Q: Reference SNP (refSNP) Cluster Report:
rs1131769 rs7380824 rs78233829 rs11554776
[SEQ ID NO: 331]
atgccccactccagcctgcatccatccatcccgtgtcccaggggtcacggggcccagaaggcagccttggttctgctgagtgcctgcc
tggtgaccctttgggggctaggagagccaccagagcacactctccggtacctggtgctccacctagcctccctgcagctgggactgct
gttaaacggggtctgcagcctggctgaggagctgcaccacatccactccaggtaccggggcagctactggaggactgtgcgggcct
gcctgggctgccccctccgccgtggggccctgttgctgctgtccatctatttctactactccctcccaaatgcggtcggcccgcccttcact
tggatgcttgccctcctgggcctctcgcaggcactgaacatcctcctgggcctcaagggcctggccccagctgagatctctgcagtgtgt
gaaaaagggaatttcaacgtggcccatgggctggcatggtcatattacatcggatatctgcggctgatcctgccagagctccaggccc
ggattcgaacttacaatcagcattacaacaacctgctacggggtgcagtgagccagcggctgtatattctcctcccattggactgtggg
gtgcctgataacctgagtatggctgaccccaacattcgcttcctggataaactgccccagcagaccgctgaccgtgctggcatcaagg
atcgggtttacagcaacagcatctatgagcttctggagaacgggcagcgggcgggcacctgtgtcctggagtacgccacccccttgc
agactttgtttgccatgtcacaatacagtcaagctggctttagccgggaggataggcttgagcaggccaaactcttctgccagacacttg
aggacatcctggcagatgcccctgagtctcagaacaactgccgcctcattgcctaccaggaacctgcagatgacagcagcttctcgct
gtcccaggaggttctccggcacctgcggcaggaggaaaaggaagaggttactgtgggcagcttgaagacctcagcggtgcccagt
acctccacgatgtcccaagagcctgagctcctcatcagtggaatggaaaagcccctccctctccgcacggatttctcttga

The term “STING agonist”, as used herein, refers to a compound or antibody conjugate capable of binding to STING and activating STING. Activation of STING activity may include, for example, stimulation of inflammatory cytokines, including interferons, such as type 1 interferons, including IFN-α, IFN-β, type 3 interferons, e.g., IFNλ, IP10, TNF, IL-6, CXCL9, CCL4, CXCL11, CCL5, CCL3, or CCL8. STING agonist activity may also include stimulation of TANK binding kinase (TBK) 1 phosphorylation, interferon regulatory factor (IRF) activation (e.g., IRF3 activation), secretion of interferon-γ-inducible protein (IP-10), or other inflammatory proteins and cytokines. STING Agonist activity may be determined, for example, by the ability of a compound to stimulate activation of the STING pathway as detected using an interferon stimulation assay, a reporter gene assay (e.g., a hSTING wt assay, or a THP-1 Dual assay), a TBK1 activation assay, IP-10 assay, a STING Biochemical [3H]cGAMP Competition Assay, or other assays known to persons skilled in the art. STING Agonist activity may also be determined by the ability of a compound to increase the level of transcription of genes that encode proteins activated by STING or the STING pathway. Such activity may be detected, for example, using an RNAseq assay. In some embodiments, an assay to test for activity of a compound in a STING knock-out cell line may be used to determine if the compound is specific for STING, wherein a compound that is specific for STING would not be expected to have activity in a cell line wherein the STING pathway is partially or wholly deleted.

The term “TLR7 agonist”, as used herein, refers to a compound or antibody conjugate capable of activating Toll-like Receptor 7 (TLR7).

The terms “treat,” “treating” or “treatment,” as used herein, refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

The compound names provided herein were obtained using ChemDraw Ultra version 12.0 (CambridgeSoft®) or JChem version 5.3.1 (ChemAxon).

Unless specified otherwise, the term “compounds of the present invention”, “compounds of the invention” or “compounds provided herein” refers to compounds of Formula (I) and subformulae thereof (i.e. compounds of Formula (Ia) and Formula (Ib)), and pharmaceutically acceptable salts, stereoisomers (including diastereoisomers and enantiomers), tautomers and isotopically labeled compounds (including deuterium substitutions) thereof.

Unless specified otherwise, the term “antibody conjugate of the invention”, refers to antibody conjugates of Formula (II) and subformulae thereof (i.e. compounds of Formula (IIa) and Formula (IIb)), and pharmaceutically acceptable salts, stereoisomers (including diastereoisomers and enantiomers), tautomers and isotopically labeled compounds (including deuterium substitutions) thereof.

As used herein, the term “a,” “an,” “the” and similar terms used in the context of the present invention (especially in the context of the claims) are to be construed to cover both the singular and plural unless otherwise indicated herein or clearly contradicted by the context.

### I) Anti-DC-SIGN Antibodies

The present application discloses antibodies, or antibody fragments thereof (e.g., antigen binding fragments) that specifically bind to human DC-SIGN (anti-DC-SIGN antibody). DC-SIGN overexpression is observed in macrophages and dendritic cells in tumor microenvironment as well as lymphoid tissues and peripheral tissues. In some embodiments, the antibody or antibody fragment thereof that specifically binds to human DC-SIGN can be selected from the DC-SIGN antibodies disclosed herein.

Suitable anti-DC-SIGN monoclonal antibodies include, but are not limited to, the anti-DC-SIGN antibodies described in U.S. Pat. Nos. 7,534,866; 7,786,267; 7,846,744; 8,409,577; 8,779,107; 8,883,160; 8,916,696; PCT Publication Nos: WO2004091543; WO2005027979; WO2006066229; WO2006081576; WO2007046893; WO2008011599; WO2010053561; WO2011031736; WO2012145209; WO2013009841; WO2013024059; WO2013049307; WO2013095966; WO2013142255; WO2013125891; WO2013163689; WO2014064187; WO2014083499; WO2014144960; WO2014176604; WO2014179601; WO2015004473; WO2015023355; WO2015048633; WO2015048641; WO2015054039; WO2015073307; WO2015112626; U.S. Patent Publication No: US2014045242; and Chinese Patent Publication No: CN103739714, the contents of which are herein incorporated by reference in their entireties.

In some embodiments, the anti-DC-SIGN antibody or antibody fragment (e.g., an antigen binding fragment) comprises a VH domain having an amino acid sequence of any VH domain described in Table 1. Other suitable anti-DC-SIGN antibodies or antibody fragments (e.g., antigen binding fragments) can include amino acids that have been mutated, yet have at least 80, 85, 90, 95, 96, 97, 98, or 99 percent identity in the VH domain with the VH regions depicted in the sequences described in Table 1. The present disclosure in certain embodiments also provides antibodies or antibody fragments (e.g., antigen binding fragments) that specifically bind to DC-SIGN, wherein the antibodies or antibody fragments (e.g., antigen binding fragments) comprise a VH CDR having an amino acid sequence of any one of the VH CDRs listed in Table 1. In particular embodiments, the invention provides antibodies or antibody fragments (e.g., antigen binding fragments) that specifically bind to DC-SIGN, comprising (or alternatively, consist of) one, two, three, four, five or more VH CDRs having an amino acid sequence of any of the VH CDRs listed in Table 1.

In some embodiments, the anti-DC-SIGN antibody or antibody fragment (e.g., antigen binding fragments) comprises a VL domain having an amino acid sequence of any VL domain described in Table 1. Other suitable anti-DC-SIGN antibodies or antibody fragments (e.g., antigen binding fragments can include amino acids that have been mutated, yet have at least 80, 85, 90, 95, 96, 97, 98, or 99 percent identity in the VL domain with the VL regions depicted in the sequences described in Table 1. The present disclosure also provides antibodies or antibody fragments (e.g., antigen binding fragments) that specifically bind to DC-SIGN, the antibodies or antibody fragments (e.g., antigen binding fragments) comprise a VL CDR having an amino acid sequence of any one of the VL CDRs listed in Table 1. In particular, the invention provides antibodies or antibody fragments (e.g., antigen binding fragments) that specifically bind to DC-SIGN, which comprise (or alternatively, consist of) one, two, three or more VL CDRs having an amino acid sequence of any of the VL CDRs listed in Table 1.

Table 1. Sequences of exemplary anti-DC-SIGN monoclonal antibodies

TABLE 1
Sequences of exemplary anti-DC-SIGN monoclonal antibodies

| | | |
|---|---|---|
| >2B2Hz | | |
| SEQ ID NO: 1 | HCDR1 (Combined) | GYTFTNYGIN |
| SEQ ID NO: 2 | HCDR2 (Combined) | YIYIGNDYTEYNERFKG |
| SEQ ID NO: 3 | HCDR3 (Combined) | LYYGSSLYSYAMDY |
| SEQ ID NO: 4 | HCDR1 (Kabat) | NYGIN |
| SEQ ID NO: 2 | HCDR2 (Kabat) | YIYIGNDYTEYNERFKG |
| SEQ ID NO: 3 | HCDR3 (Kabat) | LYYGSSLYSYAMDY |
| SEQ ID NO: 5 | HCDR1 (Chothia) | GYTFTNY |
| SEQ ID NO: 6 | HCDR2 (Chothia) | YIGNDY |
| SEQ ID NO: 3 | HCDR3 (Chothia) | LYYGSSLYSYAMDY |
| SEQ ID NO: 7 | HCDR1 (IMGT) | GYTFTNYG |
| SEQ ID NO: 8 | HCDR2 (IMGT) | IYIGN |
| SEQ ID NO: 9 | HCDR3 (IMGT) | ARLYYGSSLYSYAMDY |
| SEQ ID NO: | VH | QVQLVQSGAEVKKPGASVKVSCKASGYTFTNYGINWVRQAPGQRLEWMGY |
| 10 | | IYIGNDYTEYNERFKGRVTITSDTSASTAYMELSSLRSEDTAVYYCARLYYGSSLY |
| | | SYAMDYWGQGTTVTVSS |
| SEQ ID NO: | DNA VH | CAAGTTCAGTTGGTTCAGTCTGGCGCCGAAGTGAAGAAACCTGGCGCCTC |
| 11 | | TGTGAAGGTGTCCTGCAAGGCTTCTGGCTACACCTTTACCAACTACGGCAT |
| | | CAACTGGGTCCGACAGGCTCCTGGCCAGAGATTGGAGTGGATGGGCTAC |
| | | ATCTACATCGGCAACGACTACACCGAGTACAACGAGCGGTTCAAGGGCAG |
| | | AGTGACCATCACCTCTGACACCTCTGCCTCCACCGCCTACATGGAACTGTC |
| | | CAGCCTGAGATCTGAGGACACCGCCGTGTACTACTGCGCCAGGCTGTACT |
| | | ATGGCTCCTCCCTGTACAGCTATGCCATGGACTACTGGGGACAGGGCACA |
| | | ACCGTGACAGTGAGCTCC |
| SEQ ID NO: | Heavy | QVQLVQSGAEVKKPGASVKVSCKASGYTFTNYGINWVRQAPGQRLEWMGY |
| 12 | Chain | IYIGNDYTEYNERFKGRVTITSDTSASTAYMELSSLRSEDTAVYYCARLYYGSSLY |
| | | SYAMDYWGQGTTVTVSSASTKGPSVFPLAPSSKSTSGGTAALGCLVKDYFPC |
| | | PVTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVSVLTVLH |
| | | QDWLNGKEYKCKVSNKALAAPIEKTISKAKGQPREPQVYTLPPSREEMTKNQ |
| | | VSLTCLVKGFYPCDIAVEWESNGQPENNYKTTPPVLDSDGSFFLYSKLTVDKSR |
| | | WQQGNVFSCSVMHEALHNHYTQKSLSLSPGK |
| SEQ ID NO: | DNA Heavy | CAAGTTCAGTTGGTTCAGTCTGGCGCCGAAGTGAAGAAACCTGGCGCCTC |
| 13 | Chain | TGTGAAGGTGTCCTGCAAGGCTTCTGGCTACACCTTTACCAACTACGGCAT |
| | | CAACTGGGTCCGACAGGCTCCTGGCCAGAGATTGGAGTGGATGGGCTAC |
| | | ATCTACATCGGCAACGACTACACCGAGTACAACGAGCGGTTCAAGGGCAG |
| | | AGTGACCATCACCTCTGACACCTCTGCCTCCACCGCCTACATGGAACTGTC |
| | | CAGCCTGAGATCTGAGGACACCGCCGTGTACTACTGCGCCAGGCTGTACT |
| | | ATGGCTCCTCCCTGTACAGCTATGCCATGGACTACTGGGGACAGGGCACA |
| | | ACCGTGACAGTGAGCTCCGCTAGCACCAAGGGCCCAAGTGTGTTTCCCCT |
| | | GGCCCCCAGCAGCAAGTCTACTTCCGGCGGAACTGCTGCCCTGGGTTGCC |
| | | TGGTGAAGGACTACTTCCCCTGTCCCGTGACAGTGTCCTGGAACTCTGGG |
| | | GCTCTGACTTCCGGCGTGCACACCTTCCCCGCCGTGCTGCAGAGCAGCGG |
| | | CCTGTACAGCCTGAGCAGCGTGGTGACAGTGCCCTCCAGCTCTCTGGGAA |
| | | CCCAGACCTATATCTGCAACGTGAACCACAAGCCCAGCAACACCAAGGTG |
| | | GACAAGAGAGTGGAGCCCAAGAGCTGCGACAAGACCCACACCTGCCCCC |
| | | CCTGCCCAGCTCCAGAACTGCTGGGAGGGCCTTCCGTGTTCCTGTTCCCCC |
| | | CCAAGCCCAAGGACACCCTGATGATCAGCAGGACCCCCGAGGTGACCTGC |
| | | GTGGTGGTGGCCGTGTCCCACGAGGACCCAGAGGTGAAGTTCAACTGGT |
| | | ACGTGGACGGCGTGGAGGTGCACAACGCCAAGACCAAGCCCAGAGAGGA |
| | | GCAGTACAACAGCACCTACAGGGTGGTGTCCGTGCTGACCGTGCTGCACC |
| | | AGGACTGGCTGAACGGCAAAGAATACAAGTGCAAAGTCTCCAACAAGGC |
| | | CCTGGCTGCCCCAATCGAAAAGACAATCAGCAAGGCCAAGGGCCAGCCAC |
| | | GGGAGCCCCAGGTGTACACCCTGCCCCCCAGCCGGGAGGAGATGACCAA |
| | | GAACCAGGTGTCCCTGACCTGTCTGGTGAAGGGCTTCTACCCCTGTGATAT |
| | | CGCCGTGGAGTGGGAGAGCAACGGCCAGCCCGAGAACAACTACAAGACC |
| | | ACCCCCCCAGTGCTGGACAGCGACGGCAGCTTCTTCCTGTACAGCAAGCT |
| | | GACCGTGGACAAGTCCAGGTGGCAGCAGGGCAACGTGTTCAGCTGCAGC |
| | | GTGATGCACGAGGCCCTGCACAACCACTACACCCAGAAGTCCCTGAGCCT |
| | | GAGCCCCGGCAAG |
| SEQ ID NO: 14 | LCDR1 (Combined) | RSSKSLLHSSGNTYLY |
| SEQ ID NO: 15 | LCDR2 (Combined) | RMSNLAS |
| SEQ ID NO: 16 | LCDR3 (Combined) | MQHLEYPYT |
| SEQ ID NO: 14 | LCDR1 (Kabat) | RSSKSLLHSSGNTYLY |
| SEQ ID NO: 15 | LCDR2 (Kabat) | RMSNLAS |
| SEQ ID NO: 16 | LCDR3 (Kabat) | MQHLEYPYT |
| SEQ ID NO: 17 | LCDR1 (Chothia) | SKSLLHSSGNTY |
| SEQ ID NO: 18 | LCDR2 (Chothia) | RMS |
| SEQ ID NO: 19 | LCDR3 (Chothia) | HLEYPY |
| SEQ ID NO: 20 | LCDR1 (IMGT) | KSLLHSSGNTY |
| SEQ ID NO: 18 | LCDR2 (IMGT) | RMS |
| SEQ ID NO: 16 | LCDR3 (IMGT) | MQHLEYPYT |
| SEQ ID NO: 21 | VL | DIVMTQSPLSLPVTPGEPASISCRSSKSLLHSSGNTYLYWFLQKPGQSPQLLISR |
| | | MSNLASGVPDRFSGSGSGTDFTLKISRVEAEDVGVYYCMQHLEYPYTFGGGT |
| | | KVEIK |
| SEQ ID NO: 22 | DNA VL | GACATTGTGATGACCCAGTCTCCACTGAGCCTGCCTGTGACACCTGGCGA |
| | | GCCTGCTTCCATCTCCTGCCGGTCCTCTAAGTCCCTGCTGCACTCTTCCGGC |
| | | AATACCTACCTGTACTGGTTCCTGCAGAAGCCCGGCCAGTCTCCTCAGCTG |
| | | CTGATCTCCAGAATGTCCAACCTGGCCTCTGGCGTGCCCGACAGATTTTCT |
| | | GGCTCTGGATCTGGCACCGACTTCACCCTGAAGATCTCTAGAGTGGAAGC |
| | | CGAGGACGTGGGCGTGTACTACTGTATGCAGCACCTGGAATACCCCTACA |
| | | CCTTCGGCGGAGGCACCAAGGTGGAAATCAAG |
| SEQ ID NO: 23 | Light Chain | DIVMTQSPLSLPVTPGEPASISCRSSKSLLHSSGNTYLYWFLQKPGQSPQLLISR |
| | | MSNLASGVPDRFSGSGSGTDFTLKISRVEAEDVGVYYCMQHLEYPYTFGGGT |
| | | KVEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKSF |
| | | NRGEC |
| SEQ ID NO: 24 | DNA Light | GACATTGTGATGACCCAGTCTCCACTGAGCCTGCCTGTGACACCTGGCGA |
| | Chain | GCCTGCTTCCATCTCCTGCCGGTCCTCTAAGTCCCTGCTGCACTCTTCCGGC |
| | | AATACCTACCTGTACTGGTTCCTGCAGAAGCCCGGCCAGTCTCCTCAGCTG |
| | | CTGATCTCCAGAATGTCCAACCTGGCCTCTGGCGTGCCCGACAGATTTTCT |
| | | GGCTCTGGATCTGGCACCGACTTCACCCTGAAGATCTCTAGAGTGGAAGC |
| | | CGAGGACGTGGGCGTGTACTACTGTATGCAGCACCTGGAATACCCCTACA |
| | | CCTTCGGCGGAGGCACCAAGGTGGAAATCAAGCGTACGGTGGCCGCTCCC |
| | | AGCGTGTTCATCTTCCCCCCCAGCGACGAGCAGCTGAAGAGTGGCACCGC |
| | | CAGCGTGGTGTGCCTGCTGAACAACTTCTACCCCCGGGAGGCCAAGGTGC |
| | | AGTGGAAGGTGGACAACGCCCTGCAGAGCGGCAACAGCCAGGAGAGCGT |
| | | CACCGAGCAGGACAGCAAGGACTCCACCTACAGCCTGAGCAGCACCCTGA |
| | | CCCTGAGCAAGGCCGACTACGAGAAGCATAAGGTGTACGCCTGCGAGGT |
| | | GACCCACCAGGGCCTGTCCAGCCCCGTGACCAAGAGCTTCAACAGGGGCG |
| | | AGTGC |
| 960K03 N92S | | |
| SEQ ID NO: 25 | HCDR1 (Combined) | GFSLSTGGMSVS |
| SEQ ID NO: 26 | HCDR2 (Combined) | LIDWDDDKYYSTSLKT |
| SEQ ID NO: 27 | HCDR3 (Combined) | AHSGSYFDF |
| SEQ ID NO: 28 | HCDR1 (Kabat) | TGGMSVS |
| SEQ ID NO: 26 | HCDR2 (Kabat) | LIDWDDDKYYSTSLKT |
| SEQ ID NO: 27 | HCDR3 (Kabat) | AHSGSYFDF |
| SEQ ID NO: 29 | HCDR1 (Chothia) | GFSLSTGGM |
| SEQ ID NO: 30 | HCDR2 (Chothia) | DWDDD |
| SEQ ID NO: 27 | HCDR3 (Chothia) | AHSGSYFDF |
| SEQ ID NO: 31 | HCDR1 (IMGT) | GFSLSTGGMS |
| SEQ ID NO: 32 | HCDR2 (IMGT) | IDWDDDK |
| SEQ ID NO: 33 | HCDR3 (IMGT) | ARAHSGSYFDF |
| SEQ ID NO: 34 | VH | QVTLRESGPALVKPTQTLTLTCTFSGFSLSTGGMSVSWIRQPPGKALEWLALI |
| | | DWDDDKYYSTSLKTRLTISKDTSKNQLVLTMTNMDPVDTATYYCARAHSGSY |
| | | FDFWGQGTLVTVSS |
| SEQ ID NO: 35 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCA |
| SEQ ID NO: | Heavy | QVTLRESGPALVKPTQTLTLTCTFSGFSLSTGGMSVSWIRQPPGKALEWLALI |
| 36 | Chain | DWDDDKYYSTSLKTRLTISKDTSKNQLVLTMTNMDPVDTATYYCARAHSGSY |
| | | FDFWGQGTLVTVSSASTKGPSVFPLAPSSKSTSGGTAALGCLVKDYFPCPVTV |
| | | SWNSGALTSGVHTFPAVLQSSGLYSLSSVVIVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLIVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 37 | Chain | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCAGCCTCCACCAAGGGCCCATCGGTGTTTCCCCTGGCCCCCAGCAGCAA |
| | | GTCTACTTCCGGCGGAACTGCTGCCCTGGGTTGCCTGGTGAAGGACTACTT |
| | | CCCCTGTCCCGTGACAGTGTCCTGGAACTCTGGGGCTCTGACTTCCGGCGT |
| | | GCACACCTTCCCCGCCGTGCTGCAGAGCAGCGGCCTGTACAGCCTGAGCA |
| | | GCGTGGTGACAGTGCCCTCCAGCTCTCTGGGAACCCAGACCTATATCTGCA |
| | | ACGTGAACCACAAGCCCAGCAACACCAAGGTGGACAAGAGAGTGGAGCC |
| | | CAAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: 38 | LCDR1 (Combined) | RASQRISNWLA |
| SEQ ID NO: 39 | LCDR2 (Combined) | KASSLES |
| SEQ ID NO: 40 | LCDR3 (Combined) | QQFSSYWT |
| SEQ ID NO: 38 | LCDR1 (Kabat) | RASQRISNWLA |
| SEQ ID NO: 39 | LCDR2 (Kabat) | KASSLES |
| SEQ ID NO: 40 | LCDR3 (Kabat) | QQFSSYWT |
| SEQ ID NO: 41 | LCDR1 (Chothia) | SQRISNW |
| SEQ ID NO: 42 | LCDR2 (Chothia) | KAS |
| SEQ ID NO: 43 | LCDR3 (Chothia) | FSSYW |
| SEQ ID NO: 44 | LCDR1 (IMGT) | QRISNW |
| SEQ ID NO: 42 | LCDR2 (IMGT) | KAS |
| SEQ ID NO: 40 | LCDR3 (IMGT) | QQFSSYWT |
| SEQ ID NO: 45 | VL | DIQMTQSPSTLSASVGDRVTITCRASQRISNWLAWYQQKPGKAPKLLIYKASS |
| | | LESGVPSRFSGSGSGTEFTLTISSLQPDDFATYYCQQFSSYWTFGQGTKVEIK |
| SEQ ID NO: 46 | DNA VL | GACATCCAGATGACCCAGTCTCCTTCCACCCTGTCTGCATCTGTAGGAGAC |
| | | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAGTAGTTATTGGACGTTCGGCCAAGGGACC |
| | | AAGGTGGAAATCAAA |
| SEQ ID NO: 47 | Light Chain | DIQMTQSPSTLSASVGDRVTITCRASQRISWLAWYQQKPGKAPKLLIYKASS |
| | | LESGVPSRFSGSGSGTEFTLTISSLQPDDFATYYCQQFSSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 48 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAGTAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 958N05 S93A | | |
| SEQ ID NO: 49 | HCDR1 (Combined) | GFSLSTSGISVS |
| SEQ ID NO: 26 | HCDR2 (Combined) | LIDWDDDKYYSTSLKT |
| SEQ ID NO: 50 | HCDR3 (Combined) | TPSGSYGRYFDL |
| SEQ ID NO: 51 | HCDR1 (Kabat) | TSGISVS |
| SEQ ID NO: 26 | HCDR2 (Kabat) | LIDWDDDKYYSTSLKT |
| SEQ ID NO: 50 | HCDR3 (Kabat) | TPSGSYGRYFDL |
| SEQ ID NO: 52 | HCDR1 (Chothia) | GFSLSTSGI |
| SEQ ID NO: 30 | HCDR2 (Chothia) | DWDDD |
| SEQ ID NO: 50 | HCDR3 (Chothia) | TPSGSYGRYFDL |
| SEQ ID NO: 53 | HCDR1 (IMGT) | GFSLSTSGIS |
| SEQ ID NO: 32 | HCDR2 (IMGT) | IDWDDDK |
| SEQ ID NO: 54 | HCDR3 (IMGT) | ARTPSGSYGRYFDL |
| SEQ ID NO: 55 | VH | QVTLRESGPALVKPTQTLTLTCTFSGFSLSTSGISVSWIRQPPGKALEWLALID |
| | | WDDDKYYSTSLKTRLTISKDTSKNQVVLTMTNMDPVDTATYYCARTPSGSYG |
| | | RYFDLWGRGTLVTVSS |
| SEQ ID NO: 56 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCA |
| SEQ ID NO: 57 | Heavy | QVTLRESGPALVKPTQTLTLTCTFSGFSLSTSGISVSWIRQPPGKALEWLALID |
| | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 58 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 59 | LCDR1 (Combined) | RASQSISNWLA |
| SEQ ID NO: 39 | LCDR2 (Combined) | KASSLES |
| SEQ ID NO: 60 | LCDR3 (Combined) | QQYNAYWT |
| SEQ ID NO: 59 | LCDR1 (Kabat) | RASQSISNWLA |
| SEQ ID NO: 39 | LCDR2 (Kabat) | KASSLES |
| SEQ ID NO: 60 | LCDR3 (Kabat) | QQYNAYWT |
| SEQ ID NO: 61 | LCDR1 (Chothia) | SQSISNW |
| SEQ ID NO: 42 | LCDR2 (Chothia) | KAS |
| SEQ ID NO: 62 | LCDR3 (Chothia) | YNAYW |
| SEQ ID NO: 63 | LCDR1 (IMGT) | QSISNW |
| SEQ ID NO: 42 | LCDR2 (IMGT) | KAS |
| SEQ ID NO: 60 | LCDR3 (IMGT) | QQYNAYWT |
| SEQ ID NO: 64 | VL | DIQLTQSPSTLSASVGDRVTITCRASQSISNWLAWYQQKPGKAPKLLIYKASSL |
| | | ESGVPSRFTGSGSGTEFTLTISSLQPDDFATYYCQQYNAYWTFGQGTKVEIK |
| SEQ ID NO: 65 | DNA VL | GACATCCAGTTGACCCAGTCTCCTTCCACCCTGTCTGCATCTGTAGGAGAC |
| | | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATGCCTATTGGACGTTCGGCCAAGGGACC |
| | | AAGGTGGAAATCAAA |
| SEQ ID NO: 66 | Light Chain | DIQLTQSPSTLSASVGDRVTITCRASQSISNWLAWYQQKPGKAPKLLIYKASSL |
| | | ESGVPSRFTGSGSGTEFTLTISSLQPDDFATYYCQQYNAYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 67 | DNA Light | GACATCCAGTTGACCCAGTCTCCTTCCACCCTGTCTGCATCTGTAGGAGAC |
| | Chain | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATGCCTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 960K03 | | |
| N92Q | | |
| SEQ ID NO: 25 | HCDR1 (Combined) | GFSLSTGGMSVS |
| SEQ ID NO: 26 | HCDR2 (Combined) | LIDWDDDKYYSTSLKT |
| SEQ ID NO: 27 | HCDR3 (Combined) | AHSGSYFDF |
| SEQ ID NO: 28 | HCDR1 (Kabat) | TGGMSVS |
| SEQ ID NO: 26 | HCDR2 (Kabat) | LIDWDDDKYYSTSLKT |
| SEQ ID NO: 27 | HCDR3 (Kabat) | AHSGSYFDF |
| SEQ ID NO: 29 | HCDR1 (Chothia) | GFSLSTGGM |
| SEQ ID NO: 30 | HCDR2 (Chothia) | DWDDD |
| SEQ ID NO: 27 | HCDR3 (Chothia) | AHSGSYFDF |
| SEQ ID NO: 31 | HCDR1 (IMGT) | GFSLSTGGMS |
| SEQ ID NO: 32 | HCDR2 (IMGT) | IDWDDDK |
| SEQ ID NO: 33 | HCDR3 (IMGT) | ARAHSGSYFDF |
| SEQ ID NO: 34 | VH | QVTLRESGPALVKPTQTLTLTCTFSGFSLSTGGMSVSWIRQPPGKALEWLALI |
| | | DWDDDKYYSTSLKTRLTISKDTSKNQLVLTMTNMDPVDTATYYCARAHSGSY |
| | | FDFWGQGTLVTVSS |
| SEQ ID NO: 35 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCA |
| SEQ ID NO: 36 | Heavy | QVTLRESGPALVKPTQTLTLTCTFSGFSLSTGGMSVSWIRQPPGKALEWLALI |
| | Chain | DWDDDKYYSTSLKTRLTISKDTSKNQLVLTMTNMDPVDTATYYCARAHSGSY |
| | | FDFWGQGTLVTVSSASTKGPSVFPLAPSSKSTSGGTAALGCLVKDYFPCPVTV |
| | | SWNSGALTSGVHTFPAVLQSSGLYSLSSVVIVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLIVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: 37 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCAGCCTCCACCAAGGGCCCATCGGTGTTTCCCCTGGCCCCCAGCAGCAA |
| | | GTCTACTTCCGGCGGAACTGCTGCCCTGGGTTGCCTGGTGAAGGACTACTT |
| | | CCCCTGTCCCGTGACAGTGTCCTGGAACTCTGGGGCTCTGACTTCCGGCGT |
| | | GCACACCTTCCCCGCCGTGCTGCAGAGCAGCGGCCTGTACAGCCTGAGCA |
| | | GCGTGGTGACAGTGCCCTCCAGCTCTCTGGGAACCCAGACCTATATCTGCA |
| | | ACGTGAACCACAAGCCCAGCAACACCAAGGTGGACAAGAGAGTGGAGCC |
| | | CAAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: 38 | LCDR1 (Combined) | RASQRISNWLA |
| SEQ ID NO: 39 | LCDR2 (Combined) | KASSLES |
| SEQ ID NO: 68 | LCDR3 (Combined) | QQFQSYWT |
| SEQ ID NO: 38 | LCDR1 (Kabat) | RASQRISNWLA |
| SEQ ID NO: 39 | LCDR2 (Kabat) | KASSLES |
| SEQ ID NO: 68 | LCDR3 (Kabat) | QQFQSYWT |
| SEQ ID NO: 41 | LCDR1 (Chothia) | SQRISNW |
| SEQ ID NO: 42 | LCDR2 (Chothia) | KAS |
| SEQ ID NO: 69 | LCDR3 (Chothia) | FQSYW |
| SEQ ID NO: 44 | LCDR1 (IMGT) | QRISNW |
| SEQ ID NO: 42 | LCDR2 (IMGT) | KAS |
| SEQ ID NO: 68 | LCDR3 (IMGT) | QQFQSYWT |
| SEQ ID NO: 70 | VL | DIQMTQSPSTLSASVGDRVTITCRASQRISNWLAWYQQKPGKAPKLLIYKASS |
| | | LESGVPSRFSGSGSGTEFTLTISSLQPDDFATYYCQQFQSYWTFGQGTKVEIK |
| SEQ ID NO: 71 | DNA VL | GACATCCAGATGACCCAGTCTCCTTCCACCCTGTCTGCATCTGTAGGAGAC |
| | | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTCAGAGTTATTGGACGTTCGGCCAAGGGACC |
| | | AAGGTGGAAATCAAA |
| SEQ ID NO: 72 | Light Chain | DIQMTQSPSTLSASVGDRVTITCRASQRISNWLAWYQQKPGKAPKLLIYKASS |
| | | LESGVPSRFSGSGSGTEFTLTISSLQPDDFATYYCQQFQSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 73 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTCAGAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 952P16 | | |
| N92Q | | |
| SEQ ID NO: 74 | HCDR1 (Combined) | GFSLSTSGVSVS |
| SEQ ID NO: 26 | HCDR2 (Combined) | LIDWDDDKYYSTSLKT |
| SEQ ID NO: 50 | HCDR3 (Combined) | TPSGSYGRYFDL |
| SEQ ID NO: 75 | HCDR1 (Kabat) | TSGVSVS |
| SEQ ID NO: 26 | HCDR2 (Kabat) | LIDWDDDKYYSTSLKT |
| SEQ ID NO: 50 | HCDR3 (Kabat) | TPSGSYGRYFDL |
| SEQ ID NO: 76 | HCDR1 (Chothia) | GFSLSTSGV |
| SEQ ID NO: 30 | HCDR2 (Chothia) | DWDDD |
| SEQ ID NO: 50 | HCDR3 (Chothia) | TPSGSYGRYFDL |
| SEQ ID NO: 77 | HCDR1 (IMGT) | GFSLSTSGVS |
| SEQ ID NO: 32 | HCDR2 (IMGT) | IDWDDDK |
| SEQ ID NO: 54 | HCDR3 (IMGT) | ARTPSGSYGRYFDL |
| SEQ ID NO: 78 | VH | QVTLRESGPALVKPTQTLTLTCTFSGFSLSTSGVSVSWIRQPPGKALEWLALID |
| | | WDDDKYYSTSLKTRLTISKDTSKNQVVLTMTNMDPVDTATYYCARTPSGSYG |
| | | RYFDLWGRGTLVTVSS |
| SEQ ID NO: 79 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: 80 | Heavy | QVTLRESGPALVKPTQTLTLTCTFSGFSLSTSGVSVSWIRQPPGKALEWLALID |
| | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 81 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 59 | LCDR1 (Combined) | RASQSISNWLA |
| SEQ ID NO: 39 | LCDR2 (Combined) | KASSLES |
| SEQ ID NO: 82 | LCDR3 (Combined) | QQYQSYWT |
| SEQ ID NO: 59 | LCDR1 (Kabat) | RASQSISNWLA |
| SEQ ID NO: 39 | LCDR2 (Kabat) | KASSLES |
| SEQ ID NO: 82 | LCDR3 (Kabat) | QQYQSYWT |
| SEQ ID NO: 61 | LCDR1 (Chothia) | SQSISNW |
| SEQ ID NO: 42 | LCDR2 (Chothia) | KAS |
| SEQ ID NO: 83 | LCDR3 (Chothia) | YQSYW |
| SEQ ID NO: 63 | LCDR1 (IMGT) | QSISNW |
| SEQ ID NO: 42 | LCDR2 (IMGT) | KAS |
| SEQ ID NO: 82 | LCDR3 (IMGT) | QQYQSYWT |
| SEQ ID NO: 84 | VL | DIQMTQSPSTLSASVGDRVTITCRASQSISNWLAWYQQKPGKAPKLLIYKASS |
| | | LESGVPSRFSGSGSGTEFTLTISSLQPDDFATYYCQQYQSYWTFGQGTKVEIK |
| SEQ ID NO: 85 | DNA VL | GACATCCAGATGACCCAGTCTCCTTCCACCCTGTCTGCATCTGTAGGAGAC |
| | | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAA |
| SEQ ID NO: 86 | Light Chain | DIQMTQSPSTLSASVGDRVTITCRASQSISNWLAWYQQKPGKAPKLLIYKASS |
| | | LESGVPSRFSGSGSGTEFTLTISSLQPDDFATYYCQQYQSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 87 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 952G04 | | |
| N92Q | | |
| SEQ ID NO: 88 | HCDR1 (Combined) | GFSLSTSGVSVT |
| SEQ ID NO: 26 | HCDR2 (Combined) | LIDWDDDKYYSTSLKT |
| SEQ ID NO: 50 | HCDR3 (Combined) | TPSGSYGRYFDL |
| SEQ ID NO: 89 | HCDR1 (Kabat) | TSGVSVT |
| SEQ ID NO: 26 | HCDR2 (Kabat) | LIDWDDDKYYSTSLKT |
| SEQ ID NO: 50 | HCDR3 (Kabat) | TPSGSYGRYFDL |
| SEQ ID NO: 76 | HCDR1 (Chothia) | GFSLSTSGV |
| SEQ ID NO: 30 | HCDR2 (Chothia) | DWDDD |
| SEQ ID NO: 50 | HCDR3 (Chothia) | TPSGSYGRYFDL |
| SEQ ID NO: 77 | HCDR1 (IMGT) | GFSLSTSGVS |
| SEQ ID NO: 32 | HCDR2 (IMGT) | IDWDDDK |
| SEQ ID NO: 54 | HCDR3 (IMGT) | ARTPSGSYGRYFDL |
| SEQ ID NO: 90 | VH | QVTLRESGPALVKPTQTLTLTCTFSGFSLSTSGVSVTWIRQPPGKALEWLALID |
| | | WDDDKYYSTSLKTRLAISKDTSKNQVVLTMTNMDPVDTATYYCARTPSGSYG |
| | | RYFDLWGRGTLVTVSS |
| SEQ ID NO: 91 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: 92 | Heavy | QVTLRESGPALVKPTQTLTLTCTFSGFSLSTSGVSVTWIRQPPGKALEWLALID |
| | Chain | WDDDKYYSTSLKTRLAISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 93 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 94 | LCDR1 (Combined) | RASQSISTWLA |
| SEQ ID NO: 95 | LCDR2 (Combined) | EASSLES |
| SEQ ID NO: 82 | LCDR3 (Combined) | QQYQSYWT |
| SEQ ID NO: 94 | LCDR1 (Kabat) | RASQSISTWLA |
| SEQ ID NO: 95 | LCDR2 (Kabat) | EASSLES |
| SEQ ID NO: 82 | LCDR3 (Kabat) | QQYQSYWT |
| SEQ ID NO: 96 | LCDR1 (Chothia) | SQSISTW |
| SEQ ID NO: 97 | LCDR2 (Chothia) | EAS |
| SEQ ID NO: 83 | LCDR3 (Chothia) | YQSYW |
| SEQ ID NO: 98 | LCDR1 (IMGT) | QSISTW |
| SEQ ID NO: 97 | LCDR2 (IMGT) | EAS |
| SEQ ID NO: 82 | LCDR3 (IMGT) | QQYQSYWT |
| SEQ ID NO: 99 | VL | DIQMTQSPSTLSASVGDRVTITCRASQSISTWLAWYQQKPGKAPKLLIYEASSL |
| | | ESGVPSRFSGSGSGTEFTLTISSLQPDDFATYYCQQYQSYWTFGQGTKVEIK |
| SEQ ID NO: 100 | DNA VL | GACATCCAGATGACCCAGTCTCCTTCCACCCTGTCTGCATCTGTAGGAGAC |
| | | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAA |
| SEQ ID NO: 101 | Light Chain | DIQMTQSPSTLSASVGDRVTITCRASQSISTWLAWYQQKPGKAPKLLIYEASSL |
| | | ESGVPSRFSGSGSGTEFTLTISSLQPDDFATYYCQQYQSYWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| SEQ ID NO: 102 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 2B2 Chimeric | | |
| SEQ ID NO: 1 | HCDR1 (Combined) | GYTFTNYGIN |
| SEQ ID NO: 2 | HCDR2 (Combined) | YIYIGNDYTEYNERFKG |
| SEQ ID NO: 3 | HCDR3 (Combined) | LYYGSSLYSYAMDY |
| SEQ ID NO: 4 | HCDR1 (Kabat) | NYGIN |
| SEQ ID NO: 2 | HCDR2 (Kabat) | YIYIGNDYTEYNERFKG |
| SEQ ID NO: 3 | HCDR3 (Kabat) | LYYGSSLYSYAMDY |
| SEQ ID NO: 5 | HCDR1 (Chothia) | GYTFTNY |
| SEQ ID NO: 6 | HCDR2 (Chothia) | YIGNDY |
| SEQ ID NO: 3 | HCDR3 (Chothia) | LYYGSSLYSYAMDY |
| SEQ ID NO: 7 | HCDR1 (IMGT) | GYTFTNYG |
| SEQ ID NO: 8 | HCDR2 (IMGT) | IYIGN |
| SEQ ID NO: 9 | HCDR3 (IMGT) | ARLYYGSSLYSYAMDY |
| SEQ ID NO: 103 | VH | EVQLQQSGAELVRPGSSVKMSCKTSGYTFTNYGINWVKQRPGQGLEWIGYIY |
| | | IGNDYTEYNERFKGKATLTSDTSSSTAHIQLNSLTSEDSAIYFCARLYYGSSLYSY |
| | | AMDYWGQGTSVTVSS |
| SEQ ID NO: 104 | DNA VH | GAGGTTCAGCTGCAGCAGTCTGGAGCTGAGTTGGTGAGGCCTGGGTCCTC |
| | | AGTGAAGATGTCCTGCAAGACTTCTGGATATACATTCACAAACTACGGTAT |
| | | AAACTGGGTGAAGCAGAGGCCTGGACAGGGCCTGGAATGGATTGGATAT |
| | | ATTTATATTGGAAATGATTATACTGAGTACAATGAGAGGTTCAAGGGCAA |
| | | GGCCACACTGACTTCAGACACATCCTCCAGCACAGCCCACATACAACTCAA |
| | | CAGCCTGACATCTGAGGACTCTGCAATCTATTTCTGTGCAAGACTTTACTAC |
| | | GGTAGTAGCCTCTATTCTTATGCTATGGACTACTGGGGTCAAGGAACCTCT |
| | | GTCACAGTCTCCTCT |
| SEQ ID NO: 105 | Heavy | EVQLQQSGAELVRPGSSVKMSCKTSGYTFTNYGINWVKQRPGQGLEWIGYIY |
| | Chain | IGNDYTEYNERFKGKATLTSDTSSSTAHIQLNSLTSEDSAIVFCARLYYGSSLYSY |
| | | AMDYWGQGTSVTVSSASTKGPSVFPLAPSSKSTSGGTAALGCLVKDYFPCPV |
| | | TVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTC |
| | | VVVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 106 | DNA Heavy | GAGGTTCAGCTGCAGCAGTCTGGAGCTGAGTTGGTGAGGCCTGGGTCCTC |
| | Chain | AGTGAAGATGTCCTGCAAGACTTCTGGATATACATTCACAAACTACGGTAT |
| | | AAACTGGGTGAAGCAGAGGCCTGGACAGGGCCTGGAATGGATTGGATAT |
| | | ATTTATATTGGAAATGATTATACTGAGTACAATGAGAGGTTCAAGGGCAA |
| | | GGCCACACTGACTTCAGACACATCCTCCAGCACAGCCCACATACAACTCAA |
| | | CAGCCTGACATCTGAGGACTCTGCAATCTATTTCTGTGCAAGACTTTACTAC |
| | | GGTAGTAGCCTCTATTCTTATGCTATGGACTACTGGGGTCAAGGAACCTCT |
| | | GTCACAGTCTCCTCTGCTAGCACCAAGGGCCCAAGTGTGTTTCCCCTGGCC |
| | | CCCAGCAGCAAGTCTACTTCCGGCGGAACTGCTGCCCTGGGTTGCCTGGT |
| | | GAAGGACTACTTCCCCTGTCCCGTGACAGTGTCCTGGAACTCTGGGGCTCT |
| | | GACTTCCGGCGTGCACACCTTCCCCGCCGTGCTGCAGAGCAGCGGCCTGT |
| | | ACAGCCTGAGCAGCGTGGTGACAGTGCCCTCCAGCTCTCTGGGAACCCAG |
| | | ACCTATATCTGCAACGTGAACCACAAGCCCAGCAACACCAAGGTGGACAA |
| | | GAGAGTGGAGCCCAAGAGCTGCGACAAGACCCACACCTGCCCCCCCTGCC |
| | | CAGCTCCAGAACTGCTGGGAGGGCCTTCCGTGTTCCTGTTCCCCCCCAAGC |
| | | CCAAGGACACCCTGATGATCAGCAGGACCCCCGAGGTGACCTGCGTGGTG |
| | | GTGGCCGTGTCCCACGAGGACCCAGAGGTGAAGTTCAACTGGTACGTGG |
| | | ACGGCGTGGAGGTGCACAACGCCAAGACCAAGCCCAGAGAGGAGCAGTA |
| | | CAACAGCACCTACAGGGTGGTGTCCGTGCTGACCGTGCTGCACCAGGACT |
| | | GGCTGAACGGCAAAGAATACAAGTGCAAAGTCTCCAACAAGGCCCTGGCT |
| | | GCCCCAATCGAAAAGACAATCAGCAAGGCCAAGGGCCAGCCACGGGAGC |
| | | CCCAGGTGTACACCCTGCCCCCCAGCCGGGAGGAGATGACCAAGAACCAG |
| | | GTGTCCCTGACCTGTCTGGTGAAGGGCTTCTACCCCTGTGATATCGCCGTG |
| | | GAGTGGGAGAGCAACGGCCAGCCCGAGAACAACTACAAGACCACCCCCC |
| | | CAGTGCTGGACAGCGACGGCAGCTTCTTCCTGTACAGCAAGCTGACCGTG |
| | | GACAAGTCCAGGTGGCAGCAGGGCAACGTGTTCAGCTGCAGCGTGATGC |
| | | ACGAGGCCCTGCACAACCACTACACCCAGAAGTCCCTGAGCCTGAGCCCC |
| | | GGCAAG |
| SEQ ID NO: 14 | LCDR1 (Combined) | RSSKSLLHSSGNTYLY |
| SEQ ID NO: 15 | LCDR2 (Combined) | RMSNLAS |
| SEQ ID NO: 16 | LCDR3 (Combined) | MQHLEYPYT |
| SEQ ID NO: 14 | LCDR1 (Kabat) | RSSKSLLHSSGNTYLY |
| SEQ ID NO: 15 | LCDR2 (Kabat) | RMSNLAS |
| SEQ ID NO: 16 | LCDR3 (Kabat) | MQHLEYPYT |
| SEQ ID NO: 17 | LCDR1 (Chothia) | SKSLLHSSGNTY |
| SEQ ID NO: 18 | LCDR2 (Chothia) | RMS |
| SEQ ID NO: 19 | LCDR3 (Chothia) | HLEYPY |
| SEQ ID NO: 20 | LCDR1 (IMGT) | KSLLHSSGNTY |
| SEQ ID NO: 18 | LCDR2 (IMGT) | RMS |
| SEQ ID NO: 16 | LCDR3 (IMGT) | MQHLEYPYT |
| SEQ ID NO: 107 | VL | DIVMTQAAPSVSVTPGESVSISCRSSKSLLHSSGNTYLYWFLQRPGQSPQLLIS |
| | | RMSNLASGVPDRFSGSGSGTAFTLRISRVEAEDVGVYYCMQHLEYPYTFGGG |
| | | TKLELK |
| SEQ ID NO: 108 | DNA VL | GATATTGTGATGACTCAGGCTGCACCCTCTGTATCTGTCACTCCTGGAGAG |
| | | TCAGTATCCATCTCCTGCAGGTCTAGTAAGAGTCTCCTCCATAGTAGTGGC |
| | | AACACTTACTTGTATTGGTTCCTGCAGAGGCCAGGCCAGTCTCCTCAGCTC |
| | | CTGATATCTCGGATGTCCAACCTTGCCTCAGGAGTCCCAGACAGGTTCAGT |
| | | GGCAGTGGGTCAGGAACTGCTTTCACACTGAGAATCAGTAGAGTGGAGG |
| | | CTGAGGATGTGGGTGTTTATTATTGTATGCAACATCTAGAATATCCGTACA |
| | | CGTTCGGAGGGGGGACCAAGCTGGAGCTAAAA |
| SEQ ID NO: 109 | Light Chain | DIVMTQAAPSVSVTPGESVSISCRSSKSLLHSSGNTYLYWFLQRPGQSPQLLIS |
| | | RMSNLASGVPDRFSGSGSGTAFTLRISRVEAEDVGVYYCMQHLEYPYTFGGG |
| | | TKLELKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTKS |
| | | FNRGEC |
| SEQ ID NO: 110 | DNA Light | GATATTGTGATGACTCAGGCTGCACCCTCTGTATCTGTCACTCCTGGAGAG |
| | Chain | TCAGTATCCATCTCCTGCAGGTCTAGTAAGAGTCTCCTCCATAGTAGTGGC |
| | | AACACTTACTTGTATTGGTTCCTGCAGAGGCCAGGCCAGTCTCCTCAGCTC |
| | | CTGATATCTCGGATGTCCAACCTTGCCTCAGGAGTCCCAGACAGGTTCAGT |
| | | GGCAGTGGGTCAGGAACTGCTTTCACACTGAGAATCAGTAGAGTGGAGG |
| | | CTGAGGATGTGGGTGTTTATTATTGTATGCAACATCTAGAATATCCGTACA |
| | | CGTTCGGAGGGGGGACCAAGCTGGAGCTAAAACGTACGGTGGCCGCTCC |
| | | CAGCGTGTTCATCTTCCCCCCCAGCGACGAGCAGCTGAAGAGTGGCACCG |
| | | CCAGCGTGGTGTGCCTGCTGAACAACTTCTACCCCCGGGAGGCCAAGGTG |
| | | CAGTGGAAGGTGGACAACGCCCTGCAGAGCGGCAACAGCCAGGAGAGC |
| | | GTCACCGAGCAGGACAGCAAGGACTCCACCTACAGCCTGAGCAGCACCCT |
| | | GACCCTGAGCAAGGCCGACTACGAGAAGCATAAGGTGTACGCCTGCGAG |
| | | GTGACCCACCAGGGCCTGTCCAGCCCCGTGACCAAGAGCTTCAACAGGGG |
| | | CGAGTGC |
| 960K03 | | |
| Parental | | |
| SEQ ID NO: 111 | HCDR1 (Combined) | GFSLSTGGMCVS |
| SEQ ID NO: 26 | HCDR2 (Combined) | LIDWDDDKYYSTSLKT |
| SEQ ID NO: 27 | HCDR3 (Combined) | AHSGSYFDF |
| SEQ ID NO: 112 | HCDR1 (Kabat) | TGGMCVS |
| SEQ ID NO: 26 | HCDR2 (Kabat) | LIDWDDDKYYSTSLKT |
| SEQ ID NO: 27 | HCDR3 (Kabat) | AHSGSYFDF |
| SEQ ID NO: 29 | HCDR1 (Chothia) | GFSLSTGGM |
| SEQ ID NO: 30 | HCDR2 (Chothia) | DWDDD |
| SEQ ID NO: 27 | HCDR3 (Chothia) | AHSGSYFDF |
| SEQ ID NO: 113 | HCDR1 (IMGT) | GFSLSTGGMC |
| SEQ ID NO: 32 | HCDR2 (IMGT) | IDWDDDK |
| SEQ ID NO: 33 | HCDR3 (IMGT) | ARAHSGSYFDF |
| SEQ ID NO: 114 | VH | QVTLRESGPALVKPTQTLTLTCTFSGFSLSTGGMCVSWIRQPPGKALEWLALI |
| | | DWDDDKYYSTSLKTRLTISKDTSKNQLVLTMTNMDPVDTATYYCARAHSGSY |
| | | FDFWGQGTLVTVSS |
| SEQ ID NO: 115 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTGGTGGAAT |
| | | GTGTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGCTGGTCCTTACAATGA |
| | | CCAACATGGACCCTGTGGACACAGCCACGTATTATTGTGCACGGGCTCATA |
| | | GTGGGAGCTACTTTGACTTCTGGGGCCAGGGAACCCTGGTCACCGTCTCC |
| | | TCA |
| SEQ ID NO: 116 | Heavy | QVTLRESGPALVKPTQTLTLTCTFSGFSLSTGGMCVSWIRQPPGKALEWLALI |
| | Chain | DWDDDKYYSTSLKTRLTISKDTSKNQLVLTMTNMDPVDTATYYCARAHSGSY |
| | | FDFWGQGTLVTVSSASTKGPSVFPLAPSSKSTSGGTAALGCLVKDYFPCPVTV |
| | | SWNSGALTSGVHTFPAVLQSSGLYSLSSVVIVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLIVLHQDWL |
| | | NGKEYKCKVSNKALAAPIEKTISKAKGQPREPQVYTLPPSRREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: 117 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACTGGTGGAAT |
| | | GTGTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGCTGGTCCTTACAATGA |
| | | CCAACATGGACCCTGTGGACACAGCCACGTATTATTGTGCACGGGCTCATA |
| | | GTGGGAGCTACTTTGACTTCTGGGGCCAGGGAACCCTGGTCACCGTCTCC |
| | | TCAGCCTCCACCAAGGGCCCATCGGTGTTTCCCCTGGCCCCCAGCAGCAAG |
| | | TCTACTTCCGGCGGAACTGCTGCCCTGGGTTGCCTGGTGAAGGACTACTTC |
| | | CCCTGTCCCGTGACAGTGTCCTGGAACTCTGGGGCTCTGACTTCCGGCGTG |
| | | CACACCTTCCCCGCCGTGCTGCAGAGCAGCGGCCTGTACAGCCTGAGCAG |
| | | CGTGGTGACAGTGCCCTCCAGCTCTCTGGGAACCCAGACCTATATCTGCAA |
| | | CGTGAACCACAAGCCCAGCAACACCAAGGTGGACAAGAGAGTGGAGCCC |
| | | AAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: 38 | LCDR1 (Combined) | RASQRISNWLA |
| SEQ ID NO: 39 | LCDR2 (Combined) | KASSLES |
| SEQ ID NO: 118 | LCDR3 (Combined) | QQFNSYWT |
| SEQ ID NO: 38 | LCDR1 (Kabat) | RASQRISNWLA |
| SEQ ID NO: 39 | LCDR2 (Kabat) | KASSLES |
| SEQ ID NO: 118 | LCDR3 (Kabat) | QQFNSYWT |
| SEQ ID NO: 41 | LCDR1 (Chothia) | SQRISNW |
| SEQ ID NO: 42 | LCDR2 (Chothia) | KAS |
| SEQ ID NO: 119 | LCDR3 (Chothia) | FNSYW |
| SEQ ID NO: 44 | LCDR1 (IMGT) | QRISNW |
| SEQ ID NO: 42 | LCDR2 (IMGT) | KAS |
| SEQ ID NO: 118 | LCDR3 (IMGT) | QQFNSYWT |
| SEQ ID NO: 120 | VL | DIQMTQSPSTLSASVGDRVTITCRASQRISNWLAWYQQKPGKAPKLLIYKASS |
| | | LESGVPSRFSGSGSGTEFTLTISSLQPDDFATYYCQQFNSYWTFGQGTKVEIK |
| SEQ ID NO: 121 | DNA VL | GACATCCAGATGACCCAGTCTCCTTCCACCCTGTCTGCATCTGTAGGAGAC |
| | | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAA |
| SEQ ID NO: 122 | Light Chain | DIQMTQSPSTLSASVGDRVTITCRASQRISNWLAWYQQKPGKAPKLLIYKASS |
| | | LESGVPSRFSGSGSGTEFTLTISSLQPDDFATYYCQQFNSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 123 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 958N05 | | |
| Parental | | |
| SEQ ID NO: 49 | HCDR1 (Combined) | GFSLSTSGISVS |
| SEQ ID NO: 26 | HCDR2 (Combined) | LIDWDDDKYYSTSLKT |
| SEQ ID NO: 50 | HCDR3 (Combined) | TPSGSYGRYFDL |
| SEQ ID NO: 51 | HCDR1 (Kabat) | TSGISVS |
| SEQ ID NO: 26 | HCDR2 (Kabat) | LIDWDDDKYYSTSLKT |
| SEQ ID NO: 50 | HCDR3 (Kabat) | TPSGSYGRYFDL |
| SEQ ID NO: 52 | HCDR1 (Chothia) | GFSLSTSGI |
| SEQ ID NO: 30 | HCDR2 (Chothia) | DWDDD |
| SEQ ID NO: 50 | HCDR3 (Chothia) | TPSGSYGRYFDL |
| SEQ ID NO: 53 | HCDR1 (IMGT) | GFSLSTSGIS |
| SEQ ID NO: 32 | HCDR2 (IMGT) | IDWDDDK |
| SEQ ID NO: 54 | HCDR3 (IMGT) | ARTPSGSYGRYFDL |
| SEQ ID NO: 55 | VH | QVTLRESGPALVKPTQTLTLTCTFSGFSLSTSGISVSWIRQPPGKALEWLALID |
| | | WDDDKYYSTSLKTRLTISKDTSKNQVVLTMTNMDPVDTATYYCARTPSGSYG |
| | | RYFDLWGRGTLVTVSS |
| SEQ ID NO: 56 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCA |
| SEQ ID NO: 57 | Heavy | QVTLRESGPALVKPTQTLTLTCTFSGFSLSTSGISVSWIRQPPGKALEWLALID |
| | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 58 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 59 | LCDR1 (Combined) | RASQSISNWLA |
| SEQ ID NO: 39 | LCDR2 (Combined) | KASSLES |
| SEQ ID NO: 124 | LCDR3 (Combined) | QQYNSYWT |
| SEQ ID NO: 59 | LCDR1 (Kabat) | RASQSISNWLA |
| SEQ ID NO: 39 | LCDR2 (Kabat) | KASSLES |
| SEQ ID NO: 124 | LCDR3 (Kabat) | QQYNSYWT |
| SEQ ID NO: 61 | LCDR1 (Chothia) | SQSISNW |
| SEQ ID NO: 42 | LCDR2 (Chothia) | KAS |
| SEQ ID NO: 125 | LCDR3 (Chothia) | YNSYW |
| SEQ ID NO: 63 | LCDR1 (IMGT) | QSISNW |
| SEQ ID NO: 42 | LCDR2 (IMGT) | KAS |
| SEQ ID NO: 124 | LCDR3 (IMGT) | QQYNSYWT |
| SEQ ID NO: 126 | VL | DIQLTQSPSTLSASVGDRVTITCRASQSISNWLAWYQQKPGKAPKLLIYKASSL |
| | | ESGVPSRFTGSGSGTEFTLTISSLQPDDFATYYCQQYNSYWTFGQGTKVEIK |
| SEQ ID NO: 127 | DNA VL | GACATCCAGTTGACCCAGTCTCCTTCCACCCTGTCTGCATCTGTAGGAGAC |
| | | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAA |
| SEQ ID NO: 128 | Light Chain | DIQLTQSPSTLSASVGDRVTITCRASQSISNWLAWYQQKPGKAPKLLIYKASSL |
| | | ESGVPSRFTGSGSGTEFTLTISSLQPDDFATYYCQQYNSYWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| SEQ ID NO: 129 | DNA Light | GACATCCAGTTGACCCAGTCTCCTTCCACCCTGTCTGCATCTGTAGGAGAC |
| | Chain | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 952P16 | | |
| Parental | | |
| SEQ ID NO: 74 | HCDR1 (Combined) | GFSLSTSGVSVS |
| SEQ ID NO: 26 | HCDR2 (Combined) | LIDWDDDKYYSTSLKT |
| SEQ ID NO: 50 | HCDR3 (Combined) | TPSGSYGRYFDL |
| SEQ ID NO: 75 | HCDR1 (Kabat) | TSGVSVS |
| SEQ ID NO: 26 | HCDR2 (Kabat) | LIDWDDDKYYSTSLKT |
| SEQ ID NO: 50 | HCDR3 (Kabat) | TPSGSYGRYFDL |
| SEQ ID NO: 76 | HCDR1 (Chothia) | GFSLSTSGV |
| SEQ ID NO: 30 | HCDR2 (Chothia) | DWDDD |
| SEQ ID NO: 50 | HCDR3 (Chothia) | TPSGSYGRYFDL |
| SEQ ID NO: 77 | HCDR1 (IMGT) | GFSLSTSGVS |
| SEQ ID NO: 32 | HCDR2 (IMGT) | IDWDDDK |
| SEQ ID NO: 54 | HCDR3 (IMGT) | ARTPSGSYGRYFDL |
| SEQ ID NO: 78 | VH | QVTLRESGPALVKPTQTLTLTCTFSGFSLSTSGVSVSWIRQPPGKALEWLALID |
| | | WDDDKYYSTSLKTRLTISKDTSKNQVVLTMTNMDPVDTATYYCARTPSGSYG |
| | | RYFDLWGRGTLVTVSS |
| SEQ ID NO: 79 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: 80 | Heavy | QVTLRESGPALVKPTQTLTLTCTFSGFSLSTSGVSVSWIRQPPGKALEWLALID |
| | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 81 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 59 | LCDR1 (Combined) | RASQSISNWLA |
| SEQ ID NO: 39 | LCDR2 (Combined) | KASSLES |
| SEQ ID NO: 124 | LCDR3 (Combined) | QQYNSYWT |
| SEQ ID NO: 59 | LCDR1 (Kabat) | RASQSISNWLA |
| SEQ ID NO: 39 | LCDR2 (Kabat) | KASSLES |
| SEQ ID NO: 124 | LCDR3 (Kabat) | QQYNSYWT |
| SEQ ID NO: 61 | LCDR1 (Chothia) | SQSISNW |
| SEQ ID NO: 42 | LCDR2 (Chothia) | KAS |
| SEQ ID NO: 125 | LCDR3 (Chothia) | YNSYW |
| SEQ ID NO: 63 | LCDR1 (IMGT) | QSISNW |
| SEQ ID NO: 42 | LCDR2 (IMGT) | KAS |
| SEQ ID NO: 124 | LCDR3 (IMGT) | QQYNSYWT |
| SEQ ID NO: 130 | VL | DIQMTQSPSTLSASVGDRVTITCRASQSISNWLAWYQQKPGKAPKLLIYKASS |
| | | LESGVPSRFSGSGSGTEFTLTISSLQPDDFATYYCQQYNSYWTFGQGTKVEIK |
| SEQ ID NO: 131 | DNA VL | GACATCCAGATGACCCAGTCTCCTTCCACCCTGTCTGCATCTGTAGGAGAC |
| | | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAA |
| SEQ ID NO: 132 | Light Chain | DIQMTQSPSTLSASVGDRVTITCRASQSISNWLAWYQQKPGKAPKLLIYKASS |
| | | LESGVPSRFSGSGSGTEFTLTISSLQPDDFATYYCQQYNSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 133 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 952G04 | | |
| Parental | | |
| SEQ ID NO: 88 | HCDR1 (Combined) | GFSLSTSGVSVT |
| SEQ ID NO: 26 | HCDR2 (Combined) | LIDWDDDKYYSTSLKT |
| SEQ ID NO: 50 | HCDR3 (Combined) | TPSGSYGRYFDL |
| SEQ ID NO: 89 | HCDR1 (Kabat) | TSGVSVT |
| SEQ ID NO: 26 | HCDR2 (Kabat) | LIDWDDDKYYSTSLKT |
| SEQ ID NO: 50 | HCDR3 (Kabat) | TPSGSYGRYFDL |
| SEQ ID NO: 76 | HCDR1 (Chothia) | GFSLSTSGV |
| SEQ ID NO: 30 | HCDR2 (Chothia) | DWDDD |
| SEQ ID NO: 50 | HCDR3 (Chothia) | TPSGSYGRYFDL |
| SEQ ID NO: 77 | HCDR1 (IMGT) | GFSLSTSGVS |
| SEQ ID NO: 32 | HCDR2 (IMGT) | IDWDDDK |
| SEQ ID NO: 54 | HCDR3 (IMGT) | ARTPSGSYGRYFDL |
| SEQ ID NO: 90 | VH | QVTLRESGPALVKPTQTLTLTCTFSGFSLSTSGVSVTWIRQPPGKALEWLALID |
| | | WDDDKYYSTSLKTRLAISKDTSKNQVVLTMTNMDPVDTATYYCARTPSGSYG |
| | | RYFDLWGRGTLVTVSS |
| SEQ ID NO: 91 | DNA VH | CAGGTCACCTTGAGGGAGTCTGGTCCTGCGCTGGTGAAACCCACACAGAC |
| | | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: 92 | Heavy | QVTLRESGPALVKPTQTLTLTCTFSGFSLSTSGVSVTWIRQPPGKALEWLALID |
| | Chain | WDDDKYYSTSLKTRLAISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 93 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 94 | LCDR1 (Combined) | RASQSISTWLA |
| SEQ ID NO: 95 | LCDR2 (Combined) | EASSLES |
| SEQ ID NO: 124 | LCDR3 (Combined) | QQYNSYWT |
| SEQ ID NO: 94 | LCDR1 (Kabat) | RASQSISTWLA |
| SEQ ID NO: 95 | LCDR2 (Kabat) | EASSLES |
| SEQ ID NO: 124 | LCDR3 (Kabat) | QQYNSYWT |
| SEQ ID NO: 96 | LCDR1 (Chothia) | SQSISTW |
| SEQ ID NO: 97 | LCDR2 (Chothia) | EAS |
| SEQ ID NO: 125 | LCDR3 (Chothia) | YNSYW |
| SEQ ID NO: 98 | LCDR1 (IMGT) | QSISTW |
| SEQ ID NO: 97 | LCDR2 (IMGT) | EAS |
| SEQ ID NO: 124 | LCDR3 (IMGT) | QQYNSYWT |
| SEQ ID NO: 134 | VL | DIQMTQSPSTLSASVGDRVTITCRASQSISTWLAWYQQKPGKAPKLLIYEASSL |
| | | ESGVPSRFSGSGSGTEFTLTISSLQPDDFATYYCQQYNSYWTFGQGTKVEIK |
| SEQ ID NO: 135 | DNA VL | GACATCCAGATGACCCAGTCTCCTTCCACCCTGTCTGCATCTGTAGGAGAC |
| | | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAA |
| SEQ ID NO: 136 | Light Chain | DIQMTQSPSTLSASVGDRVTITCRASQSISTWLAWYQQKPGKAPKLLIYEASSL |
| | | ESGVPSRFSGSGSGTEFTLTISSLQPDDFATYYCQQYNSYWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| SEQ ID NO: 137 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 892D15 | | |
| Parental | | |
| SEQ ID NO: 138 | HCDR1 (Combined) | GFSPSTSGMSVS |
| SEQ ID NO: 139 | HCDR2 (Combined) | LIDWDDDKYFSTSLKT |
| SEQ ID NO: 140 | HCDR3 (Combined) | AHSGSYFDY |
| SEQ ID NO: 141 | HCDR1 (Kabat) | TSGMSVS |
| SEQ ID NO: 139 | HCDR2 (Kabat) | LIDWDDDKYFSTSLKT |
| SEQ ID NO: 140 | HCDR3 (Kabat) | AHSGSYFDY |
| SEQ ID NO: 142 | HCDR1 (Chothia) | GFSPSTSGM |
| SEQ ID NO: 30 | HCDR2 (Chothia) | DWDDD |
| SEQ ID NO: 140 | HCDR3 (Chothia) | AHSGSYFDY |
| SEQ ID NO: 143 | HCDR1 (IMGT) | GFSPSTSGMS |
| SEQ ID NO: 32 | HCDR2 (IMGT) | IDWDDDK |
| SEQ ID NO: 144 | HCDR3 (IMGT) | ARAHSGSYFDY |
| SEQ ID NO: 145 | VH | QVTLRESGPALVKPTQTLTLTCTFSGFSPSTSGMSVSWIRQPPGKALEWLALID |
| | | WDDDKYFSTSLKTRLTISKDTSKNQVVLTMTNMDPVDTATYYCARAHSGSYF |
| | | DYWGQGTLVTVSS |
| SEQ ID NO: 146 | DNA VH | CAGGTCACCTTGAGGGAGTCTGGTCCTGCGCTGGTGAAACCCACACAGAC |
| | | CCTCACACTGACCTGCACCTTCTCTGGGTTCTCACCCAGCACTAGTGGAAT |
| | | GTCTGTGAGCTGGATCCGTCAGCCCCCAGGGAAGGCCCTGGAGTGGCTTG |
| | | CACTTATTGATTGGGATGATGATAAATACTTTAGCACATCTCTGAAGACCA |
| | | GGCTCACCATCTCCAAGGACACCTCCAAAAACCAGGTGGTCCTTACAATGA |
| | | CCAACATGGACCCTGTAGACACAGCCACGTATTATTGTGCACGGGCCCATA |
| | | GTGGGAGCTACTTTGACTACTGGGGCCAGGGAACCCTGGTCACCGTCTCC |
| | | TCA |
| SEQ ID NO: 147 | Heavy | QVTLRESGPALVKPTQTLTLTCTFSGFSPSTSGMSVSWIRQPPGKALEWLALID |
| | Chain | WDDDKYFSTSLKTRLTISKDTSKNQVVLTMTNMDPVDTATYYCARAHSGSYF |
| | | DYWGQGTLVTVSSASTKGPSVFPLAPSSKSTSGGTAALGCLVKDYFPCPVTVS |
| | | WNSGALTSGVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISRTPEVTCVVV |
| | | AVSHEDPEVKFNWYVDGVEVHNAKTKPREEQYNSTYRVVSVLTVLHQDWLN |
| | | GKEYKCKVSNKALAAPIEKTISKAKGQPREPQVYTLPPSREEMTKNQVSLTCLV |
| | | KGFYPCDIAVEWESNGQPENNYKTTPPVLDSDGSFFLYSKLTVDKSRWQQGN |
| | | VFSCSVMHEALHNHYTQKSLSLSPGK |
| SEQ ID NO: 148 | DNA Heavy | CAGGTCACCTTGAGGGAGTCTGGTCCTGCGCTGGTGAAACCCACACAGAC |
| | Chain | CCTCACACTGACCTGCACCTTCTCTGGGTTCTCACCCAGCACTAGTGGAAT |
| | | GTCTGTGAGCTGGATCCGTCAGCCCCCAGGGAAGGCCCTGGAGTGGCTTG |
| | | CACTTATTGATTGGGATGATGATAAATACTTTAGCACATCTCTGAAGACCA |
| | | GGCTCACCATCTCCAAGGACACCTCCAAAAACCAGGTGGTCCTTACAATGA |
| | | CCAACATGGACCCTGTAGACACAGCCACGTATTATTGTGCACGGGCCCATA |
| | | GTGGGAGCTACTTTGACTACTGGGGCCAGGGAACCCTGGTCACCGTCTCC |
| | | TCAGCCTCCACCAAGGGCCCATCGGTGTTTCCCCTGGCCCCCAGCAGCAAG |
| | | TCTACTTCCGGCGGAACTGCTGCCCTGGGTTGCCTGGTGAAGGACTACTTC |
| | | CCCTGTCCCGTGACAGTGTCCTGGAACTCTGGGGCTCTGACTTCCGGCGTG |
| | | CACACCTTCCCCGCCGTGCTGCAGAGCAGCGGCCTGTACAGCCTGAGCAG |
| | | CGTGGTGACAGTGCCCTCCAGCTCTCTGGGAACCCAGACCTATATCTGCAA |
| | | CGTGAACCACAAGCCCAGCAACACCAAGGTGGACAAGAGAGTGGAGCCC |
| | | AAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: 59 | LCDR1 (Combined) | RASQSISNWLA |
| SEQ ID NO: 39 | LCDR2 (Combined) | KASSLES |
| SEQ ID NO: 118 | LCDR3 (Combined) | QQFNSYWT |
| SEQ ID NO: 59 | LCDR1 (Kabat) | RASQSISNWLA |
| SEQ ID NO: 39 | LCDR2 (Kabat) | KASSLES |
| SEQ ID NO: 118 | LCDR3 (Kabat) | QQFNSYWT |
| SEQ ID NO: 61 | LCDR1 (Chothia) | SQSISNW |
| SEQ ID NO: 42 | LCDR2 (Chothia) | KAS |
| SEQ ID NO: 119 | LCDR3 (Chothia) | FNSYW |
| SEQ ID NO: 63 | LCDR1 (IMGT) | QSISNW |
| SEQ ID NO: 42 | LCDR2 (IMGT) | KAS |
| SEQ ID NO: 118 | LCDR3 (IMGT) | QQFNSYWT |
| SEQ ID NO: 149 | VL | DIQMTQSPSTLSASVGDRVTITCRASQSISNWLAWYQQKPGKAPKLLIYKASS |
| | | LESGVPSRFSGSGSGTEFTLTISSLQPDDFATYYCQQFNSYWTFGQGTKVEIK |
| SEQ ID NO: 150 | DNA VL | GACATCCAGATGACCCAGTCTCCTTCCACCCTGTCTGCATCTGTAGGAGAC |
| | | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAA |
| SEQ ID NO: 151 | Light Chain | DIQMTQSPSTLSASVGDRVTITCRASQSISNWLAWYQQKPGKAPKLLIYKASS |
| | | LESGVPSRFSGSGSGTEFTLTISSLQPDDFATYYCQQFNSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFRGEC |
| SEQ ID NO: 152 | DNA Light | GACATCCAGATGACCCAGTCTCCTTCCACCCTGTCTGCATCTGTAGGAGAC |
| | Chain | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |
| 914M09 | | |
| Parental | | |
| SEQ ID NO: 153 | HCDR1 (Combined) | GGSISSYYWN |
| SEQ ID NO: 154 | HCDR2 (Combined) | RIYTSGSTNYNPSLKS |
| SEQ ID NO: 155 | HCDR3 (Combined) | DSGGFYYYYGMDV |
| SEQ ID NO: 156 | HCDR1 (Kabat) | SYYWN |
| SEQ ID NO: 154 | HCDR2 (Kabat) | RIYTSGSTNYNPSLKS |
| SEQ ID NO: 155 | HCDR3 (Kabat) | DSGGFYYYYGMDV |
| SEQ ID NO: 157 | HCDR1 (Chothia) | GGSISSY |
| SEQ ID NO: 158 | HCDR2 (Chothia) | YTSGS |
| SEQ ID NO: 155 | HCDR3 (Chothia) | DSGGFYYYYGMDV |
| SEQ ID NO: 159 | HCDR1 (IMGT) | GGSISSYY |
| SEQ ID NO: 160 | HCDR2 (IMGT) | IYTSGST |
| SEQ ID NO: 161 | HCDR3 (IMGT) | ARDSGGFYYYYGMDV |
| SEQ ID NO: 162 | VH | QVQLQESGPGLVKPSETLSLTCAVSGGSISSYYWNLIRQPAGKGLEWIGRIYTS |
| | | GSTNYNPSLKSRVTMSVDTSKNQFSLKLSSVTAADTAVYYCARDSGGFYYYYG |
| | | MDVWGQGTTVTVSS |
| SEQ ID NO: 163 | DNA VH | CAGGTGCAGCTGCAGGAGTCGGGCCCAGGACTGGTGAAGCCTTCGGAGA |
| | | CCCTGTCCCTCACCTGCGCTGTCTCTGGTGGCTCCATCAGTAGTTACTACTG |
| | | GAACTTAATCCGGCAGCCCGCCGGGAAGGGACTGGAGTGGATTGGGCGT |
| | | ATCTATACCAGTGGGAGCACCAACTACAACCCCTCCCTCAAGAGTCGAGTC |
| | | ACCATGTCAGTAGACACGTCCAAGAACCAGTTCTCCCTGAAGCTGAGCTCT |
| | | GTGACCGCCGCGGACACGGCCGTGTATTACTGTGCGAGAGACTCCGGGG |
| | | GGTTCTACTACTACTACGGTATGGACGTCTGGGGCCAAGGGACCACGGTC |
| | | ACCGTCTCCTCA |
| SEQ ID NO: 164 | Heavy | QVQLQESGPGLVKPSETLSLTCAVSGGSISSYYWNLIRQPAGKGLEWIGRIYTS |
| | Chain | GSTNYNPSLKSRVTMSVDTSKNQFSLKLSSVTAADTAVYYCARDSGGFYYYYG |
| | | MDVWGQGTTVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 165 | DNA Heavy | CAGGTGCAGCTGCAGGAGTCGGGCCCAGGACTGGTGAAGCCTTCGGAGA |
| | Chain | CCCTGTCCCTCACCTGCGCTGTCTCTGGTGGCTCCATCAGTAGTTACTACTG |
| | | GAACTTAATCCGGCAGCCCGCCGGGAAGGGACTGGAGTGGATTGGGCGT |
| | | ATCTATACCAGTGGGAGCACCAACTACAACCCCTCCCTCAAGAGTCGAGTC |
| | | ACCATGTCAGTAGACACGTCCAAGAACCAGTTCTCCCTGAAGCTGAGCTCT |
| | | GTGACCGCCGCGGACACGGCCGTGTATTACTGTGCGAGAGACTCCGGGG |
| | | GGTTCTACTACTACTACGGTATGGACGTCTGGGGCCAAGGGACCACGGTC |
| | | ACCGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 166 | LCDR1 (Combined) | RASQGISSYLA |
| SEQ ID NO: 167 | LCDR2 (Combined) | AASTLQG |
| SEQ ID NO: 168 | LCDR3 (Combined) | QQLNSYPWT |
| SEQ ID NO: 166 | LCDR1 (Kabat) | RASQGISSYLA |
| SEQ ID NO: 167 | LCDR2 (Kabat) | AASTLQG |
| SEQ ID NO: 168 | LCDR3 (Kabat) | QQLNSYPWT |
| SEQ ID NO: 169 | LCDR1 (Chothia) | SQGISSY |
| SEQ ID NO: 170 | LCDR2 (Chothia) | AAS |
| SEQ ID NO: 171 | LCDR3 (Chothia) | LNSYPW |
| SEQ ID NO: 172 | LCDR1 (IMGT) | QGISSY |
| SEQ ID NO: 173 | LCDR2 (IMGT) | AASTLQGGVP |
| SEQ ID NO: 168 | LCDR3 (IMGT) | QQLNSYPWT |
| SEQ ID NO: 174 | VL | DIQLTQSPSFLSASVGDRVTITCRASQGISSYLAWYQQKPGKAPKLLIYAASTLQ |
| | | GGVPSRFSGSGSGTEFTLTISSLQPEDFATYHCQQLNSYPWTFGQGTKVEIK |
| SEQ ID NO: 175 | DNA VL | GACATCCAGTTGACCCAGTCTCCATCCTTCCTGTCTGCATCTGTAGGAGAC |
| | | AGAGTCACCATCACTTGCCGGGCCAGTCAGGGCATTAGCAGTTATTTAGCC |
| | | TGGTATCAGCAAAAACCAGGGAAAGCCCCTAAGCTCCTGATCTATGCTGC |
| | | ATCCACCTTGCAAGGTGGGGTCCCATCAAGGTTCAGCGGCAGTGGATCTG |
| | | GGACAGAATTCACTCTCACAATCAGCAGCCTGCAGCCTGAAGATTTTGCAA |
| | | CTTATCACTGTCAACAGCTTAATAGTTACCCGTGGACGTTCGGCCAAGGGA |
| | | CCAAGGTGGAAATCAAA |
| SEQ ID NO: 176 | Light Chain | DIQLTQSPSFLSASVGDRVTITCRASQGISSYLAWYQQKPGKAPKLLIYAASTLQ |
| | | GGVPSRFSGSGSGTEFTLTISSLQPEDFATYHCQQLNSYPWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| SEQ ID NO: 177 | DNA Light | GACATCCAGTTGACCCAGTCTCCATCCTTCCTGTCTGCATCTGTAGGAGAC |
| | Chain | AGAGTCACCATCACTTGCCGGGCCAGTCAGGGCATTAGCAGTTATTTAGCC |
| | | TGGTATCAGCAAAAACCAGGGAAAGCCCCTAAGCTCCTGATCTATGCTGC |
| | | ATCCACCTTGCAAGGTGGGGTCCCATCAAGGTTCAGCGGCAGTGGATCTG |
| | | GGACAGAATTCACTCTCACAATCAGCAGCCTGCAGCCTGAAGATTTTGCAA |
| | | CTTATCACTGTCAACAGCTTAATAGTTACCCGTGGACGTTCGGCCAAGGGA |
| | | CCAAGGTGGAAATCAAACGAACTGTGGCTGCACCAAGCGTGTTCATCTTC |
| | | CCCCCCAGCGACGAGCAGCTGAAGAGTGGCACCGCCAGCGTGGTGTGCCT |
| | | GCTGAACAACTTCTACCCCCGGGAGGCCAAGGTGCAGTGGAAGGTGGAC |
| | | AACGCCCTGCAGAGCGGCAACAGCCAGGAGAGCGTCACCGAGCAGGACA |
| | | GCAAGGACTCCACCTACAGCCTGAGCAGCACCCTGACCCTGAGCAAGGCC |
| | | GACTACGAGAAGCATAAGGTGTACGCCTGCGAGGTGACCCACCAGGGCC |
| | | TGTCCAGCCCCGTGACCAAGAGCTTCAACAGGGGCGAGTGC |
| 906C18 | | |
| Parental | | |
| SEQ ID NO: 178 | HCDR1 (Combined) | GFTFNNYWMN |
| SEQ ID NO: 179 | HCDR2 (Combined) | NIRQDGSEKYYVDSVKG |
| SEQ ID NO: 180 | HCDR3 (Combined) | ERAYCSTTSCPDYSNDY |
| SEQ ID NO: 181 | HCDR1 (Kabat) | NYWMN |
| SEQ ID NO: 179 | HCDR2 (Kabat) | NIRQDGSEKYYVDSVKG |
| SEQ ID NO: 180 | HCDR3 (Kabat) | ERAYCSTTSCPDYSNDY |
| SEQ ID NO: 182 | HCDR1 (Chothia) | GFTFNNY |
| SEQ ID NO: 183 | HCDR2 (Chothia) | RQDGSE |
| SEQ ID NO: 180 | HCDR3 (Chothia) | ERAYCSTTSCPDYSNDY |
| SEQ ID NO: 184 | HCDR1 (IMGT) | GFTFNNYW |
| SEQ ID NO: 185 | HCDR2 (IMGT) | IRQDGSEK |
| SEQ ID NO: 186 | HCDR3 (IMGT) | ARERAYCSTTSCPDYSNDY |
| SEQ ID NO: 187 | VH | EVQLVESGGGLVQPGGSLRLSCAASGFTFNNYWMNWVRQAPGKGLEWVA |
| | | NIRQDGSEKYYVDSVKGRFTISRDNAKNSLFLQMNSLRAEDTAVYYCARERAY |
| | | CSTTSCPDYSNDYWGQGTLVTVSS |
| SEQ ID NO: 188 | DNA VH | GAGGTGCAGCTGGTGGAGTCTGGGGGAGGCTTGGTCCAGCCTGGGGGGT |
| | | CCCTGAGGCTCTCCTGTGCAGCCTCTGGATTCACCTTTAATAACTATTGGAT |
| | | GAACTGGGTCCGCCAGGCTCCAGGGAAGGGGCTGGAGTGGGTGGCCAAC |
| | | ATAAGACAAGATGGAAGTGAAAAATACTATGTGGACTCTGTGAAGGGCC |
| | | GATTCACCATCTCCAGAGACAACGCCAAGAACTCACTGTTTCTACAAATGA |
| | | ACAGCCTGAGAGCCGAGGACACGGCTGTATATTACTGTGCGAGAGAGAG |
| | | GGCCTATTGTAGTACTACCAGCTGCCCTGACTACAGTAATGACTACTGGGG |
| | | CCAGGGAACCCTGGTCACCGTCTCCTCA |
| SEQ ID NO: 189 | Heavy | EVQLVESGGGLVQPGGSLRLSCAASGFTFNNYWMNWVRQAPGKGLEWVA |
| | Chain | NIRQDGSEKYYVDSVKGRFTISRDNAKNSLFLQMNSLRAEDTAVYYCARERAY |
| | | CSTTSCPDYSNDYWGQGTLVTVSSASTKGPSVFPLAPSSKSTSGGTAALGCLV |
| | | KDYFPCPVIVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVAVSHEDPEVKFNWYVDGVEVHNAKTKPREEQYNSTYRVVS |
| | | VLTVLHQDWLNGKEYKCKVSNKALAAPIEKTISKAKGQPREPQVYTLPPSREE |
| | | MTKNQVSLTCLVKGFYPCDIAVEWESNGQPENNYKTTPPVLDSDGSFFLYSKL |
| | | TVDKSRWQQGNVFSCSVMHEALHNHYTQKSLSLSPGK |
| SEQ ID NO: 190 | DNA Heavy | GAGGTGCAGCTGGTGGAGTCTGGGGGAGGCTTGGTCCAGCCTGGGGGGT |
| | Chain | CCCTGAGGCTCTCCTGTGCAGCCTCTGGATTCACCTTTAATAACTATTGGAT |
| | | GAACTGGGTCCGCCAGGCTCCAGGGAAGGGGCTGGAGTGGGTGGCCAAC |
| | | ATAAGACAAGATGGAAGTGAAAAATACTATGTGGACTCTGTGAAGGGCC |
| | | GATTCACCATCTCCAGAGACAACGCCAAGAACTCACTGTTTCTACAAATGA |
| | | ACAGCCTGAGAGCCGAGGACACGGCTGTATATTACTGTGCGAGAGAGAG |
| | | GGCCTATTGTAGTACTACCAGCTGCCCTGACTACAGTAATGACTACTGGGG |
| | | CCAGGGAACCCTGGTCACCGTCTCCTCAGCCTCCACCAAGGGCCCATCGGT |
| | | GTTTCCCCTGGCCCCCAGCAGCAAGTCTACTTCCGGCGGAACTGCTGCCCT |
| | | GGGTTGCCTGGTGAAGGACTACTTCCCCTGTCCCGTGACAGTGTCCTGGA |
| | | ACTCTGGGGCTCTGACTTCCGGCGTGCACACCTTCCCCGCCGTGCTGCAGA |
| | | GCAGCGGCCTGTACAGCCTGAGCAGCGTGGTGACAGTGCCCTCCAGCTCT |
| | | CTGGGAACCCAGACCTATATCTGCAACGTGAACCACAAGCCCAGCAACAC |
| | | CAAGGTGGACAAGAGAGTGGAGCCCAAGAGCTGCGACAAGACCCACACC |
| | | TGCCCCCCCTGCCCAGCTCCAGAACTGCTGGGAGGGCCTTCCGTGTTCCTG |
| | | TTCCCCCCCAAGCCCAAGGACACCCTGATGATCAGCAGGACCCCCGAGGT |
| | | GACCTGCGTGGTGGTGGCCGTGTCCCACGAGGACCCAGAGGTGAAGTTC |
| | | AACTGGTACGTGGACGGCGTGGAGGTGCACAACGCCAAGACCAAGCCCA |
| | | GAGAGGAGCAGTACAACAGCACCTACAGGGTGGTGTCCGTGCTGACCGT |
| | | GCTGCACCAGGACTGGCTGAACGGCAAAGAATACAAGTGCAAAGTCTCCA |
| | | ACAAGGCCCTGGCTGCCCCAATCGAAAAGACAATCAGCAAGGCCAAGGG |
| | | CCAGCCACGGGAGCCCCAGGTGTACACCCTGCCCCCCAGCCGGGAGGAG |
| | | ATGACCAAGAACCAGGTGTCCCTGACCTGTCTGGTGAAGGGCTTCTACCCC |
| | | TGTGATATCGCCGTGGAGTGGGAGAGCAACGGCCAGCCCGAGAACAACT |
| | | ACAAGACCACCCCCCCAGTGCTGGACAGCGACGGCAGCTTCTTCCTGTACA |
| | | GCAAGCTGACCGTGGACAAGTCCAGGTGGCAGCAGGGCAACGTGTTCAG |
| | | CTGCAGCGTGATGCACGAGGCCCTGCACAACCACTACACCCAGAAGTCCC |
| | | TGAGCCTGAGCCCCGGCAAG |
| SEQ ID NO: 191 | LCDR1 (Combined) | RASQTINNNLA |
| SEQ ID NO: 192 | LCDR2 (Combined) | GASTGAT |
| SEQ ID NO: 193 | LCDR3 (Combined) | QQYNNWPRGLT |
| SEQ ID NO: 191 | LCDR1 (Kabat) | RASQTINNNLA |
| SEQ ID NO: 192 | LCDR2 (Kabat) | GASTGAT |
| SEQ ID NO: 193 | LCDR3 (Kabat) | QQYNNWPRGLT |
| SEQ ID NO: 194 | LCDR1 (Chothia) | SQTINNN |
| SEQ ID NO: 195 | LCDR2 (Chothia) | GAS |
| SEQ ID NO: 196 | LCDR3 (Chothia) | YNNWPRGL |
| SEQ ID NO: 197 | LCDR1 (IMGT) | QTINNN |
| SEQ ID NO: 198 | LCDR2 (IMGT) | GASTGATGIP |
| SEQ ID NO: 193 | LCDR3 (IMGT) | QQYNNWPRGLT |
| SEQ ID NO: 199 | VL | EIVMTQSPATLSVSPGERATLSCRASQTINNNLAWFQQKPGQTPRLLIYGAST |
| | | GATGIPARFSGSGSGTEFTLTISSLQSEDFAVYYCQQYNNWPRGLTFGGGTKV |
| | | EIK |
| SEQ ID NO: 200 | DNA VL | GAAATAGTGATGACGCAGTCTCCAGCCACCCTGTCTGTGTCTCCAGGGGA |
| | | AAGAGCCACCCTCTCCTGCAGGGCCAGTCAGACTATTAACAACAACTTAGC |
| | | CTGGTTCCAGCAGAAACCTGGCCAGACTCCCAGGCTCCTCATCTATGGTGC |
| | | ATCCACCGGGGCCACTGGTATCCCAGCCAGGTTCAGTGGCAGTGGGTCTG |
| | | GGACAGAGTTCACTCTCACCATCAGCAGCCTGCAGTCTGAAGATTTTGCAG |
| | | TTTATTACTGTCAGCAGTATAATAACTGGCCTCGAGGACTCACTTTCGGCG |
| | | GAGGGACCAAGGTGGAGATCAAA |
| SEQ ID NO: 201 | Light Chain | EIVMTQSPATLSVSPGERATLSCRASQTINNNLAWFQQKPGQTPRLLIYGAST |
| | | GATGIPARFSGSGSGTEFTLTISSLQSEDFAVYYCQQYNNWPRGLTFGGGTKV |
| | | EIKRTVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTKSFNR |
| | | GEC |
| SEQ ID NO: 202 | DNA Light | GAAATAGTGATGACGCAGTCTCCAGCCACCCTGTCTGTGTCTCCAGGGGA |
| | Chain | AAGAGCCACCCTCTCCTGCAGGGCCAGTCAGACTATTAACAACAACTTAGC |
| | | CTGGTTCCAGCAGAAACCTGGCCAGACTCCCAGGCTCCTCATCTATGGTGC |
| | | ATCCACCGGGGCCACTGGTATCCCAGCCAGGTTCAGTGGCAGTGGGTCTG |
| | | GGACAGAGTTCACTCTCACCATCAGCAGCCTGCAGTCTGAAGATTTTGCAG |
| | | TTTATTACTGTCAGCAGTATAATAACTGGCCTCGAGGACTCACTTTCGGCG |
| | | GAGGGACCAAGGTGGAGATCAAACGAACTGTGGCTGCACCAAGCGTGTT |
| | | CATCTTCCCCCCCAGCGACGAGCAGCTGAAGAGTGGCACCGCCAGCGTGG |
| | | TGTGCCTGCTGAACAACTTCTACCCCCGGGAGGCCAAGGTGCAGTGGAAG |
| | | GTGGACAACGCCCTGCAGAGCGGCAACAGCCAGGAGAGCGTCACCGAGC |
| | | AGGACAGCAAGGACTCCACCTACAGCCTGAGCAGCACCCTGACCCTGAGC |
| | | AAGGCCGACTACGAGAAGCATAAGGTGTACGCCTGCGAGGTGACCCACC |
| | | AGGGCCTGTCCAGCCCCGTGACCAAGAGCTTCAACAGGGGCGAGTGC |
| 956E02 | | |
| Parental | | |
| SEQ ID NO: 203 | HCDR1 (Combined) | GYTFTGYYIN |
| SEQ ID NO: 204 | HCDR2 (Combined) | WINPNSGDTNYAQKFQG |
| SEQ ID NO: 205 | HCDR3 (Combined) | ENSGYGKLFDY |
| SEQ ID NO: 206 | HCDR1 (Kabat) | GYYIN |
| SEQ ID NO: 204 | HCDR2 (Kabat) | WINPNSGDTNYAQKFQG |
| SEQ ID NO: 205 | HCDR3 (Kabat) | ENSGYGKLFDY |
| SEQ ID NO: 207 | HCDR1 (Chothia) | GYTFTGY |
| SEQ ID NO: 208 | HCDR2 (Chothia) | NPNSGD |
| SEQ ID NO: 205 | HCDR3 (Chothia) | ENSGYGKLFDY |
| SEQ ID NO: 209 | HCDR1 (IMGT) | GYTFTGYY |
| SEQ ID NO: 210 | HCDR2 (IMGT) | INPNSGDT |
| SEQ ID NO: 211 | HCDR3 (IMGT) | ARENSGYGKLFDY |
| SEQ ID NO: 212 | VH | QVQLVQSGAEVKKPGASVKVSCKASGYTFTGYYINWVRQAPGQGLEWMG |
| | | WINPNSGDTNYAQKFQGRVTMTRDPSISTAYMELSRLRSDDTAVYYCARENS |
| | | GYGKLFDYWGQGTLVTVSS |
| SEQ ID NO: 213 | DNA VH | CAGGTGCAGCTGGTGCAGTCTGGGGCTGAGGTGAAGAAGCCTGGGGCCT |
| | | CAGTGAAGGTCTCCTGCAAGGCTTCTGGATACACCTTCACCGGCTACTATA |
| | | TAAATTGGGTGCGACAGGCCCCTGGACAAGGGCTTGAGTGGATGGGATG |
| | | GATCAACCCTAACAGTGGTGACACAAACTATGCACAGAAGTTTCAGGGCA |
| | | GGGTCACCATGACCAGGGACCCGTCCATCAGCACAGCCTACATGGAGCTG |
| | | AGCAGGCTGAGATCTGACGACACGGCCGTGTATTACTGTGCGAGAGAGA |
| | | ATAGTGGCTACGGGAAGCTTTTTGACTACTGGGGCCAGGGAACCCTGGTC |
| | | ACCGTCTCCTCA |
| SEQ ID NO: 214 | Heavy | QVQLVQSGAEVKKPGASVKVSCKASGYTFTGYYINWVRQAPGQGLEWMG |
| | Chain | WINPNSGDTNYAQKFQGRVTMTRDPSISTAYMELSRLRSDDTAVYYCARENS |
| | | GYGKLFDYWGQGTLVTVSSASTKGPSVFPLAPSSKSTSGGTAALGCLVKDYFP |
| | | CPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPE |
| | | VTCVVVAVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSREEMTKN |
| | | QVSLTCLVKGFYPCDIAVEWESNGQPENNYKTTPPVLDSDGSFFLYSKLTVDK |
| | | SRWQQGNVFSCSVMHEALHNHYTQKSLSLSPGK |
| SEQ ID NO: 215 | DNA Heavy | CAGGTGCAGCTGGTGCAGTCTGGGGCTGAGGTGAAGAAGCCTGGGGCCT |
| | Chain | CAGTGAAGGTCTCCTGCAAGGCTTCTGGATACACCTTCACCGGCTACTATA |
| | | TAAATTGGGTGCGACAGGCCCCTGGACAAGGGCTTGAGTGGATGGGATG |
| | | GATCAACCCTAACAGTGGTGACACAAACTATGCACAGAAGTTTCAGGGCA |
| | | GGGTCACCATGACCAGGGACCCGTCCATCAGCACAGCCTACATGGAGCTG |
| | | AGCAGGCTGAGATCTGACGACACGGCCGTGTATTACTGTGCGAGAGAGA |
| | | ATAGTGGCTACGGGAAGCTTTTTGACTACTGGGGCCAGGGAACCCTGGTC |
| | | ACCGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: 216 | LCDR1 (Combined) | RSSQSLLHSNGYNYLD |
| SEQ ID NO: 217 | LCDR2 (Combined) | LGSNRAS |
| SEQ ID NO: 218 | LCDR3 (Combined) | MQALQTPYT |
| SEQ ID NO: 216 | LCDR1 (Kabat) | RSSQSLLHSNGYNYLD |
| SEQ ID NO: 217 | LCDR2 (Kabat) | LGSNRAS |
| SEQ ID NO: 218 | LCDR3 (Kabat) | MQALQTPYT |
| SEQ ID NO: 219 | LCDR1 (Chothia) | SQSLLHSNGYNY |
| SEQ ID NO: 220 | LCDR2 (Chothia) | LGS |
| SEQ ID NO: 221 | LCDR3 (Chothia) | ALQTPY |
| SEQ ID NO: 222 | LCDR1 (IMGT) | QSLLHSNGYNY |
| SEQ ID NO: 220 | LCDR2 (IMGT) | LGS |
| SEQ ID NO: 218 | LCDR3 (IMGT) | MQALQTPYT |
| SEQ ID NO: 223 | VL | DIVMTQSPLSLPGTPGEPASISCRSSQSLLHSNGYNYLDWYLQKPGQSPQFLIY |
| | | LGSNRASGVPDRFSGSGSGTDFTLKISRVEAEDVGVYYCMQALQTPYTFGQG |
| | | TKLEIK |
| SEQ ID NO: 224 | DNA VL | GATATTGTGATGACTCAGTCTCCACTCTCCCTGCCCGGCACCCCTGGAGAG |
| | | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGTTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATGCAAGCTCTACAAACTCCGTACA |
| | | CTTTTGGCCAGGGGACCAAGCTGGAGATCAAA |
| SEQ ID NO: 225 | Light Chain | DIVMTQSPLSLPGTPGEPASISCRSSQSLLHSNGYNYLDWYLQKPGQSPQFLIY |
| | | LGSNRASGVPDRFSGSGSGTDFTLKISRVEAEDVGVYYCMQALQTPYTFGQG |
| | | TKLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTKS |
| | | FNRGEC |
| SEQ ID NO: 226 | DNA Light | GATATTGTGATGACTCAGTCTCCACTCTCCCTGCCCGGCACCCCTGGAGAG |
| | Chain | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGTTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATGCAAGCTCTACAAACTCCGTACA |
| | | CTTTTGGCCAGGGGACCAAGCTGGAGATCAAACGAACTGTGGCTGCACCA |
| | | AGCGTGTTCATCTTCCCCCCCAGCGACGAGCAGCTGAAGAGTGGCACCGC |
| | | CAGCGTGGTGTGCCTGCTGAACAACTTCTACCCCCGGGAGGCCAAGGTGC |
| | | AGTGGAAGGTGGACAACGCCCTGCAGAGCGGCAACAGCCAGGAGAGCGT |
| | | CACCGAGCAGGACAGCAAGGACTCCACCTACAGCCTGAGCAGCACCCTGA |
| | | CCCTGAGCAAGGCCGACTACGAGAAGCATAAGGTGTACGCCTGCGAGGT |
| | | GACCCACCAGGGCCTGTCCAGCCCCGTGACCAAGAGCTTCAACAGGGGCG |
| | | AGTGC |
| 942K11 | | |
| Parental | | |
| SEQ ID NO: 227 | HCDR1 (Combined) | GGSISSYYWT |
| SEQ ID NO: 228 | HCDR2 (Combined) | RVFTSESTNYNPSLKN |
| SEQ ID NO: 229 | HCDR3 (Combined) | DRGTYLGGFDP |
| SEQ ID NO: 230 | HCDR1 (Kabat) | SYYWT |
| SEQ ID NO: 228 | HCDR2 (Kabat) | RVFTSESTNYNPSLKN |
| SEQ ID NO: 229 | HCDR3 (Kabat) | DRGTYLGGFDP |
| SEQ ID NO: 157 | HCDR1 (Chothia) | GGSISSY |
| SEQ ID NO: 231 | HCDR2 (Chothia) | FTSES |
| SEQ ID NO: 229 | HCDR3 (Chothia) | DRGTYLGGFDP |
| SEQ ID NO: 159 | HCDR1 (IMGT) | GGSISSYY |
| SEQ ID NO: 232 | HCDR2 (IMGT) | VFTSEST |
| SEQ ID NO: 233 | HCDR3 (IMGT) | ARDRGTYLGGFDP |
| SEQ ID NO: 234 | VH | QVQLQESGPGLVKPSETLSLTCTVSGGSISSYYWTWIRQPAGKGLEWIGRVFT |
| | | SESTNYNPSLKNRVTMSVDTSKNQFSLRLNSVTAADTAMYYCARDRGTYLGG |
| | | FDPWGQGTLVTVSS |
| SEQ ID NO: 235 | DNA VH | CAGGTGCAGCTGCAGGAGTCGGGCCCAGGACTGGTGAAGCCTTCGGAGA |
| | | CCCTGTCCCTCACCTGCACTGTCTCTGGTGGCTCCATCAGTAGTTACTACTG |
| | | GACCTGGATCCGGCAGCCCGCCGGGAAGGGACTGGAGTGGATTGGGCGT |
| | | GTCTTTACCAGTGAGAGCACCAACTACAACCCCTCCCTCAAGAATCGAGTC |
| | | ACCATGTCAGTAGACACGTCCAAGAACCAGTTCTCCCTGAGGCTGAATTCT |
| | | GTGACCGCCGCGGACACGGCCATGTATTACTGTGCGAGAGACCGGGGGA |
| | | CCTACCTAGGGGGGTTCGACCCCTGGGGCCAGGGAACCCTGGTCACCGTC |
| | | TCCTCA |
| SEQ ID NO: 236 | Heavy | QVQLQESGPGLVKPSETLSLTCTVSGGSISSYYWTWIRQPAGKGLEWIGRVFT |
| | Chain | SESTNYNPSLKNRVTMSVDTSKNQFSLRLNSVTAADTAMYYCARDRGTYLGG |
| | | FDPWGQGTLVTVSSASTKGPSVFPLAPSSKSTSGGTAALGCLVKDYFPCPVTV |
| | | SWNSGALTSGVHTFPAVLQSSGLYSLSSVVIVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLIVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: 237 | DNA Heavy | CAGGTGCAGCTGCAGGAGTCGGGCCCAGGACTGGTGAAGCCTTCGGAGA |
| | Chain | CCCTGTCCCTCACCTGCACTGTCTCTGGTGGCTCCATCAGTAGTTACTACTG |
| | | GACCTGGATCCGGCAGCCCGCCGGGAAGGGACTGGAGTGGATTGGGCGT |
| | | GTCTTTACCAGTGAGAGCACCAACTACAACCCCTCCCTCAAGAATCGAGTC |
| | | ACCATGTCAGTAGACACGTCCAAGAACCAGTTCTCCCTGAGGCTGAATTCT |
| | | GTGACCGCCGCGGACACGGCCATGTATTACTGTGCGAGAGACCGGGGGA |
| | | CCTACCTAGGGGGGTTCGACCCCTGGGGCCAGGGAACCCTGGTCACCGTC |
| | | TCCTCAGCCTCCACCAAGGGCCCATCGGTGTTTCCCCTGGCCCCCAGCAGC |
| | | AAGTCTACTTCCGGCGGAACTGCTGCCCTGGGTTGCCTGGTGAAGGACTA |
| | | CTTCCCCTGTCCCGTGACAGTGTCCTGGAACTCTGGGGCTCTGACTTCCGG |
| | | CGTGCACACCTTCCCCGCCGTGCTGCAGAGCAGCGGCCTGTACAGCCTGA |
| | | GCAGCGTGGTGACAGTGCCCTCCAGCTCTCTGGGAACCCAGACCTATATCT |
| | | GCAACGTGAACCACAAGCCCAGCAACACCAAGGTGGACAAGAGAGTGGA |
| | | GCCCAAGAGCTGCGACAAGACCCACACCTGCCCCCCCTGCCCAGCTCCAG |
| | | AACTGCTGGGAGGGCCTTCCGTGTTCCTGTTCCCCCCCAAGCCCAAGGACA |
| | | CCCTGATGATCAGCAGGACCCCCGAGGTGACCTGCGTGGTGGTGGCCGTG |
| | | TCCCACGAGGACCCAGAGGTGAAGTTCAACTGGTACGTGGACGGCGTGG |
| | | AGGTGCACAACGCCAAGACCAAGCCCAGAGAGGAGCAGTACAACAGCAC |
| | | CTACAGGGTGGTGTCCGTGCTGACCGTGCTGCACCAGGACTGGCTGAACG |
| | | GCAAAGAATACAAGTGCAAAGTCTCCAACAAGGCCCTGGCTGCCCCAATC |
| | | GAAAAGACAATCAGCAAGGCCAAGGGCCAGCCACGGGAGCCCCAGGTGT |
| | | ACACCCTGCCCCCCAGCCGGGAGGAGATGACCAAGAACCAGGTGTCCCTG |
| | | ACCTGTCTGGTGAAGGGCTTCTACCCCTGTGATATCGCCGTGGAGTGGGA |
| | | GAGCAACGGCCAGCCCGAGAACAACTACAAGACCACCCCCCCAGTGCTGG |
| | | ACAGCGACGGCAGCTTCTTCCTGTACAGCAAGCTGACCGTGGACAAGTCC |
| | | AGGTGGCAGCAGGGCAACGTGTTCAGCTGCAGCGTGATGCACGAGGCCC |
| | | TGCACAACCACTACACCCAGAAGTCCCTGAGCCTGAGCCCCGGCAAG |
| SEQ ID NO: 216 | LCDR1 (Combined) | RSSQSLLHSNGYNYLD |
| SEQ ID NO: 217 | LCDR2 (Combined) | LGSNRAS |
| SEQ ID NO: 238 | LCDR3 (Combined) | IQALQTPFT |
| SEQ ID NO: 216 | LCDR1 (Kabat) | RSSQSLLHSNGYNYLD |
| SEQ ID NO: 217 | LCDR2 (Kabat) | LGSNRAS |
| SEQ ID NO: 238 | LCDR3 (Kabat) | IQALQTPFT |
| SEQ ID NO: 219 | LCDR1 (Chothia) | SQSLLHSNGYNY |
| SEQ ID NO: 220 | LCDR2 (Chothia) | LGS |
| SEQ ID NO: 239 | LCDR3 (Chothia) | ALQTPF |
| SEQ ID NO: 222 | LCDR1 (IMGT) | QSLLHSNGYNY |
| SEQ ID NO: 220 | LCDR2 (IMGT) | LGS |
| SEQ ID NO: 238 | LCDR3 (IMGT) | IQALQTPFT |
| SEQ ID NO: 240 | VL | DIVMTQSPLSLPVTPGEPASISCRSSQSLLHSNGYNYLDWYLQKPGQSPQLLIY |
| | | LGSNRASGVPDRFSGSGSGTDFTLKISRVEAEDVGVYYCIQALQTPFTFGQGT |
| | | KLEIK |
| SEQ ID NO: 241 | DNA VL | GATATTGTGATGACTCAGTCTCCACTCTCCCTGCCCGTCACCCCTGGAGAG |
| | | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGCTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATACAAGCTCTACAAACTCCGTTCA |
| | | CTTTTGGCCAGGGGACCAAACTGGAGATCAAA |
| SEQ ID NO: 242 | Light Chain | DIVMTQSPLSLPVTPGEPASISCRSSQSLLHSNGYNYLDWYLQKPGQSPQLLIY |
| | | LGSNRASGVPDRFSGSGSGTDFTLKISRVEAEDVGVYYCIQALQTPFTFGQGT |
| | | KLEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKSF |
| | | NRGEC |
| SEQ ID NO: 243 | DNA Light | GATATTGTGATGACTCAGTCTCCACTCTCCCTGCCCGTCACCCCTGGAGAG |
| | Chain | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGCTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATACAAGCTCTACAAACTCCGTTCA |
| | | CTTTTGGCCAGGGGACCAAACTGGAGATCAAACGTACGGTGGCCGCTCCC |
| | | AGCGTGTTCATCTTCCCCCCCAGCGACGAGCAGCTGAAGAGTGGCACCGC |
| | | CAGCGTGGTGTGCCTGCTGAACAACTTCTACCCCCGGGAGGCCAAGGTGC |
| | | AGTGGAAGGTGGACAACGCCCTGCAGAGCGGCAACAGCCAGGAGAGCGT |
| | | CACCGAGCAGGACAGCAAGGACTCCACCTACAGCCTGAGCAGCACCCTGA |
| | | CCCTGAGCAAGGCCGACTACGAGAAGCATAAGGTGTACGCCTGCGAGGT |
| | | GACCCACCAGGGCCTGTCCAGCCCCGTGACCAAGAGCTTCAACAGGGGCG |
| | | AGTGC |
| 550E03 | | |
| Parental | | |
| SEQ ID NO: 244 | HCDR1 (Combined) | GFSLSTSGMSVS |
| SEQ ID NO: 26 | HCDR2 (Combined) | LIDWDDDKYYSTSLKT |
| SEQ ID NO: 245 | HCDR3 (Combined) | MALRHAFDA |
| SEQ ID NO: 141 | HCDR1 (Kabat) | TSGMSVS |
| SEQ ID NO: 26 | HCDR2 (Kabat) | LIDWDDDKYYSTSLKT |
| SEQ ID NO: 245 | HCDR3 (Kabat) | MALRHAFDA |
| SEQ ID NO: 246 | HCDR1 (Chothia) | GFSLSTSGM |
| SEQ ID NO: 30 | HCDR2 (Chothia) | DWDDD |
| SEQ ID NO: 245 | HCDR3 (Chothia) | MALRHAFDA |
| SEQ ID NO: 247 | HCDR1 (IMGT) | GFSLSTSGMS |
| SEQ ID NO: 32 | HCDR2 (IMGT) | IDWDDDK |
| SEQ ID NO: 248 | HCDR3 (IMGT) | ARMALRHAFDA |
| SEQ ID NO: 249 | VH | QVTLRESGPALVKPTQTLTLTCTFSGFSLSTSGMSVSWIRQPPGKALEWLALID |
| | | WDDDKYYSTSLKTRLTISKDTSKNQVVLTMTNMDPVDTATYYCARMALRHA |
| | | FDAWGQGTMVTVSS |
| SEQ ID NO: 250 | DNA VH | CAGGTCACCTTGAGGGAGTCTGGTCCTGCGCTGGTGAAGCCCACACAGAC |
| | | CCTCACACTGACCTGCACCTTCTCTGGGTTCTCACTCAGCACTAGTGGAAT |
| | | GTCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGATGGCA |
| | | CTACGTCATGCTTTTGATGCCTGGGGCCAAGGGACAATGGTCACCGTCTCT |
| | | TCA |
| SEQ ID NO: 251 | Heavy | QVTLRESGPALVKPTQTLTLTCTFSGFSLSTSGMSVSWIRQPPGKALEWLALID |
| | Chain | WDDDKYYSTSLKTRLTISKDTSKNQVVLTMTNMDPVDTATYYCARMALRHA |
| | | FDAWGQGTMVIVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 252 | DNA Heavy | CAGGTCACCTTGAGGGAGTCTGGTCCTGCGCTGGTGAAGCCCACACAGAC |
| | Chain | CCTCACACTGACCTGCACCTTCTCTGGGTTCTCACTCAGCACTAGTGGAAT |
| | | GTCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGATGGCA |
| | | CTACGTCATGCTTTTGATGCCTGGGGCCAAGGGACAATGGTCACCGTCTCT |
| | | TCAGCCTCCACCAAGGGCCCATCGGTGTTTCCCCTGGCCCCCAGCAGCAAG |
| | | TCTACTTCCGGCGGAACTGCTGCCCTGGGTTGCCTGGTGAAGGACTACTTC |
| | | CCCTGTCCCGTGACAGTGTCCTGGAACTCTGGGGCTCTGACTTCCGGCGTG |
| | | CACACCTTCCCCGCCGTGCTGCAGAGCAGCGGCCTGTACAGCCTGAGCAG |
| | | CGTGGTGACAGTGCCCTCCAGCTCTCTGGGAACCCAGACCTATATCTGCAA |
| | | CGTGAACCACAAGCCCAGCAACACCAAGGTGGACAAGAGAGTGGAGCCC |
| | | AAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: 253 | LCDR1 (Combined) | TGSSSNIGAGYDVH |
| SEQ ID NO: 254 | LCDR2 (Combined) | VNSNRPS |
| SEQ ID NO: 255 | LCDR3 (Combined) | QSYDSSLSGWV |
| SEQ ID NO: 253 | LCDR1 (Kabat) | TGSSSNIGAGYDVH |
| SEQ ID NO: 254 | LCDR2 (Kabat) | VNSNRPS |
| SEQ ID NO: 255 | LCDR3 (Kabat) | QSYDSSLSGWV |
| SEQ ID NO: 256 | LCDR1 (Chothia) | SSSNIGAGYD |
| SEQ ID NO: 257 | LCDR2 (Chothia) | VNS |
| SEQ ID NO: 258 | LCDR3 (Chothia) | YDSSLSGW |
| SEQ ID NO: 259 | LCDR1 (IMGT) | SSNIGAGYD |
| SEQ ID NO: 257 | LCDR2 (IMGT) | VNS |
| SEQ ID NO: 255 | LCDR3 (IMGT) | QSYDSSLSGWV |
| SEQ ID NO: 260 | VL | QSVLTQPPSVSGAPGQRVTISCTGSSSNIGAGYDVHWYQQLPGTAPKLLIYVN |
| | | SNRPSGVPDRFSGSKSGTSASLAITGLQAEDEADYYCQSYDSSLSGWVFGGGT |
| | | KLTVL |
| SEQ ID NO: 261 | DNA VL | CAGTCTGTGCTGACGCAGCCGCCCTCAGTGTCTGGGGCCCCAGGGCAGAG |
| | | GGTCACCATCTCCTGCACTGGGAGCAGCTCCAACATCGGGGCAGGTTATG |
| | | ATGTACACTGGTACCAGCAGCTTCCAGGAACAGCCCCCAAACTCCTCATCT |
| | | ATGTTAACAGCAATCGGCCCTCAGGGGTCCCTGACCGATTCTCTGGCTCCA |
| | | AGTCTGGCACCTCAGCCTCCCTGGCCATCACTGGGCTCCAGGCTGAGGAT |
| | | GAGGCTGATTATTACTGCCAGTCCTATGACAGCAGCCTGAGTGGTTGGGT |
| | | GTTCGGCGGAGGGACCAAGTTGACCGTCCTA |
| SEQ ID NO: 262 | Light Chain | QSVLTQPPSVSGAPGQRVTISCTGSSSNIGAGYDVHWYQQLPGTAPKLLIYVN |
| | | SNRPSGVPDRFSGSKSGTSASLAITGLQAEDEADYYCQSYDSSLSGWVFGGGT |
| | | KLTVLGQPKAAPSVTLFPPSSEELQANKATLVCLISDFYPGAVTVAWKADSSPV |
| | | KAGVETTTPSKQSNNKYAASSYLSLTPEQWKSHRSYSCQVTHEGSTVEKTVAP |
| | | TECS |
| SEQ ID NO: 263 | DNA Light | CAGTCTGTGCTGACGCAGCCGCCCTCAGTGTCTGGGGCCCCAGGGCAGAG |
| | Chain | GGTCACCATCTCCTGCACTGGGAGCAGCTCCAACATCGGGGCAGGTTATG |
| | | ATGTACACTGGTACCAGCAGCTTCCAGGAACAGCCCCCAAACTCCTCATCT |
| | | ATGTTAACAGCAATCGGCCCTCAGGGGTCCCTGACCGATTCTCTGGCTCCA |
| | | AGTCTGGCACCTCAGCCTCCCTGGCCATCACTGGGCTCCAGGCTGAGGAT |
| | | GAGGCTGATTATTACTGCCAGTCCTATGACAGCAGCCTGAGTGGTTGGGT |
| | | GTTCGGCGGAGGGACCAAGTTGACCGTCCTAGGTCAGCCCAAGGCTGCCC |
| | | CCTCCGTGACCCTGTTCCCCCCCAGCTCCGAGGAACTGCAGGCCAACAAG |
| | | GCCACCCTGGTGTGCCTGATCAGCGACTTCTACCCTGGCGCCGTGACCGTG |
| | | GCCTGGAAGGCCGACAGCAGCCCCGTGAAGGCCGGCGTGGAGACAACCA |
| | | CCCCCAGCAAGCAGAGCAACAACAAGTACGCCGCCAGCAGCTACCTGAGC |
| | | CTGACCCCCGAGCAGTGGAAGAGCCACAGAAGCTACAGCTGCCAGGTCAC |
| | | CCACGAGGGCAGCACCGTGGAGAAAACCGTGGCCCCCACCGAGTGCAGC |
| 1G12 Hz | | |
| SEQ ID NO: 264 | HCDR1 (Combined) | GFSLTNYGVH |
| SEQ ID NO: 265 | HCDR2 (Combined) | VIWRGESTDYNAAFMS |
| SEQ ID NO: 266 | HCDR3 (Combined) | NGGSSGWYFDV |
| SEQ ID NO: 267 | HCDR1 (Kabat) | NYGVH |
| SEQ ID NO: 265 | HCDR2 (Kabat) | VIWRGESTDYNAAFMS |
| SEQ ID NO: 266 | HCDR3 (Kabat) | NGGSSGWYFDV |
| SEQ ID NO: 268 | HCDR1 (Chothia) | GFSLTNY |
| SEQ ID NO: 269 | HCDR2 (Chothia) | WRGES |
| SEQ ID NO: 266 | HCDR3 (Chothia) | NGGSSGWYFDV |
| SEQ ID NO: 270 | HCDR1 (IMGT) | GFSLTNYG |
| SEQ ID NO: 271 | HCDR2 (IMGT) | IWRGEST |
| SEQ ID NO: 272 | HCDR3 (IMGT) | ARNGGSSGWYFDV |
| SEQ ID NO: 273 | VH | QVQLQESGPGLVKPSETLSLTCTVSGFSLTNYGVHWIRQPPGKGLEWIGVIW |
| | | RGESTDYNAAFMSRVTISKDDSKSQVSLKLSSVTAADTAVYYCARNGGSSGW |
| | | YFDVWGQGTTVTVSS |
| SEQ ID NO: 274 | DNA VH | CAAGTTCAGCTGCAAGAATCTGGCCCTGGCCTGGTCAAGCCTTCCGAGAC |
| | | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGGCGGA |
| | | TCCTCCGGCTGGTACTTTGATGTGTGGGGCCAGGGCACCACCGTGACAGT |
| | | TAGTTCT |
| SEQ ID NO: 275 | Heavy | QVQLQESGPGLVKPSETLSLTCTVSGFSLTNYGVHWIRQPPGKGLEWIGVIW |
| | Chain | RGESTDYNAAFMSRVTISKDDSKSQVSLKLSSVTAADTAVYYCARNGGSSGW |
| | | YFDVWGQGTTVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 276 | DNA Heavy | CAAGTTCAGCTGCAAGAATCTGGCCCTGGCCTGGTCAAGCCTTCCGAGAC |
| | Chain | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGGCGGA |
| | | TCCTCCGGCTGGTACTTTGATGTGTGGGGCCAGGGCACCACCGTGACAGT |
| | | TAGTTCTGCTAGCACCAAGGGCCCAAGTGTGTTTCCCCTGGCCCCCAGCAG |
| | | CAAGTCTACTTCCGGCGGAACTGCTGCCCTGGGTTGCCTGGTGAAGGACT |
| | | ACTTCCCCTGTCCCGTGACAGTGTCCTGGAACTCTGGGGCTCTGACTTCCG |
| | | GCGTGCACACCTTCCCCGCCGTGCTGCAGAGCAGCGGCCTGTACAGCCTG |
| | | AGCAGCGTGGTGACAGTGCCCTCCAGCTCTCTGGGAACCCAGACCTATAT |
| | | CTGCAACGTGAACCACAAGCCCAGCAACACCAAGGTGGACAAGAGAGTG |
| | | GAGCCCAAGAGCTGCGACAAGACCCACACCTGCCCCCCCTGCCCAGCTCC |
| | | AGAACTGCTGGGAGGGCCTTCCGTGTTCCTGTTCCCCCCCAAGCCCAAGG |
| | | ACACCCTGATGATCAGCAGGACCCCCGAGGTGACCTGCGTGGTGGTGGCC |
| | | GTGTCCCACGAGGACCCAGAGGTGAAGTTCAACTGGTACGTGGACGGCG |
| | | TGGAGGTGCACAACGCCAAGACCAAGCCCAGAGAGGAGCAGTACAACAG |
| | | CACCTACAGGGTGGTGTCCGTGCTGACCGTGCTGCACCAGGACTGGCTGA |
| | | ACGGCAAAGAATACAAGTGCAAAGTCTCCAACAAGGCCCTGGCTGCCCCA |
| | | ATCGAAAAGACAATCAGCAAGGCCAAGGGCCAGCCACGGGAGCCCCAGG |
| | | TGTACACCCTGCCCCCCAGCCGGGAGGAGATGACCAAGAACCAGGTGTCC |
| | | CTGACCTGTCTGGTGAAGGGCTTCTACCCCTGTGATATCGCCGTGGAGTG |
| | | GGAGAGCAACGGCCAGCCCGAGAACAACTACAAGACCACCCCCCCAGTGC |
| | | TGGACAGCGACGGCAGCTTCTTCCTGTACAGCAAGCTGACCGTGGACAAG |
| | | TCCAGGTGGCAGCAGGGCAACGTGTTCAGCTGCAGCGTGATGCACGAGG |
| | | CCCTGCACAACCACTACACCCAGAAGTCCCTGAGCCTGAGCCCCGGCAAG |
| SEQ ID NO: 277 | LCDR1 (Combined) | KASQDVTSAVA |
| SEQ ID NO: 278 | LCDR2 (Combined) | WTSTRHT |
| SEQ ID NO: 279 | LCDR3 (Combined) | QQHYTTPLT |
| SEQ ID NO: 277 | LCDR1 (Kabat) | KASQDVTSAVA |
| SEQ ID NO: 278 | LCDR2 (Kabat) | WTSTRHT |
| SEQ ID NO: 279 | LCDR3 (Kabat) | QQHYTTPLT |
| SEQ ID NO: 280 | LCDR1 (Chothia) | SQDVTSA |
| SEQ ID NO: 281 | LCDR2 (Chothia) | WTS |
| SEQ ID NO: 282 | LCDR3 (Chothia) | HYTTPL |
| SEQ ID NO: 283 | LCDR1 (IMGT) | QDVTSA |
| SEQ ID NO: 281 | LCDR2 (IMGT) | WTS |
| SEQ ID NO: 279 | LCDR3 (IMGT) | QQHYTTPLT |
| SEQ ID NO: 284 | VL | DIQLTQSPSFLSASVGDRVTITCKASQDVTSAVAWYQQKPGKAPKLLIYWTST |
| | | RHTGVPSRFSGSGSGTEYTLTISSLQPEDFATYYCQQHYTTPLTFGQGTKLEIK |
| SEQ ID NO: 285 | DNA VL | GATATTCAGCTGACCCAGTCTCCTAGCTTCCTGTCCGCTTCTGTGGGCGAC |
| | | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAG |
| SEQ ID NO: 286 | Light Chain | DIQLTQSPSFLSASVGDRVTITCKASQDVTSAVAWYQQKPGKAPKLLIYWTST |
| | | RHTGVPSRFSGSGSGTEYTLTISSLQPEDFATYYCQQHYTTPLTFGQGTKLEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 287 | DNA Light | GATATTCAGCTGACCCAGTCTCCTAGCTTCCTGTCCGCTTCTGTGGGCGAC |
| | Chain | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAGCGTACGGTGGCCGCTCCCAGCGTGTTCATCTTCC |
| | | CCCCCAGCGACGAGCAGCTGAAGAGTGGCACCGCCAGCGTGGTGTGCCT |
| | | GCTGAACAACTTCTACCCCCGGGAGGCCAAGGTGCAGTGGAAGGTGGAC |
| | | AACGCCCTGCAGAGCGGCAACAGCCAGGAGAGCGTCACCGAGCAGGACA |
| | | GCAAGGACTCCACCTACAGCCTGAGCAGCACCCTGACCCTGAGCAAGGCC |
| | | GACTACGAGAAGCATAAGGTGTACGCCTGCGAGGTGACCCACCAGGGCC |
| | | TGTCCAGCCCCGTGACCAAGAGCTTCAACAGGGGCGAGTGC |
| 1G12 mouse | | |
| SEQ ID NO: 264 | HCDR1 (Combined) | GFSLTNYGVH |
| SEQ ID NO: 265 | HCDR2 (Combined) | VIWRGESTDYNAAFMS |
| SEQ ID NO: 266 | HCDR3 (Combined) | NGGSSGWYFDV |
| SEQ ID NO: 267 | HCDR1 (Kabat) | NYGVH |
| SEQ ID NO: 265 | HCDR2 (Kabat) | VIWRGESTDYNAAFMS |
| SEQ ID NO: 266 | HCDR3 (Kabat) | NGGSSGWYFDV |
| SEQ ID NO: 268 | HCDR1 (Chothia) | GFSLTNY |
| SEQ ID NO: 269 | HCDR2 (Chothia) | WRGES |
| SEQ ID NO: 266 | HCDR3 (Chothia) | NGGSSGWYFDV |
| SEQ ID NO: 270 | HCDR1 (IMGT) | GFSLTNYG |
| SEQ ID NO: 271 | HCDR2 (IMGT) | IWRGEST |
| SEQ ID NO: 272 | HCDR3 (IMGT) | ARNGGSSGWYFDV |
| SEQ ID NO: 288 | VH | QVQLQQSGPGLVQPSQSLSITCTVSGFSLTNYGVHWVRQSPGKGLEWLGVI |
| | | WRGESTDYNAAFMSRLSVTKDDSKSQVFFKMNSLQADDTAIYYCARNGGSS |
| | | GWYFDVWGTGTTVTVSS |
| SEQ ID NO: 289 | DNA VH | CAGGTGCAGCTACAGCAGTCAGGACCTGGCCTAGTGCAGCCCTCACAGAG |
| | | CCTGTCCATAACCTGCACAGTCTCTGGTTTCTCATTAACTAACTATGGTGTA |
| | | CACTGGGTTCGCCAGTCTCCAGGAAAGGGTCTGGAGTGGCTGGGAGTGA |
| | | TATGGAGAGGTGAAAGCACAGACTACAATGCAGCTTTCATGTCCAGACTG |
| | | AGCGTCACCAAGGACGACTCCAAGAGCCAAGTTTTCTTTAAAATGAACAG |
| | | TCTGCAAGCTGATGACACTGCCATATACTACTGTGCCAGAAATGGCGGTA |
| | | GTAGCGGGTGGTACTTCGATGTCTGGGGCACAGGGACCACTGTCACCGTC |
| | | TCCTCA |
| SEQ ID NO: 290 | Heavy | QVQLQQSGPGLVQPSQSLSITCTVSGFSLTNYGVHWVRQSPGKGLEWLGVI |
| | Chain | WRGESTDYNAAFMSRLSVTKDDSKSQVFFKMNSLQADDTAIYYCARNGGSS |
| | | GWYFDVWGTGTTVTVSSAKTTAPSVYPLAPVCGGTTGSSVTLGCLVKGYFPC |
| | | PVTLTWNSGSLSSGVHTFPALLQSGLYTLSSSVTVTSNTWPSQTITCNVAHPA |
| | | SSTKVDKKIEPRVPITQNPCPPLKECPPCAAPDLLGGPSVFIFPPKIKDVLMISLS |
| | | PMVTCVVVAVSEDDPDVQISWFVNNVEVHTAQTQTHREDYNSTLRVVSALP |
| | | IQHQDWMSGKEFKCKVNNRALASPIEKTISKPRGPVRAPQVYVLPPPAEEMT |
| | | KKEFSLTCMITGFLPCEIAVDWTSNGRTEQNYKNTATVLDSDGSYFMYSKLRV |
| | | QKSTWERGSLFACSVVHEGLHNHLTTKTISRSLGK |
| SEQ ID NO: 291 | DNA Heavy | CAGGTGCAGCTACAGCAGTCAGGACCTGGCCTAGTGCAGCCCTCACAGAG |
| | Chain | CCTGTCCATAACCTGCACAGTCTCTGGTTTCTCATTAACTAACTATGGTGTA |
| | | CACTGGGTTCGCCAGTCTCCAGGAAAGGGTCTGGAGTGGCTGGGAGTGA |
| | | TATGGAGAGGTGAAAGCACAGACTACAATGCAGCTTTCATGTCCAGACTG |
| | | AGCGTCACCAAGGACGACTCCAAGAGCCAAGTTTTCTTTAAAATGAACAG |
| | | TCTGCAAGCTGATGACACTGCCATATACTACTGTGCCAGAAATGGCGGTA |
| | | GTAGCGGGTGGTACTTCGATGTCTGGGGCACAGGGACCACTGTCACCGTC |
| | | TCCTCAGCCAAAACAACAGCCCCATCGGTCTATCCACTGGCCCCTGTGTGT |
| | | GGAGGTACAACTGGCTCCTCGGTGACTCTAGGATGCCTGGTCAAGGGTTA |
| | | TTTCCCTTGTCCAGTGACCTTGACCTGGAACTCTGGATCCCTGTCCAGTGGT |
| | | GTGCACACCTTCCCAGCTCTCCTGCAGTCTGGCCTCTACACCCTCAGCAGCT |
| | | CAGTGACTGTAACCTCGAACACCTGGCCCAGCCAGACCATCACCTGCAATG |
| | | TGGCCCACCCGGCAAGCAGCACCAAAGTGGACAAGAAAATTGAGCCCAG |
| | | AGTGCCCATAACACAGAACCCCTGTCCTCCACTCAAAGAGTGTCCCCCATG |
| | | CGCAGCTCCAGACCTCTTGGGTGGACCATCCGTCTTCATCTTCCCTCCAAA |
| | | GATCAAGGATGTACTCATGATCTCCCTGAGCCCCATGGTCACATGTGTGGT |
| | | GGTGGCTGTGAGCGAGGATGACCCAGACGTCCAGATCAGCTGGTTTGTGA |
| | | ACAACGTGGAAGTACACACAGCTCAGACACAAACCCATAGAGAGGATTAC |
| | | AACAGTACTCTCCGGGTGGTCAGTGCCCTCCCCATCCAGCACCAGGACTG |
| | | GATGAGTGGCAAGGAGTTCAAATGCAAGGTCAACAACAGAGCCCTCGCAT |
| | | CCCCCATCGAGAAAACCATCTCAAAACCCAGAGGGCCAGTAAGAGCTCCA |
| | | CAGGTATATGTCTTGCCTCCACCAGCAGAAGAGATGACTAAGAAAGAGTT |
| | | CAGTCTGACCTGCATGATCACAGGCTTCTTACCTTGTGAAATTGCTGTGGA |
| | | CTGGACCAGCAATGGGCGTACAGAGCAAAACTACAAGAACACCGCAACA |
| | | GTCCTGGACTCTGATGGTTCTTACTTCATGTACAGCAAGCTCAGAGTACAA |
| | | AAGAGCACTTGGGAAAGAGGAAGTCTTTTCGCCTGCTCAGTGGTCCACGA |
| | | GGGTCTGCACAATCACCTTACGACTAAGACCATCTCCCGGTCTCTGGGTAA |
| | | A |
| SEQ ID NO: 277 | LCDR1 (Combined) | KASQDVTSAVA |
| SEQ ID NO: 278 | LCDR2 (Combined) | WTSTRHT |
| SEQ ID NO: 279 | LCDR3 (Combined) | QQHYTTPLT |
| SEQ ID NO: 277 | LCDR1 (Kabat) | KASQDVTSAVA |
| SEQ ID NO: 278 | LCDR2 (Kabat) | WTSTRHT |
| SEQ ID NO: 279 | LCDR3 (Kabat) | QQHYTTPLT |
| SEQ ID NO: 280 | LCDR1 (Chothia) | SQDVTSA |
| SEQ ID NO: 281 | LCDR2 (Chothia) | WTS |
| SEQ ID NO: 282 | LCDR3 (Chothia) | HYTTPL |
| SEQ ID NO: 283 | LCDR1 (IMGT) | QDVTSA |
| SEQ ID NO: 281 | LCDR2 (IMGT) | WTS |
| SEQ ID NO: 279 | LCDR3 (IMGT) | QQHYTTPLT |
| SEQ ID NO: 292 | VL | DIQMTQTHKFMSTSVGDRVSITCKASQDVTSAVAWYQQTPGQSPNLLIYWT |
| | | STRHTGVPDRFTGSGSGTDYTLTISSVQAEDLALYYCQQHYTTPLTFGAGTKLE |
| | | LK |
| SEQ ID NO: 293 | DNA VL | GACATTCAGATGACCCAGACTCACAAATTCATGTCCACATCAGTAGGAGAC |
| | | AGGGTCAGCATCACCTGCAAGGCCAGTCAGGATGTGACTTCTGCTGTAGC |
| | | CTGGTATCAACAAACACCAGGACAATCTCCTAATCTACTGATTTACTGGAC |
| | | ATCCACCCGGCACACTGGAGTCCCTGATCGCTTCACAGGCAGTGGATCTG |
| | | GGACAGATTATACTCTCACCATCAGCAGTGTGCAGGCTGAAGACCTGGCA |
| | | CTTTATTACTGTCAGCAACATTATACCACTCCGCTCACGTTCGGTGCTGGGA |
| | | CCAAGCTGGAGCTAAAA |
| SEQ ID NO: 294 | Light Chain | DIQMTQTHKFMSTSVGDRVSITCKASQDVTSAVAWYQQTPGQSPNLLIYWT |
| | | STRHTGVPDRFTGSGSGTDYTLTISSVQAEDLALYYCQQHYTTPLTFGAGTKLE |
| | | LKRADAAPTVSIFPPSSEQLTSGGASVVCFLNNFYPKDINVKWKIDGSERQNG |
| | | VLNSWTDQDSKDSTYSMSSTLTLTKDEYERHNSYTCEATHKTSTSPIVKSFNR |
| | | NEC |
| SEQ ID NO: 295 | DNA Light | GACATTCAGATGACCCAGACTCACAAATTCATGTCCACATCAGTAGGAGAC |
| | Chain | AGGGTCAGCATCACCTGCAAGGCCAGTCAGGATGTGACTTCTGCTGTAGC |
| | | CTGGTATCAACAAACACCAGGACAATCTCCTAATCTACTGATTTACTGGAC |
| | | ATCCACCCGGCACACTGGAGTCCCTGATCGCTTCACAGGCAGTGGATCTG |
| | | GGACAGATTATACTCTCACCATCAGCAGTGTGCAGGCTGAAGACCTGGCA |
| | | CTTTATTACTGTCAGCAACATTATACCACTCCGCTCACGTTCGGTGCTGGGA |
| | | CCAAGCTGGAGCTAAAACGTGCCGATGCTGCACCAACTGTATCCATCTTCC |
| | | CACCATCCAGTGAGCAGTTAACATCTGGAGGTGCCTCAGTCGTGTGCTTCT |
| | | TGAACAACTTCTACCCCAAAGACATCAATGTCAAGTGGAAGATTGATGGC |
| | | AGTGAACGACAAAATGGCGTCCTGAACAGTTGGACTGATCAGGACAGCA |
| | | AAGACAGCACCTACAGCATGAGCAGCACCCTCACGTTGACCAAGGACGAG |
| | | TATGAACGACATAACAGCTATACCTGTGAGGCCACTCACAAGACATCAACT |
| | | TCACCCATTGTCAAGAGCTTCAACAGGAATGAGTGT |
| 1G12 | | |
| SEQ ID NO: 264 | HCDR1 (Combined) | GFSLTNYGVH |
| SEQ ID NO: 265 | HCDR2 (Combined) | VIWRGESTDYNAAFMS |
| SEQ ID NO: 296 | HCDR3 (Combined) | NAGSSGWYFDV |
| SEQ ID NO: 267 | HCDR1 (Kabat) | NYGVH |
| SEQ ID NO: 265 | HCDR2 (Kabat) | VIWRGESTDYNAAFMS |
| SEQ ID NO: 296 | HCDR3 (Kabat) | NAGSSGWYFDV |
| SEQ ID NO: 268 | HCDR1 (Chothia) | GFSLTNY |
| SEQ ID NO: 269 | HCDR2 (Chothia) | WRGES |
| SEQ ID NO: 296 | HCDR3 (Chothia) | NAGSSGWYFDV |
| SEQ ID NO: 270 | HCDR1 (IMGT) | GFSLTNYG |
| SEQ ID NO: 271 | HCDR2 (IMGT) | IWRGEST |
| SEQ ID NO: 297 | HCDR3 (IMGT) | ARNAGSSGWYFDV |
| SEQ ID NO: 298 | VH | QVQLQESGPGLVKPSETLSLTCTVSGFSLTNYGVHWIRQPPGKGLEWIGVIW |
| | | RGESTDYNAAFMSRVTISKDDSKSQVSLKLSSVTAADTAVYYCARNAGSSGW |
| | | YFDVWGQGTTVTVSS |
| SEQ ID NO: 299 | DNA VH | CAAGTTCAGCTGCAAGAATCTGGCCCTGGCCTGGTCAAGCCTTCCGAGAC |
| | | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGCTGGCT |
| | | CCTCCGGCTGGTACTTTGATGTGTGGGGCCAGGGCACCACCGTGACAGTT |
| | | AGTTCT |
| SEQ ID NO: 300 | Heavy | QVQLQESGPGLVKPSETLSLTCTVSGFSLTNYGVHWIRQPPGKGLEWIGVIW |
| | Chain | RGESTDYNAAFMSRVTISKDDSKSQVSLKLSSVTAADTAVYYCARNAGSSGW |
| | | YFDVWGQGTTVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: 301 | DNA Heavy | CAAGTTCAGCTGCAAGAATCTGGCCCTGGCCTGGTCAAGCCTTCCGAGAC |
| | Chain | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGCTGGCT |
| | | CCTCCGGCTGGTACTTTGATGTGTGGGGCCAGGGCACCACCGTGACAGTT |
| | | AGTTCTGCTAGCACCAAGGGCCCAAGTGTGTTTCCCCTGGCCCCCAGCAGC |
| | | AAGTCTACTTCCGGCGGAACTGCTGCCCTGGGTTGCCTGGTGAAGGACTA |
| | | CTTCCCCTGTCCCGTGACAGTGTCCTGGAACTCTGGGGCTCTGACTTCCGG |
| | | CGTGCACACCTTCCCCGCCGTGCTGCAGAGCAGCGGCCTGTACAGCCTGA |
| | | GCAGCGTGGTGACAGTGCCCTCCAGCTCTCTGGGAACCCAGACCTATATCT |
| | | GCAACGTGAACCACAAGCCCAGCAACACCAAGGTGGACAAGAGAGTGGA |
| | | GCCCAAGAGCTGCGACAAGACCCACACCTGCCCCCCCTGCCCAGCTCCAG |
| | | AACTGCTGGGAGGGCCTTCCGTGTTCCTGTTCCCCCCCAAGCCCAAGGACA |
| | | CCCTGATGATCAGCAGGACCCCCGAGGTGACCTGCGTGGTGGTGGCCGTG |
| | | TCCCACGAGGACCCAGAGGTGAAGTTCAACTGGTACGTGGACGGCGTGG |
| | | AGGTGCACAACGCCAAGACCAAGCCCAGAGAGGAGCAGTACAACAGCAC |
| | | CTACAGGGTGGTGTCCGTGCTGACCGTGCTGCACCAGGACTGGCTGAACG |
| | | GCAAAGAATACAAGTGCAAAGTCTCCAACAAGGCCCTGGCTGCCCCAATC |
| | | GAAAAGACAATCAGCAAGGCCAAGGGCCAGCCACGGGAGCCCCAGGTGT |
| | | ACACCCTGCCCCCCAGCCGGGAGGAGATGACCAAGAACCAGGTGTCCCTG |
| | | ACCTGTCTGGTGAAGGGCTTCTACCCCTGTGATATCGCCGTGGAGTGGGA |
| | | GAGCAACGGCCAGCCCGAGAACAACTACAAGACCACCCCCCCAGTGCTGG |
| | | ACAGCGACGGCAGCTTCTTCCTGTACAGCAAGCTGACCGTGGACAAGTCC |
| | | AGGTGGCAGCAGGGCAACGTGTTCAGCTGCAGCGTGATGCACGAGGCCC |
| | | TGCACAACCACTACACCCAGAAGTCCCTGAGCCTGAGCCCCGGCAAG |
| SEQ ID NO: 277 | LCDR1 (Combined) | KASQDVTSAVA |
| SEQ ID NO: 278 | LCDR2 (Combined) | WTSTRHT |
| SEQ ID NO: 279 | LCDR3 (Combined) | QQHYTTPLT |
| SEQ ID NO: 277 | LCDR1 (Kabat) | KASQDVTSAVA |
| SEQ ID NO: 278 | LCDR2 (Kabat) | WTSTRHT |
| SEQ ID NO: 279 | LCDR3 (Kabat) | QQHYTTPLT |
| SEQ ID NO: 280 | LCDR1 (Chothia) | SQDVTSA |
| SEQ ID NO: 281 | LCDR2 (Chothia) | WTS |
| SEQ ID NO: 282 | LCDR3 (Chothia) | HYTTPL |
| SEQ ID NO: 283 | LCDR1 (IMGT) | QDVTSA |
| SEQ ID NO: 281 | LCDR2 (IMGT) | WTS |
| SEQ ID NO: 279 | LCDR3 (IMGT) | QQHYTTPLT |
| SEQ ID NO: 284 | VL | DIQLTQSPSFLSASVGDRVTITCKASQDVTSAVAWYQQKPGKAPKLLIYWTST |
| | | RHTGVPSRFSGSGSGTEYTLTISSLQPEDFATYYCQQHYTTPLTFGQGTKLEIK |
| SEQ ID NO: 285 | DNA VL | GATATTCAGCTGACCCAGTCTCCTAGCTTCCTGTCCGCTTCTGTGGGCGAC |
| | | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAG |
| SEQ ID NO: 286 | Light Chain | DIQLTQSPSFLSASVGDRVTITCKASQDVTSAVAWYQQKPGKAPKLLIYWTST |
| | | RHTGVPSRFSGSGSGTEYTLTISSLQPEDFATYYCQQHYTTPLTFGQGTKLEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: 287 | DNA Light | GATATTCAGCTGACCCAGTCTCCTAGCTTCCTGTCCGCTTCTGTGGGCGAC |
| | Chain | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAGCGTACGGTGGCCGCTCCCAGCGTGTTCATCTTCC |
| | | CCCCCAGCGACGAGCAGCTGAAGAGTGGCACCGCCAGCGTGGTGTGCCT |
| | | GCTGAACAACTTCTACCCCCGGGAGGCCAAGGTGCAGTGGAAGGTGGAC |
| | | AACGCCCTGCAGAGCGGCAACAGCCAGGAGAGCGTCACCGAGCAGGACA |
| | | GCAAGGACTCCACCTACAGCCTGAGCAGCACCCTGACCCTGAGCAAGGCC |
| | | GACTACGAGAAGCATAAGGTGTACGCCTGCGAGGTGACCCACCAGGGCC |
| | | TGTCCAGCCCCGTGACCAAGAGCTTCAACAGGGGCGAGTGC |

Other anti-DC-SIGN antibodies or antibody fragments (e.g., antigen binding fragments) disclosed herein include amino acids that have been mutated, yet have at least 80, 85, 90, 95, 96, 97, 98, or 99 percent identity in the CDR regions with the CDR regions depicted in the sequences described in Table 1. In some embodiments, it includes mutant amino acid sequences wherein no more than 1, 2, 3, 4 or 5 amino acids have been mutated in the CDR regions when compared with the CDR regions depicted in the sequence described in Table 1.

Also provided herein are nucleic acid sequences that encode VH, VL, full length heavy chain, and full length light chain of antibodies and antigen binding fragments thereof that specifically bind to DC-SIGN, e.g., the nucleic acid sequences in Table 1. Such nucleic acid sequences can be optimized for expression in mammalian cells.

Other anti-DC-SIGN antibodies disclosed herein include those where the amino acids or nucleic acids encoding the amino acids have been mutated, yet have at least 80, 85, 90 95, 96, 97, 98, or 99 percent identity to the sequences described in Table 1. In some embodiments, antibodies or antigen binding fragments thereof include mutant amino acid sequences wherein no more than 1, 2, 3, 4 or 5 amino acids have been mutated in the variable regions when compared with the variable regions depicted in the sequence described in Table 1, while retaining substantially the same therapeutic activity.

Since each provided antibody binds to DC-SIGN, the VH, VL, full length light chain, and full length heavy chain sequences (amino acid sequences and the nucleotide sequences encoding the amino acid sequences) can be “mixed and matched” to create other DC-SIGN-binding antibodies disclosed herein. Such “mixed and matched” DC-SIGN-binding antibodies can be tested using binding assays known in the art (e.g., ELISAs, assays described in the Exemplification). When chains are mixed and matched, a VH sequence from a particular VH/VL pairing should be replaced with a structurally similar VH sequence. A full length heavy chain sequence from a particular full length heavy chain/full length light chain pairing should be replaced with a structurally similar full length heavy chain sequence. A VL sequence from a particular VH/VL pairing should be replaced with a structurally similar VL sequence. A full length light chain sequence from a particular full length heavy chain/full length light chain pairing should be replaced with a structurally similar full length light chain sequence.

Accordingly, in one embodiment, the invention provides an isolated monoclonal antibody or antigen binding region thereof having: a heavy chain variable region comprising an amino acid sequence of SEQ ID NO: 10; and a light chain variable region comprising an amino acid sequence of SEQ ID NO: 21; wherein the antibody specifically binds to DC-SIGN. In one embodiment, the invention provides an isolated monoclonal antibody or antigen binding region thereof having: a heavy chain variable region comprising an amino acid sequence of SEQ ID NO: 34; and a light chain variable region comprising an amino acid sequence of SEQ ID NO: 45; wherein the antibody specifically binds to DC-SIGN. In one embodiment, the invention provides an isolated monoclonal antibody or antigen binding region thereof having: a heavy chain variable region comprising an amino acid sequence of SEQ ID NO: 55; and a light chain variable region comprising an amino acid sequence of SEQ ID NO: 64; wherein the antibody specifically binds to DC-SIGN. In another embodiment, the invention provides (i) an isolated monoclonal antibody having: a full length heavy chain comprising an amino acid sequence of any of SEQ ID NOs: 12, 36 or 57; and a full length light chain comprising an amino acid sequence of any of SEQ ID NOs: 23, 47 or 66; or (ii) a functional protein comprising an antigen binding portion thereof.

In another embodiment, the present disclosure provides DC-SIGN-binding antibodies that comprise the heavy chain CDR1, CDR2 and CDR3 and light chain CDR1, CDR2 and CDR3 as described in Table 1, or combinations thereof. The amino acid sequences of the VH CDR1s of the antibodies are shown in SEQ ID NOs: 1, 4, 5, 7, 25, 28, 29 and 31. The amino acid sequences of the VH CDR2 s of the antibodies and are shown in SEQ ID NOs: 2, 6, 8, 26, 30 and 32. The amino acid sequences of the VH CDR3 s of the antibodies are shown in SEQ ID NO: 3, 9, 27 and 33. The amino acid sequences of the VL CDR1 s of the antibodies are shown in SEQ ID NOs: 14, 17, 20, 38, 41 and 44. The amino acid sequences of the VL CDR2 s of the antibodies are shown in SEQ ID Nos: 15, 18, 39 and 42. The amino acid sequences of the VL CDR3 s of the antibodies are shown in SEQ ID NOs: 16, 19, 40 and 43.

Given that each of the antibodies binds DC-SIGN and that antigen-binding specificity is provided primarily by the CDR1, CDR2 and CDR3 regions, the VH CDR1, CDR2 and CDR3 sequences and VL CDR1, CDR2 and CDR3 sequences can be “mixed and matched” (i.e., CDRs from different antibodies can be mixed and match, although each antibody must contain a VH CDR1, CDR2 and CDR3 and a VL CDR1, CDR2 and CDR3 to create other DC-SIGN-binding molecules disclosed herein. Such “mixed and matched” DC-SIGN-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from CDR sequences shown herein for monoclonal antibodies of the present disclosure.

Accordingly, the present disclosure provides an isolated monoclonal antibody or antigen binding region thereof comprising a heavy chain CDR1 comprising an amino acid sequence selected from the group consisting of SEQ ID NOs: 1, 25, 49, 74, 88, 111, 138, 153, 178, 203, 227, 244, and 264; a heavy chain CDR2 comprising an amino acid sequence selected from the group consisting of SEQ ID NOs: 2, 26, 139, 154, 179, 204, 228, and 265; a heavy chain CDR3 comprising an amino acid sequence of SEQ ID NO: 3, 27, 50, 140, 155, 180, 205, 229, 245, and 266; a light chain CDR1 comprising an amino acid sequence selected from the group consisting of SEQ ID NOs: 14, 38, 59, 94, 166, 191, 216, 253, and 277; a light chain CDR2 comprising an amino acid sequence selected from the group consisting of SEQ ID NOs: 15, 39, 95, 167, 192, 217, 254, and 278; and a light chain CDR3 comprising an amino acid sequence selected from the group consisting of SEQ ID NOs: 16, 40, 60, 68, 82, 118, 124, 168, 193, 218, 238, 255, and 279; wherein the antibody specifically binds DC-SIGN.

In certain embodiments, an antibody that specifically binds to DC-SIGN is an antibody or antibody fragment (e.g., antigen binding fragment) that is described in Table 1.

In some embodiments, the antibody that specifically binds to human DC-SIGN comprises a heavy chain complementary determining region 1 (HCDR1) comprising the amino acid sequence of SEQ ID NO: 1; a heavy chain complementary determining region 2 (HCDR2) comprising the amino acid sequence of SEQ ID NO: 2; a heavy chain complementary determining region 3 (HCDR3) comprising the amino acid sequence of SEQ ID NO: 3; a light chain complementary determining region 1 (LCDR1) comprising the amino acid sequence of SEQ ID NO: 14; a light chain complementary determining region 2 (LCDR2) comprising the amino acid sequence of SEQ ID NO: 15; and a light chain complementary determining region 3 (LCDR3) comprising the amino acid sequence of SEQ ID NO: 16.

In some embodiments, the antibody that specifically binds to human DC-SIGN comprises a HCDR1 comprising the amino acid sequence of SEQ ID NO: 4; a HCDR2 comprising the amino acid sequence of SEQ ID NO: 2; a HCDR3 comprising the amino acid sequence of SEQ ID NO: 3; a LCDR1 comprising the amino acid sequence of SEQ ID NO: 14; a LCDR2 comprising the amino acid sequence of SEQ ID NO: 15; and a LCDR3 comprising the amino acid sequence of SEQ ID NO: 16.

In some embodiments, the antibody that specifically binds to human DC-SIGN comprises a HCDR1 comprising the amino acid sequence of SEQ ID NO: 5; a HCDR2 comprising the amino acid sequence of SEQ ID NO: 6; a HCDR3 comprising the amino acid sequence of SEQ ID NO: 3; a LCDR1 comprising the amino acid sequence of SEQ ID NO: 17; a LCDR2 comprising the amino acid sequence of SEQ ID NO: 18; and a LCDR3 comprising the amino acid sequence of SEQ ID NO: 19.

In some embodiments, the antibody that specifically binds to human DC-SIGN comprises a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; a LCDR1 comprising the amino acid sequence of SEQ ID NO: 20; a LCDR2 comprising the amino acid sequence of SEQ ID NO: 18; and a LCDR3 comprising the amino acid sequence of SEQ ID NO: 16.

In some embodiments, the antibody that specifically binds to human DC-SIGN comprises a HCDR1 comprising the amino acid sequence of SEQ ID NO: 25; a HCDR2 comprising the amino acid sequence of SEQ ID NO: 26; a HCDR3 comprising the amino acid sequence of SEQ ID NO: 27; a LCDR1 comprising the amino acid sequence of SEQ ID NO: 38; a LCDR2 comprising the amino acid sequence of SEQ ID NO: 39; and a LCDR3 comprising the amino acid sequence of SEQ ID NO: 40.

In some embodiments, the antibody that specifically binds to human DC-SIGN comprises a HCDR1 comprising the amino acid sequence of SEQ ID NO: 28; a HCDR2 comprising the amino acid sequence of SEQ ID NO: 26; a HCDR3 comprising the amino acid sequence of SEQ ID NO: 27; a LCDR1 comprising the amino acid sequence of SEQ ID NO: 38; a LCDR2 comprising the amino acid sequence of SEQ ID NO: 39; and a LCDR3 comprising the amino acid sequence of SEQ ID NO: 40.

In some embodiments, the antibody that specifically binds to human DC-SIGN comprises a HCDR1 comprising the amino acid sequence of SEQ ID NO: 29; a HCDR2 comprising the amino acid sequence of SEQ ID NO: 30; a HCDR3 comprising the amino acid sequence of SEQ ID NO: 27; a LCDR1 comprising the amino acid sequence of SEQ ID NO: 41; a LCDR2 comprising the amino acid sequence of SEQ ID NO: 42; and a LCDR3 comprising the amino acid sequence of SEQ ID NO: 43.

In some embodiments, the antibody that specifically binds to human DC-SIGN comprises a HCDR1 comprising the amino acid sequence of SEQ ID NO: 31; a HCDR2 comprising the amino acid sequence of SEQ ID NO: 32; a HCDR3 comprising the amino acid sequence of SEQ ID NO: 33; a LCDR1 comprising the amino acid sequence of SEQ ID NO: 44; a LCDR2 comprising the amino acid sequence of SEQ ID NO: 42; and a LCDR3 comprising the amino acid sequence of SEQ ID NO: 40.

In some embodiments, the antibody that specifically binds to human DC-SIGN comprises a HCDR1 comprising the amino acid sequence of SEQ ID NO: 49; a HCDR2 comprising the amino acid sequence of SEQ ID NO: 26; a HCDR3 comprising the amino acid sequence of SEQ ID NO: 50; a LCDR1 comprising the amino acid sequence of SEQ ID NO: 59; a LCDR2 comprising the amino acid sequence of SEQ ID NO: 39; and a LCDR3 comprising the amino acid sequence of SEQ ID NO: 60.

In some embodiments, the antibody that specifically binds to human DC-SIGN comprises a HCDR1 comprising the amino acid sequence of SEQ ID NO: 51; a HCDR2 comprising the amino acid sequence of SEQ ID NO: 26; a HCDR3 comprising the amino acid sequence of SEQ ID NO: 50; a LCDR1 comprising the amino acid sequence of SEQ ID NO: 59; a LCDR2 comprising the amino acid sequence of SEQ ID NO: 39; and a LCDR3 comprising the amino acid sequence of SEQ ID NO: 60.

In some embodiments, the antibody that specifically binds to human DC-SIGN comprises a HCDR1 comprising the amino acid sequence of SEQ ID NO: 52; a HCDR2 comprising the amino acid sequence of SEQ ID NO: 30; a HCDR3 comprising the amino acid sequence of SEQ ID NO: 50; a LCDR1 comprising the amino acid sequence of SEQ ID NO: 61; a LCDR2 comprising the amino acid sequence of SEQ ID NO: 42; and a LCDR3 comprising the amino acid sequence of SEQ ID NO: 62.

In some embodiments, the antibody that specifically binds to human DC-SIGN comprises a HCDR1 comprising the amino acid sequence of SEQ ID NO: 53; a HCDR2 comprising the amino acid sequence of SEQ ID NO: 32; a HCDR3 comprising the amino acid sequence of SEQ ID NO: 54; a LCDR1 comprising the amino acid sequence of SEQ ID NO: 63; a LCDR2 comprising the amino acid sequence of SEQ ID NO: 42; and a LCDR3 comprising the amino acid sequence of SEQ ID NO: 60.

In some embodiments, the antibody that specifically binds to human DC-SIGN comprises a heavy chain variable region comprising the amino acid sequence of SEQ ID NO: 10, and a light chain variable region comprising the amino acid sequence of SEQ ID NO: 21.

In some embodiments, the antibody that specifically binds to human DC-SIGN comprises a heavy chain comprising the amino acid sequence of SEQ ID NO: 34, and a light chain comprising the amino acid sequence of SEQ ID NO: 45.

In some embodiments, the antibody that specifically binds to human DC-SIGN comprises a heavy chain comprising the amino acid sequence of SEQ ID NO: 55, and a light chain comprising the amino acid sequence of SEQ ID NO: 64.

In some embodiments, the antibody that specifically binds to human DC-SIGN comprises a heavy chain comprising the amino acid sequence of SEQ ID NO: 34, and a light chain comprising the amino acid sequence of SEQ ID NO: 70.

In some embodiments, the antibody that specifically binds to human DC-SIGN comprises a heavy chain comprising the amino acid sequence of SEQ ID NO: 78, and a light chain comprising the amino acid sequence of SEQ ID NO: 84.

In some embodiments, the antibody that specifically binds to human DC-SIGN comprises a heavy chain comprising the amino acid sequence of SEQ ID NO: 90, and a light chain comprising the amino acid sequence of SEQ ID NO: 99.

In some embodiments, the antibody that specifically binds to human DC-SIGN comprises a heavy chain comprising the amino acid sequence of SEQ ID NO: 103, and a light chain comprising the amino acid sequence of SEQ ID NO: 107.

In some embodiments, the antibody that specifically binds to human DC-SIGN comprises a heavy chain comprising the amino acid sequence of SEQ ID NO: 114, and a light chain comprising the amino acid sequence of SEQ ID NO: 120.

In some embodiments, the antibody that specifically binds to human DC-SIGN comprises a heavy chain comprising the amino acid sequence of SEQ ID NO: 55, and a light chain comprising the amino acid sequence of SEQ ID NO: 126.

In some embodiments, the antibody that specifically binds to human DC-SIGN comprises a heavy chain comprising the amino acid sequence of SEQ ID NO: 78, and a light chain comprising the amino acid sequence of SEQ ID NO: 130.

In some embodiments, the antibody that specifically binds to human DC-SIGN comprises a heavy chain comprising the amino acid sequence of SEQ ID NO: 90, and a light chain comprising the amino acid sequence of SEQ ID NO: 134.

In some embodiments, the antibody that specifically binds to human DC-SIGN comprises a heavy chain comprising the amino acid sequence of SEQ ID NO: 145, and a light chain comprising the amino acid sequence of SEQ ID NO: 149.

In some embodiments, the antibody that specifically binds to human DC-SIGN comprises a heavy chain comprising the amino acid sequence of SEQ ID NO: 162, and a light chain comprising the amino acid sequence of SEQ ID NO: 174.

In some embodiments, the antibody that specifically binds to human DC-SIGN comprises a heavy chain comprising the amino acid sequence of SEQ ID NO: 187, and a light chain comprising the amino acid sequence of SEQ ID NO: 199.

In some embodiments, the antibody that specifically binds to human DC-SIGN comprises a heavy chain comprising the amino acid sequence of SEQ ID NO: 212, and a light chain comprising the amino acid sequence of SEQ ID NO: 223.

In some embodiments, the antibody that specifically binds to human DC-SIGN comprises a heavy chain comprising the amino acid sequence of SEQ ID NO: 234, and a light chain comprising the amino acid sequence of SEQ ID NO: 240.

In some embodiments, the antibody that specifically binds to human DC-SIGN comprises a heavy chain comprising the amino acid sequence of SEQ ID NO: 249, and a light chain comprising the amino acid sequence of SEQ ID NO: 260.

In some embodiments, the antibody that specifically binds to human DC-SIGN comprises a heavy chain comprising the amino acid sequence of SEQ ID NO: 273, and a light chain comprising the amino acid sequence of SEQ ID NO: 284.

In some embodiments, the antibody that specifically binds to human DC-SIGN comprises a heavy chain comprising the amino acid sequence of SEQ ID NO: 288, and a light chain comprising the amino acid sequence of SEQ ID NO: 292.

In some embodiments, the antibody that specifically binds to human DC-SIGN comprises a heavy chain comprising the amino acid sequence of SEQ ID NO: 298, and a light chain comprising the amino acid sequence of SEQ ID NO: 284.

In some embodiments, the present disclosure provides antibodies or antibody fragments (e.g., antigen binding fragments) that specifically bind an epitope in human DC-SIGN. In some embodiments, the present disclosure provides antibodies or antibody fragments (e.g., antigen binding fragments) that specifically bind to an epitope in human DC-SIGN, wherein the epitope comprises the amino acid sequence of SEQ ID NOs: 320-323.

In some embodiments, the present disclosure provides antibodies or antibody fragments (e.g., antigen binding fragments) that specifically bind to human DC-SIGN, but not human L-SIGN. For example, the present disclosure provides antibodies or antibody fragments (e.g., antigen binding fragments) that specifically bind to human DC-SIGN at an affinity that is at least 1×, at least 2×, at least 3×, at least 4×, at least 5×, at least 10×, at least 20×, at least 50×, at least 100×, at least 1,000× higher than its affinity to human L-SIGN.

Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

The present invention also provides anti-DC-SIGN antibodies or antigen binding fragments thereof that comprise modifications in the constant regions of the heavy chain, light chain, or both the heavy and light chain wherein particular amino acid residues have mutated to cysteines, also referred to herein at “CysMab” or “Cys” antibodies. As discussed herein, drug moieties may be conjugated site specifically and with control over the number of drug moieties (“DAR Controlled”) to cysteine residues on antibodies. Cysteine modifications to antibodies for the purposes of site specifically controlling immunoconjugation are disclosed, for example, in WO2014/124316, which is incorporated herein by reference in its entirety.

In some embodiments, the anti-DC-SIGN antibodies have been modified at positions 152 and/or 375 of the heavy chain, wherein the positions are defined according to the EU numbering system. Namely, the modifications are E152C and/or S375C. In some embodiments, the anti-DC-SIGN antibodies have been modified at position 152 of the heavy chain, wherein the positions are defined according to the EU numbering system. Namely, the modification is E152C. In some embodiments, the anti-DC-SIGN antibodies have been modified at position 375 of the heavy chain, wherein the positions are defined according to the EU numbering system. Namely, the modification is S375C. In other embodiments, the anti-DC-SIGN antibodies have been modified at position 360 of the heavy chain and position 107 of the kappa light chain, wherein the positions are defined according to the EU numbering system. Namely, the modifications are K360C and K107C.

Identification of Epitopes and Antibodies that Bind to the Same Epitope

The present invention also provides antibodies and antibody fragments (e.g., antigen binding fragments) that specifically bind to the same epitope as the anti-DC-SIGN antibodies described in Table 1, or cross compete with the antibodies described in Table 1. Additional antibodies and antibody fragments (e.g., antigen binding fragments) can therefore be identified based on their ability to cross-compete (e.g., to competitively inhibit the binding of, in a statistically significant manner) with other antibodies of the invention in DC-SIGN binding assays, for example, via BIACORE or assays known to persons skilled in the art for measuring binding. The ability of a test antibody to inhibit the binding of antibodies and antibody fragments (e.g., antigen binding fragments) of the present invention to a DC-SIGN (e.g., human DC-SIGN) demonstrates that the test antibody can compete with that antibody or antibody fragment (e.g., antigen binding fragments) for binding to DC-SIGN; such an antibody may, according to non-limiting theory, bind to the same or a related (e.g., a structurally similar or spatially proximal or overlapping) epitope on the DC-SIGN protein as the antibody or antibody fragment (e.g., antigen binding fragments) with which it competes. In certain embodiments, the antibodies that bind to the same epitope on DC-SIGN as the antibodies or antibody fragments (e.g., antigen binding fragments) described in Table 1 are human or humanized monoclonal antibodies. Such human or humanized monoclonal antibodies can be prepared and isolated as described herein.

### Modification of Framework or Fc Region

Antibodies and antibody conjugates disclosed herein may comprise modified antibodies or antigen binding fragments thereof that comprise modifications to framework residues within VH and/or VL, e.g. to improve the properties of the antibody/antibody conjugate.

In some embodiments, framework modifications are made to decrease immunogenicity of an antibody. For example, one approach is to “back-mutate” one or more framework residues to a corresponding germline sequence. Such residues can be identified by comparing antibody framework sequences to germline sequences from which the antibody is derived. To “match” framework region sequences to desired germline configuration, residues can be “back-mutated” to a corresponding germline sequence by, for example, site-directed mutagenesis. Such “back-mutated” antibodies are also intended to be encompassed by the invention.

Another type of framework modification involves mutating one or more residues within a framework region, or even within one or more CDR regions, to remove T-cell epitopes to thereby reduce potential immunogenicity of the antibody. This approach is also referred to as “deimmunization” and is described in further detail in U.S. Patent Publication No. 20030153043 by Carr et al.

In addition or alternative to modifications made within a framework or CDR regions, antibodies disclosed herein may be engineered to include modifications within the Fc region, typically to alter one or more functional properties of the antibody, such as serum half-life, complement fixation, Fc receptor binding, and/or antigen-dependent cellular cytotoxicity.

Furthermore, an antibody disclosed herein may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In some embodiments antibodies or antibody fragments (e.g., antigen binding fragment) useful in antibody conjugates disclosed herein include modified or engineered antibodies, such as an antibody modified to introduce one or more cysteine residues as sites for conjugation to a drug moiety (Junutula J R, et al.: Nat Biotechnol 2008, 26:925-932). In one embodiment, the invention provides a modified antibody or antibody fragment thereof comprising a substitution of one or more amino acids with cysteine at the positions described herein. Sites for cysteine substitution are in the constant regions of the antibody and are thus applicable to a variety of antibodies, and the sites are selected to provide stable and homogeneous conjugates. A modified antibody or fragment can have two or more cysteine substitutions, and these substitutions can be used in combination with other antibody modification and conjugation methods as described herein. Methods for inserting cysteine at specific locations of an antibody are known in the art, see, e.g., Lyons et al, (1990) Protein Eng., 3:703-708, WO 2011/005481, WO2014/124316, WO 2015/138615. In certain embodiments a modified antibody or antibody fragment comprises a substitution of one or more amino acids with cysteine on its constant region selected from positions 117, 119, 121, 124, 139, 152, 153, 155, 157, 164, 169, 171, 174, 189, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody or antibody fragment, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain.

In particular embodiments a modified antibody or antibody fragment thereof comprises a combination of substitution of two amino acids with cysteine on its constant regions, wherein the modified antibody or antibody fragment thereof comprises cysteines at positions 152 and 375 of an antibody heavy chain, wherein the positions are numbered according to the EU system.

In other particular embodiments a modified antibody or antibody fragment thereof comprises a substitution of one amino acid with cysteine at position 360 of an antibody heavy chain and wherein the positions are numbered according to the EU system.

In other particular embodiments a modified antibody or antibody fragment thereof comprises a substitution of one amino acid with cysteine at position 107 of an antibody light chain and wherein the positions are numbered according to the EU system, and wherein the light chain is a kappa chain.

In additional embodiments antibodies or antibody fragments (e.g., antigen binding fragment) useful in antibody conjugates disclosed herein include modified or engineered antibodies, such as an antibody modified to introduce one or more other reactive amino acid (other than cysteine), including Pcl, pyrrolysine, peptide tags (such as S6, A1 and ybbR tags), and non-natural amino acids, in place of at least one amino acid of the native sequence, thus providing a reactive site on the antibody or antigen binding fragment for conjugation to a drug moiety of Formula (I) or subformulae thereof. For example, the antibodies or antibody fragments can be modified to incorporate Pcl or pyrrolysine (W. Ou et al. (2011) PNAS 108 (26), 10437-10442; WO2014124258) or unnatural amino acids (J. Y. Axup, et al. Proc Natl Acad Sci USA, 109 (2012), pp. 16101-16106; for review, see C. C. Liu and P. G. Schultz (2010) Annu Rev Biochem 79, 413-444; C. H. Kim, et al., (2013) Curr Opin Chem Biol. 17, 412-419) as sites for conjugation to a drug. Similarly, peptide tags for enzymatic conjugation methods can be introduced into an antibody (Strop P. et al. Chem Biol. 2013, 20(2):161-7; Rabuka D., Curr Opin Chem Biol. 2010 December; 14(6):790-6; Rabuka D, et al., Nat Protoc. 2012, 7(6):1052-67). One other example is the use of 4′-phosphopantetheinyl transferases (PPTase) for the conjugation of Coenzyme A analogs (WO2013184514). Methods for conjugating such modified or engineered antibodies with payloads or linker-payload combinations are known in the art.

In another embodiment, an Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl Protein A (SpA) binding relative to native Fc-hinge domain SpA binding. This approach is described in further detail in U.S. Pat. No. 6,165,745 by Ward et al.

In yet other embodiments, an Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in, e.g., U.S. Pat. No. 6,194,551 by Idusogie et al.

In another embodiment, one or more amino acid residues are altered to thereby alter the ability of the antibody to fix complement. This approach is described in, e.g., the PCT Publication WO 94/29351 by Bodmer et al. Allotypic amino acid residues include, but are not limited to, constant region of a heavy chain of the IgG1, IgG2, and IgG3 subclasses as well as constant region of a light chain of the kappa isotype as described by Jefferis et al., MAbs. 1:332-338 (2009).

In a further embodiment, the Fc region is modified to “silence” the effector function of the antibody, for example, reduce or eliminate the ability of the antibody to mediate antibody dependent cellular cytotoxicity (ADCC) and/or antibody dependent cellular phagocytosis (ADCP). This can be achieve, for example, by introducing a mutation in the Fc region of the antibodies. Such mutations have been described in the art: LALA and N297A (Strohl, W., 2009, Curr. Opin. Biotechnol. vol. 20(6):685-691); and D265A (Baudino et al., 2008, J. Immunol. 181: 6664-69; Strohl, W., supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the so-called DAPA mutant comprising D265A and P329A mutations in the IgG1 Fc amino acid sequence. Another silent IgG1 antibody comprises the N297A mutation, which results in aglycosylated/non-glycosylated antibodies.

In yet another embodiment, the Fc region is modified to increase the ability of the antibody to mediate antibody dependent cellular cytotoxicity (ADCC) and/or antibody dependent cellular phagocytosis (ADCP), for example, by modifying one or more amino acid residues to increase the affinity of the antibody for an activating Fcγ receptor, or to decrease the affinity of the antibody for an inhibitory Fcγ receptor. Human activating Fcγ receptors include FcγRIa, FcγRIIa, FcγRIIIa, and FcγRIIIb, and human inhibitory Fcγ receptor includes FcγRIIb. This approach is described in, e.g., the PCT Publication WO 00/42072 by Presta. Moreover, binding sites on human IgG1 for FcγRI, FcγRII, FcγRIII and FcRn have been mapped and variants with improved binding have been described (see Shields et al., J. Biol. Chem. 276:6591-6604, 2001). Optimization of Fc-mediated effector functions of monoclonal antibodies such as increased ADCC/ADCP function has been described (see Strohl, W. R., Current Opinion in Biotechnology 2009; 20:685-691.) In some embodiments, an antibody conjugate comprises an immunoglobulin heavy chain comprising a mutation or combination of mutations conferring enhanced ADCC/ADCP function, e.g., one or more mutations selected from G236A, S239D, F243L, P2471, D280H, K290S, R292P, S298A, S298D, S298V, Y300L, V3051, A330L, 1332E, E333A, K334A, A339D, A339Q, A339T, P396L (all positions by EU numbering).

In another embodiment, the Fc region is modified to increase the ability of the antibody to mediate ADCC and/or ADCP, for example, by modifying one or more amino acids to increase the affinity fo the antibody for an activating receptor that would typically not recognize the parent antibody, such as FcαRI. This approach is descried in, e.g., Borrok et al., mAbs. 7(4):743-751. In particular embodiments, an antibody conjugate comprises an immunoglobulin heavy chain comprising a mutation or a fusion of one or more antibody sequences conferring enhanced ADCC and/or ADCP function.

In still another embodiment, glycosylation of an antibody is modified. For example, an aglycosylated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in, e.g., U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

### Bispecific Antibodies

In certain embodiments, the DC-SIGN antibody molecules disclosed herein are in the form of a bispecific or multispecific antibody molecule. In one embodiment, the bispecific antibody molecule has a first binding specificity to DC-SIGN as disclosed herein and a second binding specificity, e.g., a second binding specificity to PD-1, PD-L1, PD-L2, CTLA-4, TIM-3, LAG-3, CEACAM (e.g., CEACAM-1, CEACAM-3, and/or CEACAM-5), VISTA, BTLA, TIGIT, LAIR1, CD160, 2B4, TGFβR, IDO (indoleamine-2,3 dioxygenase), OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83. Any combination of the aforesaid molecules can be made in a multispecific antibody molecule, e.g., a trispecific antibody that includes a first binding specificity to DC-SIGN, and second and third binding specificities to two or more of: PD-1, TIM-3, LAG-3, or PD-L2.

### Production of Anti-DC-SIGN Antibodies

Anti-DC-SIGN antibodies and antibody fragments (e.g., antigen binding fragments) thereof can be produced by any means known in the art, including but not limited to, recombinant expression, chemical synthesis, and enzymatic digestion of antibody tetramers, whereas full length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

Also provided herein are polynucleotides encoding antibodies described herein, e.g., polynucleotides encoding heavy or light chain variable regions or segments comprising complementarity determining regions as described herein. In some embodiments, a polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide of SEQ ID NO: 11, 35, 56, 79, 91, 104, 115, 146, 163, 188, 213, 235, 250, 274, 289, or 299. In some embodiments, a polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide of SEQ ID NO: 22, 46, 65, 71, 85, 100, 108, 121, 127, 131, 135, 150, 175, 200, 224, 241, 261, 285, or 293.

In some embodiments, a polynucleotide encoding the heavy chain has at least 85%, 89%, 90%, 91%, 92%, 93%, 94%, 95%, 96%, 97%, 98%, 99%, or 100% nucleic acid sequence identity with a polynucleotide of any of SEQ ID NOs: 13, 37, 58, 81, 93, 106, 117, 148, 165, 190, 215, 237, 252, 276, 291, or 301. In some embodiments, a polynucleotide encoding the light chain has at least 85%, 89%, 90%, 91%, 92%, 93%, 94%, 95%, 96%, 97%, 98%, 99%, or 100% nucleic acid sequence identity with a polynucleotide of SEQ ID NO: 24, 48, 67, 73, 87, 102, 110, 123, 129, 133, 137, 152, 177, 202, 226, 243, 263, 287, or 295.

Some polynucleotides disclosed herein encode a variable region of an anti-DC-SIGN antibody. Some polynucleotides disclosed herein encode both a variable region and a constant region of an anti-DC-SIGN antibody. Some polynucleotide sequences encode a polypeptide that comprises variable regions of both a heavy chain and a light chain of an anti-DC-SIGN antibody. Some polynucleotides encode two polypeptide segments that respectively are substantially identical to the variable regions of a heavy chain and a light chain of any anti-DC-SIGN antibodies disclosed herein.

Polynucleotide sequences can be produced by de novo solid-phase DNA synthesis or by PCR mutagenesis of an existing sequence (e.g., sequences as described in the Examples below) encoding an anti-DC-SIGN antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided are expression vectors and host cells for producing anti-DC-SIGN antibodies described above. Various expression vectors can be employed to express polynucleotides encoding anti-DC-SIGN antibody chains or binding fragments. Both viral-based and nonviral expression vectors can be used to produce antibodies in a mammalian host cell.

Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of anti-DC-SIGN polynucleotides and polypeptides in mammalian (e.g., human) cells include pThioHis A, B & C, pCDNATM3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Choice of expression vector depends on the intended host cells in which a vector is to be expressed. Typically, expression vectors contain a promoter and other regulatory sequences (e.g., enhancers) that are operably linked to polynucleotides encoding an anti-DC-SIGN antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by host cells. In addition to promoters, other regulatory elements may also be required or desired for efficient expression of an anti-DC-SIGN antibody chain or fragment. Elements typically include an ATG initiation codon and adjacent ribosome binding site or other sequences. In addition, efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, an SV40 enhancer or CMV enhancer may be used to increase expression in mammalian host cells.

Expression vectors may also provide a secretion signal sequence position to form a fusion protein with polypeptides encoded by inserted anti-DC-SIGN antibody sequences. More often, inserted anti-DC-SIGN antibody sequences are linked to a signal sequence before inclusion in the vector. Vectors to be used to receive sequences encoding anti-DC-SIGN antibody light and heavy chain variable domains sometimes also encode constant regions or parts thereof. Such vectors allow expression of variable regions as fusion proteins with constant regions, thereby leading to production of intact antibodies or fragments thereof. Typically, such constant regions are human.

Host cells for harboring and expressing anti-DC-SIGN antibody chains can be either prokaryotic or eukaryotic. E. coli is one prokaryotic host useful for cloning and expressing polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as a lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-DC-SIGN polypeptides disclosed herein. Insect cells in combination with baculovirus vectors can also be used.

In some particular embodiments, mammalian host cells are used to express and produce anti-DC-SIGN polypeptides of the present disclosure. For example, they can be either a hybridoma cell line expressing endogenous immunoglobulin genes (e.g., myeloma hybridoma clones) or a mammalian cell line harboring an exogenous expression vector (e.g., the SP2/0 myeloma cells). These include any normal mortal or normal or abnormal immortal animal or human cell. For example, a number of suitable host cell lines capable of secreting intact immunoglobulins have been developed, including various CHO cell lines, Cos cell lines, HeLa cells, myeloma cell lines, transformed B-cells and hybridomas. Use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. Expression vectors usually contain promoters derived from mammalian genes or from mammalian viruses. Suitable promoters may be constitutive, cell type-specific, stage-specific, and/or modulatable or regulatable. Useful promoters include, but are not limited to, a metallothionein promoter, a constitutive adenovirus major late promoter, a dexamethasoneinducible MMTV promoter, a SV40 promoter, a MRP polIII promoter, a constitutive MPSV promoter, a tetracycline-inducible CMV promoter (such as the human immediate-early CMV promoter), a constitutive CMV promoter, and promoter-enhancer combinations known in the art.

Methods for introducing expression vectors containing polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-DC-SIGN antibody chains or binding fragments can be prepared using expression vectors disclosed herein which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### II) DC-SIGN Antibody Conjugates

The present invention provides antibody drug conjugates, also referred to as conjugates, antibody conjugates, or immunoconjugates, where an antibody, or fragment thereof (e.g. antigen binding fragment), or its functional equivalent that specifically binds to DC-SIGN is linked to a drug moiety. In one aspect, the antibodies, antigen binding fragments, or their functional equivalents of the invention are coupled to a drug moiety (D). In some embodiments, the drug moiety is coupled to the antibody, or fragment thereof, via a linker (L). In some embodiments, the linker may be a non-cleavable or cleavable linker, i.e. the linker comprises cleavage elements.

In some embodiments, the antibody conjugates disclosed herein comprise Formula (III):

Ab-(L-(D)ₘ)ₙ (Formula (III))
wherein:
   Ab is an anti-DC-SIGN antibody or a functional fragment thereof;
   L is a linker;
   D is the drug moiety;
   m is an integer from 1 to 8; and
   n is an integer from 1 to 20.

In some embodiments, the drug moiety D is an anti-cancer agent, an autoimmune treatment agent, an anti-inflammatory agent, an antifungal agent, an antibacterial agent, an anti-parasitic agent, an anti-viral agent, or an anesthetic agent. In some embodiments, the drug moiety D is a targeted inhibitor. In some embodiments, the drug moiety D is a cytotoxic compound. In some embodiments, the drug moiety D is a radiotoxin, including a radioactive isotope, or a radio-labeled nuclide or protein. In some embodiments, the drug moiety D is a heterologous protein or peptide.

In some embodiments, the drug moiety D is an immunomodulatory compound. In some embodiments, the drug moiety D is an immunostimulatory compound, wherein the immunostimulatory compound is not a STING agonist. The antibody drug conjugates of the invention can deliver an effective dose of a drug moiety (e.g., an immunostimulatory molecule, wherein the immunostimulatory molecule is not a STING agonist) to DC-SIGN+ cells, such as dendritic cells (DCs) and/or macrophages. In some embodiments, the antibody drug conjugates of the invention can deliver an effective dose of a drug moiety (e.g., an immunostimulatory molecule, wherein the immunostimulatory molecule is not a STING agonist) to tumor residing antigen presenting cells, such as tumor residing DCs and/or macrophages, which stimulates activation of the DC-SIGN expressing cells and triggers an immune response including tumor-specific T-cell activation, in the tumor. The antibody drug conjugate can also deliver an effective dose of a drug moiety (e.g., an immunostimulatory molecule, wherein the immunostimulatory molecule is not a STING agonist) to lymphoid tissue-resident and peripheral tissue-resident DC-SIGN expressing cells, including dendritic cells and macrophages. Delivery of the antibody drug conjugate to DC-SIGN expressing cells not located in the tumor also stimulates activation of the DC-SIGN expressing cells and triggers an immune response.

In some embodiments, the antibody conjugates of the invention comprise a TLR7 agonist and have the structure of Formula (II):

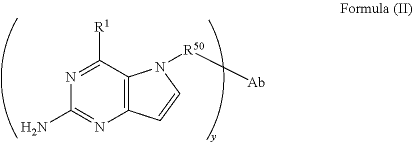

wherein:

### R⁵⁰ is

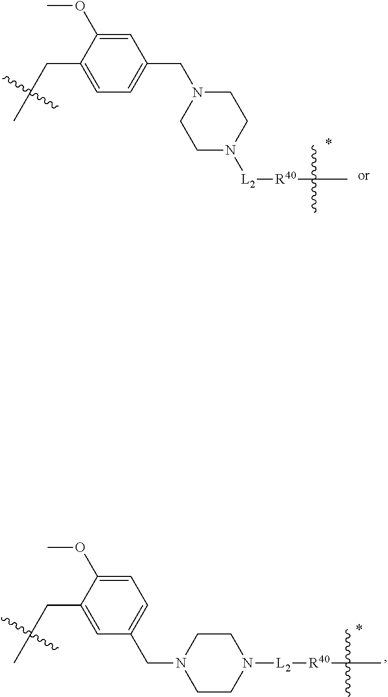

where the * indicates the point of attachment to Ab;
Ab is an anti-DC-SIGN antibody disclosed herein or antigen binding fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴⁰;

### R⁴⁰ is

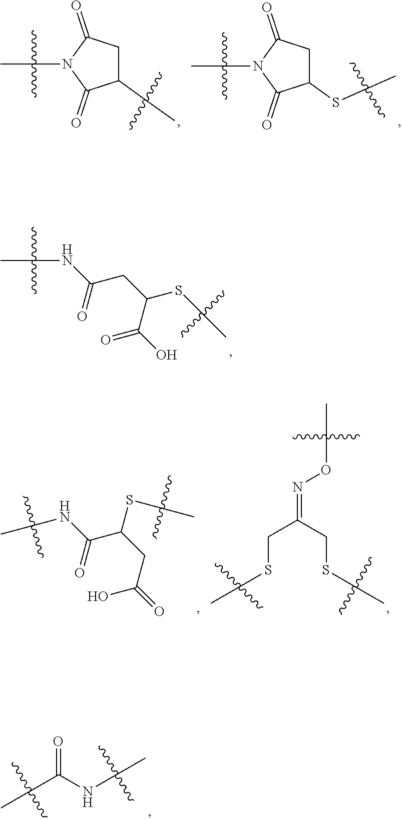 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—,

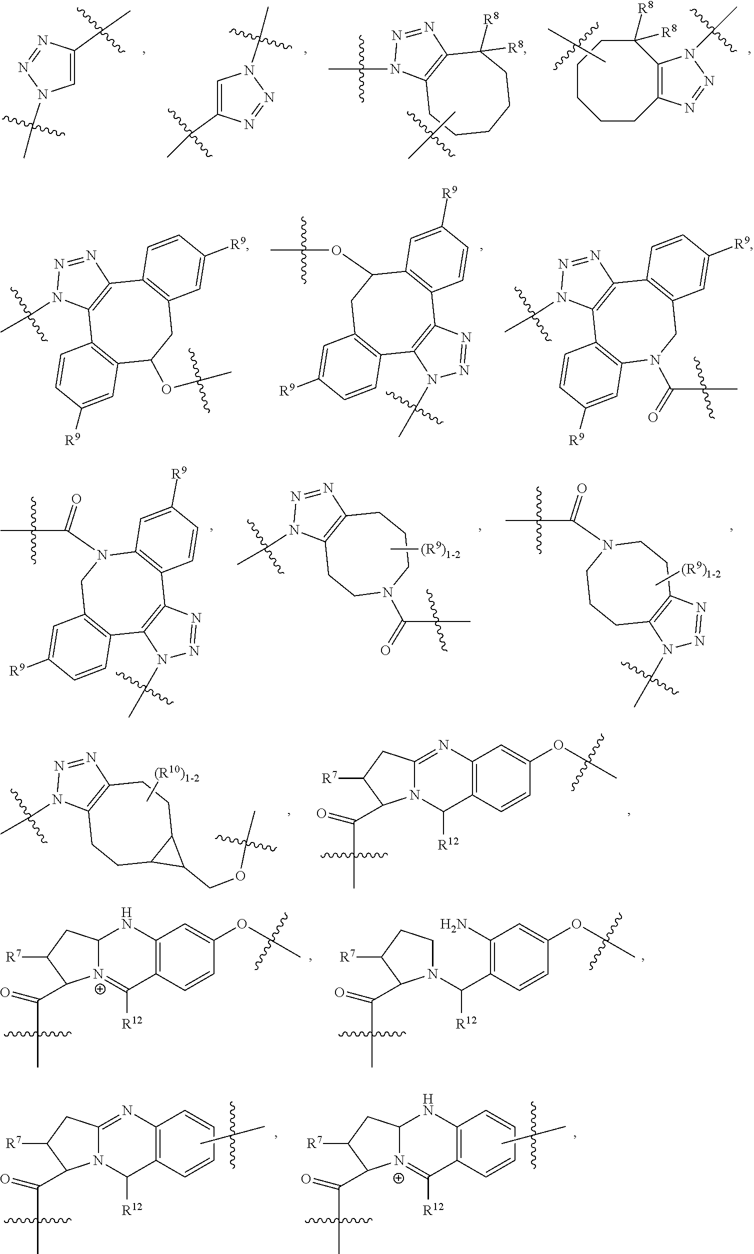

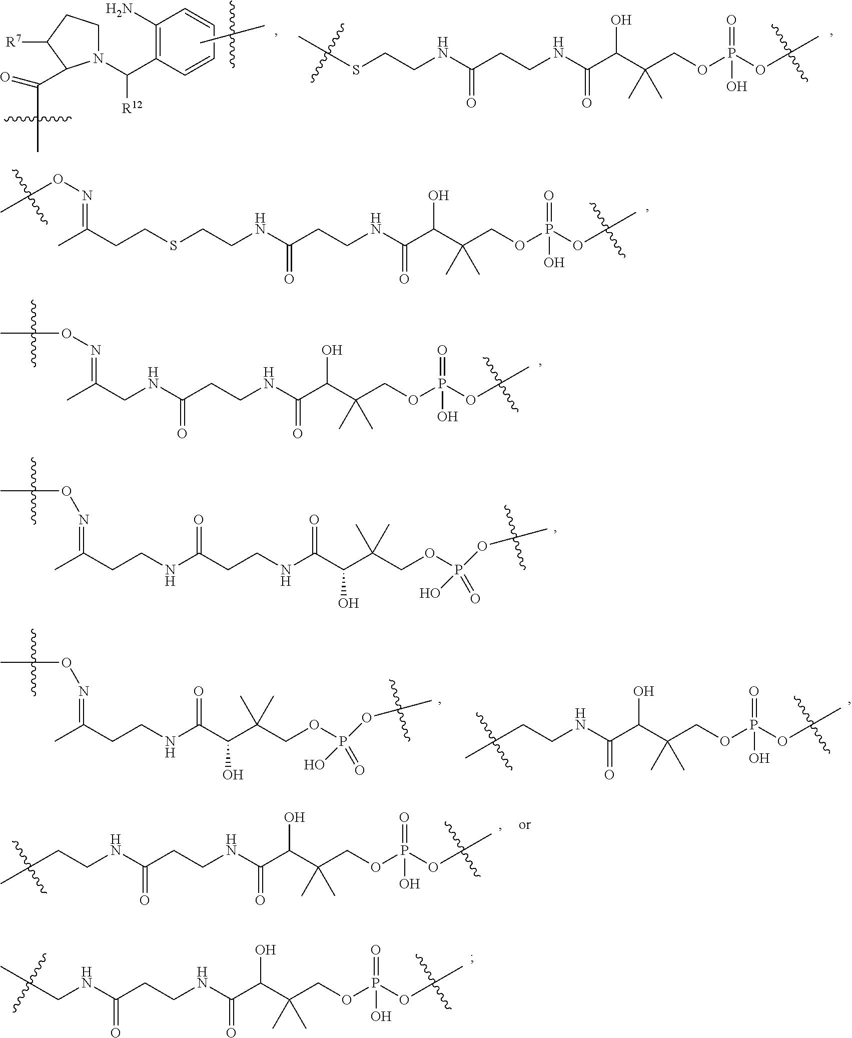

### X₁ is

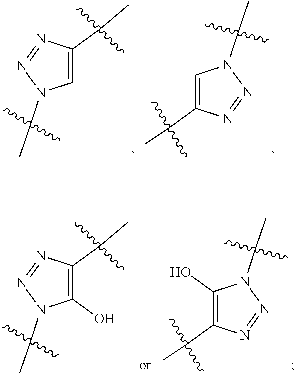 [SMILES: *c1cn(*)nn1 ; *c1cn(*)nn1 ; *c1nnn(*)c1O ; *c1nnn(*)c1O]

### X₂ is

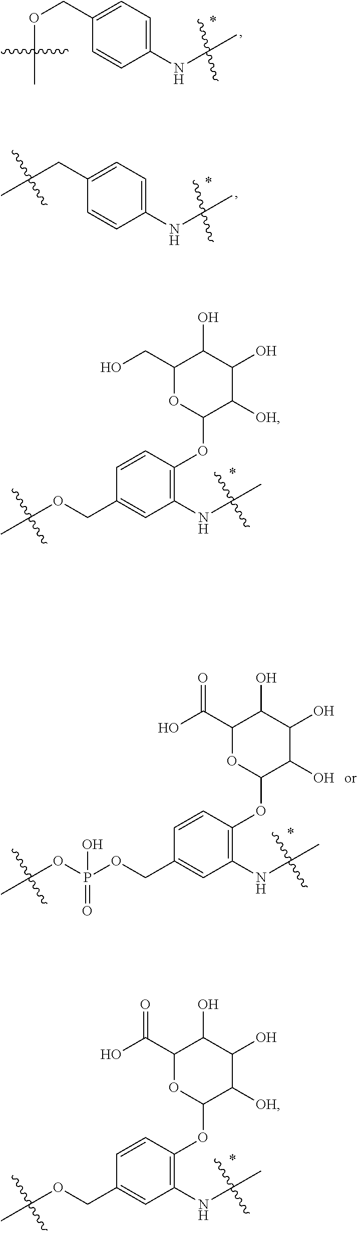 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;

### X₃ is

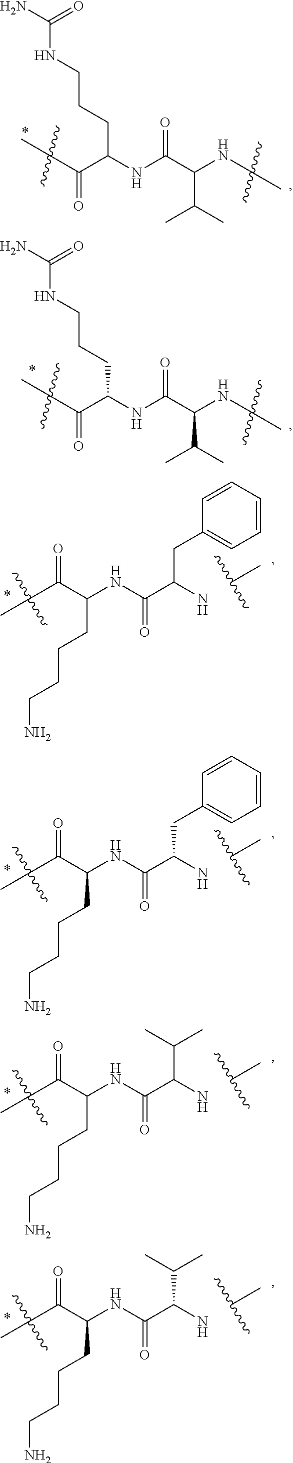

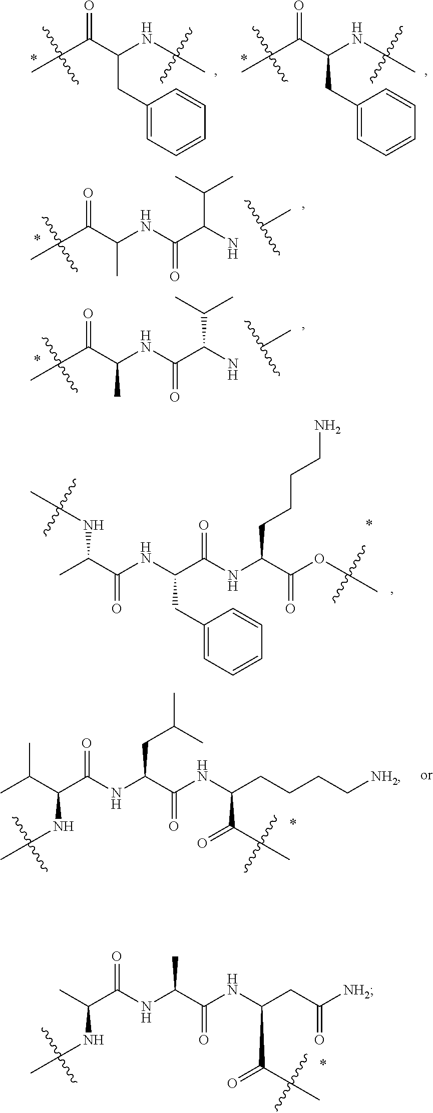

where the *of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Certain aspects and examples of the compounds of Formula (II) are provided in the following listing of additional, enumerated embodiments. It will be recognized that features specified in each embodiment may be combined with other specified features to provide further embodiments of the present invention.

Embodiment 85. The antibody conjugates of Formula (II), wherein:
R⁵⁰ is

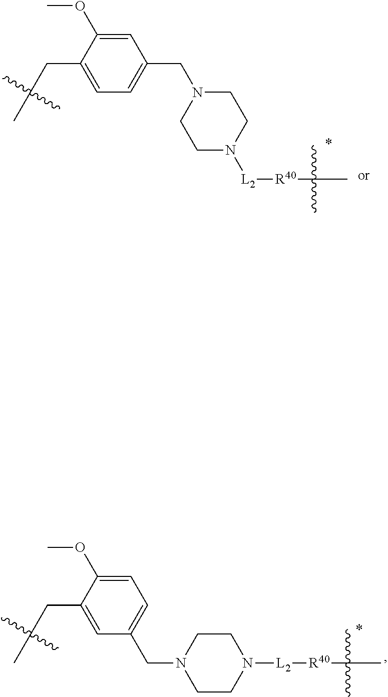

where the * indicates the point of attachment to Ab;
Ab is an anti-DC-SIGN antibody disclosed herein or antigen binding fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴⁰;
R⁴⁰ is

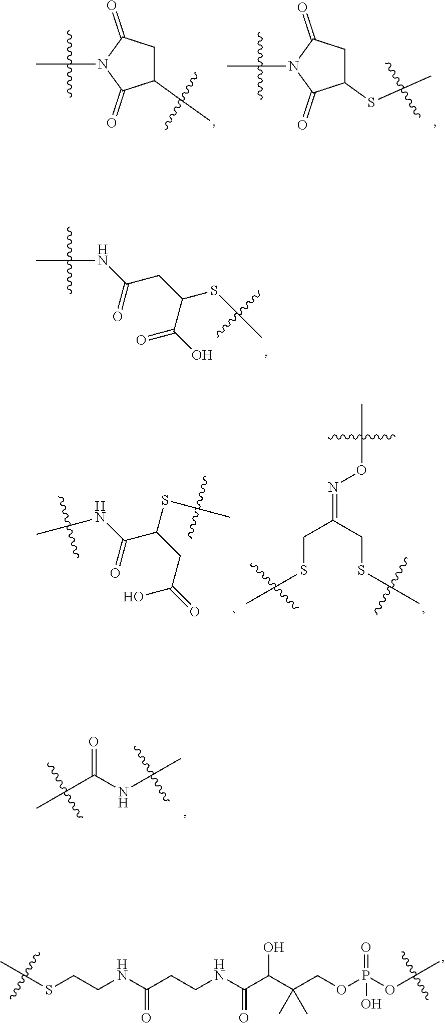

—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—,

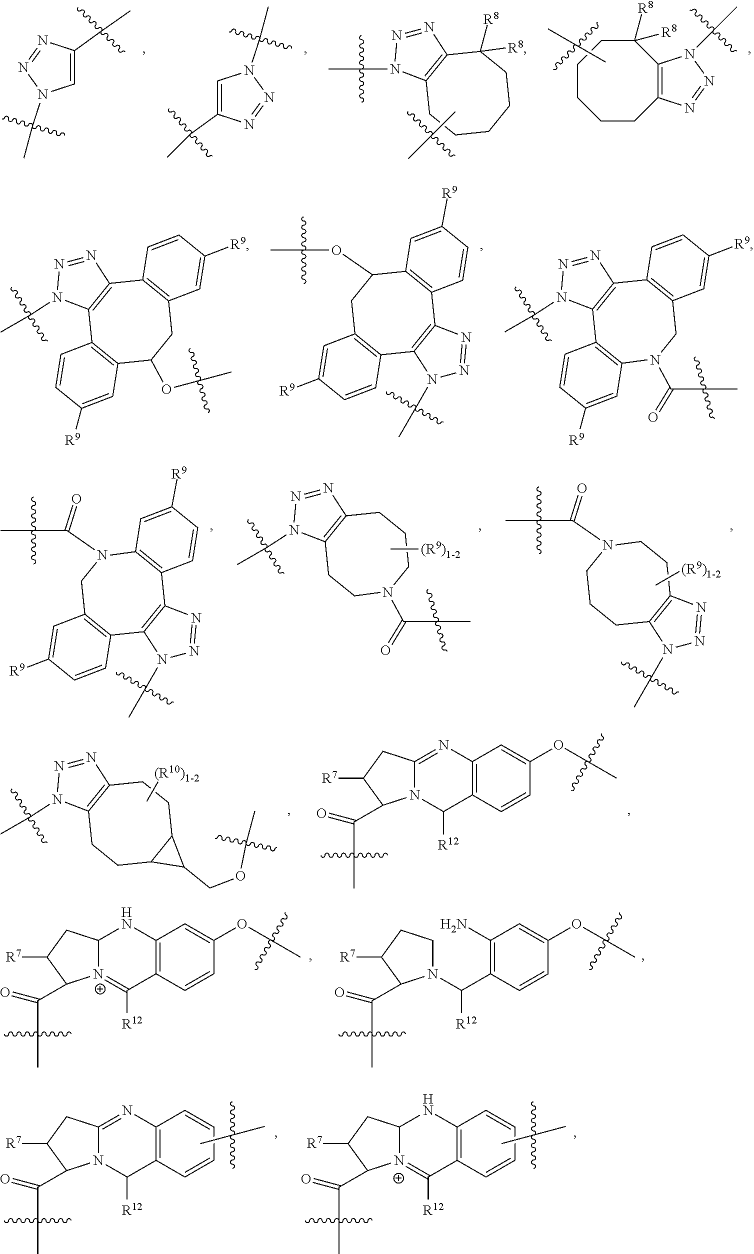

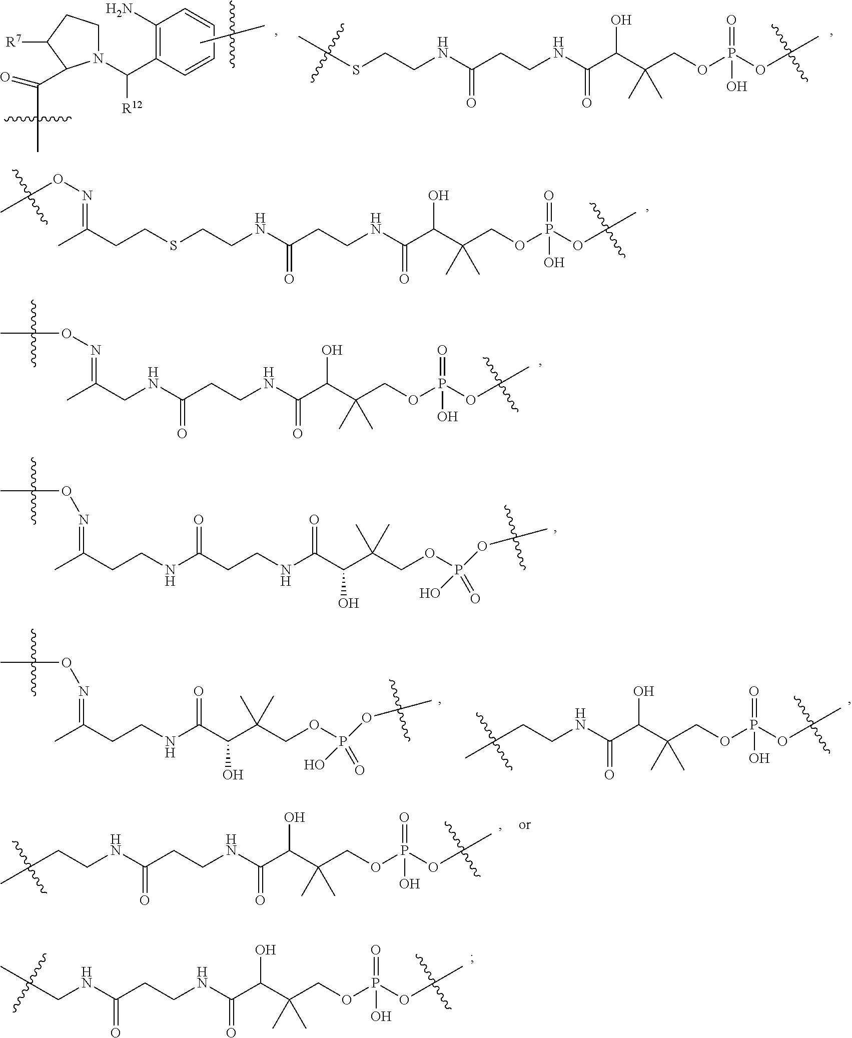

X₁ is

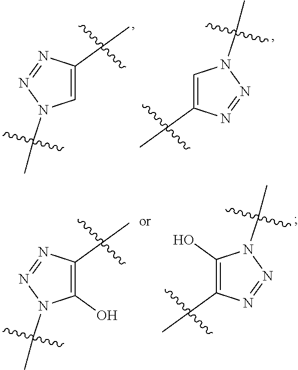 [SMILES: *c1cn(*)nn1 ; *c1cn(*)nn1 ; *c1nnn(*)c1O ; *c1nnn(*)c1O]

X₂ is

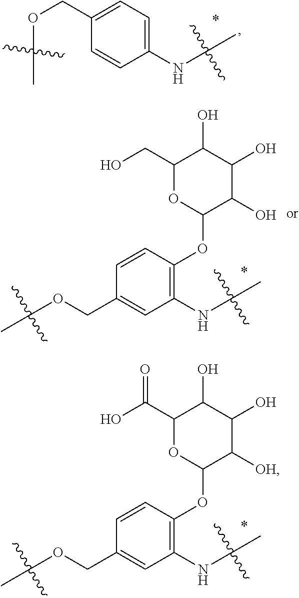 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃;
X₃ is

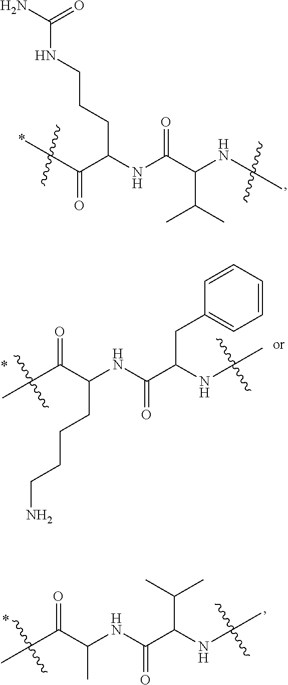

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 86. The antibody conjugate of Formula (II) having the structure of Formula (IIa) or Formula (IIb), and the pharmaceutically acceptable salts thereof:

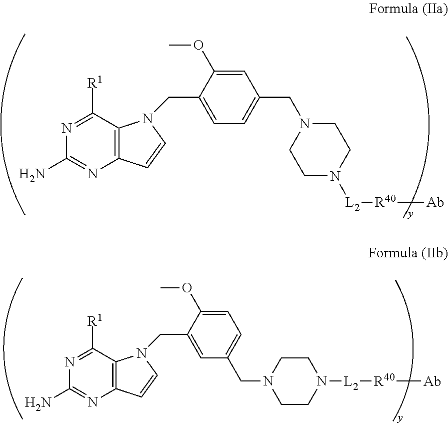

wherein:
Ab is an anti-DC-SIGN antibody disclosed herein or antigen binding fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ**-, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ**-, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴⁰;

### R⁴⁰ is

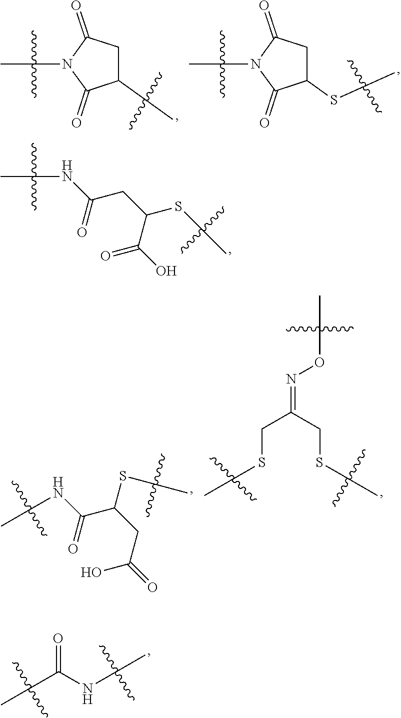 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—,

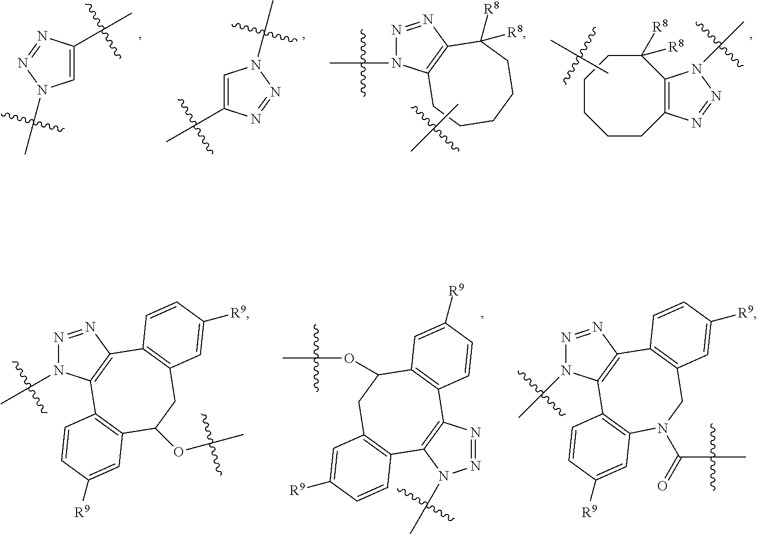

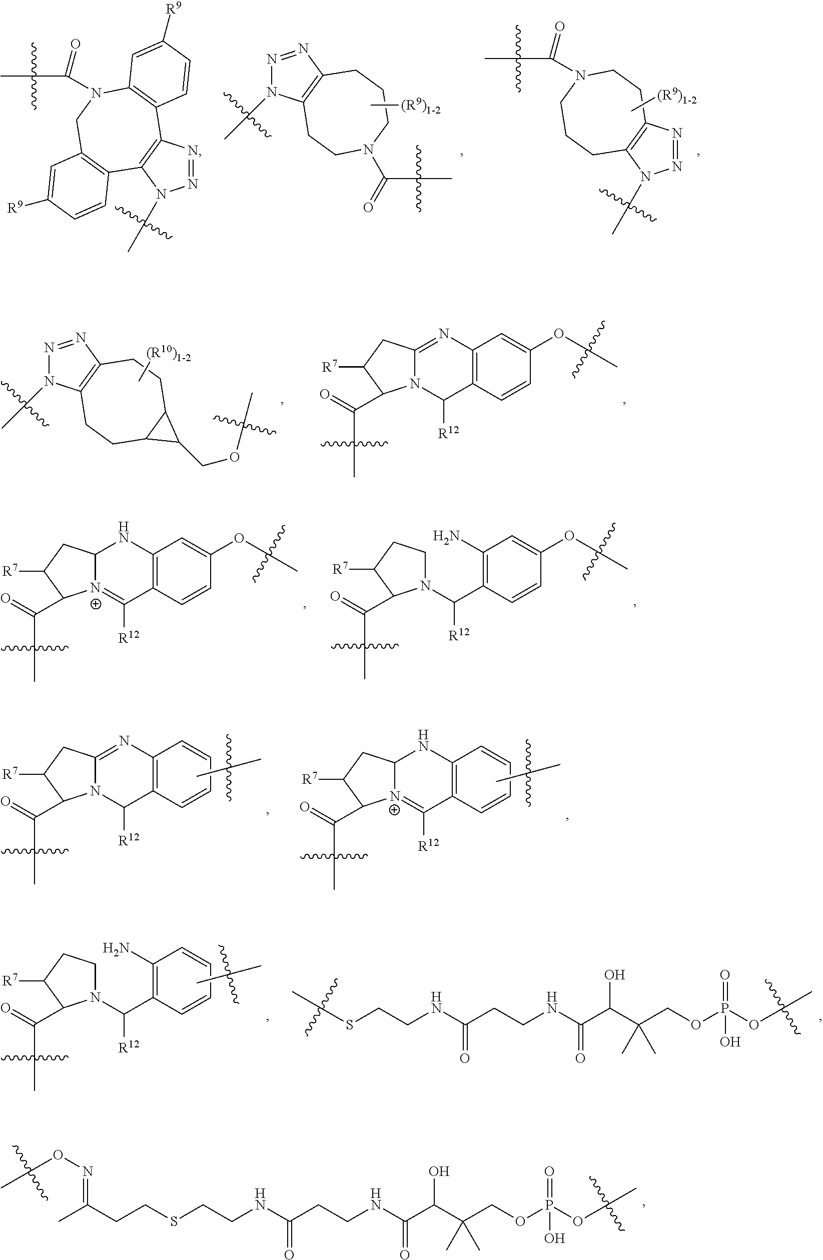

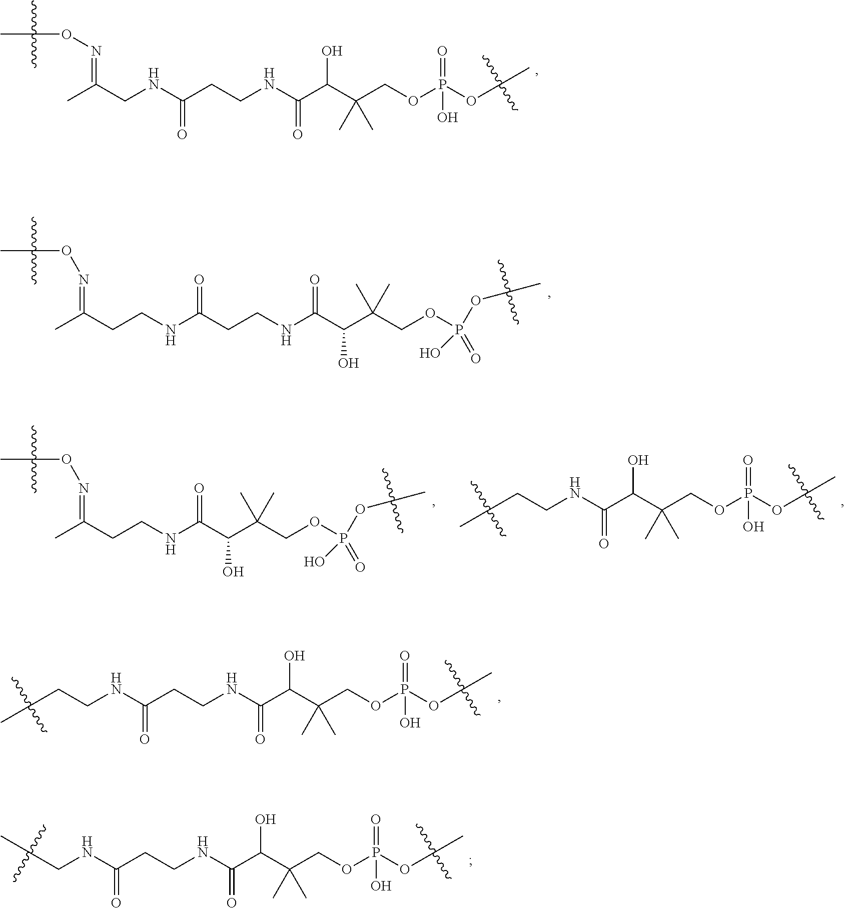 [SMILES: *O/N=C(\C)CNC(=O)CCNC(=O)C(O)C(C)(C)COP(=O)(O)O* ; *O/N=C(\C)CCNC(=O)CCNC(=O)[C@@H](O)C(C)(C)COP(=O)(O)O* ; *O/N=C(\C)CCNC(=O)[C@@H](O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]

### X₁ is

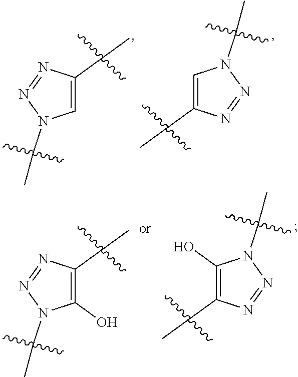 [SMILES: *c1cn(*)nn1 ; *c1cn(*)nn1 ; *c1nnn(*)c1O ; *c1nnn(*)c1O]

### X₂ is

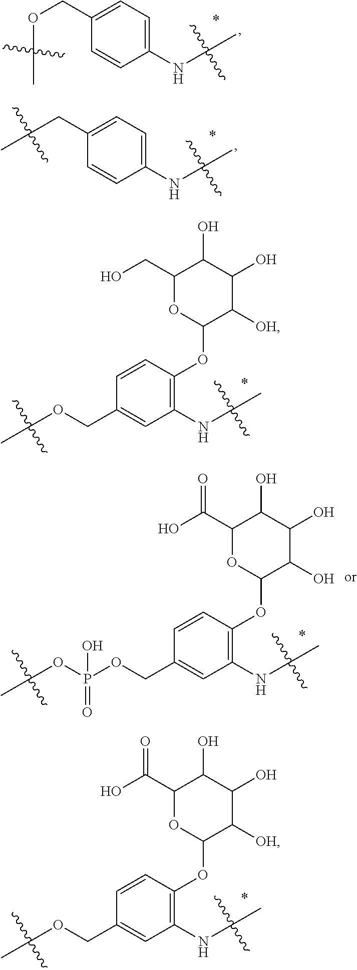 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;

### X₃ is

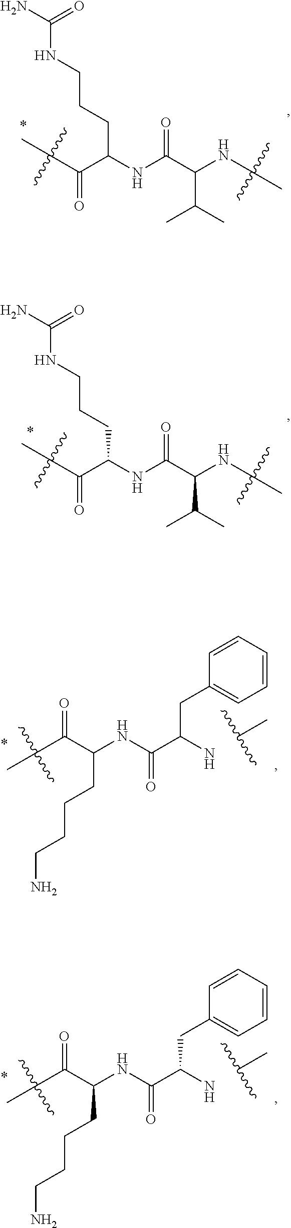

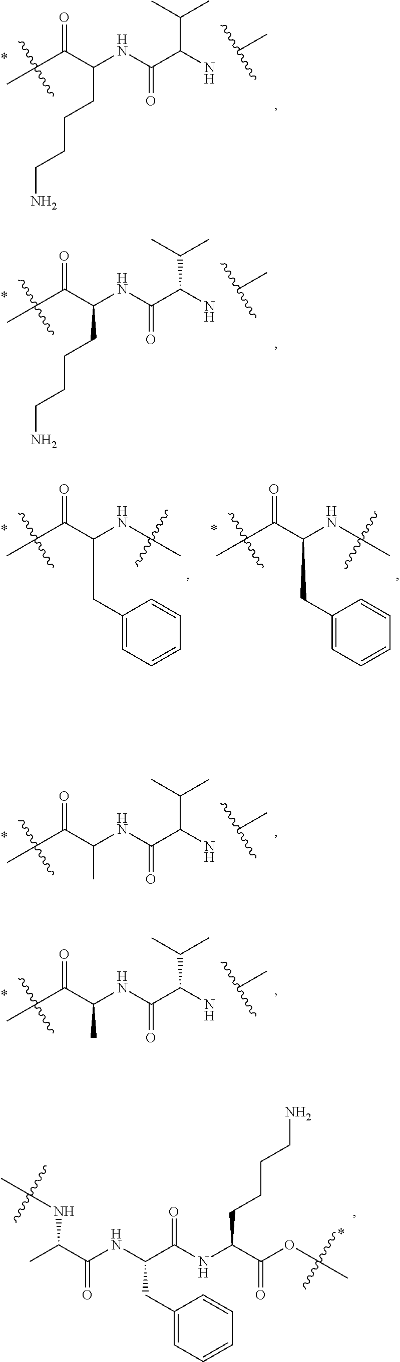

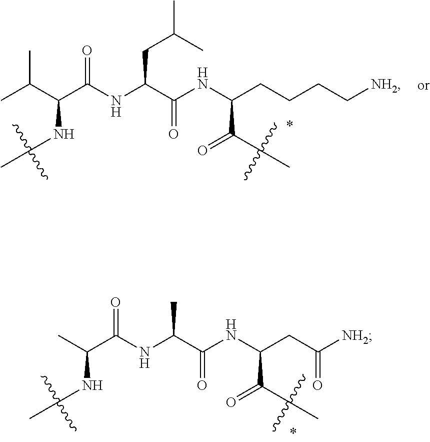 [SMILES: *N[C@H](C(=O)N[C@@H](CC(C)C)C(=O)N[C@@H](CCCCN)C(*)=O)C(C)C ; *N[C@@H](C)C(=O)N[C@@H](C)C(=O)N[C@@H](CC(N)=O)C(*)=O]

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 87. The antibody conjugate of Formula (IIa) or Formula (IIb), and the pharmaceutically acceptable salts thereof, wherein:
Ab is an anti-DC-SIGN antibody disclosed herein or antigen binding fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴⁰;

### R⁴⁰ is

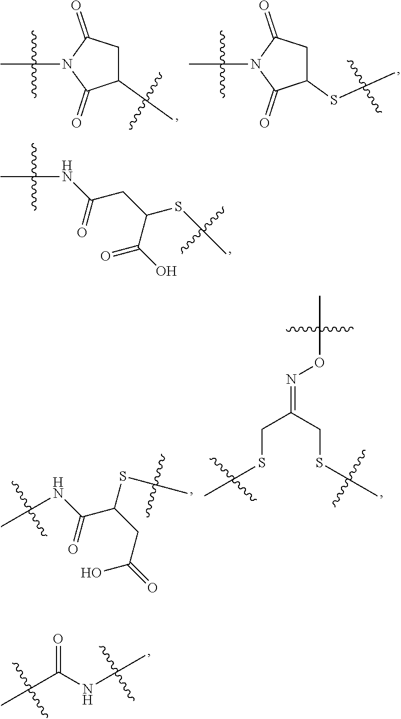 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—,

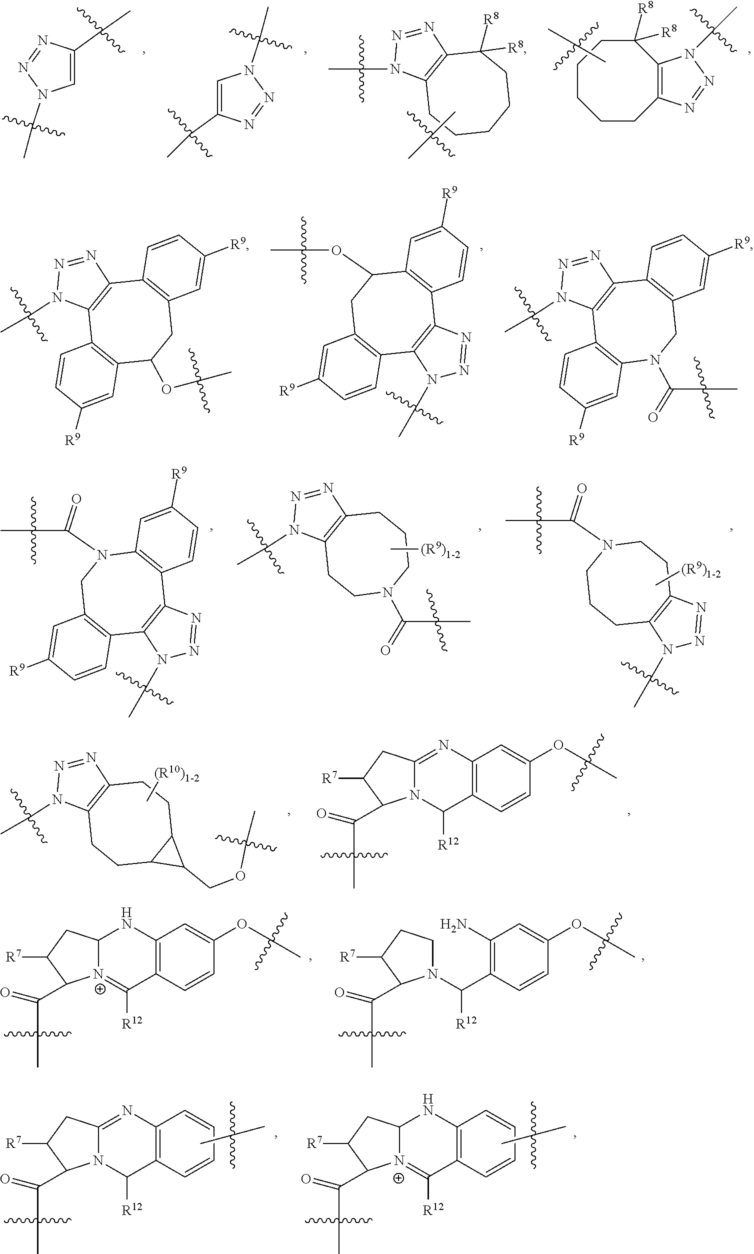

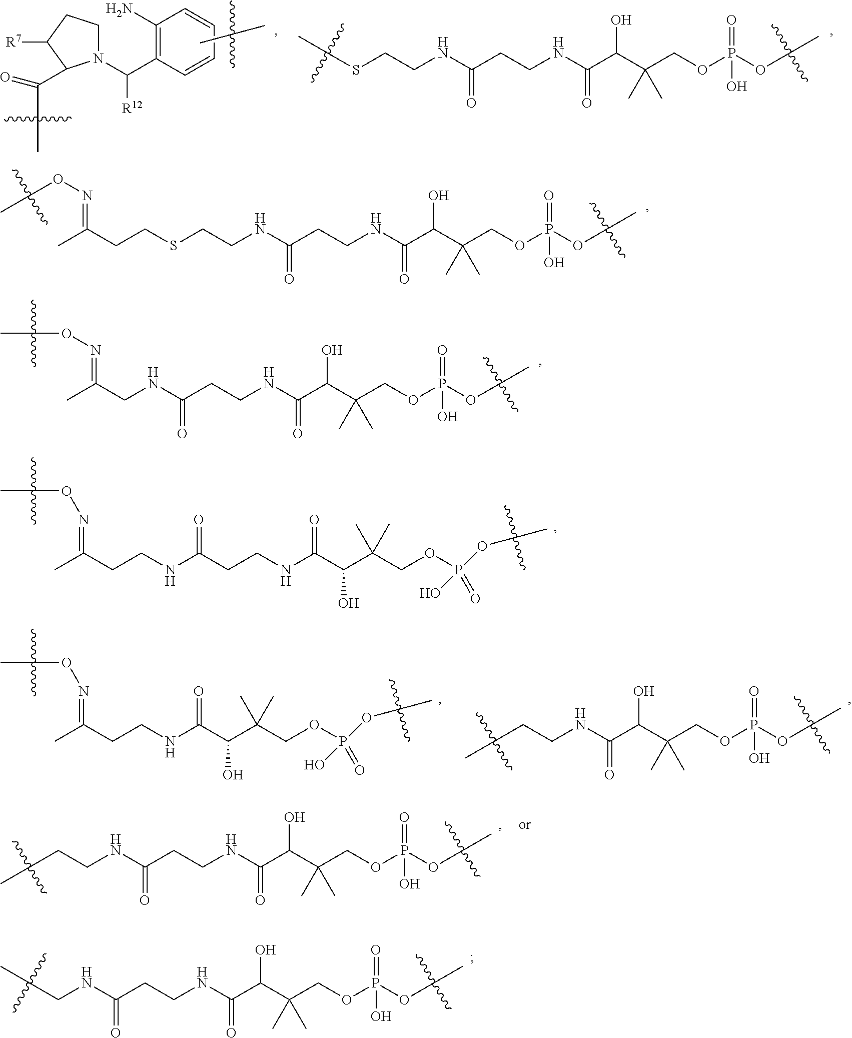

X₁ is

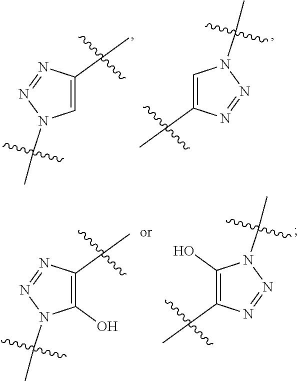 [SMILES: *c1cn(*)nn1 ; *c1cn(*)nn1 ; *c1nnn(*)c1O ; *c1nnn(*)c1O]

X₂ is

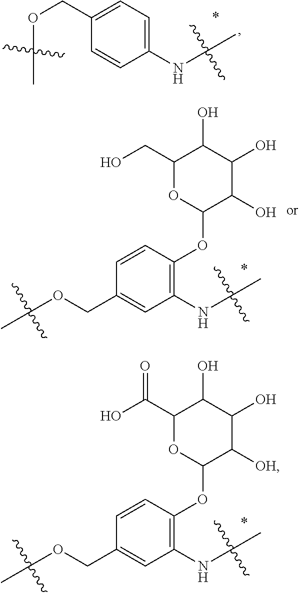 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃;
X₃ is

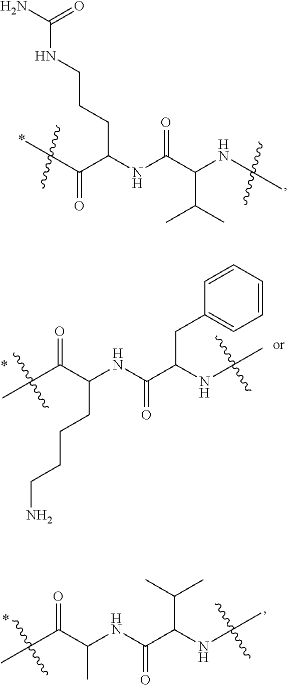

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 88. The antibody conjugate of Formula (II) having the structure of Formula (IIa) or Formula (IIb), and the pharmaceutically acceptable salts thereof:

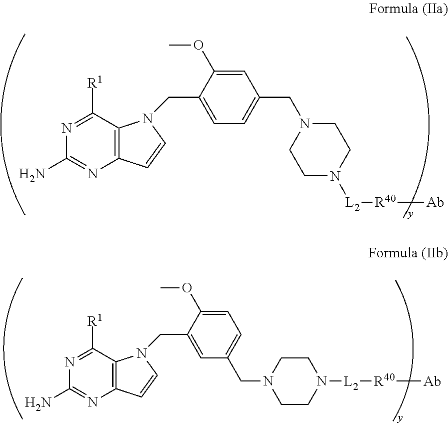

wherein:
Ab is anti-DC-SIGN antibody disclosed herein or antigen binding fragment thereof;
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴⁰;
R⁴⁰ is

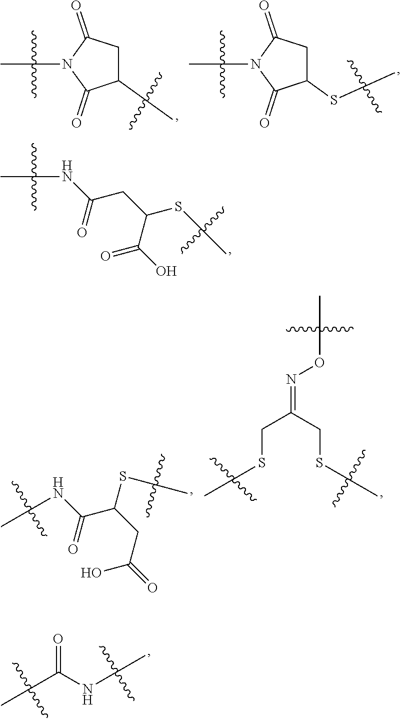 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

### or —S—;

X₁ is

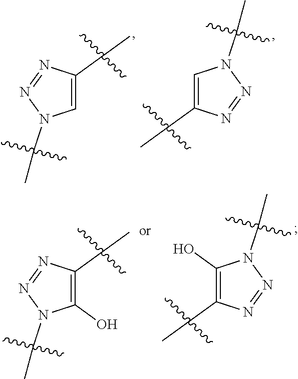 [SMILES: *c1cn(*)nn1 ; *c1cn(*)nn1 ; *c1nnn(*)c1O ; *c1nnn(*)c1O]

X₂ is

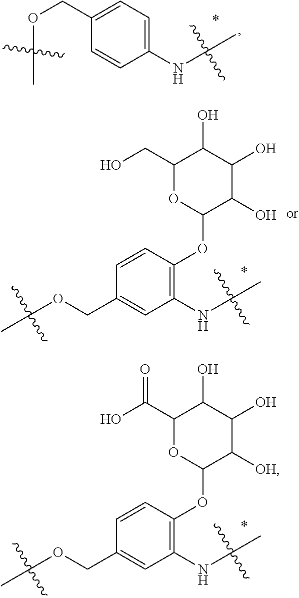 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃;
X₃ is

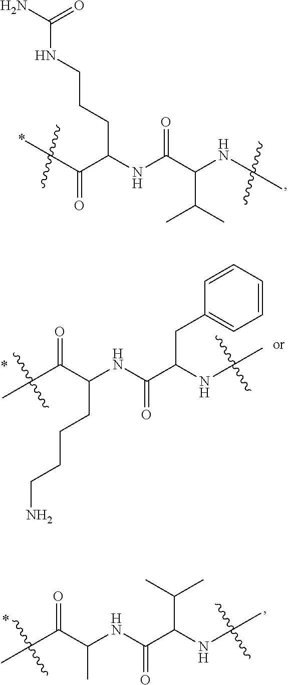

where the * of X₃ indicates the point of attachment to X₂;
each R⁷ is independently selected from H and C₁-C₆alkyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 89. The antibody conjugate of Formula (II), Formula (IIa) or Formula (IIb), wherein:
Ab is an anti-DC-SIGN antibody disclosed herein or antigen binding fragment thereof;
R¹ is —NHR² or —NHCHR²R³;
R² is —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ**-, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴⁰;
R⁴⁰ is

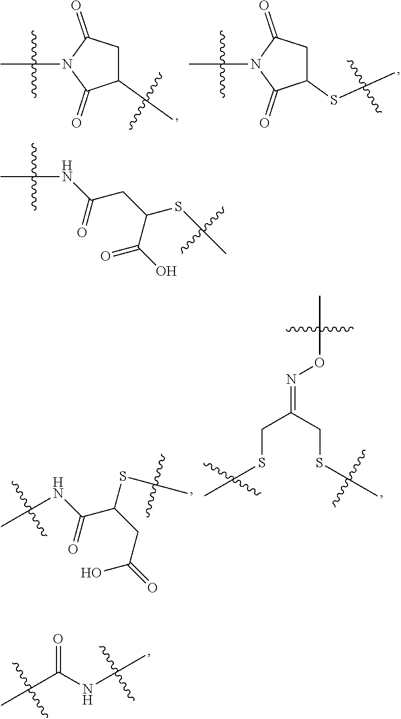 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]

X₁ is

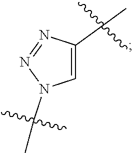 [SMILES: *c1cn(*)nn1]

X₂ is

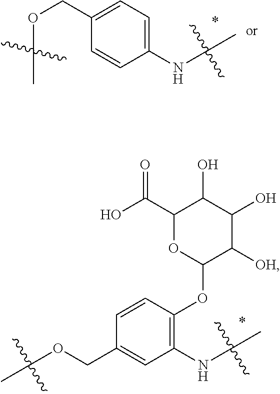 [SMILES: *Nc1ccc(CO*)cc1 ; *Nc1cc(CO*)ccc1OC1OC(C(=O)O)C(O)C(O)C1O]

where the * of X₂ indicates the point of attachment to X₃;
X₃ is

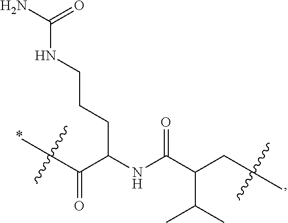 [SMILES: *CC(C(=O)NC(CCCNC(N)=O)C(*)=O)C(C)C]

where the * of X₃ indicates the point of attachment to X₂; each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 90. The antibody conjugate of Formula (II), Formula (IIa) or Formula (IIb), wherein:
Ab is an anti-DC-SIGN antibody disclosed herein or antigen binding fragment thereof;
R¹ is —NHR²;
R² is —C₄-C₆alkyl;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴⁰;
R⁴⁰ is

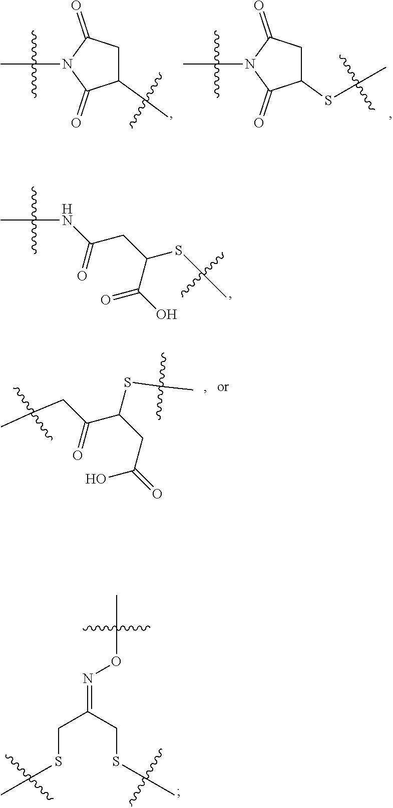 [SMILES: *C1CC(=O)N(*)C1=O ; *SC1CC(=O)N(*)C1=O ; *NC(=O)CC(S*)C(=O)O ; *CC(=O)C(CC(=O)O)S* ; *ON=C(CS*)CS*]

X₁ is

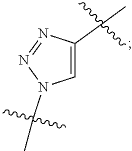 [SMILES: *c1cn(*)nn1]

X₂ is

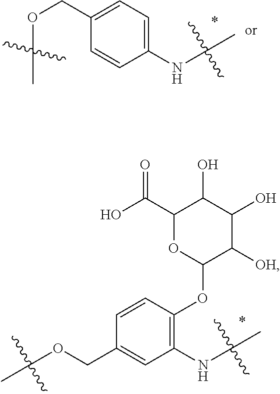 [SMILES: *Nc1ccc(CO*)cc1 ; *Nc1cc(CO*)ccc1OC1OC(C(=O)O)C(O)C(O)C1O]

where the * of X₂ indicates the point of attachment to X₃;
X₃ is

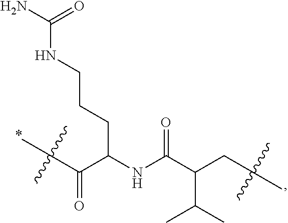 [SMILES: *CC(C(=O)NC(CCCNC(N)=O)C(*)=O)C(C)C]

where the * of X₃ indicates the point of attachment to X₂; each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 91. The antibody conjugate of Formula (II), Formula (IIa) or Formula (IIb), wherein:
Ab is an anti-DC-SIGN antibody disclosed herein or antigen binding fragment thereof;
R¹ is —NHR²;
R² is —C₄-C₆alkyl;
L₂ is —(CH₂)ₙ—, —((CH₂)ₙO)ₜ(CH₂)ₙ—**, —(CH₂)ₙX₁(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—** or —C(═O)X₂C(═O)(CH₂)ₙNHC(═O)(CH₂)ₙ—**, where the ** of L₂ indicates the point of attachment to R⁴⁰;
R⁴⁰ is

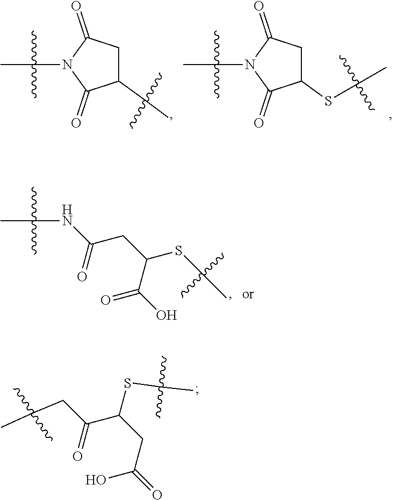 [SMILES: *C1CC(=O)N(*)C1=O ; *SC1CC(=O)N(*)C1=O ; *NC(=O)CC(S*)C(=O)O ; *CC(=O)C(CC(=O)O)S*]

X₁ is

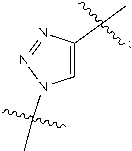 [SMILES: *c1cn(*)nn1]

X₂ is

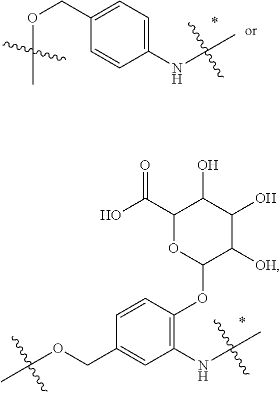 [SMILES: *Nc1ccc(CO*)cc1 ; *Nc1cc(CO*)ccc1OC1OC(C(=O)O)C(O)C(O)C1O]

where the * of X₂ indicates the point of attachment to X₃;
X₃ is

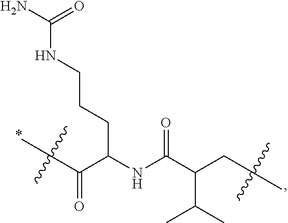 [SMILES: *CC(C(=O)NC(CCCNC(N)=O)C(*)=O)C(C)C]

where the * of X₃ indicates the point of attachment to X₂; each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 92. The antibody conjugate of Formula (II), Formula (IIa) or Formula (IIb), wherein:
Ab is an anti-DC-SIGN antibody disclosed herein or antigen binding fragment thereof;
R¹ is —NHR²;
R² is —C₄-C₆alkyl;
L₂ is —(CH₂)ₙ— or —C(═O)(CH₂)ₙ—**, where the ** of L₂ indicates the point of attachment to R⁴⁰;
R⁴⁰ is

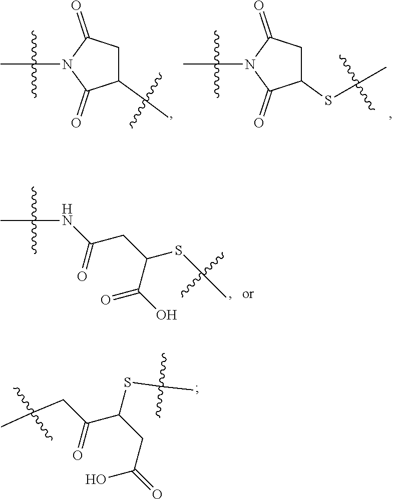 [SMILES: *C1CC(=O)N(*)C1=O ; *SC1CC(=O)N(*)C1=O ; *NC(=O)CC(S*)C(=O)O ; *CC(=O)C(CC(=O)O)S*]

and
each n is independently selected from 1, 2, 3, and 4, and
y is an integer from 1 to 16.

Embodiment 93. The antibody conjugate of Formula (II), Formula (IIa) or Formula (IIb), wherein:
Ab is an anti-DC-SIGN antibody disclosed herein or antigen binding fragment thereof;
R¹ is —NHR²;
R² is —C₄-C₆alkyl;
L₂ is —(CH₂)ₙ—, —((CH₂)ₙO)ₜ(CH₂)ₙ—**, —(CH₂)ₙX₁(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—** or —C(═O)X₂C(═O)(CH₂)ₙNHC(═O)(CH₂)ₙ—**, where the ** of L₂ indicates the point of attachment to R⁴⁰;
R⁴⁰ is

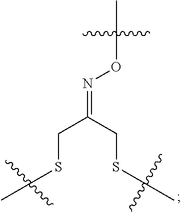 [SMILES: *ON=C(CS*)CS*]

X₁ is

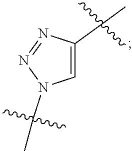 [SMILES: *c1cn(*)nn1]

X₂ is

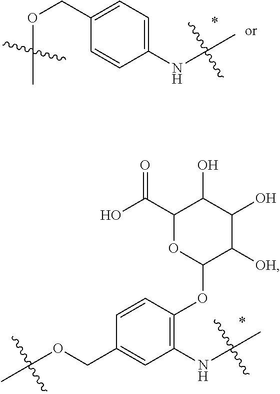 [SMILES: *Nc1ccc(CO*)cc1 ; *Nc1cc(CO*)ccc1OC1OC(C(=O)O)C(O)C(O)C1O]

where the * of X₂ indicates the point of attachment to X₃;
X₃ is

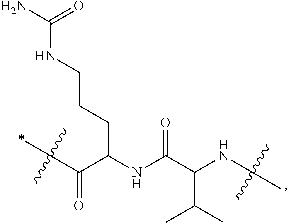

where the * of X₃ indicates the point of attachment to X₂;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Embodiment 94. The antibody conjugate of Formula (II), Formula (IIa) or Formula (IIb), wherein: R¹ is —NHR².

Embodiment 95. The antibody conjugate of Formula (II), Formula (IIa) or Formula (IIb), wherein: R¹ is —NHCHR²R³.

Embodiment 96. The antibody conjugate of Formula (II), Formula (IIa) or Formula (IIb), wherein: R² is —C₄alkyl.

Embodiment 97. The antibody conjugate of Formula (II), Formula (IIa) or Formula (IIb), wherein: R² is —C₅alkyl.

Embodiment 98. The antibody conjugate of Formula (II), Formula (IIa) or Formula (IIb), wherein: R² is —C₆alkyl.

Embodiment 99. The antibody conjugate of Formula (II), Formula (IIa) or Formula (IIb), wherein: R³ is L₁OH;

Embodiment 100. The antibody conjugate of Formula (II), Formula (IIa) or Formula (IIb), wherein: L₁ is —(CH₂)—;

Embodiment 101. The antibody conjugate of Formula (II), Formula (IIa) or Formula (IIb), wherein: L₁ is —(CH₂CH₂)—;

Embodiment 102. The compound of Formula (I), Formula (Ia) or Formula (Ib), wherein: L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O(CH₂)ₙC(═O)NH(CH₂)ₙ—**, —((CH₂)ₙO)ₜ(CH₂)ₙNHC(═O)(CH₂)ₙ—**, where the ** of L₂ indicates the point of attachment to R⁴⁰.

Embodiment 103. The compound of Formula (I), Formula (Ia) or Formula (Ib), wherein: L₂ is —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴⁰.

Embodiment 104. The antibody conjugate of Formula (II), Formula (IIa) or Formula (IIb), wherein: L₂ is —(CH₂)ₙ— or —C(═O)(CH₂)ₙ—**, where the ** of L₂ indicates the point of attachment to R⁴⁰.

Embodiment 105. The antibody conjugate of Formula (II), Formula (IIa) or Formula (IIb), wherein: L₂ is —C(═O)X₂X₃C(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R⁷)₂SS(CH₂)ₙNHC(═O)(CH₂)ₙ—** or —(CH₂)ₙX₂C(═O)(CH₂)ₙNHC(═O)((CH₂)ₙO)ₜ(CH₂)ₙ—**, where the ** of L₂ indicates the point of attachment to R⁴⁰.

Embodiment 106. The antibody conjugate of Formula (II), Formula (IIa) or Formula (IIb), wherein:
R⁴⁰ is

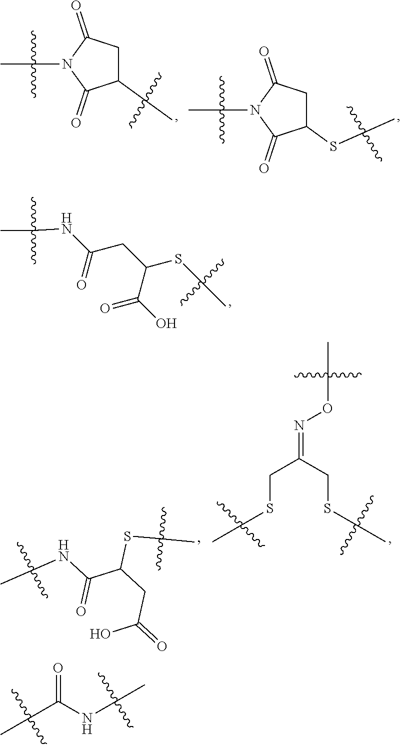 [SMILES: *C1CC(=O)N(*)C1=O ; *SC1CC(=O)N(*)C1=O ; *NC(=O)CC(S*)C(=O)O ; *ON=C(CS*)CS* ; *NC(=O)C(CC(=O)O)S* ; *NC(*)=O]

### or —S—.

Embodiment 107 The antibody conjugate of Formula (II), Formula (IIa) or Formula (IIb), wherein: R⁴⁰ is

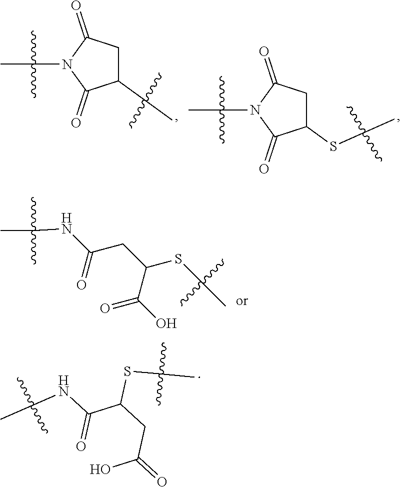 [SMILES: *C1CC(=O)N(*)C1=O ; *SC1CC(=O)N(*)C1=O ; *NC(=O)CC(S*)C(=O)O ; *NC(=O)C(CC(=O)O)S*]

Embodiment 108. The antibody conjugate of Formula (II), Formula (IIa) or Formula (IIb), wherein: R⁴⁰ is

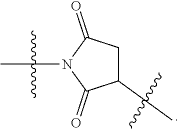 [SMILES: *C1CC(=O)N(*)C1=O]

Embodiment 109. The antibody conjugate of Formula (II), Formula (IIa) or Formula (IIb), wherein: R⁴⁰ is

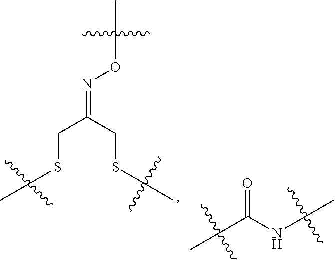 [SMILES: *ON=C(CS*)CS* ; *NC(*)=O]

### —S—.

Embodiment 110. The antibody conjugate of Formula (II), Formula (IIa) or Formula (IIb), wherein: R⁴⁰ is

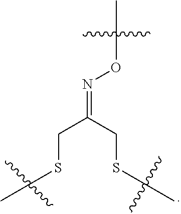 [SMILES: *ON=C(CS*)CS*]

Embodiment 111. The antibody conjugate of Formula (II), Formula (IIa) or Formula (IIb), wherein: R⁴⁰ is

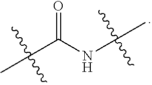 [SMILES: *NC(*)=O]

Embodiment 112. The antibody conjugate of Formula (II), Formula (IIa) or Formula (IIb), wherein:
R⁴⁰ is

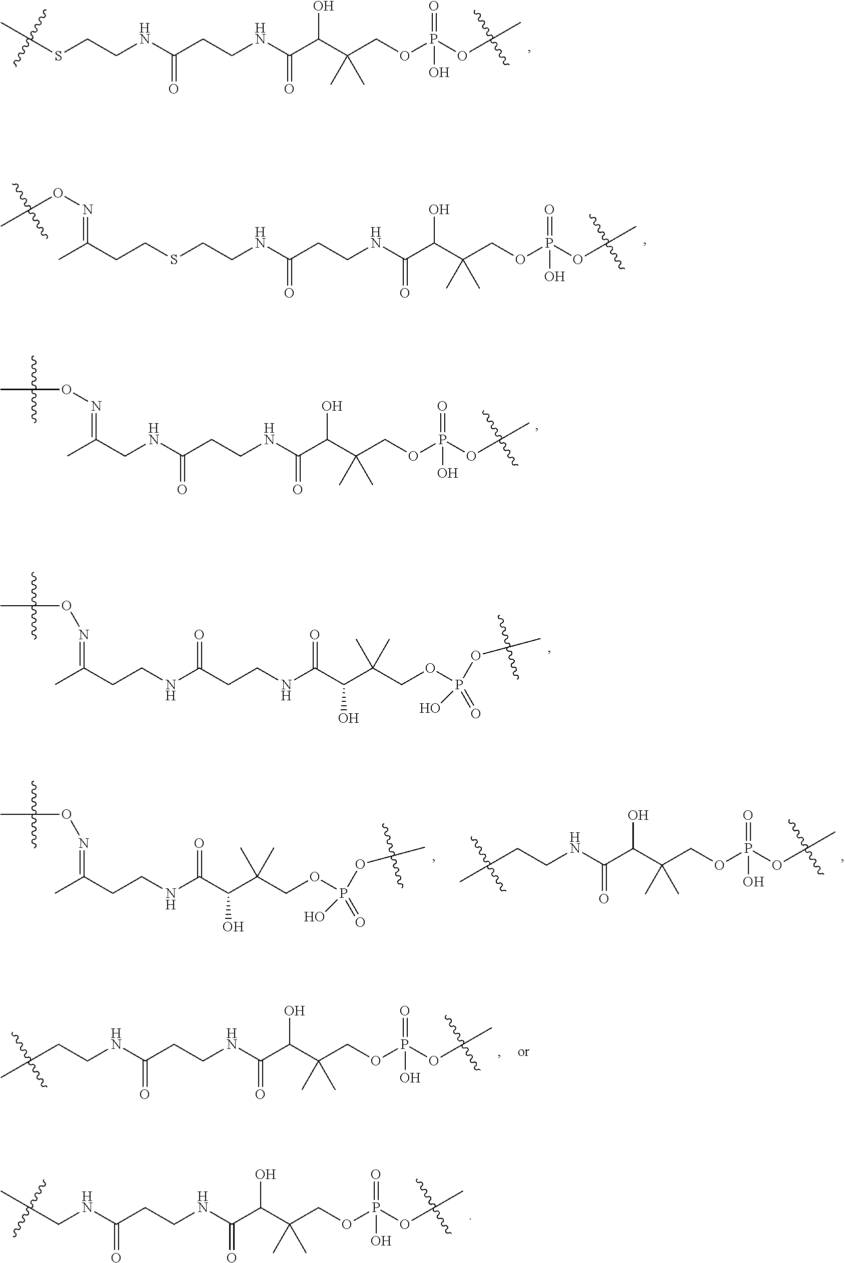

Embodiment 113. The antibody conjugate of Formula (II), Formula (IIa) or Formula (IIb), wherein: X₁ is

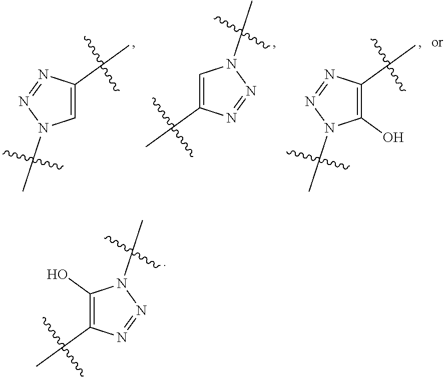 [SMILES: *c1cn(*)nn1 ; *c1cn(*)nn1 ; *c1nnn(*)c1O ; *c1nnn(*)c1O]

Embodiment 114. The antibody conjugate of Formula (II), Formula (IIa) or Formula (IIb), wherein: X₁ is

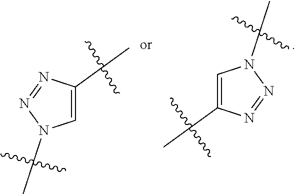 [SMILES: *c1cn(*)nn1 ; *c1cn(*)nn1]

Embodiment 115. The antibody conjugate of Formula (II), Formula (IIa) or Formula (IIb), wherein: X₁ is

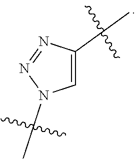 [SMILES: *c1cn(*)nn1]

Embodiment 116. The antibody conjugate of Formula (II), Formula (IIa) or Formula (IIb), wherein: X₂ is

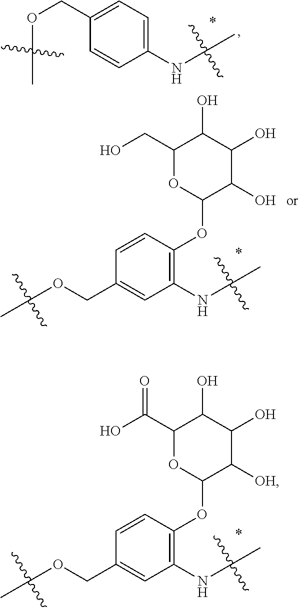

where the * of X₂ indicates the point of attachment to X₃.

Embodiment 117. The antibody conjugate of Formula (II), Formula (IIa) or Formula (IIb), wherein: X₂ is

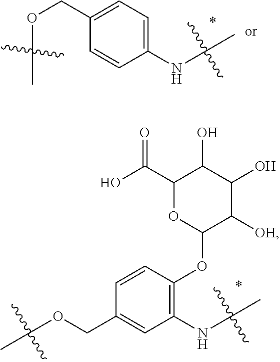 [SMILES: *Nc1ccc(CO*)cc1 ; *Nc1cc(CO*)ccc1OC1OC(C(=O)O)C(O)C(O)C1O]

where the * of X₂ indicates the point of attachment to X₃.

Embodiment 118. The antibody conjugate of Formula (II), Formula (IIa) or Formula (IIb), wherein: X₂ is

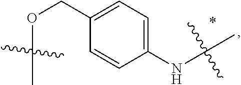 [SMILES: *Nc1ccc(CO*)cc1]

where the * of X₂ indicates the point of attachment to X₃.

Embodiment 119. The antibody conjugate of Formula (II), Formula (IIa) or Formula (IIb), wherein: X₂ is

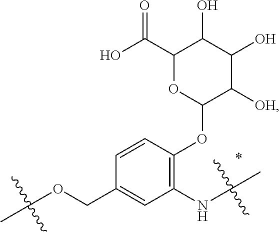 [SMILES: *Nc1cc(CO*)ccc1OC1OC(C(=O)O)C(O)C(O)C1O]

where the * of X₂ indicates the point of attachment to X₃.

Embodiment 120. The antibody conjugate of Formula (II), Formula (IIa) or Formula (IIb), wherein: X₂ is

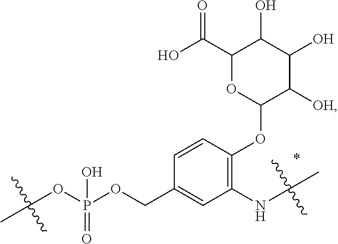 [SMILES: *Nc1cc(COP(=O)(O)O*)ccc1OC1OC(C(=O)O)C(O)C(O)C1O]

where the * of X₂ indicates the point of attachment to X₃.

Embodiment 121. The antibody conjugate of Formula (II), Formula (IIa) or Formula (IIb), wherein: X₃ is

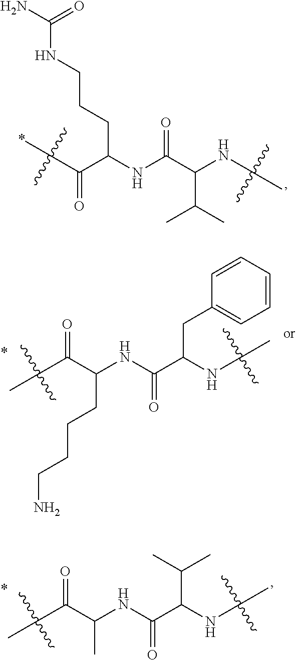

where the * of X₃ indicates the point of attachment to X₂.

Embodiment 122. The antibody conjugate of Formula (II), Formula (IIa) or Formula (IIb), wherein: X₃ is

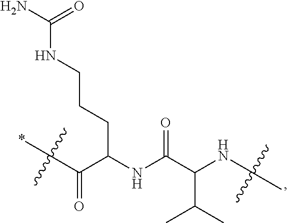

where the * of X₃ indicates the point of attachment to X₂.

Embodiment 123. The antibody conjugate of Formula (II), Formula (IIa) or Formula (IIb), wherein: X₃ is

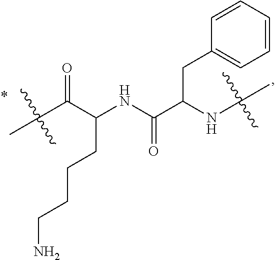 [SMILES: *NC(Cc1ccccc1)C(=O)NC(CCCCN)C(*)=O]

where the * of X₃ indicates the point of attachment to X₂.

Embodiment 124. The antibody conjugate of Formula (II), Formula (IIa) or Formula (IIb), wherein: X₃ is

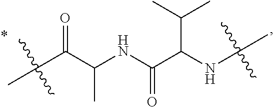 [SMILES: *NC(C(=O)NC(C)C(*)=O)C(C)C]

where the * of X₃ indicates the point of attachment to X₂.

Embodiment 125. The antibody conjugate of Formula (II), Formula (IIa) or Formula (IIb), wherein: each m is independently selected from 1, 2, 3, and 4.

Embodiment 126. The antibody conjugate of Formula (II), Formula (IIa) or Formula (IIb), wherein: each m is 1 or 2.

Embodiment 127. The antibody conjugate of Formula (II), Formula (IIa) or Formula (IIb), wherein: each n is independently selected from 1, 2, 3, and 4.

Embodiment 128. The antibody conjugate of Formula (II), Formula (IIa) or Formula (IIb), wherein: each n is 2 or 3.

Embodiment 129. The antibody conjugate of Formula (II), Formula (IIa) or Formula (IIb), wherein: each t is independently selected from 1, 2, 3, 4, 5, 6, 7, 8, 9, 10, 11, 12, 13, 14, 15, 16, 17 and 18.

Embodiment 130. The antibody conjugate of Formula (II), Formula (IIa) or Formula (IIb), wherein: each t is independently selected from 1, 2, 3, 4, 5 and 6.

Embodiment 131. The antibody conjugate of Formula (II), Formula (IIa) or Formula (IIb), wherein y is an integer from 1 to 16.

Embodiment 132. The antibody conjugate of Formula (II), Formula (IIa) or Formula (IIb), wherein y is an integer from 1 to 8.

Embodiment 133. The antibody conjugate of Formula (II), Formula (IIa) or Formula (IIb), wherein y is an integer from 1 to 4.

Embodiment 134. The antibody conjugate of Formula (II), Formula (IIa) or Formula (IIb) selected from:

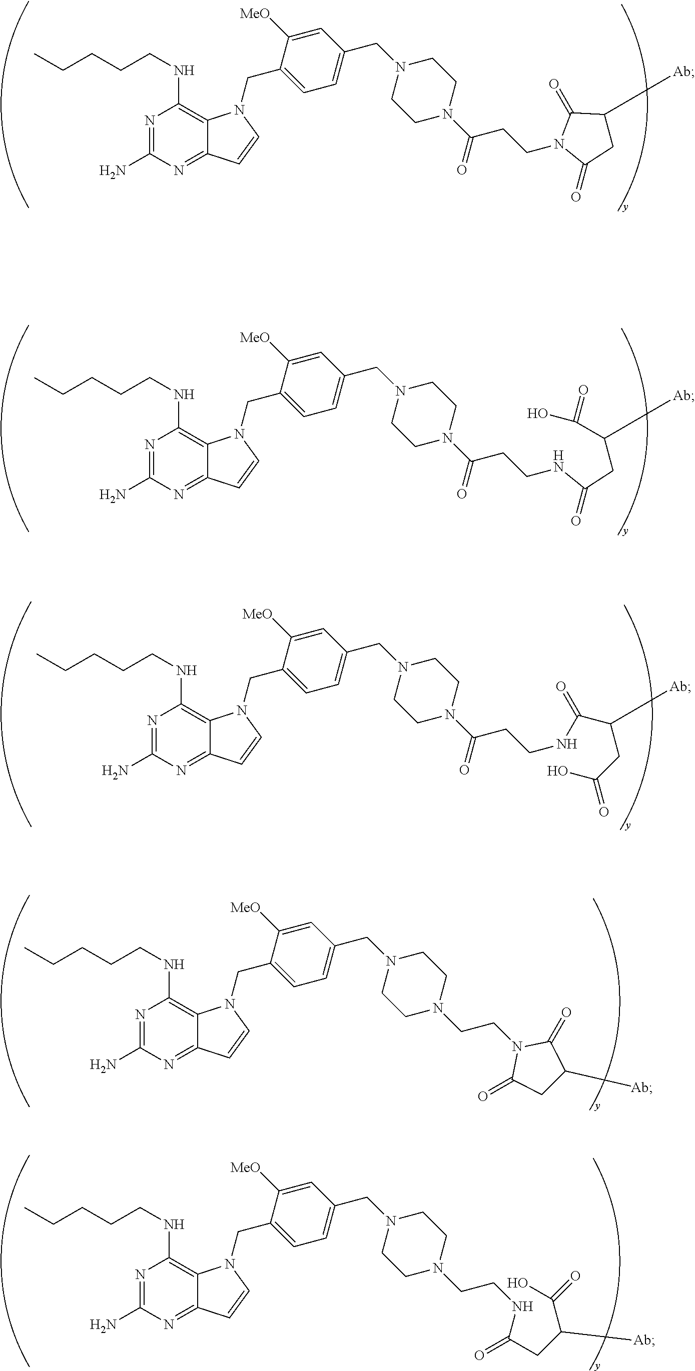

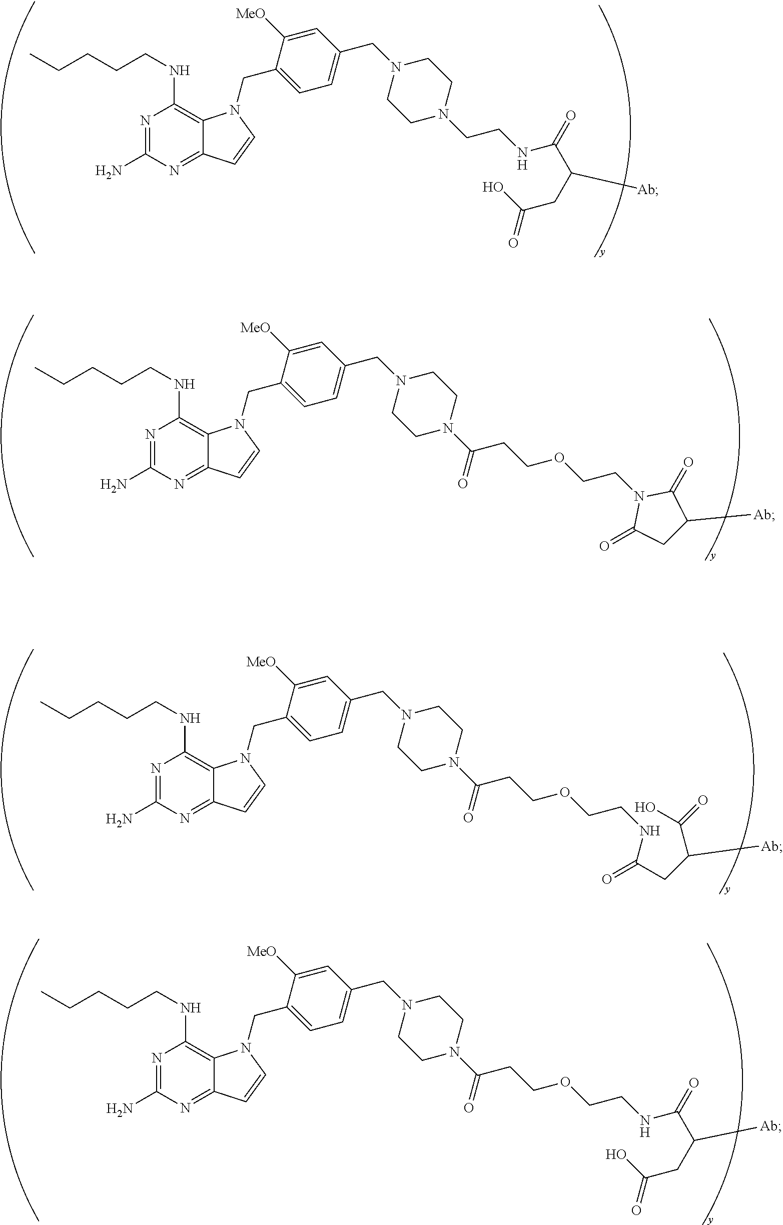

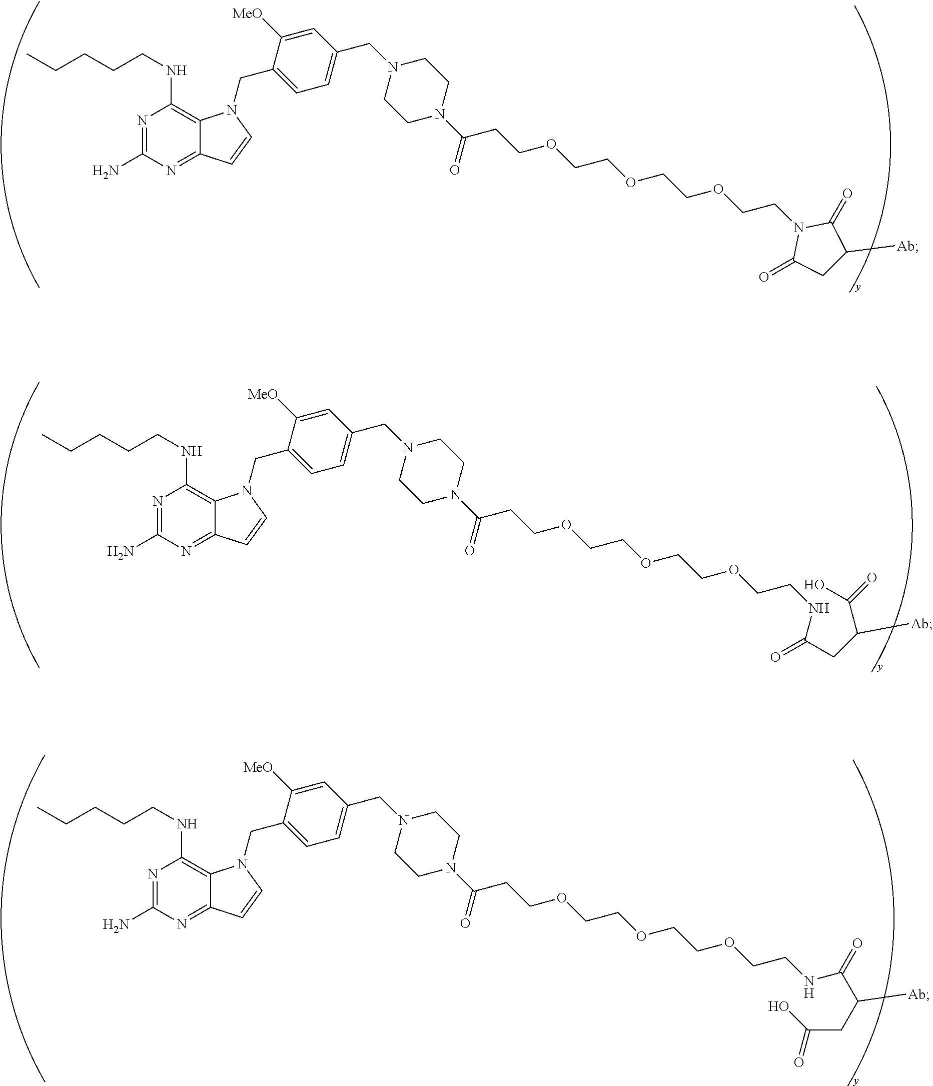

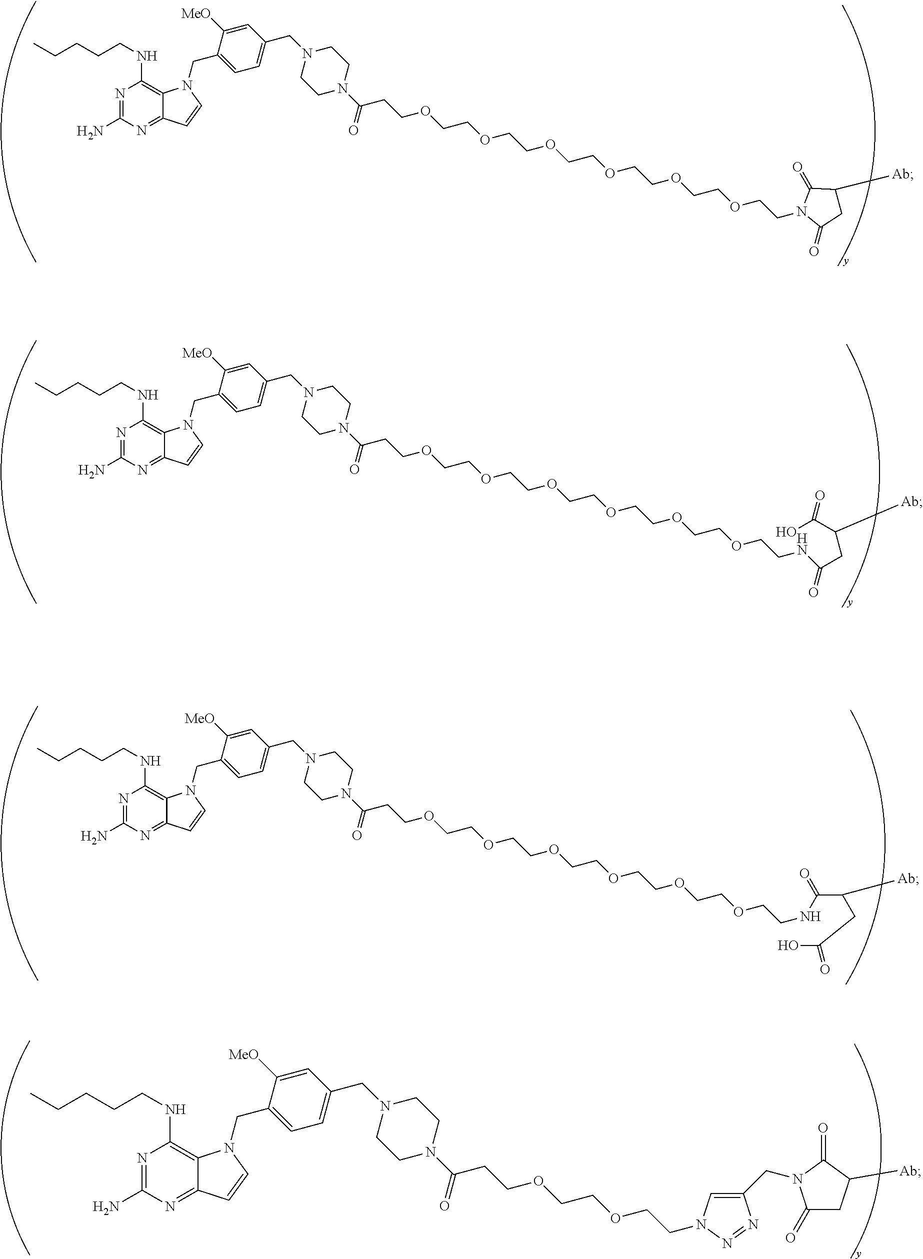

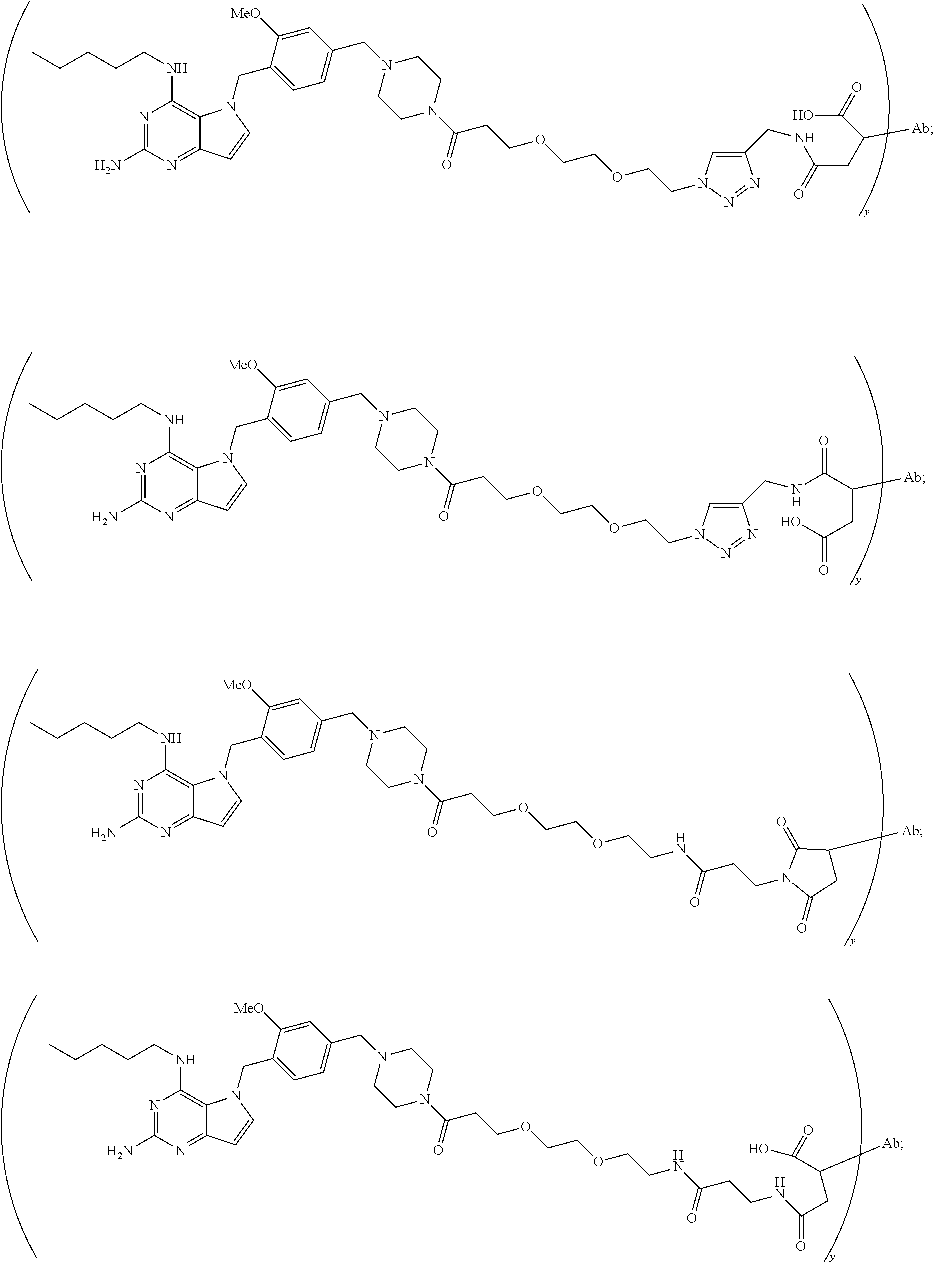

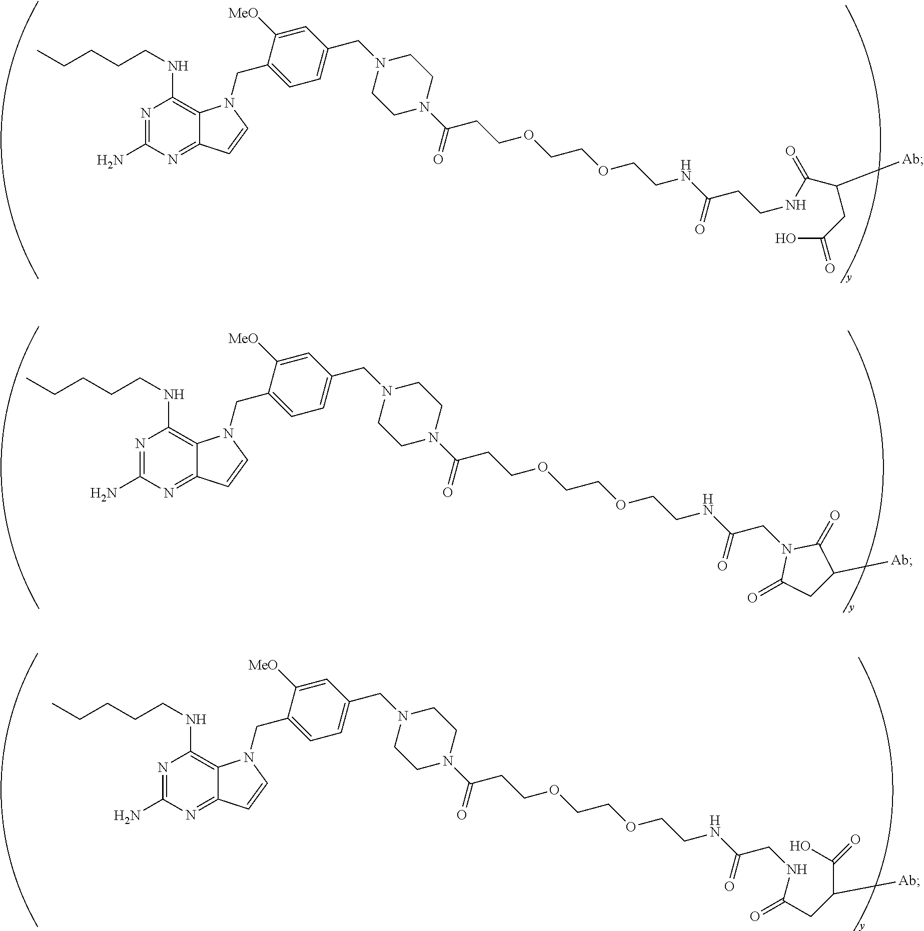

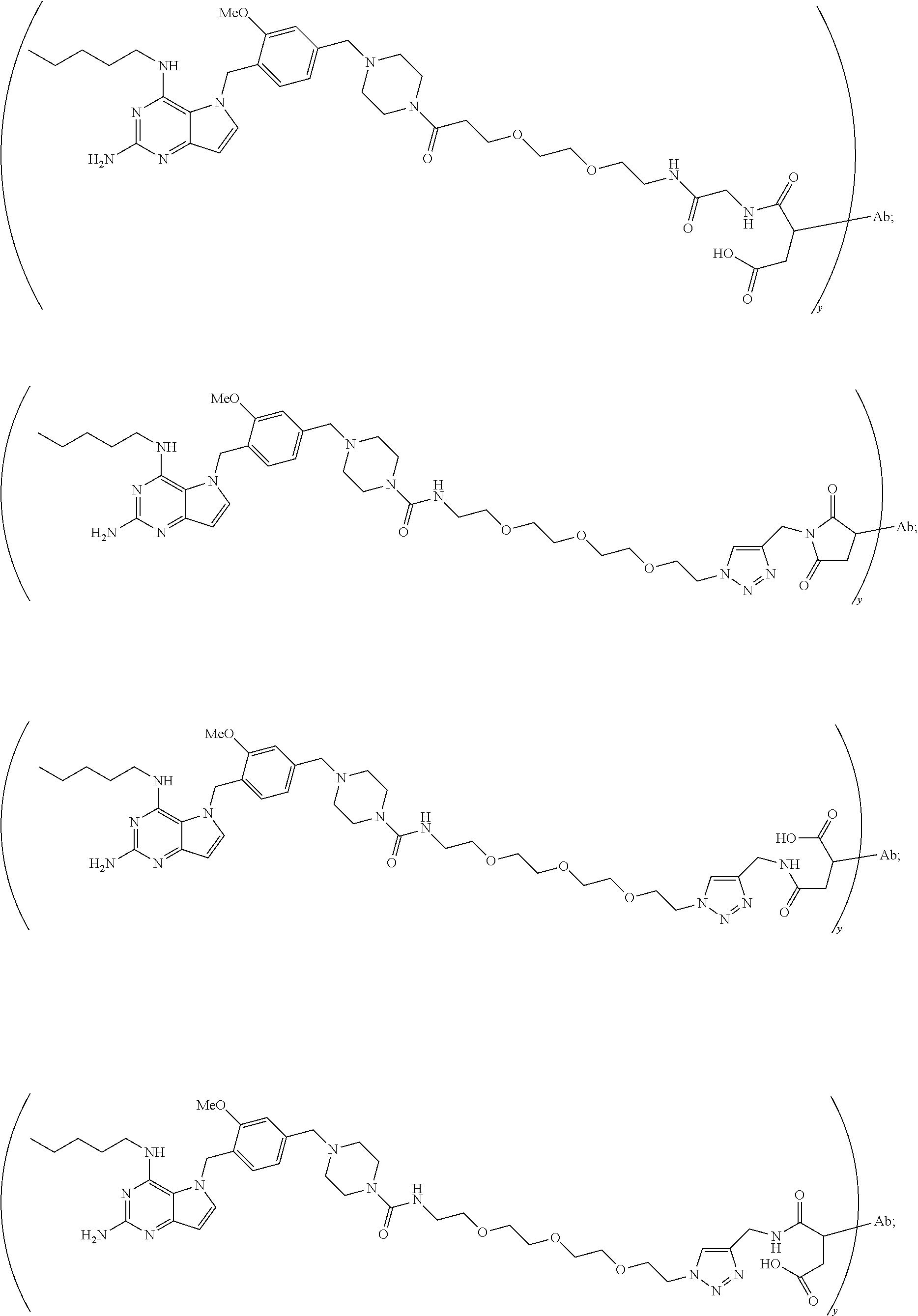

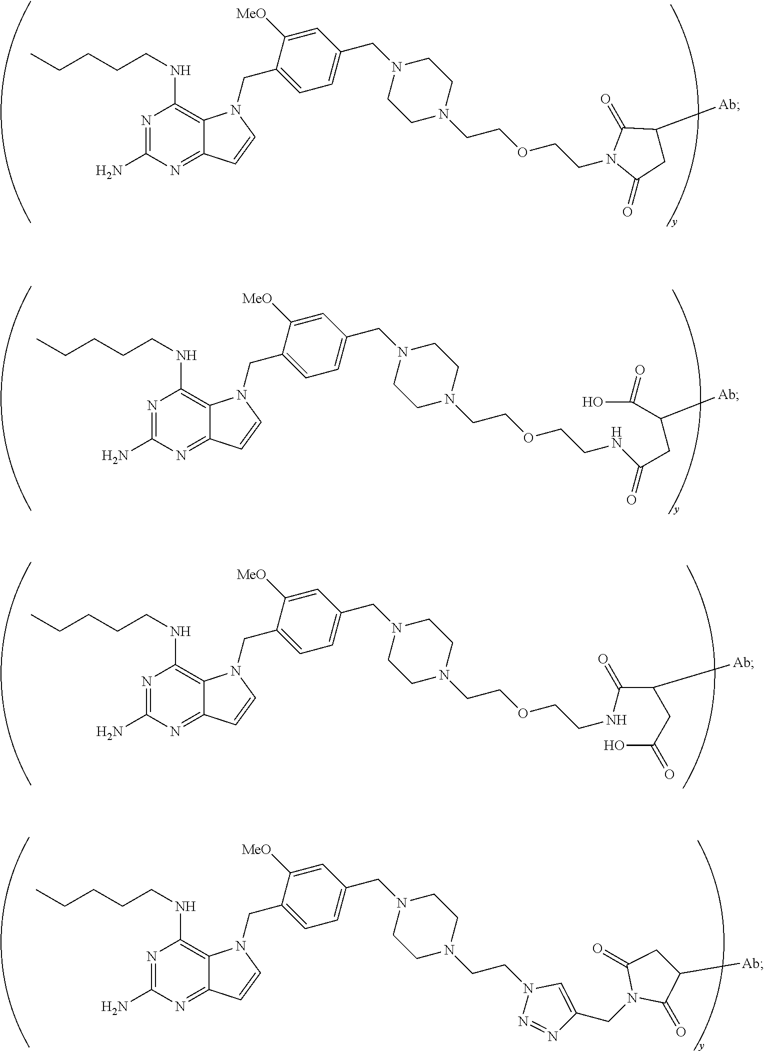

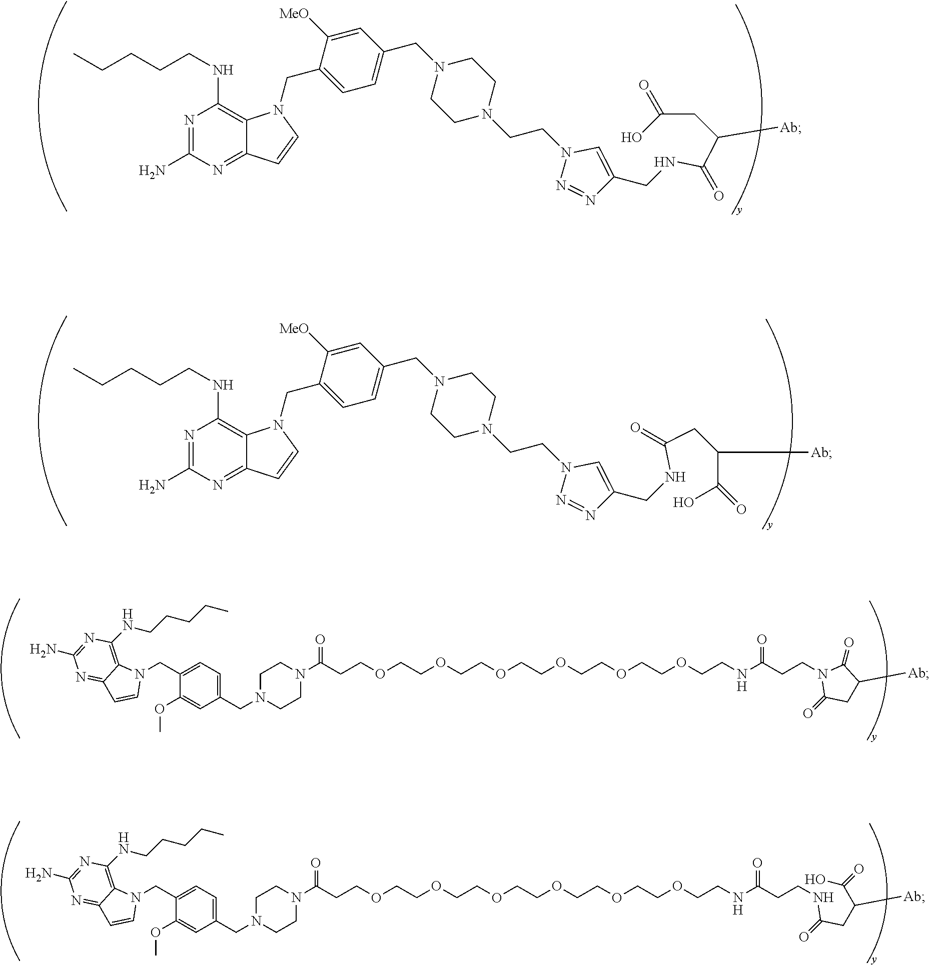

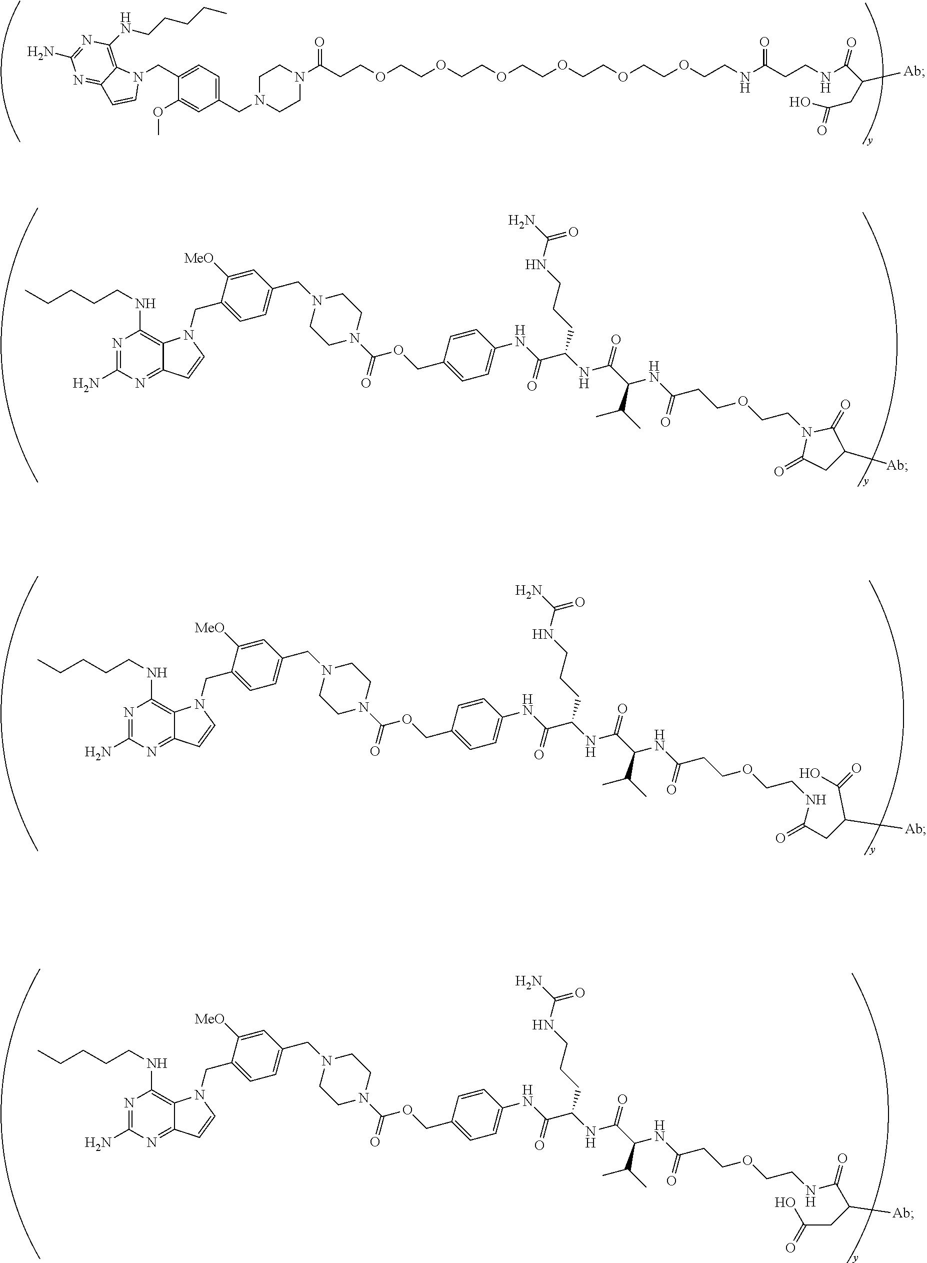

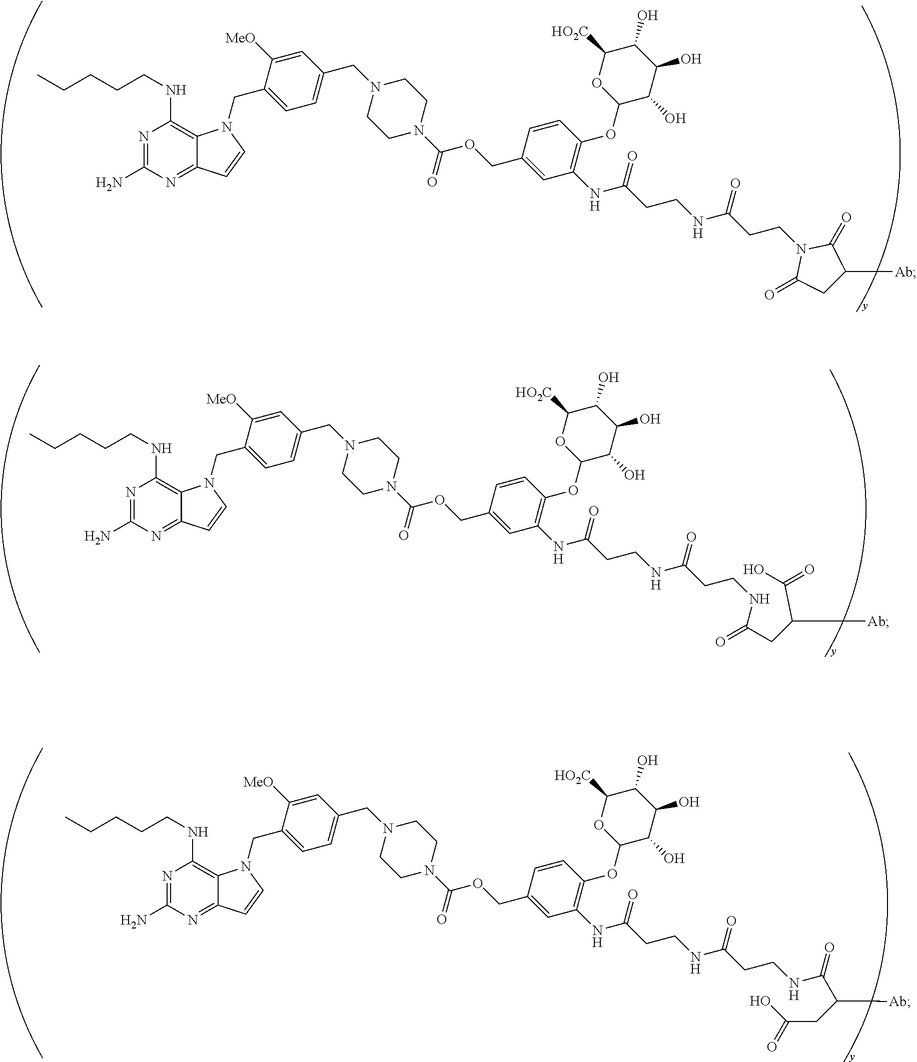

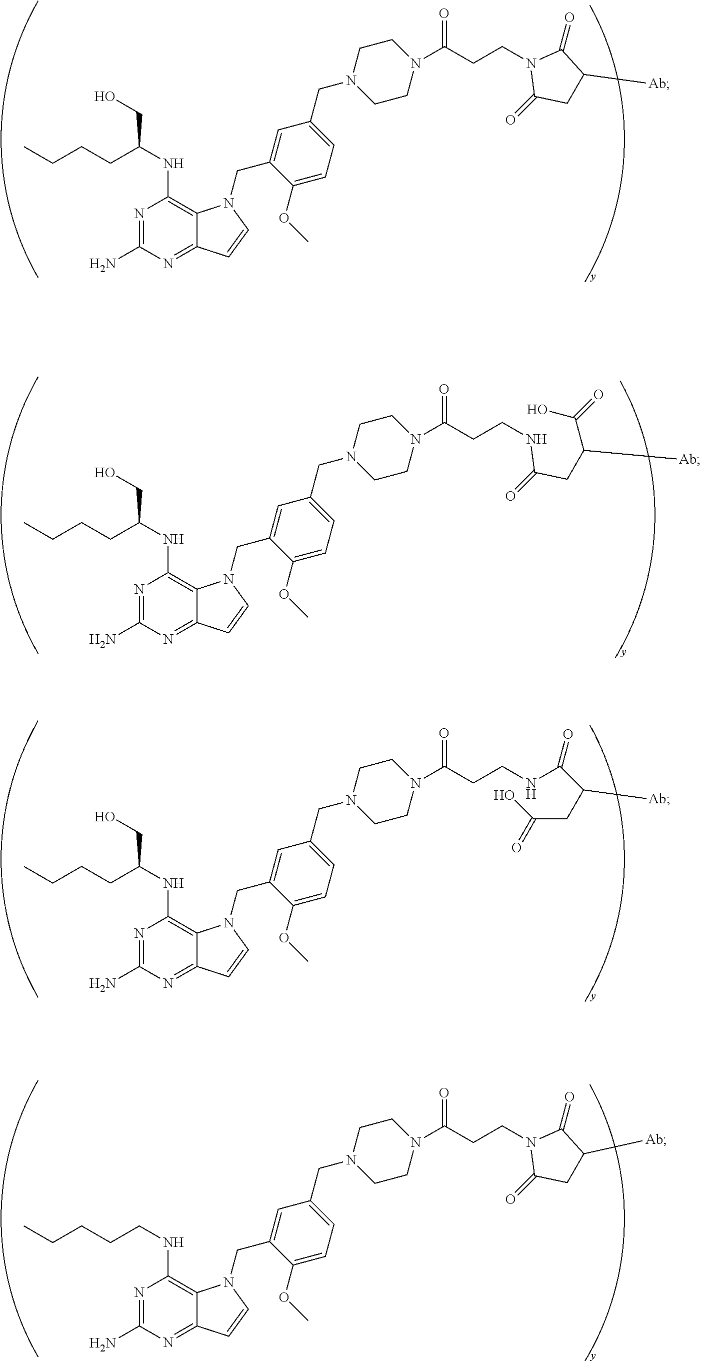

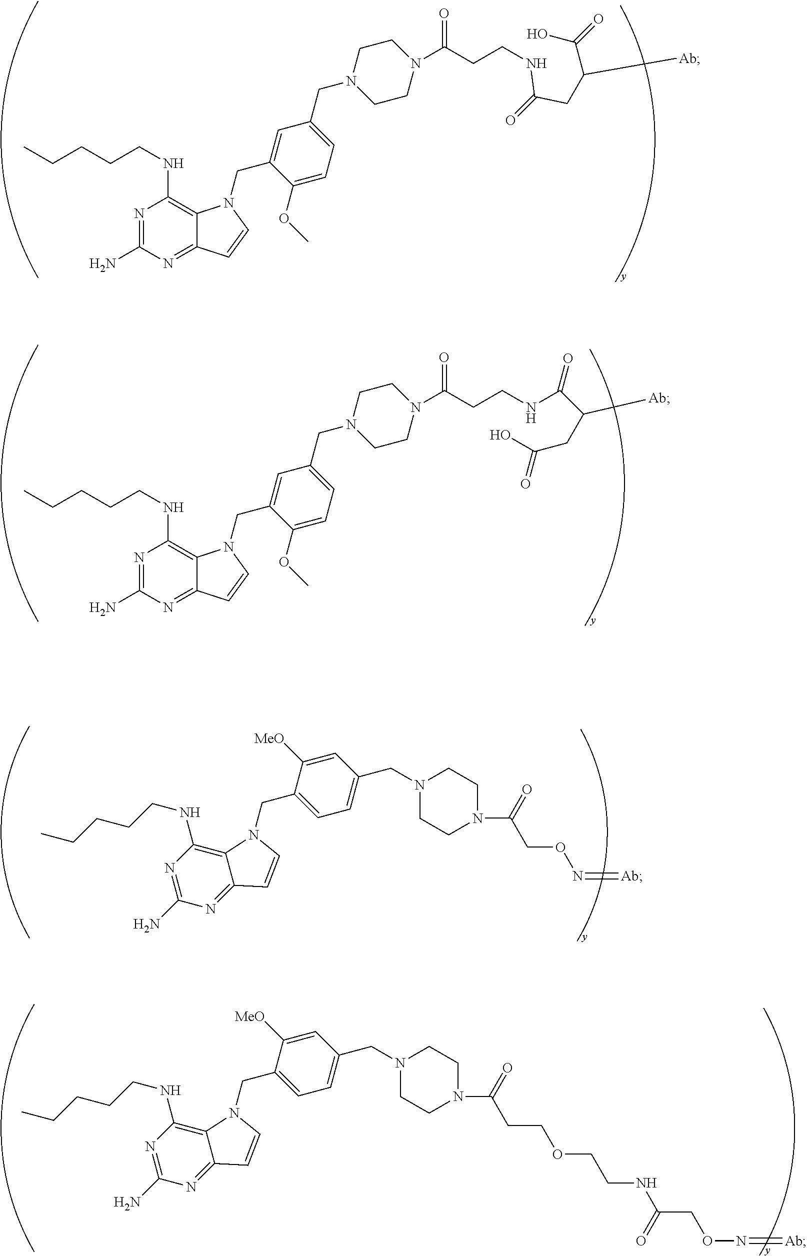

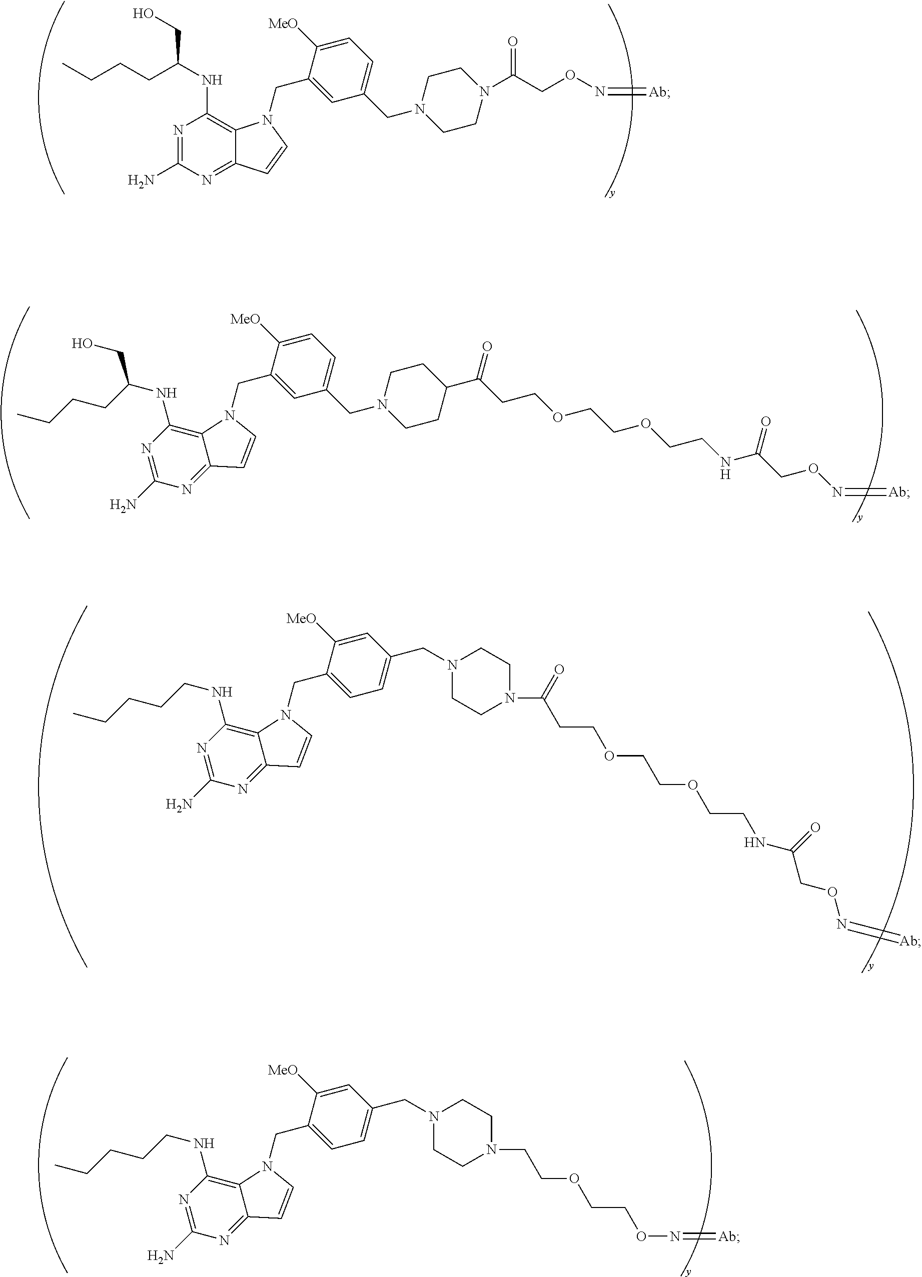

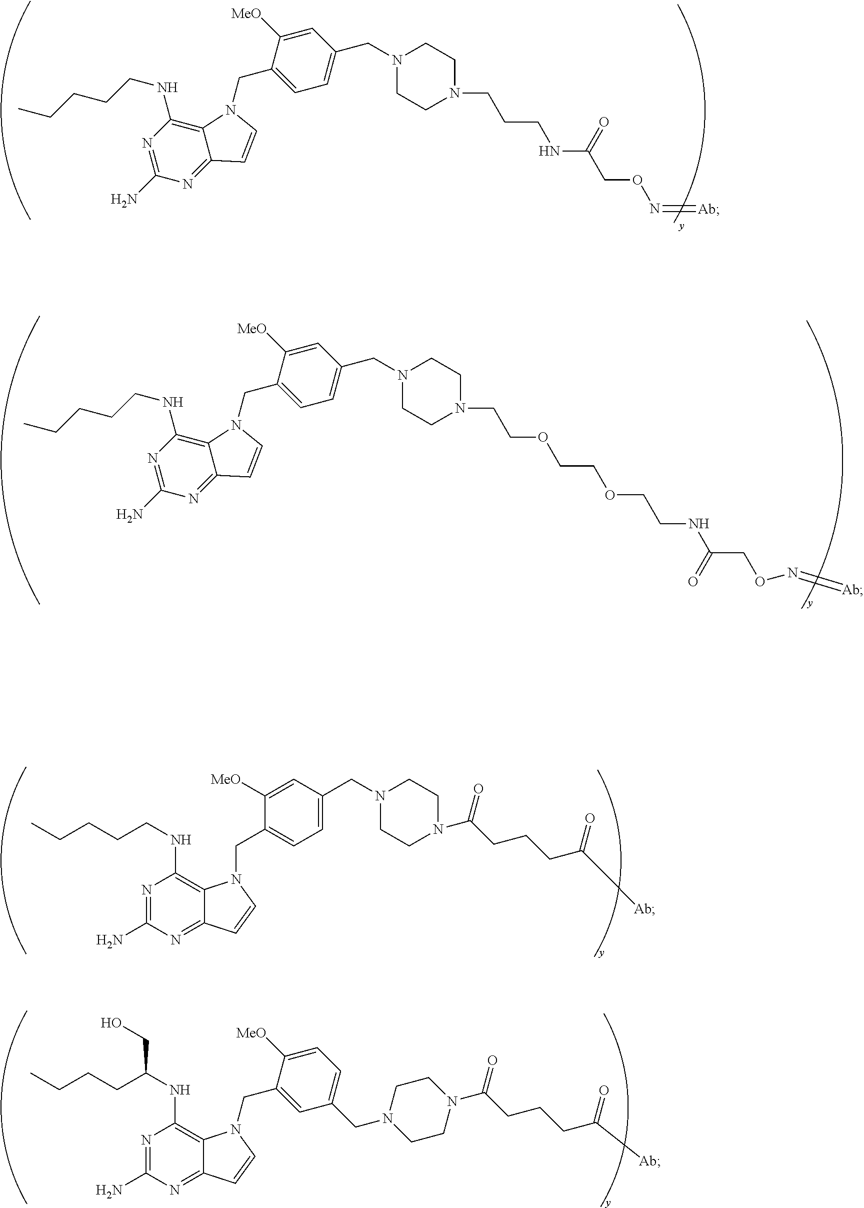

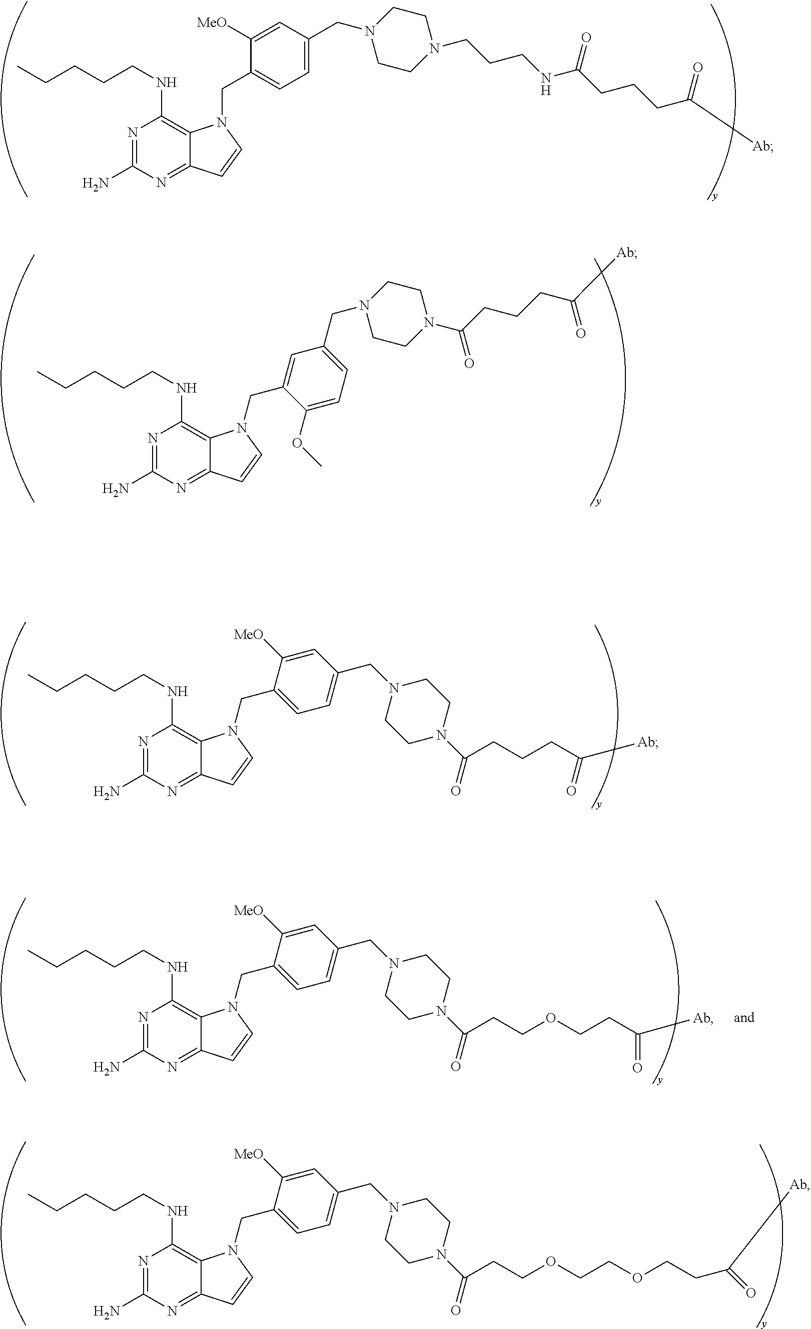

wherein y is an integer from 1 to 4 and Ab is an anti-DC-SIGN antibody disclosed herein or antigen binding fragment thereof.

Embodiment 135. The antibody conjugate of Formula (II), Formula (IIa) or Formula (IIb) selected from:

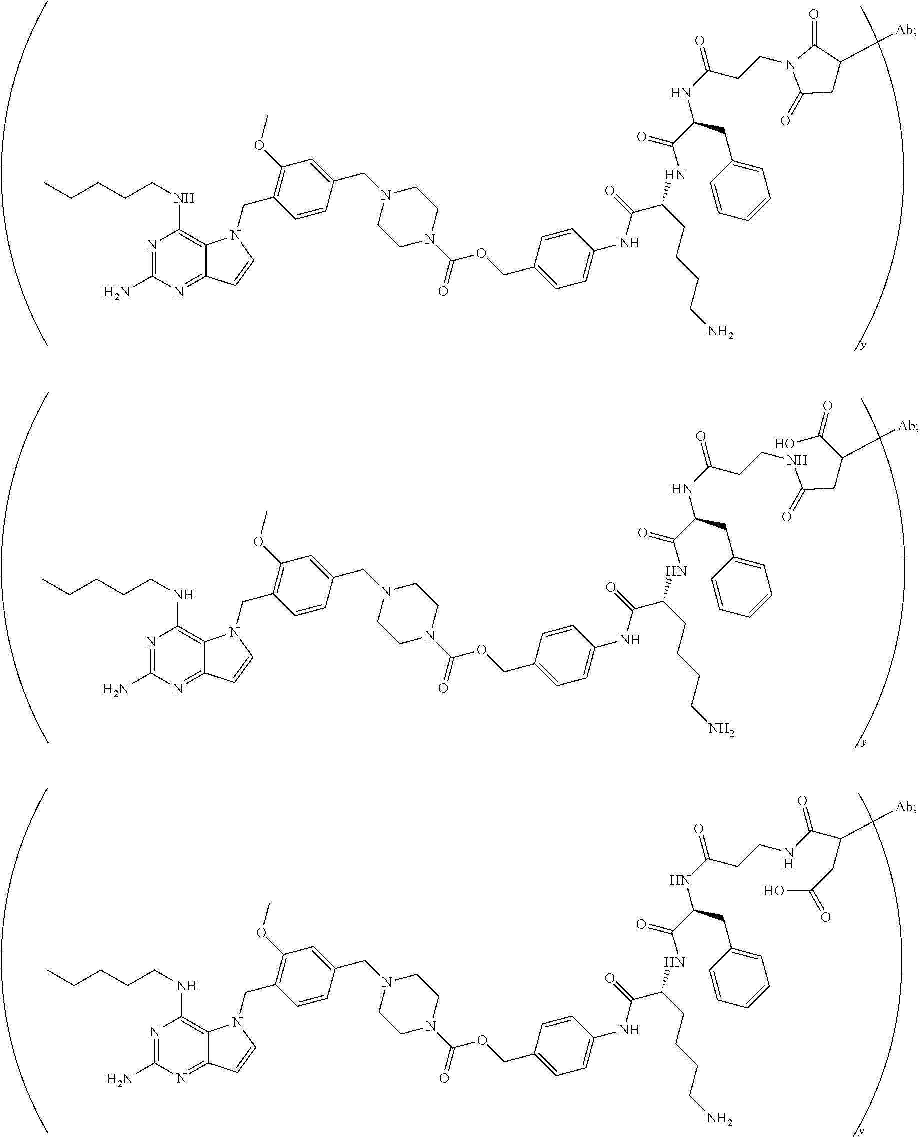

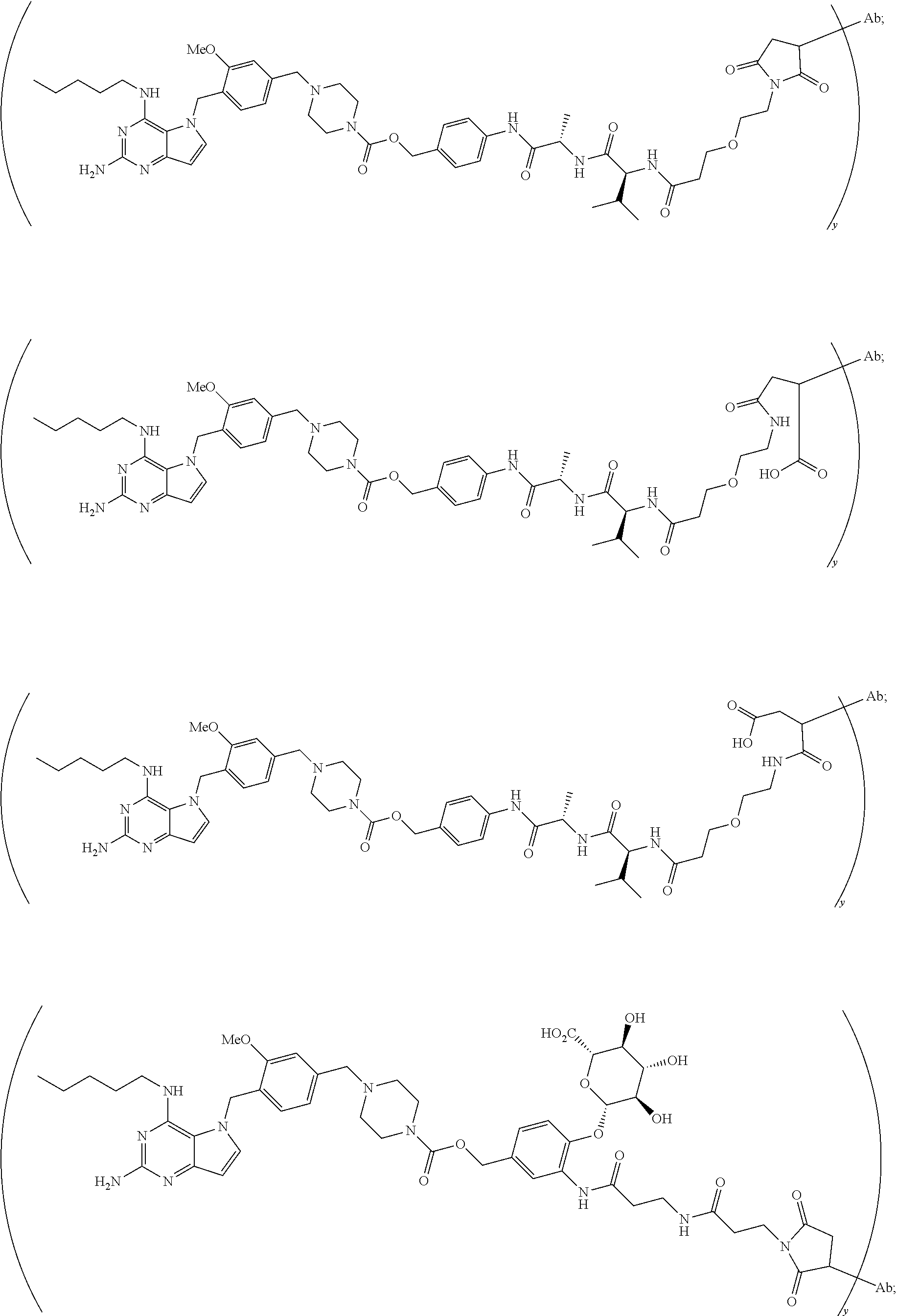

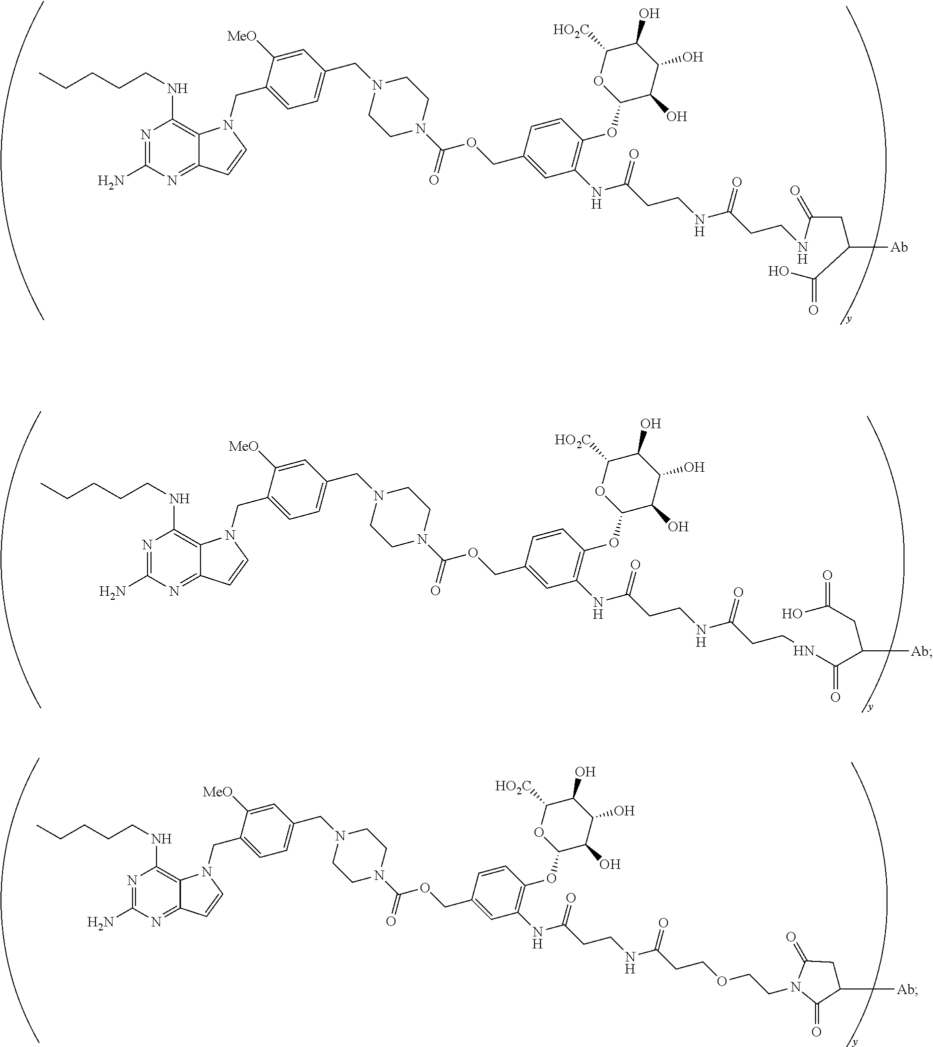

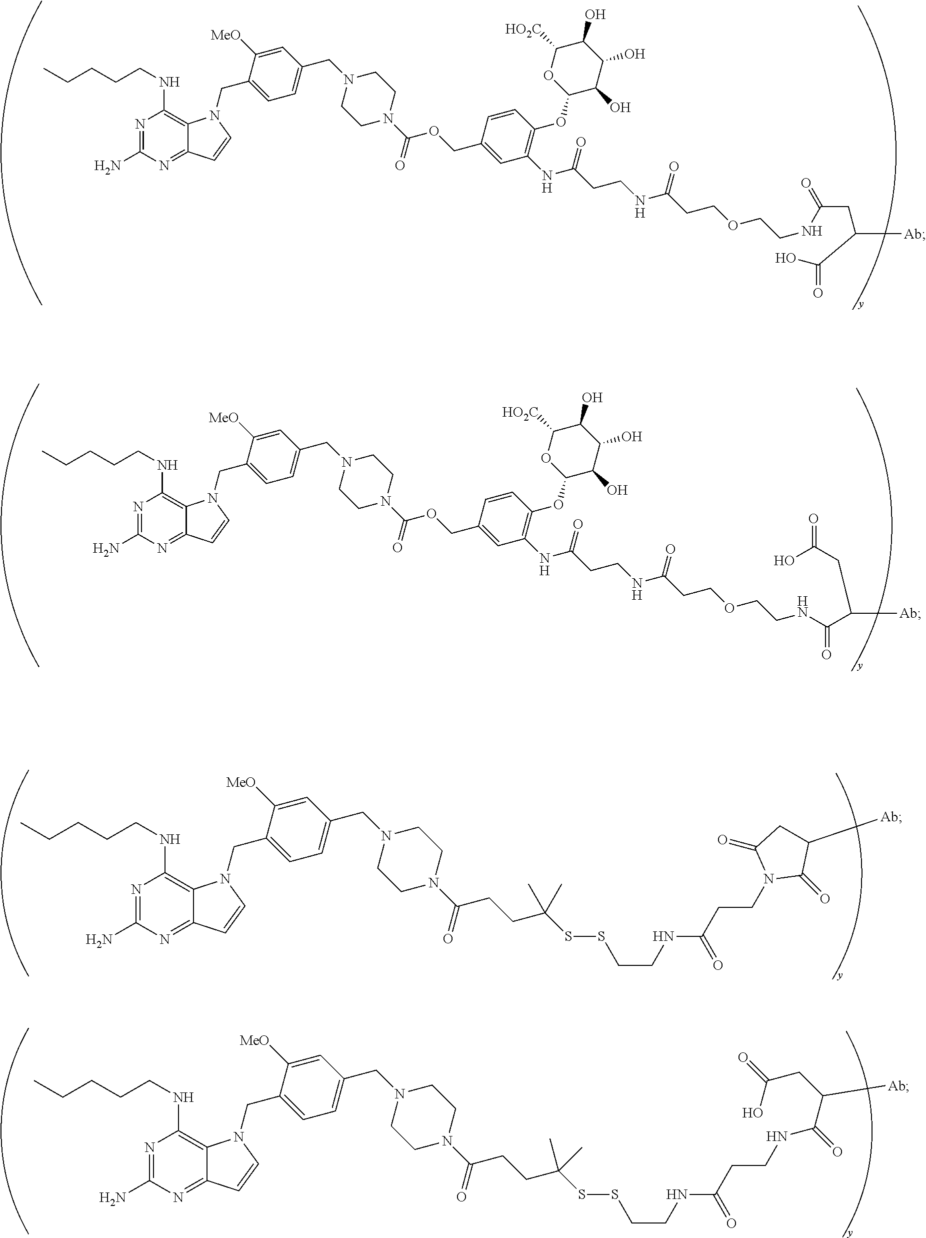

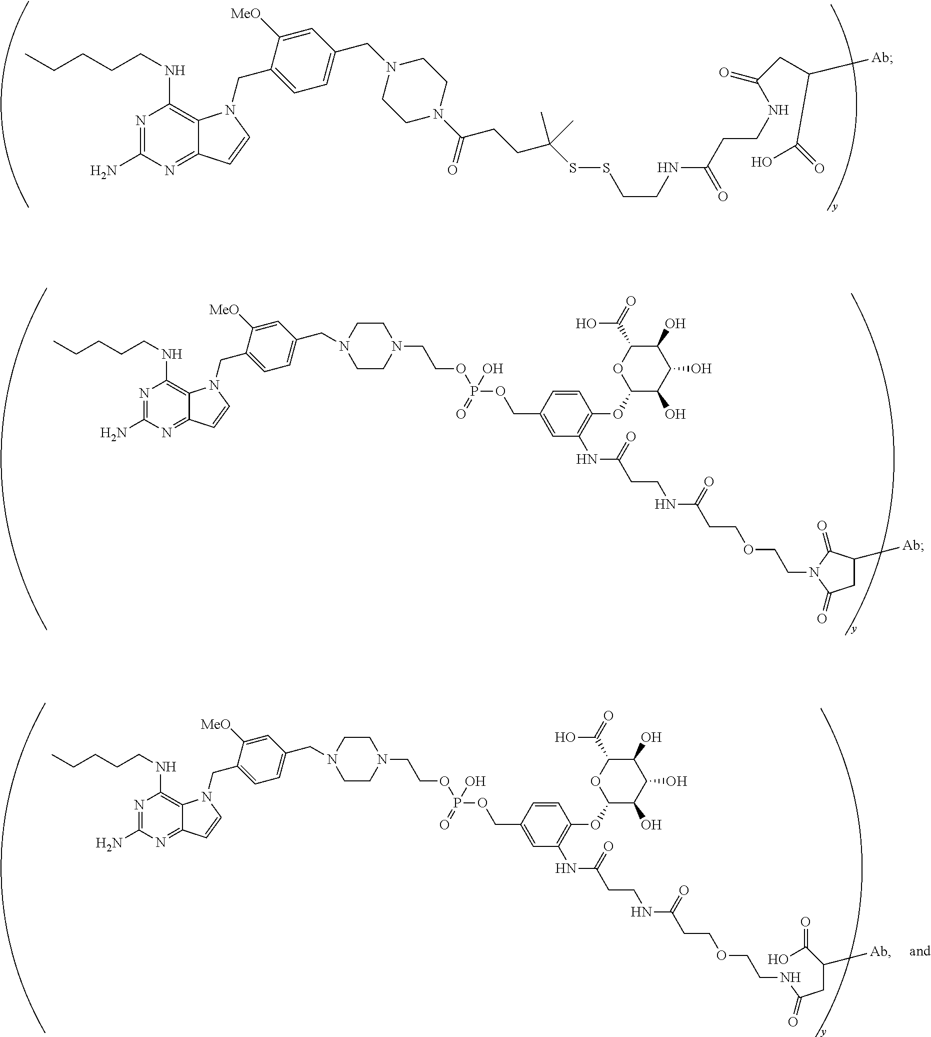

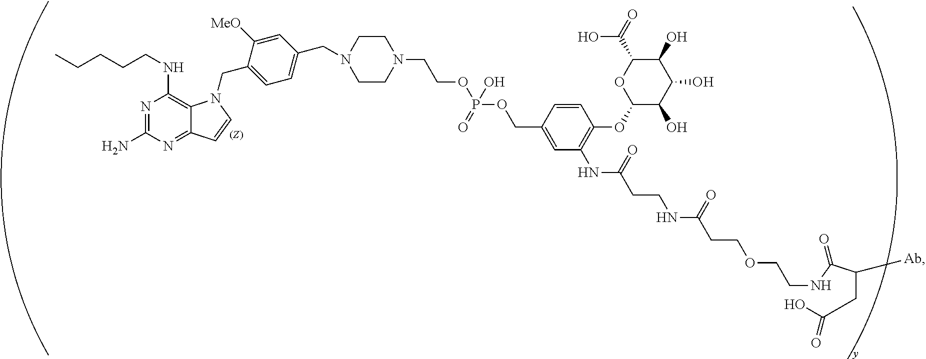

wherein y is an integer from 1 to 4 and Ab is an anti-DC-SIGN antibody disclosed herein or antigen binding fragment thereof.

In some embodiments, the antibody conjugates of the invention comprise a RIG-1 agonist and have the structure of Formula (V):

(RIGIa-L-R⁴⁰)_{y}-Ab Formula (V)

wherein:
RIGIa is a RIG-1 agonist selected from:

a)
(SEQ ID NO: 338)
5′ ppp-GGACGUACGC (UX_{M}CG) GCGUACGUCC-3′
or
b)
(SEQ ID NO: 339)
5′ OH-GGACGUACGC (UX_{M}CG) GCGUACGUCC-3′

where:
   ppp-G is

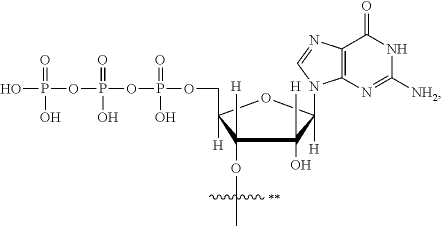

where the ** of ppp-G is the point of attachment toward the 3′ end;
   OH-G is

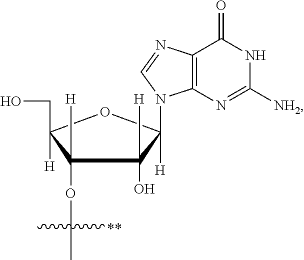

where the ** of OH-G is the point of attachment toward the 3′ end;
   G is

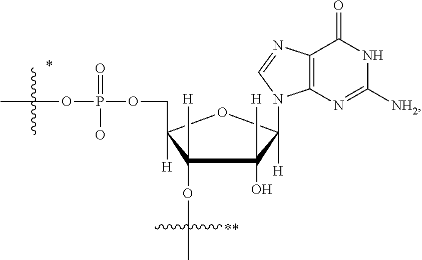

where the * of G is the point of attachment toward the 5′ end and the ** of G is the point of attachment toward the 3′ end;
   A is

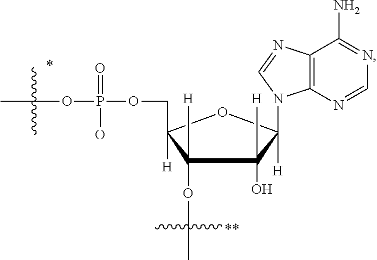

where the * of A is the point of attachment toward the 5′ end and the ** of A is the point of attachment the point of attachment toward the 3′ end;
   C is

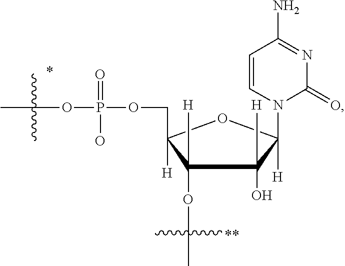

where the * of C is the point of attachment toward the 5′ end and the ** of C is the point of attachment toward the 3′ end;
   or if C is in a 3′ terminal position, then C is

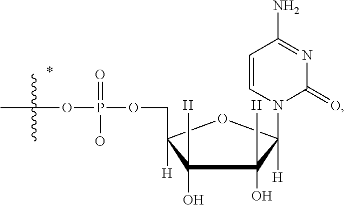

where the * of C is the point of attachment toward the 5′ end;
   U is

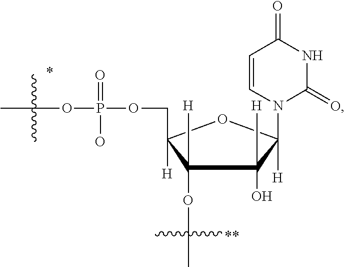

where the * of U is the point of attachment toward the 5′ end and the ** of U is the point of attachment toward the 3′ end;
   X_{M} is

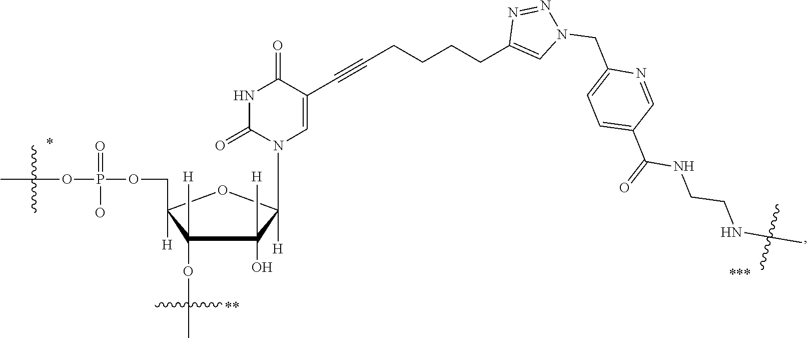

where the * of X_{M} is the point of attachment toward the 5′ end, the ** of X_{M} is the point of attachment toward the 3′ end and the *** of X_{M} is the point of attachment to L;
Ab is an antibody or antigen binding fragment thereof that specifically binds to human DC-SIGN;
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—**; —C(═O)X₂X₃((CH₂)ₙO)ₜ(CH₂)ₙ—**, —C(═O)X₂X₃C(═O)(CH₂)ₘO(CH₂)ₘC(═O)—**; —(CH₂)ₙNHC(═O)(CH₂)ₙ—, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)O(CH₂)ₙC(R₇)₂SS(CH₂)ₙNHC(═O)(CH₂)ₙ—** or —C(═O)(CH₂)ₙC(═O)NH(CH₂)ₙ—**, where the ** of L indicates the point of attachment to R⁴⁰;
R⁴⁰ is

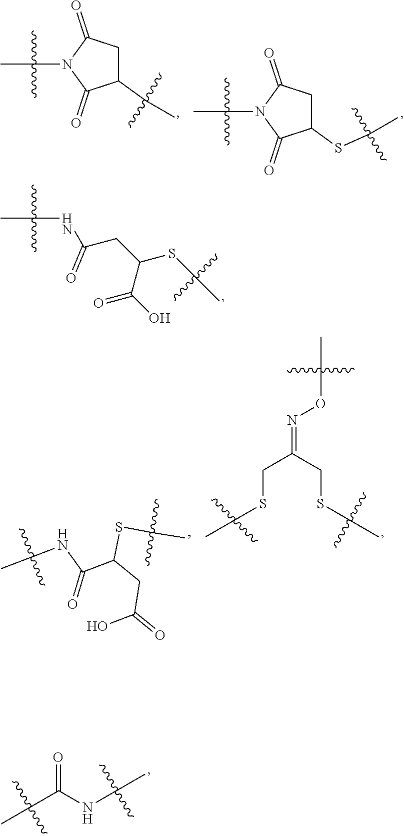 [SMILES: *C1CC(=O)N(*)C1=O ; *SC1CC(=O)N(*)C1=O ; *NC(=O)CC(S*)C(=O)O ; *ON=C(CS*)CS* ; *NC(=O)C(CC(=O)O)S* ; *NC(*)=O]

—S—, —NHC(═O)CH₂—, —S(═O)₂CH₂CH₂—, —(CH₂)₂S(═O)₂CH₂CH₂—, —NHS(═O)₂CH₂CH₂, —NHC(═OCH)CH₂—, —CH₂NHCH₂CH₂—, —NHCH₂CH₂—,

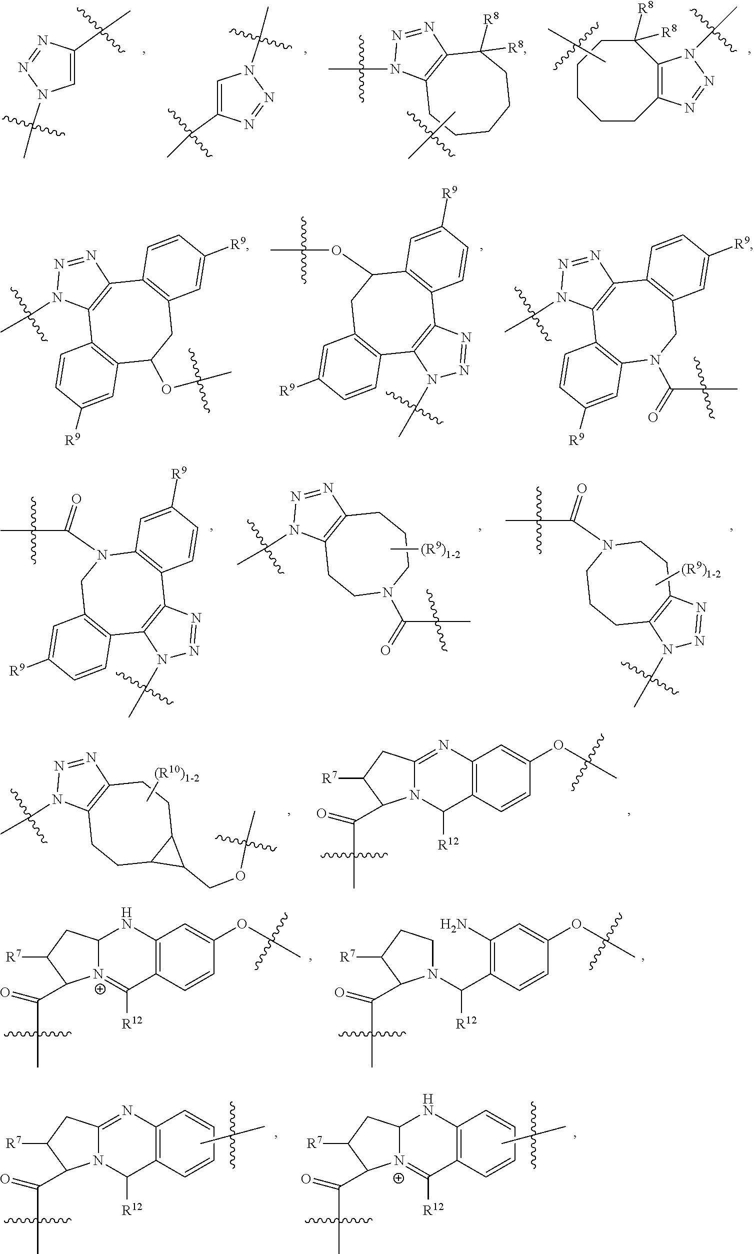

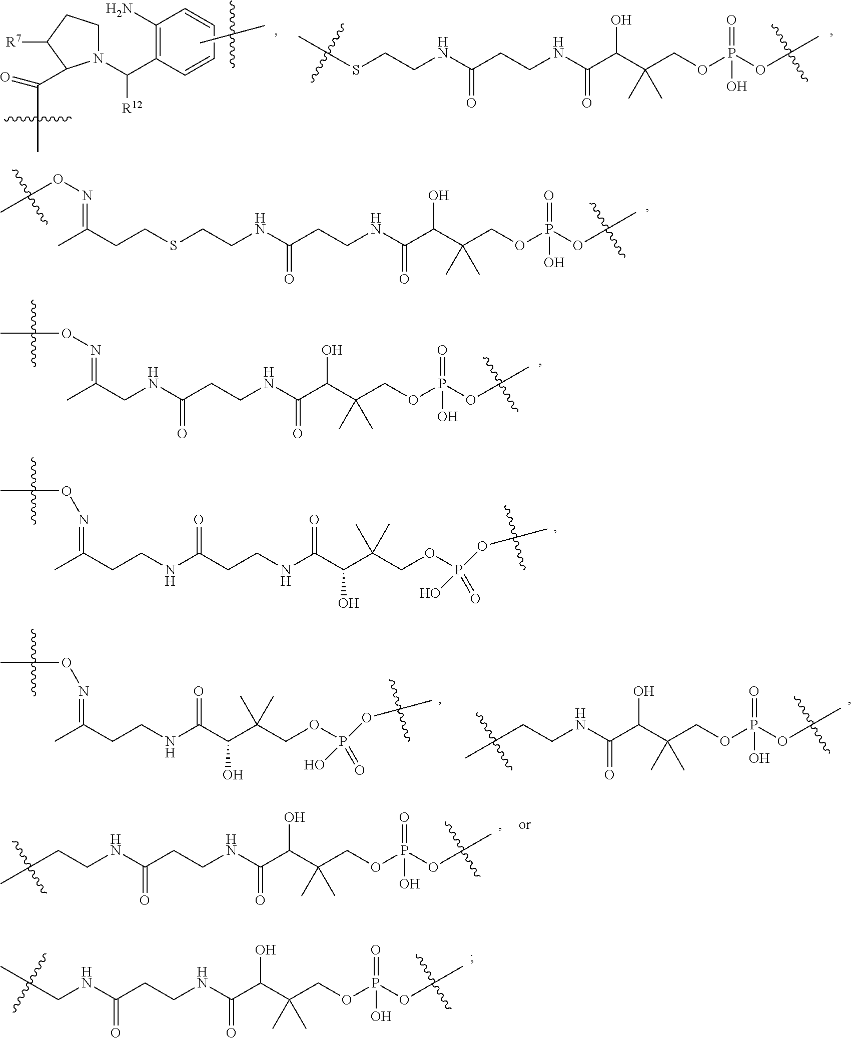

X₁ is

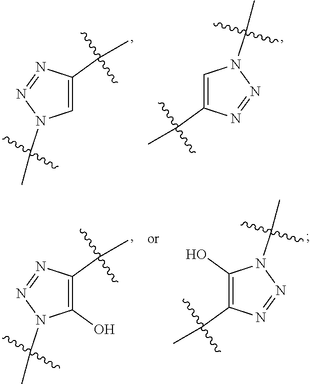 [SMILES: *c1cn(*)nn1 ; *c1cn(*)nn1 ; *c1nnn(*)c1O ; *c1nnn(*)c1O]

X₂ is

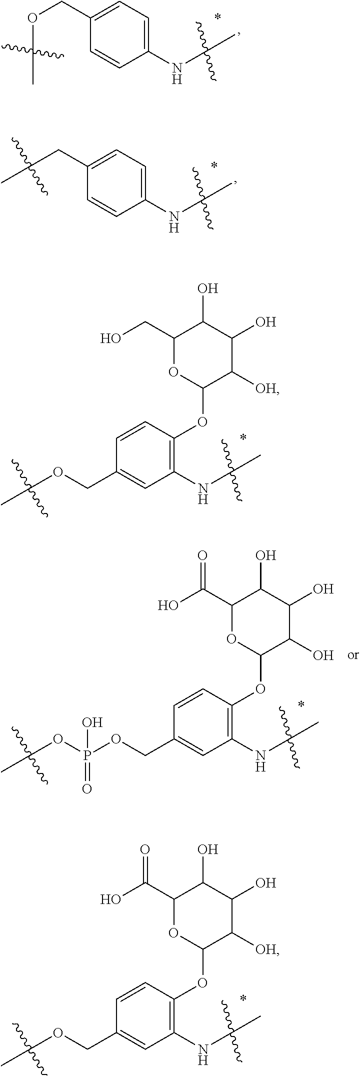 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

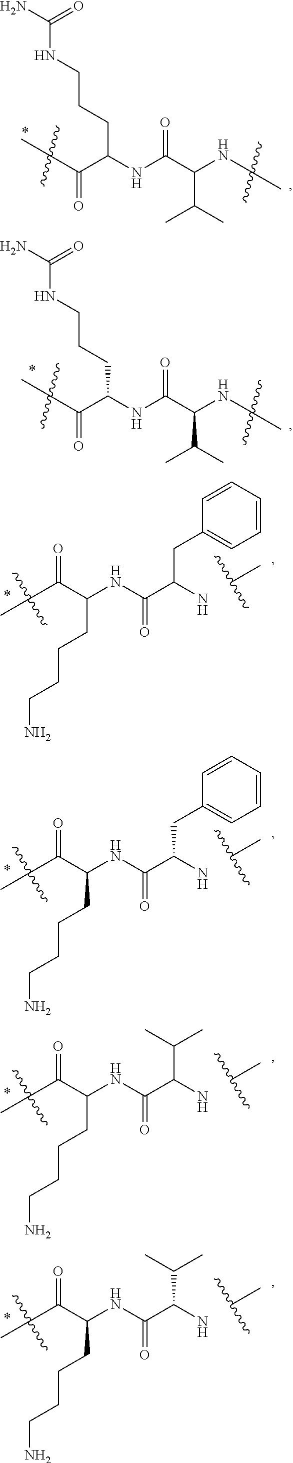

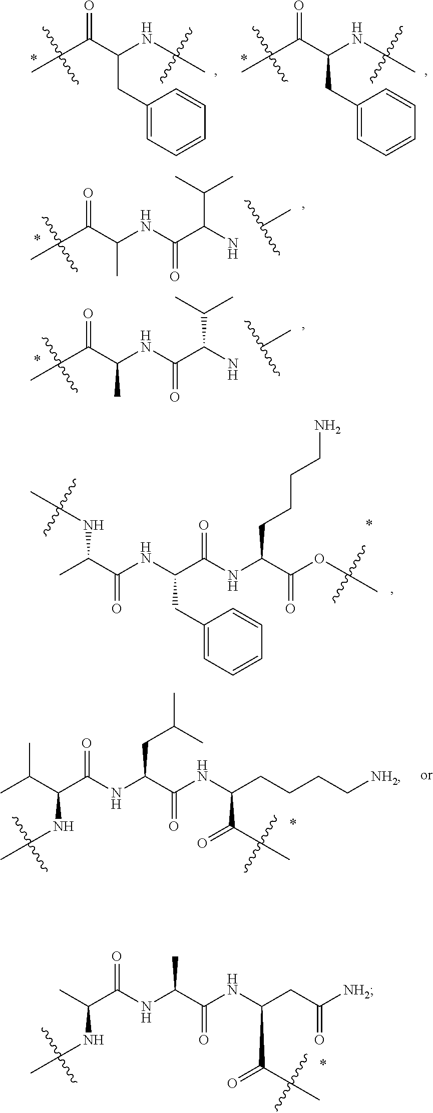

where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

Certain aspects and examples of the compounds of Formula (V) are provided in the following listing of additional, enumerated embodiments. It will be recognized that features specified in each embodiment may be combined with other specified features to provide further embodiments of the present invention.

Embodiment 136. The compound of Formula (V), wherein:
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—** or —C(═O)X₂X₃((CH₂)ₙO)ₜ(CH₂)ₙ—**, where the * denotes attachment point to R⁴.

Embodiment 137. The compound of Formula (V), wherein:
L is —C(═O)(CH₂)ₙ—**, —C(═O)((CH₂)ₙO)ₜ(CH₂)ₙ—**, —C(═O)X₂X₃C(═O)(CH₂)ₙ—** or —C(═O)X₂X₃((CH₂)ₙO)ₜ(CH₂)ₙ—**, where the * denotes attachment point to R⁴.

Embodiment 138. The compound of Formula (V), wherein:
L is —C(═O)(CH₂)ₙ—** or —C(═O)X₂X₃C(═O)(CH₂)ₙ—**, where the * denotes attachment point to R⁴.

Embodiment 139. The compound of Formula (V), wherein:
L is —C(═O)(CH₂)ₙ—**, where the * denotes attachment point to R⁴.

Embodiment 140. The compound of Formula (V), wherein:
L is —C(═O)(CH₂CH₂—**, where the * denotes attachment point to R⁴.

Embodiment 141. The compound of Formula (V), wherein:
R⁴⁰ is

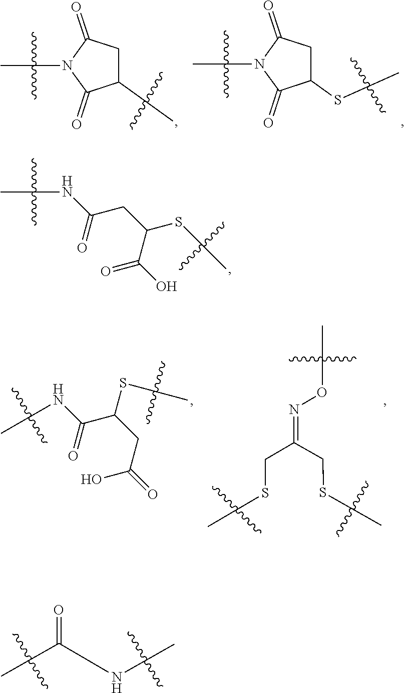 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NCC(*)=O]

### or —S—.

Embodiment 142. The compound of Formula (V), wherein: R⁴⁰ is

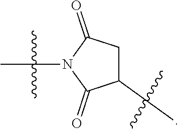 [SMILES: *C1CC(=O)N(*)C1=O]

Embodiment 143. The compound of Formula (V), wherein: R⁴⁰ is

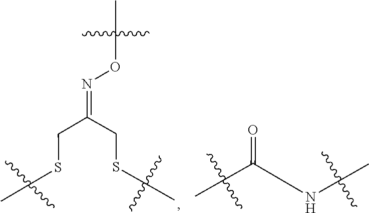 [SMILES: *ON=C(CS*)CS* ; *NCC(*)=O]

### or —S—.

Embodiment 144. The compound of Formula (V), wherein: R⁴⁰ is

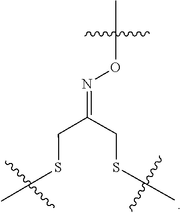 [SMILES: *ON=C(CS*)CS*]

Embodiment 145. The compound of Formula (V), wherein: R⁴⁰ is

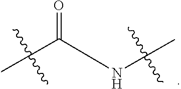 [SMILES: *NCC(*)=O]

Embodiment 146. The compound of Formula (V), wherein, X₁ is

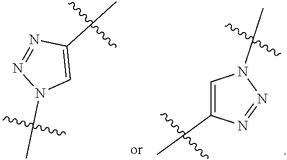 [SMILES: *c1cn(*)nn1 ; *c1cn(*)nn1]

Embodiment 147. The compound of Formula (V), wherein, X₁ is

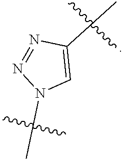 [SMILES: *c1cn(*)nn1]

Embodiment 148. The compound of Formula (V), wherein: X₂ is

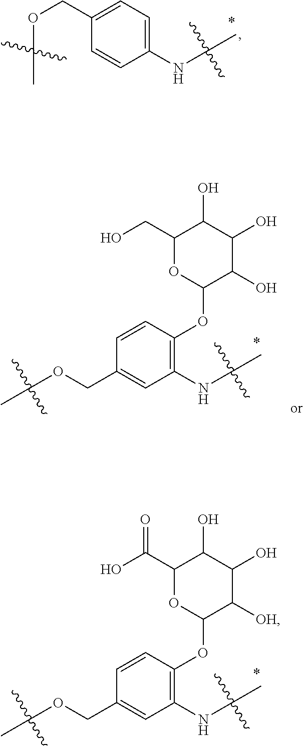 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃.

Embodiment 149. The compound of Formula (V), wherein: X₂ is

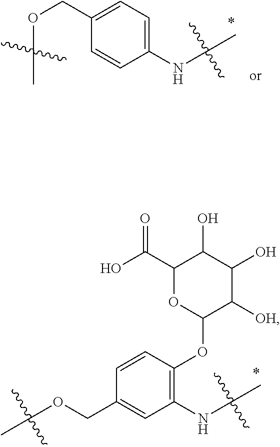 [SMILES: *Nc1ccc(CO*)cc1 ; *Nc1cc(CO*)ccc1OC1OC(C(=O)O)C(O)C(O)C1O]

where the * of X₂ indicates the point of attachment to X₃.

Embodiment 150. The compound of Formula (V), wherein, wherein: X₂ is

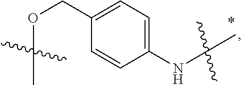 [SMILES: *Nc1ccc(CO*)cc1]

where the * of X₂ indicates the point of attachment to X₃.

Embodiment 151. The compound of Formula (V), wherein: X₂ is

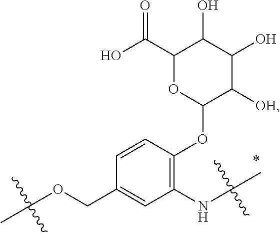 [SMILES: *Nc1cc(CO*)ccc1OC1OC(C(=O)O)C(O)C(O)C1O]

where the * of X₂ indicates the point of attachment to X₃.

Embodiment 152. The compound of Formula (V), wherein: X₃ is

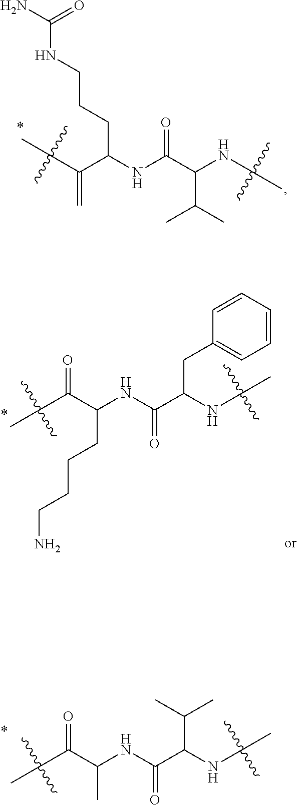 [SMILES: *NC(C(=O)NC(CCCNC(N)=O)C(*)=C)C(C)C ; *NC(Cc1ccccc1)C(=O)NC(CCCCN)C(*)=O ; *NC(C(=O)NC(C)C(*)=O)C(C)C]

where the * of X₃ indicates the point of attachment to X₂.

Embodiment 153. The compound of Formula (V), wherein: X₃ is

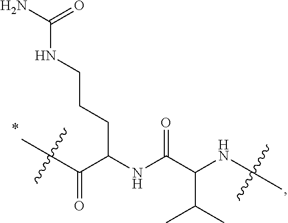 [SMILES: *NC(C(=O)NC(CCCNC(N)=O)C(*)=O)C(C)C]

where the * of X₃ indicates the point of attachment to X₂.

Embodiment 154. The compound of Formula (V), wherein: X₃ is

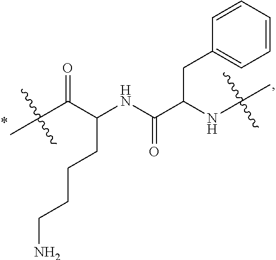 [SMILES: *NC(Cc1ccccc1)C(=O)NC(CCCCN)C(*)=O]

where the * of X₃ indicates the point of attachment to X₂.

Embodiment 155. The compound of Formula (V), wherein: X₃ is

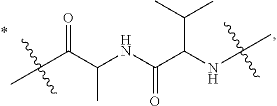 [SMILES: *NC(C(=O)NC(C)C(*)=O)C(C)C]

where the * of X₃ indicates the point of attachment to X₂.

Embodiment 156. The compound of Formula (V), wherein: R⁶ is 2-pyridyl or 4-pyridyl.

Embodiment 157. The compound of Formula (V), wherein: each R⁷ is independently selected from H and C₁-C₆alkyl.

Embodiment 158. The compound of Formula (V), wherein: each R⁷ is H.

Embodiment 159. The compound of Formula (V), wherein: each R⁷ is C₁-C₆alkyl.

Embodiment 160. The compound of Formula (V), wherein: each m is independently selected from 1, 2, 3, and 4.

Embodiment 161. The compound of Formula (V), wherein: each m is 1 or 2.

Embodiment 162. The compound of Formula (V), wherein: each n is independently selected from 1, 2, 3, and 4.

Embodiment 163. The compound of Formula (V), wherein: each n is 2 or 3.

Embodiment 164. The compound of Formula (V), wherein: each t is independently selected from 1, 2, 3, 4, 5, 6, 7, 8, 9, 10, 11, 12, 13, 14, 15, 16, 17 and 18.

Embodiment 165. The compound of Formula (V), wherein: each t is independently selected from 1, 2, 3, 4, 5 and 6.

Provided are also protocols for some aspects of analytical methodology for evaluating antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation overtime, adherence to surfaces, hepatotoxicity, clearance rates, and pharmacokinetic exposure. See Damle, N. K., Nat Biotechnol. 2008; 26(8):884-885; Singh, S. K., Pharm Res. 2015; 32(11):3541-71. When measured by hydrophobic interaction chromatography, higher hydrophobicity index scores (i.e. elution from HIC column faster) reflect lower hydrophobicity of the conjugates. In some embodiments, provided are antibody conjugates having a hydrophobicity index of 0.8 or greater, as determined by hydrophobic interaction chromatography.

### III) Drug Moieties

The antibody drug conjugates of the present application comprise DC-SIGN antibodies, antibody fragments thereof (e.g., antigen binding fragments), or functional equivalents that are conjugated to a drug moiety, e.g., an anti-cancer agent, an autoimmune treatment agent, an anti-inflammatory agent, an antifungal agent, an antibacterial agent, an anti-parasitic agent, an anti-viral agent, or an anesthetic agent. The antibodies, antibody fragments (e.g., antigen binding fragments), or functional equivalents of the invention can be conjugated to several identical or different drug moieties using any methods known in the art.

In certain embodiments, the drug moiety of the DC-SIGN antibody conjugates of the present invention is selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a CSF1R inhibitor, an A2AR inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an Eg5 inhibitor, proteasome inhibitors, an inhibitor of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor.

In one embodiment, the drug moiety of the DC-SIGN antibody conjugates of the present invention is a maytansinoid drug moiety, such as but not limited to, DM1, DM3, or DM4.

The antibodies, antibody fragments (e.g., antigen binding fragments) or functional equivalents of the present invention may be conjugated to a drug moiety that modifies a given biological response. Drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, Pseudomonas exotoxin, cholera toxin, or diphtheria toxin, a protein such as tumor necrosis factor, α-interferon, β-interferon, nerve growth factor, platelet derived growth factor, tissue plasminogen activator, a cytokine, an apoptotic agent, an anti-angiogenic agent, or, a biological response modifier such as, for example, a lymphokine. In some embodiments, the response modifier may be IL-10, TGFβ, JAK inhibitors, glucocorticoids, mTOR inhibitors, or vitamin D3 (1,25-dihydroxyvitamin D3).

In one embodiment, the antibodies, antibody fragments (e.g., antigen binding fragments) or functional equivalents of the present invention are conjugated to a drug moiety, such as a cytotoxin, a drug (e.g., an immunosuppressant) or a radiotoxin. Examples of cytotoxins include but are not limited to, taxanes (see, e.g., International (PCT) Patent Application Nos. WO 01/38318 and PCT/US03/02675), DNA-alkylating agents (e.g., CC-1065 analogs), anthracyclines, tubulysin analogs, duocarmycin analogs, auristatin E, auristatin F, maytansinoids, and cytotoxic agents comprising a reactive polyethylene glycol moiety (see, e.g., Sasse et al., J. Antibiot. (Tokyo), 53, 879-85 (2000), Suzawa et al., Bioorg. Med. Chem., 8, 2175-84 (2000), Ichimura et al., J. Antibiot. (Tokyo), 44, 1045-53 (1991), Francisco et al., Blood (2003) (electronic publication prior to print publication), U.S. Pat. Nos. 5,475,092, 6,340,701, 6,372,738, and 6,436,931, U.S. Patent Application Publication No. 2001/0036923 A1, Pending U.S. patent application Ser. Nos. 10/024,290 and 10/116,053, and International (PCT) Patent Application No. WO 01/49698), taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, anti-metabolites (e.g., methotrexate, 6-mercaptopurine, 6-thioguanine, cytarabine, 5-fluorouracil decarbazine), ablating agents (e.g., mechlorethamine, thiotepa chlorambucil, meiphalan, carmustine (BSNU) and Iomustine (CCNU), cyclophosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). (See e.g., Seattle Genetics US20090304721).

Other examples of cytotoxins that can be conjugated to the antibodies, antibody fragments (antigen binding fragments) or functional equivalents of the invention include duocarmycins, calicheamicins, maytansines and auristatins, and derivatives thereof.

Various types of cytotoxins, linkers and methods for conjugating therapeutic agents to antibodies are known in the art, see, e.g., Saito et al., (2003) Adv. Drug Deliv. Rev. 55:199-215; Trail et al., (2003) Cancer Immunol. Immunother. 52:328-337; Payne, (2003) Cancer Cell 3:207-212; Allen, (2002) Nat. Rev. Cancer 2:750-763; Pastan and Kreitman, (2002) Curr. Opin. Investig. Drugs 3:1089-1091; Senter and Springer, (2001) Adv. Drug Deliv. Rev. 53:247-264.

The antibodies, antibody fragments (e.g., antigen binding fragments) or functional equivalents of the present invention can also be conjugated to a radioactive isotope to generate cytotoxic radiopharmaceuticals, referred to as radioimmunoconjugates. Examples of radioactive isotopes that can be conjugated to antibodies for use diagnostically or therapeutically include, but are not limited to, iodine-131, indium-111, yttrium-90, and lutetium-177. Methods for preparing radioimmunoconjugates are established in the art. Examples of radioimmunoconjugates are commercially available, including Zevalin™ (DEC Pharmaceuticals) and Bexxar™ (Corixa Pharmaceuticals), and similar methods can be used to prepare radioimmunoconjugates using the antibodies of the invention. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., (1998) Clin Cancer Res. 4(10):2483-90; Peterson et al., (1999) Bioconjug. Chem. 10(4):553-7; and Zimmerman et al., (1999) Nucl. Med. Biol. 26(8):943-50, each incorporated by reference in their entireties.

The antibodies, antibody fragments (e.g., antigen binding fragments) or functional equivalents of the present invention can also conjugated to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antibody fragment (e.g., antigen binding fragment) described herein (e.g., a Fab fragment, Fd fragment, Fv fragment, F(ab)₂ fragment, a VH domain, a VH CDR, a VL domain or a VL CDR) and a heterologous protein, polypeptide, or peptide.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., (1997) Curr. Opinion Biotechnol. 8:724-33; Harayama, (1998) Trends Biotechnol. 16(2):76-82; Hansson et al., (1999) J. Mol. Biol. 287:265-76; and Lorenzo and Blasco, (1998) Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an antigen may be recombined with one or more components, motifs, sections, parts, domains, fragments, etc. of one or more heterologous molecules.

Moreover, the antibodies, antibody fragments (e.g., antigen binding fragments) or functional equivalents of the present invention can be conjugated to marker sequences, such as a peptide, to facilitate purification. In preferred embodiments, the marker amino acid sequence is a hexa-histidine peptide (SEQ ID NO: 928), such as the tag provided in a pQE vector (QIAGEN, Inc., 9259 Eton Avenue, Chatsworth, Calif., 91311), among others, many of which are commercially available. As described in Gentz et al., (1989) Proc. Natl. Acad. Sci. USA 86:821-824, for instance, hexa-histidine (SEQ ID NO: 928) provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “FLAG” tag (A. Einhauer et al., J. Biochem. Biophys. Methods 49: 455-465, 2001). According to the present invention, antibodies or antigen binding fragments can also be conjugated to tumor-penetrating peptides in order to enhance their efficacy.

In other embodiments, the antibodies, antibody fragments (e.g., antigen binding fragments) or functional equivalents of the present invention are conjugated to a diagnostic or detectable agent. Such immunoconjugates can be useful for monitoring or prognosing the onset, development, progression and/or severity of a disease or disorder as part of a clinical testing procedure, such as determining the efficacy of a particular therapy. Such diagnosis and detection can be accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, umbelliferone, fluorescein, fluorescein isothiocyanate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 64Cu, 113Sn, and 117Sn; and positron emitting metals using various positron emission tomographies, and non-radioactive paramagnetic metal ions.

The antibodies, antibody fragments (e.g., antigen binding fragments) or functional equivalents of the invention may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### IV) DC-SIGN Antibody Fusion Proteins

The present invention provides antibody fusion proteins, where an antibody, or fragment thereof (e.g. antigen binding fragment), or its functional equivalent that specifically binds to DC-SIGN is fused to a polypeptide, such as a peptide antigen. Without wishing to be bound by any particular theory, the peptide antigen should be capable of binding to a MHC class II or MHC Class I molecule, or should be capable of being processed within an antigen-presenting cell (such as a dendritic cell) to give rise to one or more peptides capable of binding to a MHC class II molecule or MHC Class I. It has recently been suggested that short epitope peptides of around 8 amino acids in length may induce less sustained CTL reactivity than longer peptides (e.g. around 30 amino acids in length). MHC class I molecules typically bind peptides of 8 or 9 amino acids in length, while MHC class II molecules can bind peptides from 8 amino acids up to amino acids, up 30 amino acids, or even longer.

The antigen may be any protein or fragment thereof against which it is desirable to raise an immune response, in particular a CTL response, but also a Th17 response or a Treg response. These may include antigens associated with, expressed by, displayed on, or secreted by cells against which it is desirable to stimulate a CTL response, including cancer cells and cells containing intracellular pathogens or parasites. For example, the antigen may be, or may comprise, an epitope peptide from a protein expressed by an intracellular pathogen or parasite (such as a viral protein) or from a protein expressed by a cancer or tumour cell. Thus the antigen may be a tumour-specific antigen. The term “tumour-specific” antigen should not be interpreted as being restricted to antigens from solid tumours, but to encompass antigens expressed specifically by any cancerous, transformed or malignant cell.

It may be particularly desirable to raise a Treg response against an antigen to which the subject exhibits, or is at risk of developing, an undesirable immune response. For example, it may be a self antigen against which an immune response occurs in an autoimmune disease. Examples of autoimmune diseases in which specific antigens have been identified as potentially pathogenically significant include multiple sclerosis (myelin basic protein), insulin-dependent diabetes mellitus (glutamic acid decarboxylase), insulin-resistant diabetes mellitus (insulin receptor), coeliac disease (gliadin), bullous pemphigoid (collagen type XVII), auto-immune haemolytic anaemia (Rh protein), auto-immune thrombocytopenia (GpIIb/IIIa), myaesthenia gravis (acetylcholine receptor), Graves' disease (thyroid-stimulating hormone receptor), glomerulonephritis, such as Goodpasture's disease (alpha3(IV)NC1 collagen), and pernicious anaemia (intrinsic factor). Alternatively the target antigen may be an exogenous antigen which stimulates a response which also causes damage to host tissues. For example, acute rheumatic fever is caused by an antibody response to a Streptococcal antigen which cross-reacts with a cardiac muscle cell antigen. Thus these antigens, or particular fragments or epitopes thereof may be suitable antigens for use in the present invention.

Depletion of Treg cells or impairment of Treg cell function has been shown to result in autoimmune disease in murine models. Disease caused in test animals include arthritis (e.g. rheumatoid arthritis), inflammatory bowel disease, gastritis, pernicious anaemia, thyroiditis, insulitis, diabetes, sialoadenitis, adrenalitis, autoimmune orchitis/oophoritis, glomerulonephritis, chronic obstructive pulmonary disease and experimental autoimmune encephalitis and multiple sclerosis.

Induction of a regulatory T cell type 1 response has also been shown to reduce the development of atherosclerosis in murine models (Mallat Z. et al. Circulation 108:1232-7, 2003).

Treg activity has also been shown to be significant in the rate at which allografts are rejected. Depletion of Treg cells or impairment of function accelerates the rate of rejection, while infusion of test animals with syngeneic lymphocytes enriched in Treg cells has been shown to prolong graft survival.

In one aspect, the antibodies, antigen binding fragments, or their functional equivalents of the invention are fused to the polypeptide, such as a peptide antigen, with or without a linker. In some embodiments, the linker may be a peptide linker. In certain embodiments one or more peptide linkers is independently selected from a (Glyₙ-Ser)ₘ sequence (SEQ ID NO: 929), a (Glyₙ-Ala)ₘsequence (SEQ ID NO: 930), or any combination of a (Glyₙ-Ser)ₘ/(Glyₙ-Ala)ₘ sequence, wherein each n is independently an integer from 1 to 5 and each m is independently an integer from 0 to 10. In some embodiments a peptide linker is (Gly₄-Ser)ₘwherein m is an integer from 0 to 10 (SEQ ID NO: 931). In some embodiments a peptide linker is (Gly₄-Ala)ₘwherein m is an integer from 0 to 10 (SEQ ID NO: 932). Examples of linkers include, but are not limited to, certain embodiments one or more linkers include G₄S (SEQ ID NO: 332) repeats, e.g., the Gly-Ser linker GGGGS (SEQ ID NO:332), or (GGGGS)ₘ wherein m is a positive integer equal to or greater than 1 (SEQ ID NO: 332). For example, m=1, m=2, m=3. m=4, m=5 and m=6, m=7, m=8, m=9 and m=10. In some embodiments, the linker includes multiple repeats of GGGGS (SEQ ID NO:332), including, but is not limited to (GGGGS)₃ (SEQ ID NO: 933) or (GGGGS)₄ (SEQ ID NO: 934). In some embodiments, Ser can be replaced with Ala e.g., linkers G/A such as (GGGGA) (SEQ ID NO: 333), or (GGGGA)ₘ wherein m is a positive integer equal to or greater than 1 (SEQ ID NO: 935). In some embodiments, the linker includes multiple repeats of GGGGA (SEQ ID NO:333). In other embodiments, a linker includes combinations and multiples of GGGGS (SEQ ID NO:332) and GGGGA (SEQ ID NO:333). In some embodiments, polypeptide, such as a peptide antigen, may be fused to the N-terminus, C-terminus, or an internal site of a peptide chain, e.g., heavy chain or light chain, of the antibody, antigen binding fragment thereof, or its functional equivalent. In some embodiments, polypeptide, such as a peptide antigen, may be fused to a CDR of the antibody, antigen binding fragment thereof, or its functional equivalent.

In some embodiments, the antibody, or fragment thereof (e.g. antigen binding fragment), or its functional equivalent that specifically binds to DC-SIGN is further linked to a drug moiety, such as an immunostimulatory molecule, as disclosed herein. Without wishing to be bound by theory, the DC-SIGN immunoconjugate-antigen fusion proteins may be capable of enhancing an immune response against the peptide antigen due to the activation of DC-SIGN expressing antigen presenting cells such as DCs or macrophages.

### Immunostimulatory Molecules of the Invention

This application provides immunomodulatory molecules. In some embodiments, the immunomodulatory molecules or immunomodulators are immunostimulatory molecules, i.e. compounds that enhance activity of the immune system, wherein the immunostimulatory molecules are not STING agonists. The immunostimulatory molecules provided herein can be a small molecule compound, a nucleic acid molecule, a polypeptide, or a combination thereof. In some embodiments, the immunostimulatory molecules disclosed herein is a dendritic cell stimulating compound, for example, a DEC-205 agonist, FLT3 ligand, granulocyte macrophage colony-stimulating factor (GM-CSF), an agonist of a Toll-like receptor (TLR) (e.g., TLR1, TLR2, TLR3, TLR4, TLR5, TLR6, TLR7, TLR8, TLR9, TLR10), RIG-I, MDA-5, LGP2, a C-type lectin receptor agonist, NOD1, NOD2, costimulatory compounds such as IL-15 or agonists of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In some embodiments, the immunostimulatory molecules of the invention are TLR7 agonists having the structure of Formula (I):

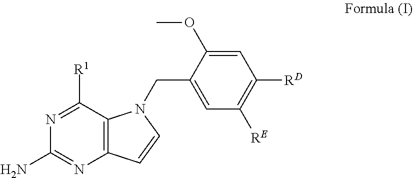

wherein:
R^{D} is

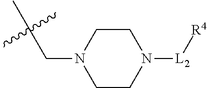

and R^{E} is H; or R^{E} is

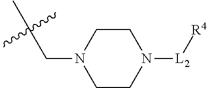

### and R^{D} is H;

R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ**-, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴;
R⁴ is

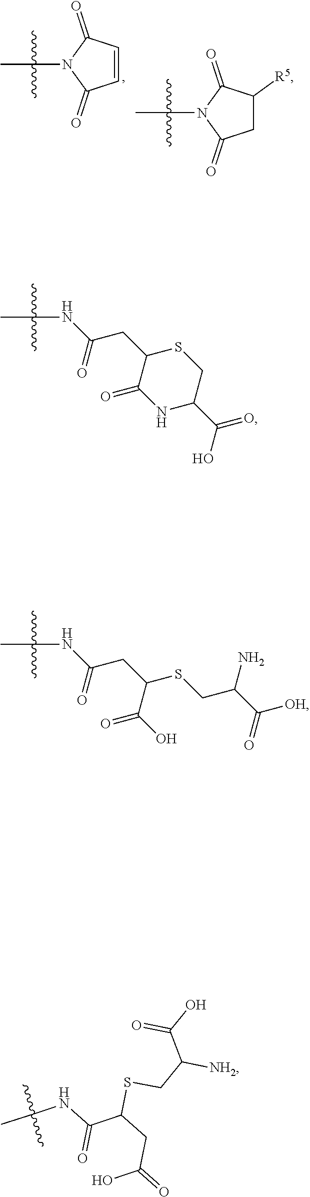

—ONH₂, —NH₂,

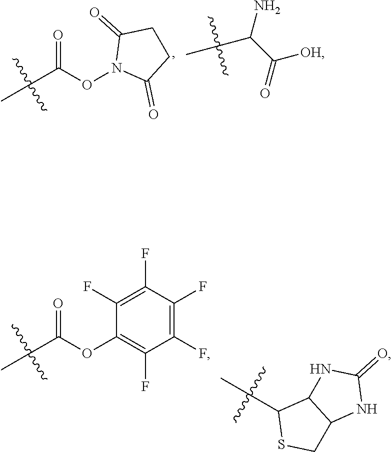 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

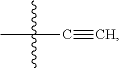 [SMILES: *C#C]

NHC(═O)CH═CH₂, —SH, —SR⁷, —OH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

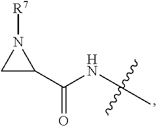

—CO₂H, —C(O)NHNH₂,

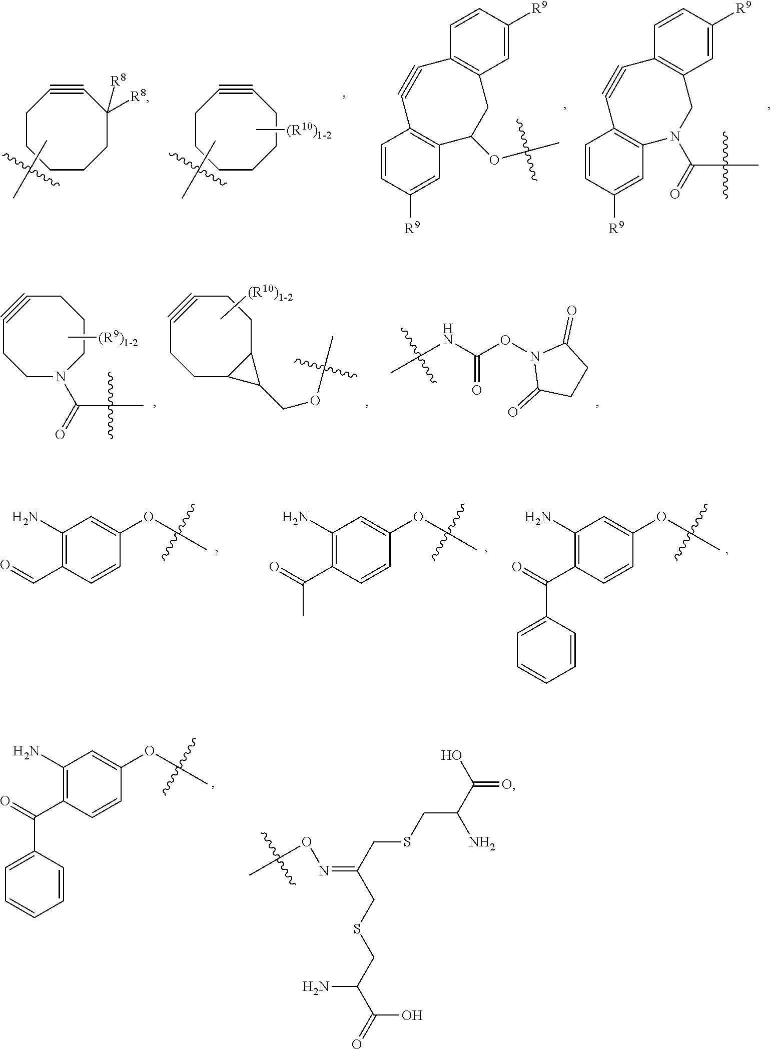

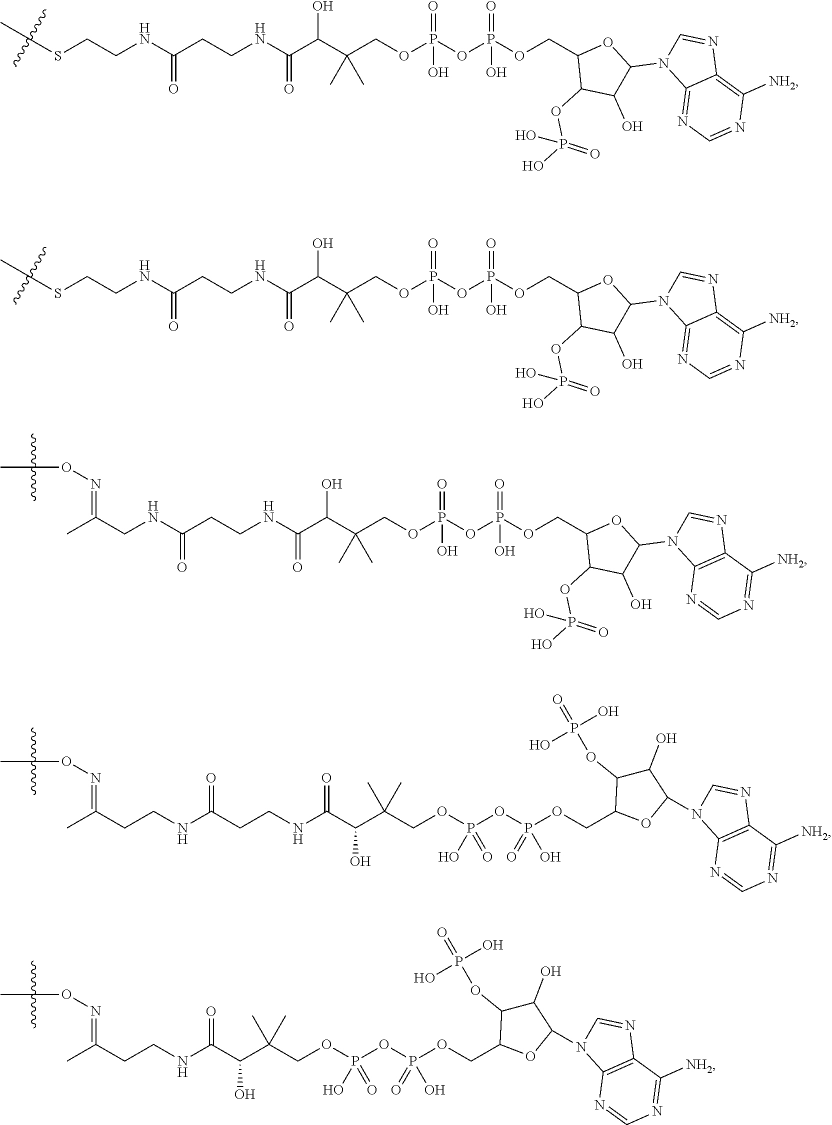 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CCNC(=O)CCNC(=O)[C@@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@@H](O)C(C)(C)COP(=O)(O)OP(=O)(O)OCC1OC(n2cnc3c(N)ncnc32)C(O)C1OP(=O)(O)O]

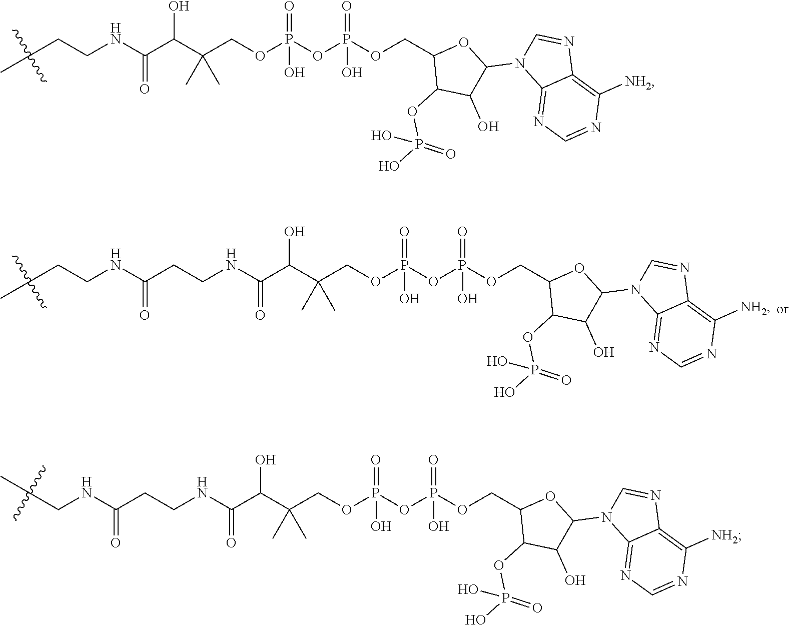 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

R⁵ is

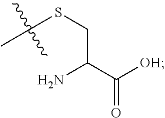 [SMILES: *SCC(N)C(=O)O]

X₁ is

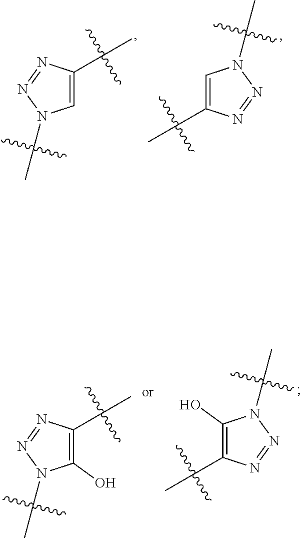 [SMILES: *c1cn(*)nn1 ; *c1cn(*)nn1 ; *c1nnn(*)c1O ; *c1nnn(*)c1O]

X₂ is

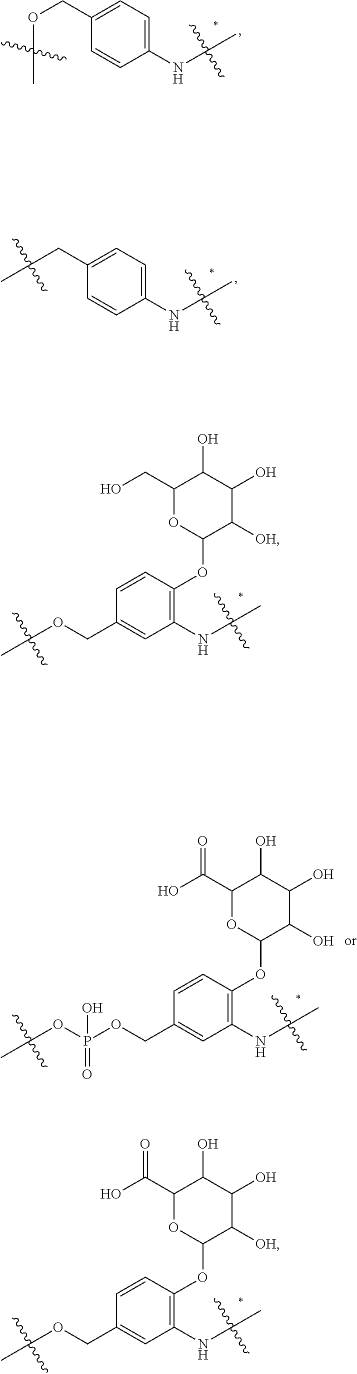 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

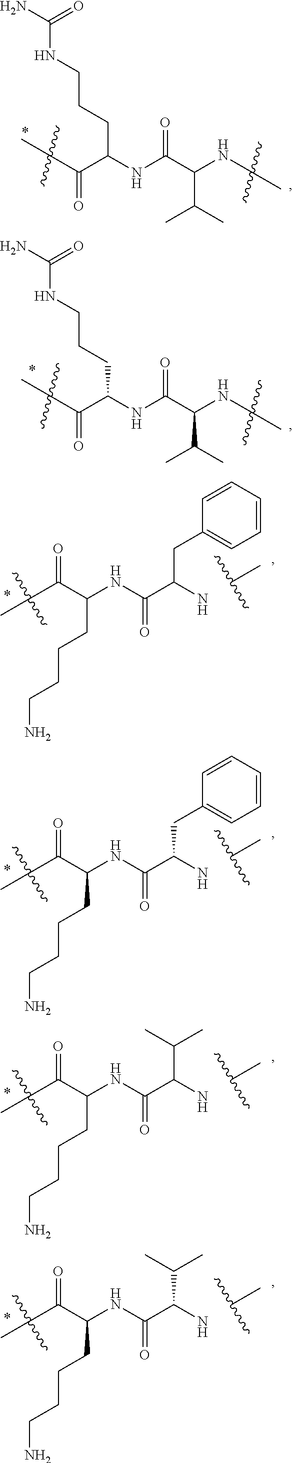

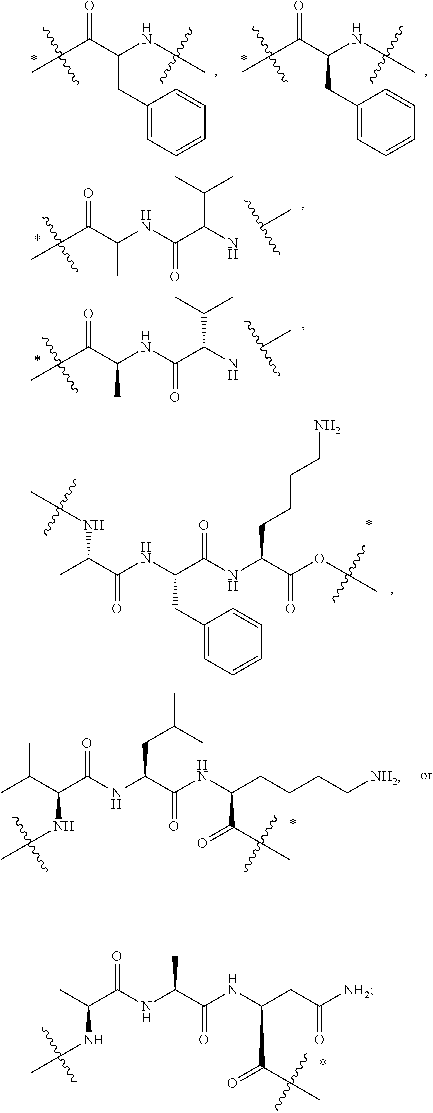

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1,2,3, and 4; and
each t is independently selected from 1, 2, 3, 4, 5, 6, 7, 8, 9, 10, 11, 12, 13, 14, 15, 16, 17 and 18.

Certain aspects and examples of the compounds of Formula (I) are provided in the following listing of additional, enumerated embodiments. It will be recognized that features specified in each embodiment may be combined with other specified features to provide further embodiments of the present invention.

Embodiment 1. The compound of Formula (I), and the pharmaceutically acceptable salts thereof, wherein:
R^{D} is

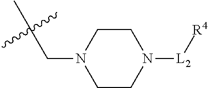

and R^{E} is H; or R^{E} is

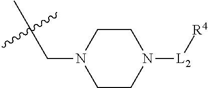

### and R^{D} is H;

R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ**-, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴;
R⁴ is

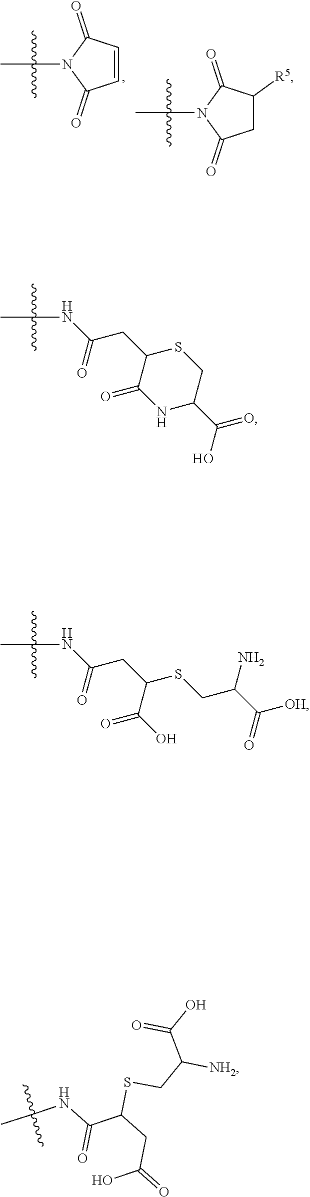

—ONH₂, —NH₂,

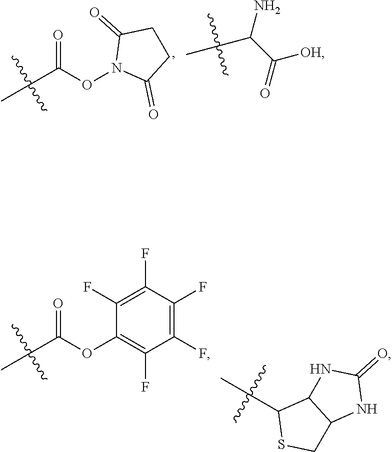 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

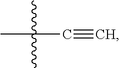 [SMILES: *C#C]

—NHC(═O)CH═CH₂, SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

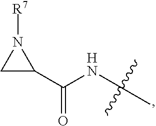

—CO₂H, —C(O)NHNH₂,

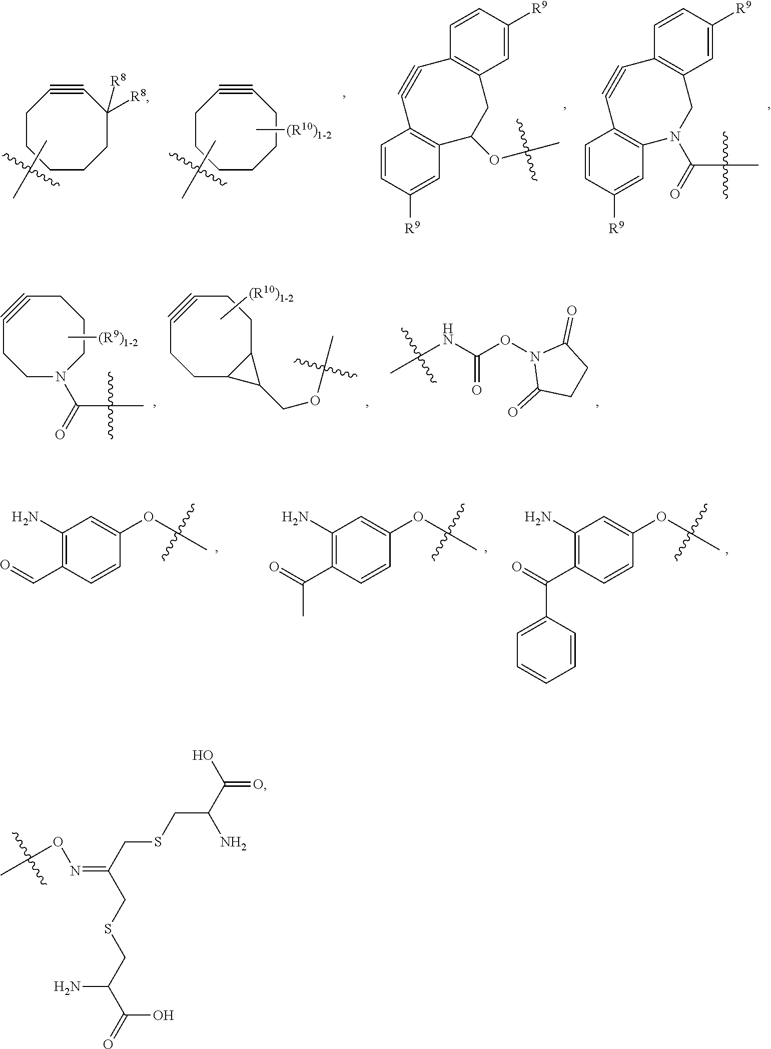

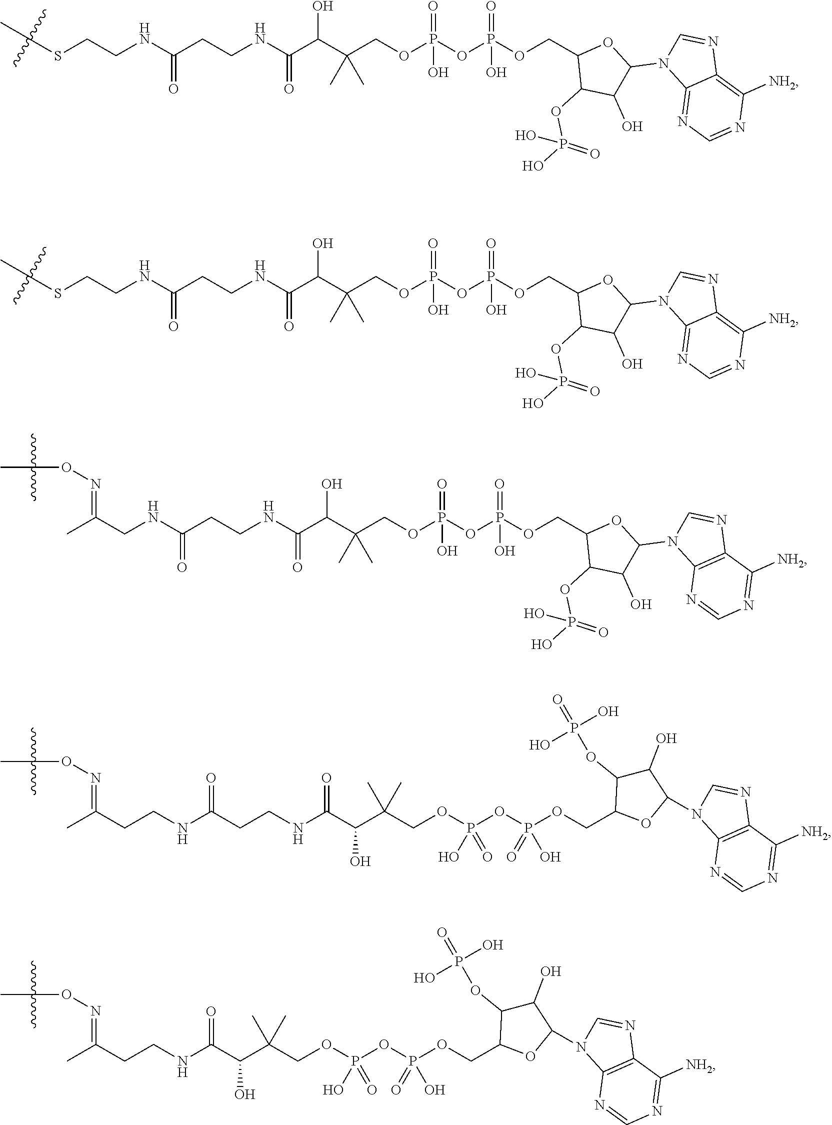 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CCNC(=O)CCNC(=O)[C@@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@@H](O)C(C)(C)COP(=O)(O)OP(=O)(O)OCC1OC(n2cnc3c(N)ncnc32)C(O)C1OP(=O)(O)O]

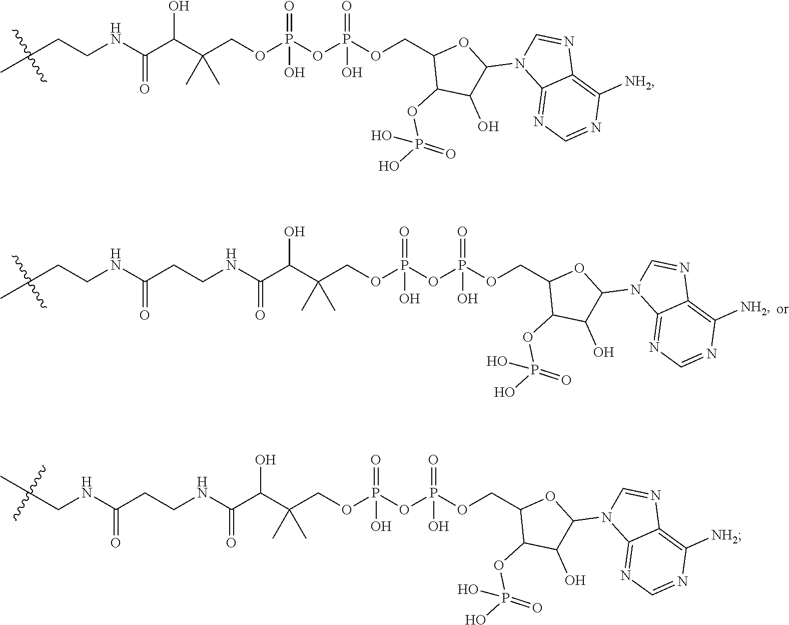 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

R⁵ is

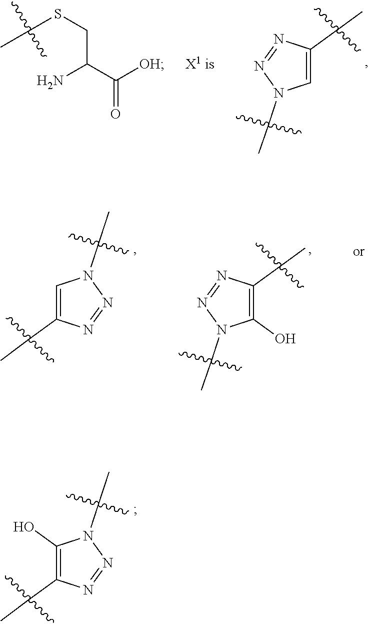 [SMILES: *SCC(N)C(=O)O ; *c1cn(*)nn1 ; *c1cn(*)nn1 ; *c1nnn(*)c1O ; *c1nnn(*)c1O]

X₂ is

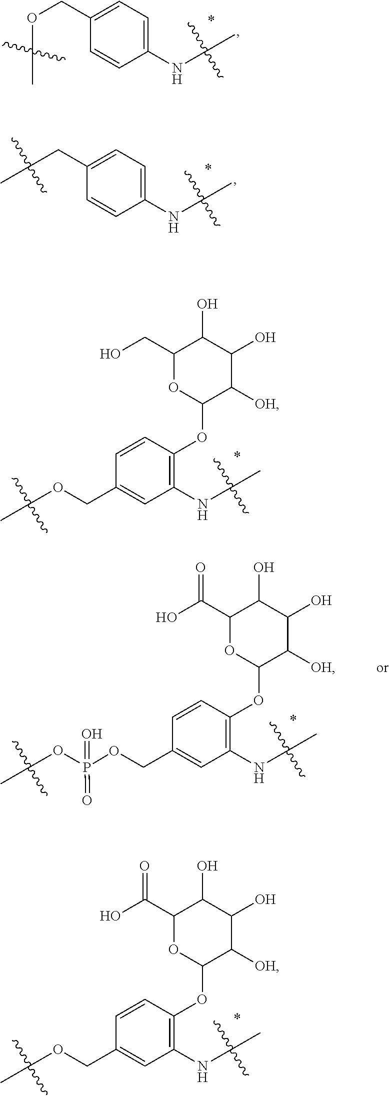

where the * of X₂ indicates the point of attachment to X₃;
X₃ is

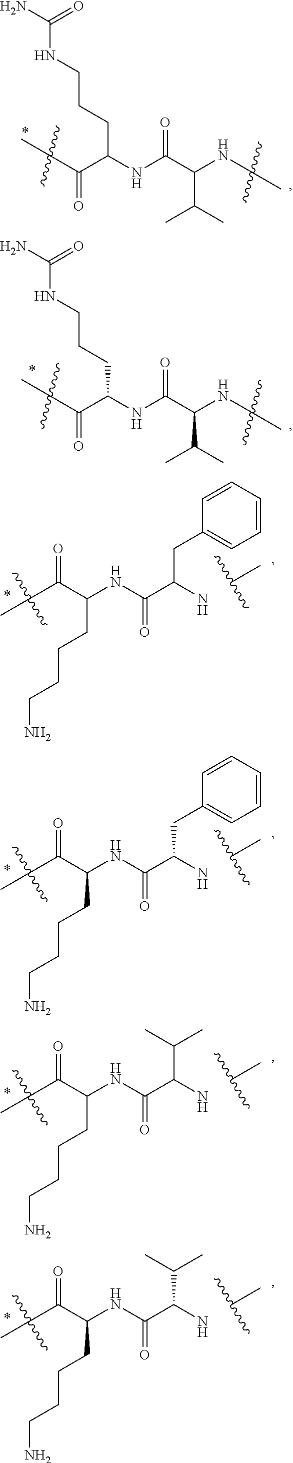

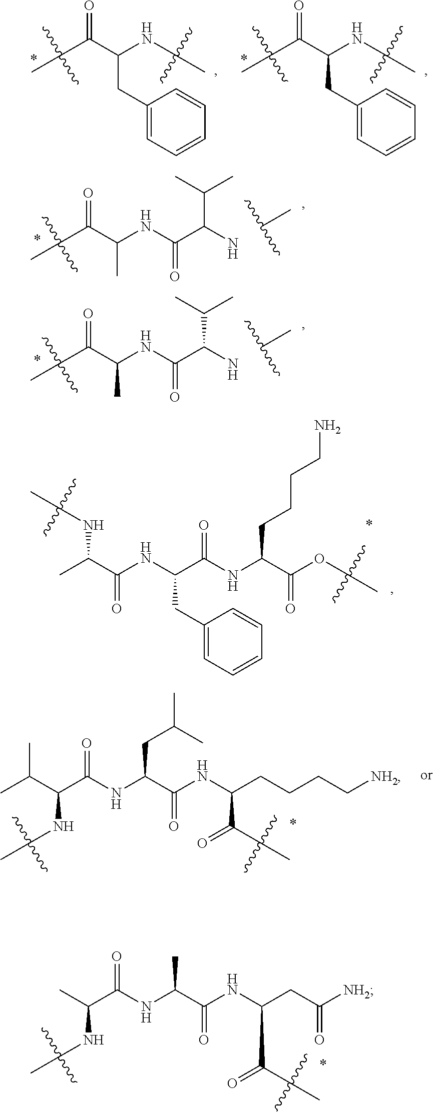

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and
   each t is independently selected from 1, 2, 3, 4, 5, 6, 7, 8, 9, 10, 11, 12, 13, 14, 15, 16, 17 and 18.

Embodiment 2. The compound of Formula (I) having the structure of Formula (Ia) or Formula (Ib), and the pharmaceutically acceptable salts thereof:

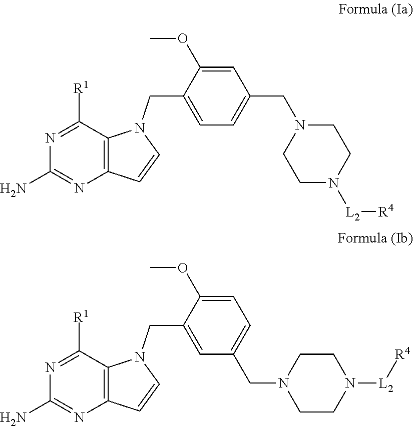

wherein:
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂X₃C(═O)(CH₂)ₙ—**, —C(═O)X₂C(═O)(CH₂)ₙNHC(═O)(CH₂)ₙ—**, —C(═O)X₂C(═O)(CH₂)ₙNHC(═O)((CH₂)ₙO)ₜ(CH₂)ₙ—**, —C(═O)(CH₂)ₙC(R₇)₂—**, —C(═O)(CH₂)ₙC(R₇)₂SS(CH₂)ₙNHC(═O)(CH₂)ₙ—**, —(CH₂)ₙX₂C(═O)(CH₂)ₙNHC(═O)((CH₂)ₙO)ₜ(CH₂)ₙ—** or —C(═O)(CH₂)ₙC(═O)NH(CH₂)ₙ—**, where the ** of L₂ indicates the point of attachment to R⁴;
R⁴ is

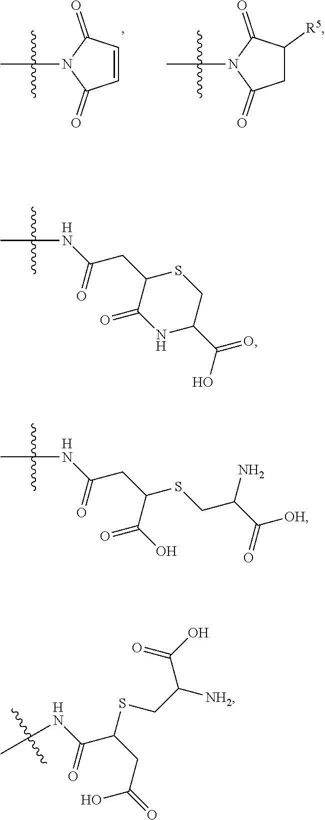

—ONH₂, —NH₂,

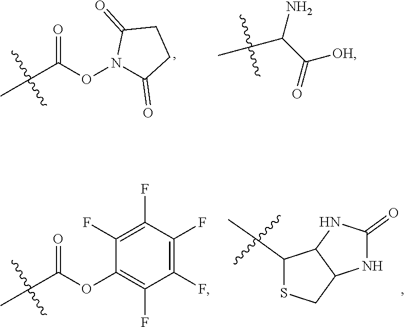 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

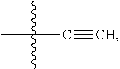 [SMILES: *C#C]

—NHC(═O)CH═CH₂, SH, —SR⁷, —OH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

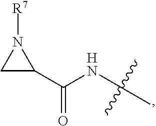

—CO₂H, —C(O)NHNH₂,

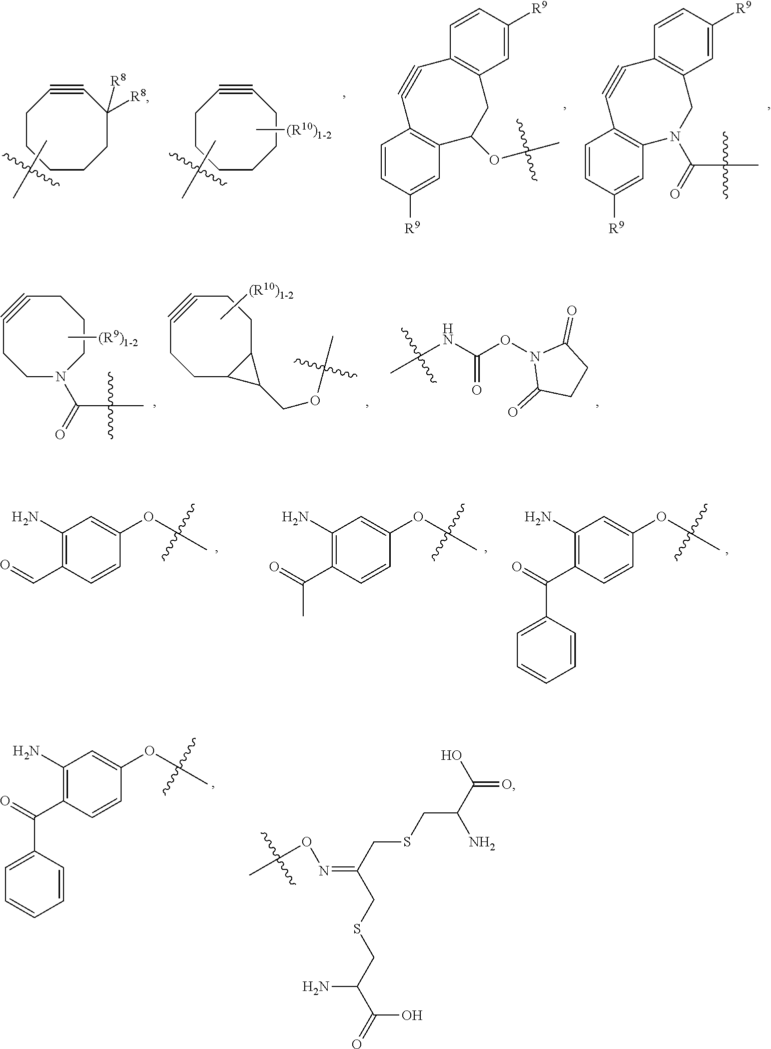

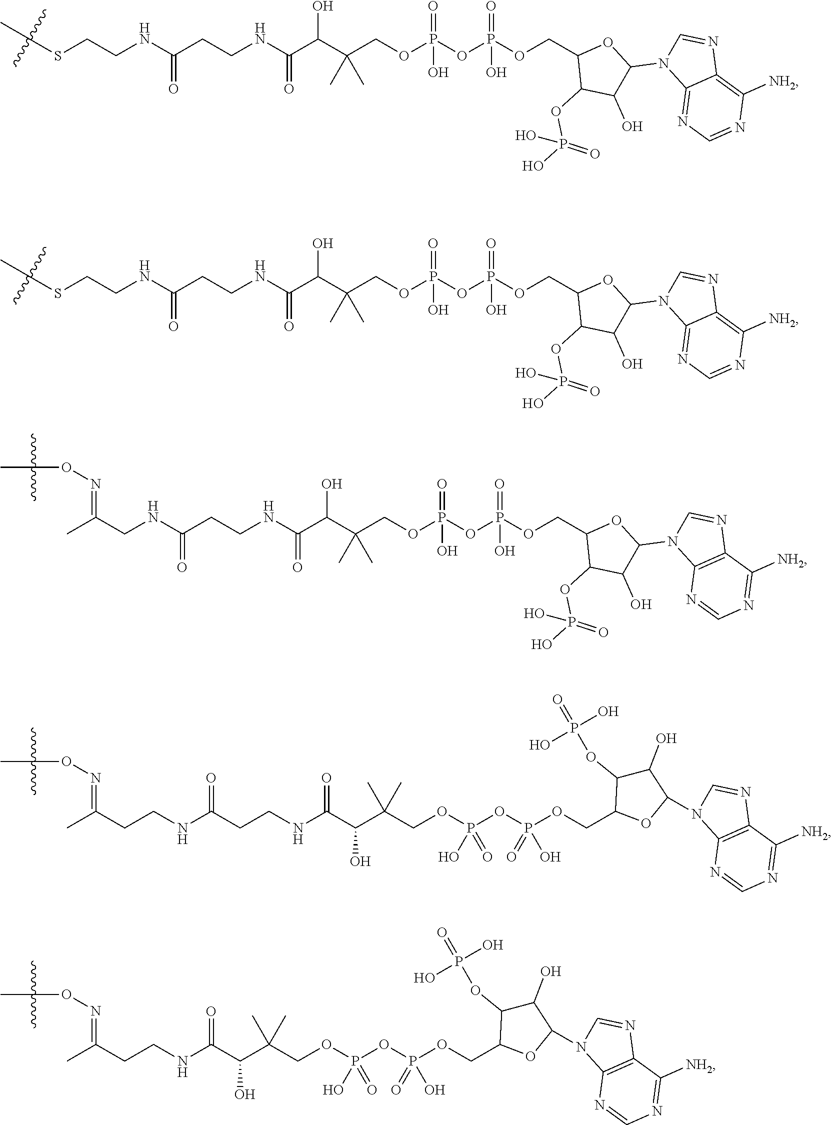 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CCNC(=O)CCNC(=O)[C@@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@@H](O)C(C)(C)COP(=O)(O)OP(=O)(O)OCC1OC(n2cnc3c(N)ncnc32)C(O)C1OP(=O)(O)O]

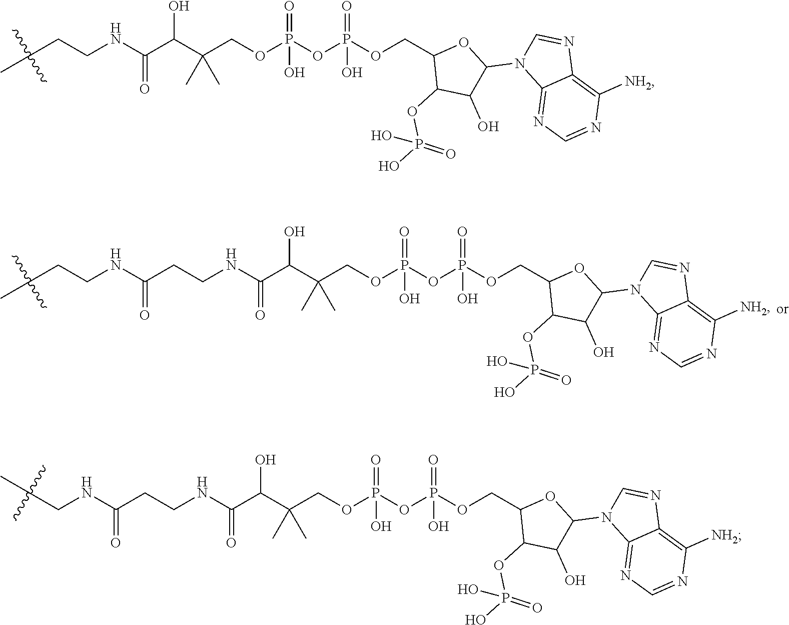 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

R⁵ is

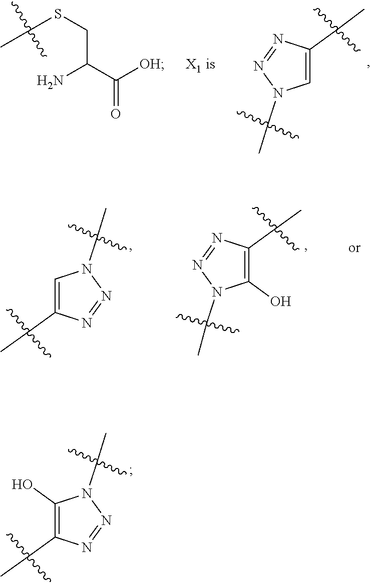 [SMILES: *SCC(N)C(=O)O ; *c1cn(*)nn1 ; *c1cn(*)nn1 ; *c1nnn(*)c1O ; *c1nnn(*)c1O]

X₂ is

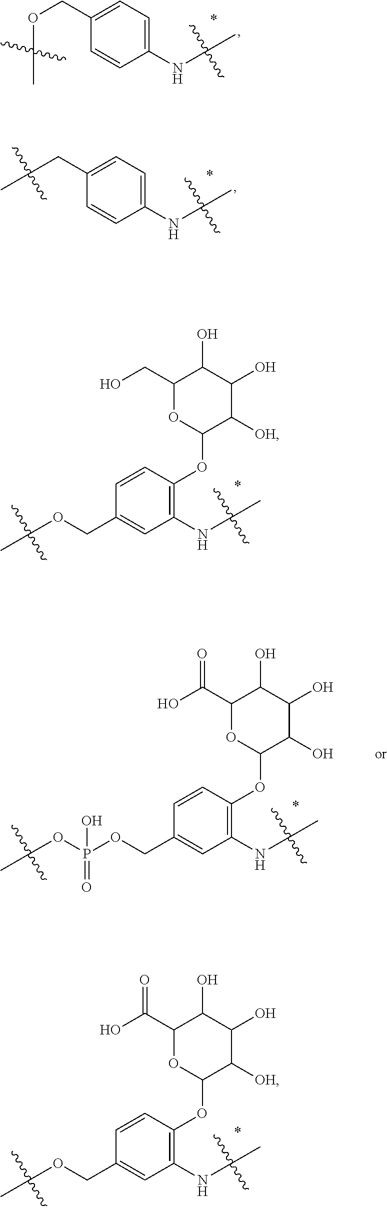 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

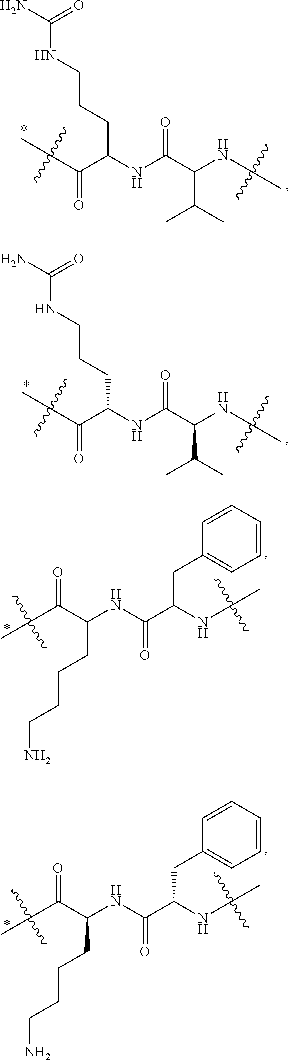

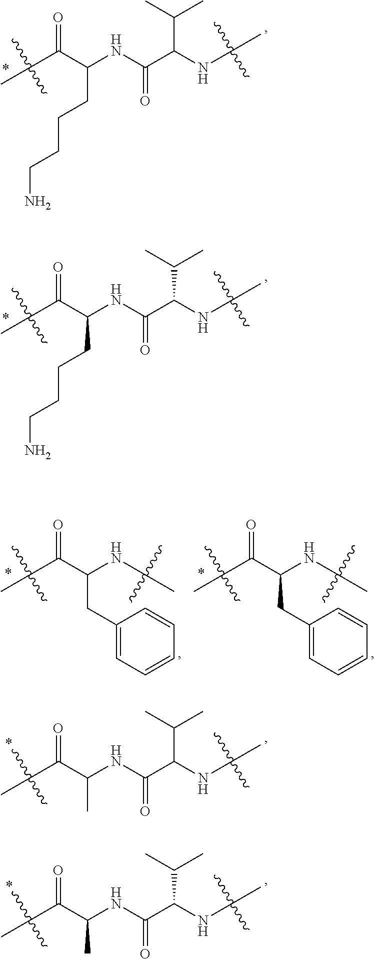

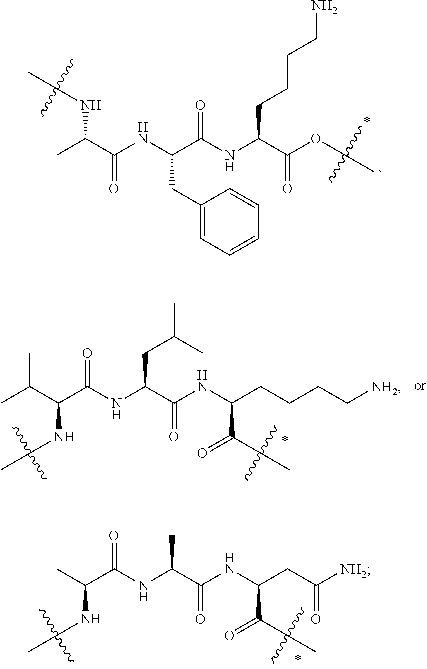 [SMILES: *N[C@@H](C)C(=O)N[C@@H](Cc1ccccc1)C(=O)N[C@@H](CCCCN)C(=O)O* ; *N[C@H](C(=O)N[C@@H](CC(C)C)C(=O)N[C@@H](CCCCN)C(*)=O)C(C)C ; *N[C@@H](C)C(=O)N[C@@H](C)C(=O)N[C@@H](CC(N)=O)C(*)=O]

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and

each t is independently selected from 1, 2, 3, 4, 5, 6, 7, 8, 9, 10, 11, 12, 13, 14, 15, 16, 17 and 18.

Embodiment 3. The compound of Formula (Ia) or Formula (Ib), and the pharmaceutically acceptable salts thereof, wherein:
R¹ is —NHR₂ or —NHCHR₂R₃;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴;
R⁴ is

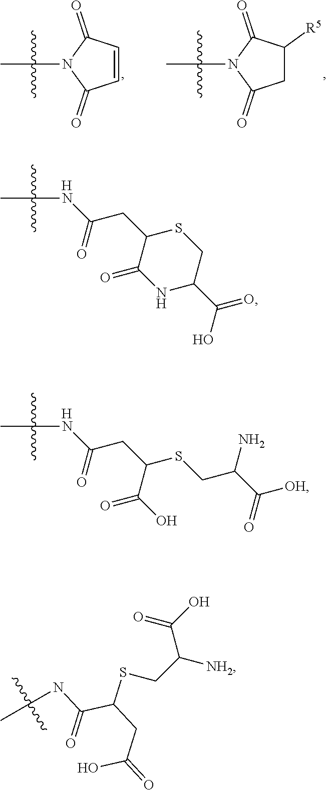

—ONH₂, —NH₂,

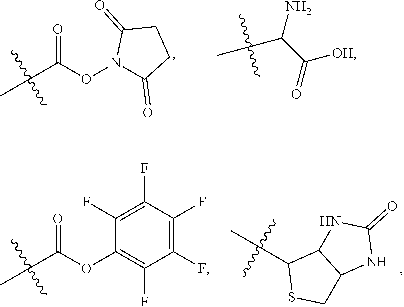 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

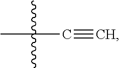 [SMILES: *C#C]

—NHC(═O)CH═CH₂, SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

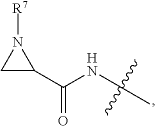

—CO₂H, —C(O)NHNH₂,

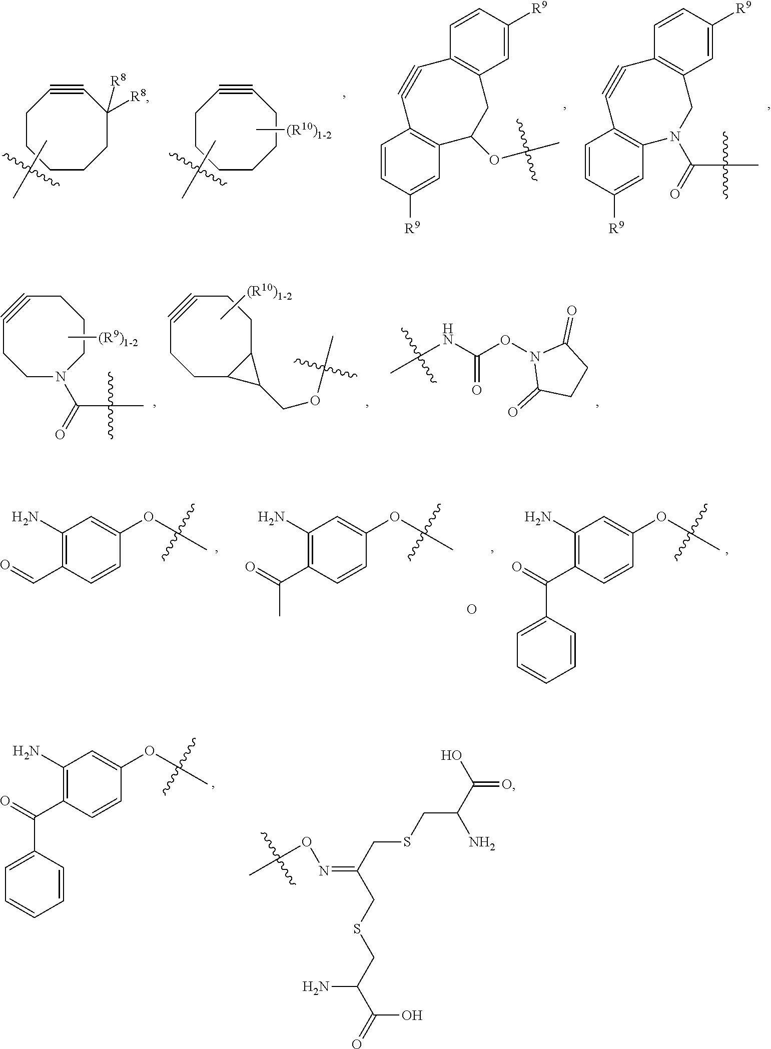

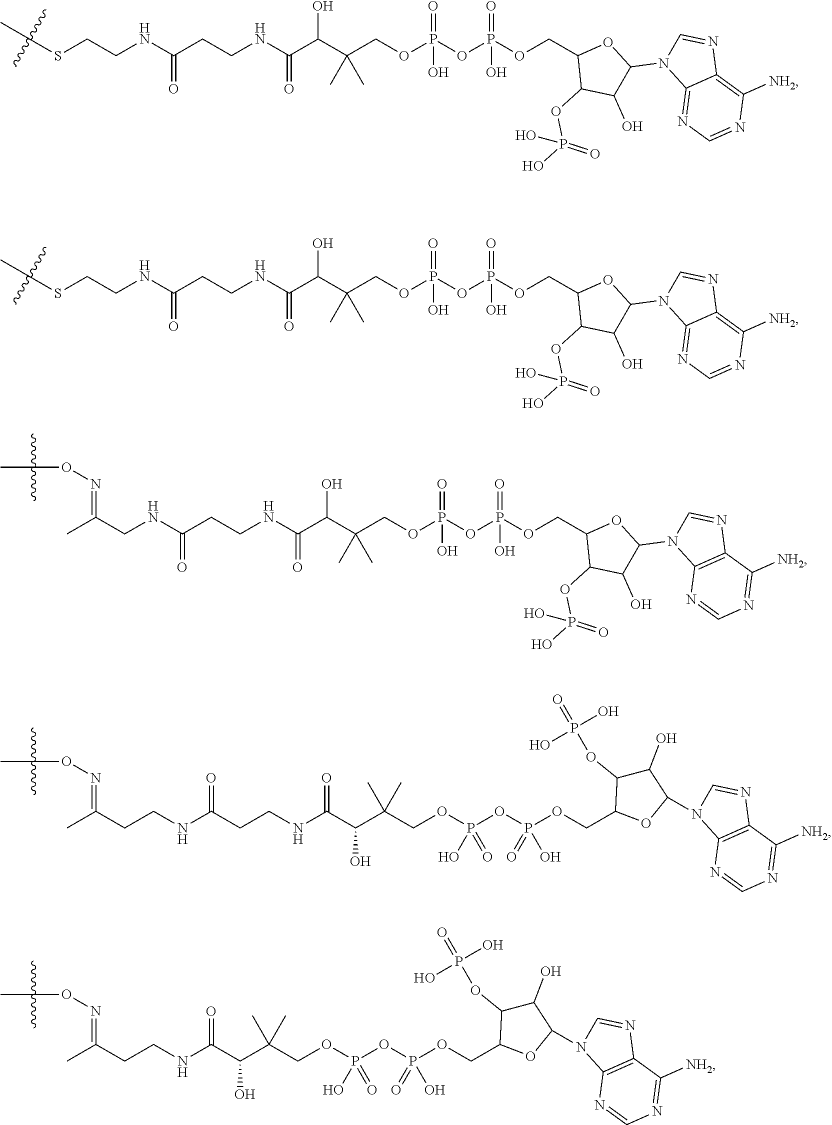 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CCNC(=O)CCNC(=O)[C@@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@@H](O)C(C)(C)COP(=O)(O)OP(=O)(O)OCC1OC(n2cnc3c(N)ncnc32)C(O)C1OP(=O)(O)O]

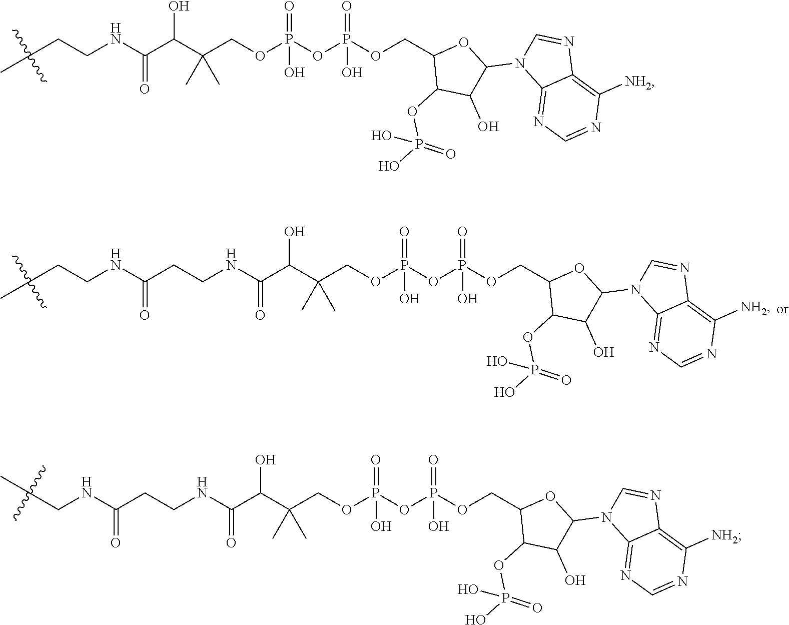 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

R⁵ is

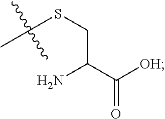 [SMILES: *SCC(N)C(=O)O]

X₁ is

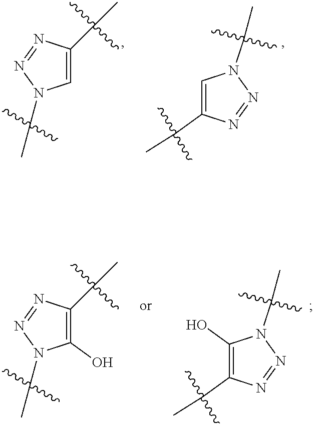 [SMILES: *c1cn(*)nn1 ; *c1cn(*)nn1 ; *c1nnn(*)c1O ; *c1nnn(*)c1O]

X₂ is

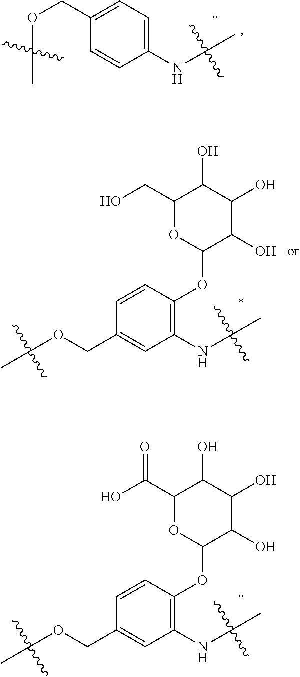 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃;
X₃ is

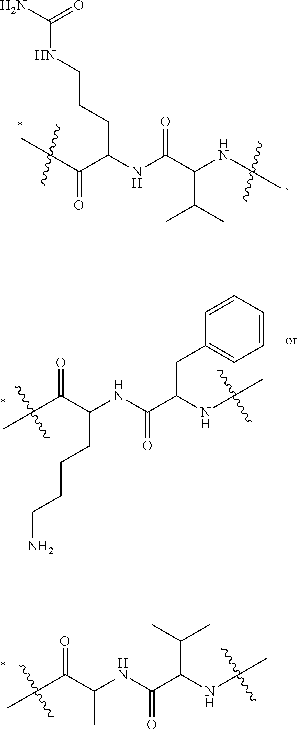 [SMILES: *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C]

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and

each t is independently selected from 1, 2, 3, 4, 5, 6, 7, 8, 9, 10, 11, 12, 13, 14, 15, 16, 17 and 18.

Embodiment 4. The compound of Formula (I) having the structure of Formula (Ia) or Formula (Ib), and the pharmaceutically acceptable salts thereof:

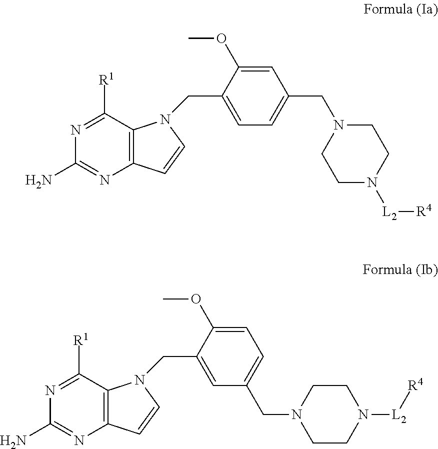

wherein:
R¹ is —NHR² or —NHCHR²R³;
R² is —C₃-C₆alkyl or —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴;
R⁴ is

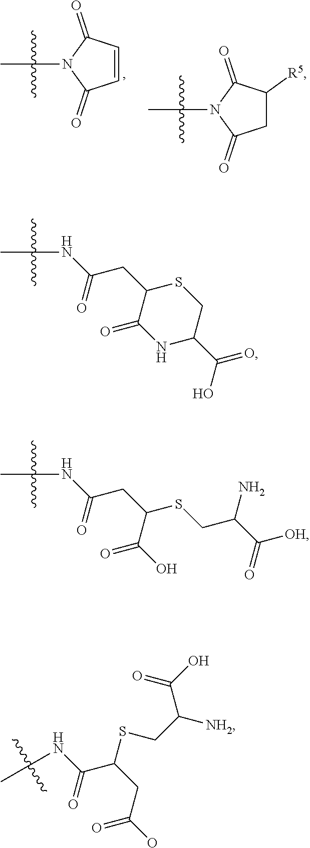

—ONH₂, —NH₂,

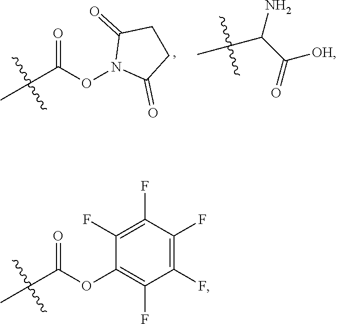 [SMILES: *C(=O)ON1C(=O)CCC1=O ; *C(N)C(=O)O ; *C(=O)Oc1c(F)c(F)c(F)c(F)c1F]

—NHC(═O)CH═CH₂, —N₃,

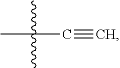 [SMILES: *C#C]

SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

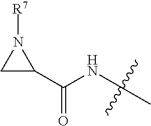

### or —CO₂H;

R⁵ is

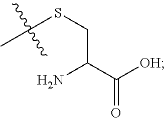 [SMILES: *SCC(N)C(=O)O]

X₁ is

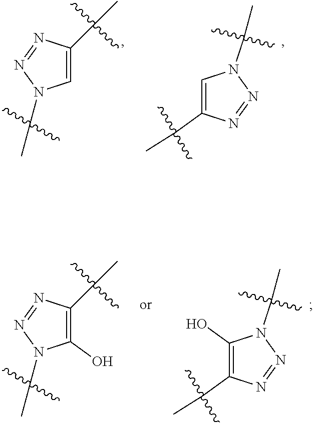 [SMILES: *c1cn(*)nn1 ; *c1cn(*)nn1 ; *c1nnn(*)c1O ; *c1nnn(*)c1O]

X₂ is

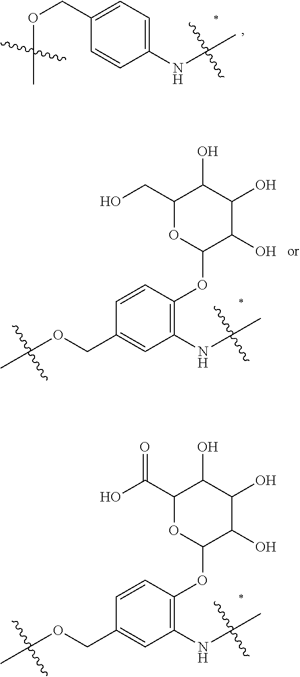 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃;
X₃ is

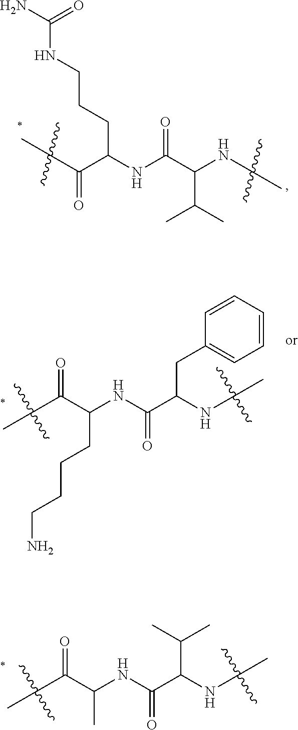 [SMILES: *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C]

where the * of X₃ indicates the point of attachment to X₂;
R⁶ is 2-pyridyl or 4-pyridyl;
each R⁷ is independently selected from H and C₁-C₆alkyl;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

Embodiment 5. The compound of Formula (I), Formula (Ia) or Formula (Ib), wherein:
R¹ is —NHR² or —NHCHR²R³;
R² is —C₄-C₆alkyl;
R³ is L₁OH;
L₁ is —(CH₂)ₘ—;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ**-, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the ** of L₂ indicates the point of attachment to R⁴;
R⁴ is

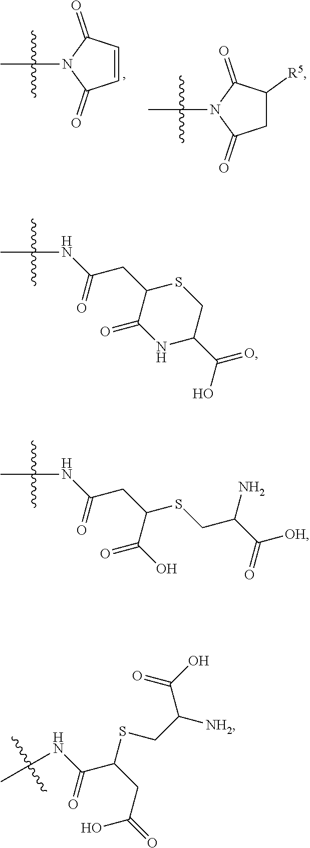

—ONH₂, —NH₂,

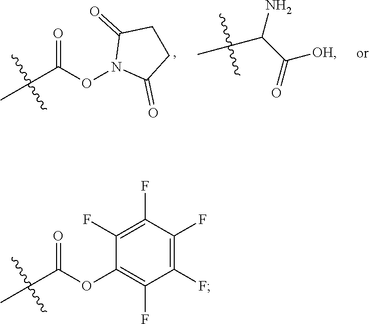 [SMILES: *C(=O)ON1C(=O)CCC1=O ; *C(N)C(=O)O ; *C(=O)Oc1c(F)c(F)c(F)c(F)c1F]

R⁵ is

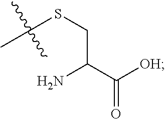 [SMILES: *SCC(N)C(=O)O]

X₁ is

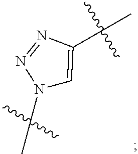 [SMILES: *c1cn(*)nn1]

X₂ is

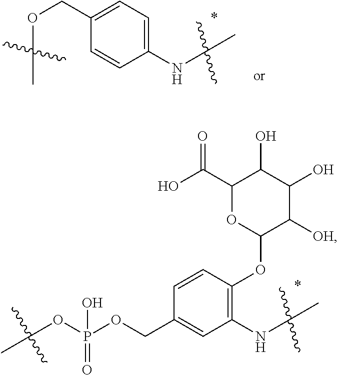 [SMILES: *Nc1ccc(CO*)cc1 ; *Nc1cc(COP(=O)(O)O*)ccc1OC1OC(C(=O)O)C(O)C(O)C1O]

where the * of X₂ indicates the point of attachment to X₃;
X₃ is

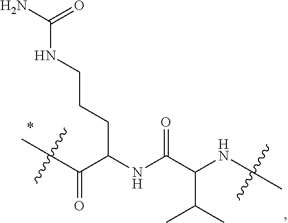 [SMILES: *NC(C(=O)NC(CCCNC(N)=O)C(*)=O)C(C)C]

where the * of X₃ indicates the point of attachment to X₂;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

Embodiment 6. The compound of Formula (I), Formula (Ia) or Formula (Ib), wherein:
R¹ is —NHR²; R² is —C₄-C₆alkyl;
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O(CH₂)ₙC(═O)NH(CH₂)ₙ—**, —((CH₂)ₙO)ₜ(CH₂)ₙNHC(═O)(CH₂)ₙ—**, —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—**, or —C(═O)(CH₂)ₙC(═O)NH(CH₂)ₙ—**, where the ** of L₂ indicates the point of attachment to R⁴;
R⁴ is

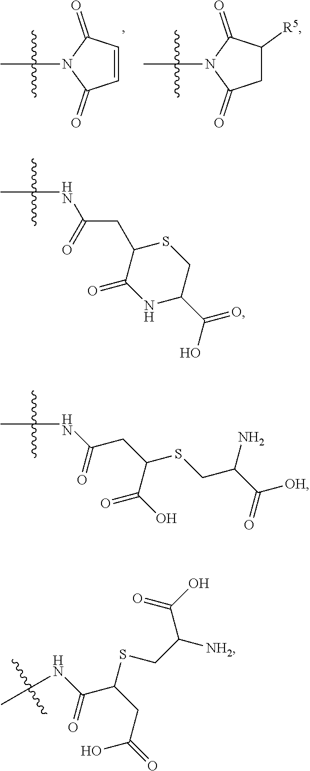

—ONH₂, —NH₂,

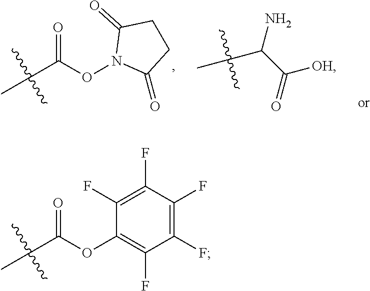 [SMILES: *C(=O)ON1C(=O)CCC1=O ; *C(N)C(=O)O ; *C(=O)Oc1c(F)c(F)c(F)c(F)c1F]

R⁵ is

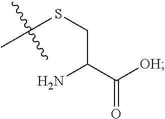 [SMILES: *SCC(N)C(=O)O]

X₁ is

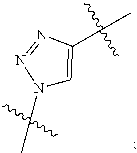 [SMILES: *c1cn(*)nn1]

X₂ is

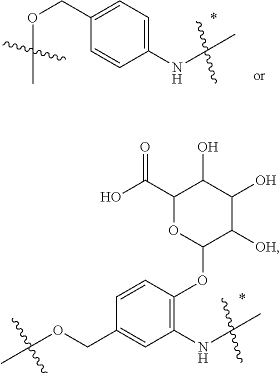 [SMILES: *Nc1ccc(CO*)cc1 ; *Nc1cc(CO*)ccc1OC1OC(C(=O)O)C(O)C(O)C1O]

where the * of X₂ indicates the point of attachment to X₃;
X₃ is

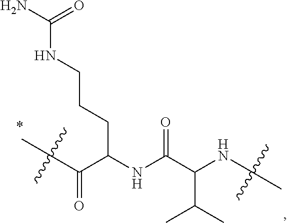 [SMILES: *NC(C(=O)NC(CCCNC(N)=O)C(*)=O)C(C)C]

where the * of X₃ indicates the point of attachment to X₂;
each n is independently selected from 1, 2, 3, and 4, and
each t is independently selected from 1, 2, 3, 4, 5, 6, 7, 8, 9, 10, 11, 12, 13, 14, 15, 16, 17 and 18.

Embodiment 7. The compound of Formula (I), Formula (Ia) or Formula (Ib), wherein:
R¹ is —NHR²;
R² is —C₄-C₆alkyl;
L₂ is —(CH₂)ₙ—, —((CH₂)ₙO)ₜ(CH₂)ₙ—**, —(CH₂)ₙX₁(CH₂)ₙ—**, —C(═O)(CH₂)ₙ—**, —C(═O)((CH₂)ₙO)ₜ(CH₂)ₙ—**, —C(═O)((CH₂)ₙO)ₜ(CH₂)ₙX₁(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—** or —C(═O)X₂C(═O)(CH₂)ₙNHC(═O)(CH₂)ₙ—**, where the ** of L₂ indicates the point of attachment to R⁴;
R⁴ is

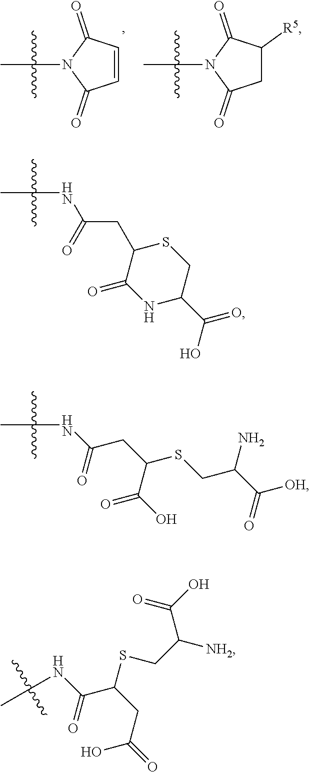

—ONH₂, —NH₂,

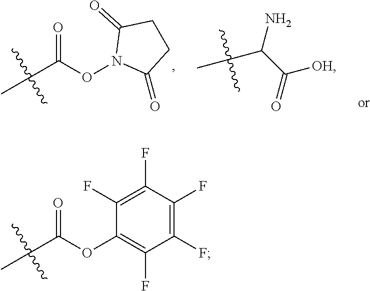 [SMILES: *C(=O)ON1C(=O)CCC1=O ; *C(N)C(=O)O ; *C(=O)Oc1c(F)c(F)c(F)c(F)c1F]

R⁵ is

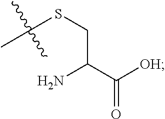 [SMILES: *SCC(N)C(=O)O]

X₁ is

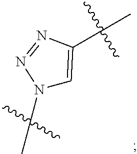 [SMILES: *c1cn(*)nn1]

X₂ is

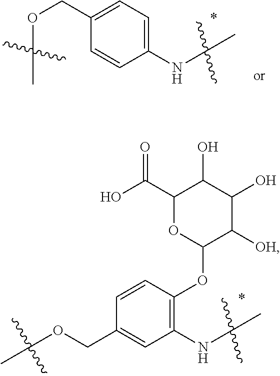 [SMILES: *Nc1ccc(CO*)cc1 ; *Nc1cc(CO*)ccc1OC1OC(C(=O)O)C(O)C(O)C1O]

where the * of X₂ indicates the point of attachment to X₃;
X₃ is

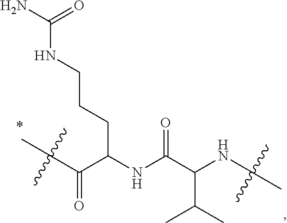 [SMILES: *NC(C(=O)NC(CCCNC(N)=O)C(*)=O)C(C)C]

where the * of X₃ indicates the point of attachment to X₂;

each n is independently selected from 1, 2, 3, and 4, and
each t is independently selected from 1, 2, 3, 4, 5, 6, 7, 8, 9, 10, 11, 12, 13, 14, 15, 16, 17 and 18.

Embodiment 8. The compound of Formula (I), Formula (Ia) or Formula (Ib), wherein:
R¹ is —NHR²;
R² is —C₄-C₆alkyl;
L₂ is —(CH₂)ₙ— or —C(═O)(CH₂)ₙ—**, where the ** of L₂ indicates the point of attachment to R⁴;
R⁴ is

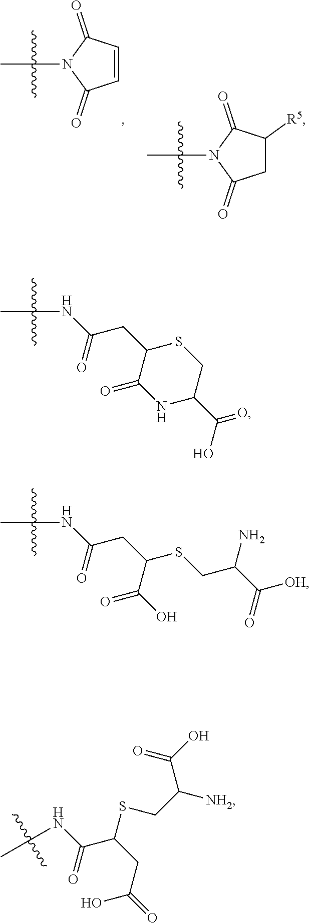

—ONH₂, —NH₂,

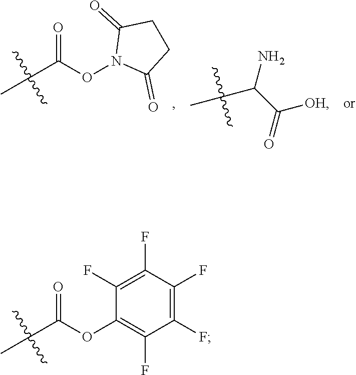 [SMILES: *C(=O)ON1C(=O)CCC1=O ; *C(N)C(=O)O ; *C(=O)Oc1c(F)c(F)c(F)c(F)c1F]

R⁵ is

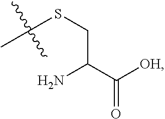 [SMILES: *SCC(N)C(=O)O]

and
each n is independently selected from 1, 2, 3, and 4.

Embodiment 9. The compound of Formula (I), Formula (Ia) or Formula (Ib), wherein:
R¹ is —NHR²;
R² is —C₄-C₆alkyl;
L₂ is —(CH₂)ₙ— or —C(═O)(CH₂)ₙ—**, where the ** of L₂ indicates the point of attachment to R⁴;
R⁴ is

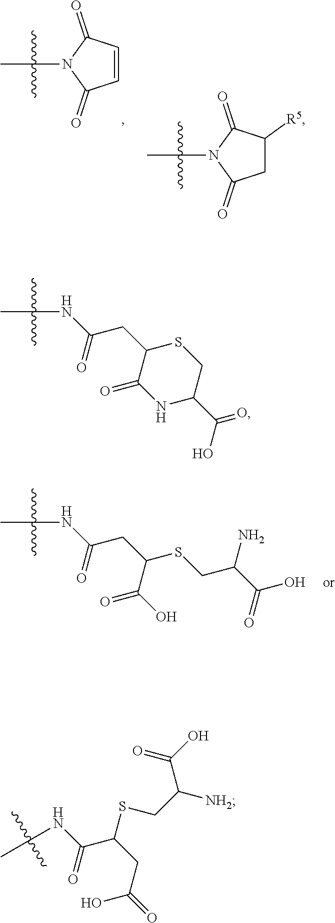

R⁵ is

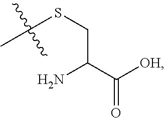 [SMILES: *SCC(N)C(=O)O]

and
each n is independently selected from 1, 2, 3, and 4.

Embodiment 10. The compound of Formula (I), Formula (Ia) or Formula (Ib), wherein:
R¹ is —NHR²;
R² is —C₄-C₆alkyl;
L₂ is —(CH₂)ₙ— or —C(═O)(CH₂)ₙ—**, where the ** of L₂ indicates the point of attachment to R⁴;
R⁴ is —ONH₂ or —NH₂;
and
each n is independently selected from 1, 2, 3, and 4.

Embodiment 11. The compound of Formula (I), Formula (Ia) or Formula (Ib), wherein:
R¹ is —NHR²;
R² is —C₄-C₆alkyl;
L₂ is —(CH₂)ₙ— or —C(═O)(CH₂)ₙ—**, where the ** of L₂ indicates the point of attachment to R⁴;
R⁴ is

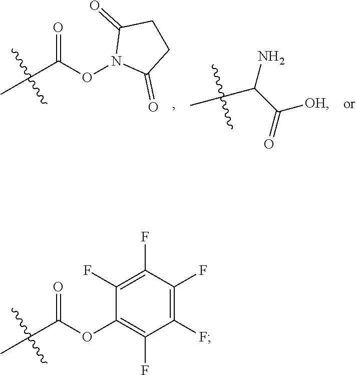 [SMILES: *C(=O)ON1C(=O)CCC1=O ; *C(N)C(=O)O ; *C(=O)Oc1c(F)c(F)c(F)c(F)c1F]

and
   each n is independently selected from 1, 2, 3, and 4.

Embodiment 12. The compound of Formula (I), Formula (Ia) or Formula (Ib), wherein: R¹ is —NHR².

Embodiment 13. The compound of Formula (I), Formula (Ia) or Formula (Ib), wherein: R¹ is —NHCHR²R³.

Embodiment 14. The compound of Formula (I), Formula (Ia) or Formula (Ib), wherein: R² is —C₄alkyl.

Embodiment 15. The compound of Formula (I), Formula (Ia) or Formula (Ib), wherein: R² is —C₅alkyl.

Embodiment 16. The compound of Formula (I), Formula (Ia) or Formula (Ib), wherein: R² is —C₆alkyl.

Embodiment 17. The compound of Formula (I), Formula (Ia) or Formula (Ib), wherein: R³ is L₁OH.

Embodiment 18. The compound of Formula (I), Formula (Ia) or Formula (Ib), wherein: L₁ is —(CH₂)—.

Embodiment 19. The compound of Formula (I), Formula (Ia) or Formula (Ib), wherein: L₁ is —(CH₂CH₂)—.

Embodiment 20. The compound of Formula (I), Formula (Ia) or Formula (Ib), wherein:
L₂ is —(CH₂)ₙ—, —((CH₂)ₙO)ₜ(CH₂)ₙ—**, —(CH₂)ₙX₁(CH₂)ₙ—**, —(CH₂)ₙNHC(═O)(CH₂)ₙ—**, —(CH₂)ₙNHC(═O(CH₂)ₙC(═O)NH(CH₂)ₙ—** or —((CH₂)ₙO)ₜ(CH₂)ₙNHC(═O)(CH₂)ₙ—**, where the * denotes attachment point to R⁴.

Embodiment 21. The compound of Formula (I), Formula (Ia) or Formula (Ib), wherein:
L₂ is —C(═O)(CH₂)ₙ—, —C(═O)((CH₂)ₙO)ₜ(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)X₂X₃C(═O)((CH₂)ₙO)ₜ(CH₂)ₙ—**, —C(═O)X₂C(═O)(CH₂)ₙNHC(═O)(CH₂)ₙ—** or —C(═O)(CH₂)ₙC(═O)NH(CH₂)ₙ—**, where the * denotes attachment point to R⁴.

Embodiment 22. The compound of Formula (I), Formula (Ia) or Formula (Ib), wherein:
L₂ is —(CH₂)ₙ— or —C(═O)(CH₂)ₙ—**, where the ** denotes attachment point to R⁴.

Embodiment 23. The compound of Formula (I), Formula (Ia) or Formula (Ib), wherein:
L₂ is —(CH₂CH₂)—** or —C(═O)(CH₂CH₂)—**, where the ** denotes attachment point to R⁴.

Embodiment 24. The compound of Formula (I), Formula (Ia) or Formula (Ib), wherein:
L₂ is —C(═O)X₂X₃C(═O)(CH₂)ₙ—**, —C(═O)X₂C(═O)(CH₂)ₙNHC(═O)((CH₂)ₙO)ₜ(CH₂)ₙ—** —C(═O)(CH₂)ₙC(R₇)₂—**, —C(═O)(CH₂)ₙC(R₇)₂SS(CH₂)ₙNHC(═O)(CH₂)ₙ—** or —(CH₂)ₙX₂C(═O)(CH₂)ₙNHC(═O)((CH₂)ₙO)ₜ(CH₂)ₙ—**, where the * denotes attachment point to R⁴.

Embodiment 25. The compound of Formula (I), Formula (Ia) or Formula (Ib), wherein:
R⁴ is

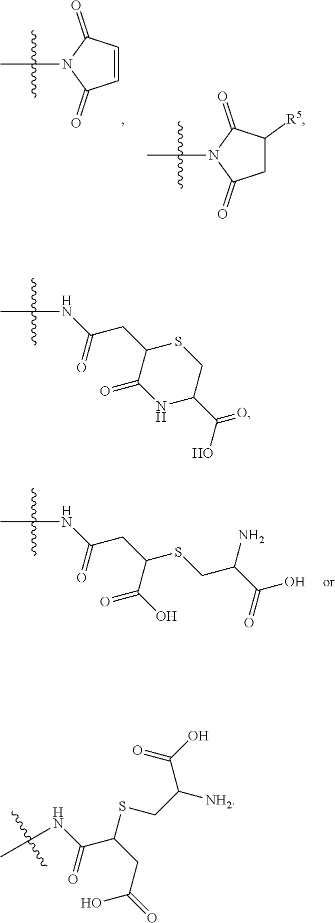

Embodiment 26. The compound of Formula (I), Formula (Ia) or Formula (Ib), wherein:
R⁴ is —ONH₂,

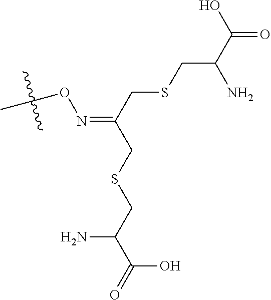 [SMILES: *ON=C(CSCC(N)C(=O)O)CSCC(N)C(=O)O]

### or —NH₂.

Embodiment 27. The compound of Formula (I), Formula (Ia) or Formula (Ib), wherein:
R⁴ is

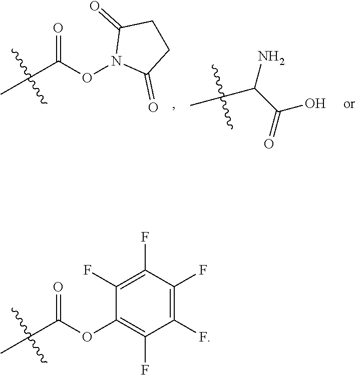 [SMILES: *C(=O)ON1C(=O)CCC1=O ; *C(N)C(=O)O ; *C(=O)Oc1c(F)c(F)c(F)c(F)c1F]

Embodiment 28. The compound of Formula (I), Formula (Ia) or Formula (Ib), wherein:
R⁴ is —NHC(═O)CH═CH₂, —N₃,

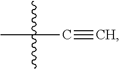 [SMILES: *C#C]

SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

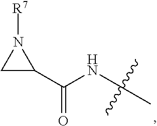

### —CO₂H, —NHCH(═O) or —NHCH(═S).

Embodiment 29. The compound of Formula (I), Formula (Ia) or Formula (Ib), wherein:
R⁴ is

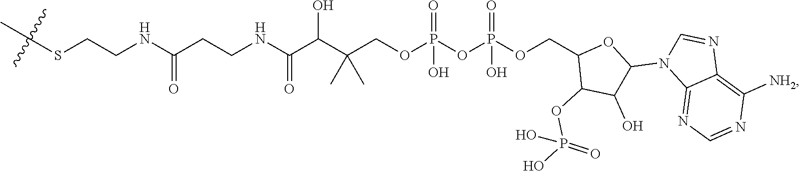 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O]

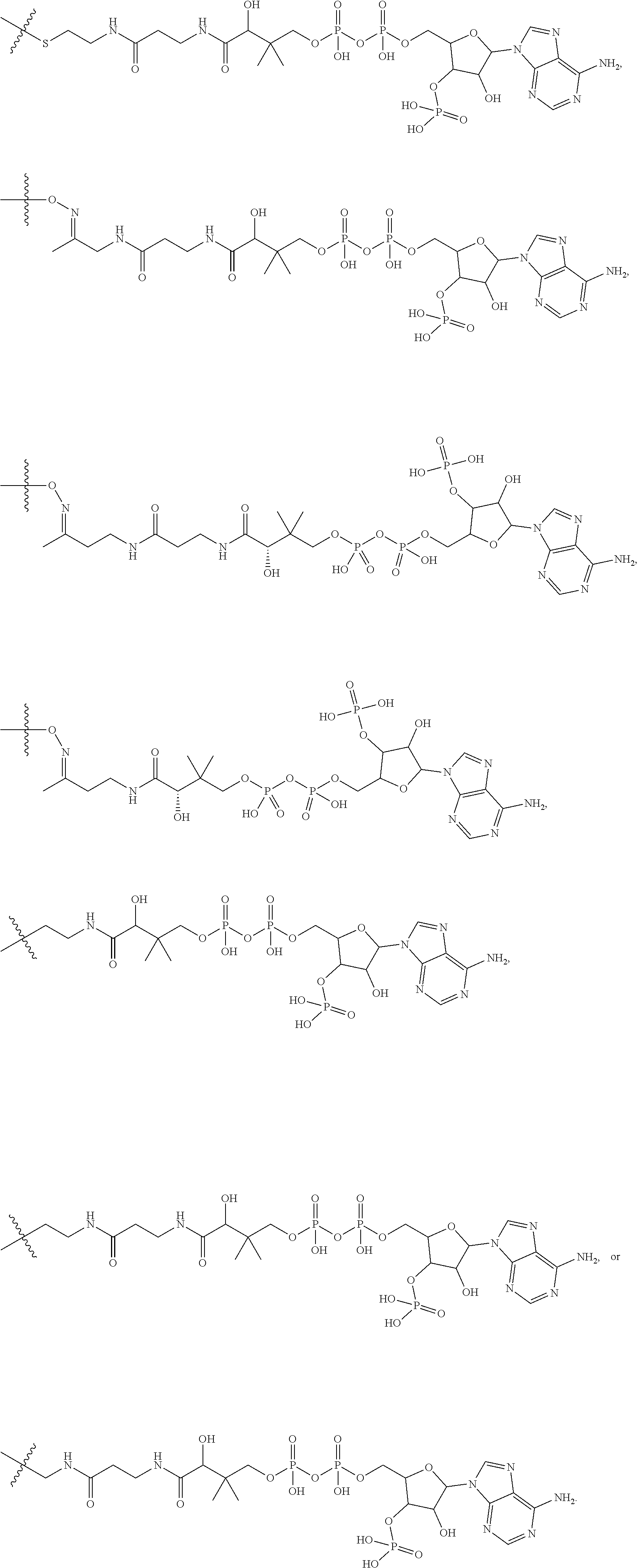

Embodiment 30. The compound of Formula (I), Formula (Ia) or Formula (Ib), wherein:

R⁴ is —SR⁷ or —OH.

Embodiment 31. The compound of Formula (I), Formula (Ia) or Formula (Ib), wherein R⁵ is

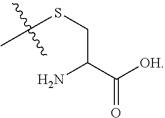 [SMILES: *SCC(N)C(=O)O]

Embodiment 32. The compound of Formula (I), Formula (Ia) or Formula (Ib), wherein: X₁ is

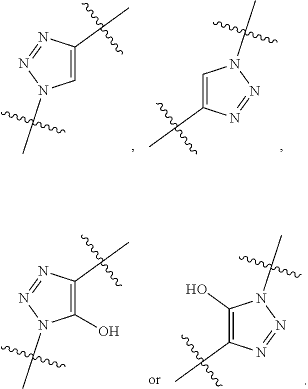 [SMILES: *c1cn(*)nn1 ; *c1cn(*)nn1 ; *c1nnn(*)c1O ; *c1nnn(*)c1O]

Embodiment 33. The compound of Formula (I), Formula (Ia) or Formula (Ib), wherein: X₁ is

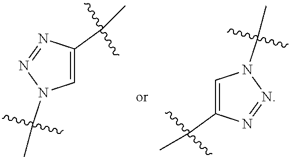 [SMILES: *c1cn(*)nn1 ; *c1cn(*)nn1]

Embodiment 34. The compound of Formula (I), Formula (Ia) or Formula (Ib), wherein: X₁ is

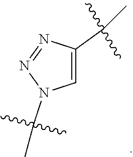 [SMILES: *c1cn(*)nn1]

Embodiment 35. The compound of Formula (I), Formula (Ia) or Formula (Ib), wherein: X₂ is

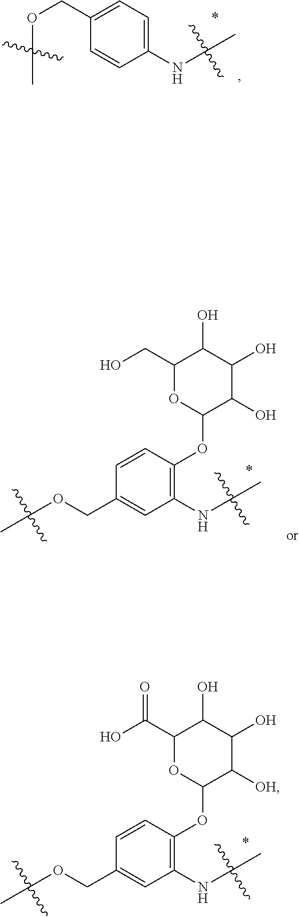 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃.

Embodiment 36. The compound of Formula (I), Formula (Ia) or Formula (Ib), wherein: X₂ is

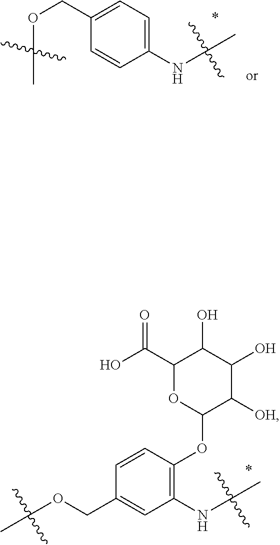 [SMILES: *Nc1ccc(CO*)cc1 ; *Nc1cc(CO*)ccc1OC1OC(C(=O)O)C(O)C(O)C1O]

where the * of X₂ indicates the point of attachment to X₃.

Embodiment 37. The compound of Formula (I), Formula (Ia) or Formula (Ib), wherein: X₂

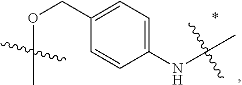 [SMILES: *Nc1ccc(CO*)cc1]

where the * of X₂ indicates the point of attachment to X₃.

Embodiment 38. The compound of Formula (I), Formula (Ia) or Formula (Ib), wherein: X₂ is

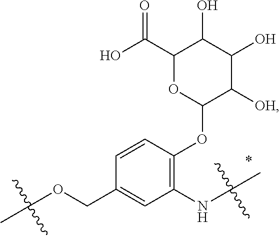 [SMILES: *Nc1cc(CO*)ccc1OC1OC(C(=O)O)C(O)C(O)C1O]

where the * of X₂ indicates the point of attachment to X₃.

Embodiment 39. The compound of Formula (I), Formula (Ia) or Formula (Ib), wherein: X₂ is

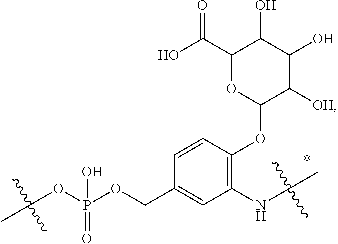 [SMILES: *Nc1cc(COP(=O)(O)O*)ccc1OC1OC(C(=O)O)C(O)C(O)C1O]

where the * of X₂ indicates the point of attachment to X₃.

Embodiment 40. The compound of Formula (I), Formula (Ia) or Formula (Ib), wherein: X₃ is

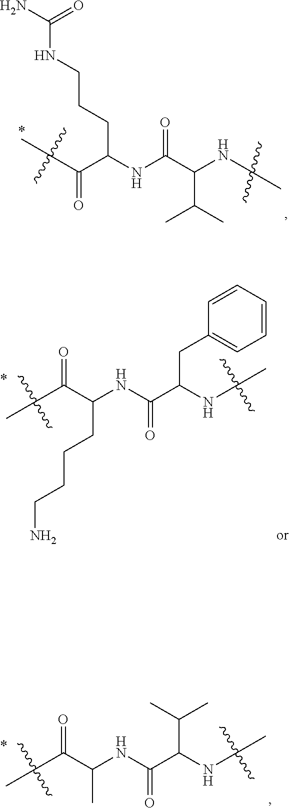 [SMILES: *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C]

where the * of X₃ indicates the point of attachment to X₂.

Embodiment 41. The compound of Formula (I), Formula (Ia) or Formula (Ib), wherein: X₃ is

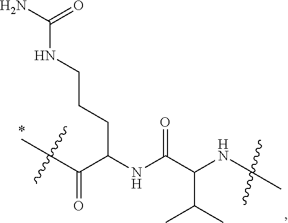 [SMILES: *NC(C(=O)NC(CCCNC(N)=O)C(*)=O)C(C)C]

where the * of X₃ indicates the point of attachment to X₂.

Embodiment 42. The compound of Formula (I), Formula (Ia) or Formula (Ib), wherein: X₃ is

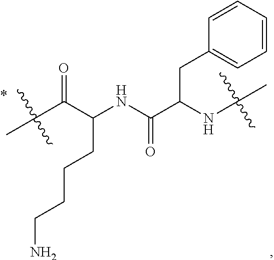 [SMILES: *NC(Cc1ccccc1)C(=O)NC(CCCCN)C(*)=O]

where the * of X₃ indicates the point of attachment to X₂.

Embodiment 43. The compound of Formula (I), Formula (Ia) or Formula (Ib), wherein: X₃ is

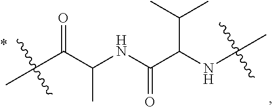 [SMILES: *NC(C(=O)NC(C)C(*)=O)C(C)C]

where the * of X₃ indicates the point of attachment to X₂.

Embodiment 44. The compound of Formula (I), Formula (Ia) or Formula (Ib), wherein: R⁶ is 2-pyridyl or 4-pyridyl.

Embodiment 45. The compound of Formula (I), Formula (Ia) or Formula (Ib), wherein: each R⁷ is independently selected from H and C₁-C₆alkyl.

Embodiment 46. The compound of Formula (I), Formula (Ia) or Formula (Ib), wherein: each R⁷ is H.

Embodiment 47. The compound of Formula (I), Formula (Ia) or Formula (Ib), wherein: each R⁷ is C₁-C₆alkyl.

Embodiment 48. The compound of Formula (I), Formula (Ia) or Formula (Ib), wherein: each m is independently selected from 1, 2, 3, and 4.

Embodiment 49. The compound of Formula (I), Formula (Ia) or Formula (Ib), wherein: each m is 1 or 2.

Embodiment 50. The compound of Formula (I), Formula (Ia) or Formula (Ib), wherein: each n is independently selected from 1, 2, 3, and 4.

Embodiment 51. The compound of Formula (I), Formula (Ia) or Formula (Ib), wherein: each n is 2 or 3.

Embodiment 52. The compound of Formula (I), Formula (Ia) or Formula (Ib), wherein: each t is independently selected from 1, 2, 3, 4, 5, 6, 7, 8, 9, 10, 11, 12, 13, 14, 15, 16, 17 and 18.

Embodiment 53. The compound of Formula (I), Formula (Ia) or Formula (Ib), wherein: each t is independently selected from 1, 2, 3, 4, 5 and 6.

Embodiment 54. The compound of Formula (I), Formula (Ia) or Formula (Ib) selected from:
1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-1H-pyrrole-2,5-dione;
(2R)-2-amino-3-((1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-2,5-dioxopyrrolidin-3-yl)thio)propanoic acid;
(6R)-6-(2-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-2-oxoethyl)-5-oxothiomorpholine-3-carboxylic acid;
3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid;
(S)-3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid;
(R)-3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid;
2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid;
(R)-2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid;
(S)-2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid;
1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-pyrrole-2,5-dione;
(2S)-2-amino-3-((1-(2-(4-(4-((2-amino-4-(pentylamino)-5H-pyrrolo[3,2-d]pyrimidin-5-yl)methyl)-3-methoxybenzyl)piperazin-1-yl)ethyl)-2,5-dioxopyrrolidin-3-yl)thio)propanoic acid;
(6R)-6-(2-((2-(4-(4-((2-amino-4-(pentylamino)-5H-pyrrolo[3,2-d]pyrimidin-5-yl)methyl)-3-methoxybenzyl)piperazin-1-yl)ethyl)amino)-2-oxoethyl)-5-oxothiomorpholine-3-carboxylic acid;
3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic acid;
(S)-3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic acid;
(R)-3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic acid;
2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic acid;
(R)-2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic acid;
(S)-2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic acid;
1-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-1H-pyrrole-2,5-dione;
3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid;
(S)-3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid;
(R)-3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid;
2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid;
(R)-2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid;
(S)-2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid;
1-(2-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethoxy)ethyl)-1H-pyrrole-2,5-dione;
(2R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic acid;
(2R,5S)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic acid;
(2R,5R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic acid;
(19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic acid;
(16R,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic acid;
(16S,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic acid;
1-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-1H-pyrrole-2,5-dione;
(2R)-2-amino-28-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,28-dioxo-10,13,16,19,22,25-hexaoxa-4-thia-7-azaoctacosan-1-oic acid;
(2R,5S)-2-amino-28-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,28-dioxo-10,13,16,19,22,25-hexaoxa-4-thia-7-azaoctacosan-1-oic acid;
(2R,5R)-2-amino-28-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,28-dioxo-10,13,16,19,22,25-hexaoxa-4-thia-7-azaoctacosan-1-oic acid;
(28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic acid;
(25R,28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic acid;
(25S,28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic acid;
1-((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)-1H-pyrrole-2,5-dione;
(2R)-2-amino-3-((2-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-2-oxoethyl)thio)pentanedioic acid;
N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-3-(2,5-dioxo-2,5-dihydro-1H-pyrrol-1-yl)propanamide;
(19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-(carboxymethyl)-1,11,15-trioxo-4,7-dioxa-17-thia-10,14-diazaicosan-20-oic acid;
(16S,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-(carboxymethyl)-1,11,15-trioxo-4,7-dioxa-17-thia-10,14-diazaicosan-20-oic acid;
(16R,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-(carboxymethyl)-1,11,15-trioxo-4,7-dioxa-17-thia-10,14-diazaicosan-20-oic acid;
(20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic acid;
(17R,20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic acid;
(17S,20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic acid;
5-(4-((4-(3-aminopropyl)piperazin-1-yl)methyl)-2-methoxybenzyl)-N4-pentyl-5H-pyrrolo[3,2-d]pyrimidine-2,4-diamine;
1-(4-(4-((2-amino-4-(pentylamino)-5H-pyrrolo[3,2-d]pyrimidin-5-yl)methyl)-3-methoxybenzyl)piperazin-1-yl)-3-(2-(2-aminoethoxy)ethoxy)propan-1-one;
N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(2,5-dioxo-2,5-dihydro-1H-pyrrol-1-yl)acetamide;
(2R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic acid;
(2R,5S)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic acid;
(2R,5R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic acid;
(19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic acid;
(16R,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic acid;
(16S,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic acid;
4-(4-((2-amino-4-(pentylamino)-5H-pyrrolo[3,2-d]pyrimidin-5-yl)methyl)-3-methoxybenzyl)-N-(2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl)piperazine-1-carboxamide;
3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid;
(S)-3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid;
(R)-3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid;
2-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid;
(R)-2-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid;
(S)-2-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid;
1-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)-1H-pyrrole-2,5-dione;
3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid;
(S)-3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid;
(R)-3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid;
2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid;
(R)-2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid;
(S)-2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid;
1-((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)-1H-pyrrole-2,5-dione;
3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic acid;
(S)-3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic acid;
(R)-3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic acid;
2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic acid;
(R)-2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic acid;
(S)-2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic acid;
N-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-3-(2,5-dioxo-2,5-dihydro-1H-pyrrol-1-yl)propanamide;
4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate;
(2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2,5-dioxo-2,5-dihydro-1H-pyrrol-1-yl)propanamido)propanamido)phenoxy)-3,4,5-trihydroxytetrahydro-2H-pyran-2-carboxylic acid;
(S)-1-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropyl)-1H-pyrrole-2,5-dione;
1-(3-(4-(3-((2-amino-4-(pentylamino)-5H-pyrrolo[3,2-d]pyrimidin-5-yl)methyl)-4-methoxybenzyl)piperazin-1-yl)-3-oxopropyl)-1H-pyrrole-2,5-dione;
3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid;
(S)-3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid;
(R)-3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid;
2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid;
(R)-2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid;
(S)-2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid;
1-(4-(4-((2-amino-4-(pentylamino)-5H-pyrrolo[3,2-d]pyrimidin-5-yl)methyl)-3-methoxybenzyl)piperazin-1-yl)-2-(aminooxy)ethanone;
1-(4-(4-((2-amino-4-(pentylamino)-5H-pyrrolo[3,2-d]pyrimidin-5-yl)methyl)-3-methoxybenzyl)piperazin-1-yl)-3-(2-aminoethoxy)propan-1-one;
N-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-2-(aminooxy)acetamide;
(S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-2-(aminooxy)ethanone;
(S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-(2-(2-aminoethoxy)ethoxy)propan-1-one;
(S)—N-(2-(2-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropoxy)ethoxy)ethyl)-2-(aminooxy)acetamide;
N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(aminooxy)acetamide;
5-(4-((4-(2-(2-(aminooxy)ethoxy)ethyl)piperazin-1-yl)methyl)-2-methoxybenzyl)-N4-pentyl-5H-pyrrolo[3,2-d]pyrimidine-2,4-diamine;
N-(3-(4-(4-((2-amino-4-(pentylamino)-5H-pyrrolo[3,2-d]pyrimidin-5-yl)methyl)-3-methoxybenzyl)piperazin-1-yl)propyl)-2-(aminooxy)acetamide;
5-(4-((4-(2-(2-(2-aminoethoxy)ethoxy)ethyl)piperazin-1-yl)methyl)-2-methoxybenzyl)-N4-pentyl-5H-pyrrolo[3,2-d]pyrimidine-2,4-diamine;
N-(2-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethoxy)ethyl)-2-(aminooxy)acetamide;
2,5-dioxopyrrolidin-1-yl 5-(4-(4-((2-amino-4-(pentylamino)-5H-pyrrolo[3,2-d]pyrimidin-5-yl)methyl)-3-methoxybenzyl) piperazin-1-yl)-5-oxopentanoate;
(S)-2,5-dioxopyrrolidin-1-yl 5-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-5-oxopentanoate;
(S)-2-amino-6-(5-(4-(3-((2-amino-4-(((S)-1-hydroxyhexan-2-yl)amino)-5H-pyrrolo[3,2-d]pyrimidin-5-yl)methyl)-4-methoxybenzyl)piperazin-1-yl)-5-oxopentanamido)hexanoic acid;
(S)-2-amino-6-(5-(4-(4-((2-amino-4-(pentylamino)-5H-pyrrolo[3,2-d]pyrimidin-5-yl)methyl)-3-methoxybenzyl)piperazin-1-yl)-5-oxopentanamido)hexanoic acid;
2,5-dioxopyrrolidin-1-yl 5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanoate;
(S)-2-amino-6-(5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanamido)hexanoic acid;
2,5-dioxopyrrolidin-1-yl 5-(4-(3-((2-amino-4-(pentylamino)-5H-pyrrolo[3,2-d]pyrimidin-5-yl)methyl)-4-methoxybenzyl) piperazin-1-yl)-5-oxopentanoate;
(S)-2-amino-6-(5-(4-(3-((2-amino-4-(pentylamino)-5H-pyrrolo[3,2-d]pyrimidin-5-yl)methyl)-4-methoxybenzyl)piperazin-1-yl)-5-oxopentanamido)hexanoic acid;
perfluorophenyl 5-(4-(4-((2-amino-4-(pentylamino)-5H-pyrrolo[3,2-d]pyrimidin-5-yl)methyl)-3-methoxybenzyl)piperazin-1-yl)-5-oxopentanoate;
perfluorophenyl 3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanoate;
perfluorophenyl 3-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)propanoate;
(S)-2-amino-6-(3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanamido)hexanoic acid, and
N-(15-(4-(4-((2-amino-4-(pentylamino)-5H-pyrrolo[3,2-d]pyrimidin-5-yl)methyl)-3-methoxybenzyl)piperazin-1-yl)-15-oxo-3,6,9,12-tetraoxapentadecyl)-5-((3aS,4S,6aR)-2-oxohexahydro-1H-thieno[3,4-d]imidazol-4-yl)pentanamide.

Embodiment 55. The compound of Formula (I), Formula (Ia) or Formula (Ib) selected from:
1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-1H-pyrrole-2,5-dione;
1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-pyrrole-2,5-dione;
1-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-1H-pyrrole-2,5-dione, and
1-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)-1H-pyrrole-2,5-dione.

Embodiment 56. The compound of Formula (I), Formula (Ia) or Formula (Ib) selected from:
(2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic acid;
4-((R)-6-amino-2-((S)-2-(3-(2,5-dioxo-2,5-dihydro-1H-pyrrol-1-yl)propanamido)-3-phenylpropanamido)hexanamido)benzyl 4-(4-((2-amino-4-(pentylamino)-5H-pyrrolo[3,2-d]pyrimidin-5-yl)methyl)-3-methoxybenzyl)piperazine-1-carboxylate;
4-((S)-2-((S)-2-(3-(2-(2,5-dioxo-2,5-dihydro-1H-pyrrol-1-yl)ethoxy)propanamido)-3-methylbutanamido)propanamido)benzyl 4-(4-((2-amino-4-(pentylamino)-5H-pyrrolo[3,2-d]pyrimidin-5-yl)methyl)-3-methoxybenzyl)piperazine-1-carboxylate;
(2S,3S,4S,5R,6S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2,5-dioxo-2,5-dihydro-1H-pyrrol-1-yl)propanamido)propanamido)phenoxy)-3,4,5-trihydroxytetrahydro-2H-pyran-2-carboxylic acid;
(2S,3S,4S,5R,6S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic acid;
N-(2-((5-(4-(4-((2-amino-4-(pentylamino)-5H-pyrrolo[3,2-d]pyrimidin-5-yl)methyl)-3-methoxybenzyl)piperazin-1-yl)-2-methyl-5-oxopentan-2-yl)disulfanyl)ethyl)-3-(2,5-dioxo-2,5-dihydro-1H-pyrrol-1-yl)propanamide;
1-(4-(4-((2-amino-4-(pentylamino)-5H-pyrrolo[3,2-d]pyrimidin-5-yl)methyl)-3-methoxybenzyl)piperazin-1-yl)-4-methyl-4-(methylthio)pentan-1-one; (2S,3S,4S,5R,6S)-6-(4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic acid;
(2R,2′R)-3,3′-((2-((2-(4-(4-((2-amino-4-(pentylamino)-5H-pyrrolo[3,2-d]pyrimidin-5-yl)methyl)-3-methoxybenzyl)piperazin-1-yl)-2-oxoethoxy)imino)propane-1,3-diyl)bis(sulfanediyl))bis(2-aminopropanoic acid);
(R)-2-amino-6-((((R)-2-amino-2-carboxyethyl)thio)methyl)-17-(4-(4-((2-amino-4-(pentylamino)-5H-pyrrolo[3,2-d]pyrimidin-5-yl)methyl)-3-methoxybenzyl)piperazin-1-yl)-10,17-dioxo-8,14-dioxa-4-thia-7,11-diazaheptadec-6-enoic acid, and
2-(4-(4-((2-amino-4-(pentylamino)-5H-pyrrolo[3,2-d]pyrimidin-5-yl)methyl)-3-methoxybenzyl)piperazin-1-yl)ethan-1-ol.

In some embodiments, the immunostimulatory molecules of the invention are RIG-1 agonists having the following structures:

a)
(SEQ ID NO: 334)
5′ ppp-GGACGUACGC (UUCG) GCGUACGUCC-3′
b)
(SEQ ID NO: 335)
5′ ppp-GGACGUACGC (UXCG) GCGUACGUCC-3′
c)
(SEQ ID NO: 336)
5′OH-GGACGUACGC (UUCG) GCGUACGUCC-3′
or
d)
(SEQ ID NO: 337)
5′OH-GGACGUACGC (UXCG) GCGUACGUCC-3′

where:
   ppp-G is

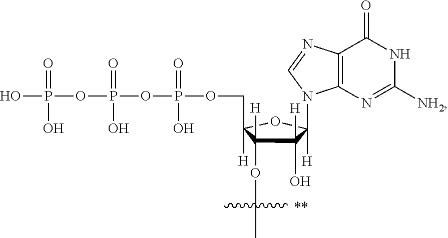

where the * of ppp-G is the point of attachment toward the 3′ end;
   OH-G is

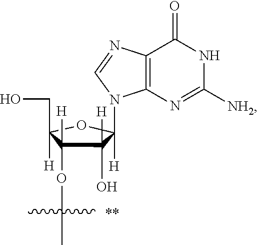

where the * of OH-G is the point of attachment toward the 3′ end;
   G is

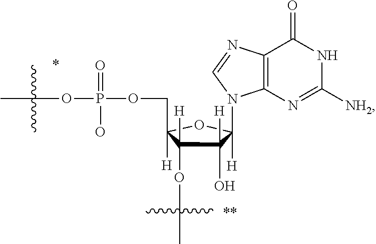

where the * of G is the point of attachment toward the 5′ end and the ** of G is the point of attachment toward the 3′ end;
   A is

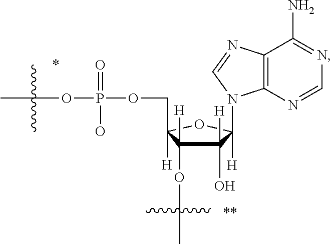

where the * of A is the point of attachment toward the 5′ end and the ** of A is the point of attachment the point of attachment toward the 3′ end;
   C is

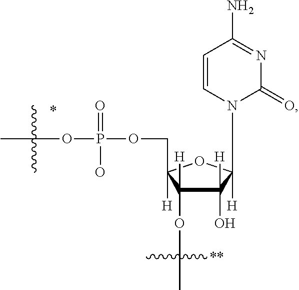

where the * of C is the point of attachment toward the 5′ end and the ** of C is the point of attachment toward the 3′ end;
   or if C is in a 3′ terminal position, then C is

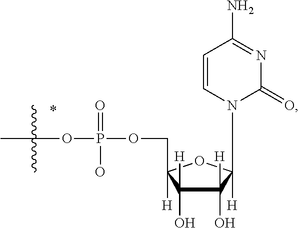

where the * of C is the point of attachment toward the 5′ end;
   U is

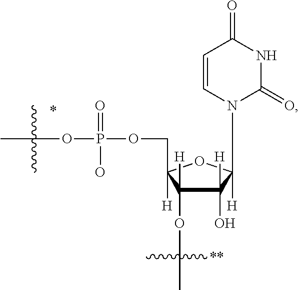

where the * of U is the point of attachment toward the 5′ end and the ** of U is the point of attachment toward the 3′ end;
   and
   X is

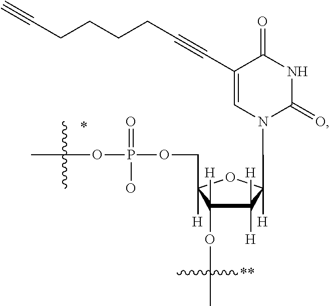

where the * of X is the point of attachment toward the 5′ end and the ** of X is the point of attachment toward the 3′ end.

In other embodiments, the immunostimulatory molecules of the invention are RIG-1 agonists having the structure of Formula (IV):

RIGIa-L-R⁴ (IV)

wherein:
RIGIa is a RIG-1 agonist selected from:

a)
(SEQ ID NO: 338)
5′ ppp-GGACGUACGC (UX_{M}CG) GCGUACGUCC-3 ′
or
b)
(SEQ ID NO: 339)
5′OH-GGACGUACGC (UX_{M}CG) GCGUACGUCC-3 ′

where:
   ppp-G is

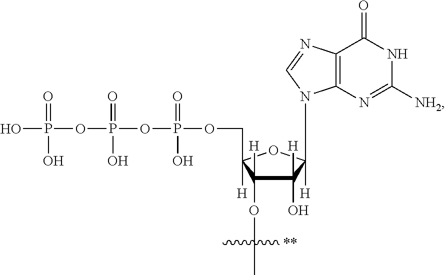

where the ** of ppp-G is the point of attachment toward the 3′ end;
   OH-G is

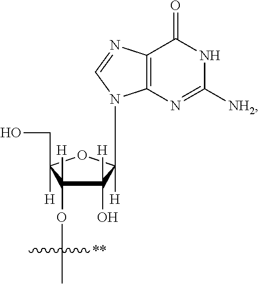

where the ** of OH-G is the point of attachment toward the 3′ end;
   G is

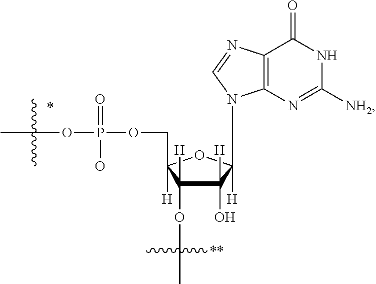

where the * of G is the point of attachment toward the 5′ end and the ** of G is the point of attachment toward the 3′ end;
   A is

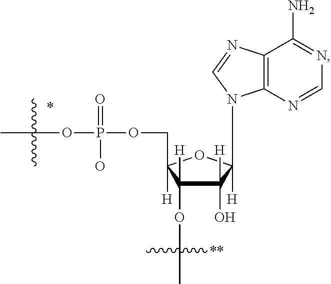

where the * of A is the point of attachment toward the 5′ end and the ** of A is the point of attachment the point of attachment toward the 3′ end;
   C is

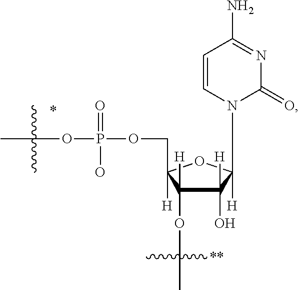

where the * of C is the point of attachment toward the 5′ end and the ** of C is the point of attachment toward the 3′
   or if C is in a 3′ terminal position, then C is

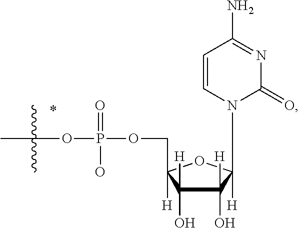

where the * of C is the point of attachment toward the 5′ end;
   U is

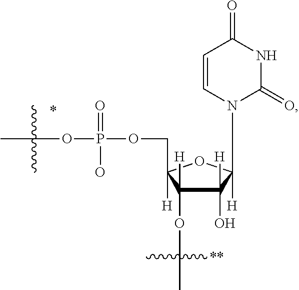

where the * of U is the point of attachment toward the 5′ end and the ** of U is the point of attachment toward the 3′ end;
   and
   X_{M} is

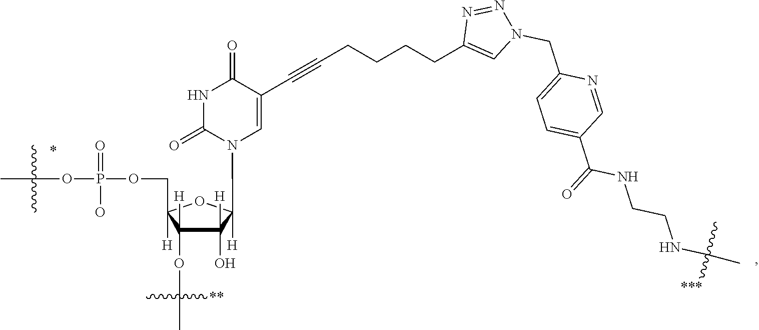

where the * of X_{M} is the point of attachment toward the 5′ end, the ** of X_{M} is the point of attachment toward the 3′ end and the *** of X_{M} is the point of attachment to L;
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—**; —C(═O)X₂X₃((CH₂)ₙO)ₜ(CH₂)ₙ—**, —C(═O)X₂X₃C(═O)(CH₂)ₘO(CH₂)ₘC(═O)—**; —(CH₂)ₙNHC(═O)(CH₂)ₙ—, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)O(CH₂)ₙC(R₇)₂SS(CH₂)ₙNHC(═O)(CH₂)ₙ—** or —C(═O)(CH₂)ₙC(═O)NH(CH₂)ₙ—**, where the ** of L indicates the point of attachment to R⁴;
R⁴ is

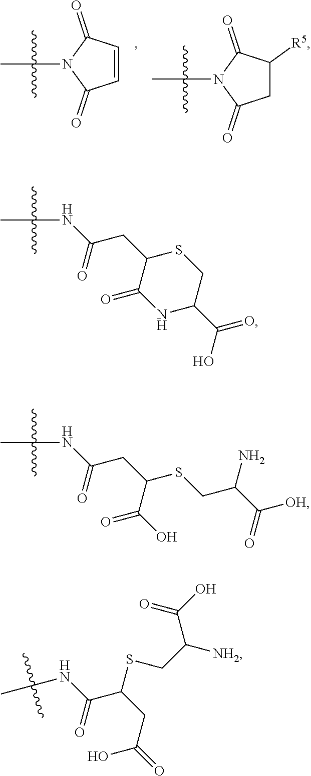

—ONH₂, —NH₂,

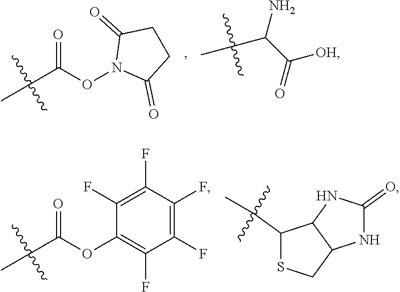

### —N₃,

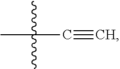 [SMILES: *C#C]

—NHC(═O)CH═CH₂, —SH, —SR⁷, —OH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

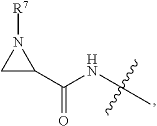

—CO₂H, —C(O)NHNH₂,

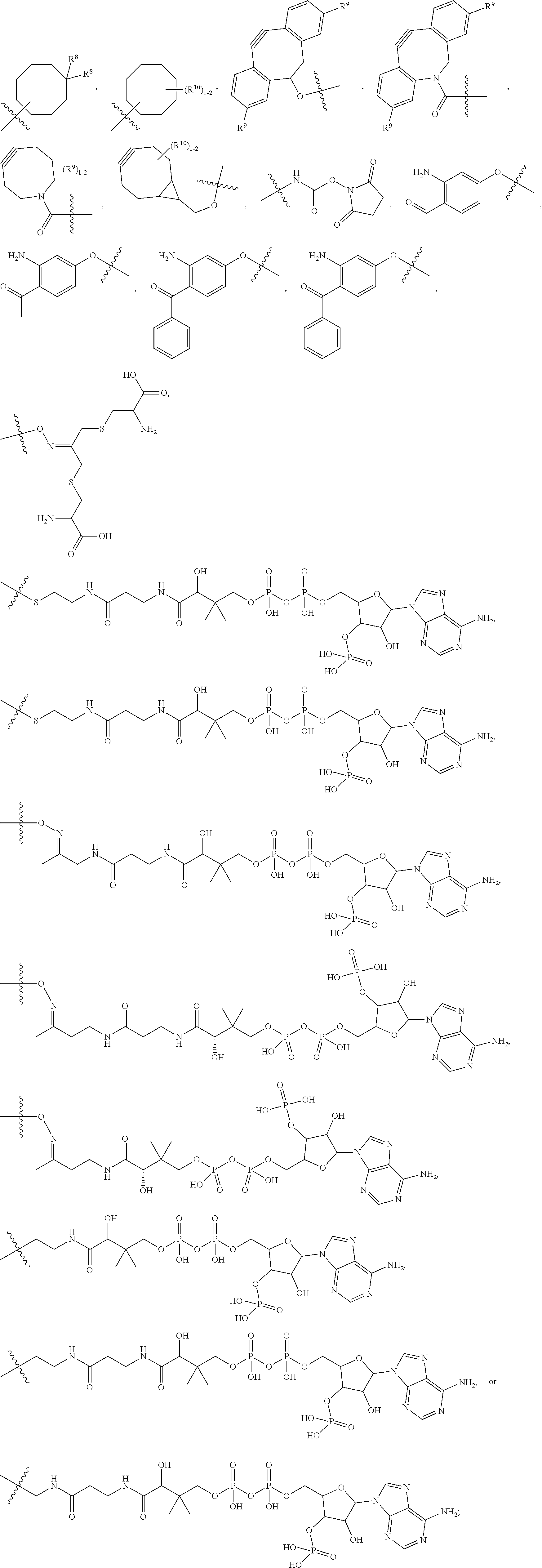

R⁵ is 0

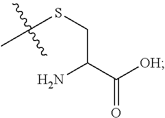 [SMILES: *SCC(N)C(=O)O]

X₁ is

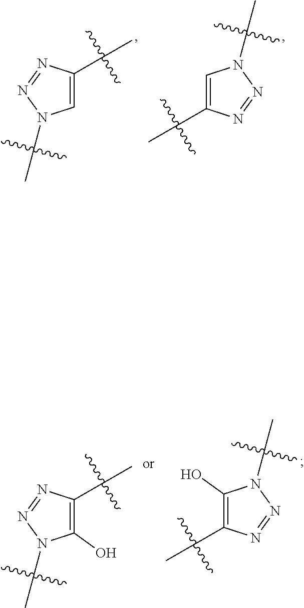 [SMILES: *c1cn(*)nn1 ; *c1cn(*)nn1 ; *c1nnn(*)c1O ; *c1nnn(*)c1O]

X₂ is

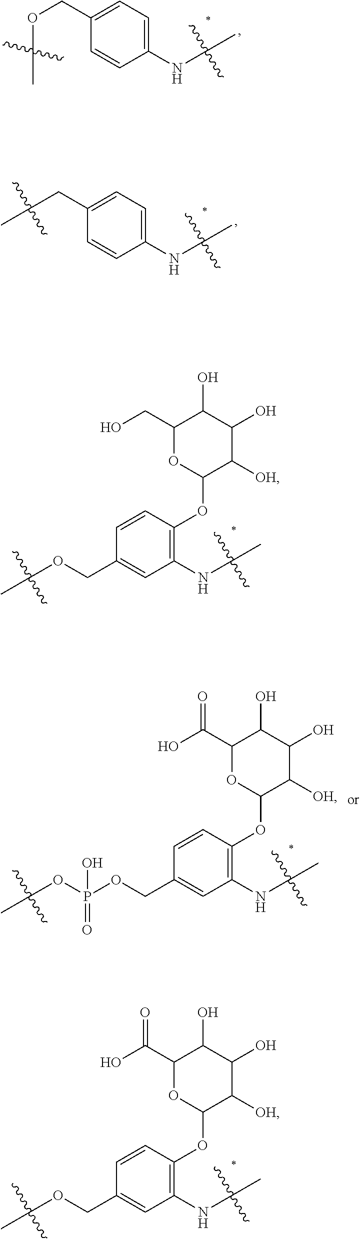 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the * of X₂ indicates the point of attachment to X₃;
X₃ is

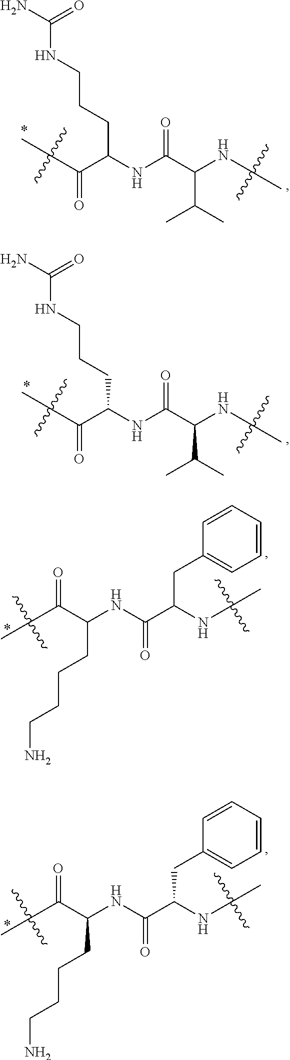

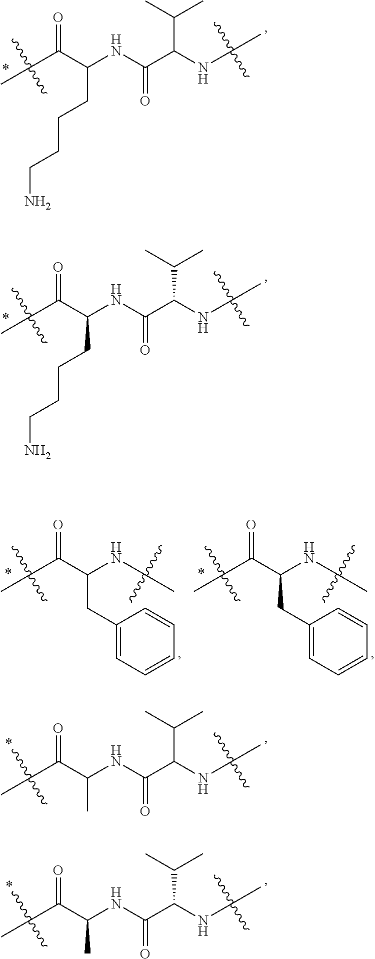

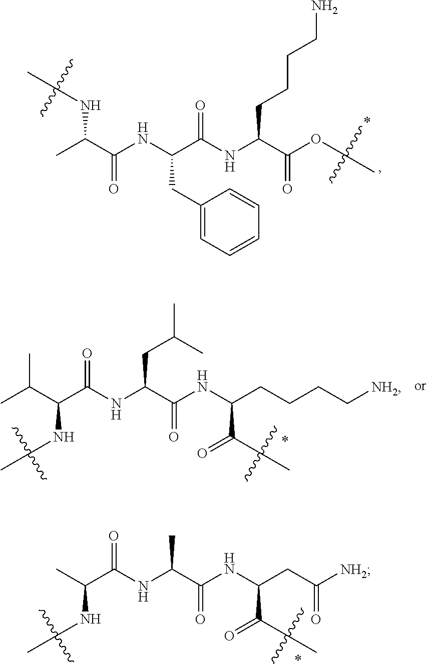 [SMILES: *N[C@@H](C)C(=O)N[C@@H](Cc1ccccc1)C(=O)N[C@@H](CCCCN)C(=O)O* ; *N[C@H](C(=O)N[C@@H](CC(C)C)C(=O)N[C@@H](CCCCN)C(*)=O)C(C)C ; *N[C@@H](C)C(=O)N[C@@H](C)C(=O)N[C@@H](CC(N)=O)C(*)=O]

where the * of X₃ indicates the point of attachment to X₂;
R⁶ is 2-pyridyl or 4-pyridyl;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, and 4;
each n is independently selected from 1, 2, 3, and 4;
and
each t is independently selected from 1, 2, 3, 4, 5, 6, 7, 8, 9, 10, 11, 12, 13, 14, 15, 16, 17 and 18.

Certain aspects and examples of the compounds of Formula (IV) are provided in the following listing of additional, enumerated embodiments. It will be recognized that features specified in each embodiment may be combined with other specified features to provide further embodiments of the present invention.

Embodiment 57. The compound of Formula (IV), wherein:
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—** or —C(═O)X₂X₃((CH₂)ₙO)ₜ(CH₂)ₙ—**, where the * denotes attachment point to R⁴.

Embodiment 58. The compound of Formula (IV), wherein:
L is —C(═O)(CH₂)ₙ—**, —C(═O)((CH₂)ₙO)ₜ(CH₂)ₙ—**, —C(═O)X₂X₃C(═O)(CH₂)ₙ—** or —C(═O)X₂X₃((CH₂)ₙO)ₜ(CH₂)ₙ—**, where the * denotes attachment point to R⁴.

Embodiment 59. The compound of Formula (IV), wherein:
L is —C(═O)(CH₂)ₙ—** or —C(═O)X₂X₃C(═O)(CH₂)ₙ—**, where the * denotes attachment point to R⁴.

Embodiment 60. The compound of Formula (IV), wherein:
L is —C(═O)(CH₂)ₙ—**, where the * denotes attachment point to R⁴.

Embodiment 61. The compound of Formula (IV), wherein:
L is —C(═O)(CH₂CH₂—**, where the * denotes attachment point to R⁴.

Embodiment 62. The compound of Formula (IV), wherein:
R⁴ is

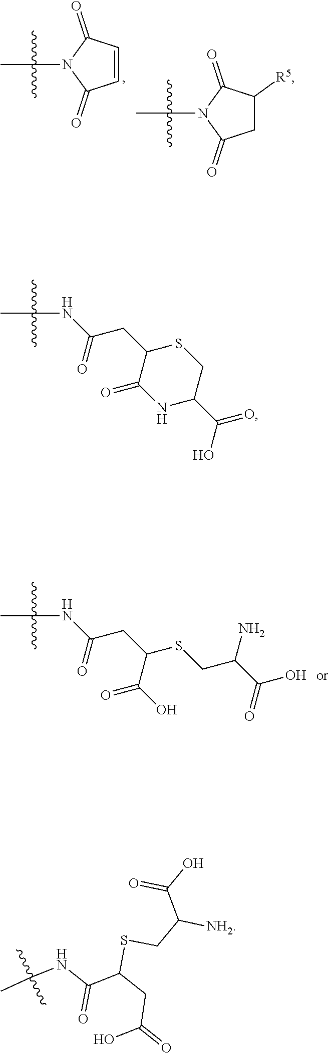

Embodiment 63. The compound of Formula (IV), wherein:
R⁴ is —ONH₂.

Embodiment 64. The compound of Formula (IV), wherein:
R⁴ is

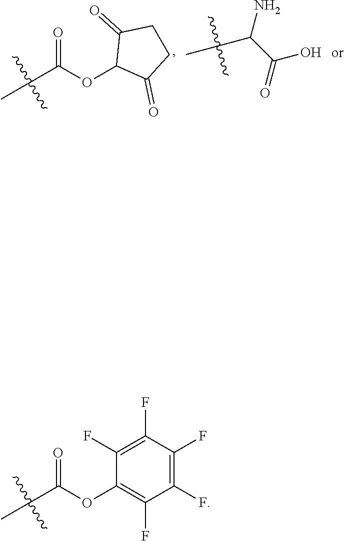 [SMILES: *C(=O)OC1C(=O)CCC1=O ; *C(N)C(=O)O ; *C(=O)Oc1c(F)c(F)c(F)c(F)c1F]

Embodiment 65. The compound of Formula (IV), wherein, X₁ is

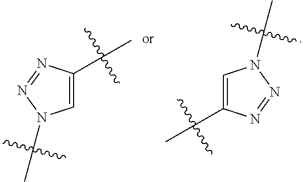 [SMILES: *c1cn(*)nn1 ; *c1cn(*)nn1]

Embodiment 66. The compound of Formula (IV), wherein, X₁ is

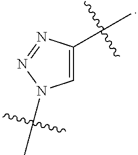 [SMILES: *c1cn(*)nn1]

Embodiment 67. The compound of Formula (IV), wherein: X₂ is

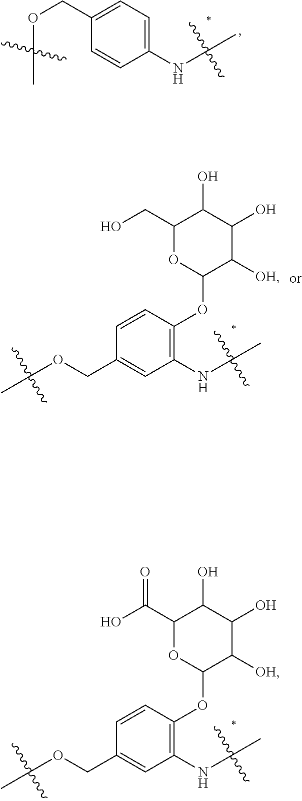 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

where the * of X₂ indicates the point of attachment to X₃.

Embodiment 68. The compound of Formula (IV), wherein: X₂ is H

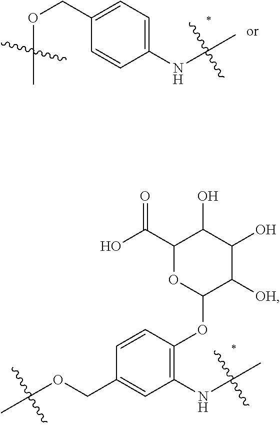 [SMILES: *Nc1ccc(CO*)cc1 ; *Nc1cc(CO*)ccc1OC1OC(C(=O)O)C(O)C(O)C1O]

where the * of X₂ indicates the point of attachment to X₃.

Embodiment 69. The compound of Formula (IV), wherein, wherein: X₂ is

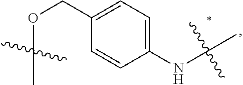 [SMILES: *Nc1ccc(CO*)cc1]

where the * of X₂ indicates the point of attachment to X₃.

Embodiment 70. The compound of Formula (IV), wherein: X₂ is

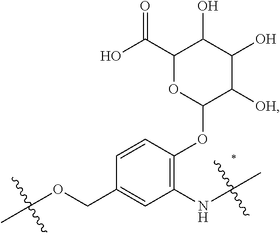 [SMILES: *Nc1cc(CO*)ccc1OC1OC(C(=O)O)C(O)C(O)C1O]

where the * of X₂ indicates the point of attachment to X₃.

Embodiment 71. The compound of Formula (IV), wherein: X₃ is

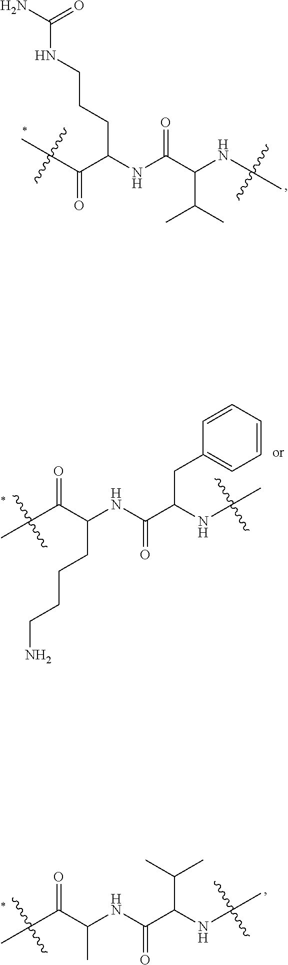 [SMILES: *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C]

where the * of X₃ indicates the point of attachment to X₂.

Embodiment 72. The compound of Formula (IV), wherein: X₃ is

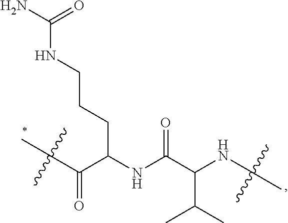 [SMILES: *NC(C(=O)NC(CCCNC(N)=O)C(*)=O)C(C)C]

where the * of X₃ indicates the point of attachment to X₂.

Embodiment 73. The compound of Formula (IV), wherein: X₃ is

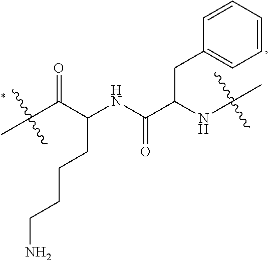 [SMILES: *NC(Cc1ccccc1)C(=O)NC(CCCCN)C(*)=O]

where the * of X₃ indicates the point of attachment to X₂.

Embodiment 74. The compound of Formula (IV), wherein: X₃ is

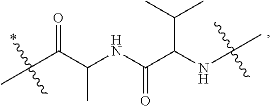

where the * of X₃ indicates the point of attachment to X₂.

Embodiment 75. The compound of Formula (IV), wherein: R⁶ is 2-pyridyl or 4-pyridyl.

Embodiment 76. The compound of Formula (IV), wherein: each R⁷ is independently selected from H and C₁-C₆alkyl.

Embodiment 77. The compound of Formula (IV), wherein: each R⁷ is H.

Embodiment 78. The compound of Formula (IV), wherein: each R⁷ is C₁-C₆alkyl.

Embodiment 79. The compound of Formula (IV), wherein: each m is independently selected from 1, 2, 3, and 4.

Embodiment 80. The compound of Formula (IV), wherein: each m is 1 or 2.

Embodiment 81. The compound of Formula (IV), wherein: each n is independently selected from 1, 2, 3, and 4.

Embodiment 82. The compound of Formula (IV), wherein: each n is 2 or 3.

Embodiment 83. The compound of Formula (IV), wherein: each t is independently selected from 1, 2, 3, 4, 5, 6, 7, 8, 9, 10, 11, 12, 13, 14, 15, 16, 17 and 18.

Embodiment 84. The compound of Formula (IV), wherein: each t is independently selected from 1, 2, 3, 4, 5 and 6.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

### Processes for Making Compounds of Formula (I) and Subformulae Thereof

General procedures for preparing compounds of Formula (I), and sub-Formulae thereof, are described herein. In the reactions described, reactive functional groups, for example hydroxy, amino, imino, thiol or carboxy groups, where these are desired in the final product, may be protected to avoid their unwanted participation in the reactions. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/1, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

In certain embodiments, compounds of Formula (I) and subformulae thereof, provided herein are prepared as a pharmaceutically acceptable acid addition salt by reacting the free base form of a compound of Formula (I) and subformulae thereof, with a stoichiometric amount of an appropriate pharmaceutically acceptable organic acid or inorganic acid or a suitable anion exchange reagent.

Such reactions are typically carried out in water or in an organic solvent, or in a mixture of the two. Generally, use of non-aqueous media like ether, ethyl acetate, ethanol, isopropanol, or acetonitrile is desirable, where practicable.

Alternatively, the salt forms of compounds of Formula (I) and subformulae thereof, are prepared using salts of the starting materials or intermediates.

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

Pharmaceutically acceptable acid addition salts of compounds of Formula (I) and subformulae thereof, include, but are not limited to, a acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlorotheophyllinate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete and xinafoate salt forms.

The organic acid or inorganic acids used to form certain pharmaceutically acceptable acid addition salts of compounds of Formula (I) and subformulae thereof, include, but are not limited to, acetic acid, adipic acid, ascorbic acid, aspartic acid, benzoic acid, benzenesulfonic acid, carbonic acid, camphor sulfonic acid, capric acid, chlorotheophyllinate, citric acid, ethanedisulfonic acid, fumaric acid, D-glycero-D-gulo-Heptonicacid, galactaric aid, galactaric acid/mucic acid, gluceptic acid, glucoheptonoic acid, gluconic acid, glucuronic acid, glutamatic acid, glutaric acid, glycolic acid, hippuric acid, hydrobromic acid, hydrochloric acid, hydroiodic acid, isethionic acid, lactic acid, lactobionic acid, lauryl sulfuric acid, malic acid, maleic acid, malonic acid, mandelic acid, mesylic acid, methanesulfonic acid, mucic acid, naphthoic acid, 1-hydroxy-2-naphthoic acid, naphthalenesulfonic acid, 2-naphthalenesulfonic acid, nicotinic acid, nitric acid, octadecanoic acid, oleaic acid, oxalic acid, palmitic acid, pamoic acid, phosphoric acid, polygalacturonic acid, propionic acid, sebacic acid, stearic acid, succinic acid, sulfosalicylic acid, sulfuric acid, tartaric acid, p-toluenesulfonic acid, trifluoroacetic acid and triphenylacetic acid.

In one embodiment, the present invention provides 3-(3-fluoro-4-(3-(piperidin-4-yl)propoxy)phenyl)-1-methyl-1H-pyrazolo[3,4-d]pyrimidin-6-amine in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides 3-(4-(((1r,4r)-4-aminocyclohexyl)methoxy)-3-fluorophenyl)-1-methyl-1H-pyrazolo[3,4-d]pyrimidin-6-amine in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides 3-(4-((4-aminobicyclo[2.2.2]octan-1-yl)methoxy)-3-fluorophenyl)-1-methyl-1H-pyrazolo[3,4-d]pyrimidin-6-amine in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides 3-(4-((4-aminobicyclo[2.2.2]octan-1-yl)methoxy)-3-chlorophenyl)-1-methyl-1H-pyrazolo[3,4-d]pyrimidin-6-amine in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

In one embodiment, the present invention provides 4-((2-chloro-4-(6-methoxy-1-methyl-1H-pyrazolo[3,4-d]pyrimidin-3-yl)phenoxy)methyl)bicyclo[2.2.2]octan-1-amine in an acetate, adipate, ascorbate, aspartate, benzoate, besylatye, benzenesulfonate, bicarbonate/carbonate, bisulfate/sulfate, bromide/hydrobromide, camphor sulfonate, camsylate, caprate, chloride/hydrochloride, chlortheophyllonate, citrate, edisylate, ethanedisulfonate, fumarate, gluceptate, glucoheptonate, gluconate, glucuronate, glutamate, glutarate, glycolate, hippurate, hydroiodide/iodide, isethionate, lactate, lactobionate, laurylsulphate, malate, maleate, malonate, mandelate, mesylate, methanesulfonate, methylsulfate, mucate, naphthoate, napsylate, 2-napsylate, naphthalenesulfonate, 2-naphthalenesulfonate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate, p-toluenesulfonate, trifluoroacetate, trifenatate, triphenylacetete or xinafoate salt form.

Lists of additional suitable acid addition salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

In certain embodiments, compounds of Formula (I) and subformulae thereof, are prepared or formed, as solvates (e.g., hydrates). In certain embodiments, hydrates of compounds of Formula (I) and subformulae thereof, are prepared by recrystallization from an aqueous/organic solvent mixture, using organic solvents such as dioxin, tetrahydrofuran or methanol. Furthermore, the compounds of the present invention, including their salts, can also be obtained in the form of their hydrates, or include other solvents used for their crystallization.

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

In certain embodiments, compounds of Formula (I), or subformulae thereof, are prepared as their individual stereoisomers. In other embodiments, the compounds of Formula (I), or subformulae thereof, are prepared as their individual stereoisomers by reacting a racemic mixture of the compound with an optically active resolving agent to form a pair of diastereoisomeric compounds, separating the diastereomers and recovering the optically pure enantiomers. In certain embodiments, resolution of enantiomers is carried out using covalent diastereomeric derivatives of the compounds of Formula (I), or subformulae thereof, or by using dissociable complexes (e.g., crystalline diastereomeric salts). Diastereomers have distinct physical properties (e.g., melting points, boiling points, solubility, reactivity, etc.) and are readily separated by taking advantage of these dissimilarities. In certain embodiments, the diastereomers are separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Depending on the choice of the starting materials and procedures, certain embodiments of the compounds of the present invention are present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Intermediates and final products can be worked up and/or purified according to standard methods, e.g. using chromatographic methods, distribution methods, (re-) crystallization, and the like. The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ. All starting materials, building blocks, reagents, acids, bases, dehydrating agents, solvents and catalysts utilized to synthesize the compounds of the present invention are either commercially available or can be produced by organic synthesis methods known to one of ordinary skill in the art.

Compounds of Formula (I) and subformulae thereof (Formula (Ia) and Formula (Ib)) are made by processes described in the general schemes herein and as illustrated in the Examples.

Scheme 1A illustrates a non-limiting synthetic scheme used to make certain compounds of Formula (A1) where the -linker-R⁴ moiety is attached to intermediate (int-A1) by an amide bond. In Scheme 1A the linker is any linker (L′) having a terminal carbonyl moiety (i.e. -L′—C(═O)). Also in Scheme 1A, R¹ is as described herein and R⁴ is a reactive moiety which can react with a thiol, a disulfide, an amine, a ketone, a diketone, an azide or an alkyne. Scheme 1B illustrates a non-limiting synthetic scheme used to make certain compounds of Formula (A1) where the -linker-R⁴ moiety is attached to intermediate (int-A1) by an amide bond. In Scheme 1B the linker is any linker (L′) having a terminal carbonyl moiety (i.e. -L′—C(═O)). Also in Scheme 1B, R¹ is as described herein and R⁴ moiety having an amino group (such as a hydroxyl amine or an amine) and R^{B} is moiety having a protected amino group, where Prot is a protecting group such as Boc, Fmoc and Cbz.

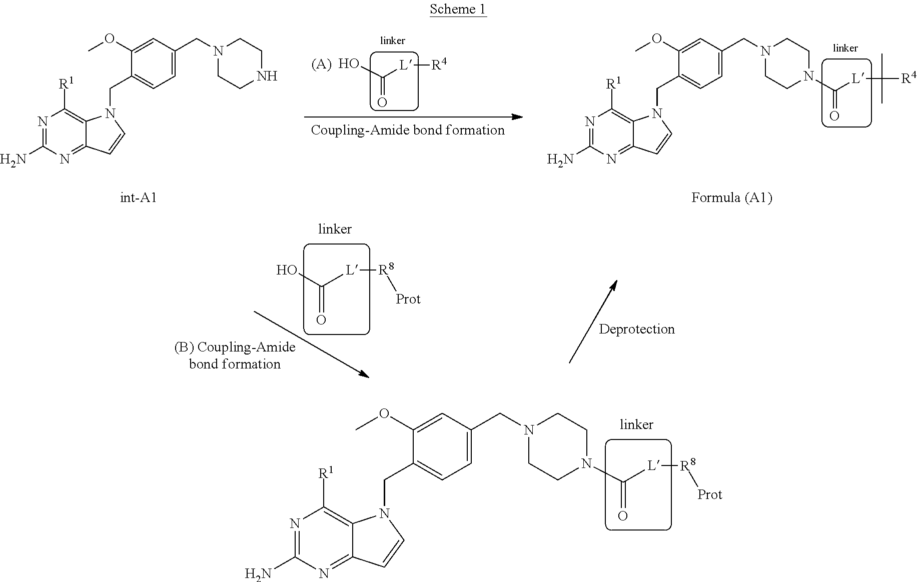

Such amide bond formation can be accomplished using heat, EDCl coupling, HATU coupling, HBTU coupling, TBTU coupling or T3P coupling.

Scheme 2A illustrates a non-limiting synthetic scheme used to make certain compounds of Formula (A2) where the -linker-R⁴ moiety is attached to intermediate (int-A2) by an amide bond. In Scheme 2A the linker is any linker (L′) having a terminal carbonyl moiety (i.e. -L′—C(═O)). Also in Scheme 2A, R¹ is as described herein and R⁴ is a reactive moiety which can react with a thiol, a disulfide, an amine, a ketone, a diketone, an azide or an alkyne. Scheme 2B illustrates a non-limiting synthetic scheme used to make certain compounds of Formula (A2) where the -linker-R⁴ moiety is attached to intermediate (int-A2) by an amide bond. In Scheme 2B the linker is any linker (L′) having a terminal carbonyl moiety (i.e. -L′—C(═O)). Also in Scheme 2B, R¹ is as described herein and R⁴ moiety having an amino group (such as a hydroxyl amine or an amine) and R^{B} is moiety having a protected amino group, where Prot is a protecting group such as Boc, Fmoc and Cbz.

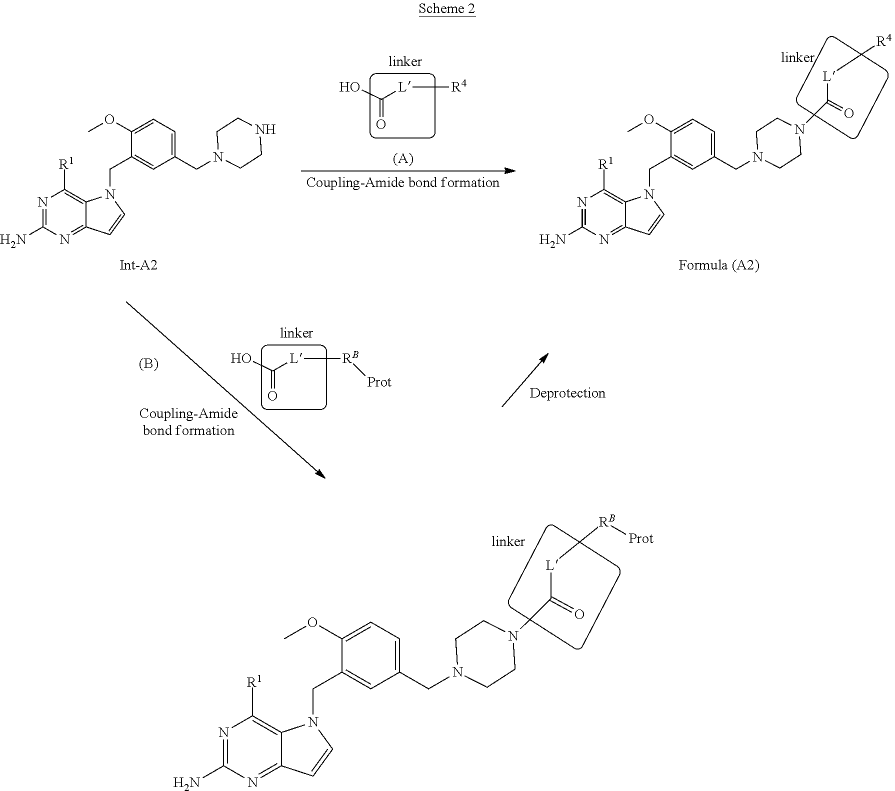

Such amide bond formation can be accomplished using heat, EDCl coupling, HATU coupling, HBTU coupling, TBTU coupling or T3P coupling.

Scheme 3A illustrates a non-limiting synthetic scheme used to make certain compounds of Formula (Ia) wherein the -L₂-R⁴ moiety is attached to intermediate (int-A1) by an amide bond. Such amide bond formation can be accomplished using heat, EDCl coupling, HATU coupling, HBTU coupling, TBTU coupling or T3P coupling. In Scheme 3A the linker (L₂) comprises a linker moiety (L_{A}) having a terminal carbonyl moiety (i.e. -L_{A}-C(═O)). Scheme 3B illustrates a non-limiting synthetic scheme used to make certain compounds of Formula (I) wherein the -L₂-R⁴ moiety is attached to intermediate (int-A1) by an amide bond. Such amide bond formation can be accomplished using heat, EDCl coupling, HATU coupling, HBTU coupling, TBTU coupling or T3P coupling. In Scheme 3B the linker (L₂) comprises a linker moiety (L_{A}) having a terminal carbonyl moiety (i.e. -L_{A}-C(═O)), and R^{B} is moiety having a protected amino group, where Prot is a protecting group such as Boc, Fmoc and Cbz.

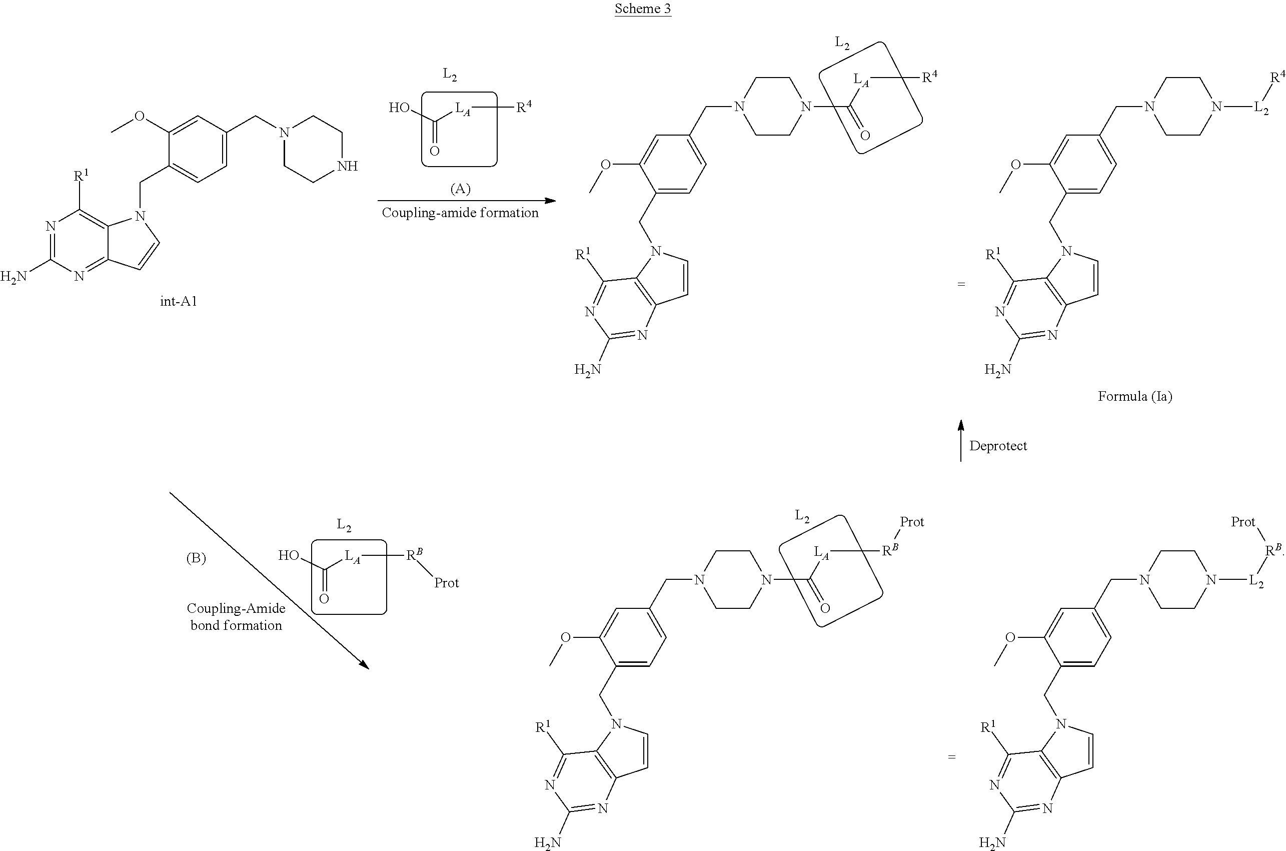

Scheme 4A illustrates a non-limiting synthetic scheme used to make certain compounds of Formula (Ib) wherein the -L₂-R⁴ moiety is attached to intermediate (int-A2) by an amide bond. Such amide bond formation can be accomplished using heat, EDCl coupling, HATU coupling, HBTU coupling, TBTU coupling or T3P coupling. In Scheme 4A the linker (L₂) comprises a linker moiety (L_{A}) having a terminal carbonyl moiety (i.e. -L_{A}-C(═O)). Scheme 4B illustrates a non-limiting synthetic scheme used to make certain compounds of Formula (Ib) wherein the -L₂-R⁴ moiety is attached to intermediate (int-A2) by an amide bond. Such amide bond formation can be accomplished using heat, EDCl coupling, HATU coupling, HBTU coupling, TBTU coupling or T3P coupling. In Scheme 4B the linker (1L₂) comprises a linker moiety (L_{A}) having a terminal carbonyl moiety (i.e. -L_{A}-C(═O)), and R^{B} is moiety having a protected amino group, where Prot is a protecting group such as Boc, Fmoc and Cbz.

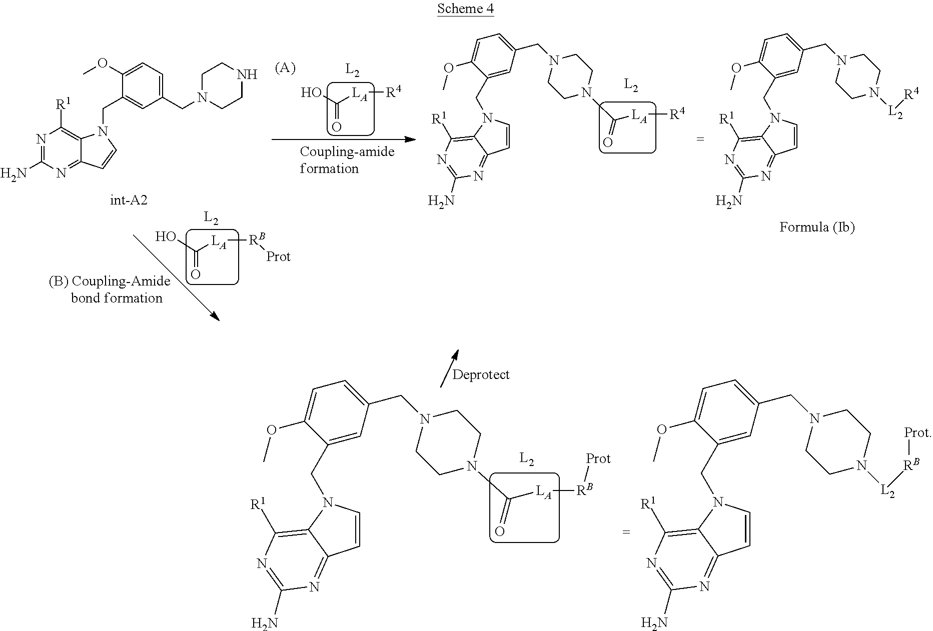

In Schemes 3 and 4,
R⁴

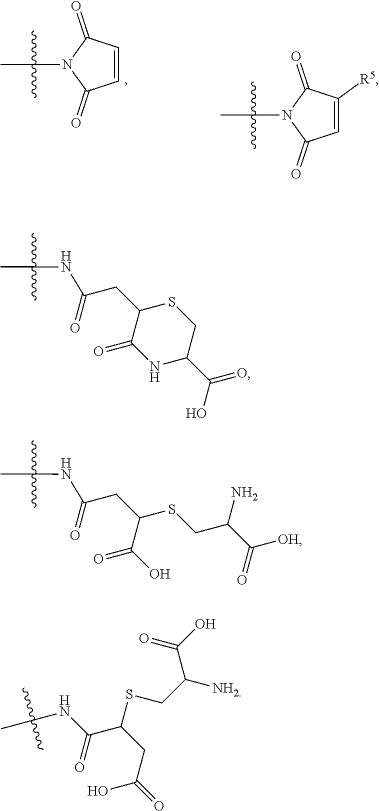

—ONH₂, —NH₂,

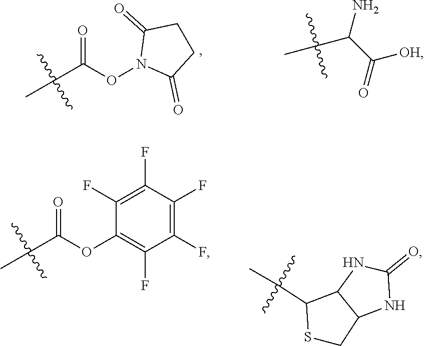 [SMILES: *C(=O)ON1C(=O)CCC1=O ; *C(N)C(=O)O ; *C(=O)Oc1c(F)c(F)c(F)c(F)c1F ; *C1SCC2NC(=O)NC21]

### —N₃,

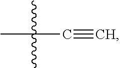 [SMILES: *C#C]

—NHC(═O)CH═CH₂, SH, —SSR⁶, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, —C(O)NHNH₂,

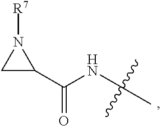

—CO₂H, —C(O)NHNH₂,

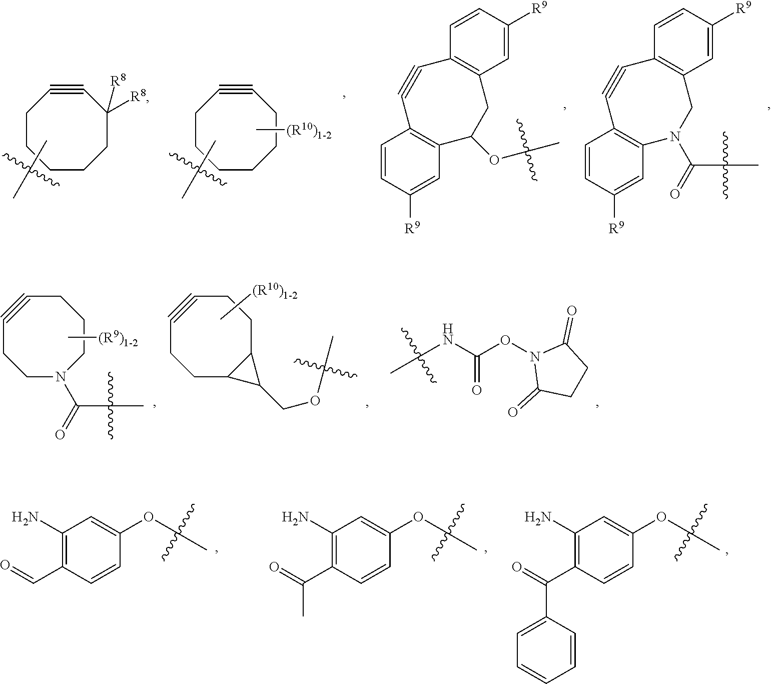

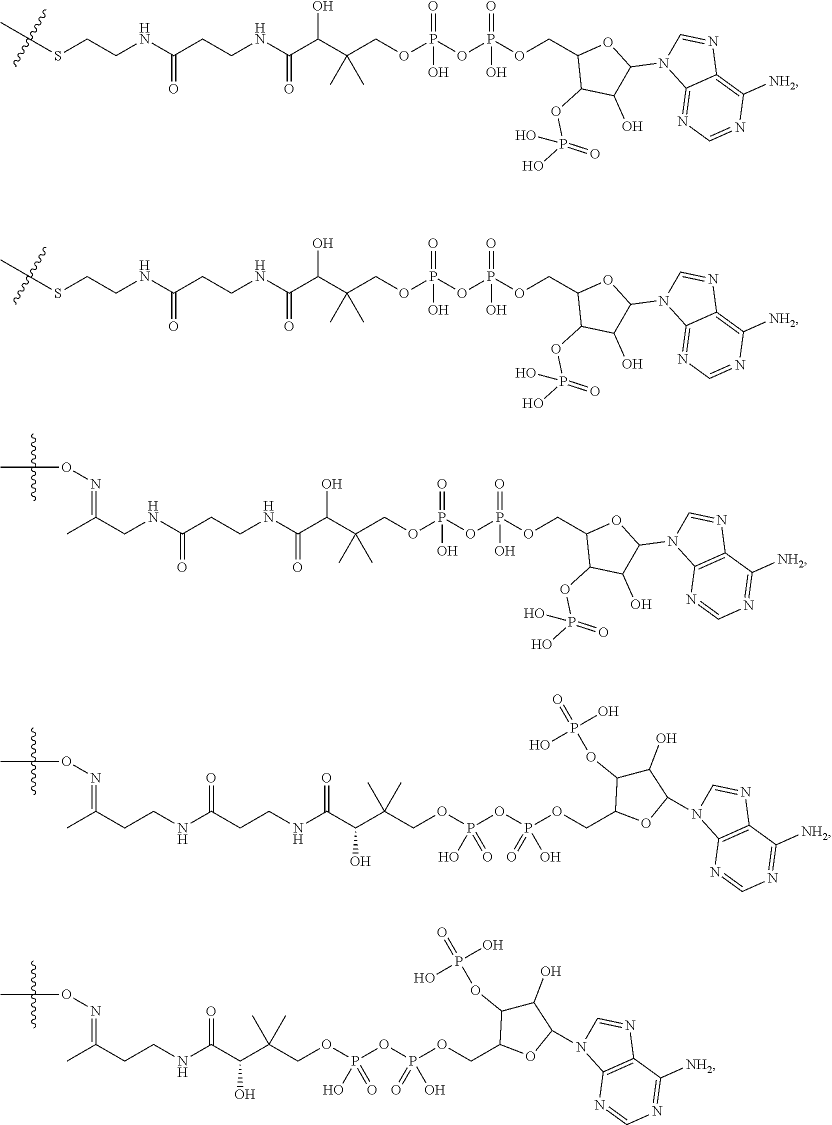 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CCNC(=O)CCNC(=O)[C@@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@@H](O)C(C)(C)COP(=O)(O)OP(=O)(O)OCC1OC(n2cnc3c(N)ncnc32)C(O)C1OP(=O)(O)O]

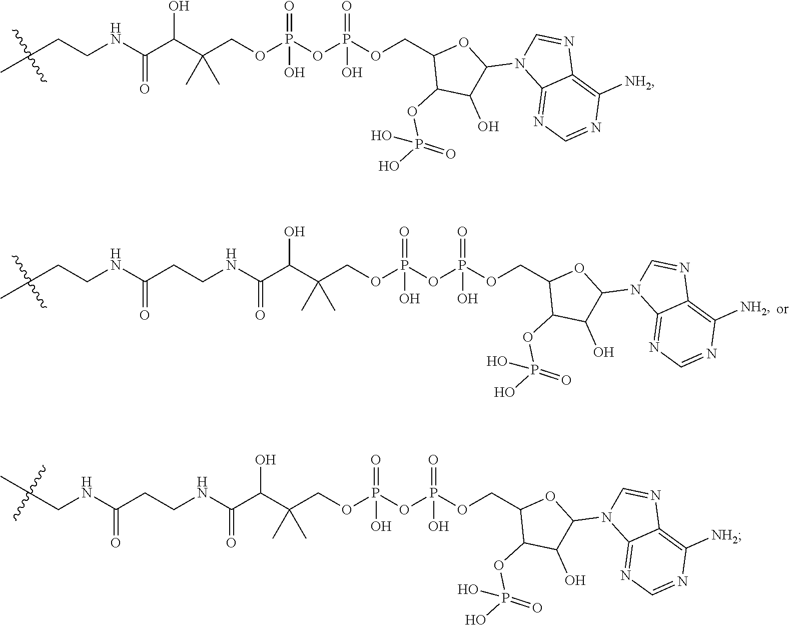 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

R^{B} is —ONH—;

L_{A} is —(CH₂)ₙ—, —((CH₂)ₙO)ₜ(CH₂)ₙ—, —((CH₂)ₙO)ₜ(CH₂)ₙX₁(CH₂)ₙ—, —((CH₂)ₙO)ₜ(CH₂)ₙNHC(═O)(CH₂)ₙ—, —((CH₂)ₙO)ₜ(CH₂)ₙC(═O)NH(CH₂)ₙ—, —NH((CH₂)ₙO)ₜ(CH₂)ₙX₁(CH₂)ₙ—, —X₂X₃C(═O)((CH₂)ₙO)ₜ(CH₂)ₙ—, —X₂C(═O)(CH₂)ₙNHC(═O)(CH₂)ₙ—, or —(CH₂)ₙC(═O)NH(CH₂)ₙ;
L₂ is —C(═O)(CH₂)ₙ—, —C(═O)((CH₂)ₙO)ₜ(CH₂)ₙ—, —C(═O)((CH₂)ₙO)ₜ(CH₂)ₙX₁(CH₂)ₙ—, —C(═O)((CH₂)ₙO)ₜ(CH₂)ₙNHC(═O)(CH₂)ₙ—, —C(═O)((CH₂)ₙO)ₜ(CH₂)ₙC(═O)NH(CH₂)ₙ—, —C(═O)NH((CH₂)ₙO)ₜ(CH₂)ₙX₁(CH₂)ₙ—, —C(═O)X₂X₃C(═O)((CH₂)ₙO)ₜ(CH₂)ₙ—, —C(═O)X₂C(═O)(CH₂)ₙNHC(═O)(CH₂)ₙ—, or —C(═O)(CH₂)ₙC(═O)NH(CH₂)ₙ;
   where X₁ is

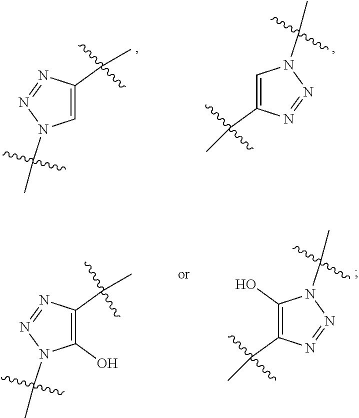 [SMILES: *c1cn(*)nn1 ; *c1cn(*)nn1 ; *c1nnn(*)c1O ; *c1nnn(*)c1O]

X₂ is

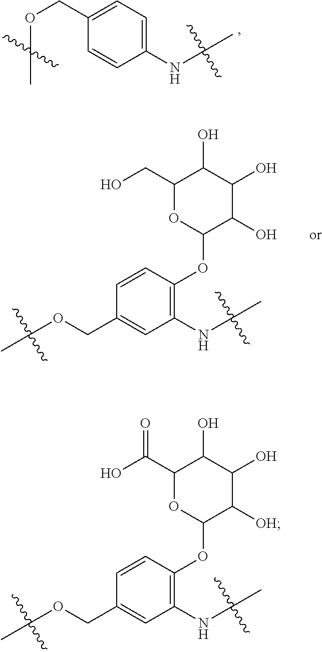 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

and X₃ is

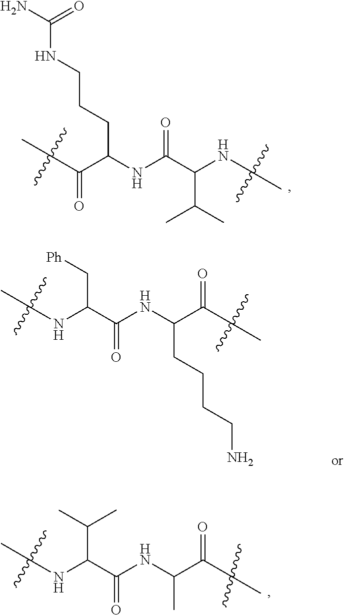 [SMILES: *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C]

and
R¹, R⁷, R⁸, R⁹ and R¹⁰, are as defined herein.

Scheme 5 illustrates a non-limiting synthetic scheme used to make certain compounds of Formula (B1) where the -linker-R⁴ moiety is attached to intermediate (int-A1) by alkylation of the secondary amine of intermediate (int-A1). In Scheme 5 the linker (L_{A}) is initially functionalized with a terminal aldehyde (i.e. -L_{A}-C(═O)H) and then reacted with the secondary amine of intermediate (int-A1). Also in Scheme 5, R¹ is as described herein and R⁴ is a reactive moiety which can react with a thiol, a disulfide, an amine, a ketone, a diketone, an azide or an alkyne.

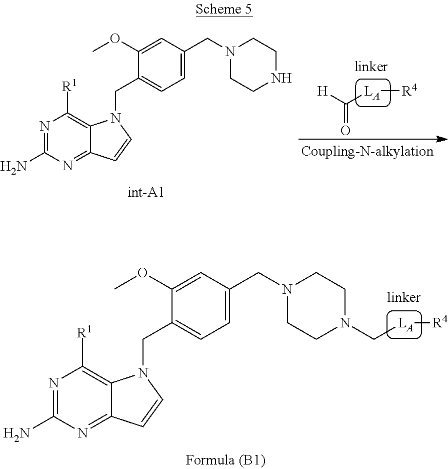

Such N-alkylation can be accomplished using a reducing agent such as NaCNBH₃, NaBH₄ or NaBH(OAC)₃.

Scheme 6 illustrates a non-limiting synthetic scheme used to make certain compounds of Formula (B2) where the -linker-R⁴ moiety is attached to intermediate (int-A2) by alkylation of the secondary amine of intermediate (int-A2). In Scheme 6 the linker (L_{A}) is initially functionalized with a terminal aldehyde (i.e. -L_{A}-C(═O)H) and then reacted with the secondary amine of intermediate (int-A2). Also in Scheme 6, R¹ is as described herein and R⁴ is a reactive moiety which can react with a thiol, a disulfide, an amine, a ketone, a diketone, an azide or an alkyne.

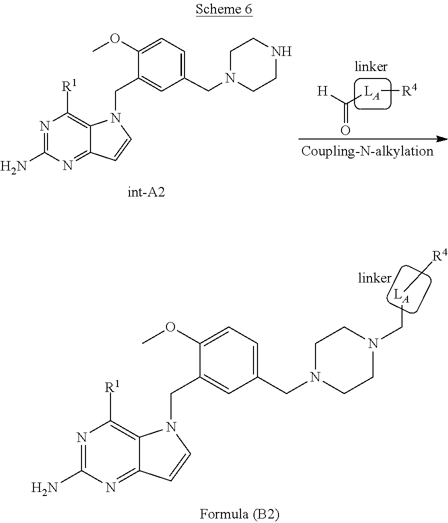

Such N-alkylation can be accomplished using a reducing agent such as NaCNBH₃, NaBH₄ or NaBH(OAC)₃.

Scheme 7 illustrates a non-limiting synthetic scheme used to make certain compounds of Formula (Ia) wherein the -L₂-R⁴ moiety is attached to intermediate (int-A1) by alkylation of the secondary amine of intermediate (int-A1). In Scheme 7 the linker moiety, L_{A}, initially functionalized with a terminal aldehyde (i.e. -L′—C(═O)H) is then reacted with the secondary amine of intermediate (int-A1), thereby forming the linker, L₂, which comprises the linker moiety L_{A} with a terminal —CH₂— group. Such N-alkylation can be accomplished using a reducing agent such as NaCNBH₃, NaBH₄ or NaBH(OAC)₃.

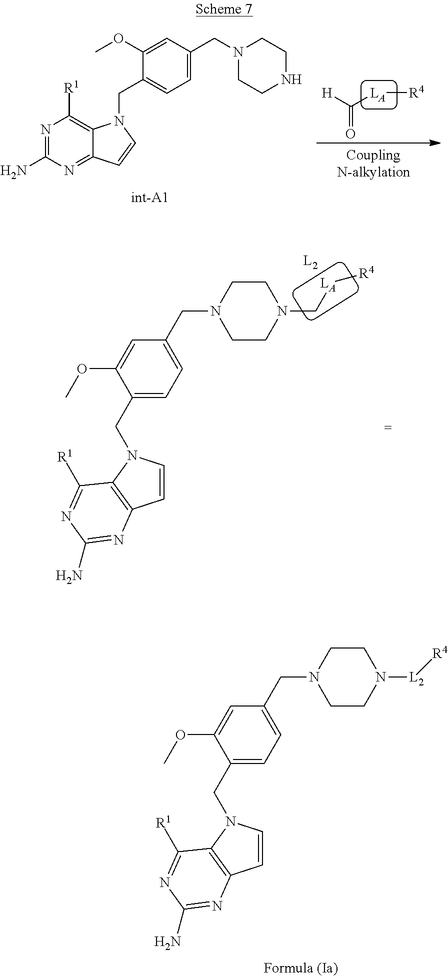

Scheme 8 illustrates a non-limiting synthetic scheme used to make certain compounds of Formula (Ib) wherein the -L₂-R⁴ moiety is attached to intermediate (int-A2) by alkylation of the secondary amine of intermediate (int-A2). In Scheme 8 the linker moiety (L_{A}) initially functionalized with a terminal aldehyde (i.e. -L′—C(═O)H) which is then reacted with the secondary amine of intermediate (int-A2), thereby forming the linker, L₂, which comprises the linker moiety L_{A} with a terminal —CH₂— group. Such N-alkylation can be accomplished using a reducing agent such as NaCNBH₃, NaBH₄ or NaBH(OAC)₃.

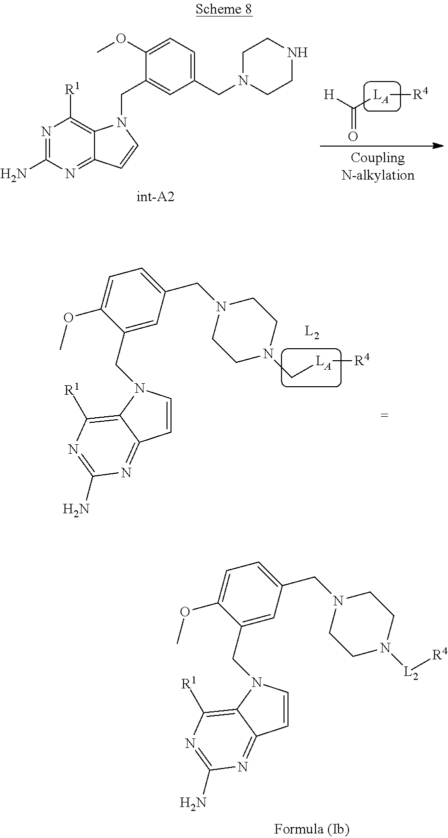

In Schemes 7 and 8,
R⁴ is as defined for Schemes 3 and 4;
L_{A} is —(CH₂)₍ₙ₋₁₎—, —((CH₂)₍ₙ₋₁₎O)((CH₂)ₙO)ₜ(CH₂)ₙ—, —(CH₂)₍ₙ₋₁₎X₁(CH₂)ₙ—, —(CH₂)₍ₙ₋₁₎NHC(═O)(CH₂)ₙ—, —(CH₂)₍ₙ₋₁₎NHC(═O(CH₂)ₙC(═O)NH(CH₂)ₙ— or —((CH₂)₍ₙ₋₁₎O)((CH₂)ₙO)ₜ(CH₂)ₙNHC(═O)(CH₂)ₙ;
L₂ is —(CH₂)ₙ—, —((CH₂)ₙO)ₜ(CH₂)ₙ—, —(CH₂)ₙX₁(CH₂)ₙ—, —(CH₂)ₙNHC(═O)(CH₂)ₙ—, —(CH₂)ₙNHC(═O(CH₂)ₙC(═O)NH(CH₂)ₙ— or —((CH₂)ₙO)ₜ(CH₂)ₙNHC(═O)(CH₂)ₙ;
   where X₁ is

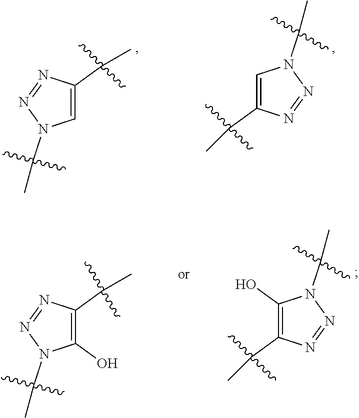 [SMILES: *c1cn(*)nn1 ; *c1cn(*)nn1 ; *c1nnn(*)c1O ; *c1nnn(*)c1O]

and
R¹ and R⁷ are as defined herein.

Scheme 9 illustrates a non-limiting synthetic scheme used to make certain compounds of Formula (A1) where the -linker-R⁴ moiety is attached to intermediate (int-A1) by an amide bond.

In Scheme 9 the linker is any linker (L′) having a terminal carbonyl moiety (i.e. -L′—C(═O)). Also in Scheme 9, R¹ is as described herein, R⁴ is

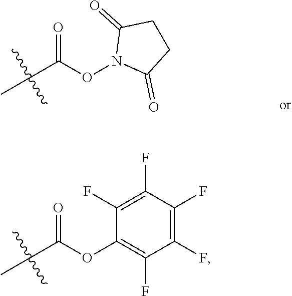 [SMILES: *C(=O)ON1C(=O)CCC1=O ; *C(=O)Oc1c(F)c(F)c(F)c(F)c1F]

### and R^{C} is

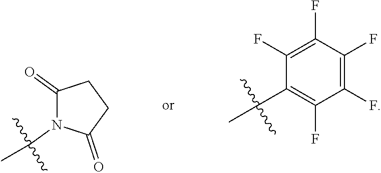 [SMILES: *N1C(=O)CCC1=O ; *c1c(F)c(F)c(F)c(F)c1F]

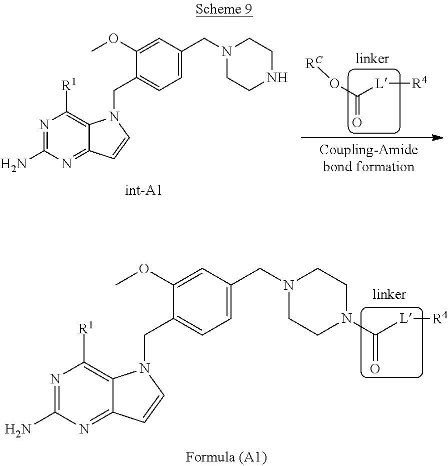

Such amide bond formation can be accomplished using heat, EDCl coupling, HATU coupling, HBTU coupling, TBTU coupling or T3P coupling.

Scheme 10 illustrates a non-limiting synthetic scheme used to make certain compounds of Formula (A2) where the -linker-R⁴ moiety is attached to intermediate (int-A2) by an amide bond. In Scheme 10 the linker is any linker (L′) having a terminal carbonyl moiety (i.e. -L′—C(═O)). Also in Scheme 10, R¹ is as described herein, R⁴ is

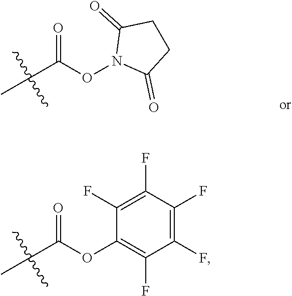 [SMILES: *C(=O)ON1C(=O)CCC1=O ; *C(=O)Oc1c(F)c(F)c(F)c(F)c1F]

### and R^{C} is

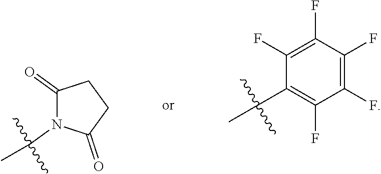 [SMILES: *N1C(=O)CCC1=O ; *c1c(F)c(F)c(F)c(F)c1F]

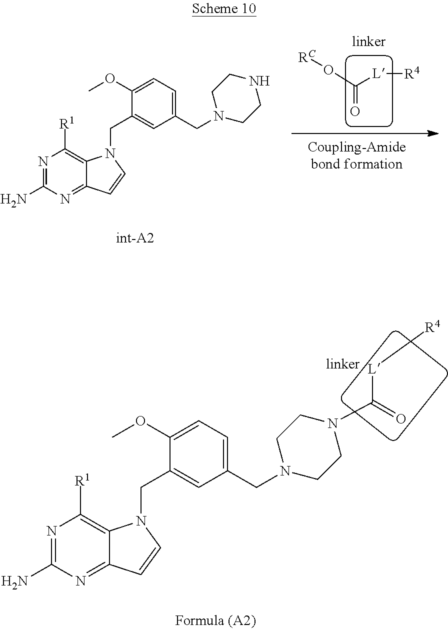

Such amide bond formation can be accomplished using heat, EDCl coupling, HATU coupling, HBTU coupling, TBTU coupling or T3P coupling.

Scheme 11 illustrates a non-limiting synthetic scheme used to make certain compounds of Formula (Ia) wherein the -L₂-R⁴ moiety is attached to intermediate (int-A1) by an amide bond. In Scheme 11 the linker (L₂) comprises a linker moiety (L_{A}) having a terminal carbonyl moiety (i.e. -L_{A}-C(═O)). Such amide bond formation can be accomplished using heat, EDCl coupling, HATU coupling, HBTU coupling, TBTU coupling or T3P coupling.

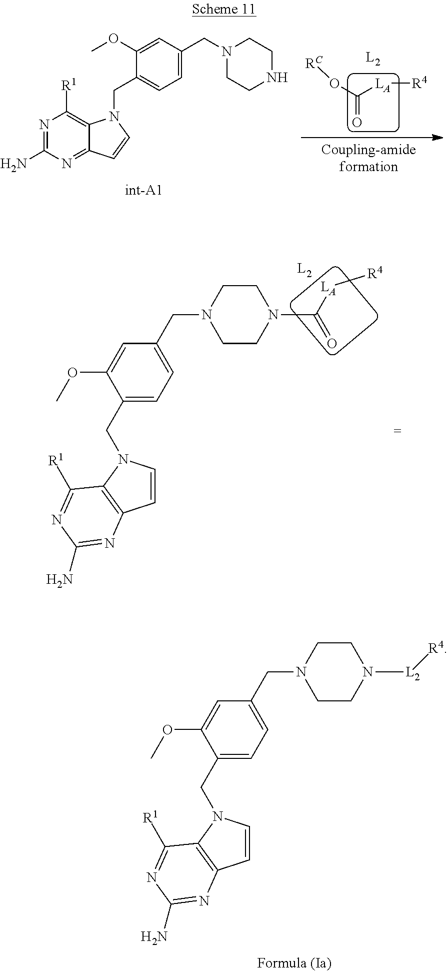

Scheme 12 illustrates a non-limiting synthetic scheme used to make certain compounds of Formula (Ib) wherein the -L₂-R⁴ moiety is attached to intermediate (int-A2) by an amide bond. In Scheme 12 the linker (L₂) comprises a linker moiety (L_{A}) having a terminal carbonyl moiety (i.e. -L_{A}-C(═O)). Such amide bond formation can be accomplished using heat, EDCl coupling, HATU coupling, HBTU coupling, TBTU coupling or T3P coupling.

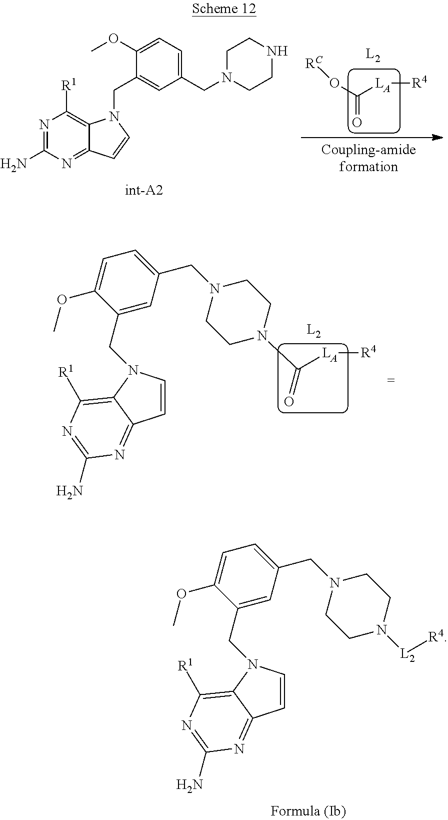

In Schemes 11 and 12,
R⁴ is

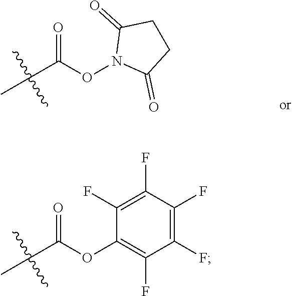 [SMILES: *C(=O)ON1C(=O)CCC1=O ; *C(=O)Oc1c(F)c(F)c(F)c(F)c1F]

R^{C} is

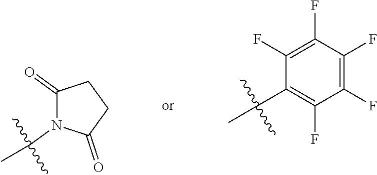 [SMILES: *N1C(=O)CCC1=O ; *c1c(F)c(F)c(F)c(F)c1F]

L_{A} is —(CH₂)ₙ—, —((CH₂)ₙO)ₜ(CH₂)ₙ—, —((CH₂)ₙO)ₜ(CH₂)ₙX₁(CH₂)ₙ—, —((CH₂)ₙO)ₜ(CH₂)ₙNHC(═O)(CH₂)ₙ—, —NH((CH₂)ₙO)ₜ(CH₂)ₙX₁(CH₂)ₙ—, —X₂X₃C(═O)((CH₂)ₙO)ₜ(CH₂)ₙ—, —X₂C(═O)(CH₂)ₙNHC(═O)(CH₂)ₙ—, or —(CH₂)ₙC(═O)NH(CH₂)ₙ—;
L₂ is —C(═O)(CH₂)ₙ—, —C(═O)((CH₂)ₙO)ₜ(CH₂)ₙ—, —C(═O)((CH₂)ₙO)ₜ(CH₂)ₙX₁(CH₂)ₙ—, —C(═O)((CH₂)ₙO)ₜ(CH₂)ₙNHC(═O)(CH₂)ₙ—, —C(═O)NH((CH₂)ₙO)ₜ(CH₂)ₙX₁(CH₂)ₙ—, —C(═O)X₂X₃C(═O)((CH₂)ₙO)ₜ(CH₂)ₙ—, —C(═O)X₂C(═O)(CH₂)ₙNHC(═O)(CH₂)ₙ—, or —C(═O)(CH₂)ₙC(═O)NH(CH₂)ₙ—; where X₁ is

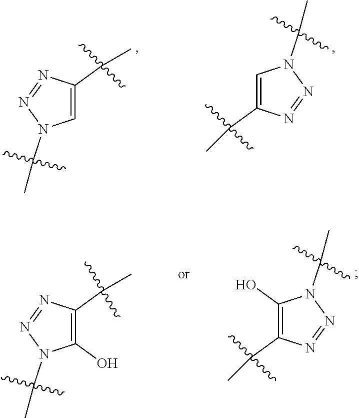 [SMILES: *c1cn(*)nn1 ; *c1cn(*)nn1 ; *c1nnn(*)c1O ; *c1nnn(*)c1O]

### X₂ is

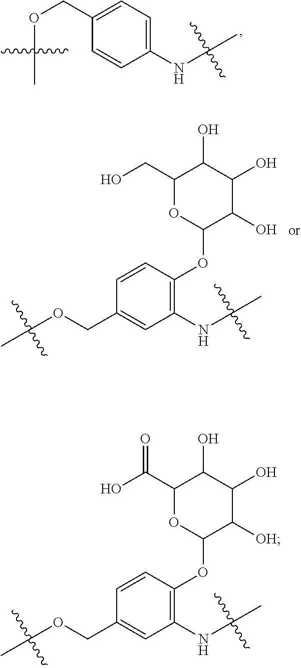 [SMILES: *Nc1ccc(CO*)cc1 ; *Nc1cc(CO*)ccc1OC1OC(CO)C(O)C(O)C1O ; *Nc1cc(CO*)ccc1OC1OC(C(=O)O)C(O)C(O)C1O]

### and X₃ is

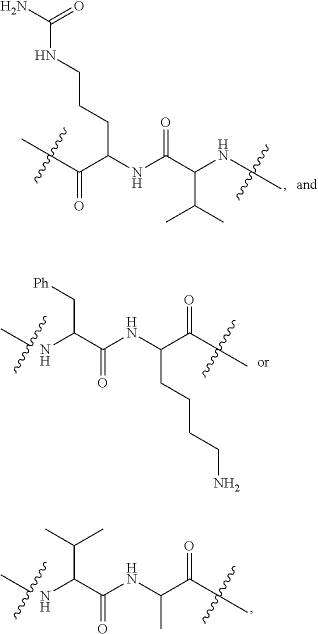 [1178] R¹ and R⁷ are as defined herein.

Compounds of Formula (IV) are made by processes described in Scheme 13 and as illustrated in the Examples.

Scheme 13 illustrates a non-limiting synthetic scheme used to make compounds of Formula (IV) where the

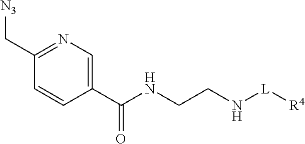

moiety is attached to an alkyne modified thymidine using Cu assisted “click chemistry.

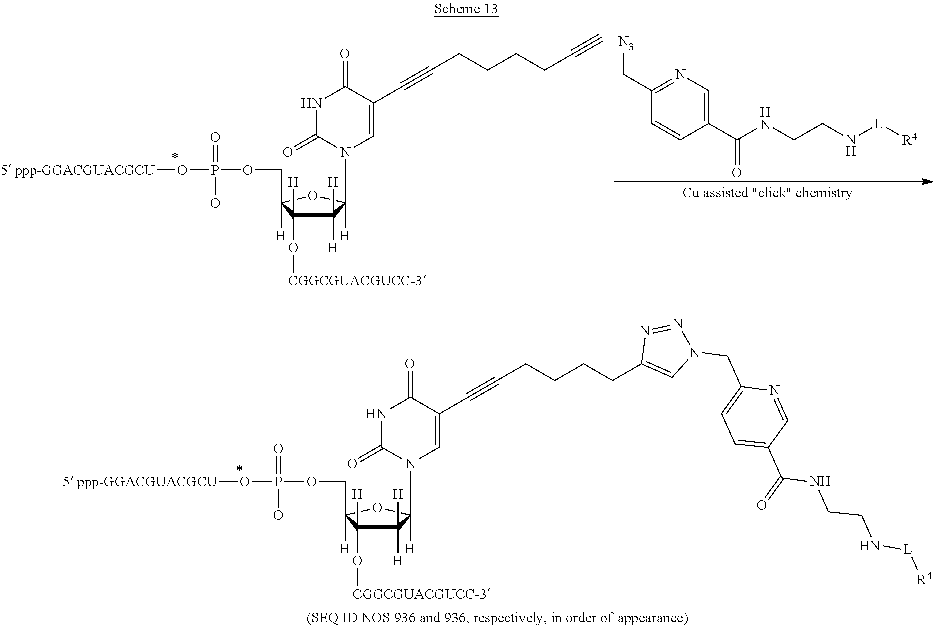

Scheme 14 illustrates a non-limiting synthetic scheme used to make compounds of Formula (IV) where the

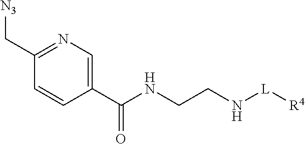

moiety is attached to an alkyne modified thymidine using Cu assisted “click chemistry.

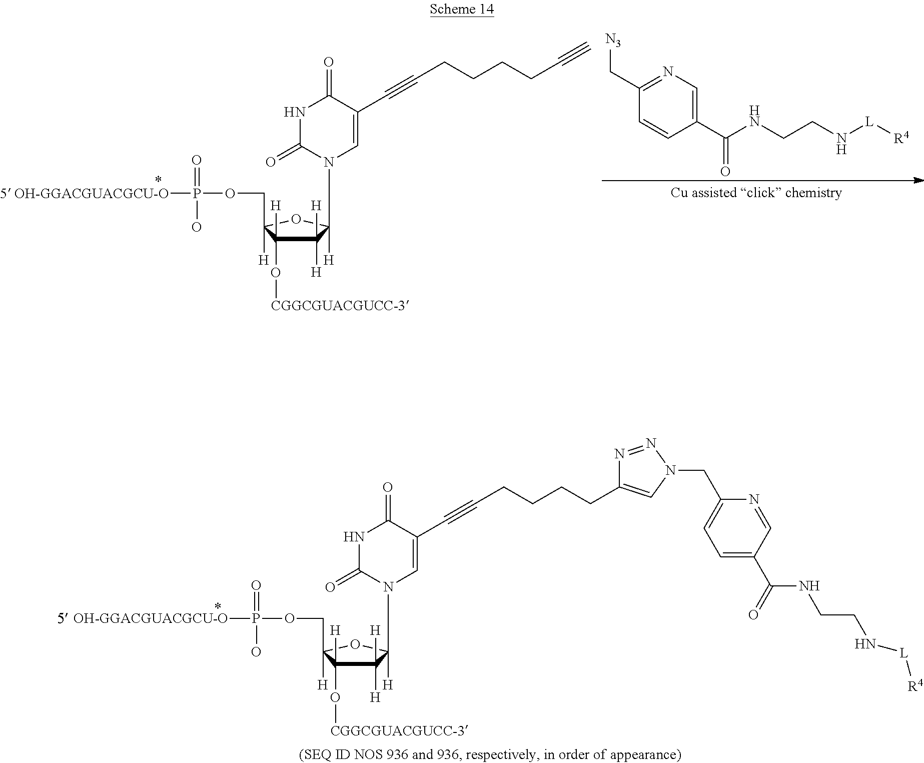

### Intermediates

The synthesis of the intermediates used to make the compounds of Formula (I) and subformulae thereof (i.e. compounds of Formula (Ia) and Formula (Ib)) of the invention are given below.

### Intermediate 1

### Synthesis of 5-(2-methoxy-4-(piperazin-1-ylmethyl)benzyl)-N4-pentyl-5H-pyrrolo[3,2-d]pyrimidine-2,4-diamine (Int-1)

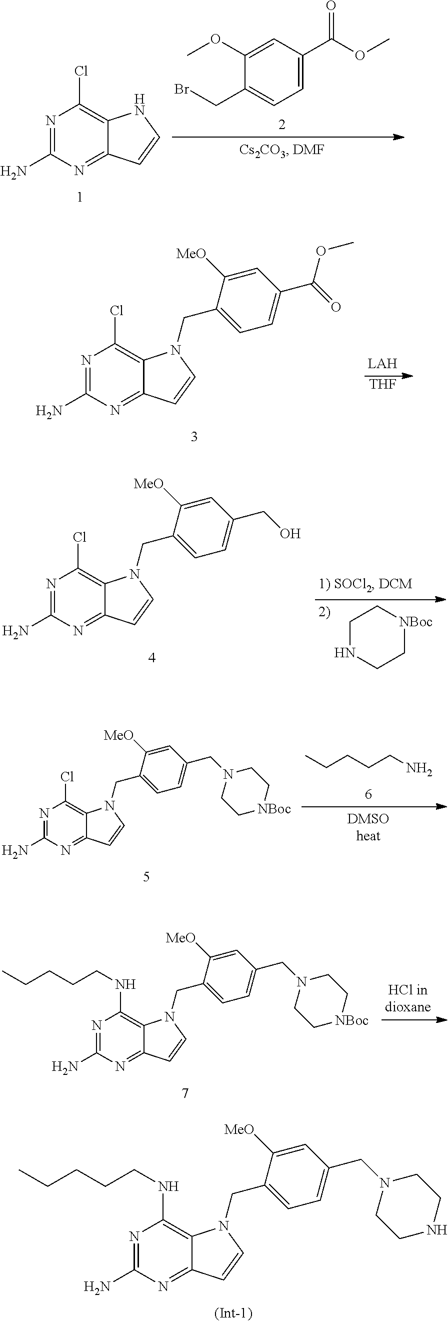

### Step 1: Preparation of methyl 4-((2-amino-4-chloro-5H-pyrrolo[3,2-d]pyrimidin-5-yl)methyl)-3-methoxybenzoate (3)

A round bottom flask was charged with 4-chloro-5H-pyrrolo[3,2-d]pyrimidin-2-amine (1, commercially available, 1.0 equiv.), methyl 4-(bromomethyl)-3-methoxybenzoate (2, commercially available, 1.0 equiv.), caesium carbonate (1.0 equiv.) and DMF (1.0 M). The reaction mixture was stirred at room temperature for 18 hours and the solvent was then removed in vacuo. To the resulting mixture was added EtOAc and the solvent was removed in vaccuo. To this mixture was added DCM and the solvent removed in vacuo. The crude reaction mixture was then purified by ISCO chromatography (0-10% MeOH:DCM, gradient) to afford methyl 4-((2-amino-4-chloro-5H-pyrrolo[3,2-d]pyrimidin-5-yl)methyl)-3-methoxybenzoate (3) as a solid.

### Step 2: (4-((2-amino-4-chloro-5H-pyrrolo[3,2-d]pyrimidin-5-yl)methyl)-3-methoxyphenyl)methanol (4)

A slurry of lithium aluminum, hydride (LAH) (1.0 equiv., powder) in THF (0.3 M) was prepared in a round bottom flask, cooled to 0° C. and vigorously stirred for 15 minutes. To this mixture was added methyl 4-((2-amino-4-chloro-5H-pyrrolo[3,2-d]pyrimidin-5-yl)methyl)-3-methoxybenzoate (3, 1.0 equiv. from previous step) in portions. The ice bath was removed and the reaction mixture was stirred at room temperature for 4 hours, with additional LAH being added until the reaction was complete). Et₂O was added to the reaction mixture and the mixture then transferred to an Erlenmeyer flask and cooled to 0° C. under vigorously stirring. The reaction was then quenched by the slow addition of a saturated sodium sulfate solution. A white precipitate was obtained and the mixture was filtered through a frit containing Celite and washed with THF and Et₂O. The volatiles were then removed in vacuo and the material used in the next step without further purification.

### Step 3: tert-butyl 4-(4-((2-amino-4-chloro-5H-pyrrolo[3,2-d]pyrimidin-5-yl)methyl)-3-methoxybenzyl)piperazine-1-carboxylate (5)

Thionyl chloride (10.0 equiv.) was added to a round bottom flask containing (4-((2-amino-4-chloro-5H-pyrrolo[3,2-d]pyrimidin-5-yl)methyl)-3-methoxyphenyl)methanol (4, 1.0 equiv. from step 2) in DCM (0.1 M) at 0° C. The ice-bath was then removed and the reaction mixture was stirred at room temperature for 4 hours. The reaction mixture was then cooled back to 0° C. and slowly quenched by the addition of NaOH (1.0 M, 40.0 equiv.) and saturated NaHCO₃ (aq.). The material was transferred to a separatory funnel and washed with DCM 3×. The combined organic layers were dried with sodium sulfate, filtered and volatiles removed in vacuo. The resulting crude product was then dissolved in DMF (0.1 M) in a round bottom flask and used without further purification. To this material was added tert-butyl piperazine-1-carboxylate (1.0 equiv.) and Huenig's base (1.2 equiv.) and stirred at room temperature for 18 hours. The reaction mixture was then diluted with EtOAc, transferred to a separatory funnel and washed with saturated NaCl (aq.) 2× and water 2×. The combined organic layers were dried with sodium sulfate, filtered and volatiles removed in vacuo. The crude reaction mixture was purified by ISCO chromatography (0-10% MeOH:DCM, gradient) to afford tert-butyl 4-(4-((2-amino-4-chloro-5H-pyrrolo[3,2-d]pyrimidin-5-yl)methyl)-3-methoxybenzyl)piperazine-1-carboxylate (5) as a solid.

### Step 4: tert-butyl 4-(4-((2-amino-4-(pentylamino)-5H-pyrrolo[3,2-d]pyrimidin-5-yl)methyl)-3-methoxybenzyl)piperazine-1-carboxylate (7)

A round bottom flask was charged with tert-butyl 4-(4-((2-amino-4-chloro-5H-pyrrolo[3,2-d]pyrimidin-5-yl)methyl)-3-methoxybenzyl)piperazine-1-carboxylate (5, 1.0 equiv. from step 3), commercially available pentylamine (6, 3.0 equiv.), Huenig's base (5.0 equiv.) and DMSO (0.5 M). The reaction mixture was heated to 120° C. and stirred for 18 hours. The reaction mixture was then cooled to room temperature and water added. This mixture was then frozen and the majority of volatiles removed by lyophilization. The crude reaction mixture was purified by ISCO chromatography (0-10% MeOH (the MeOH contained 0.7 N NH₃):DCM, gradient) to afford tert-butyl 4-(4-((2-amino-4-(pentylamino)-5H-pyrrolo[3,2-d]pyrimidin-5-yl)methyl)-3-methoxybenzyl)piperazine-1-carboxylate (7) as a solid.

### Step 5: 5-(2-methoxy-4-(piperazin-1-ylmethyl)benzyl)-N4-pentyl-5H-pyrrolo[3,2-d]pyrimidine-2,4-diamine (Int-1)

HCl in dioxane (4.0 M, 20.0 equiv.) was added to a solution of tert-butyl 4-(4-((2-amino-4-(pentylamino)-5H-pyrrolo[3,2-d]pyrimidin-5-yl)methyl)-3-methoxybenzyl)piperazine-1-carboxylate (6, 1.0 equiv. from step 4) in DCM (0.1 M) in a round bottom flask at 0° C. The ice-bath was then removed and the reaction mixture was stirred at room temperature for 3 hours. NH₃ in MeOH (0.7 N) was then added to the reaction mixture and the volatiles removed in vacuo. The addition of NH₃ in MeOH (0.7 N) and removal of volatiles in vacuo was repeated two more times. The crude reaction mixture was then purified by ISCO chromatography (0-20% MeOH (the MeOH contained 0.7 N NH₃):DCM, gradient) to provide 5-(2-methoxy-4-(piperazin-1-ylmethyl)benzyl)-N4-pentyl-5H-pyrrolo[3,2-d]pyrimidine-2,4-diamine (Int-1) as a solid: ¹H NMR (CD₃OD): δ 7.37 (d, 1H), 7.10 (s, 1H), 6.91 (d, 1H), 6.74 (d, 1H), 6.22 (d, 1H), 5.52 (s, 2H), 3.92 (s, 3H), 3.61 (s, 2H), 3.54 (t, 2H), 3.35 (s, 3H), 3.22 (m, 4H), 2.69 (m, 4H), 1.51 (m, 2H), 1.30 (m, 2H), 1.18 (m, 2H), 0.89 (s, 3H). LRMS [M+H]=438.3.

### Intermediate 2

### Synthesis of (S)-2-((2-amino-5-(2-methoxy-5-(piperazin-1-ylmethyl)benzyl)-5H-pyrrolo[3,2-d]pyrimidin-4-yl)amino)hexan-1-ol (Int-2)

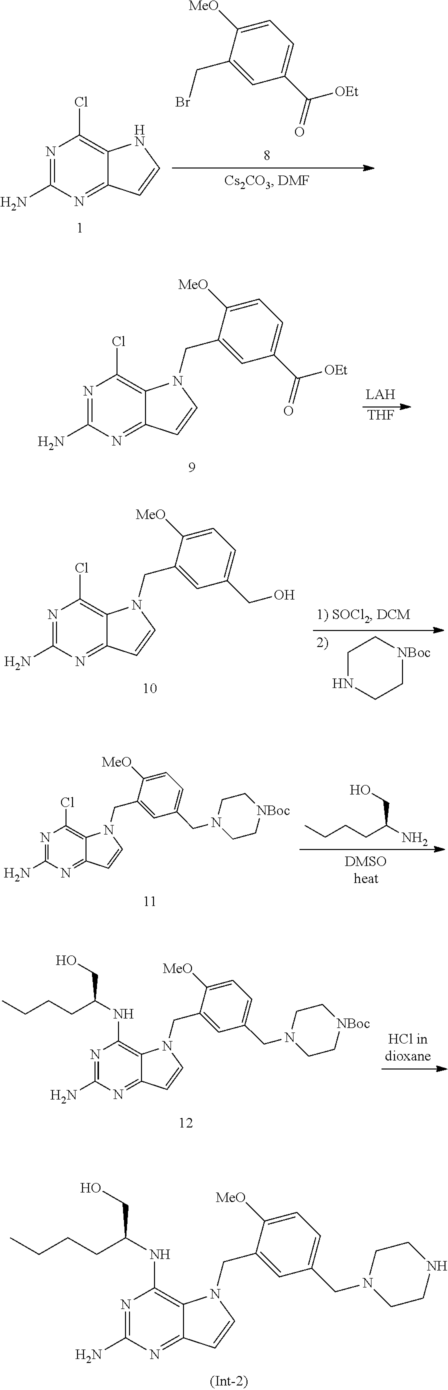

### Step 1: Preparation of ethyl 3-((2-amino-4-chloro-5H-pyrrolo[3,2-d]pyrimidin-5-yl)methyl)-4-methoxybenzoate (9)

A round bottom flask was charged with 4-chloro-5H-pyrrolo[3,2-d]pyrimidin-2-amine (1, commercially available, 1.0 equiv.), ethyl 3-(bromomethyl)-4-methoxybenzoate (8, commercially available, 1.0 equiv.), caesium carbonate (1.0 equiv.) and DMF (1.0 M). The reaction mixture was stirred at room temperature for 18 hours. The solvent was then removed in vaccuo. To the resulting mixture was added EtOAc and the solvent was removed in vacuo. To this mixture was added DCM and the solvent removed in vaccuo. The crude reaction mixture was then purified by ISCO chromatography (0-10% MeOH:DCM, gradient) to afford ethyl 3-((2-amino-4-chloro-5H-pyrrolo[3,2-d]pyrimidin-5-yl)methyl)-4-methoxybenzoate (9) as a solid.

### Step 2: (3-((2-amino-4-chloro-5H-pyrrolo[3,2-d]pyrimidin-5-yl)methyl)-4-methoxyphenyl)methanol (10)

A slurry of LAH (1.0 equiv., powder) in THF (0.3 M) was prepared in a round bottom flask, cooled to 0° C. and vigorously stirred for 15 minutes. To this mixture was added ethyl 3-((2-amino-4-chloro-5H-pyrrolo[3,2-d]pyrimidin-5-yl)methyl)-4-methoxybenzoate (9, 1.0 equiv. from step 1) in portions. The ice-bath was then removed and the reaction mixture was stirred at room temperature for 4 hours (if the reaction was not complete by this time additional LAH was added and stirring continued until the reaction was complete). The reaction mixture was then transferred to an Erlenmeyer flask using Et₂O. The mixture was cooled to 0° C. and vigorously stirred. The reaction was then quenched by the slow addition of a saturated sodium sulfate solution. A white precipitate was obtained and the mixture was filtered through a frit containing Celite and washed with THF and Et₂O. The volatiles were then removed in vacuo and the material used in the next step without further purification.

### Step 3: tert-butyl 4-(3-((2-amino-4-chloro-5H-pyrrolo[3,2-d]pyrimidin-5-yl)methyl)-4-methoxybenzyl)piperazine-1-carboxylate (11)

Thionyl chloride (10.0 equiv.) was added to a round bottom flask containing (3-((2-amino-4-chloro-5H-pyrrolo[3,2-d]pyrimidin-5-yl)methyl)-4-methoxyphenyl)methanol (10, 1.0 equiv. from step 2) in DCM (0.1 M) at 0° C. The ice-bath was then removed and the reaction mixture stirred at room temperature for 4 hours. The reaction mixture was then cooled to 0° C. and slowly quenched by the addition of NaOH (1.0 M, 40.0 equiv.) and saturated NaHCO₃ (aq.). The material was transferred to a separatory funnel and washed with DCM 3×. The combined organic layers were dried with sodium sulfate, filtered and volatiles removed in vacuo. The resulting crude product was then dissolved in DMF (0.1 M) in a round bottom flask and used without further purification. To this material was added tert-butyl piperazine-1-carboxylate (1.0 equiv.) and Huenig's base (1.2 equiv.) and stirred at room temperature for 18 hours. The reaction mixture was then diluted with EtOAc, transferred to a separatory funnel and washed with saturated NaCl (aq.) 2× and water 2×. The combined organic layers were dried with sodium sulfate, filtered and volatiles removed in vacuo. The crude reaction mixture was purified by ISCO chromatography (0-10% MeOH:DCM, gradient) to afford tert-butyl 4-(3-((2-amino-4-chloro-5H-pyrrolo[3,2-d]pyrimidin-5-yl)methyl)-4-methoxybenzyl)piperazine-1-carboxylate (11) as a solid.

### Step 4: (S)-tert-butyl 4-(3-((2-amino-4-((1-hydroxyhexan-2-yl)amino)-5H-pyrrolo[3,2-d]pyrimidin-5-yl)methyl)-4-methoxybenzyl)piperazine-1-carboxylate (12)

A round bottom flask was charged with tert-butyl 4-(3-((2-amino-4-chloro-5H-pyrrolo[3,2-d]pyrimidin-5-yl)methyl)-4-methoxybenzyl)piperazine-1-carboxylate (11, 1.0 equiv. from step 3), commercially available (S)-2-aminohexan-1-ol (3.0 equiv.), Huenig's base (5.0 equiv.) and DMSO (0.5 M). The reaction mixture was heated to 120° C. and stirred for 18 hours. The reaction mixture was then cooled to room temperature and water added. This mixture was then frozen and the majority of volatiles removed by lyophilization. The crude reaction mixture was purified by ISCO chromatography (0-10% MeOH (the MeOH contained 0.7 N NH₃):DCM, gradient) to afford (S)-tert-butyl 4-(3-((2-amino-4-((1-hydroxyhexan-2-yl)amino)-5H-pyrrolo[3,2-d]pyrimidin-5-yl)methyl)-4-methoxybenzyl)piperazine-1-carboxylate (12) as a solid.

### Step 5: Example 1-(S)-2-((2-amino-5-(2-methoxy-5-(piperazin-1-ylmethyl)benzyl)-5H-pyrrolo[3,2-d]pyrimidin-4-yl)amino)hexan-1-ol (Int-2)

HCl in dioxane (4.0 M, 20.0 equiv.) was added to a solution of (S)-tert-butyl 4-(3-((2-amino-4-((1-hydroxyhexan-2-yl)amino)-5H-pyrrolo[3,2-d]pyrimidin-5-yl)methyl)-4-methoxybenzyl)piperazine-1-carboxylate (12, 1.0 equiv. from step 4) in DCM (0.1 M) in a round bottom flask at 0° C. The ice-bath was then removed and the reaction mixture was stirred at room temperature for 3 hours. NH₃ in MeOH (0.7 N) was then added to the reaction mixture and the volatiles removed in vacuo. The addition of NH₃ in MeOH (0.7 N) and removal of volatiles in vacuo was repeated two more times. The crude reaction mixture was then purified by ISCO chromatography (0-20% MeOH (the MeOH contained 0.7 N NH₃):DCM, gradient) to provide (S)-2-((2-amino-5-(2-methoxy-5-(piperazin-1-ylmethyl)benzyl)-5H-pyrrolo[3,2-d]pyrimidin-4-yl)amino)hexan-1-ol (Int-2) as a solid: ¹H (CD₃OD): δ 7.50 (d, 1H), 7.29 (d, 1H), 7.09 (d, 1H), 6.63 (s, 1H), 6.29 (d, 1H), 5.69 (d, 1H), 5.40 (d, 1H), 4.34 (m, 1H), 3.95 (s, 3H), 3.51 (m, 2H), 3.42 (s, 2H), 3.12 (m, 4H), 2.56 (m, 2H), 1.48 (m, 1H), 1.21 (m, 3H), 0.96 (m, 2H), 0.83 (t, 3H). LRMS [M+H]=468.3.

### Intermediate 3

### Synthesis of 5-(2-methoxy-5-(piperazin-1-ylmethyl)benzyl)-N4-pentyl-5H-pyrrolo[3,2-d]pyrimidine-2,4-diamine (Int-3)

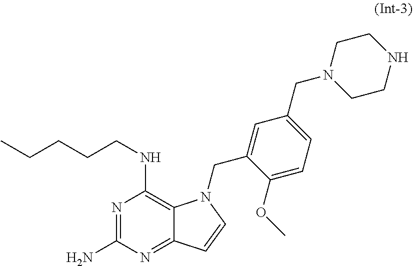 [SMILES: CCCCCNc1nc(N)nc2ccn(Cc3cc(CN4CCNCC4)ccc3OC)c12]

5-(2-methoxy-5-(piperazin-1-ylmethyl)benzyl)-N4-pentyl-5H-pyrrolo[3,2-d]pyrimidine-2,4-diamine (Int-3) was prepared according to the synthesis of (S)-2-((2-amino-5-(2-methoxy-5-(piperazin-1-ylmethyl)benzyl)-5H-pyrrolo[3,2-d]pyrimidin-4-yl)amino)hexan-1-ol (Int-2), except commercially available N-pentylamine was used in place of (S)-2-aminohexan-1-ol in Step 4. ¹H NMR (CD₃OD): δ 7.42 (d, 1H), 7.32 (d, 1H), 7.09 (d, 1H), 6.70 (s, 1H), 6.25 (d, 1H), 5.54 (d, 2H), 3.92 (s, 3H), 3.52 (t, 2H), 3.46 (s, 2H), 3.14 (m, 4H), 2.60 (m, 4H), 1.48 (m, 2H), 1.30 (m, 2H), 1.13 (m, 2H), 0.88 (t, 3H). LRMS [M+H]=438.3.

### Processes for Making Antibody Conjugate of Formula (IIa) and Formula (IIb)

A general reaction scheme for the formation of immunostimulatory conjugates of Formula (II) is shown in Scheme 15 below:

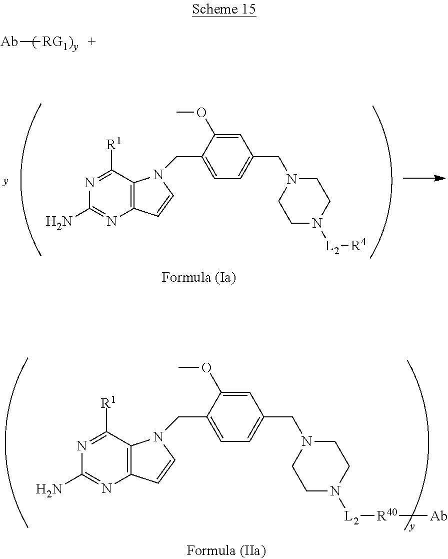

here: RG₁ is a reactive group which reacts with a compatible R⁴ group of a compound of Formula (Ia) to form a corresponding R⁴⁰ group, such as maleimide reacting with a thiol to give a succinimide ring, or a hydroxylamine reacting with a ketone to give an oxime; R¹, R⁴, L₂, Ab and R⁴⁰are as defined herein.

A general reaction scheme for the formation of immunostimulatory conjugates of Formula (IIb) is shown in Scheme 16 below:

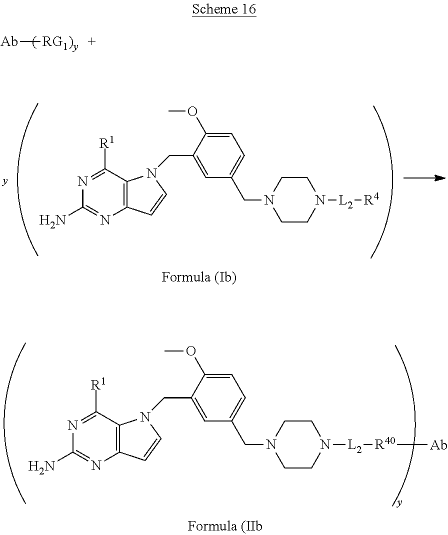

where: RG₁ is a reactive group which reacts with a compatible R⁴ group of a compound of Formula (Ib) to form a corresponding R⁴⁰ group, such as maleimide reacting with a thiol to give a succinimide ring, or a hydroxylamine reacting with a ketone to give an oxime; R¹, R⁴, L₂, Ab and R⁴⁰are as defined herein.

A general reaction scheme for the formation of immunostimulatory conjugates of Formula (V) is shown in Scheme 17 below:

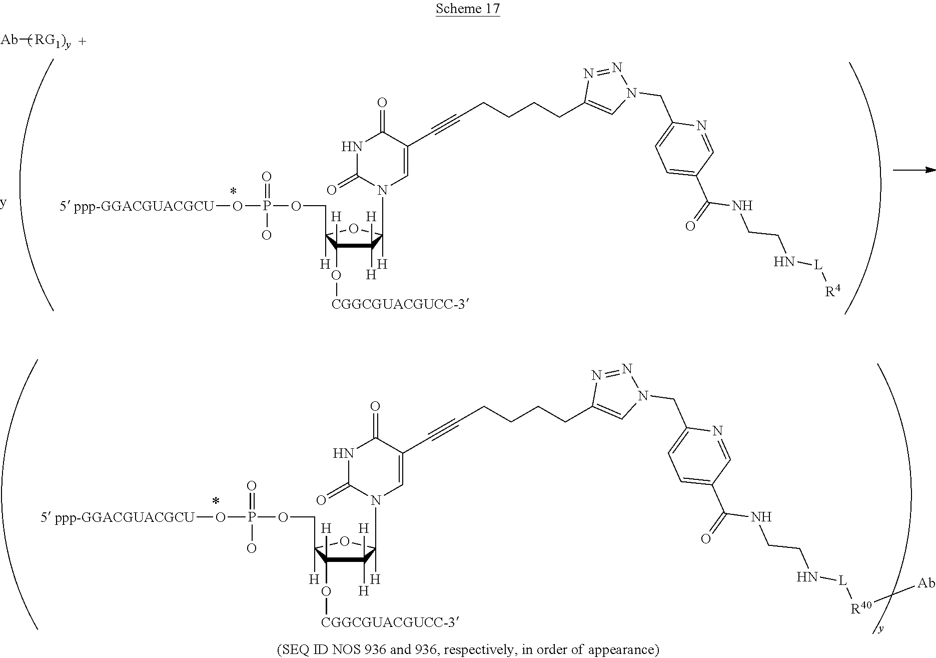

where: RG₁ is a reactive group which reacts with a compatible R⁴ group to form a corresponding R⁴⁰ group, such as maleimide reacting with a thiol to give a succinimide ring, or a hydroxylamine reacting with a ketone to give an oxime; y, L, Ab, R⁴ and R⁴⁰ are as defined herein for compounds of Formula (V).

A general reaction scheme for the formation of immunostimulatory conjugates of Formula (V) is shown in Scheme 18 below:

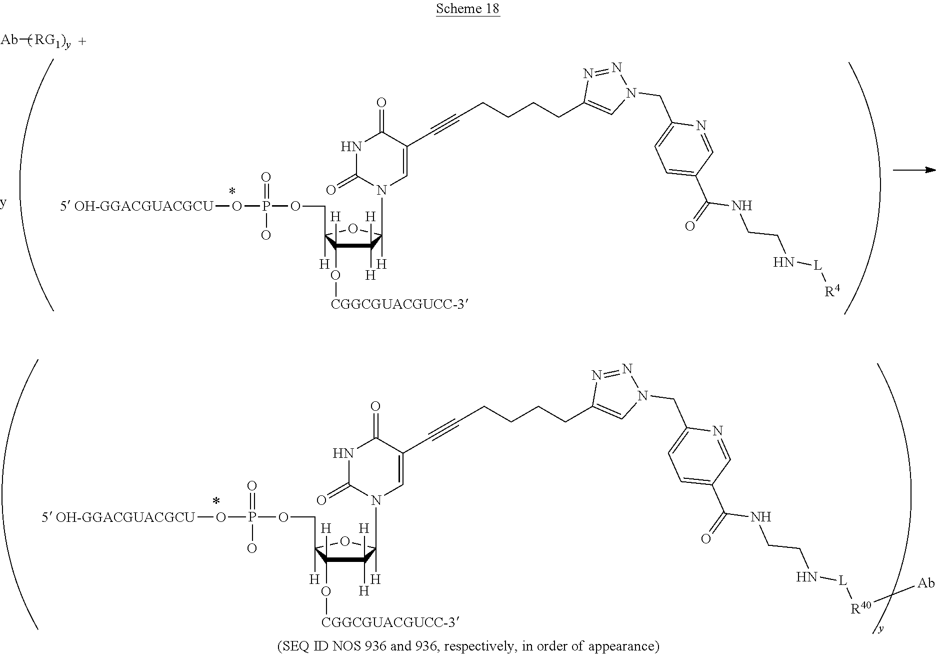

where: RG₁ is a reactive group which reacts with a compatible R⁴ group to form a corresponding R⁴⁰ group, such as maleimide reacting with a thiol to give a succinimide ring, or a hydroxylamine reacting with a ketone to give an oxime; y, L, Ab, R⁴ and R⁴⁰are as defined herein for compounds of Formula (V).

A general reaction scheme for the formation of immunostimulatory conjugates of Formula (V) is shown in Scheme 19 below:

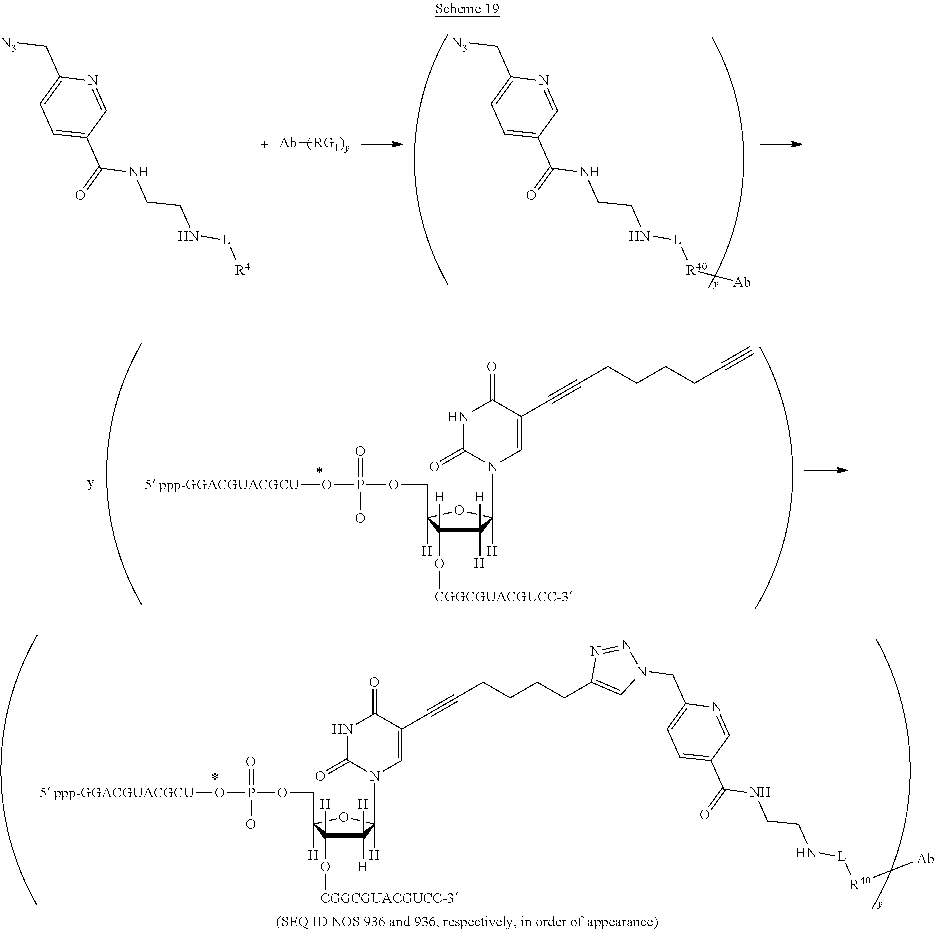

where: RG₁ is a reactive group which reacts with a compatible R⁴ group to form a corresponding R⁴⁰ group, such as maleimide reacting with a thiol to give a succinimide ring, or a hydroxylamine reacting with a ketone to give an oxime; y, L, Ab, R⁴ and R⁴⁰are as defined herein for compounds of Formula (V).

A general reaction scheme for the formation of immunostimulatory conjugates of Formula (V) is shown in Scheme 20 below:

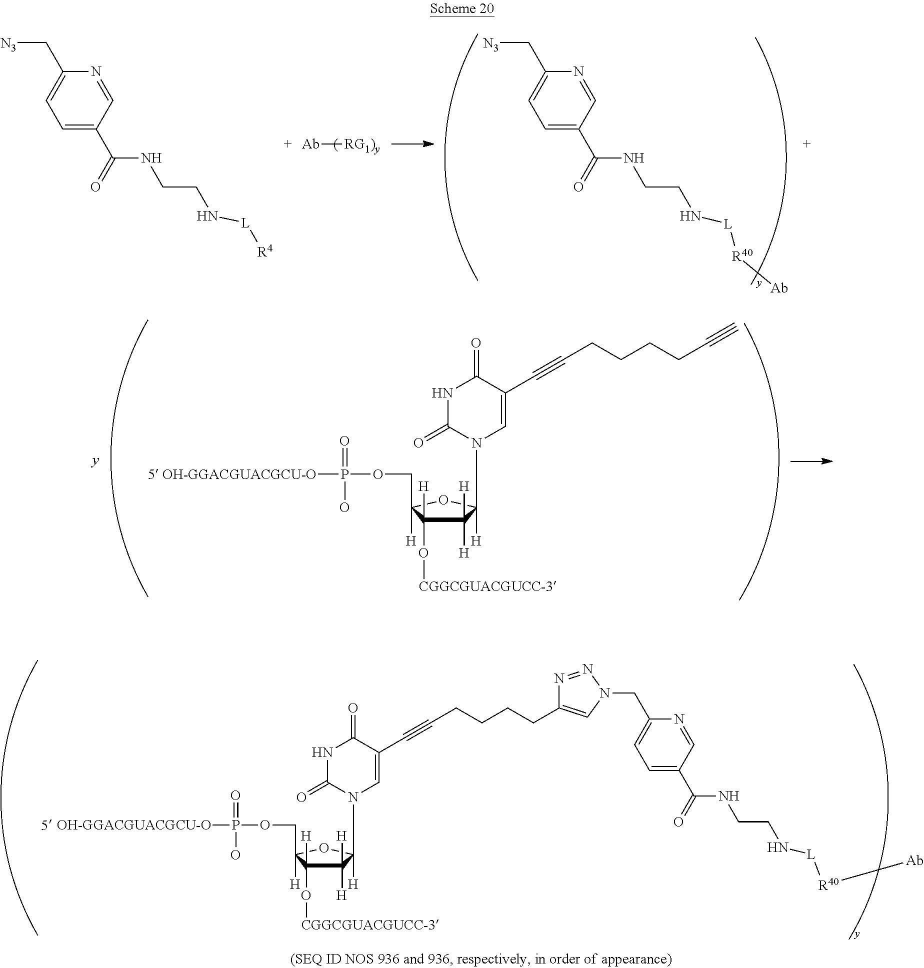

where: RG₁ is a reactive group which reacts with a compatible R⁴ group to form a corresponding R⁴⁰ group, such as maleimide reacting with a thiol to give a succinimide ring, or a hydroxylamine reacting with a ketone to give an oxime; y, L, Ab, R⁴ and R⁴⁰are as defined herein for compounds of Formula (V).

A general reaction scheme for the formation of certain immunostimulatory conjugates of Formula (V) is shown in Scheme 21 below:

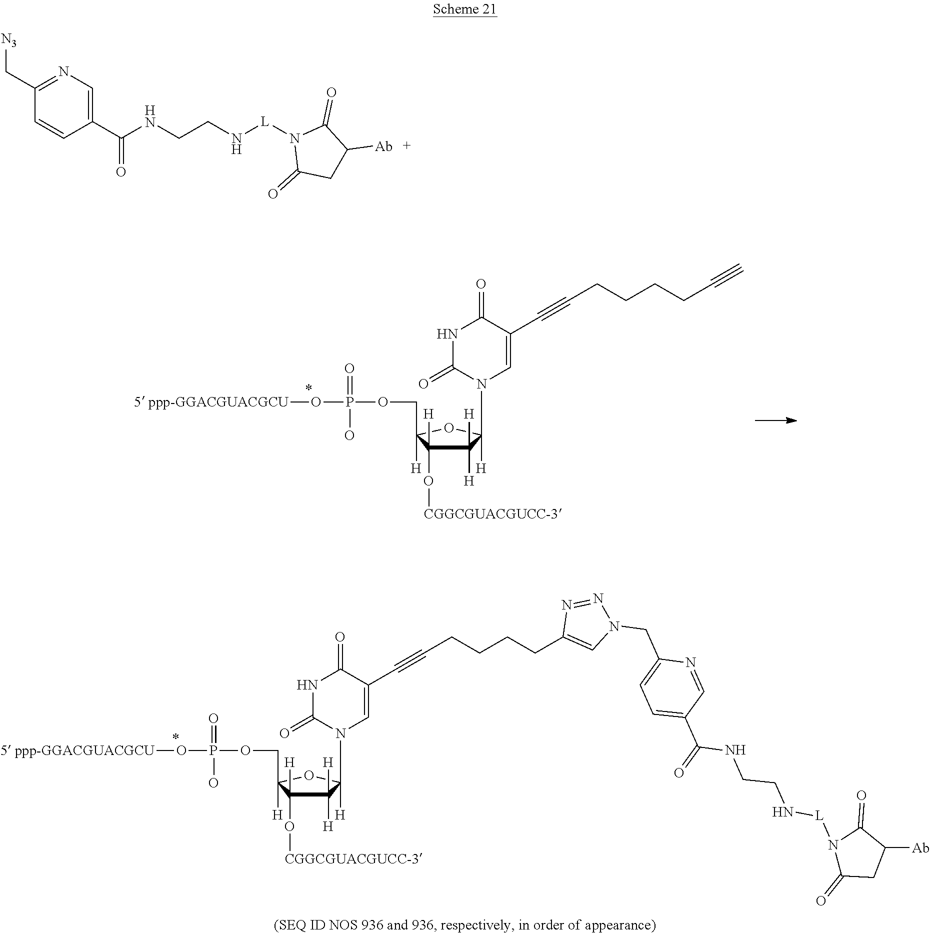

where the azide of the modified antibody (Ab) reacts via click chemistry with a pendant alkyne group off of the RNA hairpin to form a corresponding triazole group. L and Ab are as defined herein for compounds of Formula (V).

A general reaction scheme for the formation of certain immunostimulatory conjugates of Formula (V) is shown in Scheme 22 below:

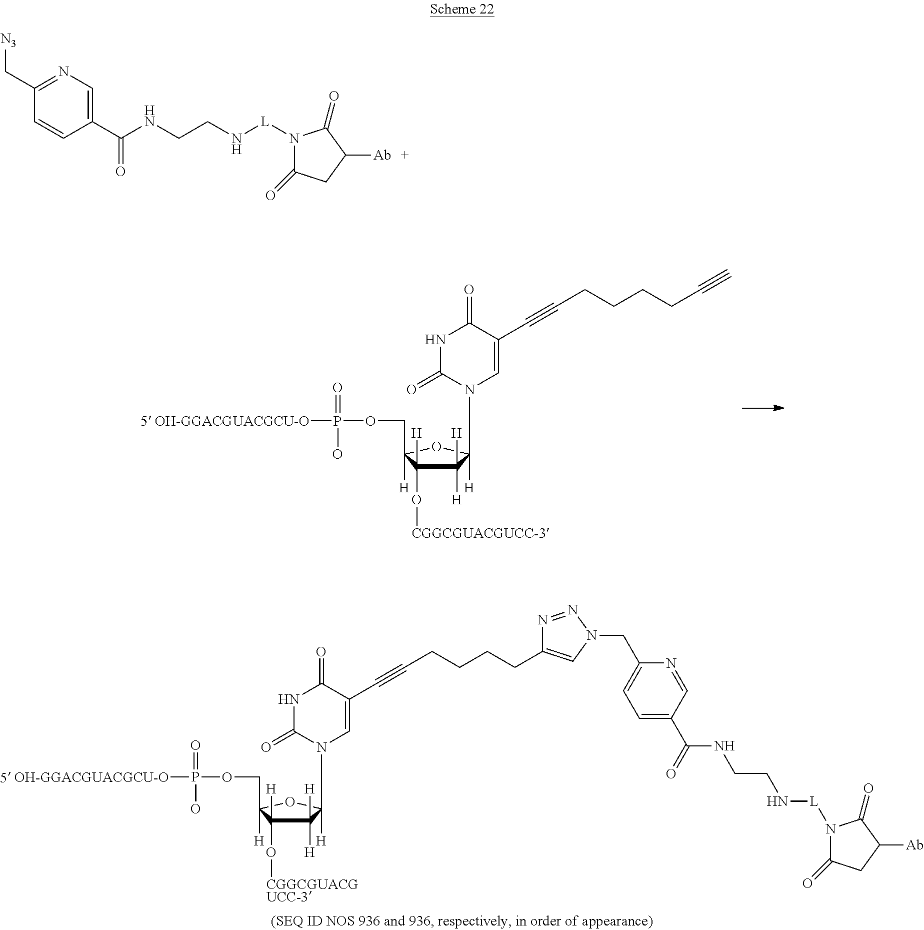

where the azide of the modified antibody (Ab) reacts via click chemistry with a pendant alkyne group off of the RNA hairpin to form a corresponding triazole group. L and Ab are as defined herein for compounds of Formula (V).

### Therapeutic Uses and Methods of Treatment

The anti-DC-SIGN antibodies, fragments thereof (e.g. antigen binding fragments thereof), antibody conjugates, and fusion proteins disclosed herein are useful in a variety of applications including, but not limited to, treatment of cancer. In certain embodiments, the antibodies, fragments thereof, antibody conjugates, or fusion proteins provided herein are useful for inhibiting tumor growth, reducing tumor volume, inducing differentiation, and/or reducing the tumorigenicity of a tumor. The methods of use can be in vitro, ex vivo, or in vivo methods.

In some embodiments, provided herein are methods of treating, preventing, or ameliorating a disease, e.g., a cancer, in a subject in need thereof, e.g., a human patient, by administering to the subject any of the antibodies, antibody fragments, antibody conjugates, or fusion proteins described herein. Also provided is use of the antibodies, antibody fragments, antibody conjugates, or fusion proteins of the invention to treat or prevent disease in a subject, e.g., a human patient. Additionally provided is use of antibody conjugates in treatment or prevention of disease in a subject. In some embodiments provided are antibody conjugates for use in manufacture of a medicament for treatment or prevention of disease in a subject. In certain embodiments, the disease treated with antibody conjugates is a cancer.

In one aspect, the antibodies, antibody fragments, antibody conjugates, or fusion proteins described herein can be used to treat a solid tumor. Examples of solid tumors include malignancies, e.g., sarcomas, adenocarcinomas, blastomas, and carcinomas, of the various organ systems, such as those affecting liver, lung, breast, lymphoid, biliarintestinal (e.g., colon), genitourinary tract (e.g., renal, urothelial cells), prostate and pharynx. Adenocarcinomas include malignancies such as most colon cancers, rectal cancer, renal-cell carcinoma, liver cancer, small cell lung cancer, non-small cell carcinoma of the lung, cancer of the small intestine and cancer of the esophagus. In one embodiment, the cancer is a melanoma, e.g., an advanced stage melanoma. Examples of other cancers that can be treated include bone cancer, pancreatic cancer, skin cancer, cancer of the head or neck, cutaneous or intraocular malignant melanoma, uterine cancer, ovarian cancer, rectal cancer, colorectal cancer, cancer of the anal region, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, environmentally induced cancers including those induced by asbestos, and combinations of said cancers.

In another aspect, the antibodies, antibody fragments, antibody conjugates, or fusion proteins described herein can be used to treat a hematological cancer. Hematological cancers include leukemia, lymphoma, and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In some embodiments, the cancer is a hematologic cancer including but is not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with a tumor antigen expression includes, but not limited to, e.g., atypical and/or non-classical cancers, malignancies, precancerous conditions or proliferative diseases expressing a tumor antigen as described herein. Metastatic lesions of the aforementioned cancers can also be treated or prevented using the methods and compositions of the invention.

In another aspect, the fusion proteins disclosed herein are useful in a variety of applications including, but not limited to, treatment of autoimmune disease. Examples of autoimmune diseases in which specific antigens have been identified as potentially pathogenically significant include multiple sclerosis (myelin basic protein), insulin-dependent diabetes mellitus (glutamic acid decarboxylase), insulin-resistant diabetes mellitus (insulin receptor), coeliac disease (gliadin), bullous pemphigoid (collagen type XVII), auto-immune haemolytic anaemia (Rh protein), auto-immune thrombocytopenia (GpIIb/IIIa), myaesthenia gravis (acetylcholine receptor), Graves' disease (thyroid-stimulating hormone receptor), glomerulonephritis, such as Goodpasture's disease (alpha3(IV)NC1 collagen), and pernicious anaemia (intrinsic factor).

Other autoimmune disease that may be treated with the fusion proteins disclosed herein include arthritis (e.g. rheumatoid arthritis), inflammatory bowel disease, gastritis, pernicious anaemia, thyroiditis, insulitis, diabetes, sialoadenitis, adrenalitis, autoimmune orchitis/oophoritis, glomerulonephritis, chronic obstructive pulmonary disease and experimental autoimmune encephalitis and multiple sclerosis.

In some embodiments, the fusion proteins disclosed herein may be used to treat a disease caused by an exogenous antigen which stimulates a response which also causes damage to host tissues. For example, acute rheumatic fever is caused by an antibody response to a Streptococcal antigen which cross-reacts with a cardiac muscle cell antigen. Thus these antigens, or particular fragments or epitopes thereof may be suitable antigens for use in the present invention. In some embodiments, the fusion proteins disclosed herein may be used to treat atherosclerosis, graft-versus-host disease, etc.

Method of administration of antibodies, antibody fragments, antibody conjugates, or fusion proteins include, but are not limited to, parenteral (e.g., intravenous) administration, e.g., injection as a bolus or continuous infusion over a period of time, oral administration, intramuscular administration, intratumoral administration, intramuscular administration, intraperitoneal administration, intracerobrospinal administration, subcutaneous administration, intra-articular administration, intrasynovial administration, injection to lymph nodes, or intrathecal administration.

For treatment of disease, appropriate dosage of antibodies, antibody fragments, antibody conjugates, or fusion proteins of the present invention depends on various factors, such as the type of disease to be treated, the severity and course of the disease, the responsiveness of the disease, previous therapy, patient's clinical history, and so on. Antibody conjugates can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of a particular antibody conjugate. In some embodiments, dosage is from 0.01 mg to 20 mg (e.g., 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg) per kg of body weight, and can be given once or more daily, weekly, monthly or yearly. In certain embodiments, the antibody conjugate of the present invention is given once every two weeks or once every three weeks. In certain embodiments, the antibody conjugate of the present invention is given only once. The treating physician can estimate repetition rates for dosing based on measured residence times and concentrations of the drug in bodily fluids or tissues.

### Combination Therapy

In certain instances, the anti-DC-SIGN antibodies, antibody fragments, antibody conjugates, or fusion proteins of the present invention can be combined with other therapeutic agents, such as other anti-cancer agents, anti-allergic agents, anti-nausea agents (or anti-emetics), pain relievers, cytoprotective agents, and combinations thereof.

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In one embodiment, the present invention provides a method of treating cancer by administering to a subject in need thereof the anti-DC-SIGN antibodies, antibody fragments, antibody conjugates, or fusion proteins of the present invention in combination with one or more anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In one embodiment, the present invention provides a method of treating cancer by administering to a subject in need thereof the anti-DC-SIGN antibodies, antibody fragments, antibody conjugates, or fusion proteins of the present invention in combination with one or more tyrosine kinase inhibitors, including but not limited to, EGFR inhibitors, Her3 inhibitors, IGFR inhibitors, and Met inhibitors.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif®); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PK1166, CAS187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl)methoxy]phenyl]amino]-3-cyano-7-ethoxy quinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and BI836845. See e.g., Yee, JNCI, 104; 975 (2012) for review.

In another embodiment, the present invention provides a method of treating cancer by administering to a subject in need thereof the anti-DC-SIGN antibodies, antibody fragments, antibody conjugates, or fusion proteins of the present invention in combination with one or more proliferation signaling pathway inhibitors, including but not limited to, MEK inhibitors, BRAF inhibitors, PI3K/Akt inhibitors, SHP2 inhibitors, and also mTOR inhibitors, and CDK inhibitors.

For example, mitogen-activated protein kinase (MEK) inhibitors include but are not limited to, XL-518 (also known as GDC-0973, Cas No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®), or Ipilimumab (or MDX-010, MDX-101, or Yervoy).

Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)—N1-(4-methyl-5-(2-(1,1,1-trifluoro-2-methylpropan-2-yl)pyridin-4-yl)thiazol-2-yl)pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDC0032, RG7604, or Taselisib).

mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1) (SEQ ID NO: 938).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, the present invention provides a method of treating cancer by administering to a subject in need thereof the anti-DC-SIGN antibodies, antibody fragments, antibody conjugates, or fusion proteins of the present invention in combination with one or more pro-apoptotics, including but not limited to, IAP inhibitors, BCL2 inhibitors, MCl1 inhibitors, TRAIL agents, CHK inhibitors.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL (SEQ ID NO: 939)), and CBP501 ((d-Bpa)sws(d-Phe-F5)(d-Cha)rrrqrr).

In a further embodiment, the present invention provides a method of treating cancer by administering to a subject in need thereof the anti-DC-SIGN antibodies, antibody fragments, antibody conjugates, or fusion proteins of the present invention in combination with one or more immunomodulators (e.g., one or more of an activator of a costimulatory molecule or an inhibitor of an immune checkpoint molecule).

In certain embodiments, the immunomodulator is an activator of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### GITR Agonists

In certain embodiments, the agonist of the costimulatory molecule is a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 2 (e.g., from the heavy and light chain variable region sequences of MAB7 disclosed in Table 2), or encoded by a nucleotide sequence shown in Table 2. In some embodiments, the CDRs are according to the Kabat definition (e.g., as set out in Table 2). In some embodiments, the CDRs are according to the Chothia definition (e.g., as set out in Table 2). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 2, or encoded by a nucleotide sequence shown in Table 2.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 2.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904. In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85%, 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85%, 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 2
Amino acid and nucleotide sequences
of exemplary anti-GITR antibody molecule

| | | |
|---|---|---|
| MAB7 | | |
| SEQ ID NO: 901 | VH | EVQLVESGGGLVQSGGSLRLSCAASGFSLSSYGVDW |
| | | VRQAPGKGLEWVGVIWGGGGTYYASSLMGRFTISRD |
| | | NSKNTLYLQMNSLRAEDTAVYYCARHAYGHDGGFAM |
| | | DYWGQGTLVTVSS |
| SEQ ID NO: 902 | VL | EIVMTQSPATLSVSPGERATLSCRASESVSSNVAWYQ |
| | | QRPGQAPRLLIYGASNRATGIPARFSGSGSGTDFTLTI |
| | | SRLEPEDFAVYYCGQSYSYPFTFGQGTKLEIK |
| SEQ ID NO: 903 | Heavy | EVQLVESGGGLVQSGGSLRLSCAASGFSLSSYGVDW |
| | Chain | VRQAPGKGLEWVGVIWGGGGTYYASSLMGRFTISRD |
| | | NSKNTLYLQMNSLRAEDTAVYYCARHAYGHDGGFAM |
| | | DYWGQGTLVTVSSASTKGPSVFPLAPSSKSTSGGTA |
| | | ALGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPAPIEKTISKAKGQPREPQVYTLPPSREEMTKN |
| | | QVSLTCLVKGFYPSDIAVEWESNGQPENNYKTTPPVL |
| | | DSDGSFFLYSKLTVDKSRWQQGNVFSCSVMHEALHN |
| | | HYTQKSLSLSPGK |
| SEQ ID NO: 904 | Light | EIVMTQSPATLSVSPGERATLSCRASESVSSNVAWYQ |
| | Chain | QRPGQAPRLLIYGASNRATGIPARFSGSGSGTDFTLTI |
| | | SRLEPEDFAVYYCGQSYSYPFTFGQGTKLEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| SEQ ID NO: 905 | DNA | GAGGTGCAGCTGGTGGAATCTGGCGGCGGACTGG |
| | VH | TGCAGTCCGGCGGCTCTCTGAGACTGTCTTGCGCT |
| | | GCCTCCGGCTTCTCCCTGTCCTCTTACGGCGTGGA |
| | | CTGGGTGCGACAGGCCCCTGGCAAGGGCCTGGAA |
| | | TGGGTGGGAGTGATCTGGGGCGGAGGCGGCACCT |
| | | ACTACGCCTCTTCCCTGATGGGCCGGTTCACCATCT |
| | | CCCGGGACAACTCCAAGAACACCCTGTACCTGCAG |
| | | ATGAACTCCCTGCGGGCCGAGGACACCGCCGTGTA |
| | | CTACTGCGCCAGACACGCCTACGGCCACGACGGC |
| | | GGCTTCGCCATGGATTATTGGGGCCAGGGCACCCT |
| | | GGTGACAGTGTCCTCC |
| SEQ ID NO: 906 | DNA | GAGATCGTGATGACCCAGTCCCCCGCCACCCTGTC |
| | VL | TGTGTCTCCCGGCGAGAGAGCCACCCTGAGCTGCA |
| | | GAGCCTCCGAGTCCGTGTCCTCCAACGTGGCCTGG |
| | | TATCAGCAGAGACCTGGTCAGGCCCCTCGGCTGCT |
| | | GATCTACGGCGCCTCTAACCGGGCCACCGGCATCC |
| | | CTGCCAGATTCTCCGGCTCCGGCAGCGGCACCGAC |
| | | TTCACCCTGACCATCTCCCGGCTGGAACCCGAGGA |
| | | CTTCGCCGTGTACTACTGCGGCCAGTCCTACTCATA |
| | | CCCCTTCACCTTCGGCCAGGGCACCAAGCTGGAAA |
| | | TCAAG |
| SEQ ID NO: 907 | DNA | GAGGTGCAGCTGGTGGAATCTGGCGGCGGACTGG |
| | Heavy | TGCAGTCCGGCGGCTCTCTGAGACTGTCTTGCGCT |
| | Chain | GCCTCCGGCTTCTCCCTGTCCTCTTACGGCGTGGA |
| | | CTGGGTGCGACAGGCCCCTGGCAAGGGCCTGGAA |
| | | TGGGTGGGAGTGATCTGGGGCGGAGGCGGCACCT |
| | | ACTACGCCTCTTCCCTGATGGGCCGGTTCACCATCT |
| | | CCCGGGACAACTCCAAGAACACCCTGTACCTGCAG |
| | | ATGAACTCCCTGCGGGCCGAGGACACCGCCGTGTA |
| | | CTACTGCGCCAGACACGCCTACGGCCACGACGGC |
| | | GGCTTCGCCATGGATTATTGGGGCCAGGGCACCCT |
| | | GGTGACAGTGTCCTCCGCTAGCACCAAGGGCCCAA |
| | | GTGTGTTTCCCCTGGCCCCCAGCAGCAAGTCTACTT |
| | | CCGGCGGAACTGCTGCCCTGGGTTGCCTGGTGAAG |
| | | GACTACTTCCCCGAGCCCGTGACAGTGTCCTGGAA |
| | | CTCTGGGGCTCTGACTTCCGGCGTGCACACCTTCC |
| | | CCGCCGTGCTGCAGAGCAGCGGCCTGTACAGCCT |
| | | GAGCAGCGTGGTGACAGTGCCCTCCAGCTCTCTGG |
| | | GAACCCAGACCTATATCTGCAACGTGAACCACAAGC |
| | | CCAGCAACACCAAGGTGGACAAGAGAGTGGAGCCC |
| | | AAGAGCTGCGACAAGACCCACACCTGCCCCCCCTG |
| | | CCCAGCTCCAGAACTGCTGGGAGGGCCTTCCGTGT |
| | | TCCTGTTCCCCCCCAAGCCCAAGGACACCCTGATG |
| | | ATCAGCAGGACCCCCGAGGTGACCTGCGTGGTGGT |
| | | GGACGTGTCCCACGAGGACCCAGAGGTGAAGTTCA |
| | | ACTGGTACGTGGACGGCGTGGAGGTGCACAACGC |
| | | CAAGACCAAGCCCAGAGAGGAGCAGTACAACAGCA |
| | | CCTACAGGGTGGTGTCCGTGCTGACCGTGCTGCAC |
| | | CAGGACTGGCTGAACGGCAAAGAATACAAGTGCAA |
| | | AGTCTCCAACAAGGCCCTGCCAGCCCCAATCGAAA |
| | | AGACAATCAGCAAGGCCAAGGGCCAGCCACGGGA |
| | | GCCCCAGGTGTACACCCTGCCCCCCAGCCGGGAG |
| | | GAGATGACCAAGAACCAGGTGTCCCTGACCTGTCT |
| | | GGTGAAGGGCTTCTACCCCAGCGATATCGCCGTGG |
| | | AGTGGGAGAGCAACGGCCAGCCCGAGAACAACTAC |
| | | AAGACCACCCCCCCAGTGCTGGACAGCGACGGCA |
| | | GCTTCTTCCTGTACAGCAAGCTGACCGTGGACAAGT |
| | | CCAGGTGGCAGCAGGGCAACGTGTTCAGCTGCAGC |
| | | GTGATGCACGAGGCCCTGCACAACCACTACACCCA |
| | | GAAGTCCCTGAGCCTGAGCCCCGGCAAG |
| SEQ ID NO: 908 | DNA | GAGATCGTGATGACCCAGTCCCCCGCCACCCTGTC |
| | Light | TGTGTCTCCCGGCGAGAGAGCCACCCTGAGCTGCA |
| | Chain | GAGCCTCCGAGTCCGTGTCCTCCAACGTGGCCTGG |
| | | TATCAGCAGAGACCTGGTCAGGCCCCTCGGCTGCT |
| | | GATCTACGGCGCCTCTAACCGGGCCACCGGCATCC |
| | | CTGCCAGATTCTCCGGCTCCGGCAGCGGCACCGAC |
| | | TTCACCCTGACCATCTCCCGGCTGGAACCCGAGGA |
| | | CTTCGCCGTGTACTACTGCGGCCAGTCCTACTCATA |
| | | CCCCTTCACCTTCGGCCAGGGCACCAAGCTGGAAA |
| | | TCAAGCGTACGGTGGCCGCTCCCAGCGTGTTCATC |
| | | TTCCCCCCCAGCGACGAGCAGCTGAAGAGCGGCAC |
| | | CGCCAGCGTGGTGTGCCTGCTGAACAACTTCTACC |
| | | CCCGGGAGGCCAAGGTGCAGTGGAAGGTGGACAA |
| | | CGCCCTGCAGAGCGGCAACAGCCAGGAGAGCGTC |
| | | ACCGAGCAGGACAGCAAGGACTCCACCTACAGCCT |
| | | GAGCAGCACCCTGACCCTGAGCAAGGCCGACTACG |
| | | AGAAGCATAAGGTGTACGCCTGCGAGGTGACCCAC |
| | | CAGGGCCTGTCCAGCCCCGTGACCAAGAGCTTCAA |
| | | CAGGGGCGAGTGC |
| SEQ ID NO: 909 | HCDR1 | SYGVD |
| (KABAT) | | |
| SEQ ID NO: 910 | HCDR1 | GFSLSSY |
| (CHOTHIA) | | |
| SEQ ID NO: 911 | HCDR2 | VIWGGGGTYYASSLMG |
| (KABAT) | | |
| SEQ ID NO: 912 | HCDR2 | WGGGG |
| (CHOTHIA) | | |
| SEQ ID NO: 913 | HCDR3 | HAYGHDGGFAMDY |
| (KABAT) | | |
| SEQ ID NO: 913 | HCDR3 | HAYGHDGGFAMDY |
| (CHOTHIA) | | |
| SEQ ID NO: 914 | LCDR1 | RASESVSSNVA |
| (KABAT) | | |
| SEQ ID NO: 915 | LCDR1 | SESVSSN |
| (CHOTHIA) | | |
| SEQ ID NO: 916 | LCDR2 | GASNRAT |
| (KABAT) | | |
| SEQ ID NO: 917 | LCDR2 | GAS |
| (CHOTHIA) | | |
| SEQ ID NO: 918 | LCDR3 | GQSYSYPFT |
| (KABAT) | | |
| SEQ ID NO: 919 | LCDR3 | SYSYPF |
| (CHOTHIA) | | |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 3.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MED11873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 3
Amino acid sequence
of other exemplary anti-GITR antibody molecules

| | | |
|---|---|---|
| BMS-986156 | | |
| SEQ ID NO: 920 | VH | QVQLVESGGGWQPGRSLRLSCAASGFTFSSY |
| | | GMHWVRQAPGKGLEWVAVIWYEGSNKYYADS |
| | | VKGRFTISRDNSKNTLYLQMNSLRAEDTAVY |
| | | YCARGGSMVRGDYYYGMDVWGQGTTVTVSS |
| SEQ ID NO: 921 | VL | AIQLTQSPSSLSASVGDRVTITCRASQGISS |
| | | ALAWYQQKPGKAPKLLIYDASSLESGVPSRF |
| | | SGSGSGTDFTLTISSLQPEDFATYYCQQFNS |
| | | YPYTFGQGTKLEIK |

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specificity, e.g., a second binding specificity to TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 and PD-L1. In yet another embodiment, the bispecific antibody molecule binds to PD-1 and PD-L2. In another embodiment, the bispecific antibody molecule binds to TIM-3 and LAG-3. Any combination of the aforesaid molecules can be made in a multispecific antibody molecule, e.g., a trispecific antibody that includes a first binding specificity to PD-1 or PD-1, and a second and third binding specificities to two or more of: TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule.

Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present invention. Any of the antibody molecules known in the art or disclosed herein can be used in the aforesaid combinations of inhibitors of checkpoint molecule.

### PD-1 Inhibitors

In some embodiments, the antibody conjugate of the present invention is administered in combination with a PD-1 inhibitor. In some embodiments, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 4 (e.g., from the heavy and light chain variable region sequences of BAP049-Clone-E or BAP049-Clone-B disclosed in Table 4), or encoded by a nucleotide sequence shown in Table 4. In some embodiments, the CDRs are according to the Kabat definition (e.g., as set out in Table 4). In some embodiments, the CDRs are according to the Chothia definition (e.g., as set out in Table 4). In some embodiments, the CDRs are according to the combined CDR definitions of both Kabat and Chothia (e.g., as set out in Table 4). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 4, or encoded by a nucleotide sequence shown in Table 4.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 4.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 4.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 4
Amino acid and nucleotide sequences
of exemplary anti-PD-1 antibody molecules

| | | |
|---|---|---|
| BAP049-Clone-B HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQ |
| | | ATGQGLEWMGNIYPGTGGSNFDEKFKNRVTITADKSTSTA |
| | | YMELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAA |
| | VH | GCCCGGCGAGTCACTGAGAATTAGCTGTAAAGGTTCAG |
| | | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQ |
| | chain | ATGQGLEWMGNIYPGTGGSNFDEKFKNRVTITADKSTSTA |
| | | YMELSSLRSEDTAVYYCTRWTTGTGAYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 509 | DNA | GAGGTGCAGCTGGTGCAGTCAGGCGCCGAAGTGAAGAA |
| | heavy | GCCCGGCGAGTCACTGAGAATTAGCTGTAAAGGTTCAG |
| | chain | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGCGCTAGCACTAAGGGCCCGTCCGTGTTCCCCCT |
| | | GGCACCTTGTAGCCGGAGCACTAGCGAATCCACCGCTG |
| | | CCCTCGGCTGCCTGGTCAAGGATTACTTCCCGGAGCCC |
| | | GTGACCGTGTCCTGGAACAGCGGAGCCCTGACCTCCGG |
| | | AGTGCACACCTTCCCCGCTGTGCTGCAGAGCTCCGGGC |
| | | TGTACTCGCTGTCGTCGGTGGTCACGGTGCCTTCATCTA |
| | | GCCTGGGTACCAAGACCTACACTTGCAACGTGGACCAC |
| | | AAGCCTTCCAACACTAAGGTGGACAAGCGCGTCGAATC |
| | | GAAGTACGGCCCACCGTGCCCGCCTTGTCCCGCGCCG |
| | | GAGTTCCTCGGCGGTCCCTCGGTCTTTCTGTTCCCACC |
| | | GAAGCCCAAGGACACTTTGATGATTTCCCGCACCCCTGA |
| | | AGTGACATGCGTGGTCGTGGACGTGTCACAGGAAGATC |
| | | CGGAGGTGCAGTTCAATTGGTACGTGGATGGCGTCGAG |
| | | GTGCACAACGCCAAAACCAAGCCGAGGGAGGAGCAGTT |
| | | CAACTCCACTTACCGCGTCGTGTCCGTGCTGACGGTGC |
| | | TGCATCAGGACTGGCTGAACGGGAAGGAGTACAAGTGC |
| | | AAAGTGTCCAACAAGGGACTTCCTAGCTCAATCGAAAAG |
| | | ACCATCTCGAAAGCCAAGGGACAGCCCCGGGAACCCCA |
| | | AGTGTATACCCTGCCACCGAGCCAGGAAGAAATGACTAA |
| | | GAACCAAGTCTCATTGACTTGCCTTGTGAAGGGCTTCTA |
| | | CCCATCGGATATCGCCGTGGAATGGGAGTCCAACGGCC |
| | | AGCCGGAAAACAACTACAAGACCACCCCTCCGGTGCTG |
| | | GACTCAGACGGATCCTTCTTCCTCTACTCGCGGCTGACC |
| | | GTGGATAAGAGCAGATGGCAGGAGGGAAATGTGTTCAG |
| | | CTGTTCTGTGATGCATGAAGCCCTGCACAACCACTACAC |
| | | TCAGAAGTCCCTGTCCCTCTCCCTGGGA |
| BAP049-Clone-B LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTW |
| | | YQQKPGKAPKLLIYWASTRESGVPSRFSGSGSGTDFTFTIS |
| | | SLQPEDIATYYCQNDYSYPYTFGQGTKVEIK |
| SEQ ID NO: 517 | DNA | GAGATCGTCCTGACTCAGTCACCCGCTACCCTGAGCCT |
| | VL | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTAAAGCCCCTAAG |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGCAGCCCGAGGATATCGC |
| | | TACCTACTACTGTCAGAACGACTATAGCTACCCCTACAC |
| | | CTTCGGTCAAGGCACTAAGGTCGAGATTAAG |
| SEQ ID NO: 518 | Light | EIVLTQSPATLSLSPGERATLSCKSSQSLLDSGNQKNFLTW |
| | chain | YQQKPGKAPKLLIYWASTRESGVPSRFSGSGSGTDFTFTIS |
| | | SLQPEDIATYYCQNDYSYPYTFGQGTKVEIKRTVAAPSVFIF |
| | | PPSDEQLKSGTAS\NCLLNNFYPREAKVQWKVDNALQSG |
| | | NSQESVTEQDSKDSTYSLSSTLTLSKADYEKHKVYACEVT |
| | | HQGLSSPVTKSFNRGEC |
| SEQ ID NO: 519 | DNA | GAGATCGTCCTGACTCAGTCACCCGCTACCCTGAGCCT |
| | light | GAGCCCTGGCGAGCGGGCTACACTGAGCTGTAAATCTA |
| | chain | GTCAGTCACTGCTGGATAGCGGTAATCAGAAGAACTTCC |
| | | TGACCTGGTATCAGCAGAAGCCCGGTAAAGCCCCTAAG |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGCAGCCCGAGGATATCGC |
| | | TACCTACTACTGTCAGAACGACTATAGCTACCCCTACAC |
| | | CTTCGGTCAAGGCACTAAGGTCGAGATTAAGCGTACGG |
| | | TGGCCGCTCCCAGCGTGTTCATCTTCCCCCCCAGCGAC |
| | | GAGCAGCTGAAGAGCGGCACCGCCAGCGTGGTGTGCC |
| | | TGCTGAACAACTTCTACCCCCGGGAGGCCAAGGTGCAG |
| | | TGGAAGGTGGACAACGCCCTGCAGAGCGGCAACAGCCA |
| | | GGAGAGCGTCACCGAGCAGGACAGCAAGGACTCCACCT |
| | | ACAGCCTGAGCAGCACCCTGACCCTGAGCAAGGCCGAC |
| | | TACGAGAAGCATAAGGTGTACGCCTGCGAGGTGACCCA |
| | | CCAGGGCCTGTCCAGCCCCGTGACCAAGAGCTTCAACA |
| | | GGGGCGAGTGC |
| BAP049-Clone-E HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQ |
| | | ATGQGLEWMGNIYPGTGGSNFDEKFKNRVTITADKSTSTA |
| | | YMELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAA |
| | VH | GCCCGGCGAGTCACTGAGAATTAGCTGTAAAGGTTCAG |
| | | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQ |
| | chain | ATGQGLEWMGNIYPGTGGSNFDEKFKNRVTITADKSTSTA |
| | | YMELSSLRSEDTAVYYCTRWTTGTGAYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 509 | DNA | GAGGTGCAGCTGGTGCAGTCAGGCGCCGAAGTGAAGAA |
| | heavy | GCCCGGCGAGTCACTGAGAATTAGCTGTAAAGGTTCAG |
| | chain | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGCGCTAGCACTAAGGGCCCGTCCGTGTTCCCCCT |
| | | GGCACCTTGTAGCCGGAGCACTAGCGAATCCACCGCTG |
| | | CCCTCGGCTGCCTGGTCAAGGATTACTTCCCGGAGCCC |
| | | GTGACCGTGTCCTGGAACAGCGGAGCCCTGACCTCCGG |
| | | AGTGCACACCTTCCCCGCTGTGCTGCAGAGCTCCGGGC |
| | | TGTACTCGCTGTCGTCGGTGGTCACGGTGCCTTCATCTA |
| | | GCCTGGGTACCAAGACCTACACTTGCAACGTGGACCAC |
| | | AAGCCTTCCAACACTAAGGTGGACAAGCGCGTCGAATC |
| | | GAAGTACGGCCCACCGTGCCCGCCTTGTCCCGCGCCG |
| | | GAGTTCCTCGGCGGTCCCTCGGTCTTTCTGTTCCCACC |
| | | GAAGCCCAAGGACACTTTGATGATTTCCCGCACCCCTGA |
| | | AGTGACATGCGTGGTCGTGGACGTGTCACAGGAAGATC |
| | | CGGAGGTGCAGTTCAATTGGTACGTGGATGGCGTCGAG |
| | | GTGCACAACGCCAAAACCAAGCCGAGGGAGGAGCAGTT |
| | | CAACTCCACTTACCGCGTCGTGTCCGTGCTGACGGTGC |
| | | TGCATCAGGACTGGCTGAACGGGAAGGAGTACAAGTGC |
| | | AAAGTGTCCAACAAGGGACTTCCTAGCTCAATCGAAAAG |
| | | ACCATCTCGAAAGCCAAGGGACAGCCCCGGGAACCCCA |
| | | AGTGTATACCCTGCCACCGAGCCAGGAAGAAATGACTAA |
| | | GAACCAAGTCTCATTGACTTGCCTTGTGAAGGGCTTCTA |
| | | CCCATCGGATATCGCCGTGGAATGGGAGTCCAACGGCC |
| | | AGCCGGAAAACAACTACAAGACCACCCCTCCGGTGCTG |
| | | GACTCAGACGGATCCTTCTTCCTCTACTCGCGGCTGACC |
| | | GTGGATAAGAGCAGATGGCAGGAGGGAAATGTGTTCAG |
| | | CTGTTCTGTGATGCATGAAGCCCTGCACAACCACTACAC |
| | | TCAGAAGTCCCTGTCCCTCTCCCTGGGA |
| BAP049-Clone-E LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTW |
| | | YQQKPGQAPRLLIYWASTRESGVPSRFSGSGSGTDFTFTI |
| | | SSLEAEDAATYYCQNDYSYPYTFGQGTKVEIK |
| SEQ ID NO: 521 | DNA | GAGATCGTCCTGACTCAGTCACCCGCTACCCTGAGCCT |
| | VL | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTCAAGCCCCTAGA |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGGAAGCCGAGGACGCCG |
| | | CTACCTACTACTGTCAGAACGACTATAGCTACCCCTACA |
| | | CCTTCGGTCAAGGCACTAAGGTCGAGATTAAG |
| SEQ ID NO: 522 | Light | EIVLTQSPATLSLSPGERATLSCKSSQSLLDSGNQKNFLTW |
| | chain | YQQKPGQAPRLLIYWASTRESGVPSRFSGSGSGTDFTFTI |
| | | SSLEAEDAATYYCQNDYSYPYTFGQGTKVEIKRTVAAPSVF |
| | | IFPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| SEQ ID NO: 523 | DNA | GAGATCGTCCTGACTCAGTCACCCGCTACCCTGAGCCT |
| | light | GAGCCCTGGCGAGCGGGCTACACTGAGCTGTAAATCTA |
| | chain | GTCAGTCACTGCTGGATAGCGGTAATCAGAAGAACTTCC |
| | | TGACCTGGTATCAGCAGAAGCCCGGTCAAGCCCCTAGA |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGGAAGCCGAGGACGCCG |
| | | CTACCTACTACTGTCAGAACGACTATAGCTACCCCTACA |
| | | CCTTCGGTCAAGGCACTAAGGTCGAGATTAAGCGTACG |
| | | GTGGCCGCTCCCAGCGTGTTCATCTTCCCCCCCAGCGA |
| | | CGAGCAGCTGAAGAGCGGCACCGCCAGCGTGGTGTGC |
| | | CTGCTGAACAACTTCTACCCCCGGGAGGCCAAGGTGCA |
| | | GTGGAAGGTGGACAACGCCCTGCAGAGCGGCAACAGC |
| | | CAGGAGAGCGTCACCGAGCAGGACAGCAAGGACTCCAC |
| | | CTACAGCCTGAGCAGCACCCTGACCCTGAGCAAGGCCG |
| | | ACTACGAGAAGCATAAGGTGTACGCCTGCGAGGTGACC |
| | | CACCAGGGCCTGTCCAGCCCCGTGACCAAGAGCTTCAA |
| | | CAGGGGCGAGTGC |
| BAP049-Clone-B HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGA |
| (Kabat) | | GAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGA |
| (Kabat) | | GAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

### Other Exemplary PD-1 Inhibitors

In some embodiments, the anti-PD-1 antibody is Nivolumab (CAS Registry Number: 946414-94-4). Alternative names for Nivolumab include MDX-1106, MDX-1106-04, ONO-4538, BMS-936558 or OPDIVO®. Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and PCT Publication No. WO2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 5.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, PCT Publication No. WO2009/114335, and U.S. Pat. No. 8,354,509, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 5.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in PCT Publication No. WO2009/101611, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 5.

Other anti-PD1 antibodies are disclosed in U.S. Pat. No. 8,609,089, US Publication No. 2010028330, and/or US Publication No. 20120114649, incorporated by reference in their entirety. Other anti-PD1 antibodies include AMP 514 (Amplimmune).

In one embodiment, the anti-PD-1 antibody molecule is MED10680 (Medimmune), also known as AMP-514. MED10680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg (Amplimmune), e.g., disclosed in WO 2010/027827 and WO 2011/066342, incorporated by reference in their entirety).

TABLE 5
Amino acid sequences of other exemplary anti-PD-1 antibody molecules

| | | |
|---|---|---|
| Nivolumab | | |
| SEQ ID NO: 535 | Heavy | QVQLVESGGGVVQPGRSLRLDCKASGITFSNSGMHWVRQAPG |
| | chain | KGLEWVAVIWYDGSKRYYADSVKGRFTISRDNSKNTLFLQMNSL |
| | | RAEDTAVYYCATNDDYWGQGTLVTVSSASTKGPSVFPLAPCSR |
| | | STSESTAALGCLVKDYFPEPVTVSWNSGALTSGVHTFPAVLQSS |
| | | GLYSLSSVVTVPSSSLGTKTYTCNVDHKPSNTKVDKRVESKYGP |
| | | PCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQ |
| | | DWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLS |
| | | LSLGK |
| SEQ ID NO: 536 | Light | EIVLTQSPATLSLSPGERATLSCRASQSVSSYLAWYQQKPGQAP |
| | chain | RLLIYDASNRATGIPARFSGSGSGTDFTLTISSLEPEDFAVYYCQ |
| | | QSSNWPRTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| Pembrolizumab | | |
| SEQ ID NO: 537 | Heavy | QVQLVQSGVEVKKPGASVKVSCKASGYTFTNYYMYWVRQAPG |
| | chain | QGLEWMGGINPSNGGTNFNEKFKNRVTLTTDSSTTTAYMELKSL |
| | | QFDDTAVYYCARRDYRFDMGFDYWGQGTTVTVSSASTKGPSV |
| | | FPLAPCSRSTSESTAALGCLVKDYFPEPVTVSWNSGALTSGVHT |
| | | FPAVLQSSGLYSLSSVVTVPSSSLGTKTYTCNVDHKPSNTKVDK |
| | | RVESKYGPPCPPCPAPEFLGGPSVFLFPPKPKDTLMISRTPEVT |
| | | CVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPEN |
| | | NYKTTPPVLDSDGSFFLYSRLTVDKSRWQEGNVFSCSVMHEAL |
| | | HNHYTQKSLSLSLGK |
| SEQ ID NO: 538 | Light | EIVLTQSPATLSLSPGERATLSCRASKGVSTSGYSYLHWYQQKP |
| | chain | GQAPRLLIYLASYLESGVPARFSGSGSGTDFTLTISSLEPEDFAV |
| | | YYCQHSRDLPLTFGGGTKVEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| Pidilizumab | | |
| SEQ ID NO: 539 | Heavy | QVQLVQSGSELKKPGASVKISCKASGYTFTNYGMNWVRQAPGQ |
| | chain | GLQWMGWINTDSGESTYAEEFKGRFVFSLDTSVNTAYLQITSLT |
| | | AEDTGMYFCVRVGYDALDYWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKRVEPK |
| | | SCDKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| SEQ ID NO: 540 | Light | EIVLTQSPSSLSASVGDRVTITCSARSSVSYMHWFQQKPGKAPK |
| | chain | LWIYRTSNLASGVPSRFSGSGSGTSYCLTINSLQPEDFATYYCQ |
| | | QRSSFPLTFGGGTKLEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |

### PD-L1 Inhibitors

In certain embodiments, the inhibitor of an immune checkpoint molecule is an inhibitor of PD-L1. In some embodiments, the antibody conjugate of the present invention is administered in combination with a PD-L1 inhibitor. In some embodiments, the PD-L1 inhibitor is selected from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

### Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 6 (e.g., from the heavy and light chain variable region sequences of BAP058-Clone O or BAP058-Clone N disclosed in Table 6), or encoded by a nucleotide sequence shown in Table 6. In some embodiments, the CDRs are according to the Kabat definition (e.g., as set out in Table 6). In some embodiments, the CDRs are according to the Chothia definition (e.g., as set out in Table 6). In some embodiments, the CDRs are according to the combined CDR definitions of both Kabat and Chothia (e.g., as set out in Table 6). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 6, or encoded by a nucleotide sequence shown in Table 6.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 6.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 6.

In one embodiment, the anti-PD-L1 antibody molecule comprises a VH comprising the amino acid sequence of SEQ ID NO: 606, or an amino acid sequence at least 85%, 90%, 95%, or 99% identical or higher to SEQ ID NO: 606. In one embodiment, the anti-PD-L1 antibody molecule comprises a VL comprising the amino acid sequence of SEQ ID NO: 616, or an amino acid sequence at least 85%, 90%, 95%, or 99% identical or higher to SEQ ID NO: 616. In one embodiment, the anti-PD-L1 antibody molecule comprises a VH comprising the amino acid sequence of SEQ ID NO: 620, or an amino acid sequence at least 85%, 90%, 95%, or 99% identical or higher to SEQ ID NO: 620. In one embodiment, the anti-PD-L1 antibody molecule comprises a VL comprising the amino acid sequence of SEQ ID NO: 624, or an amino acid sequence at least 85%, 90%, 95%, or 99% identical or higher to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence at least 85%, 90%, 95%, or 99% identical or higher to SEQ ID NO: 607. In one embodiment, the antibody molecule comprises a VL encoded by the nucleotide sequence of SEQ ID NO: 617, or a nucleotide sequence at least 85%, 90%, 95%, or 99% identical or higher to SEQ ID NO: 617. In one embodiment, the antibody molecule comprises a VH encoded by the nucleotide sequence of SEQ ID NO: 621, or a nucleotide sequence at least 85%, 90%, 95%, or 99% identical or higher to SEQ ID NO: 621. In one embodiment, the antibody molecule comprises a VL encoded by the nucleotide sequence of SEQ ID NO: 625, or a nucleotide sequence at least 85%, 90%, 95%, or 99% identical or higher to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence at least 85%, 90%, 95%, or 99% identical or higher to SEQ ID NO: 608. In one embodiment, the anti-PD-L1 antibody molecule comprises a light chain comprising the amino acid sequence of SEQ ID NO: 618, or an amino acid sequence at least 85%, 90%, 95%, or 99% identical or higher to SEQ ID NO: 618. In one embodiment, the anti-PD-L1 antibody molecule comprises a heavy chain comprising the amino acid sequence of SEQ ID NO: 622, or an amino acid sequence at least 85%, 90%, 95%, or 99% identical or higher to SEQ ID NO: 622. In one embodiment, the anti-PD-L1 antibody molecule comprises a light chain comprising the amino acid sequence of SEQ ID NO: 626, or an amino acid sequence at least 85%, 90%, 95%, or 99% identical or higher to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence at least 85%, 90%, 95%, or 99% identical or higher to SEQ ID NO: 615. In one embodiment, the antibody molecule comprises a light chain encoded by the nucleotide sequence of SEQ ID NO: 619, or a nucleotide sequence at least 85%, 90%, 95%, or 99% identical or higher to SEQ ID NO: 619. In one embodiment, the antibody molecule comprises a heavy chain encoded by the nucleotide sequence of SEQ ID NO: 623, or a nucleotide sequence at least 85%, 90%, 95%, or 99% identical or higher to SEQ ID NO: 623. In one embodiment, the antibody molecule comprises a light chain encoded by the nucleotide sequence of SEQ ID NO: 627, or a nucleotide sequence at least 85%, 90%, 95%, or 99% identical or higher to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627.

The antibody molecules described herein can be made by vectors, host cells, and methods described in US 2016/0108123, incorporated by reference in its entirety.

TABLE 6
Amino acid and nucleotide sequences of exemplary anti-PD-L1 antibody molecules

| | | |
|---|---|---|
| BAP058-Clone O HC | | |
| SEQ ID NO: 601 | HCDR1 | SYWMY |
| (Kabat) | | |
| SEQ ID NO: 602 | HCDR2 | RIDPNSGSTKYNEKFKN |
| (Kabat) | | |
| SEQ ID NO: 603 | HCDR3 | DYRKGLYAMDY |
| (Kabat) | | |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 606 | VH | EVQLVQSGAEVKKPGATVKISCKVSGYTFTSYWMYWV |
| | | RQARGQRLEWIGRIDPNSGSTKYNEKFKNRFTISRDNS |
| | | KNTLYLQMNSLRAEDTAVYYCARDYRKGLYAMDYWG |
| | | QGTTVTVSS |
| SEQ ID NO: 607 | DNA VH | GAAGTGCAGCTGGTGCAGTCAGGCGCCGAAGTGAA |
| | | GAAACCCGGCGCTACCGTGAAGATTAGCTGTAAAGT |
| | | CTCAGGCTACACCTTCACTAGCTACTGGATGTACTG |
| | | GGTCCGACAGGCTAGAGGGCAAAGACTGGAGTGGA |
| | | TCGGTAGAATCGACCCTAATAGCGGCTCTACTAAGTA |
| | | TAACGAGAAGTTTAAGAATAGGTTCACTATTAGTAGG |
| | | GATAACTCTAAGAACACCCTGTACCTGCAGATGAATA |
| | | GCCTGAGAGCCGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| SEQ ID NO: 608 | Heavy | EVQLVQSGAEVKKPGATVKISCKVSGYTFTSYWMYWV |
| | chain | RQARGQRLEWIGRIDPNSGSTKYNEKFKNRFTISRDNS |
| | | KNTLYLQMNSLRAEDTAVYYCARDYRKGLYAMDYWG |
| | | QGTTVTVSSASTKGPSVFPLAPCSRSTSESTAALGCLV |
| | | KDYFPEPVTVSWNSGALTSGVHTFPAVLQSSGLYSLSS |
| | | WTVPSSSLGTKTYTCNVDHKPSNTKVDKRVESKYGPP |
| | | CPPCPAPEFLGGPSVFLFPPKPKDTLMISRTPEVTCVV |
| | | VDVSQEDPEVQFNWYVDGVEVHNAKTKPREEQFNST |
| | | YRVVSVLTVLHQDWLNGKEYKCKVSNKGLPSSIEKTIS |
| | | KAKGQPREPQVYTLPPSQEEMTKNQVSLTCLVKGFYP |
| | | SDIAVEWESNGQPENNYKTTPPVLDSDGSFFLYSRLTV |
| | | DKSRWQEGNVFSCSVMHEALHNHYTQKSLSLSLG |
| SEQ ID NO: 615 | DNA | GAAGTGCAGCTGGTGCAGTCAGGCGCCGAAGTGAA |
| | heavy | GAAACCCGGCGCTACCGTGAAGATTAGCTGTAAAGT |
| | chain | CTCAGGCTACACCTTCACTAGCTACTGGATGTACTG |
| | | GGTCCGACAGGCTAGAGGGCAAAGACTGGAGTGGA |
| | | TCGGTAGAATCGACCCTAATAGCGGCTCTACTAAGTA |
| | | TAACGAGAAGTTTAAGAATAGGTTCACTATTAGTAGG |
| | | GATAACTCTAAGAACACCCTGTACCTGCAGATGAATA |
| | | GCCTGAGAGCCGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| | | GCTAGCACTAAGGGCCCGTCCGTGTTCCCCCTGGCA |
| | | CCTTGTAGCCGGAGCACTAGCGAATCCACCGCTGCC |
| | | CTCGGCTGCCTGGTCAAGGATTACTTCCCGGAGCCC |
| | | GTGACCGTGTCCTGGAACAGCGGAGCCCTGACCTC |
| | | CGGAGTGCACACCTTCCCCGCTGTGCTGCAGAGCTC |
| | | CGGGCTGTACTCGCTGTCGTCGGTGGTCACGGTGC |
| | | CTTCATCTAGCCTGGGTACCAAGACCTACACTTGCAA |
| | | CGTGGACCACAAGCCTTCCAACACTAAGGTGGACAA |
| | | GCGCGTCGAATCGAAGTACGGCCCACCGTGCCCGC |
| | | CTTGTCCCGCGCCGGAGTTCCTCGGCGGTCCCTCG |
| | | GTCTTTCTGTTCCCACCGAAGCCCAAGGACACTTTGA |
| | | TGATTTCCCGCACCCCTGAAGTGACATGCGTGGTCG |
| | | TGGACGTGTCACAGGAAGATCCGGAGGTGCAGTTCA |
| | | ATTGGTACGTGGATGGCGTCGAGGTGCACAACGCCA |
| | | AAACCAAGCCGAGGGAGGAGCAGTTCAACTCCACTT |
| | | ACCGCGTCGTGTCCGTGCTGACGGTGCTGCATCAG |
| | | GACTGGCTGAACGGGAAGGAGTACAAGTGCAAAGTG |
| | | TCCAACAAGGGACTTCCTAGCTCAATCGAAAAGACC |
| | | ATCTCGAAAGCCAAGGGACAGCCCCGGGAACCCCA |
| | | AGTGTATACCCTGCCACCGAGCCAGGAAGAAATGAC |
| | | TAAGAACCAAGTCTCATTGACTTGCCTTGTGAAGGGC |
| | | TTCTACCCATCGGATATCGCCGTGGAATGGGAGTCC |
| | | AACGGCCAGCCGGAAAACAACTACAAGACCACCCCT |
| | | CCGGTGCTGGACTCAGACGGATCCTTCTTCCTCTAC |
| | | TCGCGGCTGACCGTGGATAAGAGCAGATGGCAGGA |
| | | GGGAAATGTGTTCAGCTGTTCTGTGATGCATGAAGC |
| | | CCTGCACAACCACTACACTCAGAAGTCCCTGTCCCT |
| | | CTCCCTGGGA |
| BAP058-Clone O LC | | |
| SEQ ID NO: 609 | LCDR1 | KASQDVGTAVA |
| (Kabat) | | |
| SEQ ID NO: 610 | LCDR2 | WASTRHT |
| (Kabat) | | |
| SEQ ID NO: 611 | LCDR3 | QQYNSYPLT |
| (Kabat) | | |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 616 | VL | AIQLTQSPSSLSASVGDRVTITCKASQDVGTAVAWYLQ |
| | | KPGQSPQLLIYWASTRHTGVPSRFSGSGSGTDFTFTIS |
| | | SLEAEDAATYYCQQYNSYPLTFGQGTKVEIK |
| SEQ ID NO: 617 | DNA VL | GCTATTCAGCTGACTCAGTCACCTAGTAGCCTGAGC |
| | | GCTAGTGTGGGCGATAGAGTGACTATCACCTGTAAA |
| | | GCCTCTCAGGACGTGGGCACCGCCGTGGCCTGGTA |
| | | TCTGCAGAAGCCTGGTCAATCACCTCAGCTGCTGAT |
| | | CTACTGGGCCTCTACTAGACACACCGGCGTGCCCTC |
| | | TAGGTTTAGCGGTAGCGGTAGTGGCACCGACTTCAC |
| | | CTTCACTATCTCTTCACTGGAAGCCGAGGACGCCGC |
| | | TACCTACTACTGTCAGCAGTATAATAGCTACCCCCTG |
| | | ACCTTCGGTCAAGGCACTAAGGTCGAGATTAAG |
| SEQ ID NO: 618 | Light | AIQLTQSPSSLSASVGDRVTITCKASQDVGTAVAWYLQ |
| | chain | KPGQSPQLLIYWASTRHTGVPSRFSGSGSGTDFTFTIS |
| | | SLEAEDAATYYCQQYNSYPLTFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID NO: 619 | DNA light | GCTATTCAGCTGACTCAGTCACCTAGTAGCCTGAGC |
| | chain | GCTAGTGTGGGCGATAGAGTGACTATCACCTGTAAA |
| | | GCCTCTCAGGACGTGGGCACCGCCGTGGCCTGGTA |
| | | TCTGCAGAAGCCTGGTCAATCACCTCAGCTGCTGAT |
| | | CTACTGGGCCTCTACTAGACACACCGGCGTGCCCTC |
| | | TAGGTTTAGCGGTAGCGGTAGTGGCACCGACTTCAC |
| | | CTTCACTATCTCTTCACTGGAAGCCGAGGACGCCGC |
| | | TACCTACTACTGTCAGCAGTATAATAGCTACCCCCTG |
| | | ACCTTCGGTCAAGGCACTAAGGTCGAGATTAAGCGT |
| | | ACGGTGGCCGCTCCCAGCGTGTTCATCTTCCCCCCC |
| | | AGCGACGAGCAGCTGAAGAGCGGCACCGCCAGCGT |
| | | GGTGTGCCTGCTGAACAACTTCTACCCCCGGGAGGC |
| | | CAAGGTGCAGTGGAAGGTGGACAACGCCCTGCAGA |
| | | GCGGCAACAGCCAGGAGAGCGTCACCGAGCAGGAC |
| | | AGCAAGGACTCCACCTACAGCCTGAGCAGCACCCTG |
| | | ACCCTGAGCAAGGCCGACTACGAGAAGCATAAGGTG |
| | | TACGCCTGCGAGGTGACCCACCAGGGCCTGTCCAG |
| | | CCCCGTGACCAAGAGCTTCAACAGGGGCGAGTGC |
| BAP058-Clone N HC | | |
| SEQ ID NO: 601 | HCDR1 | SYWMY |
| (Kabat) | | |
| SEQ ID NO: 602 | HCDR2 | RIDPNSGSTKYNEKFKN |
| (Kabat) | | |
| SEQ ID NO: 603 | HCDR3 | DYRKGLYAMDY |
| (Kabat) | | |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 620 | VH | EVQLVQSGAEVKKPGATVKISCKVSGYTFTSYWMYWV |
| | | RQATGQGLEWMGRIDPNSGSTKYNEKFKNRVTITADK |
| | | STSTAYMELSSLRSEDTAVYYCARDYRKGLYAMDYWG |
| | | QGTTVTVSS |
| SEQ ID NO: 621 | DNA VH | GAAGTGCAGCTGGTGCAGTCAGGCGCCGAAGTGAA |
| | | GAAACCCGGCGCTACCGTGAAGATTAGCTGTAAAGT |
| | | CTCAGGCTACACCTTCACTAGCTACTGGATGTACTG |
| | | GGTCCGACAGGCTACCGGTCAAGGCCTGGAGTGGA |
| | | TGGGTAGAATCGACCCTAATAGCGGCTCTACTAAGT |
| | | ATAACGAGAAGTTTAAGAATAGAGTGACTATCACCGC |
| | | CGATAAGTCTACTAGCACCGCCTATATGGAACTGTCT |
| | | AGCCTGAGATCAGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| SEQ ID NO: 622 | Heavy | EVQLVQSGAEVKKPGATVKISCKVSGYTFTSYWMYWV |
| | chain | RQATGQGLEWMGRIDPNSGSTKYNEKFKNRVTITADK |
| | | STSTAYMELSSLRSEDTAVYYCARDYRKGLYAMDYWG |
| | | QGTTVTVSSASTKGPSVFPLAPCSRSTSESTAALGCLV |
| | | KDYFPEPVTVSWNSGALTSGVHTFPAVLQSSGLYSLSS |
| | | WTVPSSSLGTKTYTCNVDHKPSNTKVDKRVESKYGPP |
| | | CPPCPAPEFLGGPSVFLFPPKPKDTLMISRTPEVTCVV |
| | | VDVSQEDPEVQFNWYVDGVEVHNAKTKPREEQFNST |
| | | YRVVSVLTVLHQDWLNGKEYKCKVSNKGLPSSIEKTIS |
| | | KAKGQPREPQVYTLPPSQEEMTKNQVSLTCLVKGFYP |
| | | SDIAVEWESNGQPENNYKTTPPVLDSDGSFFLYSRLTV |
| | | DKSRWQEGNVFSCSVMHEALHNHYTQKSLSLSLG |
| SEQ ID NO: 623 | DNA | GAAGTGCAGCTGGTGCAGTCAGGCGCCGAAGTGAA |
| | heavy | GAAACCCGGCGCTACCGTGAAGATTAGCTGTAAAGT |
| | chain | CTCAGGCTACACCTTCACTAGCTACTGGATGTACTG |
| | | GGTCCGACAGGCTACCGGTCAAGGCCTGGAGTGGA |
| | | TGGGTAGAATCGACCCTAATAGCGGCTCTACTAAGT |
| | | ATAACGAGAAGTTTAAGAATAGAGTGACTATCACCGC |
| | | CGATAAGTCTACTAGCACCGCCTATATGGAACTGTCT |
| | | AGCCTGAGATCAGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| | | GCTAGCACTAAGGGCCCGTCCGTGTTCCCCCTGGCA |
| | | CCTTGTAGCCGGAGCACTAGCGAATCCACCGCTGCC |
| | | CTCGGCTGCCTGGTCAAGGATTACTTCCCGGAGCCC |
| | | GTGACCGTGTCCTGGAACAGCGGAGCCCTGACCTC |
| | | CGGAGTGCACACCTTCCCCGCTGTGCTGCAGAGCTC |
| | | CGGGCTGTACTCGCTGTCGTCGGTGGTCACGGTGC |
| | | CTTCATCTAGCCTGGGTACCAAGACCTACACTTGCAA |
| | | CGTGGACCACAAGCCTTCCAACACTAAGGTGGACAA |
| | | GCGCGTCGAATCGAAGTACGGCCCACCGTGCCCGC |
| | | CTTGTCCCGCGCCGGAGTTCCTCGGCGGTCCCTCG |
| | | GTCTTTCTGTTCCCACCGAAGCCCAAGGACACTTTGA |
| | | TGATTTCCCGCACCCCTGAAGTGACATGCGTGGTCG |
| | | TGGACGTGTCACAGGAAGATCCGGAGGTGCAGTTCA |
| | | ATTGGTACGTGGATGGCGTCGAGGTGCACAACGCCA |
| | | AAACCAAGCCGAGGGAGGAGCAGTTCAACTCCACTT |
| | | ACCGCGTCGTGTCCGTGCTGACGGTGCTGCATCAG |
| | | GACTGGCTGAACGGGAAGGAGTACAAGTGCAAAGTG |
| | | TCCAACAAGGGACTTCCTAGCTCAATCGAAAAGACC |
| | | ATCTCGAAAGCCAAGGGACAGCCCCGGGAACCCCA |
| | | AGTGTATACCCTGCCACCGAGCCAGGAAGAAATGAC |
| | | TAAGAACCAAGTCTCATTGACTTGCCTTGTGAAGGGC |
| | | TTCTACCCATCGGATATCGCCGTGGAATGGGAGTCC |
| | | AACGGCCAGCCGGAAAACAACTACAAGACCACCCCT |
| | | CCGGTGCTGGACTCAGACGGATCCTTCTTCCTCTAC |
| | | TCGCGGCTGACCGTGGATAAGAGCAGATGGCAGGA |
| | | GGGAAATGTGTTCAGCTGTTCTGTGATGCATGAAGC |
| | | CCTGCACAACCACTACACTCAGAAGTCCCTGTCCCT |
| | | CTCCCTGGGA |
| BAP058-Clone N LC | | |
| SEQ ID NO: 609 | LCDR1 | KASQDVGTAVA |
| (Kabat) | | |
| SEQ ID NO: 610 | LCDR2 | WASTRHT |
| (Kabat) | | |
| SEQ ID NO: 611 | LCDR3 | QQYNSYPLT |
| (Kabat) | | |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 624 | VL | DVVMTQSPLSLPVTLGQPASISCKASQDVGTAVAWYQ |
| | | QKPGQAPRLLIYWASTRHTGVPSRFSGSGSGTEFTLTI |
| | | SSLQPDDFATYYCQQYNSYPLTFGQGTKVEIK |
| SEQ ID NO: 625 | DNA VL | GACGTCGTGATGACTCAGTCACCCCTGAGCCTGCCC |
| | | GTGACCCTGGGGCAGCCCGCCTCTATTAGCTGTAAA |
| | | GCCTCTCAGGACGTGGGCACCGCCGTGGCCTGGTA |
| | | TCAGCAGAAGCCAGGGCAAGCCCCTAGACTGCTGAT |
| | | CTACTGGGCCTCTACTAGACACACCGGCGTGCCCTC |
| | | TAGGTTTAGCGGTAGCGGTAGTGGCACCGAGTTCAC |
| | | CCTGACTATCTCTTCACTGCAGCCCGACGACTTCGC |
| | | TACCTACTACTGTCAGCAGTATAATAGCTACCCCCTG |
| | | ACCTTCGGTCAAGGCACTAAGGTCGAGATTAAG |
| SEQ ID NO: 626 | Light | DVVMTQSPLSLPVTLGQPASISCKASQDVGTAVAWYQ |
| | chain | QKPGQAPRLLIYWASTRHTGVPSRFSGSGSGTEFTLTI |
| | | SSLQPDDFATYYCQQYNSYPLTFGQGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID NO: 627 | DNA light | GACGTCGTGATGACTCAGTCACCCCTGAGCCTGCCC |
| | chain | GTGACCCTGGGGCAGCCCGCCTCTATTAGCTGTAAA |
| | | GCCTCTCAGGACGTGGGCACCGCCGTGGCCTGGTA |
| | | TCAGCAGAAGCCAGGGCAAGCCCCTAGACTGCTGAT |
| | | CTACTGGGCCTCTACTAGACACACCGGCGTGCCCTC |
| | | TAGGTTTAGCGGTAGCGGTAGTGGCACCGAGTTCAC |
| | | CCTGACTATCTCTTCACTGCAGCCCGACGACTTCGC |
| | | TACCTACTACTGTCAGCAGTATAATAGCTACCCCCTG |
| | | ACCTTCGGTCAAGGCACTAAGGTCGAGATTAAGCGT |
| | | ACGGTGGCCGCTCCCAGCGTGTTCATCTTCCCCCCC |
| | | AGCGACGAGCAGCTGAAGAGCGGCACCGCCAGCGT |
| | | GGTGTGCCTGCTGAACAACTTCTACCCCCGGGAGGC |
| | | CAAGGTGCAGTGGAAGGTGGACAACGCCCTGCAGA |
| | | GCGGCAACAGCCAGGAGAGCGTCACCGAGCAGGAC |
| | | AGCAAGGACTCCACCTACAGCCTGAGCAGCACCCTG |
| | | ACCCTGAGCAAGGCCGACTACGAGAAGCATAAGGTG |
| | | TACGCCTGCGAGGTGACCCACCAGGGCCTGTCCAG |
| | | CCCCGTGACCAAGAGCTTCAACAGGGGCGAGTGC |
| BAP058-Clone O HC | | |
| SEQ ID NO: 628 | HCDR1 | agctactggatgtac |
| (Kabat) | | |
| SEQ ID NO: 629 | HCDR2 | agaatcgaccctaatagcggctctactaagtataacgagaagtttaagaat |
| (Kabat) | | |
| SEQ ID NO: 630 | HCDR3 | gactatagaaagggcctgtacgctatggactac |
| (Kabat) | | |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |
| BAP058-Clone O LC | | |
| SEQ ID NO: 633 | LCDR1 | aaagcctctcaggacgtgggcaccgccgtggcc |
| (Kabat) | | |
| SEQ ID NO: 634 | LCDR2 | tgggcctctactagacacacc |
| (Kabat) | | |
| SEQ ID NO: 635 | LCDR3 | cagcagtataatagctaccccctgacc |
| (Kabat) | | |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |
| BAP058-Clone N HC | | |
| SEQ ID NO: 628 | HCDR1 | agctactggatgtac |
| (Kabat) | | |
| SEQ ID NO: 629 | HCDR2 | agaatcgaccctaatagcggctctactaagtataacgagaagtttaagaat |
| (Kabat) | | |
| SEQ ID NO: 630 | HCDR3 | gactatagaaagggcctgtacgctatggactac |
| (Kabat) | | |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |
| BAP058-Clone N LC | | |
| SEQ ID NO: 633 | LCDR1 | aaagcctctcaggacgtgggcaccgccgtggcc |
| (Kabat) | | |
| SEQ ID NO: 634 | LCDR2 | tgggcctctactagacacacc |
| (Kabat) | | |
| SEQ ID NO: 635 | LCDR3 | cagcagtataatagctaccccctgacc |
| (Kabat) | | |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |

### Other Exemplary PD-L1 Inhibitors

In some embodiments, the PD-L1 inhibitor is anti-PD-L11 antibody. In some embodiments, the anti-PD-L11 inhibitor is selected from YW243.55.S70, MPDL3280A, MEDI-4736, or MDX-1105MSB-0010718C (also referred to as A09-246-2) disclosed in, e.g., WO 2013/0179174, and having a sequence disclosed herein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in PCT Publication No. WO 2007/005874.

In one embodiment, the PD-L1 inhibitor is YW243.55.S70. The YW243.55.S70 antibody is an anti-PD-L1 described in PCT Publication No. WO 2010/077634.

In one embodiment, the PD-L1 inhibitor is MDPL3280A (Genentech/Roche) also known as Atezolizumabm, RG7446, RO5541267, YW243.55.S70, or TECENTRIQ™. MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906 incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab, e.g., as disclosed in Table 7.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in PCT Publication Nos. WO2010/027827 and WO2011/066342).

In one embodiment the PD-L1 inhibitor is an anti-PD-L1 antibody molecule. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 7.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 7.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 7.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 7
Amino acid sequences of other exemplary anti-PD-L1 antibody molecules

| | | |
|---|---|---|
| Atezolizumab | | |
| SEQ ID NO: 639 | Heavy | EVQLVESGGGLVQPGGSLRLSCAASFTFSDSWIHWVRQAPGKG |
| | chain | LEWVAWISPYGGSTYYADSVKGRFTISADTSKNTAYLQMNSLRAED |
| | | TAVYYCARRHWPGGFDYWGQGTLVTVSSASTKGPSVFPLAPSSK |
| | | STSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHED |
| | | PEVKFNWYVDGVEVHNAKTKPREEQYASTYRVVSVLTVLHQDWLN |
| | | GKEYKCKVSNKALPAPIEKTISKAKGQPREPQVYTLPPSREEMTKN |
| | | QVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| SEQ ID NO: 640 | Light | DIQMTQSPSSLSASVGDRVTITCRASQDVSTAVAWYQQKPGKAPK |
| | chain | LLIYSASFLYSGVPSRFSGSGSGTDFTLTISSLQPEDFATYYCQQYL |
| | | YHPATFGQGTKVEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |
| Avelumab | | |
| SEQ ID NO: 641 | Heavy | EVQLLESGGGLVQPGGSLRLSCAASGFTFSSYIMMWVRQAPGKGL |
| | chain | EWVSSIYPSGGITFYADTVKGRFTISRDNSKNTLYLQMNSLRAEDTA |
| | | VYYCARIKLGTVTTVDYWGQGTLVTVSSASTKGPSVFPLAPSSKST |
| | | SGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVMHEALHNHYTQKSLSLSPGK |
| SEQ ID NO: 642 | Light | QSALTQPASVSGSPGQSITISCTGTSSDVGGYNYVSWYQQHPGKA |
| | chain | PKLMIYDVSNRPSGVSNRFSGSKSGNTASLTISGLQAEDEADYYCS |
| | | SYTSSSTRVFGTGTKVTVLGQPKANPTVTLFPPSSEELQANKATLV |
| | | CLISDFYPGAVTVAWKADGSPVKAGVETTKPSKQSNNKYAASSYLS |
| | | LTPEQWKSHRSYSCQVTHEGSTVEKTVAPTECS |
| Durvalumab | | |
| SEQ ID NO: 643 | Heavy | EVQLVESGGGLVQPGGSLRLSCAASGFTFSRYWMSWVRQAPGKG |
| | chain | LEWVANIKQDGSEKYYVDSVKGRFTISRDNAKNSLYLQMNSLRAED |
| | | TAVYYCAREGGWFGELAFDYWGQGTLVTVSSASTKGPSVFPLAPS |
| | | SKSTSGGTAALGCLVKDYFPEPVTVSWNSGALTSGVHTFPAVLQS |
| | | SGLYSLSSVTVPSSSLGTQTYICNVNHKPSNTKVDKRVEPKSCDK |
| | | THTCPPCPAPEFEGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPASIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| SEQ ID NO: 644 | Light | EIVLTQSPGTLSLSPGERATLSCRASQRVSSSYLAWYQQKPGQAP |
| | chain | RLLIYDASSRATGIPDRFSGSGSGTDFTLTISRLEPEDFAVYYCQQY |
| | | GSLPWTFGQGTKVEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| BMS-936559 | | |
| SEQ ID NO: 645 | VH | QVQLVQSGAEVKKPGSSVKVSCKTSGDTFSTYAISWVRQAPGQGL |
| | | EWMGGIIPIFGKAHYAQKFQGRVTITADESTSTAYMELSSLRSEDTA |
| | | VYFCARKFHFVSGSPFGMDVWGQGTTVTVSS |
| SEQ ID NO: 646 | VL | EIVLTQSPATLSLSPGERATLSCRASQSVSSYLAWYQQKPGQAPRL |
| | | LIYDASNRATGIPARFSGSGSGTDFTLTISSLEPEDFAVYYCQQRSN |
| | | WPTFGQGTKVEIK |

### LAG-3 Inhibitors

In certain embodiments, the inhibitor of an immune checkpoint molecule is an inhibitor of LAG-3. In some embodiments, the antibody conjugate of the present invention is administered in combination with a LAG-3 inhibitor. In some embodiments, the LAG-3 inhibitor is selected from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 8 (e.g., from the heavy and light chain variable region sequences of BAP050-Clone I or BAP050-Clone J disclosed in Table 8), or encoded by a nucleotide sequence shown in Table 8. In some embodiments, the CDRs are according to the Kabat definition (e.g., as set out in Table 8). In some embodiments, the CDRs are according to the Chothia definition (e.g., as set out in Table 8). In some embodiments, the CDRs are according to the combined CDR definitions of both Kabat and Chothia (e.g., as set out in Table 8). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 8, or encoded by a nucleotide sequence shown in Table 8.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 8.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 8. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 8.

In one embodiment, the anti-LAG-3 antibody molecule comprises a VH comprising the amino acid sequence of SEQ ID NO: 706, or an amino acid sequence at least 85%, 90%, 95%, or 99% identical or higher to SEQ ID NO: 706. In one embodiment, the anti-LAG-3 antibody molecule comprises a VL comprising the amino acid sequence of SEQ ID NO: 718, or an amino acid sequence at least 85%, 90%, 95%, or 99% identical or higher to SEQ ID NO: 718. In one embodiment, the anti-LAG-3 antibody molecule comprises a VH comprising the amino acid sequence of SEQ ID NO: 724, or an amino acid sequence at least 85%, 90%, 95%, or 99% identical or higher to SEQ ID NO: 724. In one embodiment, the anti-LAG-3 antibody molecule comprises a VL comprising the amino acid sequence of SEQ ID NO: 730, or an amino acid sequence at least 85%, 90%, 95%, or 99% identical or higher to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence at least 85%, 90%, 95%, or 99% identical or higher to SEQ ID NO: 707 or 708. In one embodiment, the antibody molecule comprises a VL encoded by the nucleotide sequence of SEQ ID NO: 719 or 720, or a nucleotide sequence at least 85%, 90%, 95%, or 99% identical or higher to SEQ ID NO: 719 or 720. In one embodiment, the antibody molecule comprises a VH encoded by the nucleotide sequence of SEQ ID NO: 725 or 726, or a nucleotide sequence at least 85%, 90%, 95%, or 99% identical or higher to SEQ ID NO: 725 or 726. In one embodiment, the antibody molecule comprises a VL encoded by the nucleotide sequence of SEQ ID NO: 731 or 732, or a nucleotide sequence at least 85%, 90%, 95%, or 99% identical or higher to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence at least 85%, 90%, 95%, or 99% identical or higher to SEQ ID NO: 709. In one embodiment, the anti-LAG-3 antibody molecule comprises a light chain comprising the amino acid sequence of SEQ ID NO: 721, or an amino acid sequence at least 85%, 90%, 95%, or 99% identical or higher to SEQ ID NO: 721. In one embodiment, the anti-LAG-3 antibody molecule comprises a heavy chain comprising the amino acid sequence of SEQ ID NO: 727, or an amino acid sequence at least 85%, 90%, 95%, or 99% identical or higher to SEQ ID NO: 727. In one embodiment, the anti-LAG-3 antibody molecule comprises a light chain comprising the amino acid sequence of SEQ ID NO: 733, or an amino acid sequence at least 85%, 90%, 95%, or 99% identical or higher to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence at least 85%, 90%, 95%, or 99% identical or higher to SEQ ID NO: 716 or 717. In one embodiment, the antibody molecule comprises a light chain encoded by the nucleotide sequence of SEQ ID NO: 722 or 723, or a nucleotide sequence at least 85%, 90%, 95%, or 99% identical or higher to SEQ ID NO: 722 or 723. In one embodiment, the antibody molecule comprises a heavy chain encoded by the nucleotide sequence of SEQ ID NO: 728 or 729, or a nucleotide sequence at least 85%, 90%, 95%, or 99% identical or higher to SEQ ID NO: 728 or 729. In one embodiment, the antibody molecule comprises a light chain encoded by the nucleotide sequence of SEQ ID NO: 734 or 735, or a nucleotide sequence at least 85%, 90%, 95%, or 99% identical or higher to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 8
Amino acid and nucleotide sequences
of exemplary anti-LAG-3 antibody molecules

| | | |
|---|---|---|
| BAP050-Clone I HC | | |
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 706 | VH | QVQLVQSGAEVKKPGASVKVSCKASGFTLTNYGMNWVRQ |
| | | ARGQRLEWIGWINTDTGEPTYADDFKGRFVFSLDTSVSTA |
| | | YLQISSLKAEDTAVYYCARNPPYYYGTNNAEAMDYWGQG |
| | | TTVTVSS |
| SEQ ID NO: 707 | DNA VH | CAAGTGCAGCTGGTGCAGTCGGGAGCCGAAGTGAAGAA |
| | | GCCTGGAGCCTCGGTGAAGGTGTCGTGCAAGGCATCCG |
| | | GATTCACCCTCACCAATTACGGGATGAACTGGGTCAGAC |
| | | AGGCCCGGGGTCAACGGCTGGAGTGGATCGGATGGATT |
| | | AACACCGACACCGGGGAGCCTACCTACGCGGACGATTT |
| | | CAAGGGACGGTTCGTGTTCTCCCTCGACACCTCCGTGT |
| | | CCACCGCCTACCTCCAAATCTCCTCACTGAAAGCGGAG |
| | | GACACCGCCGTGTACTATTGCGCGAGGAACCCGCCCTA |
| | | CTACTACGGAACCAACAACGCCGAAGCCATGGACTACT |
| | | GGGGCCAGGGCACCACTGTGACTGTGTCCAGC |
| SEQ ID NO: 708 | DNA VH | CAGGTGCAGCTGGTGCAGTCTGGCGCCGAAGTGAAGAA |
| | | ACCTGGCGCCTCCGTGAAGGTGTCCTGCAAGGCCTCTG |
| | | GCTTCACCCTGACCAACTACGGCATGAACTGGGTGCGA |
| | | CAGGCCAGGGGCCAGCGGCTGGAATGGATCGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCT |
| SEQ ID NO: 709 | Heavy | QVQLVQSGAEVKKPGASVKVSCKASGFTLTNYGMNWVRQ |
| | chain | ARGQRLEWIGWINTDTGEPTYADDFKGRFVFSLDTSVSTA |
| | | YLQISSLKAEDTAVYYCARNPPYYYGTNNAEAMDYWGQG |
| | | TTVTVSSASTKGPSVFPLAPCSRSTSESTAALGCLVKDYFP |
| | | EPVTVSWNSGALTSGVHTFPAVLQSSGLYSLSSWTVPSS |
| | | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDWL |
| | | NGKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHN |
| | | HYTQKSLSLSLG |
| SEQ ID NO: 716 | DNA | CAAGTGCAGCTGGTGCAGTCGGGAGCCGAAGTGAAGAA |
| | heavy | GCCTGGAGCCTCGGTGAAGGTGTCGTGCAAGGCATCCG |
| | chain | GATTCACCCTCACCAATTACGGGATGAACTGGGTCAGAC |
| | | AGGCCCGGGGTCAACGGCTGGAGTGGATCGGATGGATT |
| | | AACACCGACACCGGGGAGCCTACCTACGCGGACGATTT |
| | | CAAGGGACGGTTCGTGTTCTCCCTCGACACCTCCGTGT |
| | | CCACCGCCTACCTCCAAATCTCCTCACTGAAAGCGGAG |
| | | GACACCGCCGTGTACTATTGCGCGAGGAACCCGCCCTA |
| | | CTACTACGGAACCAACAACGCCGAAGCCATGGACTACT |
| | | GGGGCCAGGGCACCACTGTGACTGTGTCCAGCGCGTC |
| | | CACTAAGGGCCCGTCCGTGTTCCCCCTGGCACCTTGTA |
| | | GCCGGAGCACTAGCGAATCCACCGCTGCCCTCGGCTGC |
| | | CTGGTCAAGGATTACTTCCCGGAGCCCGTGACCGTGTC |
| | | CTGGAACAGCGGAGCCCTGACCTCCGGAGTGCACACCT |
| | | TCCCCGCTGTGCTGCAGAGCTCCGGGCTGTACTCGCTG |
| | | TCGTCGGTGGTCACGGTGCCTTCATCTAGCCTGGGTAC |
| | | CAAGACCTACACTTGCAACGTGGACCACAAGCCTTCCAA |
| | | CACTAAGGTGGACAAGCGCGTCGAATCGAAGTACGGCC |
| | | CACCGTGCCCGCCTTGTCCCGCGCCGGAGTTCCTCGGC |
| | | GGTCCCTCGGTCTTTCTGTTCCCACCGAAGCCCAAGGA |
| | | CACTTTGATGATTTCCCGCACCCCTGAAGTGACATGCGT |
| | | GGTCGTGGACGTGTCACAGGAAGATCCGGAGGTGCAGT |
| | | TCAATTGGTACGTGGATGGCGTCGAGGTGCACAACGCC |
| | | AAAACCAAGCCGAGGGAGGAGCAGTTCAACTCCACTTA |
| | | CCGCGTCGTGTCCGTGCTGACGGTGCTGCATCAGGACT |
| | | GGCTGAACGGGAAGGAGTACAAGTGCAAAGTGTCCAAC |
| | | AAGGGACTTCCTAGCTCAATCGAAAAGACCATCTCGAAA |
| | | GCCAAGGGACAGCCCCGGGAACCCCAAGTGTATACCCT |
| | | GCCACCGAGCCAGGAAGAAATGACTAAGAACCAAGTCT |
| | | CATTGACTTGCCTTGTGAAGGGCTTCTACCCATCGGATA |
| | | TCGCCGTGGAATGGGAGTCCAACGGCCAGCCGGAAAAC |
| | | AACTACAAGACCACCCCTCCGGTGCTGGACTCAGACGG |
| | | ATCCTTCTTCCTCTACTCGCGGCTGACCGTGGATAAGAG |
| | | CAGATGGCAGGAGGGAAATGTGTTCAGCTGTTCTGTGAT |
| | | GCATGAAGCCCTGCACAACCACTACACTCAGAAGTCCCT |
| | | GTCCCTCTCCCTGGGA |
| SEQ ID NO: 717 | DNA | CAGGTGCAGCTGGTGCAGTCTGGCGCCGAAGTGAAGAA |
| | heavy | ACCTGGCGCCTCCGTGAAGGTGTCCTGCAAGGCCTCTG |
| | chain | GCTTCACCCTGACCAACTACGGCATGAACTGGGTGCGA |
| | | CAGGCCAGGGGCCAGCGGCTGGAATGGATCGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCTGCTTC |
| | | TACCAAGGGGCCCAGCGTGTTCCCCCTGGCCCCCTGCT |
| | | CCAGAAGCACCAGCGAGAGCACAGCCGCCCTGGGCTG |
| | | CCTGGTGAAGGACTACTTCCCCGAGCCCGTGACCGTGT |
| | | CCTGGAACAGCGGAGCCCTGACCAGCGGCGTGCACAC |
| | | CTTCCCCGCCGTGCTGCAGAGCAGCGGCCTGTACAGCC |
| | | TGAGCAGCGTGGTGACCGTGCCCAGCAGCAGCCTGGG |
| | | CACCAAGACCTACACCTGTAACGTGGACCACAAGCCCA |
| | | GCAACACCAAGGTGGACAAGAGGGTGGAGAGCAAGTAC |
| | | GGCCCACCCTGCCCCCCCTGCCCAGCCCCCGAGTTCCT |
| | | GGGCGGACCCAGCGTGTTCCTGTTCCCCCCCAAGCCCA |
| | | AGGACACCCTGATGATCAGCAGAACCCCCGAGGTGACC |
| | | TGTGTGGTGGTGGACGTGTCCCAGGAGGACCCCGAGGT |
| | | CCAGTTCAACTGGTACGTGGACGGCGTGGAGGTGCACA |
| | | ACGCCAAGACCAAGCCCAGAGAGGAGCAGTTTAACAGC |
| | | ACCTACCGGGTGGTGTCCGTGCTGACCGTGCTGCACCA |
| | | GGACTGGCTGAACGGCAAAGAGTACAAGTGTAAGGTCT |
| | | CCAACAAGGGCCTGCCAAGCAGCATCGAAAAGACCATC |
| | | AGCAAGGCCAAGGGCCAGCCTAGAGAGCCCCAGGTCTA |
| | | CACCCTGCCACCCAGCCAAGAGGAGATGACCAAGAACC |
| | | AGGTGTCCCTGACCTGTCTGGTGAAGGGCTTCTACCCA |
| | | AGCGACATCGCCGTGGAGTGGGAGAGCAACGGCCAGC |
| | | CCGAGAACAACTACAAGACCACCCCCCCAGTGCTGGAC |
| | | AGCGACGGCAGCTTCTTCCTGTACAGCAGGCTGACCGT |
| | | GGACAAGTCCAGATGGCAGGAGGGCAACGTCTTTAGCT |
| | | GCTCCGTGATGCACGAGGCCCTGCACAACCACTACACC |
| | | CAGAAGAGCCTGAGCCTGTCCCTGGGC |
| BAP050-Clone I LC | | |
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 718 | VL | DIQMTQSPSSLSASVGDRVTITCSSSQDISNYLNWYLQKPG |
| | | QSPQLLIYYTSTLHLGVPSRFSGSGSGTEFTLTISSLQPDDF |
| | | ATYYCQQYYNLPWTFGQGTKVEIK |
| SEQ ID NO: 719 | DNA VL | GATATTCAGATGACTCAGTCACCTAGTAGCCTGAGCGCT |
| | | AGTGTGGGCGATAGAGTGACTATCACCTGTAGCTCTAGT |
| | | CAGGATATCTCTAACTACCTGAACTGGTATCTGCAGAAG |
| | | CCCGGTCAATCACCTCAGCTGCTGATCTACTACACTAGC |
| | | ACCCTGCACCTGGGCGTGCCCTCTAGGTTTAGCGGTAG |
| | | CGGTAGTGGCACCGAGTTCACCCTGACTATCTCTAGCCT |
| | | GCAGCCCGACGACTTCGCTACCTACTACTGTCAGCAGTA |
| | | CTATAACCTGCCCTGGACCTTCGGTCAAGGCACTAAGGT |
| | | CGAGATTAAG |
| SEQ ID NO: 720 | DNA VL | GACATCCAGATGACCCAGTCCCCCTCCAGCCTGTCTGC |
| | | TTCCGTGGGCGACAGAGTGACCATCACCTGTTCCTCCA |
| | | GCCAGGACATCTCCAACTACCTGAACTGGTATCTGCAGA |
| | | AGCCCGGCCAGTCCCCTCAGCTGCTGATCTACTACACC |
| | | TCCACCCTGCACCTGGGCGTGCCCTCCAGATTTTCCGG |
| | | CTCTGGCTCTGGCACCGAGTTTACCCTGACCATCAGCTC |
| | | CCTGCAGCCCGACGACTTCGCCACCTACTACTGCCAGC |
| | | AGTACTACAACCTGCCCTGGACCTTCGGCCAGGGCACC |
| | | AAGGTGGAAATCAAG |
| SEQ ID NO: 721 | Light | DIQMTQSPSSLSASVGDRVTITCSSSQDISNYLNWYLQKPG |
| | chain | QSPQLLIYYTSTLHLGVPSRFSGSGSGTEFTLTISSLQPDDF |
| | | ATYYCQQYYNLPWTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| SEQ ID NO: 722 | DNA | GATATTCAGATGACTCAGTCACCTAGTAGCCTGAGCGCT |
| | light | AGTGTGGGCGATAGAGTGACTATCACCTGTAGCTCTAGT |
| | chain | CAGGATATCTCTAACTACCTGAACTGGTATCTGCAGAAG |
| | | CCCGGTCAATCACCTCAGCTGCTGATCTACTACACTAGC |
| | | ACCCTGCACCTGGGCGTGCCCTCTAGGTTTAGCGGTAG |
| | | CGGTAGTGGCACCGAGTTCACCCTGACTATCTCTAGCCT |
| | | GCAGCCCGACGACTTCGCTACCTACTACTGTCAGCAGTA |
| | | CTATAACCTGCCCTGGACCTTCGGTCAAGGCACTAAGGT |
| | | CGAGATTAAGCGTACGGTGGCCGCTCCCAGCGTGTTCA |
| | | TCTTCCCCCCCAGCGACGAGCAGCTGAAGAGCGGCACC |
| | | GCCAGCGTGGTGTGCCTGCTGAACAACTTCTACCCCCG |
| | | GGAGGCCAAGGTGCAGTGGAAGGTGGACAACGCCCTG |
| | | CAGAGCGGCAACAGCCAGGAGAGCGTCACCGAGCAGG |
| | | ACAGCAAGGACTCCACCTACAGCCTGAGCAGCACCCTG |
| | | ACCCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| SEQ ID NO: 723 | DNA | GACATCCAGATGACCCAGTCCCCCTCCAGCCTGTCTGC |
| | light | TTCCGTGGGCGACAGAGTGACCATCACCTGTTCCTCCA |
| | chain | GCCAGGACATCTCCAACTACCTGAACTGGTATCTGCAGA |
| | | AGCCCGGCCAGTCCCCTCAGCTGCTGATCTACTACACC |
| | | TCCACCCTGCACCTGGGCGTGCCCTCCAGATTTTCCGG |
| | | CTCTGGCTCTGGCACCGAGTTTACCCTGACCATCAGCTC |
| | | CCTGCAGCCCGACGACTTCGCCACCTACTACTGCCAGC |
| | | AGTACTACAACCTGCCCTGGACCTTCGGCCAGGGCACC |
| | | AAGGTGGAAATCAAGCGTACGGTGGCCGCTCCCAGCGT |
| | | GTTCATCTTCCCCCCAAGCGACGAGCAGCTGAAGAGCG |
| | | GCACCGCCAGCGTGGTGTGTCTGCTGAACAACTTCTAC |
| | | CCCAGGGAGGCCAAGGTGCAGTGGAAGGTGGACAACG |
| | | CCCTGCAGAGCGGCAACAGCCAGGAGAGCGTCACCGA |
| | | GCAGGACAGCAAGGACTCCACCTACAGCCTGAGCAGCA |
| | | CCCTGACCCTGAGCAAGGCCGACTACGAGAAGCACAAG |
| | | GTGTACGCCTGTGAGGTGACCCACCAGGGCCTGTCCAG |
| | | CCCCGTGACCAAGAGCTTCAACAGGGGCGAGTGC |
| BAP050-Clone J HC | | |
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNYGMNWVRQ |
| | | APGQGLEWMGWINTDTGEPTYADDFKGRFVFSLDTSVST |
| | | AYLQISSLKAEDTAVYYCARNPPYYYGTNNAEAMDYWGQ |
| | | GTTVTVSS |
| SEQ ID NO: 725 | DNA VH | CAGGTGCAGCTGGTGCAGTCAGGCGCCGAAGTGAAGAA |
| | | ACCCGGCGCTAGTGTGAAAGTCAGCTGTAAAGCTAGTG |
| | | GCTTCACCCTGACTAACTACGGGATGAACTGGGTCCGC |
| | | CAGGCCCCAGGTCAAGGCCTCGAGTGGATGGGCTGGAT |
| | | TAACACCGACACCGGCGAGCCTACCTACGCCGACGACT |
| | | TTAAGGGCAGATTCGTGTTTAGCCTGGACACTAGTGTGT |
| | | CTACCGCCTACCTGCAGATCTCTAGCCTGAAGGCCGAG |
| | | GACACCGCCGTCTACTACTGCGCTAGAAACCCCCCCTA |
| | | CTACTACGGCACTAACAACGCCGAGGCTATGGACTACT |
| | | GGGGTCAAGGCACTACCGTGACCGTGTCTAGC |
| SEQ ID NO: 726 | DNA VH | CAGGTGCAGCTGGTGCAGTCTGGCGCCGAAGTGAAGAA |
| | | ACCTGGCGCCTCCGTGAAGGTGTCCTGCAAGGCCTCTG |
| | | GCTTCACCCTGACCAACTACGGCATGAACTGGGTGCGA |
| | | CAGGCCCCTGGACAGGGCCTGGAATGGATGGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCT |
| SEQ ID NO: 727 | Heavy | QVQLVQSGAEVKKPGASVKVSCKASGFTLTNYGMNWVRQ |
| | chain | APGQGLEWMGWINTDTGEPTYADDFKGRFVFSLDTSVST |
| | | AYLQISSLKAEDTAVYYCARNPPYYYGTNNAEAMDYWGQ |
| | | GTTVTVSSASTKGPSVFPLAPCSRSTSESTAALGCLVKDYF |
| | | PEPVTVSWNSGALTSGVHTFPAVLQSSGLYSLSSWTVPS |
| | | SSLGTKTYTCNVDHKPSNTKVDKRVESKYGPPCPPCPAPE |
| | | FLGGPSVFLFPPKPKDTLMISRTPEVTCVVVDVSQEDPEVQ |
| | | FNWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKTT |
| | | PPVLDSDGSFFLYSRLTVDKSRWQEGNVFSCSVMHEALH |
| | | NHYTQKSLSLSLG |
| SEQ ID NO: 728 | DNA | CAGGTGCAGCTGGTGCAGTCAGGCGCCGAAGTGAAGAA |
| | heavy | ACCCGGCGCTAGTGTGAAAGTCAGCTGTAAAGCTAGTG |
| | chain | GCTTCACCCTGACTAACTACGGGATGAACTGGGTCCGC |
| | | CAGGCCCCAGGTCAAGGCCTCGAGTGGATGGGCTGGAT |
| | | TAACACCGACACCGGCGAGCCTACCTACGCCGACGACT |
| | | TTAAGGGCAGATTCGTGTTTAGCCTGGACACTAGTGTGT |
| | | CTACCGCCTACCTGCAGATCTCTAGCCTGAAGGCCGAG |
| | | GACACCGCCGTCTACTACTGCGCTAGAAACCCCCCCTA |
| | | CTACTACGGCACTAACAACGCCGAGGCTATGGACTACT |
| | | GGGGTCAAGGCACTACCGTGACCGTGTCTAGCGCTAGC |
| | | ACTAAGGGCCCGTCCGTGTTCCCCCTGGCACCTTGTAG |
| | | CCGGAGCACTAGCGAATCCACCGCTGCCCTCGGCTGCC |
| | | TGGTCAAGGATTACTTCCCGGAGCCCGTGACCGTGTCC |
| | | TGGAACAGCGGAGCCCTGACCTCCGGAGTGCACACCTT |
| | | CCCCGCTGTGCTGCAGAGCTCCGGGCTGTACTCGCTGT |
| | | CGTCGGTGGTCACGGTGCCTTCATCTAGCCTGGGTACC |
| | | AAGACCTACACTTGCAACGTGGACCACAAGCCTTCCAAC |
| | | ACTAAGGTGGACAAGCGCGTCGAATCGAAGTACGGCCC |
| | | ACCGTGCCCGCCTTGTCCCGCGCCGGAGTTCCTCGGCG |
| | | GTCCCTCGGTCTTTCTGTTCCCACCGAAGCCCAAGGACA |
| | | CTTTGATGATTTCCCGCACCCCTGAAGTGACATGCGTGG |
| | | TCGTGGACGTGTCACAGGAAGATCCGGAGGTGCAGTTC |
| | | AATTGGTACGTGGATGGCGTCGAGGTGCACAACGCCAA |
| | | AACCAAGCCGAGGGAGGAGCAGTTCAACTCCACTTACC |
| | | GCGTCGTGTCCGTGCTGACGGTGCTGCATCAGGACTGG |
| | | CTGAACGGGAAGGAGTACAAGTGCAAAGTGTCCAACAA |
| | | GGGACTTCCTAGCTCAATCGAAAAGACCATCTCGAAAGC |
| | | CAAGGGACAGCCCCGGGAACCCCAAGTGTATACCCTGC |
| | | CACCGAGCCAGGAAGAAATGACTAAGAACCAAGTCTCAT |
| | | TGACTTGCCTTGTGAAGGGCTTCTACCCATCGGATATCG |
| | | CCGTGGAATGGGAGTCCAACGGCCAGCCGGAAAACAAC |
| | | TACAAGACCACCCCTCCGGTGCTGGACTCAGACGGATC |
| | | CTTCTTCCTCTACTCGCGGCTGACCGTGGATAAGAGCAG |
| | | ATGGCAGGAGGGAAATGTGTTCAGCTGTTCTGTGATGCA |
| | | TGAAGCCCTGCACAACCACTACACTCAGAAGTCCCTGTC |
| | | CCTCTCCCTGGGA |
| SEQ ID NO: 729 | DNA | CAGGTGCAGCTGGTGCAGTCTGGCGCCGAAGTGAAGAA |
| | heavy | ACCTGGCGCCTCCGTGAAGGTGTCCTGCAAGGCCTCTG |
| | chain | GCTTCACCCTGACCAACTACGGCATGAACTGGGTGCGA |
| | | CAGGCCCCTGGACAGGGCCTGGAATGGATGGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCTGCTTC |
| | | TACCAAGGGGCCCAGCGTGTTCCCCCTGGCCCCCTGCT |
| | | CCAGAAGCACCAGCGAGAGCACAGCCGCCCTGGGCTG |
| | | CCTGGTGAAGGACTACTTCCCCGAGCCCGTGACCGTGT |
| | | CCTGGAACAGCGGAGCCCTGACCAGCGGCGTGCACAC |
| | | CTTCCCCGCCGTGCTGCAGAGCAGCGGCCTGTACAGCC |
| | | TGAGCAGCGTGGTGACCGTGCCCAGCAGCAGCCTGGG |
| | | CACCAAGACCTACACCTGTAACGTGGACCACAAGCCCA |
| | | GCAACACCAAGGTGGACAAGAGGGTGGAGAGCAAGTAC |
| | | GGCCCACCCTGCCCCCCCTGCCCAGCCCCCGAGTTCCT |
| | | GGGCGGACCCAGCGTGTTCCTGTTCCCCCCCAAGCCCA |
| | | AGGACACCCTGATGATCAGCAGAACCCCCGAGGTGACC |
| | | TGTGTGGTGGTGGACGTGTCCCAGGAGGACCCCGAGGT |
| | | CCAGTTCAACTGGTACGTGGACGGCGTGGAGGTGCACA |
| | | ACGCCAAGACCAAGCCCAGAGAGGAGCAGTTTAACAGC |
| | | ACCTACCGGGTGGTGTCCGTGCTGACCGTGCTGCACCA |
| | | GGACTGGCTGAACGGCAAAGAGTACAAGTGTAAGGTCT |
| | | CCAACAAGGGCCTGCCAAGCAGCATCGAAAAGACCATC |
| | | AGCAAGGCCAAGGGCCAGCCTAGAGAGCCCCAGGTCTA |
| | | CACCCTGCCACCCAGCCAAGAGGAGATGACCAAGAACC |
| | | AGGTGTCCCTGACCTGTCTGGTGAAGGGCTTCTACCCA |
| | | AGCGACATCGCCGTGGAGTGGGAGAGCAACGGCCAGC |
| | | CCGAGAACAACTACAAGACCACCCCCCCAGTGCTGGAC |
| | | AGCGACGGCAGCTTCTTCCTGTACAGCAGGCTGACCGT |
| | | GGACAAGTCCAGATGGCAGGAGGGCAACGTCTTTAGCT |
| | | GCTCCGTGATGCACGAGGCCCTGCACAACCACTACACC |
| | | CAGAAGAGCCTGAGCCTGTCCCTGGGC |
| BAP050-Clone J LC | | |
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQMTQSPSSLSASVGDRVTITCSSSQDISNYLNWYQQKP |
| | | GKAPKLLIYYTSTLHLGIPPRFSGSGYGTDFTLTINNIESEDA |
| | | AYYFCQQYYNLPWTFGQGTKVEIK |
| SEQ ID NO: 731 | DNA VL | GATATTCAGATGACTCAGTCACCTAGTAGCCTGAGCGCT |
| | | AGTGTGGGCGATAGAGTGACTATCACCTGTAGCTCTAGT |
| | | CAGGATATCTCTAACTACCTGAACTGGTATCAGCAGAAG |
| | | CCCGGTAAAGCCCCTAAGCTGCTGATCTACTACACTAGC |
| | | ACCCTGCACCTGGGAATCCCCCCTAGGTTTAGCGGTAG |
| | | CGGCTACGGCACCGACTTCACCCTGACTATTAACAATAT |
| | | CGAGTCAGAGGACGCCGCCTACTACTTCTGTCAGCAGT |
| | | ACTATAACCTGCCCTGGACCTTCGGTCAAGGCACTAAGG |
| | | TCGAGATTAAG |
| SEQ ID NO: 732 | DNA VL | GACATCCAGATGACCCAGTCCCCCTCCAGCCTGTCTGC |
| | | TTCCGTGGGCGACAGAGTGACCATCACCTGTTCCTCCA |
| | | GCCAGGACATCTCCAACTACCTGAACTGGTATCAGCAGA |
| | | AGCCCGGCAAGGCCCCCAAGCTGCTGATCTACTACACC |
| | | TCCACCCTGCACCTGGGCATCCCCCCTAGATTCTCCGG |
| | | CTCTGGCTACGGCACCGACTTCACCCTGACCATCAACAA |
| | | CATCGAGTCCGAGGACGCCGCCTACTACTTCTGCCAGC |
| | | AGTACTACAACCTGCCCTGGACCTTCGGCCAGGGCACC |
| | | AAGGTGGAAATCAAG |
| SEQ ID NO: 733 | Light | DIQMTQSPSSLSASVGDRVTITCSSSQDISNYLNWYQQKP |
| | chain | GKAPKLLIYYTSTLHLGIPPRFSGSGYGTDFTLTINNIESEDA |
| | | AYYFCQQYYNLPWTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| SEQ ID NO: 734 | DNA | GATATTCAGATGACTCAGTCACCTAGTAGCCTGAGCGCT |
| | light | AGTGTGGGCGATAGAGTGACTATCACCTGTAGCTCTAGT |
| | chain | CAGGATATCTCTAACTACCTGAACTGGTATCAGCAGAAG |
| | | CCCGGTAAAGCCCCTAAGCTGCTGATCTACTACACTAGC |
| | | ACCCTGCACCTGGGAATCCCCCCTAGGTTTAGCGGTAG |
| | | CGGCTACGGCACCGACTTCACCCTGACTATTAACAATAT |
| | | CGAGTCAGAGGACGCCGCCTACTACTTCTGTCAGCAGT |
| | | ACTATAACCTGCCCTGGACCTTCGGTCAAGGCACTAAGG |
| | | TCGAGATTAAGCGTACGGTGGCCGCTCCCAGCGTGTTC |
| | | ATCTTCCCCCCCAGCGACGAGCAGCTGAAGAGCGGCAC |
| | | CGCCAGCGTGGTGTGCCTGCTGAACAACTTCTACCCCC |
| | | GGGAGGCCAAGGTGCAGTGGAAGGTGGACAACGCCCT |
| | | GCAGAGCGGCAACAGCCAGGAGAGCGTCACCGAGCAG |
| | | GACAGCAAGGACTCCACCTACAGCCTGAGCAGCACCCT |
| | | GACCCTGAGCAAGGCCGACTACGAGAAGCATAAGGTGT |
| | | ACGCCTGCGAGGTGACCCACCAGGGCCTGTCCAGCCC |
| | | CGTGACCAAGAGCTTCAACAGGGGCGAGTGC |
| SEQ ID NO: 735 | DNA | GACATCCAGATGACCCAGTCCCCCTCCAGCCTGTCTGC |
| | light | TTCCGTGGGCGACAGAGTGACCATCACCTGTTCCTCCA |
| | chain | GCCAGGACATCTCCAACTACCTGAACTGGTATCAGCAGA |
| | | AGCCCGGCAAGGCCCCCAAGCTGCTGATCTACTACACC |
| | | TCCACCCTGCACCTGGGCATCCCCCCTAGATTCTCCGG |
| | | CTCTGGCTACGGCACCGACTTCACCCTGACCATCAACAA |
| | | CATCGAGTCCGAGGACGCCGCCTACTACTTCTGCCAGC |
| | | AGTACTACAACCTGCCCTGGACCTTCGGCCAGGGCACC |
| | | AAGGTGGAAATCAAGCGTACGGTGGCCGCTCCCAGCGT |
| | | GTTCATCTTCCCCCCAAGCGACGAGCAGCTGAAGAGCG |
| | | GCACCGCCAGCGTGGTGTGTCTGCTGAACAACTTCTAC |
| | | CCCAGGGAGGCCAAGGTGCAGTGGAAGGTGGACAACG |
| | | CCCTGCAGAGCGGCAACAGCCAGGAGAGCGTCACCGA |
| | | GCAGGACAGCAAGGACTCCACCTACAGCCTGAGCAGCA |
| | | CCCTGACCCTGAGCAAGGCCGACTACGAGAAGCACAAG |
| | | GTGTACGCCTGTGAGGTGACCCACCAGGGCCTGTCCAG |
| | | CCCCGTGACCAAGAGCTTCAACAGGGGCGAGTGC |
| BAP050-Clone I HC | | |
| SEQ ID NO: 736 | HCDR1 | AATTACGGGATGAAC |
| (Kabat) | | |
| SEQ ID NO: 737 | HCDR1 | AACTACGGCATGAAC |
| (Kabat) | | |
| SEQ ID NO: 738 | HCDR2 | TGGATTAACACCGACACCGGGGAGCCTACCTACGCGGA |
| (Kabat) | | CGATTTCAAGGGA |
| SEQ ID NO: 739 | HCDR2 | TGGATCAACACCGACACCGGCGAGCCTACCTACGCCGA |
| (Kabat) | | CGACTTCAAGGGC |
| SEQ ID NO: 740 | HCDR3 | AACCCGCCCTACTACTACGGAACCAACAACGCCGAAGC |
| (Kabat) | | CATGGACTAC |
| SEQ ID NO: 741 | HCDR3 | AACCCCCCTTACTACTACGGCACCAACAACGCCGAGGC |
| (Kabat) | | CATGGACTAT |
| SEQ ID NO: 742 | HCDR1 | GGATTCACCCTCACCAATTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGC |
| (Chothia) | | CATGGACTAC |
| SEQ ID NO: 741 | HCDR3 | AACCCCCCTTACTACTACGGCACCAACAACGCCGAGGC |
| (Chothia) | | CATGGACTAT |
| BAP050-Clone I LC | | |
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| SEQ ID NO: 750 | LCDR3 | CAGCAGTACTATAACCTGCCCTGGACC |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |
| BAP050-Clone J HC | | |
| SEQ ID NO: 758 | HCDR1 | AACTACGGGATGAAC |
| (Kabat) | | |
| SEQ ID NO: 737 | HCDR1 | AACTACGGCATGAAC |
| (Kabat) | | |
| SEQ ID NO: 759 | HCDR2 | TGGATTAACACCGACACCGGCGAGCCTACCTACGCCGA |
| (Kabat) | | CGACTTTAAGGGC |
| SEQ ID NO: 739 | HCDR2 | TGGATCAACACCGACACCGGCGAGCCTACCTACGCCGA |
| (Kabat) | | CGACTTCAAGGGC |
| SEQ ID NO: 760 | HCDR3 | AACCCCCCCTACTACTACGGCACTAACAACGCCGAGGC |
| (Kabat) | | TATGGACTAC |
| SEQ ID NO: 741 | HCDR3 | AACCCCCCTTACTACTACGGCACCAACAACGCCGAGGC |
| (Kabat) | | CATGGACTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGC |
| (Chothia) | | TATGGACTAC |
| SEQ ID NO: 741 | HCDR3 | AACCCCCCTTACTACTACGGCACCAACAACGCCGAGGC |
| (Chothia) | | CATGGACTAT |
| BAP050-Clone J LC | | |
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| SEQ ID NO: 750 | LCDR3 | CAGCAGTACTATAACCTGCCCTGGACC |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

### Other Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 9.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 9. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 9
Amino acid sequences of other exemplary
anti-LAG-3 antibody molecules

| | | |
|---|---|---|
| BMS-986016 | | |
| SEQ ID NO: | Heavy | QVQLQQWGAGLLKPSETLSLTCAVYGGSFSDYYWNWIRQPP |
| 762 | chain | GKGLEWIGEINHRGSTNSNPSLKSRVTLSLDTSKNQFSLKLRS |
| | | VTAADTAVYYCAFGYSDYEYNWFDPWGQGTLVTVSSASTKG |
| | | PSVFPLAPCSRSTSESTAALGCLVKDYFPEPVTVSWNSGALTS |
| | | GVHTFPAVLQSSGLYSLSSVVTVPSSSLGTKTYTCNVDHKPSN |
| | | TKVDKRVESKYGPPCPPCPAPEFLGGPSVFLFPPKPKDTLMIS |
| | | RTPEVTCVVVDVSQEDPEVQFNWYVDGVEVHNAKTKPREEQ |
| | | FNSTYRVVSVLTVLHQDWLNGKEYKCKVSNKGLPSSIEKTISK |
| | | AKGQPREPQVYTLPPSQEEMTKNQVSLTCLVKGFYPSDIAVE |
| | | WESNGQPENNYKTTPPVLDSDGSFFLYSRLTVDKSRWQEGN |
| | | VFSCSVMHEALHNHYTQKSLSLSLGK |
| SEQ ID NO: | Light | EIVLTQSPATLSLSPGERATLSCRASQSISSYLAWYQQKPGQA |
| 763 | chain | PRLLIYDASNRATGIPARFSGSGSGTDFTLTISSLEPEDFAVYY |
| | | CQQRSNWPLTFGQGTNLEIKRTVAAPSVFIFPPSDEQLKSGTA |
| | | SVVCLLNNFYPREAKVQWKVDNALQSGNSQESVTEQDSKDST |
| | | YSLSSTLTLSKADYEKHKVYACEVTHQGLSSPVTKSFNRGEC |
| IMP731 | | |
| SEQ ID NO: | Heavy | QVQLKESGPGLVAPSQSLSITCTVSGFSLTAYGVNWVRQPPG |
| 764 | chain | KGLEWLGMIWDDGSTDYNSALKSRLSISKDNSKSQVFLKMNS |
| | | LQTDDTARYYCAREGDVAFDYWGQGTTLTVSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHTCPPCPAPELLGGPSVFLFPPKPKDTLMISR |
| | | TPEVTCVVVDVSHEDPEVKFNWYVDGVEVHNAKTKPREEQYN |
| | | STYRWSVLTVLHQDWLNGKEYKCKVSNKALPAPIEKTISKAK |
| | | GQPREPQVYTLPPSRDELTKNQVSLTCLVKGFYPSDIAVEWES |
| | | NGQPENNYKTTPPVLDSDGSFFLYSKLTVDKSRWQQGNVFSC |
| | | SVMHEALHNHYTQKSLSLSPGK |
| SEQ ID NO: | Light | DIVMTQSPSSLAVSVGQKVTMSCKSSQSLLNGSNQKNYLAWY |
| 765 | chain | QQKPGQSPKLLVYFASTRDSGVPDRFIGSGSGTDFTLTISSVQ |
| | | AEDLADYFCLQHFGTPPTFGGGTKLEIKRTVAAPSVFIFPPSDE |
| | | QLKSGTASVVCLLNNFYPREAKVQWKVDNALQSGNSQESVTE |
| | | QDSKDSTYSLSSTLTLSKADYEKHKVYACEVTHQGLSSPVTKS |
| | | FNRGEC |

### TIM-3 Inhibitors

In certain embodiments, the inhibitor of an immune checkpoint molecule is an inhibitor of TIM-3. In some embodiments, the antibody conjugate of the present invention is administered in combination with a TIM-3 inhibitor. In some embodiments, the TIM-3 inhibitor is MGB453 (Novartis) or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 10 (e.g., from the heavy and light chain variable region sequences of ABTIM3-hum11 or ABTIM3-hum03 disclosed in Table 10), or encoded by a nucleotide sequence shown in Table 10. In some embodiments, the CDRs are according to the Kabat definition (e.g., as set out in Table 10). In some embodiments, the CDRs are according to the Chothia definition (e.g., as set out in Table 10). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 10, or encoded by a nucleotide sequence shown in Table 10.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 10. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 10.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823.

In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 10
Amino acid and nucleotide sequences of exemplary
anti-TIM-3 antibody molecules

| | | |
|---|---|---|
| ABTIM3-hum11 | | |
| SEQ ID NO: 801 | HCDR1 | SYNMH |
| (Kabat) | | |
| SEQ ID NO: 802 | HCDR2 | DIYPGNGDTSYNQKFKG |
| (Kabat) | | |
| SEQ ID NO: 803 | HCDR3 | VGGAFPMDY |
| (Kabat) | | |
| SEQ ID NO: 804 | HCDR1 | GYTFTSY |
| (Chothia) | | |
| SEQ ID NO: 805 | HCDR2 | YPGNGD |
| (Chothia) | | |
| SEQ ID NO: 803 | HCDR3 | VGGAFPMDY |
| (Chothia) | | |
| SEQ ID NO: 806 | VH | QVQLVQSGAEVKKPGSSVKVSCKASGYTFTSYNMHWVR |
| | | QAPGQGLEWMGDIYPGNGDTSYNQKFKGRVTITADKST |
| | | STVYMELSSLRSEDTAVYYCARVGGAFPMDYWGQGTTV |
| | | TVSS |
| SEQ ID NO: 807 | DNA VH | CAGGTGCAGCTGGTGCAGTCAGGCGCCGAAGTGAAG |
| | | AAACCCGGCTCTAGCGTGAAAGTTTCTTGTAAAGCTAG |
| | | TGGCTACACCTTCACTAGCTATAATATGCACTGGGTTC |
| | | GCCAGGCCCCAGGGCAAGGCCTCGAGTGGATGGGCG |
| | | ATATCTACCCCGGGAACGGCGACACTAGTTATAATCAG |
| | | AAGTTTAAGGGTAGAGTCACTATCACCGCCGATAAGTC |
| | | TACTAGCACCGTCTATATGGAACTGAGTTCCCTGAGGT |
| | | CTGAGGACACCGCCGTCTACTACTGCGCTAGAGTGGG |
| | | CGGAGCCTTCCCTATGGACTACTGGGGTCAAGGCACT |
| | | ACCGTGACCGTGTCTAGC |
| SEQ ID NO: 808 | Heavy | QVQLVQSGAEVKKPGSSVKVSCKASGYTFTSYNMHWVR |
| | chain | QAPGQGLEWMGDIYPGNGDTSYNQKFKGRVTITADKST |
| | | STVYMELSSLRSEDTAVYYCARVGGAFPMDYWGQGTTV |
| | | TVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAPE |
| | | FLGGPSVFLFPPKPKDTLMISRTPEVTCVVVDVSQEDPEV |
| | | QFNWYVDGVEVHNAKTKPREEQFNSTYRVVSVLTVLHQ |
| | | DWLNGKEYKCKVSNKGLPSSIEKTISKAKGQPREPQVYT |
| | | LPPSQEEMTKNQVSLTCLVKGFYPSDIAVEWESNGQPEN |
| | | NYKTTPPVLDSDGSFFLYSRLTVDKSRWQEGNVFSCSV |
| | | MHEALHNHYTQKSLSLSLG |
| SEQ ID NO: 809 | DNA | CAGGTGCAGCTGGTGCAGTCAGGCGCCGAAGTGAAG |
| | heavy | AAACCCGGCTCTAGCGTGAAAGTTTCTTGTAAAGCTAG |
| | chain | TGGCTACACCTTCACTAGCTATAATATGCACTGGGTTC |
| | | GCCAGGCCCCAGGGCAAGGCCTCGAGTGGATGGGCG |
| | | ATATCTACCCCGGGAACGGCGACACTAGTTATAATCAG |
| | | AAGTTTAAGGGTAGAGTCACTATCACCGCCGATAAGTC |
| | | TACTAGCACCGTCTATATGGAACTGAGTTCCCTGAGGT |
| | | CTGAGGACACCGCCGTCTACTACTGCGCTAGAGTGGG |
| | | CGGAGCCTTCCCTATGGACTACTGGGGTCAAGGCACT |
| | | ACCGTGACCGTGTCTAGCGCTAGCACTAAGGGCCCGT |
| | | CCGTGTTCCCCCTGGCACCTTGTAGCCGGAGCACTAG |
| | | CGAATCCACCGCTGCCCTCGGCTGCCTGGTCAAGGAT |
| | | TACTTCCCGGAGCCCGTGACCGTGTCCTGGAACAGCG |
| | | GAGCCCTGACCTCCGGAGTGCACACCTTCCCCGCTGT |
| | | GCTGCAGAGCTCCGGGCTGTACTCGCTGTCGTCGGTG |
| | | GTCACGGTGCCTTCATCTAGCCTGGGTACCAAGACCT |
| | | ACACTTGCAACGTGGACCACAAGCCTTCCAACACTAAG |
| | | GTGGACAAGCGCGTCGAATCGAAGTACGGCCCACCGT |
| | | GCCCGCCTTGTCCCGCGCCGGAGTTCCTCGGCGGTC |
| | | CCTCGGTCTTTCTGTTCCCACCGAAGCCCAAGGACAC |
| | | TTTGATGATTTCCCGCACCCCTGAAGTGACATGCGTGG |
| | | TCGTGGACGTGTCACAGGAAGATCCGGAGGTGCAGTT |
| | | CAATTGGTACGTGGATGGCGTCGAGGTGCACAACGCC |
| | | AAAACCAAGCCGAGGGAGGAGCAGTTCAACTCCACTT |
| | | ACCGCGTCGTGTCCGTGCTGACGGTGCTGCATCAGGA |
| | | CTGGCTGAACGGGAAGGAGTACAAGTGCAAAGTGTCC |
| | | AACAAGGGACTTCCTAGCTCAATCGAAAAGACCATCTC |
| | | GAAAGCCAAGGGACAGCCCCGGGAACCCCAAGTGTAT |
| | | ACCCTGCCACCGAGCCAGGAAGAAATGACTAAGAACC |
| | | AAGTCTCATTGACTTGCCTTGTGAAGGGCTTCTACCCA |
| | | TCGGATATCGCCGTGGAATGGGAGTCCAACGGCCAGC |
| | | CGGAAAACAACTACAAGACCACCCCTCCGGTGCTGGA |
| | | CTCAGACGGATCCTTCTTCCTCTACTCGCGGCTGACC |
| | | GTGGATAAGAGCAGATGGCAGGAGGGAAATGTGTTCA |
| | | GCTGTTCTGTGATGCATGAAGCCCTGCACAACCACTAC |
| | | ACTCAGAAGTCCCTGTCCCTCTCCCTGGGA |
| SEQ ID NO: 810 | LCDR1 | RASESVEYYGTSLMQ |
| (Kabat) | | |
| SEQ ID NO: 811 | LCDR2 | AASNVES |
| (Kabat) | | |
| SEQ ID NO: 812 | LCDR3 | QQSRKDPST |
| (Kabat) | | |
| SEQ ID NO: 813 | LCDR1 | SESVEYYGTSL |
| (Chothia) | | |
| SEQ ID NO: 814 | LCDR2 | AAS |
| (Chothia) | | |
| SEQ ID NO: 815 | LCDR3 | SRKDPS |
| (Chothia) | | |
| SEQ ID NO: 816 | VL | AIQLTQSPSSLSASVGDRVTITCRASESVEYYGTSLMQW |
| | | YQQKPGKAPKLLIYAASNVESGVPSRFSGSGSGTDFTLTI |
| | | SSLQPEDFATYFCQQSRKDPSTFGGGTKVEIK |
| SEQ ID NO: 817 | DNA VL | GCTATTCAGCTGACTCAGTCACCTAGTAGCCTGAGCG |
| | | CTAGTGTGGGCGATAGAGTGACTATCACCTGTAGAGC |
| | | TAGTGAATCAGTCGAGTACTACGGCACTAGCCTGATG |
| | | CAGTGGTATCAGCAGAAGCCCGGGAAAGCCCCTAAGC |
| | | TGCTGATCTACGCCGCCTCTAACGTGGAATCAGGCGT |
| | | GCCCTCTAGGTTTAGCGGTAGCGGTAGTGGCACCGAC |
| | | TTCACCCTGACTATCTCTAGCCTGCAGCCCGAGGACTT |
| | | CGCTACCTACTTCTGTCAGCAGTCTAGGAAGGACCCTA |
| | | GCACCTTCGGCGGAGGCACTAAGGTCGAGATTAAG |
| SEQ ID NO: 818 | Light | AIQLTQSPSSLSASVGDRVTITCRASESVEYYGTSLMQW |
| | chain | YQQKPGKAPKLLIYAASNVESGVPSRFSGSGSGTDFTLTI |
| | | SSLQPEDFATYFCQQSRKDPSTFGGGTKVEIKRTVAAPS |
| | | VFIFPPSDEQLKSGTASVVCLLNNFYPREAKVQWKVDNA |
| | | LQSGNSQESVTEQDSKDSTYSLSSTLTLSKADYEKHKVY |
| | | ACEVTHQGLSSPVTKSFNRGEC |
| SEQ ID NO: 819 | DNA | GCTATTCAGCTGACTCAGTCACCTAGTAGCCTGAGCG |
| | light | CTAGTGTGGGCGATAGAGTGACTATCACCTGTAGAGC |
| | chain | TAGTGAATCAGTCGAGTACTACGGCACTAGCCTGATG |
| | | CAGTGGTATCAGCAGAAGCCCGGGAAAGCCCCTAAGC |
| | | TGCTGATCTACGCCGCCTCTAACGTGGAATCAGGCGT |
| | | GCCCTCTAGGTTTAGCGGTAGCGGTAGTGGCACCGAC |
| | | TTCACCCTGACTATCTCTAGCCTGCAGCCCGAGGACTT |
| | | CGCTACCTACTTCTGTCAGCAGTCTAGGAAGGACCCTA |
| | | GCACCTTCGGCGGAGGCACTAAGGTCGAGATTAAGCG |
| | | TACGGTGGCCGCTCCCAGCGTGTTCATCTTCCCCCCC |
| | | AGCGACGAGCAGCTGAAGAGCGGCACCGCCAGCGTG |
| | | GTGTGCCTGCTGAACAACTTCTACCCCCGGGAGGCCA |
| | | AGGTGCAGTGGAAGGTGGACAACGCCCTGCAGAGCG |
| | | GCAACAGCCAGGAGAGCGTCACCGAGCAGGACAGCA |
| | | AGGACTCCACCTACAGCCTGAGCAGCACCCTGACCCT |
| | | GAGCAAGGCCGACTACGAGAAGCATAAGGTGTACGCC |
| | | TGCGAGGTGACCCACCAGGGCCTGTCCAGCCCCGTG |
| | | ACCAAGAGCTTCAACAGGGGCGAGTGC |
| ABTIM3-hum03 | | |
| SEQ ID NO: 801 | HCDR1 | SYNMH |
| (Kabat) | | |
| SEQ ID NO: 820 | HCDR2 | DIYPGQGDTSYNQKFKG |
| (Kabat) | | |
| SEQ ID NO: 803 | HCDR3 | VGGAFPMDY |
| (Kabat) | | |
| SEQ ID NO: 804 | HCDR1 | GYTFTSY |
| (Chothia) | | |
| SEQ ID NO: 821 | HCDR2 | YPGQGD |
| (Chothia) | | |
| SEQ ID NO: 803 | HCDR3 | VGGAFPMDY |
| (Chothia) | | |
| SEQ ID NO: 822 | VH | QVQLVQSGAEVKKPGASVKVSCKASGYTFTSYNMHWVR |
| | | QAPGQGLEWIGDIYPGQGDTSYNQKFKGRATMTADKST |
| | | STVYMELSSLRSEDTAVYYCARVGGAFPMDYWGQGTLV |
| | | TVSS |
| SEQ ID NO: 823 | DNA VH | CAGGTGCAGCTGGTGCAGTCAGGCGCCGAAGTGAAG |
| | | AAACCCGGCGCTAGTGTGAAAGTTAGCTGTAAAGCTA |
| | | GTGGCTATACTTTCACTTCTTATAATATGCACTGGGTC |
| | | CGCCAGGCCCCAGGTCAAGGCCTCGAGTGGATCGGC |
| | | GATATCTACCCCGGTCAAGGCGACACTTCCTATAATCA |
| | | GAAGTTTAAGGGTAGAGCTACTATGACCGCCGATAAGT |
| | | CTACTTCTACCGTCTATATGGAACTGAGTTCCCTGAGG |
| | | TCTGAGGACACCGCCGTCTACTACTGCGCTAGAGTGG |
| | | GCGGAGCCTTCCCAATGGACTACTGGGGTCAAGGCAC |
| | | CCTGGTCACCGTGTCTAGC |
| SEQ ID NO: 824 | Heavy | QVQLVQSGAEVKKPGASVKVSCKASGYTFTSYNMHWVR |
| | chain | QAPGQGLEWIGDIYPGQGDTSYNQKFKGRATMTADKST |
| | | STVYMELSSLRSEDTAVYYCARVGGAFPMDYWGQGTLV |
| | | TVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAPE |
| | | FLGGPSVFLFPPKPKDTLMISRTPEVTCVVVDVSQEDPEV |
| | | QFNWYVDGVEVHNAKTKPREEQFNSTYRVVSVLTVLHQ |
| | | DWLNGKEYKCKVSNKGLPSSIEKTISKAKGQPREPQVYT |
| | | LPPSQEEMTKNQVSLTCLVKGFYPSDIAVEWESNGQPEN |
| | | NYKTTPPVLDSDGSFFLYSRLTVDKSRWQEGNVFSCSV |
| | | MHEALHNHYTQKSLSLSLG |
| SEQ ID NO: 825 | DNA | CAGGTGCAGCTGGTGCAGTCAGGCGCCGAAGTGAAG |
| | heavy | AAACCCGGCGCTAGTGTGAAAGTTAGCTGTAAAGCTA |
| | chain | GTGGCTATACTTTCACTTCTTATAATATGCACTGGGTC |
| | | CGCCAGGCCCCAGGTCAAGGCCTCGAGTGGATCGGC |
| | | GATATCTACCCCGGTCAAGGCGACACTTCCTATAATCA |
| | | GAAGTTTAAGGGTAGAGCTACTATGACCGCCGATAAGT |
| | | CTACTTCTACCGTCTATATGGAACTGAGTTCCCTGAGG |
| | | TCTGAGGACACCGCCGTCTACTACTGCGCTAGAGTGG |
| | | GCGGAGCCTTCCCAATGGACTACTGGGGTCAAGGCAC |
| | | CCTGGTCACCGTGTCTAGCGCTAGCACTAAGGGCCCG |
| | | TCCGTGTTCCCCCTGGCACCTTGTAGCCGGAGCACTA |
| | | GCGAATCCACCGCTGCCCTCGGCTGCCTGGTCAAGGA |
| | | TTACTTCCCGGAGCCCGTGACCGTGTCCTGGAACAGC |
| | | GGAGCCCTGACCTCCGGAGTGCACACCTTCCCCGCTG |
| | | TGCTGCAGAGCTCCGGGCTGTACTCGCTGTCGTCGGT |
| | | GGTCACGGTGCCTTCATCTAGCCTGGGTACCAAGACC |
| | | TACACTTGCAACGTGGACCACAAGCCTTCCAACACTAA |
| | | GGTGGACAAGCGCGTCGAATCGAAGTACGGCCCACC |
| | | GTGCCCGCCTTGTCCCGCGCCGGAGTTCCTCGGCGG |
| | | TCCCTCGGTCTTTCTGTTCCCACCGAAGCCCAAGGAC |
| | | ACTTTGATGATTTCCCGCACCCCTGAAGTGACATGCGT |
| | | GGTCGTGGACGTGTCACAGGAAGATCCGGAGGTGCA |
| | | GTTCAATTGGTACGTGGATGGCGTCGAGGTGCACAAC |
| | | GCCAAAACCAAGCCGAGGGAGGAGCAGTTCAACTCCA |
| | | CTTACCGCGTCGTGTCCGTGCTGACGGTGCTGCATCA |
| | | GGACTGGCTGAACGGGAAGGAGTACAAGTGCAAAGTG |
| | | TCCAACAAGGGACTTCCTAGCTCAATCGAAAAGACCAT |
| | | CTCGAAAGCCAAGGGACAGCCCCGGGAACCCCAAGT |
| | | GTATACCCTGCCACCGAGCCAGGAAGAAATGACTAAG |
| | | AACCAAGTCTCATTGACTTGCCTTGTGAAGGGCTTCTA |
| | | CCCATCGGATATCGCCGTGGAATGGGAGTCCAACGGC |
| | | CAGCCGGAAAACAACTACAAGACCACCCCTCCGGTGC |
| | | TGGACTCAGACGGATCCTTCTTCCTCTACTCGCGGCT |
| | | GACCGTGGATAAGAGCAGATGGCAGGAGGGAAATGTG |
| | | TTCAGCTGTTCTGTGATGCATGAAGCCCTGCACAACCA |
| | | CTACACTCAGAAGTCCCTGTCCCTCTCCCTGGGA |
| SEQ ID NO: 810 | LCDR1 | RASESVEYYGTSLMQ |
| (Kabat) | | |
| SEQ ID NO: 811 | LCDR2 | AASNVES |
| (Kabat) | | |
| SEQ ID NO: 812 | LCDR3 | QQSRKDPST |
| (Kabat) | | |
| SEQ ID NO: 813 | LCDR1 | SESVEYYGTSL |
| (Chothia) | | |
| SEQ ID NO: 814 | LCDR2 | AAS |
| (Chothia) | | |
| SEQ ID NO: 815 | LCDR3 | SRKDPS |
| (Chothia) | | |
| SEQ ID NO: 826 | VL | DIVLTQSPDSLAVSLGERATINCRASESVEYYGTSLMQW |
| | | YQQKPGQPPKLLIYAASNVESGVPDRFSGSGSGTDFTLTI |
| | | SSLQAEDVAVYYCQQSRKDPSTFGGGTKVEIK |
| SEQ ID NO: 827 | DNA VL | GATATCGTCCTGACTCAGTCACCCGATAGCCTGGCCG |
| | | TCAGCCTGGGCGAGCGGGCTACTATTAACTGTAGAGC |
| | | TAGTGAATCAGTCGAGTACTACGGCACTAGCCTGATG |
| | | CAGTGGTATCAGCAGAAGCCCGGTCAACCCCCTAAGC |
| | | TGCTGATCTACGCCGCCTCTAACGTGGAATCAGGCGT |
| | | GCCCGATAGGTTTAGCGGTAGCGGTAGTGGCACCGAC |
| | | TTCACCCTGACTATTAGTAGCCTGCAGGCCGAGGACG |
| | | TGGCCGTCTACTACTGTCAGCAGTCTAGGAAGGACCC |
| | | TAGCACCTTCGGCGGAGGCACTAAGGTCGAGATTAAG |
| SEQ ID NO: 828 | Light | DIVLTQSPDSLAVSLGERATINCRASESVEYYGTSLMQW |
| | chain | YQQKPGQPPKLLIYAASNVESGVPDRFSGSGSGTDFTLTI |
| | | SSLQAEDVAVYYCQQSRKDPSTFGGGTKVEIKRTVAAPS |
| | | VFIFPPSDEQLKSGTASVVCLLNNFYPREAKVQWKVDNA |
| | | LQSGNSQESVTEQDSKDSTYSLSSTLTLSKADYEKHKVY |
| | | ACEVTHQGLSSPVTKSFNRGEC |
| SEQ ID NO: 829 | DNA | GATATCGTCCTGACTCAGTCACCCGATAGCCTGGCCG |
| | light | TCAGCCTGGGCGAGCGGGCTACTATTAACTGTAGAGC |
| | chain | TAGTGAATCAGTCGAGTACTACGGCACTAGCCTGATG |
| | | CAGTGGTATCAGCAGAAGCCCGGTCAACCCCCTAAGC |
| | | TGCTGATCTACGCCGCCTCTAACGTGGAATCAGGCGT |
| | | GCCCGATAGGTTTAGCGGTAGCGGTAGTGGCACCGAC |
| | | TTCACCCTGACTATTAGTAGCCTGCAGGCCGAGGACG |
| | | TGGCCGTCTACTACTGTCAGCAGTCTAGGAAGGACCC |
| | | TAGCACCTTCGGCGGAGGCACTAAGGTCGAGATTAAG |
| | | CGTACGGTGGCCGCTCCCAGCGTGTTCATCTTCCCCC |
| | | CCAGCGACGAGCAGCTGAAGAGCGGCACCGCCAGCG |
| | | TGGTGTGCCTGCTGAACAACTTCTACCCCCGGGAGGC |
| | | CAAGGTGCAGTGGAAGGTGGACAACGCCCTGCAGAG |
| | | CGGCAACAGCCAGGAGAGCGTCACCGAGCAGGACAG |
| | | CAAGGACTCCACCTACAGCCTGAGCAGCACCCTGACC |
| | | CTGAGCAAGGCCGACTACGAGAAGCATAAGGTGTACG |
| | | CCTGCGAGGTGACCCACCAGGGCCTGTCCAGCCCCG |
| | | TGACCAAGAGCTTCAACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 11. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 11
Amino acid sequences of other exemplary anti-TIM-3 antibody molecules

| | | |
|---|---|---|
| APE5137 | | |
| SEQ ID NO: 830 | VH | EVQLLESGGGLVQPGGSLRLSCAAASGFTFSSYDMSWVRQAPGK |
| | | GLDWVSTISGGGTYTYYQDSVKGRFTISRDNSKNTLYLQMNSLRAE |
| | | DTAVYYCASMDYWGQGTTVTVSSA |
| SEQ ID NO: 831 | VL | DIQMTQSPSSLSASVGDRVTITCRASQSIRRYLNWYHQKPGKAPKL |
| | | LIYGASTLQSGVPSRFSGSGSGTDFTLTISSLQPEDFAVYYCQQSH |
| | | SAPLTFGGGTKVEIKR |
| APE5121 | | |
| SEQ ID NO: 832 | VH | EVQVLESGGGLVQPGGSLRLYCVASGFTFSGSYAMSWVRQAPGK |
| | | GLEWVSAISGSGGSTYYADSVKGRFTISRDNSKNTLYLQMNSLRAE |
| | | DTAVYYCAKKYYVGPADYWGQGTLVTVSSG |
| SEQ ID NO: 833 | VL | DIVMTQSPDSLAVSLGERATINCKSSQSVLYSSNNKNYLAWYQHKP |
| | | GQPPKLLIYWASTRESGVPDRFSGSGSGTDFTLTISSLQAEDVAVY |
| | | YCQQYYSSPLTFGGGTKIEVK |

Cytokines

In yet another embodiment, the present invention provides a method of treating cancer by administering to a subject in need thereof antibody conjugate of the present invention in combination with one or more cytokines, including but not limited to, interferon, IL2, IL15, IL7, or IL21. In certain embodiments, antibody conjugate is administered in combination with an IL-15/IL-15Ra complex. In some embodiments, the IL-15/IL-15Ra complex is selected from NIZ985 (Novartis), ATL-803 (Altor) or CYP0150 (Cytune).

Exemplary IL-15/IL-15Ra Complexes

In one embodiment, the cytokine is IL-15 complexed with a soluble form of IL-15 receptor alpha (IL-15Ra). The IL-15/IL-15Ra complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 922 in Table 12 or an amino acid sequence at least 85%, 90%, 95%, or 99% identical or higher to SEQ ID NO: 922, and the soluble form of human IL-15Ra comprises an amino acid sequence of SEQ ID NO:923 in Table 12, or an amino acid sequence at least 85%, 90%, 95%, or 99% identical or higher to SEQ ID NO: 923, as described in WO 2014/066527, incorporated by reference in its entirety. The molecules described herein can be made by vectors, host cells, and methods described in WO 2007084342, incorporated by reference in its entirety.

TABLE 12
Amino acid and nucleotide sequences of
exemplary IL-15/IL-15Ra complexes
NIZ985

| | | |
|---|---|---|
| SEQ ID NO: | Human IL-15 | NVWNVISDLKKIEDLIQSMHIDATLYTESDVHPSCKVTAMKCF |
| 922 | | LLELQVISLESGDASIHDTVENLIILANNSLSSNGNVTESGCKE |
| | | CEELEEKNIKEFLQSFVHIVQMFINTS |
| SEQ ID NO: | Human | ITCPPPMSVEHADIVWKSYSLYSRERYICNSGFKRKAGTSSLT |
| 923 | Soluble IL- | ECVLNKATNVAHWTTPSLKCIRDPALVHQRPAPPSTVTTAGV |
| | 15Ra | TPQPESLSPSGKEPAASSPSSNNTAATTAAIVPGSQLMPSKS |
| | | PSTGTTEISSHESSHGTPSQTTAKNWELTASASHQPPGVYPQ |
| | | G |

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is described in WO 2008/143794, incorporated by reference in its entirety. In one embodiment, the IL-15/IL-15Ra Fc fusion protein comprises the sequences as disclosed in Table 13.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is described in WO 2007/04606 and WO 2012/175222, incorporated by reference in their entirety. In one embodiment, the IL-15/IL-15Ra sushi domain fusion comprises the sequences as disclosed in Table 13.

TABLE 13
Amino acid sequences of other exemplary IL-15/IL-15Ra complexes
ALT-803

| | | |
|---|---|---|
| SEQ ID | IL-15N72D | NWVNVISDLKKIEDLIQSMHIDATLYTESDVHPSCKVTAMKCF |
| NO: 924 | | LLELQVISLESGDASIHDTVENLIILANDSLSSNGNVTESGCKE |
| | | CEELEEKNIKEFLQSFVHIVQMFINTS |
| SEQ ID | IL-15RaSu/ | ITCPPPMSVEHADIWVKSYSLYSRERYICNSGFKRKAGTSSLT |
| NO: 925 | Fc | ECVLNKATNVAHWTTPSLKCIREPKSCDKTHTCPPCPAPELL |
| | | GGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSRDELTKN |
| | | QVSLTCLVKGFYPSDIAVEWESNGQPENNYKTTPPVLDSDGS |
| | | FFLYSKLTVDKSRWQQGNVFSCSVMHEALHNHYTQKSLSLS |
| | | PGK |

IL-15/IL-15Ra sushi domain fusion (CYP0150)

| | | |
|---|---|---|
| SEQ ID | Human IL-15 | NWVNVISDLKKIEDLIQSMHIDATLYTESDVHPSCKVTAMKCF |
| NO: 926 | | LLELQVISLESGDASIHDTVENLIILANNSLSSNGNVTESGCKE |
| | | CEELEXKNIKEFLQSFVHIVQMFINTS |
| | | Where X is E or K |
| SEQ ID | Human IL- | ITCPPPMSVEHADIWVKSYSLYSRERYICNSGFKRKAGTSSLT |
| NO: 927 | 15Ra sushi | ECVLNKATNVAHWTTPSLKCIRDPALVHQRPAPP |
| | and hinge | |
| | domains | |

In yet another embodiment, the present invention provides a method of treating cancer by administering to a subject in need thereof the anti-DC-SIGN antibodies, antibody fragments, antibody conjugates, or fusion proteins of the present invention in combination with one or more agonists of toll like receptors (TLRs, e.g., TLR7, TLR8, TLR9). In some embodiments, the antibody conjugate of the present invention can be used in combination with a TLR7 agonist or a TLR7 agonist conjugate.

In another embodiment, the present invention provides a method of treating cancer by administering to a subject in need thereof the anti-DC-SIGN antibodies, antibody fragments, antibody conjugates, or fusion proteins of the present invention in combination with one or more angiogenesis inhibitors, e.g., Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TK1258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); or Aflibercept (Eylea®).

In another embodiment, the present invention provides a method of treating cancer by administering to a subject in need thereof the anti-DC-SIGN antibodies, antibody fragments, antibody conjugates, or fusion proteins of the present invention in combination with one or more heat shock protein inhibitors, e.g., Tanespimycin (17-allylamino-17-demethoxygeldanamycin, also known as KOS-953 and 17-AAG, available from SIGMA, and described in U.S. Pat. No. 4,261,989); Retaspimycin (IPI504), Ganetespib (STA-9090); [6-Chloro-9-(4-methoxy-3,5-dimethylpyridin-2-ylmethyl)-9H-purin-2-yl]amine (BIIB021 or CNF2024, CAS 848695-25-0); trans-4-[[2-(Aminocarbonyl)-5-[4,5,6,7-tetrahydro-6,6-dimethyl-4-oxo-3-(trifluoromethyl)-1H-indazol-1-yl]phenyl]amino]cyclohexyl glycine ester (SNX5422 or PF04929113, CAS 908115-27-5); 5-[2,4-Dihydroxy-5-(1-methylethyl)phenyl]-N-ethyl-4-[4-(4-morpholinylmethyl)phenyl]-3-Isoxazolecarboxamide (AUY922, CAS 747412-49-3); or 17-Dimethylaminoethylamino-17-demethoxygeldanamycin (17-DMAG).

In another embodiment, the present invention provides a method of treating cancer by administering to a subject in need thereof the anti-DC-SIGN antibodies, antibody fragments, antibody conjugates, or fusion proteins of the present invention in combination with one or more HDAC inhibitors or other epigenetic modifiers. Exemplary HDAC inhibitors include, but not limited to, Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl] (Cyl-1); Cyclo[(αS,2S)-α-amino-η-oxo-2-oxiraneoctanoyl-O-methyl-D-tyrosyl-L-isoleucyl-(2S)-2-piperidinecarbonyl] (Cyl-2); Cyclic[L-alanyl-D-alanyl-(2S)-η-oxo-L-α-aminooxiraneoctanoyl-D-prolyl] (HC-toxin); Cyclo[(αS,2S)-α-amino-η-oxo-2-oxiraneoctanoyl-D-phenylalanyl-L-leucyl-(2S)-2-piperidinecarbonyl] (WF-3161); Chlamydocin ((S)-Cyclic(2-methylalanyl-L-phenylalanyl-D-prolyl-η-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); Depudecin (4,5:8,9-dianhydro-1,2,6,7,11-pentadeoxy-D-threo-D-ido-Undeca-1,6-dienitol); 4-(Acetylamino)-N-(2-aminophenyl)-benzamide (also known as CI-994); N1-(2-Aminophenyl)-N8-phenyl-octanediamide (also known as BML-210); 4-(Dimethylamino)-N-(7-(hydroxyamino)-7-oxoheptyl)benzamide (also known as M344); (E)-3-(4-(((2-(1H-indol-3-yl)ethyl)(2-hydroxyethyl)amino)-methyl)phenyl)-N-hydroxyacrylamide; Panobinostat (Farydak®); Mocetinostat, and Belinostat (also known as PXD101, Beleodaq®, or (2E)-N-Hydroxy-3-[3-(phenylsulfamoyl)phenyl]prop-2-enamide), or chidamide (also known as CS055 or HBI-8000, (E)-N-(2-amino-5-fluorophenyl)-4-((3-(pyridin-3-yl)acrylamido)methyl)benzamide). Other epigenetic modifiers include but not limited to inhibitors of EZH2 (enhancer of zeste homolog 2), EED (embryonic ectoderm development), or LSD1 (lysine-specific histone demethylase 1A or KDM1A).

In yet another embodiment, the present invention provides a method of treating cancer by administering to a subject in need thereof the anti-DC-SIGN antibodies, antibody fragments, antibody conjugates, or fusion proteins of the present invention in combination with one or more inhibitors of indoleamine-pyrrole 2,3-dioxygenase (IDO), for example, Indoximod (also known as NLG-8189), α-Cyclohexyl-5H-imidazo[5,1-a]isoindole-5-ethanol (also known as NLG919), or (4E)-4-[(3-Chloro-4-fluoroanilino)-nitrosomethylidene]-1,2,5-oxadiazol-3-amine (also known as INCB024360).

In yet another embodiment, the present invention provides a method of treating cancer by administering to a subject in need thereof the anti-DC-SIGN antibodies, antibody fragments, antibody conjugates, or fusion proteins of the present invention in combination with one or more agents that control or treat cytokine release syndrome (CRS). Therapies for CRS include but not are limited to, IL-6 inhibitor or IL-6 receptor (IL-6R) inhibitors (e.g., tocilizumab or siltuximab), bazedoxifene, sgp130 blockers, vasoactive medications, corticosteroids, immunosuppressive agents, histamine H₂ receptor antagonists, anti-pyretics, analgesics (e.g., acetaminophen), and mechanical ventilation. Exemplary therapies for CRS are described in International Application WO2014011984, which is hereby incorporated by reference.

Tocilizumab is a humanized, immunoglobulin G1kappa anti-human IL-6R monoclonal antibody. Tocilizumab blocks binding of IL-6 to soluble and membrane bound IL-6 receptors (IL-6Rs) and thus inhibitors classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 (sgp130) or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

Exemplary vasoactive medications include but are not limited to angiotensin-11, endothelin-1, alpha adrenergic agonists, rostanoids, phosphodiesterase inhibitors, endothelin antagonists, inotropes (e.g., adrenaline, dobutamine, isoprenaline, ephedrine), vasopressors (e.g., noradrenaline, vasopressin, metaraminol, vasopressin, methylene blue), inodilators (e.g., milrinone, levosimendan), and dopamine.

Exemplary vasopressors include but are not limited to norepinephrine, dopamine, phenylephrine, epinephrine, and vasopressin. In some embodiments, a high-dose vasopressor includes one or more of the following: norpepinephrine monotherapy at ≥20 ug/min, dopamine monotherapy at ≥10 ug/kg/min, phenylephrine monotherapy at ≥200 ug/min, and/or epinephrine monotherapy at ≥10 ug/min. In some embodiments, if the subject is on vasopressin, a high-dose vasopressor includes vasopressin+norepinephrine equivalent of ≥10 ug/min, where the norepinephrine equivalent dose=[norepinephrine (ug/min)]+[dopamine (ug/kg/min)/2]+[epinephrine (ug/min)]+[phenylephrine (ug/min)/10]. In some embodiments, if the subject is on combination vasopressors (not vasopressin), a high-dose vasopressor includes norepinephrine equivalent of ≥20 ug/min, where the norepinephrine equivalent dose=[norepinephrine (ug/min)]+[dopamine (ug/kg/min)/2]+[epinephrine (ug/min)]+[phenylephrine (ug/min)/10]. See e.g., Id.

In some embodiments, a low-dose vasopressor is a vasopressor administered at a dose less than one or more of the doses listed above for high-dose vasopressors.

Exemplary corticosteroids include but are not limited to dexamethasone, hydrocortisone, and methylprednisolone. In embodiments, a dose of dexamethasone of 0.5 mg/kg is used. In embodiments, a maximum dose of dexamethasone of 10 mg/dose is used. In embodiments, a dose of methylprednisolone of 2 mg/kg/day is used.

Exemplary immunosuppressive agents include but are not limited to an inhibitor of TNFα or an inhibitor of IL-1. In embodiments, an inhibitor of TNFα comprises an anti-TNFα antibody, e.g., monoclonal antibody, e.g., infliximab. In embodiments, an inhibitor of TNFα comprises a soluble TNFα receptor (e.g., etanercept). In embodiments, an IL-1 or IL-1R inhibitor comprises anakinra.

Exemplary histamine H₂ receptor antagonists include but are not limited to cimetidine (Tagamet®), ranitidine (Zantac®), famotidine (Pepcid®) and nizatidine (Axid®).

Exemplary anti-pyretic and analgesic includes but is not limited to acetaminophen (Tylenol®), ibuprofen, and aspirin.

In some embodiments, the present invention provides a method of treating cancer by administering to a subject in need thereof the anti-DC-SIGN antibodies, antibody fragments, antibody conjugates, or fusion proteins of the present invention in combination with two or more of any of the above described inhibitors, activators, immunomodulators, agonists, or modifiers. For example, the antibody conjugate of the present invention can be used in combination with one or more checkpoint inhibitors and/or one or more immune activators.

In addition to the above therapeutic regimes, the patient may be subjected to surgical removal of cancer cells and/or radiation therapy.

### Pharmaceutical Compositions

To prepare pharmaceutical or sterile compositions including one or more antibodies, antibody fragments, antibody conjugates, or fusion proteins described herein, provided antibodies, antibody fragments, antibody conjugates, or fusion proteins can be mixed with a pharmaceutically acceptable carrier or excipient.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, N Y, 1993; Lieberman, et al. (eds.), Pharmaceutical Dosage Forms: Tablets, Marcel Dekker, N Y, 1990; Lieberman, et al. (eds.) Pharmaceutical Dosage Forms: Disperse Systems, Marcel Dekker, N Y, 1990; Weiner and Kotkoskie, Excipient Toxicity and Safety, Marcel Dekker, Inc., New York, N.Y., 2000).

In some embodiments, the pharmaceutical composition comprising the antibodies, antibody fragments, antibody conjugates, or fusion proteins of the present invention is a lyophilisate preparation. In certain embodiments a pharmaceutical composition comprising the antibody conjugate is a lyophilisate in a vial containing an antibody conjugate, histidine, sucrose, and polysorbate 20. In certain embodiments the pharmaceutical composition comprising the antibody, antibody conjugate, or fusion protein is a lyophilisate in a vial containing an antibody conjugate, sodium succinate, and polysorbate 20. In certain embodiments the pharmaceutical composition comprising the antibody conjugate is a lyophilisate in a vial containing an antibody conjugate, trehalose, citrate, and polysorbate 8. The lyophilisate can be reconstituted, e.g., with water, saline, for injection. In a specific embodiment, the solution comprises the antibody conjugate, histidine, sucrose, and polysorbate 20 at a pH of about 5.0. In another specific embodiment the solution comprises the antibody conjugate, sodium succinate, and polysorbate 20. In another specific embodiment, the solution comprises the antibody conjugate, trehalose dehydrate, citrate dehydrate, citric acid, and polysorbate 8 at a pH of about 6.6. For intravenous administration, the obtained solution will usually be further diluted into a carrier solution.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, U K, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising the antibody, antibody conjugate, or fusion protein of the invention can be provided by continuous infusion, or by doses at intervals of, e.g., one day, one week, or 1-7 times per week, once every other week, once every three weeks, once every four weeks, once every five weeks, once every six weeks, once every seven weeks, or once very eight weeks. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects.

For the antibodies, antibody fragments, antibody conjugates, or fusion proteins of the invention, the dosage administered to a patient may be 0.0001 mg/kg to 100 mg/kg of the patient's body weight. The dosage may be between 0.001 mg/kg and 50 mg/kg, 0.005 mg/kg and 20 mg/kg, 0.01 mg/kg and 20 mg/kg, 0.02 mg/kg and 10 mg/kg, 0.05 and 5 mg/kg, 0.1 mg/kg and 10 mg/kg, 0.1 mg/kg and 8 mg/kg, 0.1 mg/kg and 5 mg/kg, 0.1 mg/kg and 2 mg/kg, 0.1 mg/kg and 1 mg/kg of the patient's body weight. The dosage of the antibody conjugate may be calculated using the patient's weight in kilograms (kg) multiplied by the dose to be administered in mg/kg.

Doses of the antibodies, antibody fragments, antibody conjugates, or fusion proteins of the invention may be repeated and the administrations may be separated by less than 1 day, at least 1 day, 2 days, 3 days, 5 days, 10 days, 15 days, 30 days, 45 days, 2 months, 75 days, 3 months, 4 months, 5 months, or at least 6 months. In some embodiments, an antibody conjugate of the invention is administered twice weekly, once weekly, once every two weeks, once every three weeks, once every four weeks, or less frequently. In a specific embodiment, doses of the antibody conjugates of the invention are repeated every 2 weeks.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, U K, 2001).

The route of administration may be by, e.g., topical or cutaneous application, injection or infusion by subcutaneous, intravenous, intraperitoneal, intracerebral, intramuscular, intraocular, intraarterial, intracerebrospinal, intralesional administration, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

Examples of such additional ingredients are well-known in the art.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the antibodies, antibody fragments, antibody conjugates, or fusion proteins of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibody conjugates of the invention. The two or more therapies may be administered within one same patient visit.

In certain embodiments, the antibodies, antibody fragments, antibody conjugates, or fusion proteins of the invention can be formulated to ensure proper distribution in vivo. Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant Protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The invention provides protocols for the administration of pharmaceutical composition comprising antibodies, antibody fragments, antibody conjugates, or fusion proteins of the invention alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently.

The term “concurrently” is not limited to the administration of therapies (e.g., prophylactic or therapeutic agents) at exactly the same time, but rather it is meant that a pharmaceutical composition comprising antibodies, or fragments thereof, antibody conjugates, or fusion proteins of the invention are administered to a subject in a sequence and within a time interval such that the antibodies or antibody conjugates of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 5 minutes apart, less than 15 minutes apart, less than 30 minutes apart, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered within the same patient visit.

Prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

It is understood that the examples and embodiments described herein are for illustrative purposes only and that various modifications or changes in light thereof will be suggested to persons skilled in the art and are to be included within the spirit and purview of this application and scope of the appended claims.

### Examples

The invention is further described in the following examples, which are not intended to limit the scope of the invention described in the claims.

### Example 1

### Synthesis of 1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-1H-pyrrole-2,5-dione (C-1)

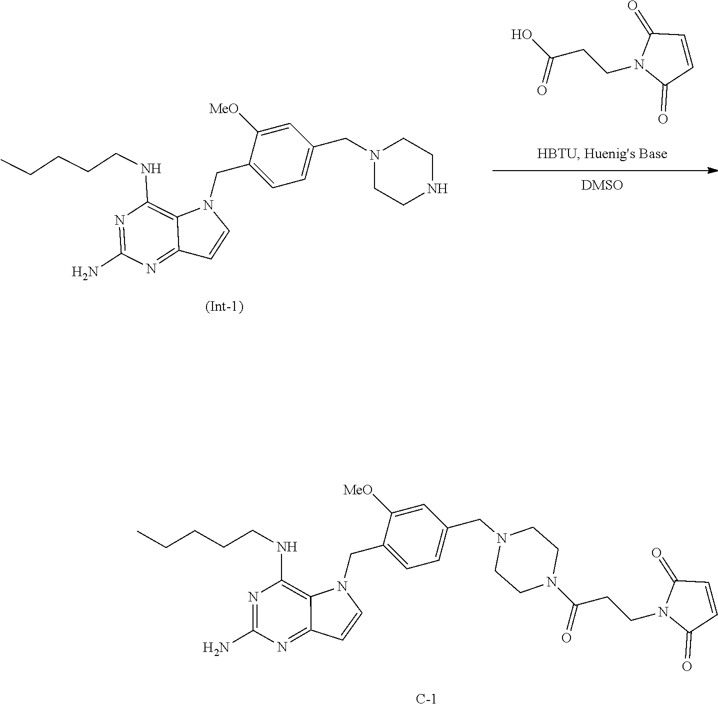

A round bottom flask was charged with 5-(2-methoxy-5-(piperazin-1-ylmethyl)benzyl-N4-pentyl-5H-pyrrolo[3,2-d]pyrimidine-2,4-diamine (Int-1, 1.0 equiv.), HBTU (1.2 equiv.), Huenig's base (3.0 equiv.), 3-(2,5-dioxo-2,5-dihydro-1H-pyrrol-1-yl)propanoic acid (1.2 equiv.) and DMSO (0.1 M). The reaction mixture was stirred at room temperature for 3 hours and then the crude reaction mixture was purified by RP-HPLC (0.035% TFA in ACN:0.05% TFA in H₂O, C18 column) to afford 1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-1H-pyrrole-2,5-dione (C-1) as a solid as the TFA salt: ¹H NMR (CDCl₃): δ 7.35 (d, 1H), 7.12 (d, 1H), 6.86 (d, 1H), 6.72 (s, 2H), 6.69 (d, 1H), 6.40 (s, 1H), 5.46 (t, 1H), 5.33 (s, 2H), 4.16 (s, 2H), 3.95 (s, 3H), 3.82 (m, 6H), 3.40 (m, 4H), 3.21 (m, 2H), 2.67 (m, 4H), 1.39 (m, 2H), 1.26 (m, 2H), 1.14 (m, 2H), 0.86 (t, 3H). LRMS [M+H]=589.3.

### Example 2

### Synthesis of (2R)-2-amino-3-((1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-2,5-dioxopyrrolidin-3-yl)thio)propanoic acid (C-2)

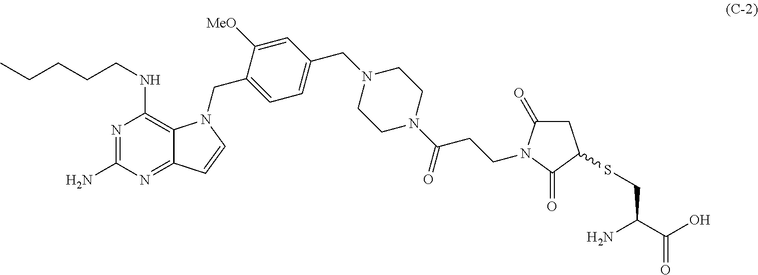 [SMILES: CCCCCNc1nc(N)nc2ccn(Cc3ccc(CN4CCN(C(=O)CCN5C(=O)CC(SC[C@H](N)C(=O)O)C5=O)CC4)cc3OC)c12]

A round bottom flask was charged with 1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-1H-pyrrole-2,5-dione (C-1, 1.0 equiv.) and dissolved in ACN-PBS buffer (1:2, 0.02 M). To this mixture was added L-cysteine (2.0 equiv.) dissolved in DPBS buffer (0.07 M). The reaction mixture was stirred at room temperature for 1 hour. The crude reaction mixture was purified by RP-HPLC (0.035% TFA in ACN:0.05% TFA in H₂O, C18 column) to afford (2R)-2-amino-3-((1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-2,5-dioxopyrrolidin-3-yl)thio)propanoic acid (C-2) as a solid as the TFA salt of a mixture of diastereomers: ¹H NMR (CD₃OD): δ 7.36 (d, 1H), 7.28 (d, 1H), 7.05 (d, 1H), 6.81 (d, 1H), 6.24 (d, 1H), 5.57 (s, 2H), 4.26 (m, 2H), 4.02 (m, 1H), 3.95 (s, 3H), 3.78 (m, 6H), 3.55 (m, 2H), 3.44 (m, 1H), 3.23 (m, 3H), 3.12 (m, 2H), 2.76 (m, 2H), 2.53 (m, 1H), 1.53 (m, 2H), 1.30 (m, 2H), 1.19 (m, 2H), 0.88 (t, 3H). LCMS [M+H]=710.3.

### Example 3

### Synthesis of (6R)-6-(2-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-2-oxoethyl)-5-oxothiomorpholine-3-carboxylic acid (C-3)

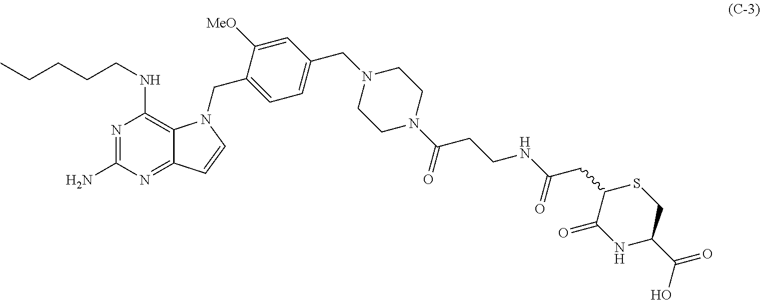

A round bottom flask was charged with (2R)-2-amino-3-((1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-2,5-dioxopyrrolidin-3-yl)thio)propanoic acid (C-1) and dissolved in PBS buffer (pH 7.5, 100 mM phosphate with 5 nM EDTA) and acetonitrile (1:1, 0.012 M). The reaction mixture was then stirred at 40° C. for 6 hours. At this point the crude reaction mixture was allowed to cool to room temperature and purified by RP-HPLC (0.5M NH₄OAc in ACN:10 mM NH₄OAc in H₂O, C18 column) to afford (6R)-6-(2-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-2-oxoethyl)-5-oxothiomorpholine-3-carboxylic acid (C-3) as a solid as a mixture of regio- and diastereomers. ¹H NMR (CD₃OD): δ 7.38 (d, 1H), 7.13 (s, 1H), 6.94 (d, 1H), 6.74 (d, 1H), 6.22 (d, 1H), 5.52 (s, 2H), 4.24 (m, 1H), 3.93 (s, 3H), 3.82 (m, 1H), 3.67 (s, 2H), 3.60 (m, 4H), 3.54 (t, 2H), 3.43 (m, 2H), 3.18 (m, 1H), 3.01 (m, 1H), 2.87 (m, 1H), 2.58 (m 7H), 1.50 (m, 2H), 1.29 (m, 2H), 1.17 (m, 2H), 0.88 (t, 3H). LCMS [M+H]=710.4.

### Example 4

### Synthesis of 3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid (C-4a) and 2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic Acid (C-4b)

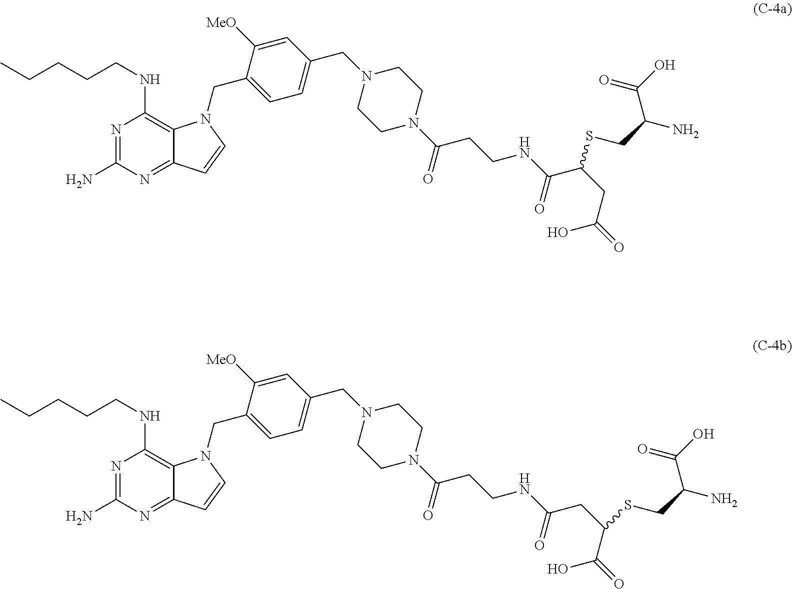 [SMILES: CCCCCNc1nc(N)nc2ccn(Cc3ccc(CN4CCN(C(=O)CCNC(=O)C(CC(=O)O)SC[C@H](N)C(=O)O)CC4)cc3OC)c12 ; CCCCCNc1nc(N)nc2ccn(Cc3ccc(CN4CCN(C(=O)CCNC(=O)CC(SC[C@H](N)C(=O)O)C(=O)O)CC4)cc3OC)c12]

A round bottom flask was charged with 1-(3-(4-(4-((2-amino-4-(pentylamino)-5H-pyrrolo[3,2-d]pyrimidin-5-yl)methyl)-3-methoxybenzyl)piperazin-1-yl)-3-oxopropyl)-1H-pyrrole-2,5-dione (C-1, 1.0 equiv.), L-cysteine (1.0 equiv.), and PBS:MeCN (2:1, 0.008 M). The reaction mixture was stirred at room temperature for 1 hour and then 1M NaOH (20.0 equiv.) was added to the reaction mixture. The reaction was then stirred an additional 3 hours, then the crude reaction mixture was purified by RP-HPLC (0.5 mM NH₄OAc in MeCN:10 mM NH₄OAc in H₂O, C18 column) to afford a mixture of 3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid (C-4a) and 2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic acid (C-4b), as their respective diasteromers (Compounds (C-4aSR), C-4aRR), (C-4bRR) and (C-4bRR) below) as a solid: ¹H NMR (DMSO): δ 7.88 (s, 1), 7.26 (s, 1H), 6.98 (s, 1H), 6.77 (d, 1H), 6.64 (s, 1H), 6.46 (s, 1H), 6.01 (s, 1H), 5.40 (s, 2H), 3.85 (s, 3H), 3.36 (m, 17H), 2.29 (m, 8H), 1.90 (s, 2H), 1.39 (m, 2H), 1.21 (m, 2H), 1.09 (m, 2H), 0.81 (t, 3H). LRMS [M+H]=728.4.

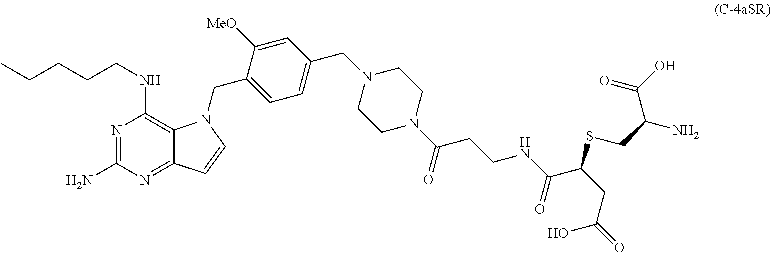 [SMILES: CCCCCNc1nc(N)nc2ccn(Cc3ccc(CN4CCN(C(=O)CCNC(=O)[C@H](CC(=O)O)SC[C@H](N)C(=O)O)CC4)cc3OC)c12]

(S)-3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic Acid (C-4aSR);

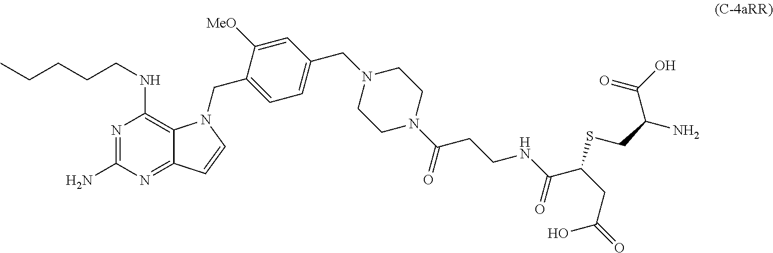 [SMILES: CCCCCNc1nc(N)nc2ccn(Cc3ccc(CN4CCN(C(=O)CCNC(=O)[C@@H](CC(=O)O)SC[C@H](N)C(=O)O)CC4)cc3OC)c12]

(R)-3-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic Acid (C-4aRR)

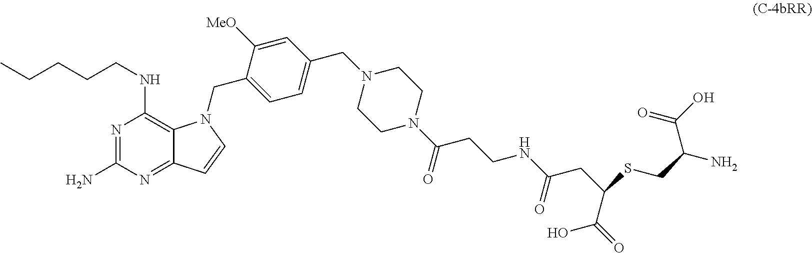 [SMILES: CCCCCNc1nc(N)nc2ccn(Cc3ccc(CN4CCN(C(=O)CCNC(=O)C[C@@H](SC[C@H](N)C(=O)O)C(=O)O)CC4)cc3OC)c12]

(R)-2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic Acid (C-4bRR)

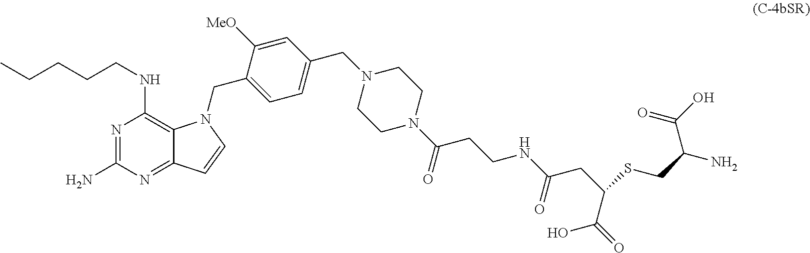 [SMILES: CCCCCNc1nc(N)nc2ccn(Cc3ccc(CN4CCN(C(=O)CCNC(=O)C[C@H](SC[C@H](N)C(=O)O)C(=O)O)CC4)cc3OC)c12]

(S)-2-(((R)-2-amino-2-carboxyethyl)thio)-4-((3-(4-(4-((2-amino-4-(pentylamino)-5H-pyrrolo[3,2-d]pyrimidin-5-yl)methyl)-3-methoxybenzyl)piperazin-1-yl)-3-oxopropyl)amino)-4-oxobutanoic Acid (C-4bSR).

### Example 5

### Synthesis of 1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-pyrrole-2,5-dione (C-5)

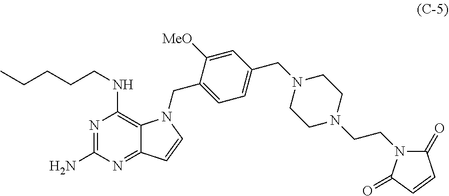 [SMILES: CCCCCNc1nc(N)nc2ccn(Cc3ccc(CN4CCN(CCN5C(=O)C=CC5=O)CC4)cc3OC)c12]

A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), 2-(2,5-dioxo-2,5-dihydro-1H-pyrrol-1-yl)acetaldehyde (4.0 equiv.), sodium cyanoborohydride (13.0 equiv.), and MeOH (0.04 M). The reaction mixture was stirred at room temperature for 1 hour and the crude reaction mixture was then purified by RP-HPLC (0.035% TFA in ACN:0.05% TFA in H₂O, C18 column) to afford 1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-pyrrole-2,5-dione (C-5) as a solid as the TFA salt: ¹H NMR (CDCl₃): δ 7.32 (d, 1H), 7.12 (d, 1H), 6.87 (d, 1H), 6.72 (s, 2H), 6.70 (d, 1H), 6.41 (d, 1H), 5.45 (t, 1H), 5.31 (s, 2H), 4.07 (s, 2H), 3.95 (s, 3H), 3.73 (t, 2H), 3.40 (m, 4H), 3.17 (m, 6H), 2.89 (m, 4H), 1.39 (m, 2H), 1.26 (m, 2H), 1.14 (m, 2H), 0.86 (t, 3H). LRMS [M+H]=561.3.

Note: 2-(2,5-dioxo-2,5-dihydro-1H-pyrrol-1-yl)acetaldehyde was prepared by adding 1-(2-hydroxyethyl)-1H-pyrrole-2,5-dione (1.0 equiv.), Dess-Martin periodinane (1.5 equiv.) and DCM (0.1 M) to a round bottom flask and stirring at room temperature for 2 hours. The reaction mixture was then filtered, the volatiles removed in vacuo and the product used without further purification.

### Example 6

### Synthesis of (2S)-2-amino-3-((1-(2-(4-(4-((2-amino-4-(pentylamino)-5H-pyrrolo[3,2-d]pyrimidin-5-yl)methyl)-3-methoxybenzyl)piperazin-1-yl)ethyl)-2,5-dioxopyrrolidin-3-yl)thio)propanoic acid (C-6)

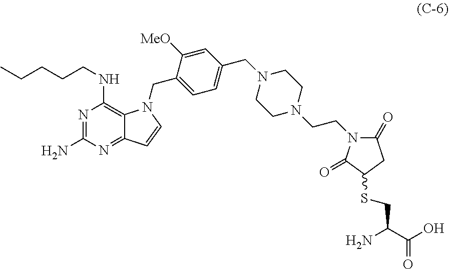 [SMILES: CCCCCNc1nc(N)nc2ccn(Cc3ccc(CN4CCN(CCN5C(=O)CC(SC[C@H](N)C(=O)O)C5=O)CC4)cc3OC)c12]

(2S)-2-amino-3-((1-(2-(4-(4-((2-amino-4-(pentylamino)-5H-pyrrolo[3,2-d]pyrimidin-5-yl)methyl)-3-methoxybenzyl)piperazin-1-yl)ethyl)-2,5-dioxopyrrolidin-3-yl)thio)propanoic acid (C-6) was prepared following a procedure similar to Example 2, except Compound (C-5) was used in place of Compound (C-1), to afford (2S)-2-amino-3-((1-(2-(4-(4-((2-amino-4-(pentylamino)-5H-pyrrolo[3,2-d]pyrimidin-5-yl)methyl)-3-methoxybenzyl)piperazin-1-yl)ethyl)-2,5-dioxopyrrolidin-3-yl)thio)propanoic acid (C-6) as a solid as the TFA salt of a mixture of diastereomers: ¹H NMR (CD₃OD): δ 7.36 (d, 1H), 7.21 (m, 1H), 7.02 (m, 1H), 6.78 (m, 1H), 6.23 (d, 1H), 5.56 (m, 2H), 4.21 (m, 1H), 4.09 (s, 1H), 4.03 (m, 1H), 3.95 (m, 3H), 3.75 (m, 2H), 3.54 (t, 2H), 3.43 (m, 1H), 3.34 (m, 1H), 3.22 (m, 2H), 3.03 (m, 6H), 2.84 (m, 2H), 2.63 (m, 1H), 1.52 (m, 2H), 1.30 (m, 2H), 1.18 (m, 2H), 0.88 (t, 3H). LCMS [M+H]=682.4.

### Example 7

### Synthesis of (6R)-6-(2-((2-(4-(4-((2-amino-4-(pentylamino)-5H-pyrrolo[3,2-d]pyrimidin-5-yl)methyl)-3-methoxybenzyl)piperazin-1-yl)ethyl)amino)-2-oxoethyl)-5-oxothiomorpholine-3-carboxylic Acid (C-7)

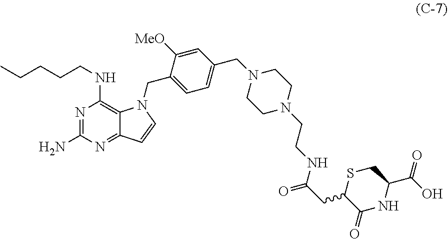

(6R)-6-(2-((2-(4-(4-((2-amino-4-(pentylamino)-5H-pyrrolo[3,2-d]pyrimidin-5-yl)methyl)-3-methoxybenzyl)piperazin-1-yl)ethyl)amino)-2-oxoethyl)-5-oxothiomorpholine-3-carboxylic acid (C-7) was prepared following a procedure similar to Example 3, except Compound (C-5) was used in place of Compound (C-1), to afford (6R)-6-(2-((2-(4-(4-((2-amino-4-(pentylamino)-5H-pyrrolo[3,2-d]pyrimidin-5-yl)methyl)-3-methoxybenzyl)piperazin-1-yl)ethyl)amino)-2-oxoethyl)-5-oxothiomorpholine-3-carboxylic Acid (C-7) as a solid as a mixture of regio- and diastereomers: ¹H NMR (CD₃OD): δ 7.37 (d, 1H), 7.10 (s, 1H), 6.91 (d, 1H), 6.72 (d, 1H), 6.22 (d, 1H), 5.51 (s, 2H), 4.13 (m, 1H), 3.92 (s, 3H), 3.88 (m, 1H), 3.58 (s, 2H), 3.52 (t, 2H), 3.40 (m, 2H), 3.16 (m, 1H), 2.99 (m, 1H), 2.86 (m, 1H), 2.67 (m 10H), 1.49 (m, 2H), 1.29 (m, 2H), 1.17 (m, 2H), 0.88 (t, 3H). LCMS [M+H]=682.3.

### Example 8

### Synthesis of 3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8a) and 2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8b)

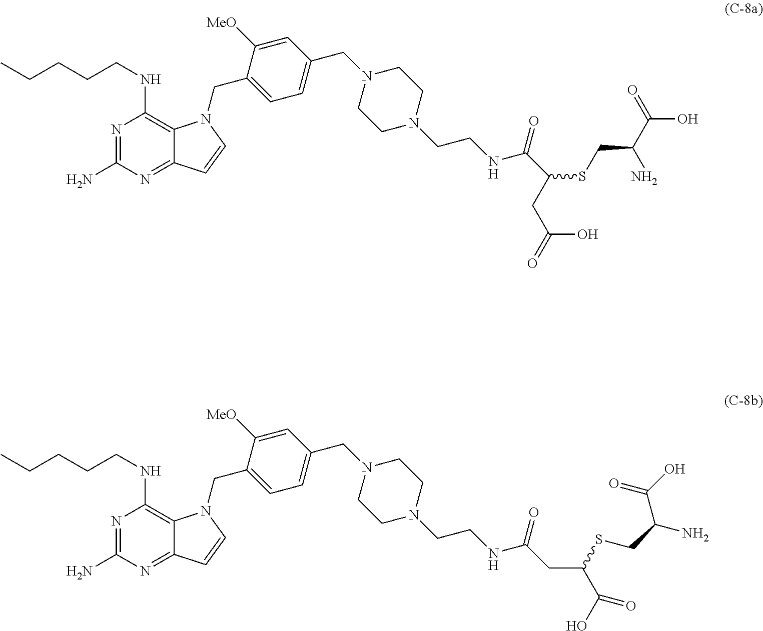 [SMILES: CCCCCNc1nc(N)nc2ccn(Cc3ccc(CN4CCN(CCNC(=O)C(CC(=O)O)SC[C@H](N)C(=O)O)CC4)cc3OC)c12 ; CCCCCNc1nc(N)nc2ccn(Cc3ccc(CN4CCN(CCNC(=O)CC(SC[C@H](N)C(=O)O)C(=O)O)CC4)cc3OC)c12]

3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8a) and 2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8b) were prepared following a procedure similar to Example 4, except Compound (C-5) was used in place of Compound (C-1), to give a mixture of Compounds (C-8a) and (C-8b), as their respective diasteromers (Compounds (C-8aSR), C-8aRR), (C-8bRR) and (C-8bRR) below), as a solid: ¹H NMR (DMSO): δ 7.81 (s, 1), 7.33 (s, 1H), 6.96 (s, 1H), 6.76 (d, 1H), 6.69 (s, 1H), 6.48 (s, 1H), 6.10 (s, 1H), 5.45 (s, 2H), 3.82 (s, 3H), 3.37 (m, 17H), 2.35 (m, 8H), 1.90 (s, 2H), 1.41 (m, 2H), 1.20 (m, 2H), 1.08 (m, 2H), 0.80 (t, 3H). LRMS [M+H]=700.4.

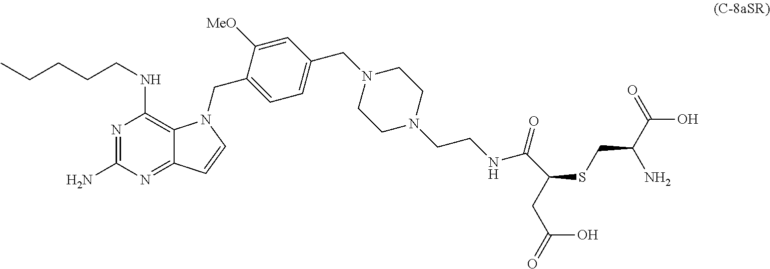 [SMILES: CCCCCNc1nc(N)nc2ccn(Cc3ccc(CN4CCN(CCNC(=O)[C@H](CC(=O)O)SC[C@H](N)C(=O)O)CC4)cc3OC)c12]

(S)-3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8aSR);

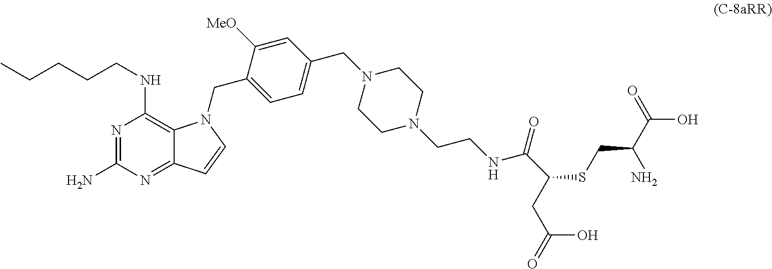 [SMILES: CCCCCNc1nc(N)nc2ccn(Cc3ccc(CN4CCN(CCNC(=O)[C@@H](CC(=O)O)SC[C@H](N)C(=O)O)CC4)cc3OC)c12]

(R)-3-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8aRR);

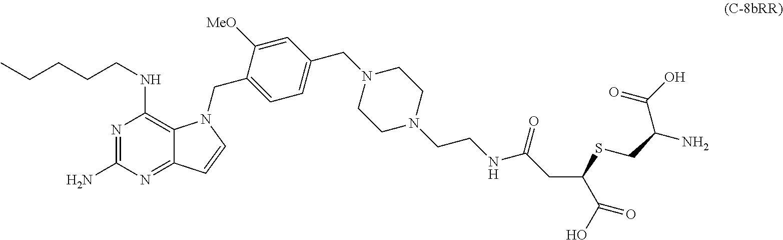 [SMILES: CCCCCNc1nc(N)nc2ccn(Cc3ccc(CN4CCN(CCNC(=O)C[C@@H](SC[C@H](N)C(=O)O)C(=O)O)CC4)cc3OC)c12]

(R)-2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8bRR);

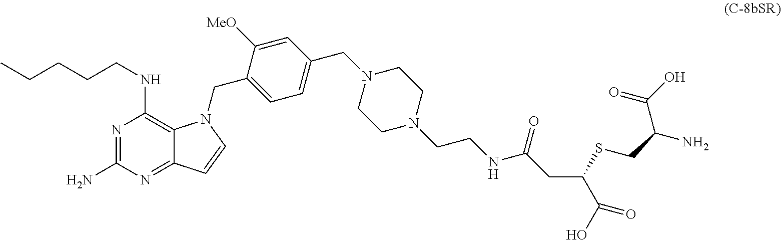 [SMILES: CCCCCNc1nc(N)nc2ccn(Cc3ccc(CN4CCN(CCNC(=O)C[C@H](SC[C@H](N)C(=O)O)C(=O)O)CC4)cc3OC)c12]

(S)-2-(((R)-2-amino-2-carboxyethyl)thio)-4-((2-(4-(4-((2-amino-4-(pentylamino)-5H-pyrrolo[3,2-d]pyrimidin-5-yl)methyl)-3-methoxybenzyl)piperazin-1-yl)ethyl)amino)-4-oxobutanoic Acid (C-8bSR).

### Example 9

### Synthesis of 1-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-1H-pyrrole-2,5-dione (C-9)

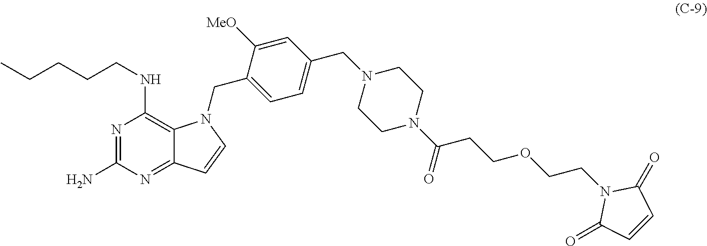 [SMILES: CCCCCNc1nc(N)nc2ccn(Cc3ccc(CN4CCN(C(=O)CCOCCN5C(=O)C=CC5=O)CC4)cc3OC)c12]

1-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-1H-pyrrole-2,5-dione (C-9) was prepared following a procedure similar to Example 1, except 3-(2-(2,5-dioxo-2,5-dihydro-1H-pyrrol-1-yl)ethoxy)propanoic acid was used in place of 3-(2,5-dioxo-2,5-dihydro-1H-pyrrol-1-yl)propanoic acid, to afford 1-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-1H-pyrrole-2,5-dione (C-9) as a solid as the TFA salt: ¹H NMR (CD₃OD): δ 7.37 (d, 1H), 7.27 (d, 1H), 7.06 (d, 1H), 6.82 (s, 2H), 6.81 (d, 1H), 6.24 (d, 1H), 5.58 (s, 2H), 4.38 (s, 2H), 3.96 (s, 3H), 3.86 (m, 4H), 3.67 (m, 4H), 3.56 (m, 4H), 3.24 (m, 4H), 2.61 (t, 2H), 1.53 (m, 2H), 1.31 (m, 2H), 1.20 (m, 2H), 0.88 (t, 3H). LCMS [M+H]=633.3.

### Example 10

### Synthesis of 3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic Acid (C-10a) and 2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic Acid (C-10b)

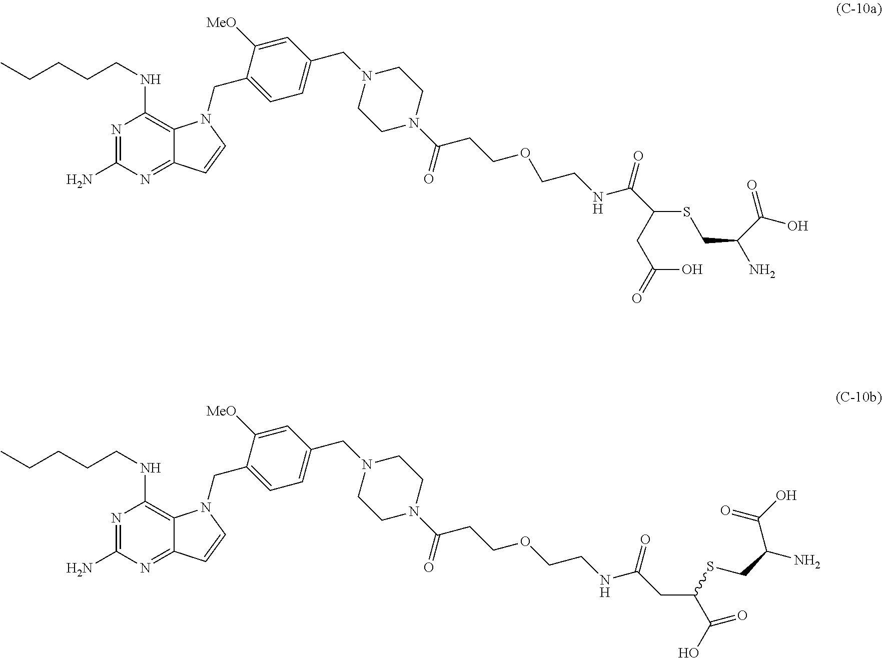 [SMILES: CCCCCNc1nc(N)nc2ccn(Cc3ccc(CN4CCN(C(=O)CCOCCNC(=O)C(CC(=O)O)SC[C@H](N)C(=O)O)CC4)cc3OC)c12 ; CCCCCNc1nc(N)nc2ccn(Cc3ccc(CN4CCN(C(=O)CCOCCNC(=O)CC(SC[C@H](N)C(=O)O)C(=O)O)CC4)cc3OC)c12]

3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid (C-10a) and 2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic acid (C-10b) were prepared following a procedure similar to Example 4, except Compound (C-9) was used in place of Compound (C-1), to afford a mixture of Compounds (C-10a) and (C-10b), as their respective diasteromers (Compounds (C-10aSR), C-10aRR), (C-10bRR) and (C-10bRR) below), as a solid as the TFA salt. The crude reaction mixture was purified by RP-HPLC (0.035% TFA in ACN:0.05% TFA in H₂O, C18 column): ¹H NMR (CD₃OD): δ 7.35 (d, 1H), 7.29 (d, 1H), 7.05 (d, 1H), 6.77 (m, 1H), 6.23 (s, 1H), 5.56 (s, 2H), 4.32 (m, 2H), 3.94 (s, 3H), 3.86 (m, 3H), 3.72 (m, 3H), 3.54 (m, 10H), 3.21 (m, 4H), 2.67 (m, 4H), 1.52 (m, 2H), 1.30 (m, 2H), 1.19 (m, 2H), 0.88 (t, 3H). LCMS [M+H]=772.4.

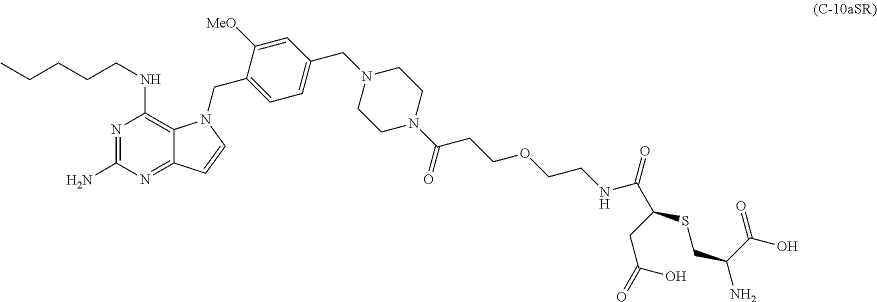 [SMILES: CCCCCNc1nc(N)nc2ccn(Cc3ccc(CN4CCN(C(=O)CCOCCNC(=O)[C@H](CC(=O)O)SC[C@H](N)C(=O)O)CC4)cc3OC)c12]

(S)-3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic Acid (C-10aSR);

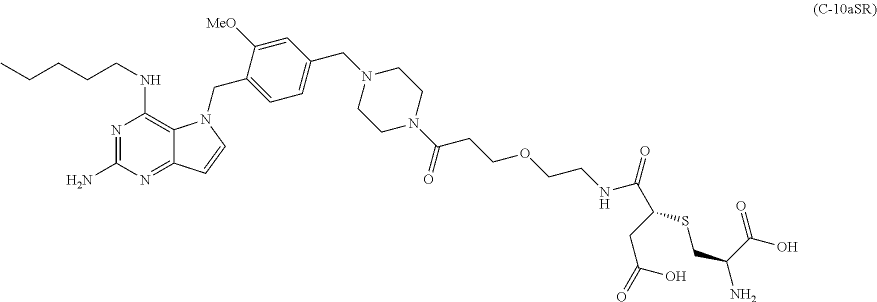 [SMILES: CCCCCNc1nc(N)nc2ccn(Cc3ccc(CN4CCN(C(=O)CCOCCNC(=O)[C@@H](CC(=O)O)SC[C@H](N)C(=O)O)CC4)cc3OC)c12]

(R)-3-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic Acid (C-10aRR);

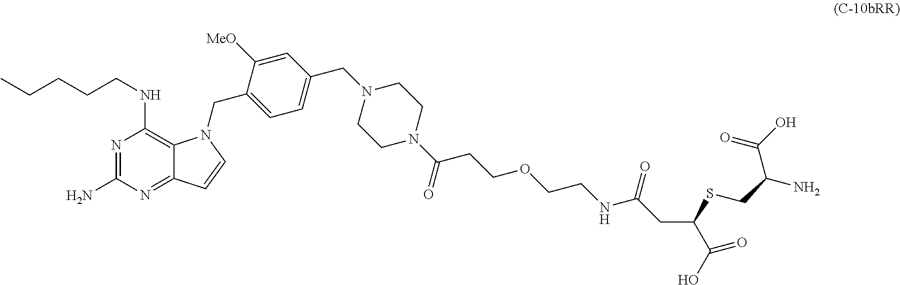 [SMILES: CCCCCNc1nc(N)nc2ccn(Cc3ccc(CN4CCN(C(=O)CCOCCNC(=O)C[C@@H](SC[C@H](N)C(=O)O)C(=O)O)CC4)cc3OC)c12]

(R)-2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic Acid (C-10bRR);

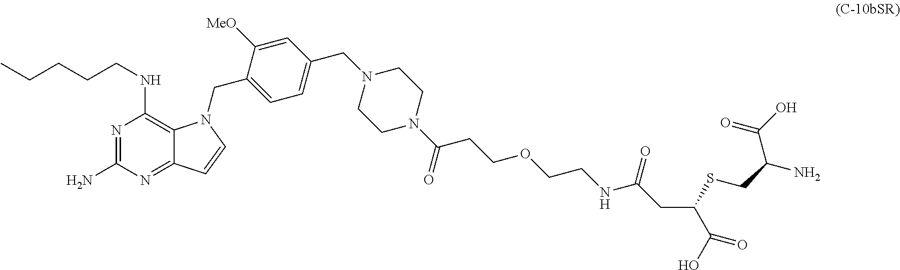 [SMILES: CCCCCNc1nc(N)nc2ccn(Cc3ccc(CN4CCN(C(=O)CCOCCNC(=O)C[C@H](SC[C@H](N)C(=O)O)C(=O)O)CC4)cc3OC)c12]

(S)-2-(((R)-2-amino-2-carboxyethyl)thio)-4-((2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)amino)-4-oxobutanoic Acid (C-10bSR).

### Example 11

### Synthesis of 1-(2-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethoxy)ethyl)-1H-pyrrole-2,5-dione (C-11)

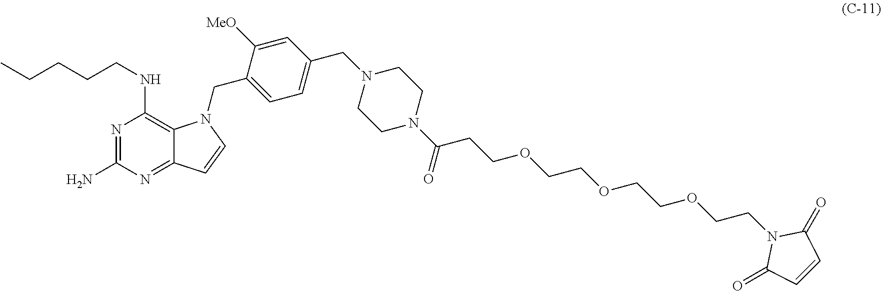 [SMILES: CCCCCNc1nc(N)nc2ccn(Cc3ccc(CN4CCN(C(=O)CCOCCOCCOCCN5C(=O)C=CC5=O)CC4)cc3OC)c12]

1-(2-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethoxy)ethyl)-1H-pyrrole-2,5-dione (C-11) was prepared following a procedure similar to Example 1, except 3-(2-(2-(2-(2,5-dioxo-2,5-dihydro-1H-pyrrol-1-yl)ethoxy)ethoxy)ethoxy)propanoic acid was used in place of 3-(2,5-dioxo-2,5-dihydro-1H-pyrrol-1-yl)propanoic acid, to afford 1-(2-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethoxy)ethyl)-1H-pyrrole-2,5-dione (C-11) as a solid as the TFA salt: ¹H NMR (CD₃OD): δ 7.37 (d, 1H), 7.26 (d, 1H), 7.05 (d, 1H), 6.82 (d, 1H), 6.80 (s, 2H), 6.24 (d, 1H), 5.58 (s, 2H), 4.32 (s, 2H), 3.96 (s, 3H), 3.74 (t, 2H), 3.64 (m, 2H), 3.58 (m, 12H), 3.64 (m, 4H), 3.20 (m, 4H), 2.68 (m, 2H), 1.53 (m, 2H), 1.32 (m, 2H), 1.20 (m, 2H), 0.88 (t, 3H). LCMS [M+H]=721.4.

### Example 12

### Synthesis of (2R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic Acid (C-12a) and (19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic Acid (C-12b)

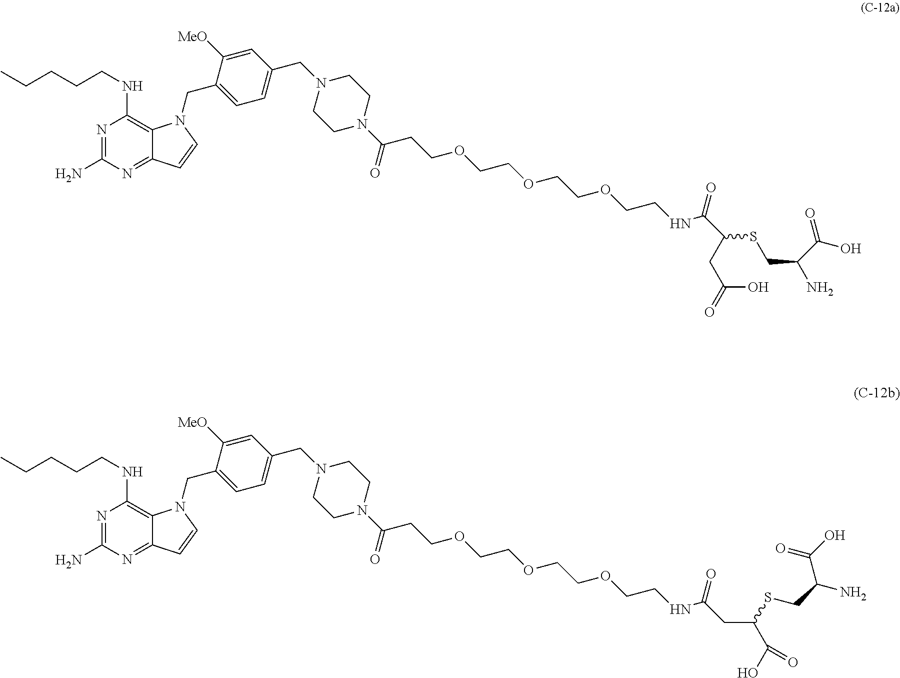 [SMILES: CCCCCNc1nc(N)nc2ccn(Cc3ccc(CN4CCN(C(=O)CCOCCOCCOCCNC(=O)C(CC(=O)O)SC[C@H](N)C(=O)O)CC4)cc3OC)c12 ; CCCCCNc1nc(N)nc2ccn(Cc3ccc(CN4CCN(C(=O)CCOCCOCCOCCNC(=O)CC(SC[C@H](N)C(=O)O)C(=O)O)CC4)cc3OC)c12]

(2R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic acid (C-12) and (19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic acid (C-12b) were prepared following a procedure similar to Example 4, except Compound (C-11) was used in place of Compound (C-1), to afford a mixture of Compounds (C-12a) and (C-12b), as their respective diasteromers (Compounds (C-12aSR), C-12aRR), (C-12bRR) and (C-12bRR) below), as a solid as the TFA salt. The crude reaction mixture was purified by RP-HPLC (0.035% TFA in ACN:0.05% TFA in H₂O, C18 column): ¹H NMR (CD₃OD): δ 7.36 (d, 1H), 7.31 (s, 1H), 7.06 (d, 1H), 6.79 (d, 1H), 6.24 (d, 1H), 5.57 (s, 2H), 4.34 (s, 2H), 4.23 (m, 1H), 3.96 (s, 3H), 3.86 (m, 4H), 3.76 (m, 4H), 3.58 (m, 14H), 3.27 (m, 4H), 3.22 (m, 2H), 2.84 (m, 1H), 2.71 (m, 2H), 1.53 (m, 2H), 1.31 (m, 2H), 1.19 (m, 2H), 0.88 (t, 3H). LCMS [M+H]=860.4.

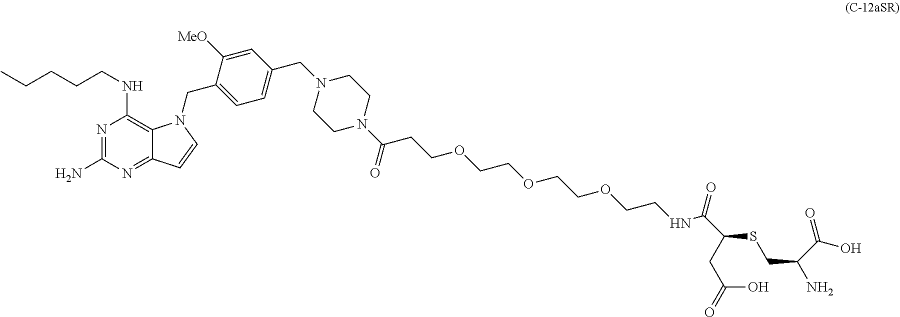 [SMILES: CCCCCNc1nc(N)nc2ccn(Cc3ccc(CN4CCN(C(=O)CCOCCOCCOCCNC(=O)[C@H](CC(=O)O)SC[C@H](N)C(=O)O)CC4)cc3OC)c12]

(2R,5S)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic Acid (C-12aSR);

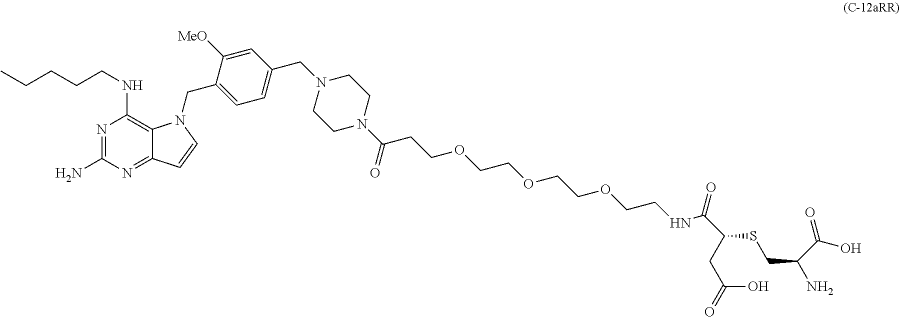 [SMILES: CCCCCNc1nc(N)nc2ccn(Cc3ccc(CN4CCN(C(=O)CCOCCOCCOCCNC(=O)[C@@H](CC(=O)O)SC[C@H](N)C(=O)O)CC4)cc3OC)c12]

(2R,5R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,19-dioxo-10,13,16-trioxa-4-thia-7-azanonadecan-1-oic Acid (C-12aRR);

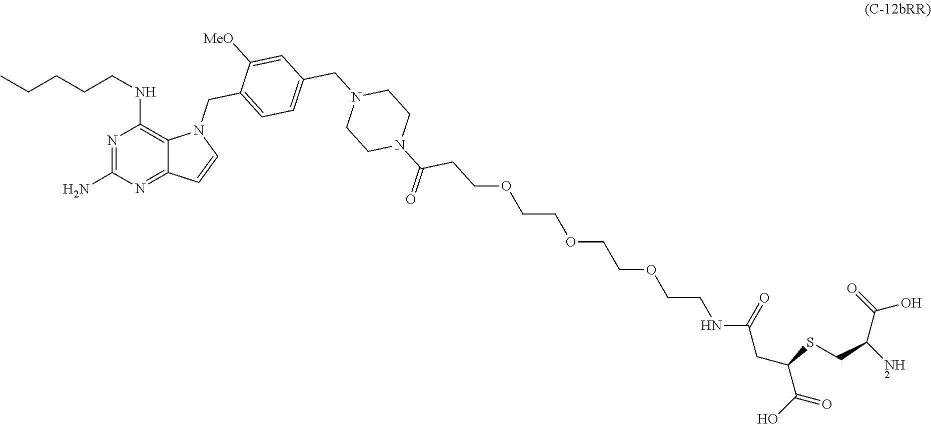 [SMILES: CCCCCNc1nc(N)nc2ccn(Cc3ccc(CN4CCN(C(=O)CCOCCOCCOCCNC(=O)C[C@@H](SC[C@H](N)C(=O)O)C(=O)O)CC4)cc3OC)c12]

(16R,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic Acid (C-12bRR);

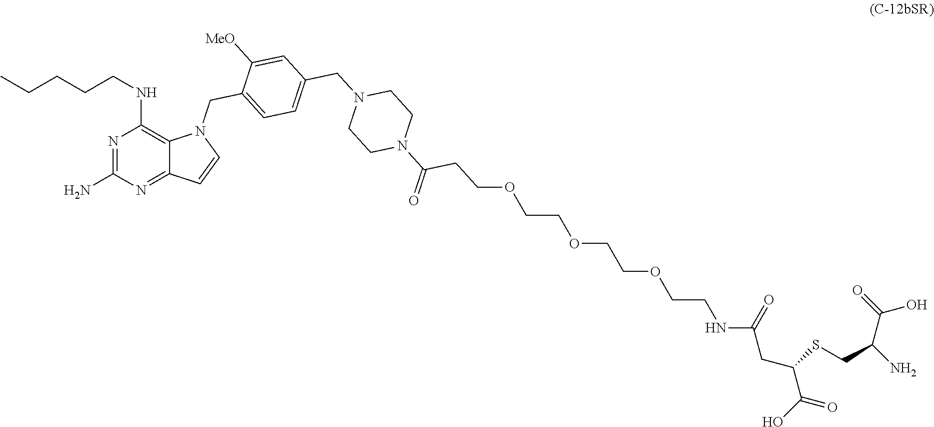 [SMILES: CCCCCNc1nc(N)nc2ccn(Cc3ccc(CN4CCN(C(=O)CCOCCOCCOCCNC(=O)C[C@H](SC[C@H](N)C(=O)O)C(=O)O)CC4)cc3OC)c12]

(16S,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,14-dioxo-4,7,10-trioxa-17-thia-13-azaicosan-20-oic Acid (C-12bSR).

### Example 13

### Synthesis of 1-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-1H-pyrrole-2,5-dione (C-13)

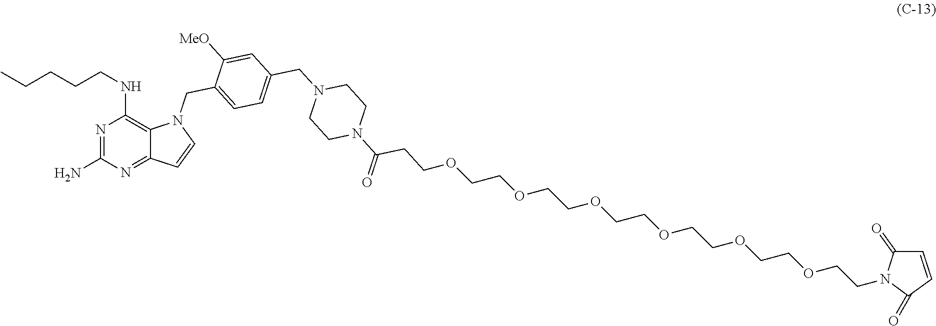 [SMILES: CCCCCNc1nc(N)nc2ccn(Cc3ccc(CN4CCN(C(=O)CCOCCOCCOCCOCCOCCOCCN5C(=O)C=CC5=O)CC4)cc3OC)c12]

1-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-1H-pyrrole-2,5-dione (C-13) was prepared following a procedure similar to example 1, except 1-(2,5-dioxo-2,5-dihydro-1H-pyrrol-1-yl)-3,6,9,12,15,18-hexaoxahenicosan-21-oic acid was used in place of 3-(2,5-dioxo-2,5-dihydro-1H-pyrrol-1-yl)propanoic acid, to afford 1-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-1H-pyrrole-2,5-dione (C-13) as a solid as the TFA salt: ¹H NMR (CD₃OD): δ 7.38 (d, 1H), 7.27 (d, 1H), 7.07 (d, 1H), 6.84 (d, 1H), 6.82 (s, 2H), 6.25 (d, 1H), 5.59 (s, 2H), 4.36 (s, 2H), 3.97 (s, 3H), 3.65 (m, 32H), 3.20 (m, 4H), 2.71 (m, 2H), 1.55 (m, 2H), 1.32 (m, 2H), 1.21 (m, 2H), 0.89 (t, 3H). LCMS [M+H]=853.5.

### Example 14

### Synthesis of (2R)-2-amino-28-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,28-dioxo-10,13,16,19,22,25-hexaoxa-4-thia-7-azaoctacosan-1-oic Acid (C-14a) and (28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic Acid (C-14b)

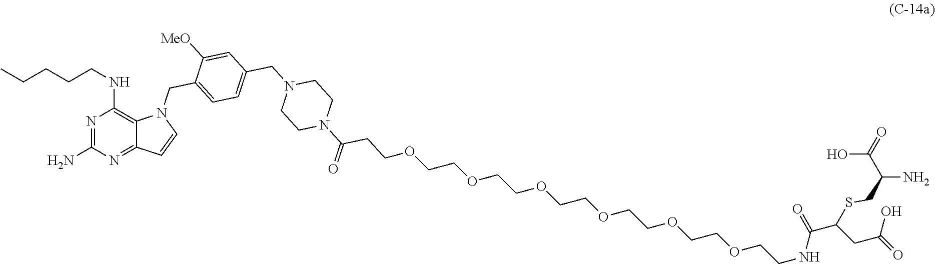 [SMILES: CCCCCNc1nc(N)nc2ccn(Cc3ccc(CN4CCN(C(=O)CCOCCOCCOCCOCCOCCOCCNC(=O)C(CC(=O)O)SC[C@H](N)C(=O)O)CC4)cc3OC)c12]

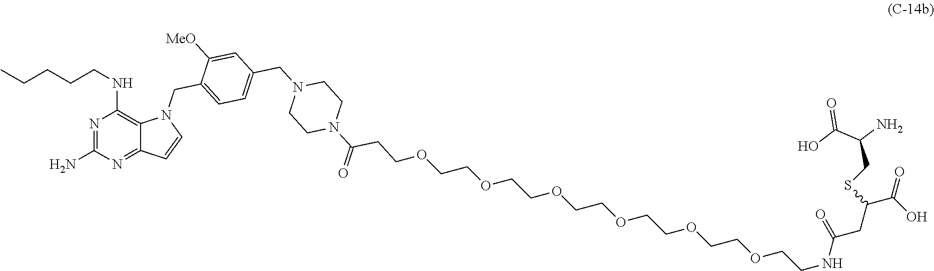 [SMILES: CCCCCNc1nc(N)nc2ccn(Cc3ccc(CN4CCN(C(=O)CCOCCOCCOCCOCCOCCOCCNC(=O)CC(SC[C@H](N)C(=O)O)C(=O)O)CC4)cc3OC)c12]

(2R)-2-amino-28-(4-(4-((2-amino-4-(pentylamino)-5H-pyrrolo[3,2-d]pyrimidin-5-yl)-methyl)-3-methoxybenzyl)piperazin-1-yl)-5-(carboxymethyl)-6,28-dioxo-10,13,16,19,22,25-hexaoxa-4-thia-7-azaoctacosan-1-oic acid (C-14a) and (28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic acid (C-14b) were prepared following a procedure similar to Example 4, except Compound (C-13) was used in place of Compound (C-1), to provide a mixture of Compounds (C-14a) and (C-14b), as their respective diasteromers (Compounds (C-14aSR), C-14aRR), (C-14bRR) and (C-14bRR) below), as a solid as the HCl salt (After RP-HPLC purification the product was dissolved in acetonitrile, treated with excess 2N HCl, and then lyophilized): ¹H NMR (CD₃OD): δ 7.47 (s, 1H), 7.39 (d, 1H), 7.13 (d, 1H), 6.82 (d, 1H), 6.25 (d, 1H), 5.58 (s, 2H), 4.38 (s, 2H), 4.32 (m, 1H), 4.00 (s, 3H), 3.77 (m, 4H), 3.76 (m, 4H), 3.64 (m, 28H), 3.55 (m, 5H), 3.31 (m, 4H), 3.12 (m, 1H), 2.86 (m, 1H), 2.72 (s, 2H), 2.62 (m, 1H), 1.54 (m, 2H), 1.31 (m, 2H), 1.20 (m, 2H), 0.89 (t, 3H). LCMS [M+H]=992.4.

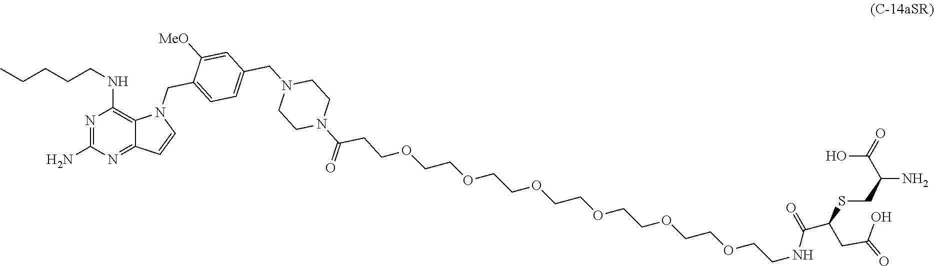 [SMILES: CCCCCNc1nc(N)nc2ccn(Cc3ccc(CN4CCN(C(=O)CCOCCOCCOCCOCCOCCOCCNC(=O)[C@H](CC(=O)O)SC[C@H](N)C(=O)O)CC4)cc3OC)c12]

(2R,5S)-2-amino-28-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,28-dioxo-10,13,16,19,22,25-hexaoxa-4-thia-7-azaoctacosan-1-oic Acid (C-14aSR);

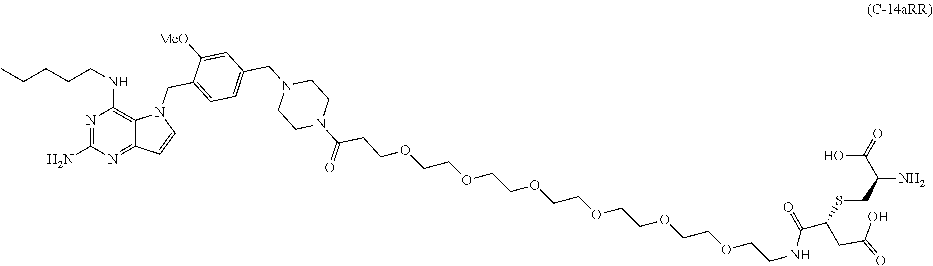 [SMILES: CCCCCNc1nc(N)nc2ccn(Cc3ccc(CN4CCN(C(=O)CCOCCOCCOCCOCCOCCOCCNC(=O)[C@@H](CC(=O)O)SC[C@H](N)C(=O)O)CC4)cc3OC)c12]

(2R,5R)-2-amino-28-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,28-dioxo-10,13,16,19,22,25-hexaoxa-4-thia-7-azaoctacosan-1-oic Acid (C-14aRR);

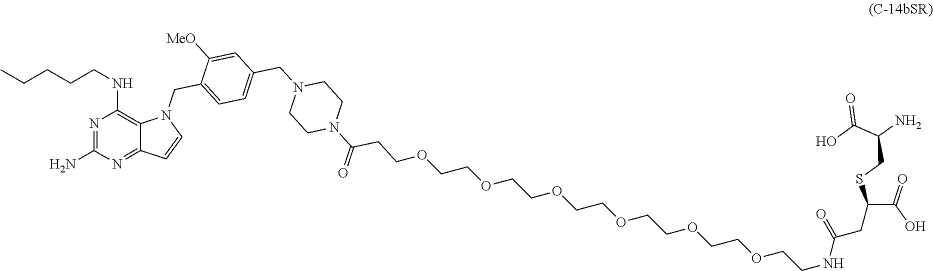 [SMILES: CCCCCNc1nc(N)nc2ccn(Cc3ccc(CN4CCN(C(=O)CCOCCOCCOCCOCCOCCOCCNC(=O)C[C@@H](SC[C@H](N)C(=O)O)C(=O)O)CC4)cc3OC)c12]

(25R,28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic Acid (C-14bRR);

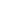

(25S,28R)-28-amino-1-(4-(4-((2-amino-4-(pentylamino)-5H-pyrrolo[3,2-d]pyrimidin-5-yl)methyl)-3-methoxybenzyl)piperazin-1-yl)-25-carboxy-1,23-dioxo-4,7,10,13,16,19-hexaoxa-26-thia-22-azanonacosan-29-oic Acid (C-14bSR).

### Example 15

### Synthesis of 1-((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)-1H-pyrrole-2,5-dione (C-15)

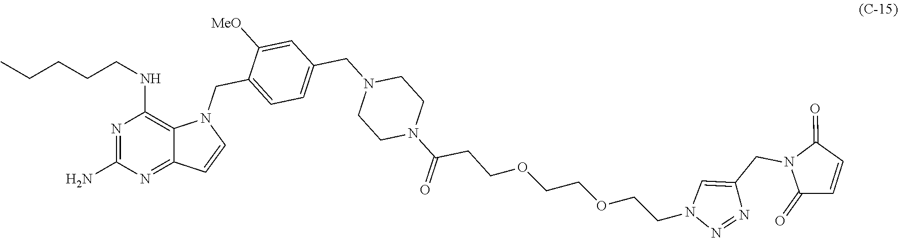 [SMILES: CCCCCNc1nc(N)nc2ccn(Cc3ccc(CN4CCN(C(=O)CCOCCOCCn5cc(CN6C(=O)C=CC6=O)nn5)CC4)cc3OC)c12]

Step 1: 1-(4-(4-((2-amino-4-(pentylamino)-5H-pyrrolo[3,2-d]pyrimidin-5-yl)methyl)-3-methoxybenzyl)piperazin-1-yl)-3-(2-(2-azidoethoxy)ethoxy)propan-1-one was prepared following the procedure similar to Example 1, except 3-(2-(2-azidoethoxy)ethoxy)propanoic acid was used in place of 3-(2,5-dioxo-2,5-dihydro-1H-pyrrol-1-yl)propanoic acid.

Step 2: A round bottom flask was charged with 1-(4-(4-((2-amino-4-(pentylamino)-5H-pyrrolo[3,2-d]pyrimidin-5-yl)methyl)-3-methoxybenzyl)piperazin-1-yl)-3-(2-(2-azidoethoxy)ethoxy)propan-1-one (1.0 equiv.), CuSO₄ (0.25 equiv.), L-Ascorbic acid sodium salt (1.1 equiv.), 1-(prop-2-yn-1-yl)-1H-pyrrole-2,5-dione (2.2 equiv.), and a mixture oft-BuOH/water (1:1, v/v, 0.012 M). The reaction mixture was placed under vacuum and subsequently flushed with N₂ (this was repeated four more times). The reaction mixture was then stirred at room temperature for 2 hours and the crude reaction mixture was then purified by RP-HPLC (0.035% TFA in ACN:0.05% TFA in H₂O, C18 column) to afford 1-((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)-1H-pyrrole-2,5-dione (C-15) as a solid as the TFA salt: ¹H NMR (CD₃OD): δ 7.94 (s, 1H), 7.37 (d, 1H), 7.29 (s, 1H), 7.05 (d, 1H), 6.85 (s, 2H), 6.81 (d, 1H), 6.24 (d, 1H), 5.57 (s, 2H), 4.73 (s, 2H), 4.52 (t, 2H), 4.36 (s, 2H), 3.95 (s, 3H), 3.85 (t, 2H), 3.84 (m, 4H), 3.66 (t, 2H), 3.54 (m, 6H), 3.27 (m, 4H), 2.63 (t, 2H), 1.53 (m, 2H), 1.30 (m, 2H), 1.19 (m, 2H), 0.88 (t, 3H). LCMS [M+H]=758.4.

### Example 16

### Synthesis of 3-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic Acid (C-16a) and 2-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic Acid (C-16b)

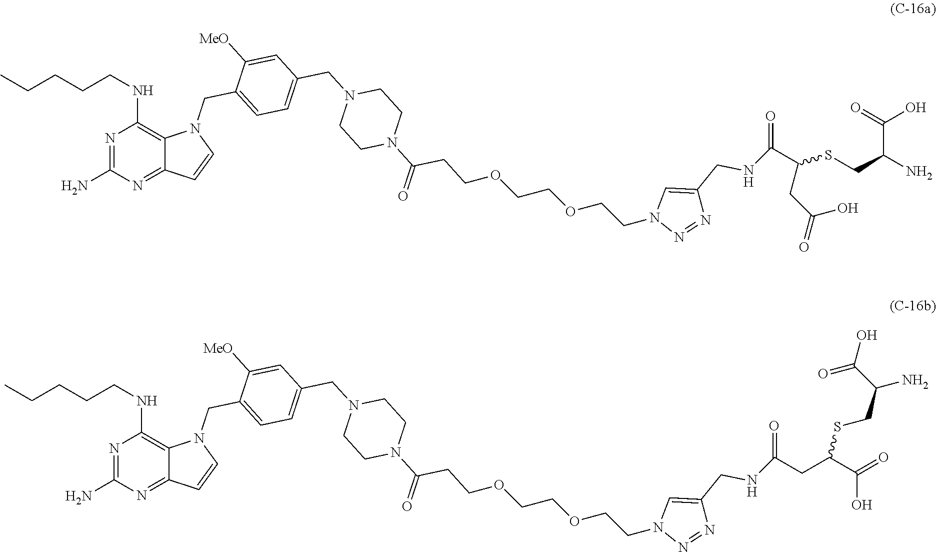 [SMILES: CCCCCNc1nc(N)nc2ccn(Cc3ccc(CN4CCN(C(=O)CCOCCOCCn5cc(CNC(=O)C(CC(=O)O)SC[C@H](N)C(=O)O)nn5)CC4)cc3OC)c12 ; CCCCCNc1nc(N)nc2ccn(Cc3ccc(CN4CCN(C(=O)CCOCCOCCn5cc(CNC(=O)CC(SC[C@H](N)C(=O)O)C(=O)O)nn5)CC4)cc3OC)c12]

3-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic acid (C-16a) and 2-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic acid (C-16b) were prepared following a procedure similar to Example 4, except Compound (C-15) was used in place of Compound (C-1), to afford a mixture of Compounds (C-16a) and (C-16b), as their respective diasteromers (Compounds (C-16aSR), C-16aRR), (C-16bRR) and (C-16bRR) below), as a solid as the TFA salt. The crude reaction mixture was purified by RP-HPLC (0.035% TFA in ACN:0.05% TFA in H₂O, C18 column): ¹H NMR (CD₃OD): δ 7.91 (s, 1H), 7.36 (d, 1H), 7.30 (s, 1H), 7.06 (d, 1H), 6.80 (d, 1H), 6.24 (d, 1H), 5.57 (s, 2H), 4.54 (s, 2H), 4.44 (m, 2H), 4.34 (s, 2H), 4.25 (m, 1H), 3.95 (s, 3H), 4.83 (m, 6H), 3.68 (t, 2H), 3.55 (m, 6H), 3.25 (m, 2H), 2.86 (m, 1H), 2.64 (m, 2H), 1.53 (m, 2H), 1.30 (m, 2H), 1.19 (m, 2H), 0.88 (t, 3H). LCMS [M+H]=897.4

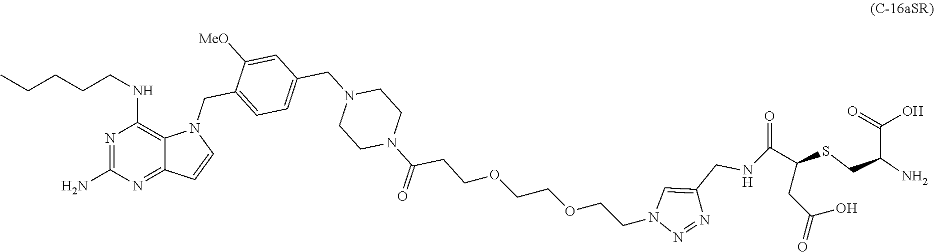 [SMILES: CCCCCNc1nc(N)nc2ccn(Cc3ccc(CN4CCN(C(=O)CCOCCOCCn5cc(CNC(=O)[C@H](CC(=O)O)SC[C@H](N)C(=O)O)nn5)CC4)cc3OC)c12]

(R)-3-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic Acid (C-16aSR);

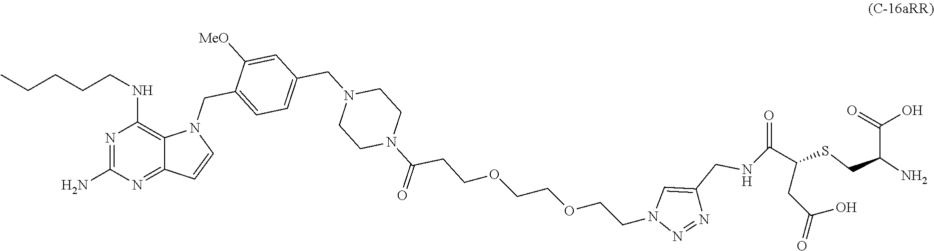 [SMILES: CCCCCNc1nc(N)nc2ccn(Cc3ccc(CN4CCN(C(=O)CCOCCOCCn5cc(CNC(=O)[C@@H](CC(=O)O)SC[C@H](N)C(=O)O)nn5)CC4)cc3OC)c12]

(R)-3-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic Acid (C-16aRR);

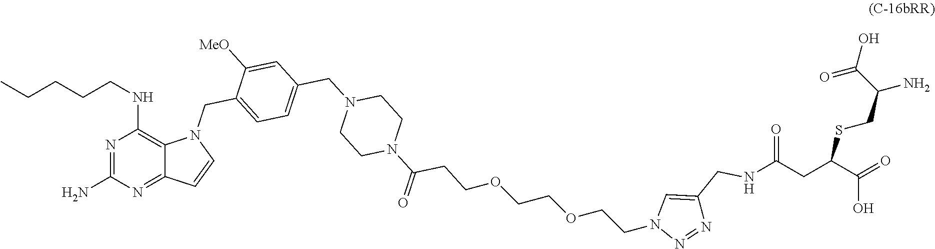 [SMILES: CCCCCNc1nc(N)nc2ccn(Cc3ccc(CN4CCN(C(=O)CCOCCOCCn5cc(CNC(=O)C[C@@H](SC[C@H](N)C(=O)O)C(=O)O)nn5)CC4)cc3OC)c12]

(R)-2-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic Acid (C-16bRR);

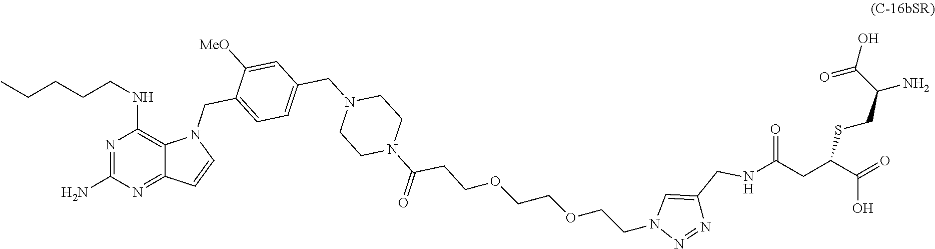 [SMILES: CCCCCNc1nc(N)nc2ccn(Cc3ccc(CN4CCN(C(=O)CCOCCOCCn5cc(CNC(=O)C[C@H](SC[C@H](N)C(=O)O)C(=O)O)nn5)CC4)cc3OC)c12]

(S)-2-(((R)-2-amino-2-carboxyethyl)thio)-4-(((1-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-1H-1,2,3-triazol-4-yl)methyl)amino)-4-oxobutanoic Acid (C-16bSR).

### Example 17

### Synthesis of N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-3-(2,5-dioxo-2,5-dihydro-1H-pyrrol-1-yl)propanamide (C-17)

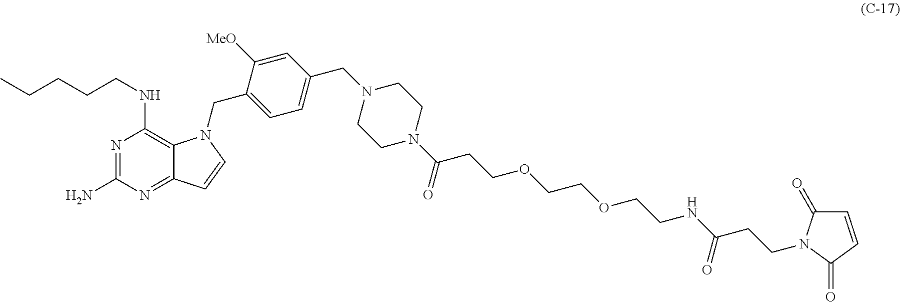 [SMILES: CCCCCNc1nc(N)nc2ccn(Cc3ccc(CN4CCN(C(=O)CCOCCOCCNC(=O)CCN5C(=O)C=CC5=O)CC4)cc3OC)c12]

N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-3-(2,5-dioxo-2,5-dihydro-1H-pyrrol-1-yl)propanamide (C-17) was prepared following a procedure similar to Example 1, except 3-(2-(2-(3-(2,5-dioxo-2,5-dihydro-1H-pyrrol-1-yl)propanamido)ethoxy)ethoxy)propanoic acid was used in place of 3-(2,5-dioxo-2,5-dihydro-1H-pyrrol-1-yl)propanoic acid, to afford N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-3-(2,5-dioxo-2,5-dihydro-1H-pyrrol-1-yl)propanamide (C-17) as a solid as the TFA salt: ¹H NMR (CD₃OD): δ 7.37 (d, 1H), 7.28 (d, 1H), 7.06 (d, 1H), 6.82 (d, 1H), 6.80 (s, 2H), 6.24 (d, 1H), 5.58 (s, 2H), 4.37 (s, 2H), 3.96 (s, 3H), 3.84 (m, 4H), 3.40 (m, 4H), 3.56 (m, 6H), 3.48 (t, 2H), 3.20 (m, 6H), 2.69 (t, 2H), 2.45 (t, 2H), 1.53 (m, 2H), 1.31 (m, 2H), 1.19 (m, 2H), 0.88 (t, 3H). LCMS [M+H]=748.4.

### Example 18

### Synthesis of (19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-(carboxymethyl)-1,11,15-trioxo-4,7-dioxa-17-thia-10,14-diazaicosan-20-oic Acid (C-18a) and (20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic Acid (C-18b)

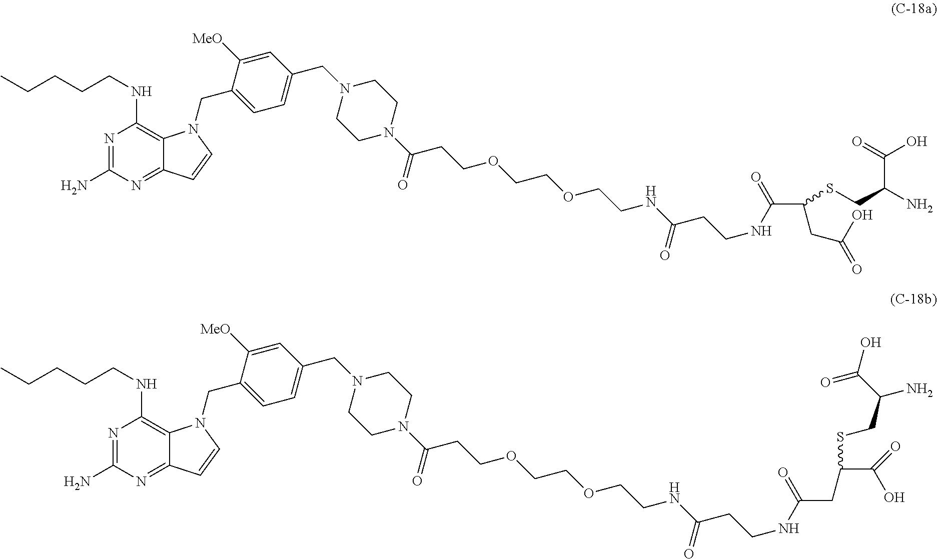 [SMILES: CCCCCNc1nc(N)nc2ccn(Cc3ccc(CN4CCN(C(=O)CCOCCOCCNC(=O)CCNC(=O)C(CC(=O)O)SC[C@H](N)C(=O)O)CC4)cc3OC)c12 ; CCCCCNc1nc(N)nc2ccn(Cc3ccc(CN4CCN(C(=O)CCOCCOCCNC(=O)CCNC(=O)CC(SC[C@H](N)C(=O)O)C(=O)O)CC4)cc3OC)c12]

(19R)-19-amino-1-(4-(4-((2-amino-4-(pentylamino)-5H-pyrrolo[3,2-d]pyrimidin-5-yl)methy)-3-methoxybenzyl)piperazin-1-yl)-16-(carboxymethyl)-1,11,15-trioxo-4,7-dioxa-17-thia-10,14-diazaicosan-20-oic acid (C-18a) and (20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic acid (C-18b) were prepared following a procedure similar to Example 4, except Compound (C-17) was used in place of Compound (C-1), to afford a mixture of Compounds (C-18a) and (C-18b), as their respective diasteromers (Compounds (C-18aSR), C-18aRR), (C-18bRR) and (C-18bRR) below), as a solid as the TFA salt. The crude reaction mixture was purified by RP-HPLC (0.035% TFA in ACN:0.05% TFA in H₂O, C18 column): ¹H NMR (CD₃OD): δ 7.37 (d, 1H), 7.30 (s, 1H), 7.07 (d, 1H), 6.80 (d, 1H), 6.25 (d, 1H), 5.57 (s, 2H), 4.35 (s, 2H), 4.19 (m, 1H), 3.95 (s, 3H), 3.89 (s, 3H), 3.76 (m, 3H), 3.60 (s, 4H), 3.53 (m, 4H), 3.41 (m, 1H), 3.36 (m, 2H), 3.22 (s, 2H), 2.70 (t, 2H), 2.42 (2H), 1.53 (m, 2H), 1.30 (m, 2H), 1.19 (m, 2H), 0.88 (t, 3H). LCMS [M+H]=887.4.

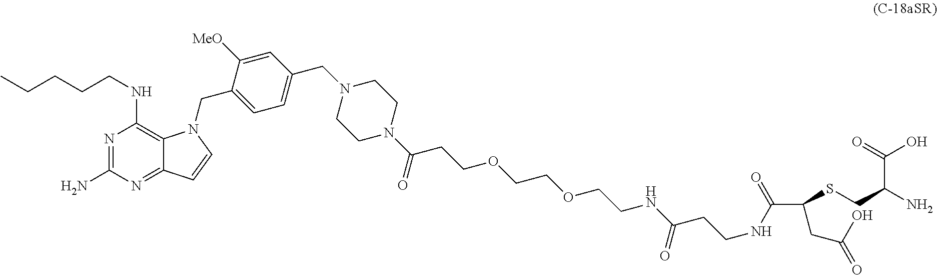 [SMILES: CCCCCNc1nc(N)nc2ccn(Cc3ccc(CN4CCN(C(=O)CCOCCOCCNC(=O)CCNC(=O)[C@H](CC(=O)O)SC[C@H](N)C(=O)O)CC4)cc3OC)c12]

(16S,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-(carboxymethyl)-1,11,15-trioxo-4,7-dioxa-17-thia-10,14-diazaicosan-20-oic Acid (C-18aSR);

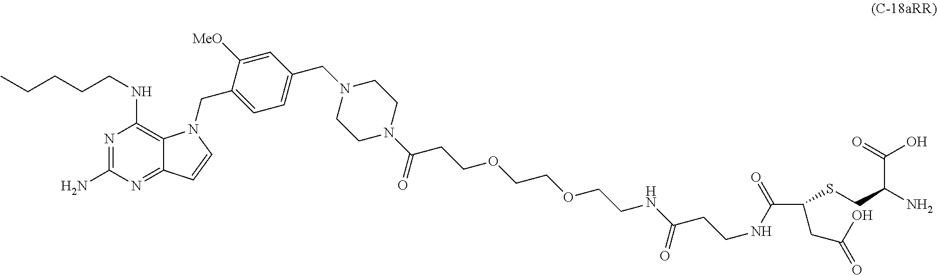 [SMILES: CCCCCNc1nc(N)nc2ccn(Cc3ccc(CN4CCN(C(=O)CCOCCOCCNC(=O)CCNC(=O)[C@@H](CC(=O)O)SC[C@H](N)C(=O)O)CC4)cc3OC)c12]

(16R,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-(carboxymethyl)-1,11,15-trioxo-4,7-dioxa-17-thia-10,14-diazaicosan-20-oic Acid (C-18aRR);

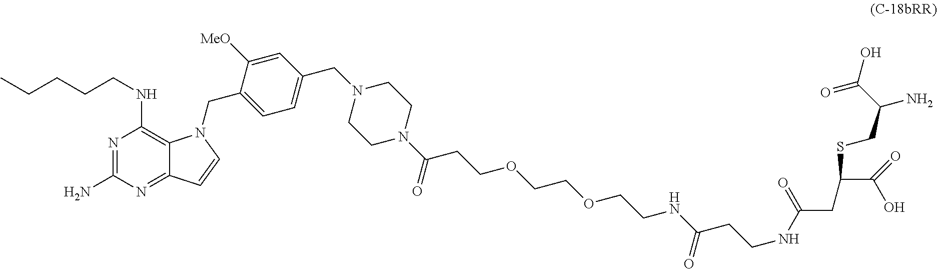 [SMILES: CCCCCNc1nc(N)nc2ccn(Cc3ccc(CN4CCN(C(=O)CCOCCOCCNC(=O)CCNC(=O)C[C@@H](SC[C@H](N)C(=O)O)C(=O)O)CC4)cc3OC)c12]

(17R,20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic Acid (C-18bRR);

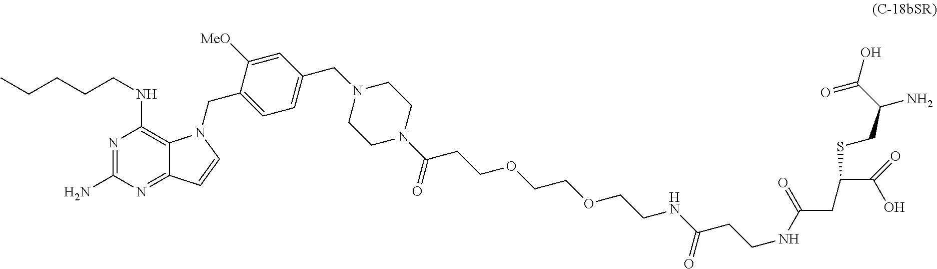 [SMILES: CCCCCNc1nc(N)nc2ccn(Cc3ccc(CN4CCN(C(=O)CCOCCOCCNC(=O)CCNC(=O)C[C@H](SC[C@H](N)C(=O)O)C(=O)O)CC4)cc3OC)c12]

(17S,20R)-20-amino-1-(4-(4-((2-amino-4-(pentylamino)-5H-pyrrolo[3,2-d]pyrimidin-5-yl)methyl)-3-methoxybenzyl)piperazin-1-yl)-17-carboxy-1,11,15-trioxo-4,7-dioxa-18-thia-10,14-diazahenicosan-21-oic Acid (C-18bSR).

### Example 19

### Synthesis of 5-(4-((4-(3-aminopropyl)piperazin-1-yl)methyl)-2-methoxybenzyl)-N4-pentyl-5H-pyrrolo[3,2-d]pyrimidine-2,4-diamine (C-19)

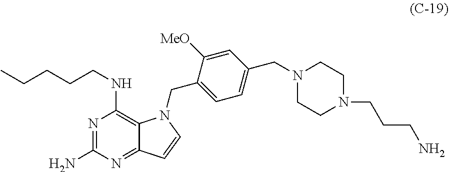 [SMILES: CCCCCNc1nc(N)nc2ccn(Cc3ccc(CN4CCN(CCCN)CC4)cc3OC)c12]

5-(4-((4-(3-aminopropyl)piperazin-1-yl)methyl)-2-methoxybenzyl)-N4-pentyl-5H-pyrrolo[3,2-d]pyrimidine-2,4-diamine (C-19) was prepared by a two step sequence. In the first step a round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), tert-butyl (3-bromopropyl)carbamate (1.2 equiv.), Huenig's base (2.4 equiv.), and DMF (0.2 M). The reaction mixture was heated to 60° C. and then stirred for 18 hours. The crude reaction mixture was then cooled to room temperature and purified by ISCO chromatography (0-20% MeOH:DCM) to provide the intermediate tert-butyl (3-(4-(4-((2-amino-4-(pentylamino)-5H-pyrrolo[3,2-d]pyrimidin-5-yl)methyl)-3-methoxybenzyl)piperazin-1-yl)propyl)carbamate. In the second step a procedure similar to the last step in the synthesis of (Int-1) was used to obtain 5-(4-((4-(3-aminopropyl)piperazin-1-yl)methyl)-2-methoxybenzyl)-N4-pentyl-5H-pyrrolo[3,2-d]pyrimidine-2,4-diamine (C-19) as a solid: 1H NMR (CD₃OD): δ 7.24 (d, 1H), 7.10 (d, 1H), 6.85 (d, 1H), 6.57 (d, 1H), 6.11 (s, 1H), 5.42 (s, 2H), 3.95 (s, 3H), 3.52 (s, 2H), 3.35 (m, 2H), 2.80 (t, 2H), 2.51 (m, 4H), 2.45 (m, 4H), 1.72 (m, 2H), 1.40 (m, 2H), 1.28 (m, 4H), 1.15 (m, 2H), 0.88 (t, 3H). LRMS [M+H]=495.3.

### Example 20

### Synthesis of 1-(4-(4-((2-amino-4-(pentylamino)-5H-pyrrolo[3,2-d]pyrimidin-5-yl)methyl)-3-methoxybenzyl)piperazin-1-yl)-3-(2-(2-aminoethoxy)ethoxy)propan-1-one (C-20)

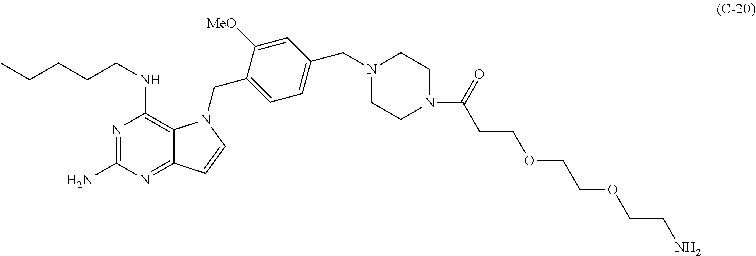 [SMILES: CCCCCNc1nc(N)nc2ccn(Cc3ccc(CN4CCN(C(=O)CCOCCOCCN)CC4)cc3OC)c12]

1-(4-(4-((2-amino-4-(pentylamino)-5H-pyrrolo[3,2-d]pyrimidin-5-yl)methyl)-3-methoxybenzyl)piperazin-1-yl)-3-(2-(2-aminoethoxy)ethoxy)propan-1-one (C-20) was prepared following a procedure of Example 19, except 2,2-dimethyl-4-oxo-3,8,11-trioxa-5-azatetradecan-14-oic acid was used in place of tert-butyl (3-bromopropyl)carbamate, to afford 1-(4-(4-((2-amino-4-(pentylamino)-5H-pyrrolo[3,2-d]pyrimidin-5-yl)methyl)-3-methoxybenzyl)piperazin-1-yl)-3-(2-(2-aminoethoxy)ethoxy)propan-1-one (C-20) as a solid: ¹H NMR (CD₃OD): δ 7.24 (d, 1H), 7.11 (s, 1H), 6.86 (d, 1H), 6.57 (d, 1H), 6.12 (d, 1H), 5.42 (s, 2H), 3.96 (s, 3H), 3.76 (t, 2H), 3.59 (m, 12H), 3.37 (t, 2H), 2.76 (t, 2H), 2.66 (t, 2H), 2.45 (m, 4H), 1.41 (m, 2H), 1.28 (m, 2H), 1.16 (m, 2H), 0.89 (t, 3H). LRMS [M+H]=597.4.

### Example 21

### Synthesis of N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(2,5-dioxo-2,5-dihydro-1H-pyrrol-1-yl)acetamide (C-21)

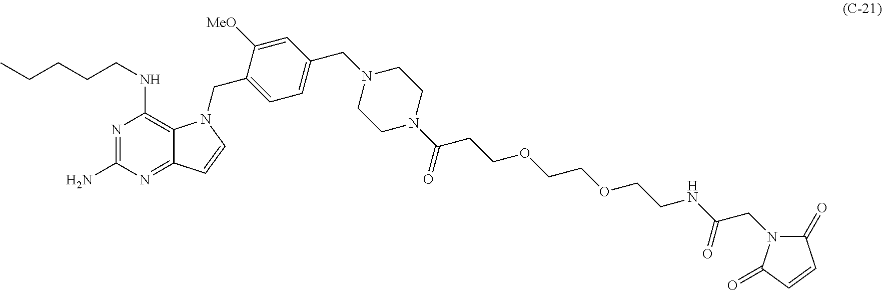 [SMILES: CCCCCNc1nc(N)nc2ccn(Cc3ccc(CN4CCN(C(=O)CCOCCOCCNC(=O)CN5C(=O)C=CC5=O)CC4)cc3OC)c12]

A round bottom flask was charged with 1-(4-(4-((2-amino-4-(pentylamino)-5H-pyrrolo[3,2-d]pyrimidin-5-yl)methyl)-3-methoxybenzyl)piperazin-1-yl)-3-(2-(2-aminoethoxy)ethoxy)propan-1-one (C-20) (1.0 equiv.), DIEA (10.0 equiv.) and DMF (0.004 M) and the mixture was stirred at room temperature for 15 minutes. A separate flask was then charged with 2,5-dioxopyrrolidin-1-yl 2-(2,5-dioxo-2,5-dihydro-1H-pyrrol-1-yl)acetate (1.5 equiv.), DIEA (10.0 equiv.) and DMF (0.006 M). This mixture was also stirred for 15 minutes at room temperature and then the two solutions were mixed and the reaction mixture stirred at room temperature for 1 hour. The crude reaction mixture was purified by RP-HPLC (0.035% TFA in ACN:0.05% TFA in H₂O, C18 column) to afford N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(2,5-dioxo-2,5-dihydro-1H-pyrrol-1-yl)acetamide (C-21) as a solid as the TFA salt: ¹H NMR (CD₃CN): δ 7.30 (d, 1H), 7.05 (s, 1H), 6.98 (s, 1H), 6.86 (d, 1H), 6.82 (s, 2H), 6.74 (s, 1H), 6.68 (d, 1H), 6.21 (d, 1H), 6.08 (t, 1H), 5.38 (s, 2H), 4.08 (s, 2H), 3.89 (s, 3H), 3.70 (t, 2H), 3.41 (m, 14H), 3.29 (m, 2H), 2.55, (t, 2H), 2.38 (m, 4H), 1.41 (m, 2H), 1.26 (m, 2H), 1.13 (m, 2H), 0.85 (t, 3H). LCMS [M+H]=734.4.

### Example 22

### Synthesis of (2R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic Acid (C-22a) and (19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic Acid (C-22b)

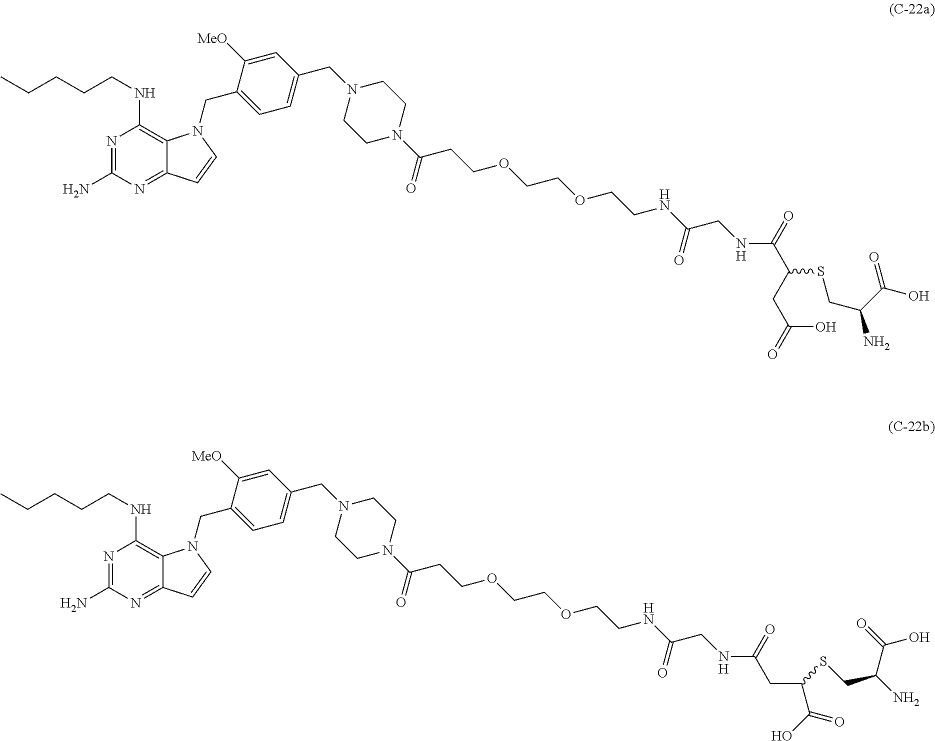 [SMILES: CCCCCNc1nc(N)nc2ccn(Cc3ccc(CN4CCN(C(=O)CCOCCOCCNC(=O)CNC(=O)C(CC(=O)O)SC[C@H](N)C(=O)O)CC4)cc3OC)c12 ; CCCCCNc1nc(N)nc2ccn(Cc3ccc(CN4CCN(C(=O)CCOCCOCCNC(=O)CNC(=O)CC(SC[C@H](N)C(=O)O)C(=O)O)CC4)cc3OC)c12]

(2R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic acid (C-22a) and (19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic acid (C-22b) were prepared following a procedure similar to Example 4, except Compound (C-21) was used in place of Compound (C-1), to afford a mixture of Compounds (C-22a) and (C-22b), as their respective diasteromers (Compounds (C-22aSR), C-22aRR), (C-22bRR) and (C-22bRR) below), as a solid as the TFA salt. The crude reaction mixture was purified by RP-HPLC (0.035% TFA in ACN:0.05% TFA in H₂O, C18 column): ¹H NMR (CD₃OD): δ 7.37 (d, 1H), 7.32 (s, 1H), 7.08 (d, 1H), 6.81 (d, 1H), 6.24 (d, 1H), 5.57 (s, 2H), 4.34 (s, 2H), 4.20 (m, 1H), 3.96 (s, 3H), 3.82 (m, 9H), 3.56 (m, 9H), 3.38 (m, 3H), 3.21 (m, 2H), 2.70 (t, 2H), 1.54 (m, 2H), 1.32 (m, 2H), 1.19 (m, 2H), 0.89 (t, 3H). LCMS [M+H]=873.4.

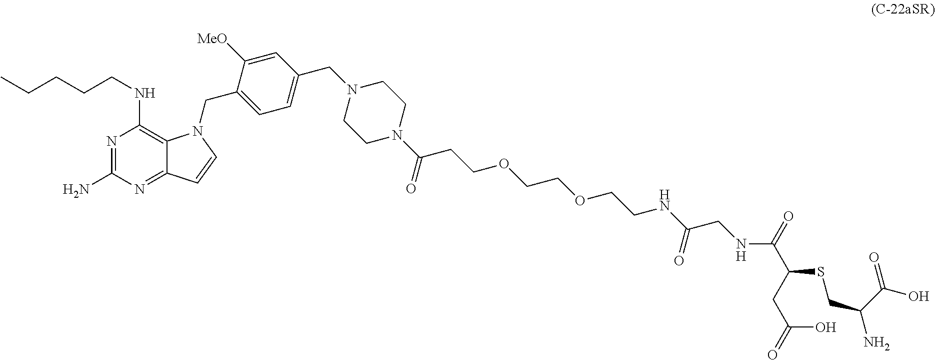 [SMILES: CCCCCNc1nc(N)nc2ccn(Cc3ccc(CN4CCN(C(=O)CCOCCOCCNC(=O)CNC(=O)[C@H](CC(=O)O)SC[C@H](N)C(=O)O)CC4)cc3OC)c12]

(2R,5S)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic Acid (C-22aSR);

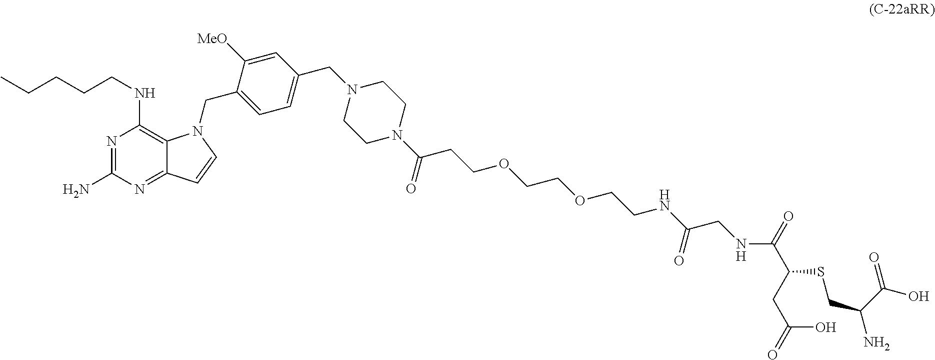 [SMILES: CCCCCNc1nc(N)nc2ccn(Cc3ccc(CN4CCN(C(=O)CCOCCOCCNC(=O)CNC(=O)[C@@H](CC(=O)O)SC[C@H](N)C(=O)O)CC4)cc3OC)c12]

(2R,5R)-2-amino-19-(4-(4-((2-amino-4-(pentylamino)-5H-pyrrolo[3,2-d]pyrimidin-5-yl)methyl)-3-methoxybenzyl)piperazin-1-yl)-5-(carboxymethyl)-6,9,19-trioxo-13,16-dioxa-4-thia-7,10-diazanonadecan-1-oic Acid (C-22aRR);

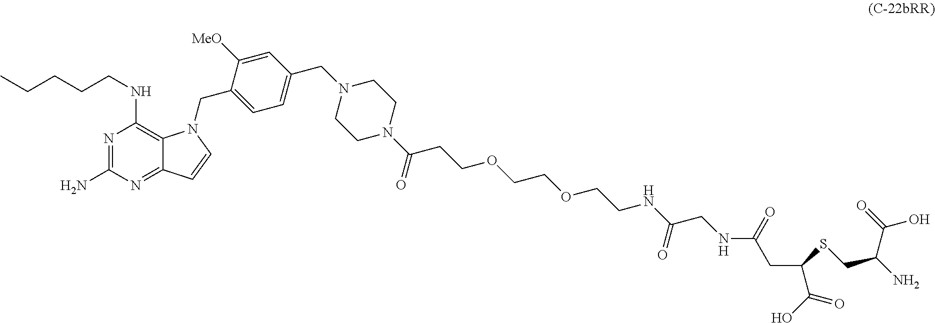 [SMILES: CCCCCNc1nc(N)nc2ccn(Cc3ccc(CN4CCN(C(=O)CCOCCOCCNC(=O)CNC(=O)C[C@@H](SC[C@H](N)C(=O)O)C(=O)O)CC4)cc3OC)c12]

(16R,19R)-19-amino-1-(4-(4-((2-amino-194-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic Acid (C-22bRR);

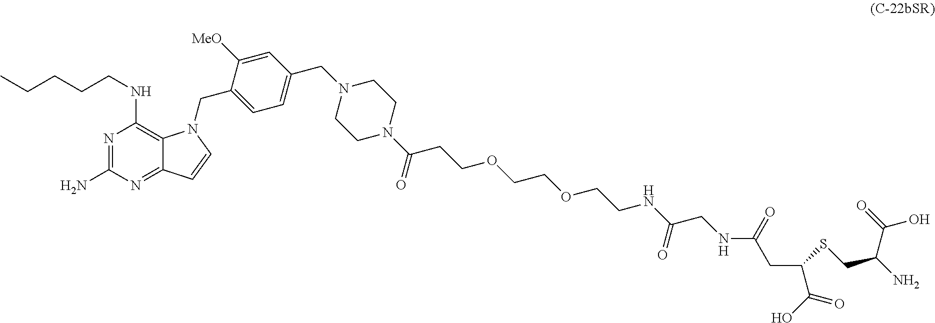 [SMILES: CCCCCNc1nc(N)nc2ccn(Cc3ccc(CN4CCN(C(=O)CCOCCOCCNC(=O)CNC(=O)C[C@H](SC[C@H](N)C(=O)O)C(=O)O)CC4)cc3OC)c12]

(16S,19R)-19-amino-1-(4-(4-((2-amino-4-(pentylamino)-5H-pyrrolo[3,2-d]pyrimidin-5-yl)methyl)-3-methoxybenzyl)piperazin-1-yl)-16-carboxy-1,11,14-trioxo-4,7-dioxa-17-thia-10,13-diazaicosan-20-oic Acid (C-22bSR).

### Example 23

### Synthesis of 4-(4-((2-amino-4-(pentylamino)-5H-pyrrolo[3,2-d]pyrimidin-5-yl)methyl)-3-methoxybenzyl)-N-(2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl)piperazine-1-carboxamide (C-23)

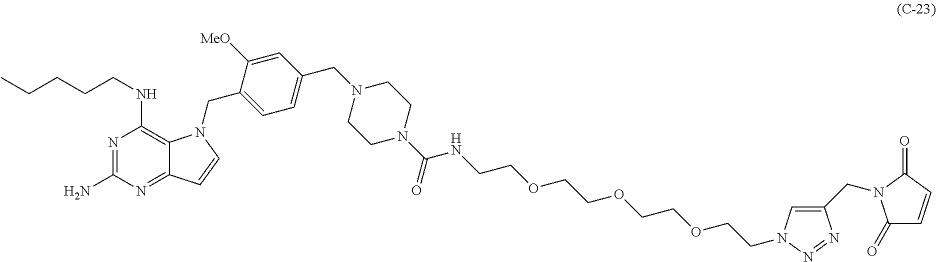 [SMILES: CCCCCNc1nc(N)nc2ccn(Cc3ccc(CN4CCN(C(=O)NCCOCCOCCOCCn5cc(CN6C(=O)C=CC6=O)nn5)CC4)cc3OC)c12]

A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1 equiv.), 4-nitrophenyl (2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl)carbamate (0.9 equiv.), triethylamine (3.0 equiv.) and DMSO (0.01 M). The reaction mixture was stirred at room temperature for 2 hours and the crude reaction mixture was then purified by RP-HPLC (0.035% TFA in ACN:0.05% TFA in H₂O, C18 column) to afford 4-(4-((2-amino-4-(pentylamino)-5H-pyrrolo[3,2-d]pyrimidin-5-yl)methyl)-3-methoxybenzyl)-N-(2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl)piperazine-1-carboxamide (C-23) as a solid as the TFA salt: ¹H NMR (CD₃OD): δ 7.96 (s, 1H), 7.36 (d, 1H), 7.26 (d, 1H), 7.05 (d, 1H), 6.85 (s, 2H), 6.79 (d, 1H), 6.24 (d, 1H), 5.57 (s, 2H), 4.74 (s, 2H), 4.53 (t, 2H), 4.35 (s, 2H), 3.95 (s, 3H), 3.86 (t, 2H), 3.85 (m, 4H), 3.54 (m, 12H), 3.22 (m, 6H), 1.53 (m, 2H), 1.30 (m, 2H), 1.19 (m, 2H), 0.88 (t, 3H). LCMS [M+H]=817.4.

Note: 4-nitrophenyl (2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl)carbamate

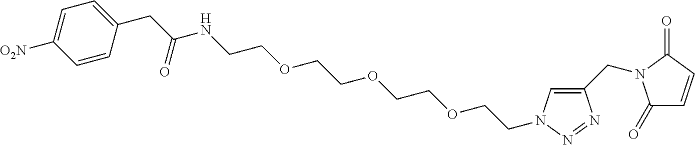 [SMILES: O=C(Cc1ccc([N+](=O)[O-])cc1)NCCOCCOCCOCCn1cc(CN2C(=O)C=CC2=O)nn1]

was prepared using the following procedure:

Step 1: Triethylamine (2.5 equiv.) and di-tert-butyl dicarbonate (1.1 equiv.) were added to a solution of 2-(2-(2-(2-azidoethoxy)ethoxy)ethoxy)ethanamine (1.0 equiv.) in CH₂Cl₂ (0.05 M) and the reaction mixture was stirred at room temperature for 30 minutes. The reaction mixture was then concentrated in vacuo and the residue was purified using RP-C18 ISCO and then lyophilized to give tert-butyl (2-(2-(2-(2-azidoethoxy)ethoxy)ethoxy)ethyl)carbamate.

Step 2: A solution of tert-butyl (2-(2-(2-(2-azidoethoxy)ethoxy)ethoxy)ethyl)carbamate (1 equiv.) and 1-(prop-2-yn-1-yl)-1H-pyrrole-2,5-dione (2.0 equiv.) in t-BuOH (0.08 M) was flushed with N₂ gas five times and then L-ascorbic acid sodium salt (1.0 equiv. 0.16 M in H₂O) and CuSO₄ (0.2 equiv. 0.03 M in H₂O) were added. The reaction mixture was again flushed with N₂ gas five times and then stirred at room temperature for 4 h. The reaction mixture was then purified by ISCO RP-C18 and lyophilized to afford tert-butyl (2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl) carbamate.

Step 3: A solution of tert-butyl (2-(2-(2-(2-(4-((2,5-dioxo-2,5-dihydro-1H-pyrrol-1-yl)methyl)-1H-1,2,3-triazol-1-yl)ethoxy)ethoxy)ethoxy)ethyl) carbamate in TFA (0.02 M) was concentrated in vacuo to afford 1-((1-(23-amino-3,6,9,12,15,18,21-heptaoxatricosyl)-1H-1,2,3-triazol-4-yl)methyl)-1H-pyrrole-2,5-dione. LCMS [M+H]=354.2.

Step 4: 4-Nitrophenyl carbonochloridate (1.10 equiv.) and triethylamine (2.50 equiv.) were added to a solution of 1-((1-(2-(2-(2-(2-aminoethoxy)ethoxy)ethoxy)ethyl)-1H-1,2,3-triazol-4-yl)methyl)-1H-pyrrole-2,5-dione (1 equiv.) in CH₂Cl₂ (0.01 M) and the reaction mixture was stirred at room temperature for 10 minutes. The reaction mixture was then concentrated in vacuo, purified by RP-C18 ISCO and then lyophilized to afford 4-nitrophenyl (2-(2-(2-(2-(4-((2,5-dioxo-2,5-dihydro-1H-pyrrol-1-yl)methyl)-1H-1,2,3-triazol-1-yl)ethoxy)ethoxy)ethoxy) ethyl)carbamate LCMS [M+H]=519.2.

### Example 24

### Synthesis of 3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic Acid (C-24a) and 2-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic Acid (C-24b)

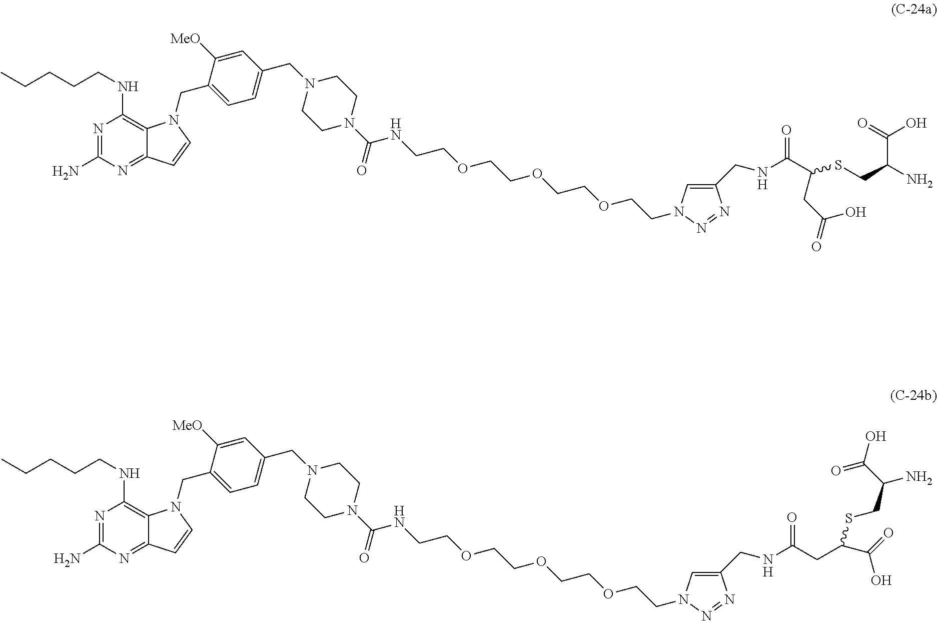 [SMILES: CCCCCNc1nc(N)nc2ccn(Cc3ccc(CN4CCN(C(=O)NCCOCCOCCOCCn5cc(CNC(=O)C(CC(=O)O)SC[C@H](N)C(=O)O)nn5)CC4)cc3OC)c12 ; CCCCCNc1nc(N)nc2ccn(Cc3ccc(CN4CCN(C(=O)NCCOCCOCCOCCn5cc(CNC(=O)CC(SC[C@H](N)C(=O)O)C(=O)O)nn5)CC4)cc3OC)c12]

3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic acid (C-24a) and 2-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl) piperazin-1-yl)-1-oxo-5,8,11-trioxa-2-azatridecan-13-yl)-1H-1,2,3-triazol-4-yl)methyl)amino)-4-oxobutanoic acid (C-24b) were prepared following a procedure similar to Example 4, except Compound (C-23) was used in place of Compound (C-1), to provide a mixture of Compounds (C-24a) and (C-24b), as their respective diasteromers (Compounds (C-24aSR), C-24aRR), (C-24bRR) and (C-24bRR) below), as a solid as the TFA salt. The crude reaction mixture was purified by RP-HPLC (0.035% TFA in ACN:0.05% TFA in H₂O, C18 column): LCMS [M+H]=956.4.

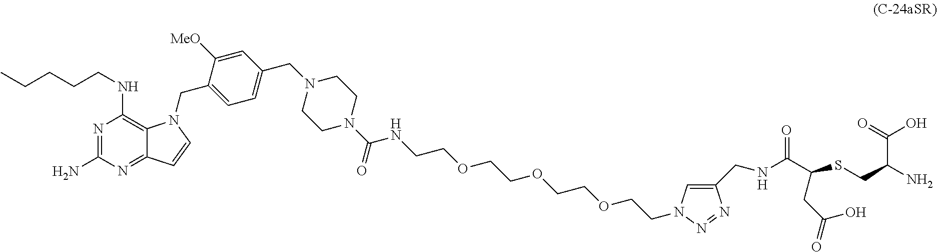 [SMILES: CCCCCNc1nc(N)nc2ccn(Cc3ccc(CN4CCN(C(=O)NCCOCCOCCOCCn5cc(CNC(=O)[C@H](CC(=O)O)SC[C@H](N)C(=O)O)nn5)CC4)cc3OC)c12]

(S)-3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic Acid (C-24aSR);

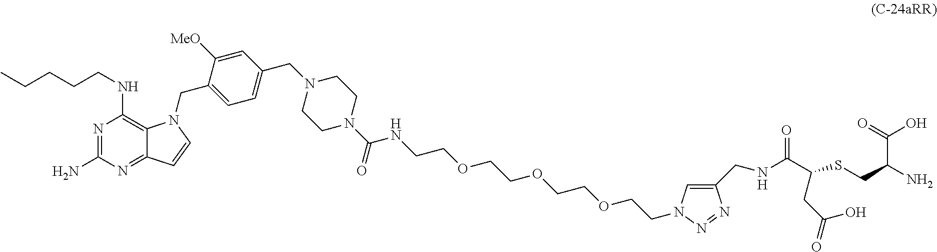 [SMILES: CCCCCNc1nc(N)nc2ccn(Cc3ccc(CN4CCN(C(=O)NCCOCCOCCOCCn5cc(CNC(=O)[C@@H](CC(=O)O)SC[C@H](N)C(=O)O)nn5)CC4)cc3OC)c12]

(R)-3-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic Acid (C-24aRR);

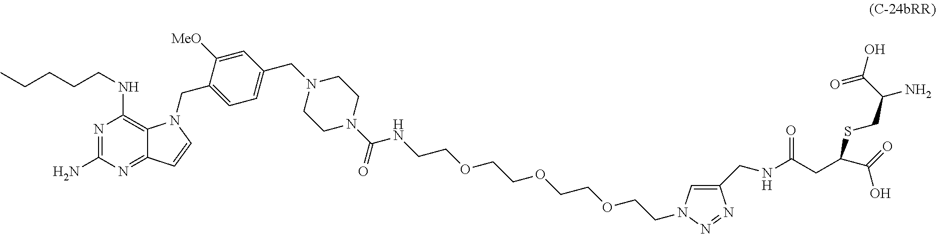 [SMILES: CCCCCNc1nc(N)nc2ccn(Cc3ccc(CN4CCN(C(=O)NCCOCCOCCOCCn5cc(CNC(=O)C[C@@H](SC[C@H](N)C(=O)O)C(=O)O)nn5)CC4)cc3OC)c12]

(R)-2-(((R)-2-amino-2-(carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic Acid (C-24bRR);

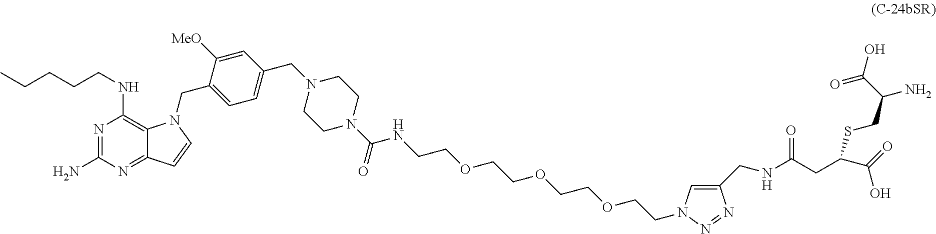 [SMILES: CCCCCNc1nc(N)nc2ccn(Cc3ccc(CN4CCN(C(=O)NCCOCCOCCOCCn5cc(CNC(=O)C[C@H](SC[C@H](N)C(=O)O)C(=O)O)nn5)CC4)cc3OC)c12]

(S)-2-(((R)-2-amino-2-carboxyethyl)thio)-4-(((1-(1-(4-(4-((2-amino-4-(pentylamino)-5H-pyrrolo[3,2-d]pyrimidin-5-yl)methyl)-3-methoxybenzyl)piperazin-1-yl)-1-oxo-5,8,11-trioxa-2-azatridecan-13-yl)-1H-1,2,3-triazol-4-yl)methyl)amino)-4-oxobutanoic Acid (C-24bSR).

### Example 25

### Synthesis of 1-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)-1H-pyrrole-2,5-dione (C-25)

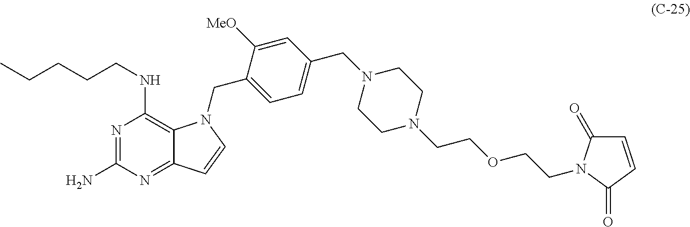 [SMILES: CCCCCNc1nc(N)nc2ccn(Cc3ccc(CN4CCN(CCOCCN5C(=O)C=CC5=O)CC4)cc3OC)c12]

A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), 2-(2-(2,5-dioxo-2,5-dihydro-1H-pyrrol-1-yl)ethoxy)acetaldehyde (4.0 equiv.), sodium cyanoborohydride (13.0 equiv.), and MeOH (0.04 M). The reaction mixture was stirred at room temperature for 1 hour. The crude reaction mixture was then purified by RP-HPLC (0.035% TFA in ACN:0.05% TFA in H₂O, C18 column) to afford 1-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)-1H-pyrrole-2,5-dione (C-25) as a solid as the TFA salt: ¹H NMR (CD₃OD): δ 7.36 (d, 1H), 7.16 (d, 1H), 6.96 (d, 1H), 6.83 (s, 2H), 6.76 (d, 1H), 6.23 (d, 1H), 5.53 (s, 2H), 3.93 (s, 3H), 3.84 (s, 2H), 3.78 (m, 2H), 3.71 (m, 2H), 3.64 (m, 2H), 3.54 (m, 2H), 3.35 (m, 4H), 3.27 (t, 2H), 2.95 (m, 4H), 1.52 (m, 2H), 1.30 (m, 2H), 1.19 (m, 2H), 0.88 (t, 3H). LCMS [M+H]=605.4.

Note: 2-(2-(2,5-dioxo-2,5-dihydro-1H-pyrrol-1-yl)ethoxy)acetaldehyde was prepared by adding 1-(2-(2-hydroxyethoxy)ethyl)-1H-pyrrole-2,5-dione (1.0 equiv.), Dess-Martin periodinane (1.5 equiv.) and DCM (0.1 M) to a round bottom flask and stirring the reaction mixture at room temperature for 2 hours. The reaction mixture was then filtered, the volatiles removed in vacuo and the product used without further purification.

### Example 26

### Synthesis of 3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic Acid (C-26a) and 2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic Acid (C-26b)

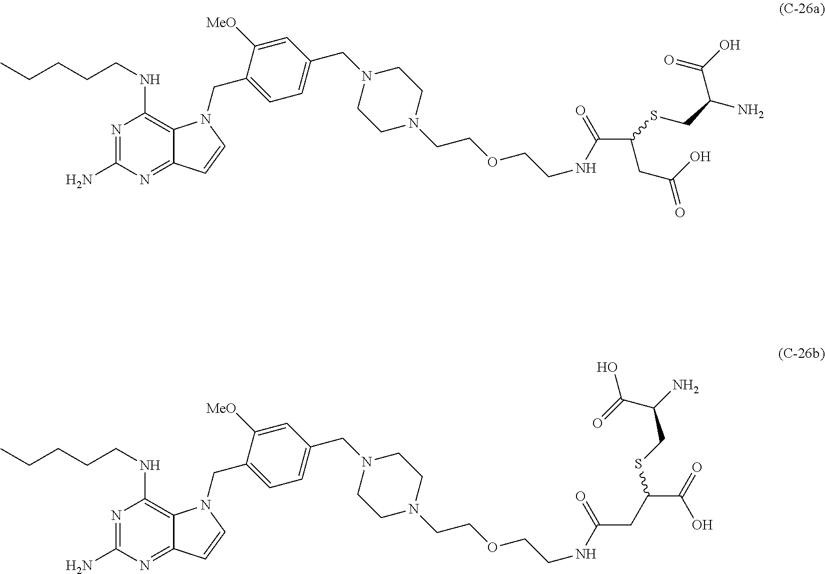 [SMILES: CCCCCNc1nc(N)nc2ccn(Cc3ccc(CN4CCN(CCOCCNC(=O)C(CC(=O)O)SC[C@H](N)C(=O)O)CC4)cc3OC)c12 ; CCCCCNc1nc(N)nc2ccn(Cc3ccc(CN4CCN(CCOCCNC(=O)CC(SC[C@H](N)C(=O)O)C(=O)O)CC4)cc3OC)c12]

3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid (C-26) and 2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic acid (C-26b) were prepared following a procedure similar to Example 4, except Compound (C-25) was used in place of Compound (C-1), to afford a mixture of Compounds (C-26a) and (C-26b), as their respective diasteromers (Compounds (C-26aSR), C-26aRR), (C-26bRR) and (C-26bRR) below), as a solid as the TFA salt. The crude reaction mixture was purified by RP-HPLC (0.035% TFA in ACN:0.05% TFA in H₂O, C18 column): LCMS [M+H]=744.4

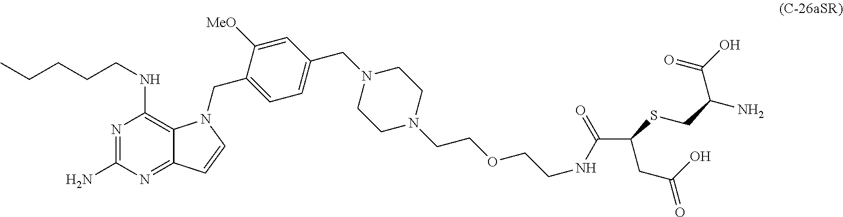 [SMILES: CCCCCNc1nc(N)nc2ccn(Cc3ccc(CN4CCN(CCOCCNC(=O)[C@H](CC(=O)O)SC[C@H](N)C(=O)O)CC4)cc3OC)c12]

(S)-3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic Acid (C-26aSR);

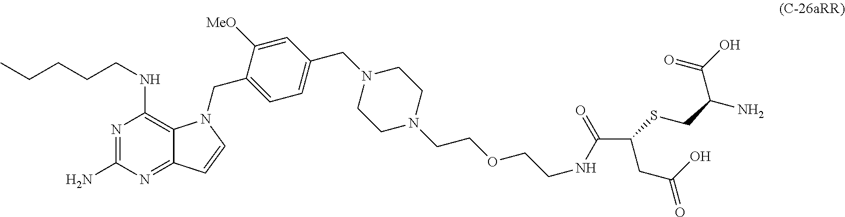 [SMILES: CCCCCNc1nc(N)nc2ccn(Cc3ccc(CN4CCN(CCOCCNC(=O)[C@@H](CC(=O)O)SC[C@H](N)C(=O)O)CC4)cc3OC)c12]

(R)-3-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic Acid (C-26aRR);

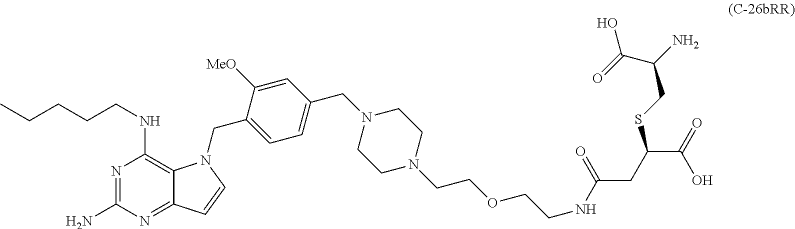 [SMILES: CCCCCNc1nc(N)nc2ccn(Cc3ccc(CN4CCN(CCOCCNC(=O)C[C@@H](SC[C@H](N)C(=O)O)C(=O)O)CC4)cc3OC)c12]

(R)-2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic Acid (C-26bRR);

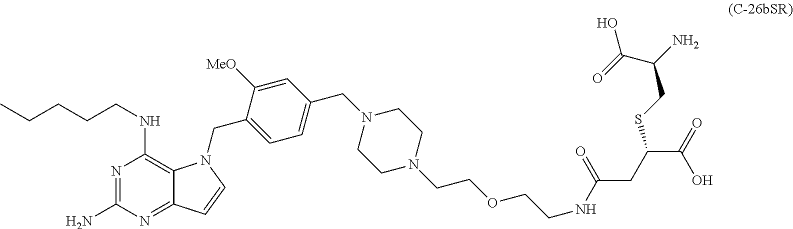 [SMILES: CCCCCNc1nc(N)nc2ccn(Cc3ccc(CN4CCN(CCOCCNC(=O)C[C@H](SC[C@H](N)C(=O)O)C(=O)O)CC4)cc3OC)c12]

(S)-2-(((R)-2-amino-2-carboxyethyl)thio)-4-((2-(2-(4-(4-((2-amino-4-(pentylamino)-5H-pyrrolo[3,2-d]pyrimidin-5-yl)methyl)-3-methoxybenzyl)piperazin-1-yl)ethoxy)ethyl)amino)-4-oxobutanoic Acid (C-26bSR).

### Example 27

### Synthesis of 1-((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)-1H-pyrrole-2,5-dione (C-27)

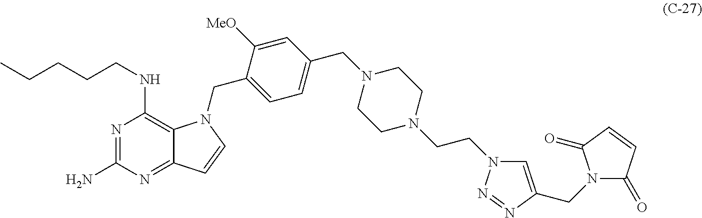 [SMILES: CCCCCNc1nc(N)nc2ccn(Cc3ccc(CN4CCN(CCn5cc(CN6C(=O)C=CC6=O)nn5)CC4)cc3OC)c12]

Step 1: A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), 2-azidoacetaldehyde (4.0 equiv.), sodium cyanoborohydride (32.0 equiv.), and MeOH (0.02 M). The reaction mixture was stirred at room temperature for 2 hours. The crude reaction mixture was then purified by RP-HPLC (0.035% TFA in ACN:0.05% TFA in H₂O, C18 column) to afford 5-(4-((4-(2-azidoethyl)piperazin-1-yl)methyl)-2-methoxybenzyl)-N4-pentyl-5H-pyrrolo[3,2-d]pyrimidine-2,4-diamine as a solid: LCMS [M+H]=507.3.

Step 2: A round bottom flask was charged with 5-(4-((4-(2-azidoethyl)piperazin-1-yl)methyl)-2-methoxybenzyl)-N4-pentyl-5H-pyrrolo[3,2-d]pyrimidine-2,4-diamine (1.0 equiv), 1-(prop-2-yn-1-yl)-1H-pyrrole-2,5-dione (2.3 equiv.) and a mixture of t-BuOH and water (2:1, v/v, 0.008 M). The reaction mixture was degassed under vacuum and flushed with N₂ five times to remove O₂. L-ascorbic acid sodium salt (1.1 equiv in 0.5 ml H₂O, degassed under and flushed with N₂five times to remove O₂) wad added using a syringe to the reaction mixture, then and CuSO₄ (0.2 equiv. in 0.5 ml water, degassed under vacuum and flushed with N₂ five times to remove O₂) was added using a syringe. The reaction mixture was then stirred at room temperature for 2 hours. The crude reaction mixture was then purified by RP-HPLC (0.035% TFA in ACN:0.05% TFA in H₂O, C18 column) to afford 1-((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)-1H-pyrrole-2,5-dione (C-27) as a solid as the TFA salt: ¹H NMR (CD₃OD): δ 7.95 (s, 1H), 7.36 (d, 1H), 7.22 (d, 1H), 7.02 (d, 1H), 6.86 (s, 2H), 6.79 (d, 1H), 6.23 (d, 1H), 5.57 (s, 2H), 4.76 (s, 2H), 4.52 (t, 2H), 4.26 (s, 2H), 3.95 (s, 3H), 3.54 (t, 2H), 2.85 (m, 8H), 2.94 (t, 2H), 1.53 (m, 2H), 1.31 (m, 2H), 1.18 (m, 2H), 0.88 (t, 3H). LCMS [M+H]=642.4.

Note: 2-azidoacetaldehyde was prepared by adding 2-azidoethanol (1.0 equiv.), Dess-Martin periodinane (1.5 equiv.) and DCM (0.20 M) to a round bottom flask and then stirring the reaction mixture at room temperature for 2 hours. The reaction mixture was then filtered, the volatiles removed in vacuo and the product used without further purification.

### Example 28

### Synthesis of 3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28a) and 2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28b)

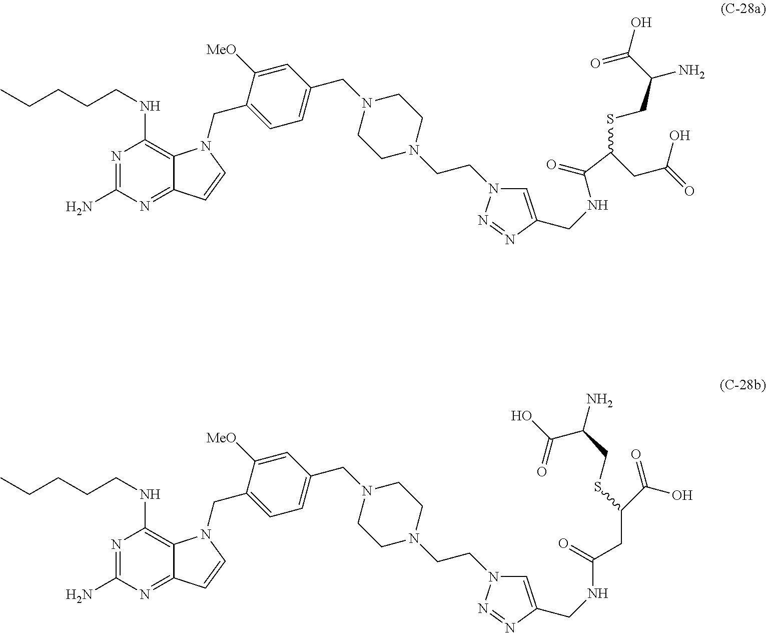 [SMILES: CCCCCNc1nc(N)nc2ccn(Cc3ccc(CN4CCN(CCn5cc(CNC(=O)C(CC(=O)O)SC[C@H](N)C(=O)O)nn5)CC4)cc3OC)c12 ; CCCCCNc1nc(N)nc2ccn(Cc3ccc(CN4CCN(CCn5cc(CNC(=O)CC(SC[C@H](N)C(=O)O)C(=O)O)nn5)CC4)cc3OC)c12]

3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28a) and 2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28b) were prepared following a procedure similar to Example 4, except Compound (C-27) was used in place of Compound (C-1), to afford a mixture of Compounds (C-28a) and (C-28b), as their respective diasteromers (Compounds (C-28aSR), C-28aRR), (C-28bRR) and (C-28bRR) below), as a solid as the TFA salt. The crude reaction mixture was purified by RP-HPLC (0.035% TFA in ACN:0.05% TFA in H₂O, C18 column): LCMS [M+H]=781.4

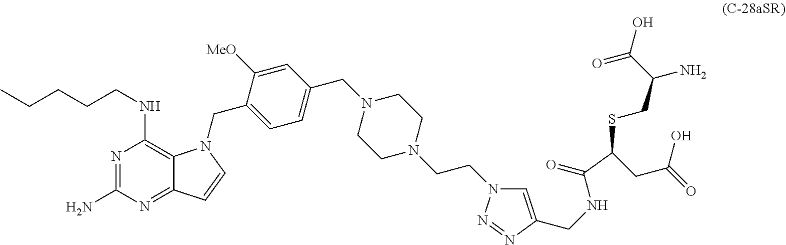 [SMILES: CCCCCNc1nc(N)nc2ccn(Cc3ccc(CN4CCN(CCn5cc(CNC(=O)[C@H](CC(=O)O)SC[C@H](N)C(=O)O)nn5)CC4)cc3OC)c12]

(S)-3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28aSR);

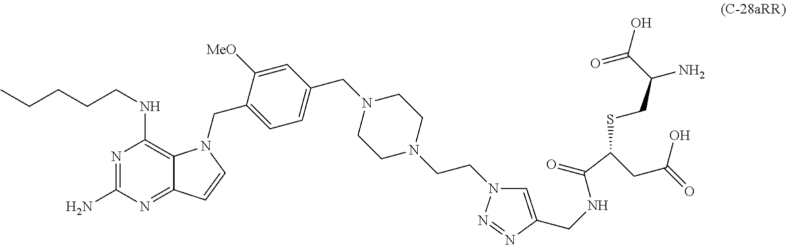 [SMILES: CCCCCNc1nc(N)nc2ccn(Cc3ccc(CN4CCN(CCn5cc(CNC(=O)[C@@H](CC(=O)O)SC[C@H](N)C(=O)O)nn5)CC4)cc3OC)c12]

(R)-3-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28aRR);

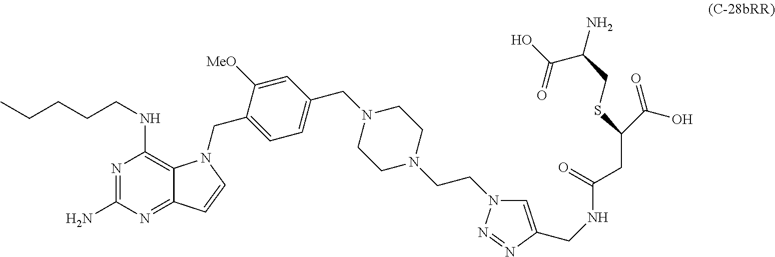 [SMILES: CCCCCNc1nc(N)nc2ccn(Cc3ccc(CN4CCN(CCn5cc(CNC(=O)C[C@@H](SC[C@H](N)C(=O)O)C(=O)O)nn5)CC4)cc3OC)c12]

(R)-2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28bRR);

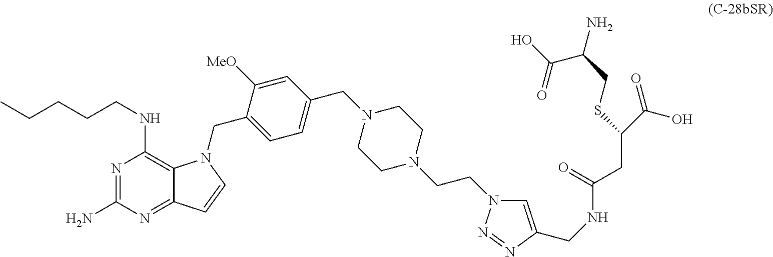 [SMILES: CCCCCNc1nc(N)nc2ccn(Cc3ccc(CN4CCN(CCn5cc(CNC(=O)C[C@H](SC[C@H](N)C(=O)O)C(=O)O)nn5)CC4)cc3OC)c12]

(S)-2-(((R)-2-amino-2-carboxyethyl)thio)-4-(((1-(2-(4-(4-((2-amino-4-(pentylamino)-5H-pyrrolo[3,2-d]pyrimidin-5-yl)methyl)-3-methoxybenzyl)piperazin-1-yl)ethyl)-1H-1,2,3-triazol-4-yl)methyl)amino)-4-oxobutanoic Acid (C-28bSR).

### Example 29

### Synthesis of N-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-3-(2,5-dioxo-2,5-dihydro-1H-pyrrol-1-yl)propanamide (C-29)

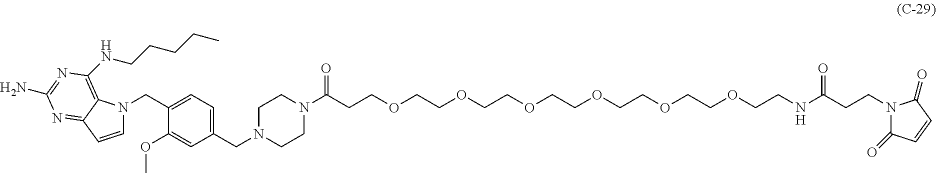 [SMILES: CCCCCNc1nc(N)nc2ccn(Cc3ccc(CN4CCN(C(=O)CCOCCOCCOCCOCCOCCOCCNC(=O)CCN5C(=O)C=CC5=O)CC4)cc3OC)c12]

N-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-3-(2,5-dioxo-2,5-dihydro-1H-pyrrol-1-yl)propanamide (C-29) was prepared following a procedure similar to Example 1, except 1-(2,5-dioxo-2,5-dihydro-1H-pyrrol-1-yl)-3-oxo-7,10,13,16,19,22-hexaoxa-4-azapentacosan-25-oic acid was used in place of 3-(2,5-dioxo-2,5-dihydro-1H-pyrrol-1-yl)propanoic acid, to afford N-(21-(4-(4-((2-amino-4-(pentylamino)-5H-pyrrolo[3,2-d]pyrimidin-5-yl)methyl)-3-methoxybenzyl)piperazin-1-yl)-21-oxo-3,6,9,12,15,18-hexaoxahenicosyl)-3-(2,5-dioxo-2,5-dihydro-1H-pyrrol-1-yl)propanamide (C-29) as a solid as the TFA salt: ¹H NMR (DMSO): δ 8.00 (t, 1H), 7.42 (d, 1H), 7.38 (s, 3H), 7.20 (s, 1H), 7.00 (s, 2H), 6.95 (s, 1H), 6.57 (s, 1H), 6.23 (d, 1H), 5.57 (s, 2H), 4.30 (s, 2H), 3.87 (s, 3H), 3.59 (m, 4H), 3.49 (m, 28H), 3.35 (t, 2H), 3.14 (m, 2H), 2.32 (m, 2H), 1.45 (m, 2H), 1.21 (m, 2H), 1.09 (m, 2H), 0.81 (t, 3H). LRMS [M+H]=924.4.

### Example 30

### Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate (C-30)

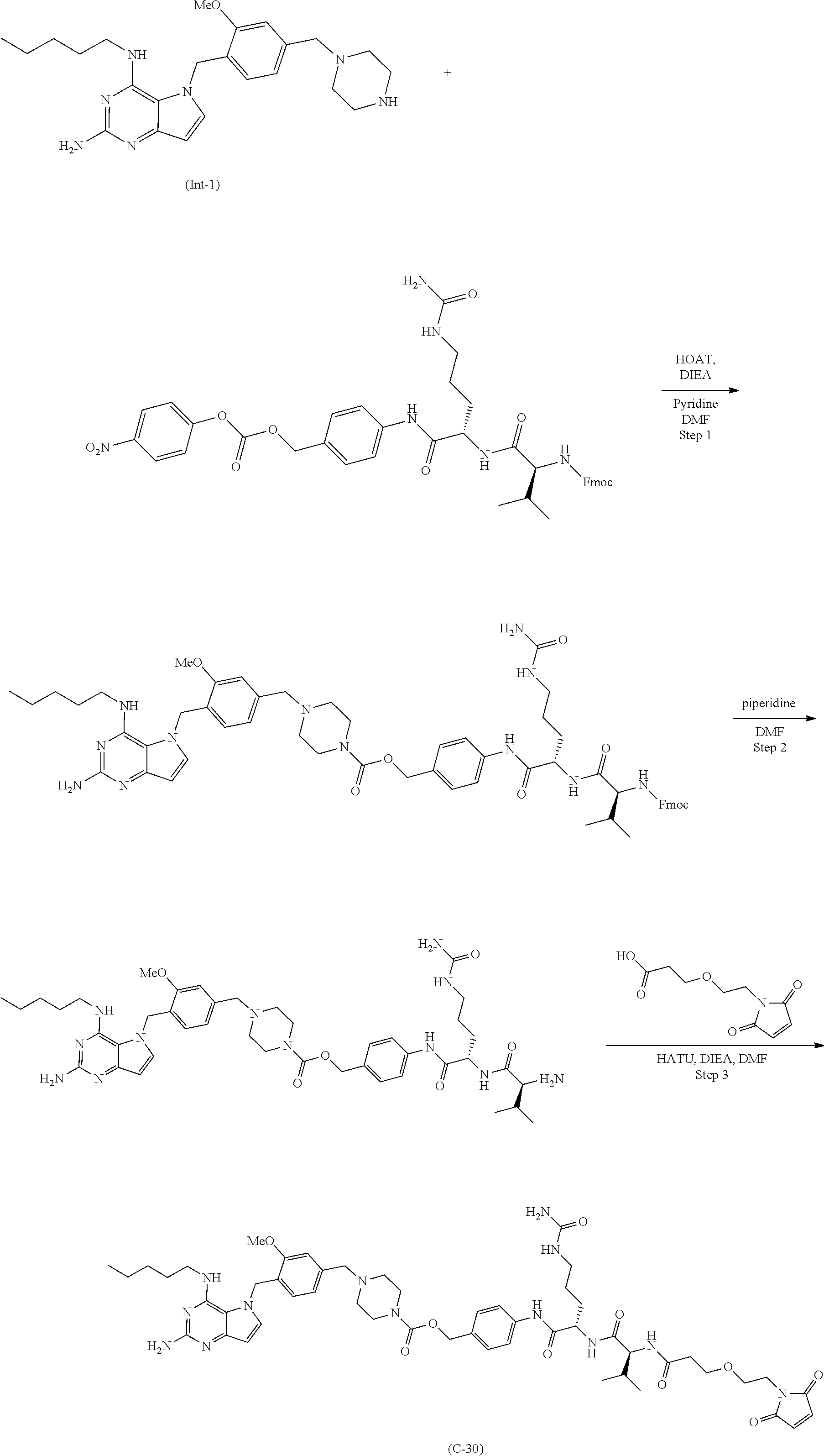

Step 1: A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), HOAT (2.0 equiv.), Huenig's base (14.0 equiv.), (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (1.2 equiv.), and pyridine:DMF (1:4, 0.02 M). The reaction mixture was stirred at room temperature for 4 hours, and the crude reaction mixture was then purified by RP-HPLC (0.035% TFA in ACN:0.05% TFA in H₂O, C18 column) to afford 4-((S)-2-((S)-2-((((9H-fluoren-9-yl)methoxy)carbonyl)amino)-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate as a solid: LCMS [M+H]=1065.5.

Step 2: 4-((S)-2-((S)-2-((((9H-fluoren-9-yl)methoxy)carbonyl)amino)-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate was dissolved in DMF (0.007 M) and piperidine (100.0 equiv.) was added. The reaction was stirred at room temperature for 30 minutes. The crude reaction mixture was then purified by RP-HPLC (0.035% TFA in ACN:0.05% TFA in H₂O, C18 column) to afford 4-((S)-2-((S)-2-amino-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate as a solid: LCMS [M+H]=843.5.

Step 3: A round bottom flask was charged with 4-((S)-2-((S)-2-amino-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate (1.0 equiv.), 3-(2-(2,5-dioxo-2,5-dihydro-1H-pyrrol-1-yl)ethoxy)propanoic acid (1.1 equiv.), Huenig's base (5.0 equiv.), HATU (1.05 equiv.) and DMF (0.004 M). The reaction mixture was stirred at room temperature for 2 hours. The crude reaction mixture was then purified by RP-HPLC (0.035% TFA in ACN:0.05% TFA in H₂O, C18 column) to afford 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl 4-(4-((2-amino-4-(pentylamino)-5H-pyrrolo[3,2-d]pyrimidin-5-yl)methyl)-3-methoxybenzyl)piperazine-1-carboxylate (C-30) as a solid as the TFA salt: LCMS [M+H]=1038.5.

### Example 31

### Synthesis of (2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2,5-dioxo-2,5-dihydro-1H-pyrrol-1-yl)propanamido)propanamido)phenoxy)-3,4,5-trihydroxytetrahydro-2H-pyran-2-carboxylic Acid (C-31)

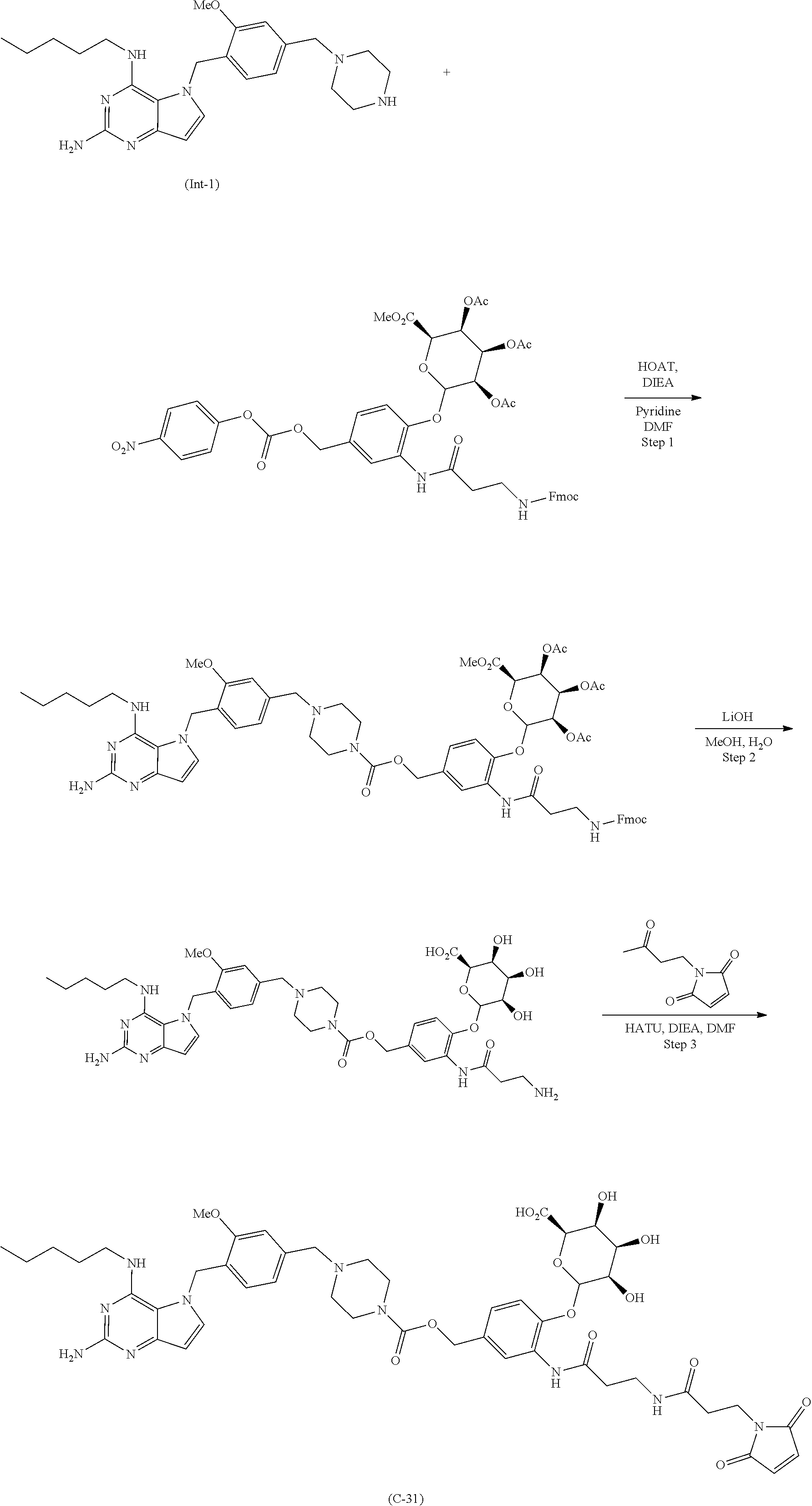

Step 1: A round bottom flask was charged with 5-(2-methoxy-4-(piperazin-1-ylmethyl)benzyl)-N4-pentyl-5H-pyrrolo[3,2-d]pyrimidine-2,4-diamine (Int-1, 1.0 equiv.), HOAT (2.0 equiv.), Huenig's base (14.0 equiv.), (3S,4R,5R,6R)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl triacetate (1.2 equiv.), and pyridine:DMF (1:4, 0.015 M). The reaction mixture was stirred at room temperature for 4 hours. The crude reaction mixture was then purified by RP-HPLC (0.035% TFA in ACN:0.05% TFA in H₂O, C18 column) to afford (3S,4R,5R,6R)-2-(2-(3-((((9H-fluoren-9-yl)methoxy)carbonyl)amino)propanamido)-4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)phenoxy)-6-(methoxycarbonyl)tetrahydro-2H-pyran-3,4,5-triyl triacetate as a solid: LCMS [M+H]=1212.4.

Step 2: (3S,4R,5R,6R)-2-(2-(3-((((9H-fluoren-9-yl)methoxy)carbonyl)amino)propanamido)-4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)phenoxy)-6-(methoxycarbonyl)tetrahydro-2H-pyran-3,4,5-triyl triacetate (1.0 equiv.) was dissolved in MeOH, THF and water (2:1:0.4) (0.005 M). LiOH (8.0 equiv.) was then added and the reaction was stirred at room temperature for 2 hours. The crude reaction mixture was then purified by RP-HPLC (0.035% TFA in ACN:0.05% TFA in H₂O, C18 column) to afford (2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-aminopropanamido)phenoxy)-3,4,5-trihydroxytetrahydro-2H-pyran-2-carboxylic acid as a solid: LCMS [M+H]=850.4.

Step 3: A round bottom flask was charged with (2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-aminopropanamido)phenoxy)-3,4,5-trihydroxytetrahydro-2H-pyran-2-carboxylic acid (1.0 equiv.), 3-(2,5-dioxo-2,5-dihydro-1H-pyrrol-1-yl)propanoic acid (2.0 equiv.), Huenig's base (6.0 equiv.), HBTU (1.8 equiv.) and DMF (0.003 M). The reaction was kept stirring at room temperature for 15 minutes. The reaction mixture was stirred at room temperature for 2 hours. The crude reaction mixture was then purified by RP-HPLC (0.035% TFA in ACN:0.05% TFA in H₂O, C18 column) to afford (2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2,5-dioxo-2,5-dihydro-1H-pyrrol-1-yl)propanamido)propanamido)phenoxy)-3,4,5-trihydroxytetrahydro-2H-pyran-2-carboxylic acid (C-31) as a solid as the TFA salt: LCMS [M+H]=1001.3.

### Example 32

### Synthesis of (S)-1-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropyl)-1H-pyrrole-2,5-dione (C-32)

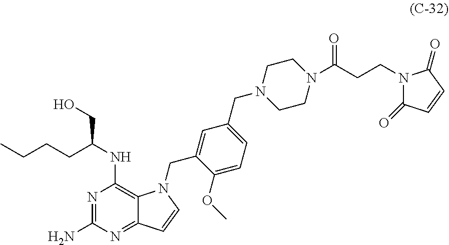 [SMILES: CCCC[C@@H](CO)Nc1nc(N)nc2ccn(Cc3cc(CN4CCN(C(=O)CCN5C(=O)C=CC5=O)CC4)ccc3OC)c12]

(S)-1-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropyl)-1H-pyrrole-2,5-dione (C-32) was prepared following a procedure similar to Example 1, except Compound (Int-2) was used in place of Compound (Int-1), to afford (S)-1-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropyl)-1H-pyrrole-2,5-dione (C-32) as a solid as the TFA salt: ¹H NMR (CD₃OD): δ 7.49 (d, 2H), 7.21 (d, 1H), 6.82 (s, 2H), 6.77 (d, 1), 6.28 (d, 1H), 5.67 (d, 1H), 5.51 (d, 1H), 4.36 (m, 1H), 4.18 (s, 2H), 3.98 (s, 3H), 3.76 (t, 2H), 3.54 (dd, 1H), 3.46 (dd, 1H), 3.16 (m, 4H), 3.05 (m, 4H), 2.71 (t, 2H), 1.48 (m, 1H), 1.26 (m, 3H), 1.05 (m, 1H), 0.84 (t, 3H). LRMS [M+H]=619.4.

### Example 33

### Synthesis of 1-(3-(4-(3-((2-amino-4-(pentylamino)-5H-pyrrolo[3,2-d]pyrimidin-5-yl)methyl)-4-methoxybenzyl)piperazin-1-yl)-3-oxopropyl)-1H-pyrrole-2,5-dione (C-33)

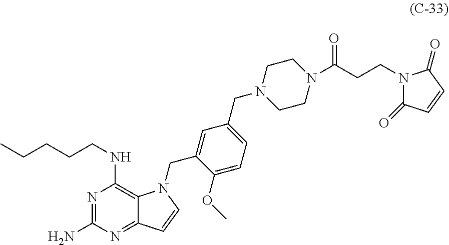 [SMILES: CCCCCNc1nc(N)nc2ccn(Cc3cc(CN4CCN(C(=O)CCN5C(=O)C=CC5=O)CC4)ccc3OC)c12]

1-(3-(4-(3-((2-amino-4-(pentylamino)-5H-pyrrolo[3,2-d]pyrimidin-5-yl)methyl)-4-methoxybenzyl)piperazin-1-yl)-3-oxopropyl)-1H-pyrrole-2,5-dione (C-33) was prepared following a procedure similar to Example 1, except Compound (Int-3) was used in place of Compound (Int-1), to afford 1-(3-(4-(3-((2-amino-4-(pentylamino)-5H-pyrrolo[3,2-d]pyrimidin-5-yl)methyl)-4-methoxybenzyl)piperazin-1-yl)-3-oxopropyl)-1H-pyrrole-2,5-dione (C-33) as a solid as the TFA salt. LRMS [M+H]=589.3.

### Example 34

### Synthesis of 3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic Acid (C-34a) and 2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic Acid (C-34b)

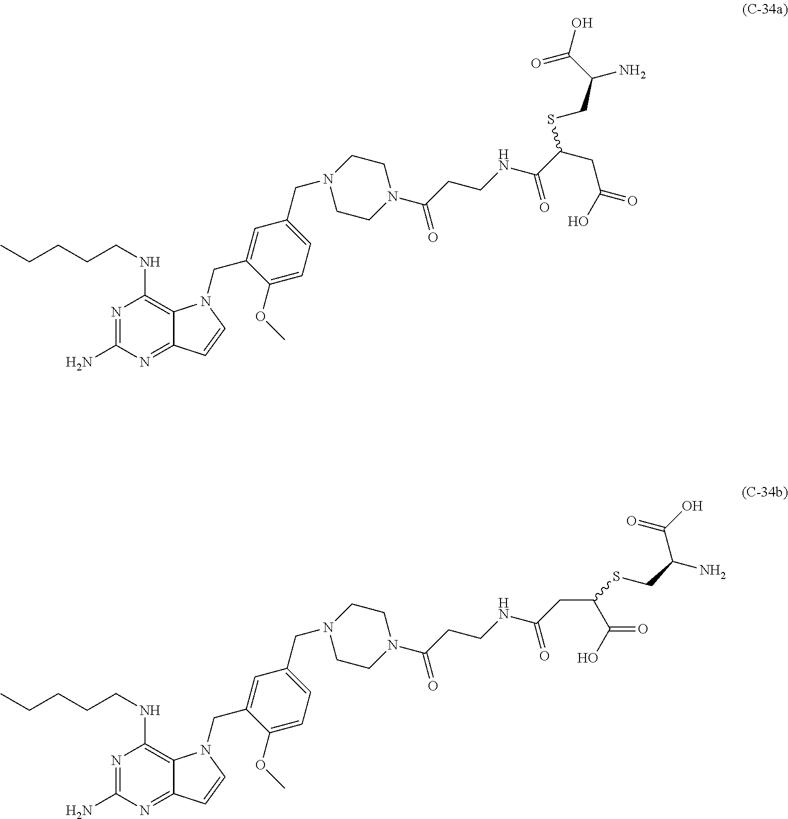 [SMILES: CCCCCNc1nc(N)nc2ccn(Cc3cc(CN4CCN(C(=O)CCNC(=O)C(CC(=O)O)SC[C@H](N)C(=O)O)CC4)ccc3OC)c12 ; CCCCCNc1nc(N)nc2ccn(Cc3cc(CN4CCN(C(=O)CCNC(=O)CC(SC[C@H](N)C(=O)O)C(=O)O)CC4)ccc3OC)c12]

3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid (C-34) and 2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic acid (C-34b) were prepared following a procedure similar to Example 4, except Compound (C-33) was used in place of Compound (C-1), to afford a mixture of Compounds (C-34a) and (C-34b), as their respective diasteromers (Compounds (C-34aSR), C-34aRR), (C-34bRR) and (C-34bRR) below), as a solid as the TFA salt. The crude reaction mixture was purified by RP-HPLC (0.035% TFA in ACN:0.05% TFA in H₂O, C18 column): ¹H NMR (DMSO): δ 7.51 (s, 2H), 7.39 (m, 2H), 7.27 (d, 1H), 7.15 (d, 1H), 6.59 (s, 1H), 6.22 (t, 1H), 5.56 (s, 2H), 3.86 (s, 4H), 3.66 (m, 3H), 3.42 (m, 8H), 3.25 (m, 4H), 3.08 (m, 2H), 2.81 (m, 3H), 2.65 (m, 1H), 1.43 (m, 2H), 1.22 (m, 3H), 1.07 (m, 2H), 0.83 (t, 3H). LCMS [M+H]=728.3

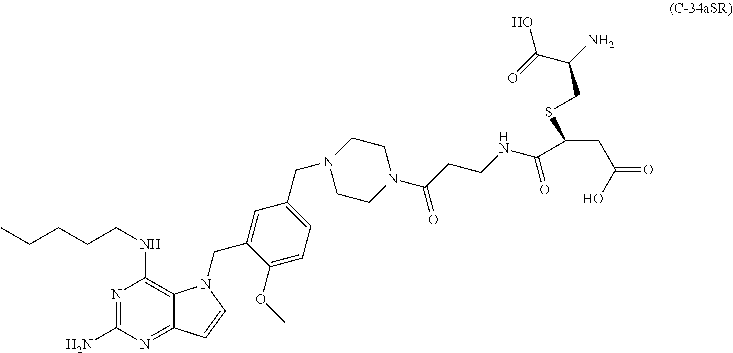 [SMILES: CCCCCNc1nc(N)nc2ccn(Cc3cc(CN4CCN(C(=O)CCNC(=O)[C@H](CC(=O)O)SC[C@H](N)C(=O)O)CC4)ccc3OC)c12]

(S)-3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic Acid (C-34aSR);

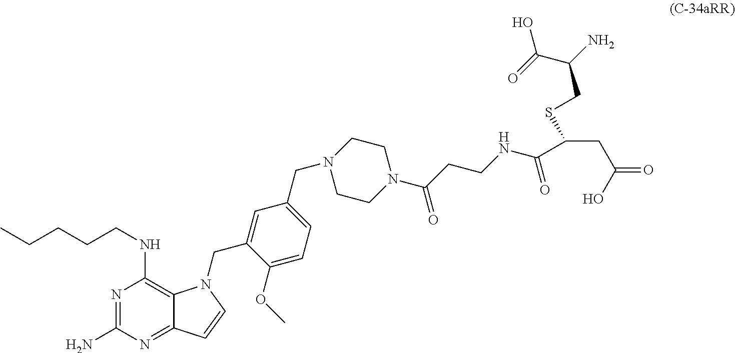 [SMILES: CCCCCNc1nc(N)nc2ccn(Cc3cc(CN4CCN(C(=O)CCNC(=O)[C@@H](CC(=O)O)SC[C@H](N)C(=O)O)CC4)ccc3OC)c12]

(R)-3-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic Acid (C-34aRR);

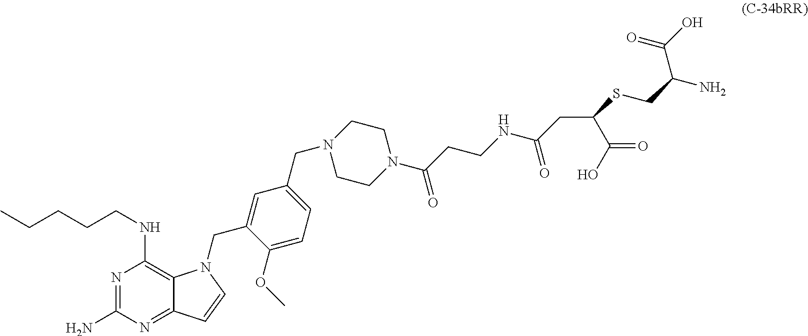 [SMILES: CCCCCNc1nc(N)nc2ccn(Cc3cc(CN4CCN(C(=O)CCNC(=O)C[C@@H](SC[C@H](N)C(=O)O)C(=O)O)CC4)ccc3OC)c12]

(R)-2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic Acid (C-34bRR);

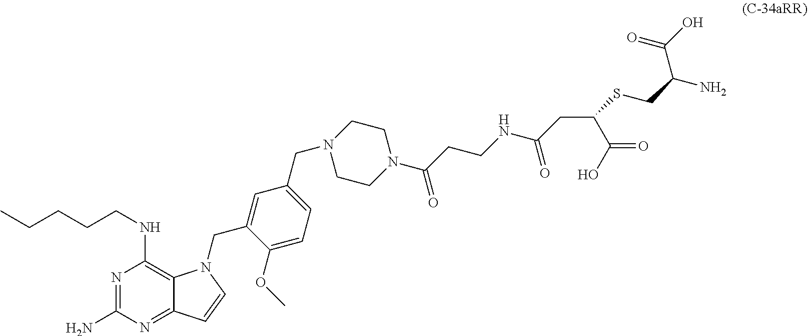 [SMILES: CCCCCNc1nc(N)nc2ccn(Cc3cc(CN4CCN(C(=O)CCNC(=O)C[C@H](SC[C@H](N)C(=O)O)C(=O)O)CC4)ccc3OC)c12]

(S)-2-(((R)-2-amino-2-carboxyethyl)thio)-4-((3-(4-(3-((2-amino-4-(pentylamino)-5H-pyrrolo[3,2-d]pyrimidin-5-yl)methyl)-4-methoxybenzyl)piperazin-1-yl)-3-oxopropyl)amino)-4-oxobutanoic Acid (C-34bSR).

### Example 35

### Synthesis of 1-(4-(4-((2-amino-4-(pentylamino)-5H-pyrrolo[3,2-d]pyrimidin-5-yl)methyl)-3-methoxybenzyl)piperazin-1-yl)-2-(aminooxy)ethanone (C-35)

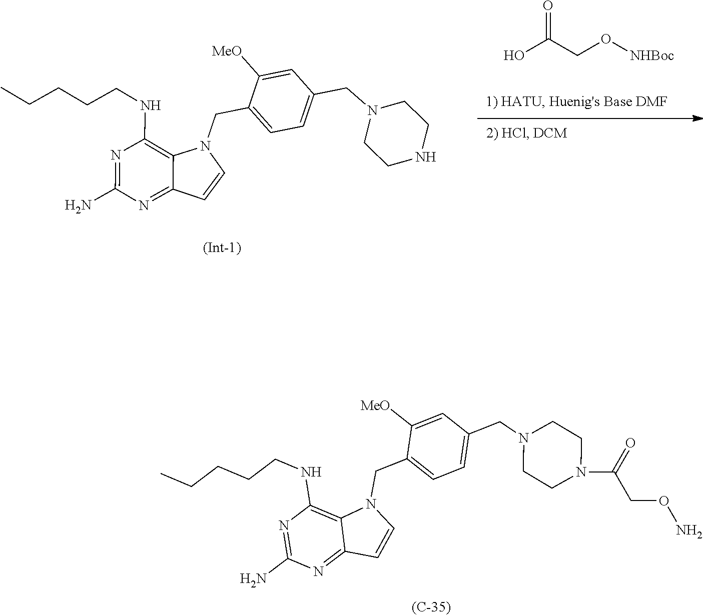 [SMILES: CCCCCNc1nc(N)nc2ccn(Cc3ccc(CN4CCNCC4)cc3OC)c12 ; CC(C)(C)OC(=O)NOCC(=O)O ; CCCCCNc1nc(N)nc2ccn(Cc3ccc(CN4CCN(C(=O)CON)CC4)cc3OC)c12 ; "(C-35)"]

Step 1: A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), 2-(((tert-butoxycarbonyl)amino)oxy)acetic acid (1.1 equiv.), HATU (1.05 equiv.), Huenig's base (5.0 equiv.), and DMF (0.2 M). The reaction mixture was stirred at room temperature for 18 hours and the crude reaction mixture was then purified by ISCO chromatography (0-20% MeOH:DCM) to provide tert-butyl 2-(4-(4-((2-amino-4-(pentylamino)-5H-pyrrolo[3,2-d]pyrimidin-5-yl)methyl)-3-methoxybenzyl)piperazin-1-yl)-2-oxoethoxycarbamate.

Step 2: HCl (20.0 equiv., 4M in dioxane) was added to a round bottom flask charged with tert-butyl 2-(4-(4-((2-amino-4-(pentylamino)-5H-pyrrolo[3,2-d]pyrimidin-5-yl)methyl)-3-methoxybenzyl)piperazin-1-yl)-2-oxoethoxycarbamate (1.0 equiv.) and DCM (0.1 M) at 0° C. The ice bath was removed and reaction mixture stirred at room temperature for 3 hours. The volatiles were removed in vacuo. MeOH (with 8% NH₄OH) was added to the resulting residue and the volatiles removed in vacuo. This was repeated 2 more times. The crude reaction mixture was then purified by ISCO chromatography (0-10% MeOH (8% NH₄OH):DCM) to deliver 1-(4-(4-((2-amino-4-(pentylamino)-5H-pyrrolo[3,2-d]pyrimidin-5-yl)methyl)-3-methoxybenzyl)piperazin-1-yl)-2-(aminooxy)ethanone (C-35) as a solid: ¹H NMR (CDCl₃): δ 7.12 (d, 1H), 7.00 (s, 1H), 6.90 (s, 1H), 6.69 (d, 1H), 6.38 (d, 1H), 5.52 (t, 1H), 5.30 (s, 2H), 4.35 (s, 2H), 3.94 (s, 3H), 3.64 (s, 2H), 3.52 (m, 2H), 3.38 (m, 4H), 2.44 (m, 4H), 1.62 (s, 2H), 1.45 (m, 2H), 1.38 (m, 2H), 1.25 (m, 2H), 1.12 (m, 2H), 0.87 (t, 3H). LRMS [M+H]=511.4.

### Example 36

### Synthesis of 1-(4-(4-((2-amino-4-(pentylamino)-5H-pyrrolo[3,2-d]pyrimidin-5-yl)methyl)-3-methoxybenzyl)piperazin-1-yl)-3-(2-aminoethoxy)propan-1-one (C-36)

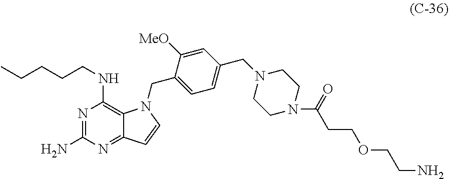 [SMILES: CCCCCNc1nc(N)nc2ccn(Cc3ccc(CN4CCN(C(=O)CCOCCN)CC4)cc3OC)c12]

1-(4-(4-((2-amino-4-(pentylamino)-5H-pyrrolo[3,2-d]pyrimidin-5-yl)methyl)-3-methoxybenzyl)piperazin-1-yl)-3-(2-aminoethoxy)propan-1-one (C-36) was prepared following a procedure similar to Example 35, except 3-(2-((tert-butoxycarbonyl)amino)ethoxy)propanoic acid was used in place of 2-(((tert-butoxycarbonyl)amino)oxy)acetic acid, to afford 1-(4-(4-((2-amino-4-(pentylamino)-5H-pyrrolo[3,2-d]pyrimidin-5-yl)methyl)-3-methoxybenzyl)piperazin-1-yl)-3-(2-aminoethoxy)propan-1-one (C-36) as a solid: ¹H NMR (CD₃OD): δ 7.26 (d, 1H), 7.09 (d, 1H), 6.86 (d, 1H), 6.59 (d, 1H), 6.13 (d, 1H), 5.43 (s, 2H), 4.57 (s, 2H), 3.94 (s, 3H), 3.73 (t, 2H), 3.58 (m, 4H), 3.54 (m, 2H), 3.37 (m, 2H), 2.93 (t, 2H), 2.66 (m, 2H), 2.44 (m, 4H), 1.41 (m, 2H), 1.27 (m, 2H), 1.15 (m, 2H), 0.87 (t, 3H). LRMS [M+H]=553.4.

### Example 37

### Synthesis of N-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-2-(aminooxy)acetamide (C-37)

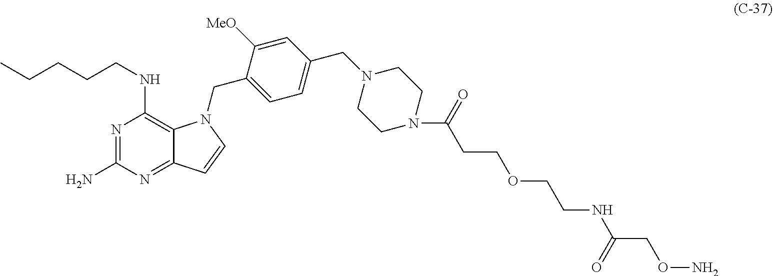 [SMILES: CCCCCNc1nc(N)nc2ccn(Cc3ccc(CN4CCN(C(=O)CCOCCNC(=O)CON)CC4)cc3OC)c12]

N-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-2-(aminooxy)acetamide (C-37) was prepared following a procedure similar to Example 35, except 1-(4-(4-((2-amino-4-(pentylamino)-5H-pyrrolo[3,2-d]pyrimidin-5-yl)methyl)-3-methoxybenzyl)piperazin-1-yl)-3-(2-aminoethoxy)propan-1-one (C-36) was used in place of Int-1, to afford N-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethyl)-2-(aminooxy)acetamide (C-37) as a solid: ¹H NMR (CD₃OD): δ 7.27 (d, 1H), 7.09 (d, 1H), 6.86 (d, 1H), 6.59 (d, 1H), 6.13 (d, 1H), 5.44 (s, 2H), 4.08 (s, 2H), 3.93 (s, 3H), 3.72 (t, 2H), 3.56 (m, 8H), 3.40 (m, 4H), 2.64 (t, 2H), 2.44 (m, 4H), 1.43 (m, 2H), 1.27 (m, 2H), 1.14 (m, 2H), 0.87 (t, 3H). LRMS [M+H]=626.4.

### Example 38

### Synthesis of (S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-2-(aminooxy)ethanone (C-38)

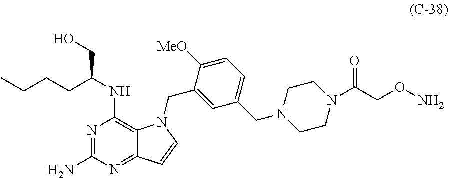 [SMILES: CCCC[C@@H](CO)Nc1nc(N)nc2ccn(Cc3cc(CN4CCN(C(=O)CON)CC4)ccc3OC)c12]

(S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-2-(aminooxy)ethanone (C-38) was prepared following a procedure similar to Example 35, except Compound (Int-2) was used in place of Compound (Int-1), to afford (S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-2-(aminooxy)ethanone (C-38) as a solid: ¹H NMR (CD₃OD): δ 7.54 (d, 1), 7.40 (d, 1H), 7.13 (d, 1H), 6.68 (s, 1H), 6.29 (d, 1H), 5.69 (d, 1H), 5.48 (d, 1H), 4.36 (m, 3H), 3.96 (s, 3H), 3.74 (m, 2H), 3.51 (m, 4H), 2.66 (m, 4H), 1.49 (m, 1H), 1.38 (m, 3H), 1.24 (m, 2H), 0.96 (m, 2H), 0.84 (t, 3H). LRMS [M+H]=541.3.

### Example 39

### (S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-(2-(2-aminoethoxy)ethoxy)propan-1-one (C-39)

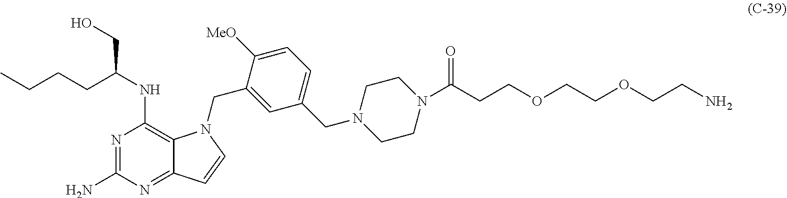 [SMILES: CCCC[C@@H](CO)Nc1nc(N)nc2ccn(Cc3cc(CN4CCN(C(=O)CCOCCOCCN)CC4)ccc3OC)c12]

(S)-1-(4-(3-((2-amino-4((1-hydroxyhexan-2-yl)amino)-5H-pyrrolo[3,2-d]pyrimidin-5-yl)methyl)-4-methoxybenzyl)piperazin-1-yl)-3-(2-(2-aminoethoxy)ethoxy)propan-1-one (C-39) was prepared following a procedure similar to Example 35, except Compound (Int-2) was used in place of Compound (Int-1) and 2,2-dimethyl-4-oxo-3,8,11-trioxa-5-azatetradecan-14-oic acid was used in place of 2-(((tert-butoxycarbonyl)amino)oxy)acetic acid, to afford (S)-1-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-(2-(2-aminoethoxy)ethoxy)propan-1-one (C-39) as a solid: ¹H NMR (CD₃OD): δ 7.56 (d, 1H), 7.44 (d, 1H), 7.16 (d, 1H), 6.77 (s, 1H), 6.31 (d, 1H), 5.71 (d, 1H), 5.50 (d, 1H), 4.38 (m, 1H), 3.98 (s, 3H), 3.78 (m, 4H), 3.72 (m, 2H), 3.67 (m, 6H), 3.53 (m, 4H), 3.14 (m, 2H), 2.77 (m, 2H), 2.69 (m, 4H), 1.51 (m, 1H), 1.26 (m, 3H), 1.02 (m, 2H), 0.86 (t, 3H). LRMS [M+H]=627.5.

### Example 40

### Synthesis of (S)—N-(2-(2-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropoxy)ethoxy)ethyl)-2-(aminooxy)acetamide (C-40)

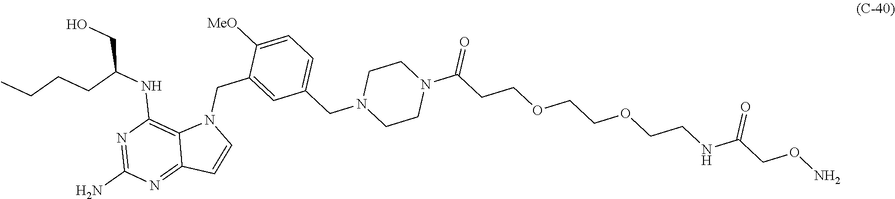 [SMILES: CCCC[C@@H](CO)Nc1nc(N)nc2ccn(Cc3cc(CN4CCN(C(=O)CCOCCOCCNC(=O)CON)CC4)ccc3OC)c12]

(S)—N-(2-(2-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropoxy)ethoxy)ethyl)-2-(aminooxy)acetamide (C-40) was prepared following a procedure similar to Example 35, except Compound (C-39) was used in place of Compound (Int-1), to afford (S)—N-(2-(2-(3-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-3-oxopropoxy)ethoxy)ethyl)-2-(aminooxy)acetamide (C-40) as a solid: ¹H NMR (CD₃OD): δ 7.54 (d, 1H), 7.47 (d, 1H), 7.17 (d, 1H), 6.78 (s, 1H), 6.30 (d, 1H), 5.68 (d, 1H), 5.50 (d, 1H), 4.36 (m, 1H), 4.09 (s, 2H), 3.97 (s, 3H), 3.73 (m, 8H), 3.56 (m, 4H), 3.43 (t, 2H), 3.23 (m, 2H), 2.88 (m, 4H), 2.66 (t, 2H), 1.49 (m, 1H), 1.26 (m, 3H), 1.04 (m, 2H), 0.84 (t, 3H). LRMS [M+H]=700.4.

### Example 41

### Synthesis of N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(aminooxy)acetamide (C-41)

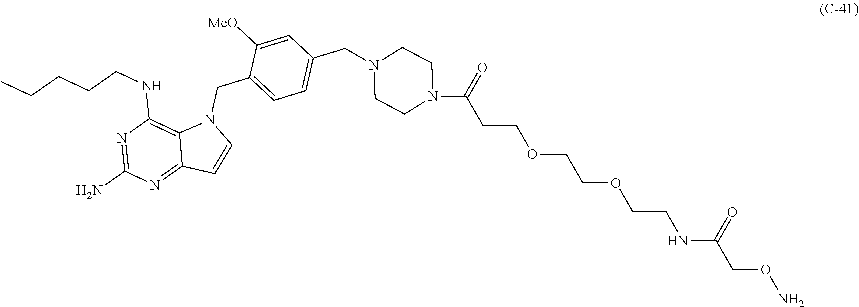 [SMILES: CCCCCNc1nc(N)nc2ccn(Cc3ccc(CN4CCN(C(=O)CCOCCOCCNC(=O)CON)CC4)cc3OC)c12]

N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(aminooxy)acetamide (C-41) was prepared following a procedure similar to Example 35, except Compound (C-20) was used in place of Compound (Int-1), to afford N-(2-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)ethyl)-2-(aminooxy)acetamide (C-41) as a solid: ¹H NMR (CD₃OD): δ 7.25 (d, 1H), 7.11 (s, 1H), 6.86 (d, 1H), 6.58 (d, 1H), 6.12 (d, 1H), 5.43 (s, 2H), 4.10 (s, 2H), 3.96 (s, 3H), 3.76 (t, 2H), 3.60 (m, 12H), 3.44 (t, 2H), 3.36 (t, 2H), 2.66 (t, 2H), 2.46 (m, 4H), 1.40 (m, 2H), 1.30 (m, 2H), 1.15 (m, 2H), 0.89 (t, 3H). LRMS [M+H]=670.4.

### Example 42

### Synthesis of 5-(4-((4-(2-(2-(aminooxy)ethoxy)ethyl)piperazin-1-yl)methyl)-2-methoxybenzyl)-N4-pentyl-5H-pyrrolo[3,2-d]pyrimidine-2,4-diamine (C-42)

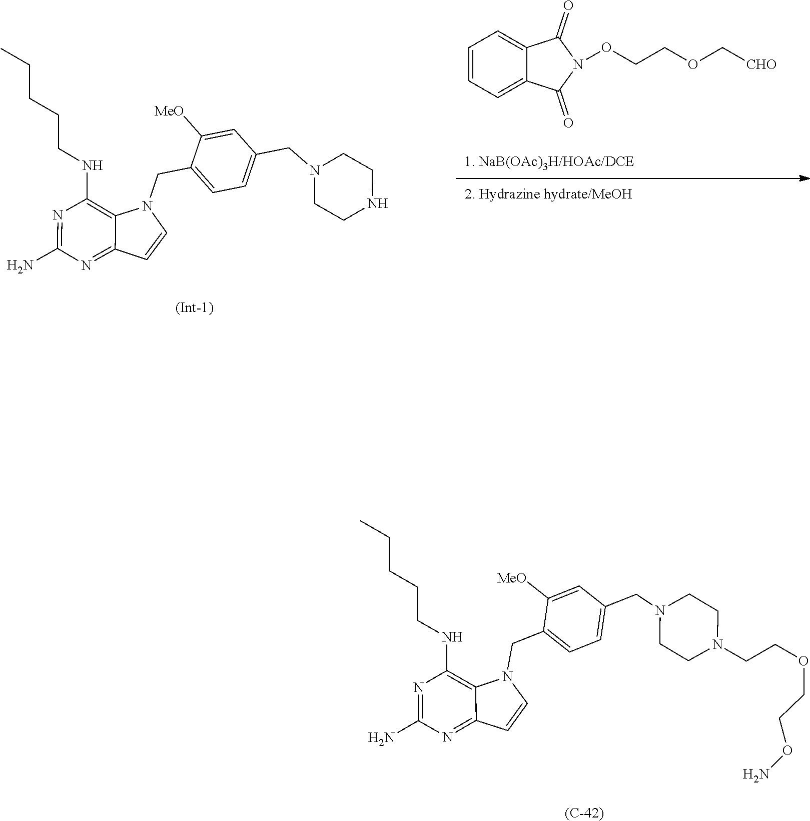 [SMILES: CCCCCNc1nc(N)nc2ccn(Cc3ccc(CN4CCNCC4)cc3OC)c12 ; O=CCOCCON1C(=O)c2ccccc2C1=O ; CCCCCNc1nc(N)nc2ccn(Cc3ccc(CN4CCN(CCOCCON)CC4)cc3OC)c12 ; "(C-42)"]

Step 1. In the first step a round bottom flask was charged with 5-(2-methoxy-4-(piperazin-1-ylmethyl)benzyl)-N4-pentyl-5H-pyrrolo[3,2-d]pyrimidine-2,4-diamine (Int-1, 1.0 equiv.) and 2-(2-((1,3-dioxoisoindolin-2-yl)oxy)ethoxy)acetaldehyde (1.2 equiv.) in DCE (0.02 M) and to this mixture was added acetic acid (6.0 equiv.), the mixture was stirred for 15 minutes at room temperature, then sodium triacetoxyborohydride (3.0 equiv.) was added. Stirring was continued for another 3 hours at room temperature. The volatiles were then removed in vacuo. The residue was dissolved in MeOH and purified by reverse phase HPLC, using C18 column (eluted with 10-50% acetonitrile-H₂O containing 0.05% TFA) to deliver 2-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethoxy)isoindoline-1,3-dione. LCMS [M+H]=671.40.

Step 2. A round bottom flask was charged with 2-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethoxy)isoindoline-1,3-dione (1.0 equiv.), hydrazine hydrate (10.0 equiv.), MeOH (0.02 M) and water (0.2 M). The mixture was stirred for 4 hours at room temperature. The reaction mixture was purified by reverse phase HPLC, using C18 column (eluted with 10-50% acetonitrile-H₂O containing 0.05% TFA). The fractions containing desired product were pooled and concentrated under reduced pressure, the residue was then dissolved in MeOH and loaded to a preconditioned Sphere PL-HCO3 MP-resin column and eluted with MeOH, the eluent was concentrated to afford 5-(4-((4-(2-(2-(aminooxy)ethoxy)ethyl)piperazin-1-yl)methyl)-2-methoxybenzyl)-N4-pentyl-5H-pyrrolo[3,2-d]pyrimidine-2,4-diamine (C-42) as a solid: ¹H NMR (CD₃OD): δ 7.22 (d, 1H), 7.08 (d, 1H), 6.83 (d, 1H), 6.56 (d, 1H), 6.10 (d, 1H), 5.40 (s, 2H), 3.94 (s, 3H), 3.76 (m, 2H), 3.60 (m, 4H), 3.50 (s, 2H), 3.34 (d, 3H), 2.59 (m, 4H), 2.49 (s, 4H), 1.38 (m, 2H), 1.26 (m, 2H), 1.12 (m, 2H), 0.87 (t, 3H). LCMS [M+H]=541.40.

Note: 2-(2-((1,3-dioxoisoindolin-2-yl)oxy)ethoxy)acetaldehyde was prepared in a two step process:

Step 1: To a solution of N-hydroxyphthalimide (1.0 equiv.), diethylene glycol (1.0 equiv.) and triphenylphosphine (1.3 equiv.) in THF (0.2 M) was added DEAD (2.2 M solution in toluene, 1.3 equiv.) at 0° C. The resulting solution was stirred overnight at room temperature. The reaction mixture was concentrated in vacuo. The residue was purified by silica gel chromatography (eluted with 20-70% EtOA/Hexanes). The product still contained some Ph₃PO after this chromatography, it was then repurified by reverse phase chromatography (C18 column, eluted with 20-40-100% CH₃CN/water) to afford 2-(2-(2-hydroxyethoxy)ethoxy)isoindoline-1,3-dione LCMS [M+H]=252.10.

Step 2: To a stirred mixture of 2-(2-(2-hydroxyethoxy)ethoxy)isoindoline-1,3-dione (1.0 equiv.) and sodium bicarbonate (2.0 equiv.) in dry DCM (0.08 M) was added Dess-Martin periodinane (2.0 equiv.), the resulting mixture was stirred for 3 hours at room temperature. The reaction mixture was diluted with DCM, then washed with 1N NaOH solution and brine, the organic layer was separated and dried over MgSO4 and evaporated in vacuo. The crude mixture was purified by silica gel chromatography (eluted with 30-70% EtAOc/Hexanes), to deliver 2-(2-((1,3-dioxoisoindolin-2-yl)oxy)ethoxy)acetaldehyde. LCMS [M+H]=250.10.

### Example 43

### Synthesis of N-(3-(4-(4-((2-amino-4-(pentylamino)-5H-pyrrolo[3,2-d]pyrimidin-5-yl)methyl)-3-methoxybenzyl)piperazin-1-yl)propyl)-2-(aminooxy)acetamide (C-43)

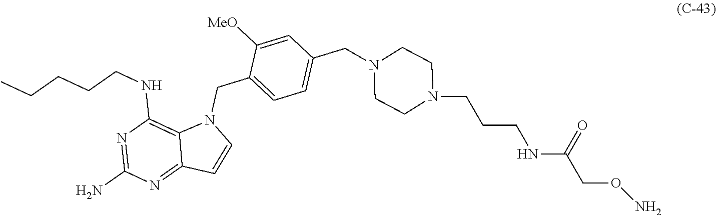 [SMILES: CCCCCNc1nc(N)nc2ccn(Cc3ccc(CN4CCN(CCCNC(=O)CON)CC4)cc3OC)c12]

N-(3-(4-(4-((2-amino-4-(pentylamino)-5H-pyrrolo[3,2-d]pyrimidin-5-yl)methyl)-3-methoxybenzyl)piperazin-1-yl)propyl)-2-(aminooxy)acetamide (C-43) was prepared following a procedure similar to Example 35, except Compound (C-19) was used in place of Compound (Int-1), to afford N-(3-(4-(4-((2-amino-4-(pentylamino)-5H-pyrrolo[3,2-d]pyrimidin-5-yl)methyl)-3-methoxybenzyl)piperazin-1-yl)propyl)-2-(aminooxy)acetamide (C-43) as a solid: ¹H NMR (CD₃OD): δ 7.12 (d, 1H), 6.98 (d, 1H), 6.73 (d, 1H), 6.45 (d, 1H), 6.00 (d, 1H), 5.30 (s, 2H), 3.97 (s, 2H), 3.84 (s, 3H), 3.41 (s, 2H), 3.25 (s, 2H), 2.40 (s, 6H), 2.27 (m, 3H), 1.63 (m, 2H), 1.28 (m, 2H), 1.17 (m, 3H), 1.02 (m, 2H), 0.77 (t, 3H). LCMS [M+H]=568.40.

### Example 44

### Synthesis of 5-(4-((4-(2-(2-(2-aminoethoxy)ethoxy)ethyl) piperazin-1-yl)methyl)-2-methoxybenzyl)-N4-pentyl-5H-pyrrolo[3,2-d]pyrimidine-2,4-diamine (C-44)

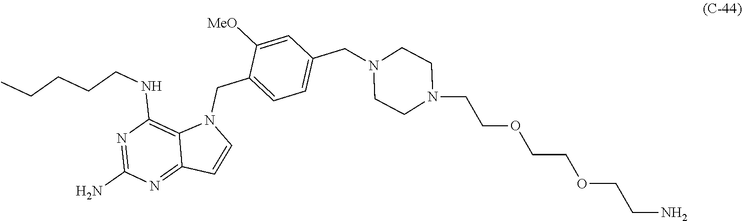 [SMILES: CCCCCNc1nc(N)nc2ccn(Cc3ccc(CN4CCN(CCOCCOCCN)CC4)cc3OC)c12]

5-(4-((4-(2-(2-(2-aminoethoxy)ethoxy)ethyl)piperazin-1-yl)methyl)-2-methoxybenzyl)-N4-pentyl-5H-pyrrolo[3,2-d]pyrimidine-2,4-diamine (C-44) was prepared following a procedure similar to Example 19, except tert-butyl (2-(2-(2-bromoethoxy)ethoxy)ethyl)carbamate was used in place tert-butyl (3-bromopropyl)carbamate, to afford 5-(4-((4-(2-(2-(2-aminoethoxy)ethoxy)ethyl)piperazin-1-yl)methyl)-2-methoxybenzyl)-N4-pentyl-5H-pyrrolo[3,2-d]pyrimidine-2,4-diamine (C-44) as a solid: ¹H NMR (CD₃OD): δ 7.36 (d, 1H), 7.13 (d, 1H), 6.92 (d, 1H), 6.73 (d, 1H), 6.21 (s, 1H), 5.51 (s, 2H), 3.92 (s, 3H), 3.69 (m, 12H), 3.53 (t, 2H), 3.12 (m, 2H), 2.84 (m, 8H), 1.50 (m, 2H), 1.28 (m, 2H), 1.17 (m, 2H), 0.87 (t, 3H). LRMS [M+H]=569.3.

### Example 45

### Synthesis of N-(2-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethoxy)ethyl)-2-(aminooxy)acetamide (C-45)

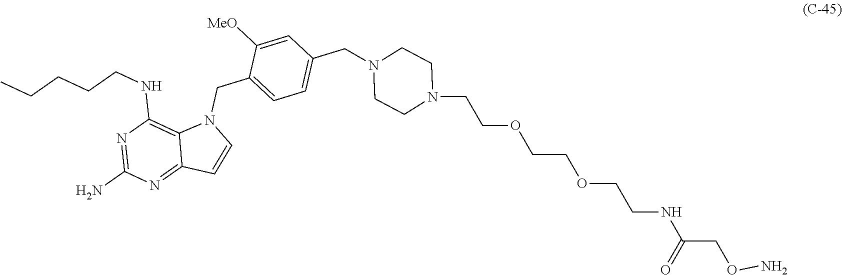 [SMILES: CCCCCNc1nc(N)nc2ccn(Cc3ccc(CN4CCN(CCOCCOCCNC(=O)CON)CC4)cc3OC)c12]

N-(2-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethoxy)ethyl)-2-(aminooxy)acetamide (C-43) was prepared following a procedure similar to Example 35, except Compound (C-44) was used in place of Compound (Int-1), to afford N-(2-(2-(2-(4-(4-((2-amino-4-(pentylamino)-5H-pyrrolo[3,2-d]pyrimidin-5-yl)methyl)-3-methoxybenzyl)piperazin-1-yl)ethoxy)ethoxy)ethyl)-2-(aminooxy)acetamide (C-45) as a solid: ¹H NMR (CDCl₃): δ 7.20 (s, 1H), 6.97 (d, 1H), 6.90 (s, 1H), 6.87 (s, 1H), 6.76 (d, 1H), 6.56 (d, 1H), 6.17 (d, 1H), 5.84 (s, 2H), 5.21 (s, 2H), 4.69 (m, 2H), 4.07 (s, 2H), 3.85 (s, 3H), 3.53 (m, 8H), 3.45 (m, 2H), 3.39 (s, 2H), 3.24 (m, 2H), 2.52 (t, 2H), 2.40 (m, 8H), 1.22 (m, 2H), 1.16 (m, 2H), 1.02 (m, 2H), 0.78 (t, 3H). LRMS [M+H]=642.4.

### Example 46

### Synthesis of 2,5-dioxopyrrolidin-1-yl 5-(4-(4-((2-amino-4-(pentylamino)-5H-pyrrolo[3,2-d]pyrimidin-5-yl)methyl)-3-methoxybenzyl)piperazin-1-yl)-5-oxopentanoate (C-46)

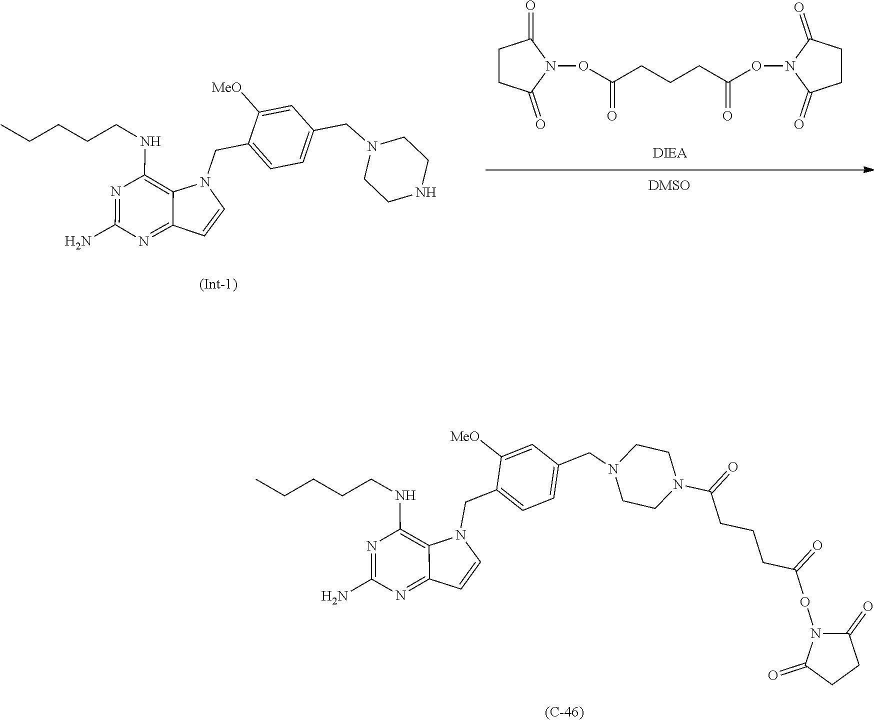 [SMILES: CCCCCNc1nc(N)nc2ccn(Cc3ccc(CN4CCNCC4)cc3OC)c12 ; O=C(CCCC(=O)ON1C(=O)CCC1=O)ON1C(=O)CCC1=O ; CCCCCNc1nc(N)nc2ccn(Cc3ccc(CN4CCN(C(=O)CCCC(=O)ON5C(=O)CCC5=O)CC4)cc3OC)c12 ; "(C-46)"]

A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), diisopropyl amine (1.3 equiv.), disuccinimidal glutarate (1.3 equiv.), and DMSO (0.1 M). The reaction mixture was stirred room temperature for 3 hours. The crude reaction mixture was then purified by RP-HPLC (0.035% TFA in ACN:0.05% TFA in H₂O, C18 column) to afford 2,5-dioxopyrrolidin-1-yl 5-(4-(4-((2-amino-4-(pentylamino)-5H-pyrrolo[3,2-d]pyrimidin-5-yl)methyl)-3-methoxybenzyl)piperazin-1-yl)-5-oxopentanoate (C-46) as a solid as the TFA salt: ¹H NMR (DMSO): δ 7.41 (s, 1H), 7.37 (s, 3H), 7.19 (s, 1H), 6.94 (s, 1H), 6.57 (s, 1H), 6.22 (d, 1H), 5.56 (s, 2H), 4.30 (s, 2H), 3.86 (s, 3H), 3.44 (m, 4H), 3.35 (m, 2H), 2.92 (m, 2H), 2.80 (m, 8H), 2.71 (m, 2H), 1.83 (m, 2H), 1.44 (m, 2H), 1.20 (m, 2H), 1.09 (m, 2H), 0.80 (t, 3H). LRMS [M+H]=649.3.

### Example 47

### Synthesis of (S)-2,5-dioxopyrrolidin-1-yl 5-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-5-oxopentanoate (C-47)

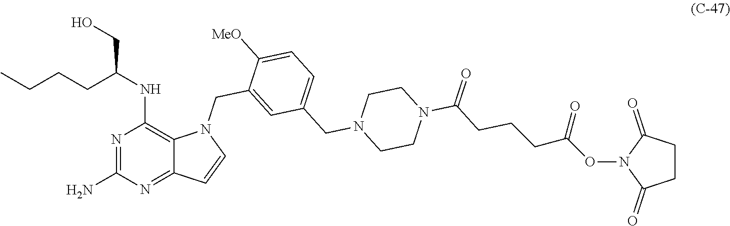 [SMILES: CCCC[C@@H](CO)Nc1nc(N)nc2ccn(Cc3cc(CN4CCN(C(=O)CCCC(=O)ON5C(=O)CCC5=O)CC4)ccc3OC)c12]

(S)-2,5-dioxopyrrolidin-1-yl 5-(4-(3-((2-amino-4-((1-hydroxyhexan-2-yl)amino-5H-pyrrolo[3,2-d]pyrimidin-5-yl)methyl)-4-methoxybenzyl)piperazin-1-yl)-5-oxopentanoate (C-47) was prepared following a procedure similar to Example 46, except Compound (Int-2) was used in place of Compound (Int-1), to afford (S)-2,5-dioxopyrrolidin-1-yl 5-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-5-oxopentanoate (C-47) as a solid as the TFA salt: ¹H NMR (DMSO): δ 7.54 (s, 1H), 7.43 (s, 3H), 7.22 (s, 1H), 6.61 (s, 1H), 6.28 (d, 1H), 6.24 (d, 1H), 5.67 (d, 1H), 5.50 (d, 1H), 4.82 (s, 1H), 4.39 (s, 1H), 4.22 (m, 2H), 3.89 (s, 3H), 3.36 (m, 4H), 3.28 (m, 2H), 2.92 (m, 2H), 2.82 (m, 8H), 2.72 (m, 2H), 1.84 (m, 2H), 1.34 (m, 2H), 1.15 (m, 2H), 0.86 (m, 2H), 0.77 (t, 3H). LRMS [M+H]=679.3.

### Example 48

### Synthesis of (S)-2-amino-6-(5-(4-(3-((2-amino-4-(((S)-1-hydroxyhexan-2-yl)amino)-5H-pyrrolo[3,2-d]pyrimidin-5-yl)methyl)-4-methoxybenzyl)piperazin-1-yl)-5-oxopentanamido)hexanoic Acid (C-48)

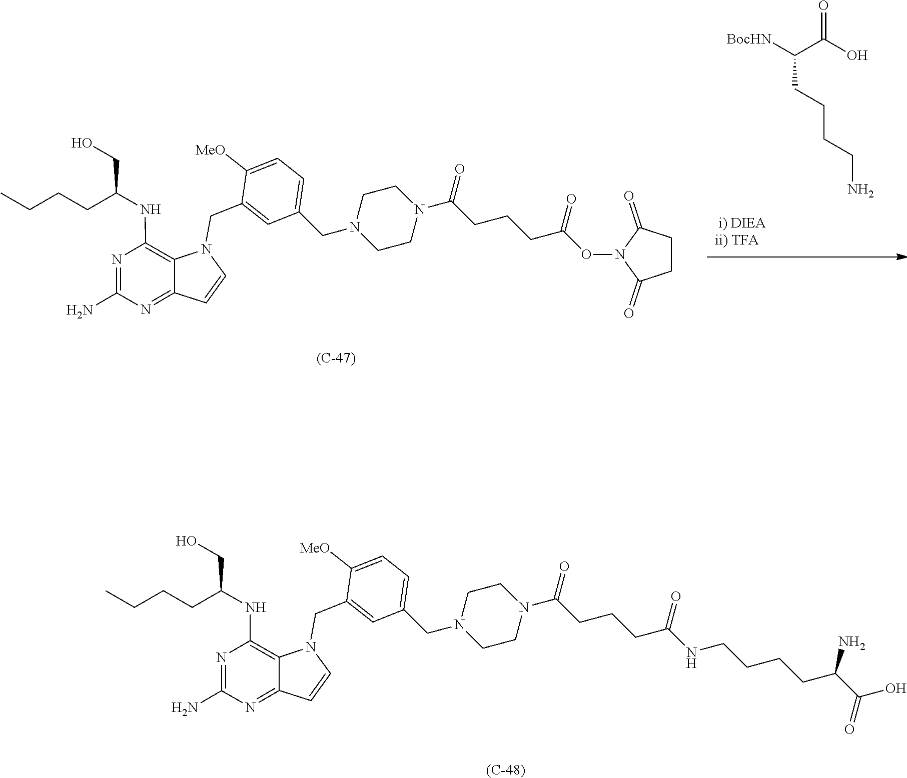 [SMILES: CCCC[C@@H](CO)Nc1nc(N)nc2ccn(Cc3cc(CN4CCN(C(=O)CCCC(=O)ON5C(=O)CCC5=O)CC4)ccc3OC)c12 ; CC(C)(C)OC(=O)N[C@@H](CCCCN)C(=O)O ; "(C-47)" ; CCCC[C@@H](CO)Nc1nc(N)nc2ccn(Cc3cc(CN4CCN(C(=O)CCCC(=O)NCCCC[C@@H](N)C(=O)O)CC4)ccc3OC)c12 ; "(C-48)"]

A round bottom flask was charged with (S)-2,5-dioxopyrrolidin-1-yl 5-(4-(3-((2-amino-4-((1-hydroxyhexan-2-yl)amino)-5H-pyrrolo[3,2-d]pyrimidin-5-yl)methyl)-4-methoxybenzyl)piperazin-1-yl)-5-oxopentanoate (0-47 (1.0 eq), Boc-Lys-OH (2.0 eq), DIEA (5.0 eq) and DMF (30 mM). The reaction was stirred at room temperature for 16 hours and the volatiles were removed in vacuo. The crude reaction mixture was purified using RP-HPLC (0.035% TFA in ACN:0.05% TFA in H₂O, 018 column) to obtain (S)-6-(5-(4-(3-((2-amino-4-(((S)-1-hydroxyhexan-2-yl)amino)-5H-pyrrolo[3,2-d]pyrimidin-5-yl)methyl)-4-methoxybenzyl)piperazin-1-yl)-5-oxopentanamido)-2-((tert-butoxycarbonyl)amino)hexanoic acid LCMS [M+1]=810.5. (S)-6-(5-(4-(3-((2-amino-4-(((S)-1-hydroxyhexan-2-yl)amino)-5H-pyrrolo[3,2-d]pyrimidin-5-yl)methyl)-4-methoxybenzyl)piperazin-1-yl)-5-oxopentanamido)-2-((tert-butoxycarbonyl)amino)hexanoic acid was treated with 30% TFA by volume in 0.1 M DCM and the volatiles removed in vacuo to obtain (S)-2-amino-6-(5-(4-(3-((2-amino-4-(((S)-1-hydroxyhexan-2-yl)amino)-5H-pyrrolo[3,2-d]pyrimidin-5-yl)methyl)-4-methoxybenzyl)piperazin-1-yl)-5-oxopentanamido)hexanoic acid (C-48) as a solid as the TFA salt: ¹H NMR (00300): δ 7.49 (m, 2H), 7.21 (d, 1H), 6.77 (s, 1H), 6.29 (d, 1H), 5.68 (d, 1H), 5.50 (d, 1H), 4.36 (m, 1H), 4.20 (m, 2H), 3.99 (S, 3H), 3.93 (m, 1H), 3.76 (m, 2H), 3.50 (m, 2H), 3.19 (m, 4H), 2.44 (t, 2H), 2.24 (t, 2H), 2.16 (m, 4H), 1.88 (m, 4H), 1.51 (m, 2H), 1.25 (m, 6H), 1.03 (m, 2H), 0.84 (t, 3H). LRMS [M+H]=710.3.

### Example 49

### Synthesis of (S)-2-amino-6-(5-(4-(4-((2-amino-4-(pentylamino)-5H-pyrrolo[3,2-d]pyrimidin-5-yl)methyl)-3-methoxybenzyl)piperazin-1-yl)-5-oxopentanamido)hexanoic Acid (0-49)

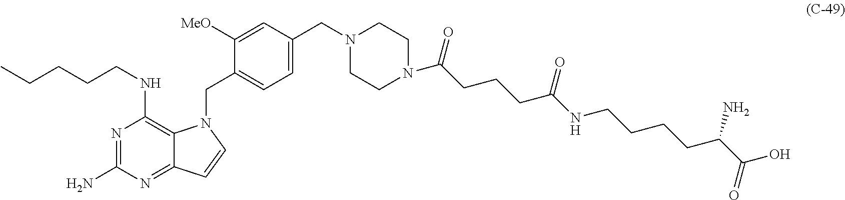 [SMILES: CCCCCNc1nc(N)nc2ccn(Cc3ccc(CN4CCN(C(=O)CCCC(=O)NCCCC[C@H](N)C(=O)O)CC4)cc3OC)c12]

(S)-2-amino-6-(5-(4-(4-((2-amino-4-(pentylamino)-5H-pyrrolo[3,2-d]pyrimidin-5-yl)methyl)-3-methoxybenzyl)piperazin-1-yl)-5-oxopentanamido)hexanoic Acid (0-49) was prepared following a procedure similar to Example 48, except Compound (0-46) was used in place of Compound (0-47), to afford (S)-2-amino-6-(5-(4-(4-((2-amino-4-(pentylamino)-5H-pyrrolo[3,2-d]pyrimidin-5-yl)methyl)-3-methoxybenzyl)piperazin-1-yl)-5-oxopentanamido)hexanoic Acid (C-49) as a solid as the TFA salt: ¹H NMR (CD₃OD): δ 7.37 (d, 1H), 7.22 (d, 1H), 7.01 (d, 1H), 6.78 (d, 1H), 6.23 (s, 1H), 5.56 (s, 2H), 4.07 (m, 2H), 3.95 (s, 3H), 3.79 (m, 1H), 3.73 (m, 2H), 3.55 (m, 2H), 2.98 (m, 4H), 2.43 (t, 2H), 2.23 (t, 2H), 2.04 (m, 4H), 1.89 (m, 4H), 1.54 (m, 6H), 1.30 (m, 2H), 1.19 (m, 2H), 0.88 (t, 3H). LRMS [M+H]=680.4.

### Example 50

### Synthesis of 2,5-dioxopyrrolidin-1-yl 5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanoate (C-50)

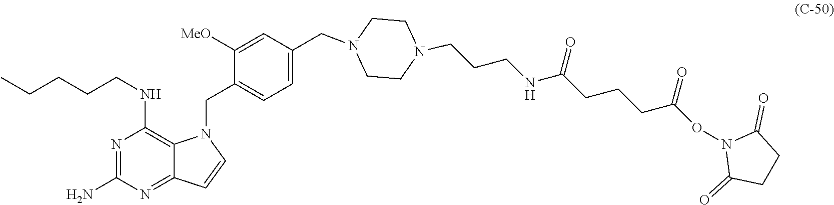 [SMILES: CCCCCNc1nc(N)nc2ccn(Cc3ccc(CN4CCN(CCCNC(=O)CCCC(=O)ON5C(=O)CCC5=O)CC4)cc3OC)c12]

2,5-dioxopyrrolidin-1-yl 5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanoate (C-50) was prepared following a procedure similar to Example 46, except Compound (C-19) was used in place of Compound (Int-1), to afford 2,5-dioxopyrrolidin-1-yl 5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanoate (C-50) as a solid as the TFA salt: ¹H NMR (DMSO): δ 8.00 (s, 1H), 7.40 (m, 4H), 7.02 (s, 1H), 6.82 (s, 1H), 6.55 (d, 1H), 6.21 (d, 1H), 5.53 (s, 2H), 3.83 (m, 5H), 3.00 (m, 8H), 2.81 (m, 4H), 2.69 (m, 2H), 2.19 (m, 2H), 1.84 (m, 2H), 1.75 (m, 4H), 1.45 (m, 2H), 1.22 (m, 4H), 1.09 (m, 4H), 0.80 (t, 3H). LRMS [M+H]=706.4.

### Example 51

### Synthesis of (S)-2-amino-6-(5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanamido)hexanoic Acid (C-51)

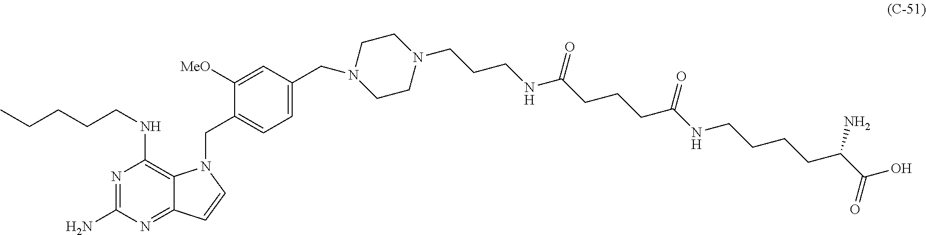 [SMILES: CCCCCNc1nc(N)nc2ccn(Cc3ccc(CN4CCN(CCCNC(=O)CCCC(=O)NCCCC[C@H](N)C(=O)O)CC4)cc3OC)c12]

(S)-2-amino-6-(5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanamido)hexanoic acid (C-51) was prepared following a procedure similar to Example 48, except Compound (C-50) was used in place of Compound (C-47), to afford (S)-2-amino-6-(5-((3-(4-(4-((2-amino-4-(pentylamino)-5H-pyrrolo[3,2-d]pyrimidin-5-yl)methyl)-3-methoxybenzyl)piperazin-1-yl)propyl)amino)-5-oxopentanamido)hexanoic acid (C-51) as a solid as the TFA salt: ¹H NMR (CD₃OD): δ 7.35 (d, 1H), 7.12 (s, 1H), 6.94 (d, 1H), 6.75 (d, 1H), 6.22 (s, 1H), 5.52 (s, 2H), 3.92 (s, 3H), 3.86 (t, 1H), 3.71 (s, 2H), 3.54 (m, 2H), 3.22 (m, 8H), 3.05 (m, 2H), 2.82 (m, 2H), 2.21 (m, 4H), 1.89 (m, 4H), 1.53 (m, 6H), 1.30 (m, 4H), 1.18 (m, 2H), 0.88 (t, 3H). LRMS [M+H]=737.4.

### Example 52

### Synthesis of 2,5-dioxopyrrolidin-1-yl 5-(4-(3-((2-amino-4-(pentylamino)-5H-pyrrolo[3,2-d]pyrimidin-5-yl)methyl)-4-methoxybenzyl)piperazin-1-yl)-5-oxopentanoate (C-52)

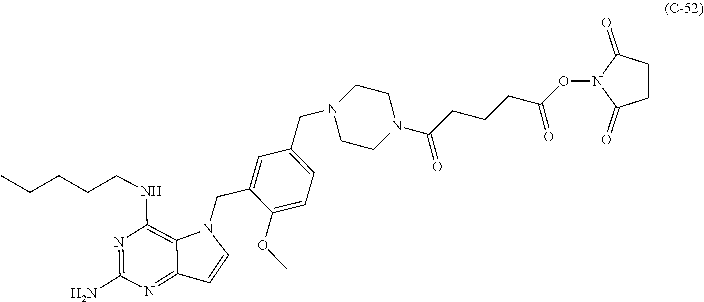 [SMILES: CCCCCNc1nc(N)nc2ccn(Cc3cc(CN4CCN(C(=O)CCCC(=O)ON5C(=O)CCC5=O)CC4)ccc3OC)c12]

2,5-dioxopyrrolidin-1-yl 5-(4-(3-((2-amino-4-(pentylamino)-5H-pyrrolo[3,2-d]pyrimidin-5-yl)methyl)-4-methoxybenzyl)piperazin-1-yl)-5-oxopentanoate (C-52) was prepared following a procedure similar to Example 46, except Compound (Int-3) was used in place of Compound (Int-1), to afford 2,5-dioxopyrrolidin-1-yl 5-(4-(3-((2-amino-4-(pentylamino)-5H-pyrrolo[3,2-d]pyrimidin-5-yl)methyl)-4-methoxybenzyl)piperazin-1-yl)-5-oxopentanoate (C-52) as a solid as the TFA salt: LRMS [M+H]=649.4.

### Example 53

### Synthesis of (S)-2-amino-6-(5-(4-(3-((2-amino-4-(pentylamino)-5H-pyrrolo[3,2-d]pyrimidin-5-yl)methyl)-4-methoxybenzyl)piperazin-1-yl)-5-oxopentanamido)hexanoic Acid (C-53)

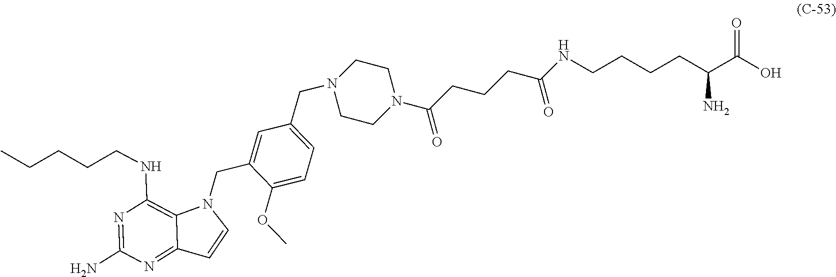 [SMILES: CCCCCNc1nc(N)nc2ccn(Cc3cc(CN4CCN(C(=O)CCCC(=O)NCCCC[C@H](N)C(=O)O)CC4)ccc3OC)c12]

(S)-2-amino-6-(5-(4-(3-((2-amino-4-(pentylamino)-5H-pyrrolo[3,2-d]pyrimidin-5-yl)methyl)-4-methoxybenzyl)piperazin-1-yl)-5-oxopentanamido)hexanoic acid (C-53) was prepared following a procedure similar to Example 48, except Compound (C-52) was used in place of Compound (C-47), to afford S)-2-amino-6-(5-(4-(3-((2-amino-4-(pentylamino)-5H-pyrrolo[3,2-d]pyrimidin-5-yl)methyl)-4-methoxybenzyl)piperazin-1-yl)-5-oxopentanamido)hexanoic acid (C-53) as a solid as the TFA salt: ¹H NMR (DMSO): δ 8.22 (s, 3H), 7.79 (t, 1H), 7.51 (s, 2H), 7.42 (m, 2H), 7.27 (t, 1H), 7.17 (d, 1H), 6.61 (s, 1H), 6.23 (d, 1H), 5.57 (s, 2H), 4.05 (m, 2H), 3.87 (s, 5H), 3.42 (m, 3H), 3.02 (m, 3H), 2.89 (m, 2H), 2.31 (t, 2H), 2.09 (t, 2H), 1.72 (m, 4H), 1.41 (m, 5H), 1.22 (m, 2H), 1.07 (m, 2H), 0.83 (t, 3H). LRMS [M+H]=680.4.

### Example 54

### Synthesis of perfluorophenyl 5-(4-(4-((2-amino-4-(pentylamino)-5H-pyrrolo[3,2-d]pyrimidin-5-yl)methyl)-3-methoxybenzyl)piperazin-1-yl)-5-oxopentanoate (C-54)

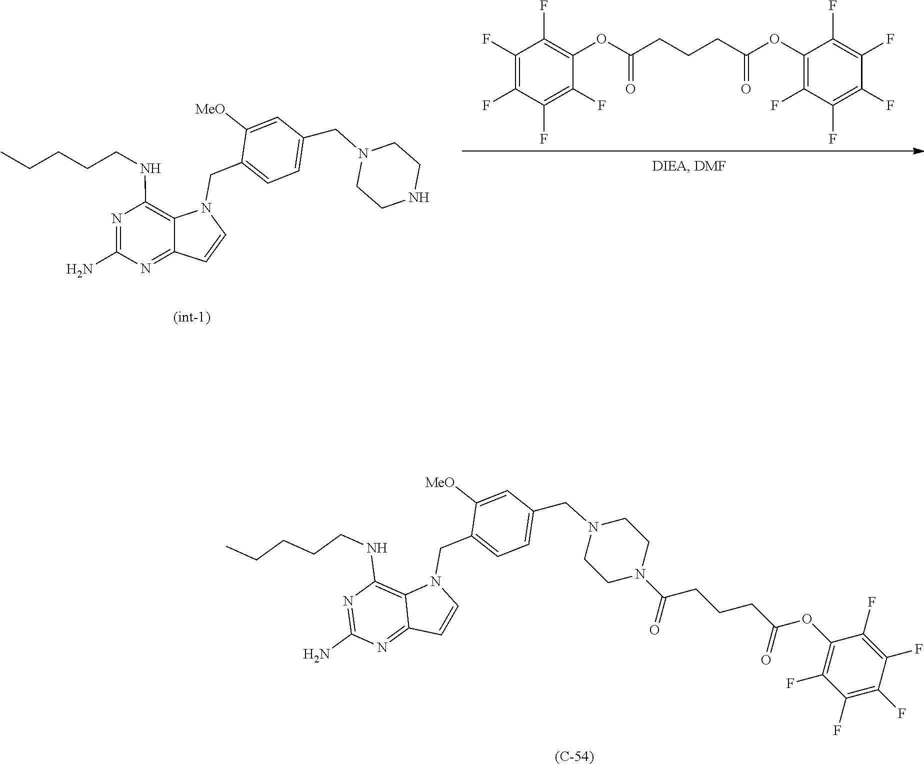 [SMILES: CCCCCNc1nc(N)nc2ccn(Cc3ccc(CN4CCNCC4)cc3OC)c12 ; O=C(CCCC(=O)Oc1c(F)c(F)c(F)c(F)c1F)Oc1c(F)c(F)c(F)c(F)c1F ; "(int-1)" ; CCCCCNc1nc(N)nc2ccn(Cc3ccc(CN4CCN(C(=O)CCCC(=O)Oc5c(F)c(F)c(F)c(F)c5F)CC4)cc3OC)c12 ; "(C-54)"]

A round-bottom flask was charged with 5-(2-methoxy-4-(piperazin-1-ylmethyl)benzyl)-N4-pentyl-5H-pyrrolo[3,2-d]pyrimidine-2,4-diamine (Int-1, 1.0 equiv.), DIEA (3.0 equiv.), bis(perfluorophenyl) glutarate (2.0 equiv.), and DMF (0.01 M). The reaction was stirred at room temperature for 2 hours and then the crude reaction mixture was purified by RP-HPLC (0.035% TFA in ACN:0.05% TFA in H₂O, C18 column) yielding perfluorophenyl 5-(4-(4-((2-amino-4-(pentylamino)-5H-pyrrolo[3,2-d]pyrimidin-5-yl)methyl)-3-methoxybenzyl)piperazin-1-yl)-5-oxopentanoate (C-54) as a solid as the TFA salt. LCMS [M+1]=718.4.

Note: Bis(perfluorophenyl) glutarate was prepared by glutaroyl dichloride (1.0 equiv.), THF (0.15 M) and triethylamine (2.2 equiv.) to a round bottom flask and cooling the reaction mixture to 0° C. A solution of 2,3,4,5,6-pentafluorophenol (2.1 equiv.) in THF (1.2 M) was then added slowly. The reaction mixture was stirred for 2 hours at room temperature. The mixture was filtered through silica gel and then concentrated in vacuo. The residue was purified by silica gel column eluted with hexane-ethyl acetate (9:1) and concentrated to give bis(perfluorophenyl) glutarate as solid. LCMS [M+23]=487.2.

### Example 55

### Synthesis of perfluorophenyl 3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanoate (C-55)

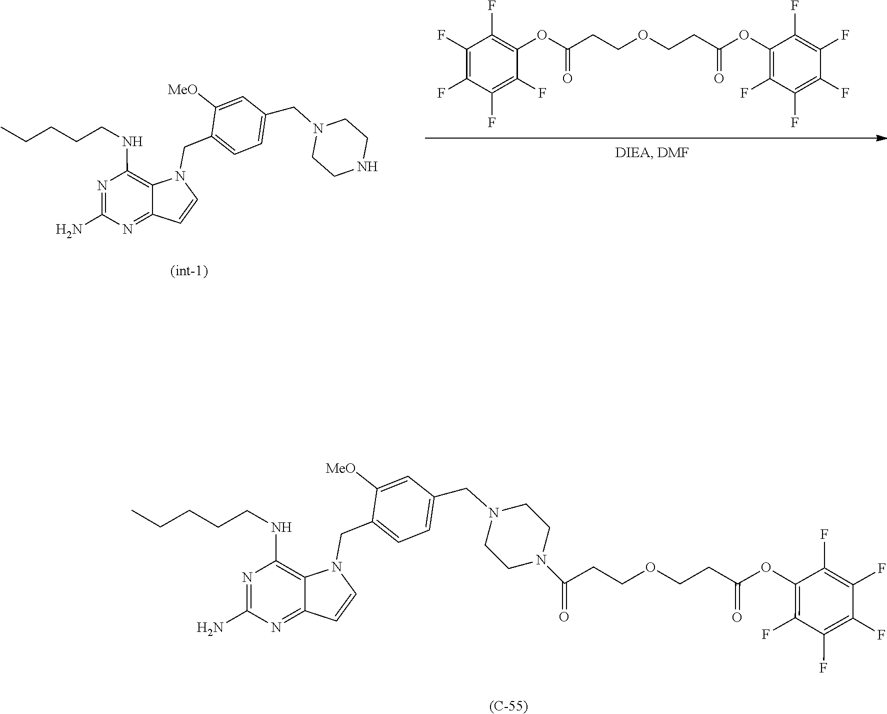 [SMILES: CCCCCNc1nc(N)nc2ccn(Cc3ccc(CN4CCNCC4)cc3OC)c12 ; O=C(CCOCCC(=O)Oc1c(F)c(F)c(F)c(F)c1F)Oc1c(F)c(F)c(F)c(F)c1F ; "(int-1)" ; CCCCCNc1nc(N)nc2ccn(Cc3ccc(CN4CCN(C(=O)CCOCCC(=O)Oc5c(F)c(F)c(F)c(F)c5F)CC4)cc3OC)c12 ; "(C-55)"]

Perfluorophenyl 3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanoate (C-55) was prepared as a solid as the TFA salt following a procedure similar to Example 54, except bis(perfluorophenyl) 3,3′-oxydipropanoate was used in place of bis(perfluorophenyl) glutarate. ¹H NMR (Acetonitrile-d₃) δ 7.33 (d, 1H), 7.30 (d, 1H), 6.95 (d, 1H), 6.73 (d, 1H), 6.22 (d, 1H), 6.06 (m, 1H), 5.43 (s, 2H), 4.18 (s, 2H), 3.92 (s, 3H), 3.81 (t, 2H), 3.74 (t, 2H), 3.47 (m, 2H), 2.95 (t, 2H), 2.60 (t, 2H), 2.14 (d, 2H), 1.45 (m, 2H), 1.28 (m, 2H), 1.15 (m, 2H), 0.87 (t, 3H). LRMS [M+H]=748.4. ¹⁹F NMR (471 MHz, Acetonitrile-ds) 6-154.71 (d, 2F), −160.40 (d, 1F), −164.57 (dd, 2F).

### Example 56

### Synthesis of perfluorophenyl 3-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)propanoate (C-56)

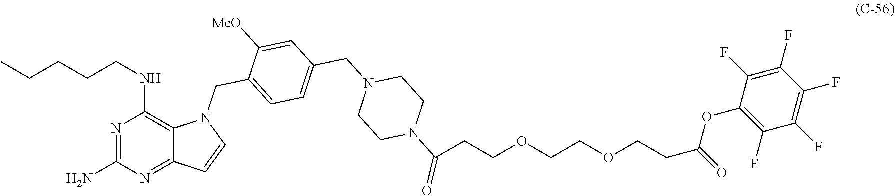 [SMILES: CCCCCNc1nc(N)nc2ccn(Cc3ccc(CN4CCN(C(=O)CCOCCOCCC(=O)Oc5c(F)c(F)c(F)c(F)c5F)CC4)cc3OC)c12]

3-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)propanoate (C-56) was prepared following a procedure similar to Example 54, except bis(perfluorophenyl) 3,3′-(ethane-1,2-diylbis(oxy))dipropanoate was used in place of bis(perfluorophenyl) glutarate to obtain 3-(2-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)ethoxy)propanoate (C-54). LRMS [M+H]=792.4.

### Example 57

### Synthesis of (S)-2-amino-6-(3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanamido)hexanoic Acid (C-57)

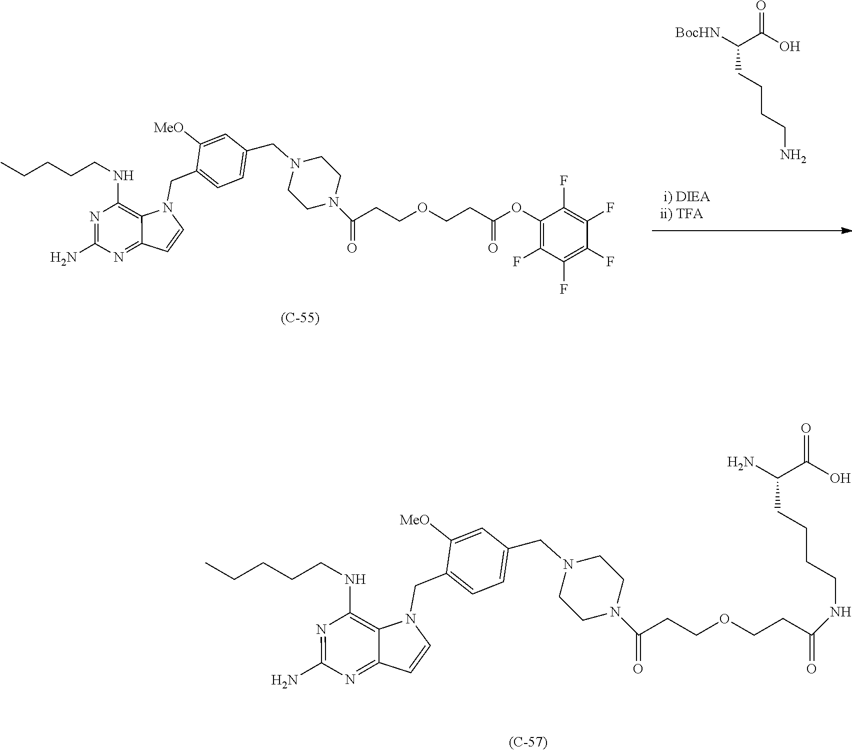 [SMILES: CCCCCNc1nc(N)nc2ccn(Cc3ccc(CN4CCN(C(=O)CCOCCC(=O)Oc5c(F)c(F)c(F)c(F)c5F)CC4)cc3OC)c12 ; CC(C)(C)OC(=O)N[C@@H](CCCCN)C(=O)O ; "(C-55)" ; CCCCCNc1nc(N)nc2ccn(Cc3ccc(CN4CCN(C(=O)CCOCCC(=O)NCCCC[C@H](N)C(=O)O)CC4)cc3OC)c12 ; "(C-57)"]

A round bottom flask was charged with perfluorophenyl 3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanoate (C-55, 1.0 equiv.), Boc-Lys-OH (2.0 equiv.), DIEA (5.0 equiv.) and DMF (30 mM). The reaction was stirred at room temperature for 16 hours and the volatiles were removed in vacuo. The crude reaction mixture was purified using RP-HPLC (0.035% TFA in ACN:0.05% TFA in H₂O, C18 column) to obtain (S)-6-(3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanamido)-2-((tert-butoxycarbonyl)amino)hexanoic acid. LCMS [M+1]=810.5. The boc protected compound was treated with 30% TFA by volume in 0.1M DCM and then the volatiles removed in vacuo to obtain (S)-2-amino-6-(3-(3-(4-(4-((2-amino-4-(pentylamino)-5H-pyrrolo[3,2-d]pyrimidin-5-yl)methyl)-3-methoxybenzyl)piperazin-1-yl)-3-oxopropoxy)propanamido)hexanoic acid (C-57) as a solid as the TFA salt: ¹H NMR (DMSO): δ 8.18 (m, 3H), 7.80 (s, 1H), 7.41 (m, 4H), 7.18 (s, 1H), 6.94 (d, 1H), 6.59 (d, 1H), 6.22 (d, 1H), 5.56 (s, 2H), 4.24 (m, 1H), 3.86 (m, 7H), 3.56 (m, 4H), 3.44 (m, 4H), 3.01 (m, 4H), 2.60 (m, 2H), 2.28 (m, 2H), 1.74 (m, 2H), 1.45 (m, 2H), 1.38 (m, 3H), 1.21 (m, 3H), 1.09 (m, 2H), 0.80 (t, 3H). LCMS [M+1]=710.5.

### Example 58

### Synthesis of N-(15-(4-(4-((2-amino-4-(pentylamino)-5H-pyrrolo[3,2-d]pyrimidin-5-yl)methyl)-3-methoxybenzyl)piperazin-1-yl)-15-oxo-3,6,9,12-tetraoxapentadecyl)-5-((3aS,4S,6aR)-2-oxohexahydro-1H-thieno[3,4-d]imidazol-4-yl)pentanamide (C-58)

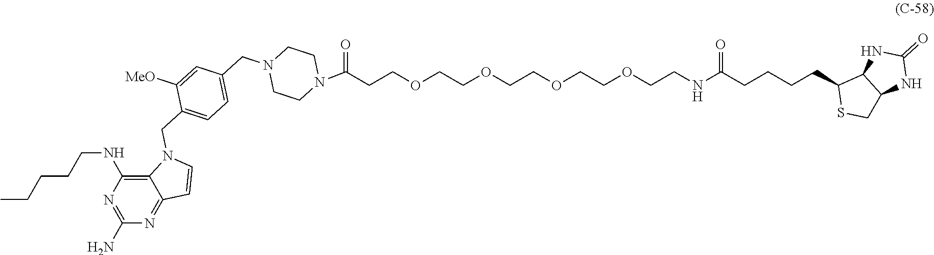 [SMILES: CCCCCNc1nc(N)nc2ccn(Cc3ccc(CN4CCN(C(=O)CCOCCOCCOCCOCCNC(=O)CCCC[C@@H]5SC[C@@H]6NC(=O)N[C@@H]65)CC4)cc3OC)c12]

N-(15-(4-(4-((2-amino-4-(pentylamino)-5H-pyrrolo[3,2-d]pyrimidin-5-yl)methyl)-3-methoxybenzyl)piperazin-1-yl)-15-oxo-3,6,9,12-tetraoxapentadecyl)-5-((3aS,4S,6aR)-2-oxohexahydro-1H-thieno[3,4-d]imidazol-4-yl)pentanamide (C-58) was prepared following a procedure similar to Example 46, except 2,5-dioxopyrrolidin-1-yl 17-oxo-21-((3aS,4S,6aR)-2-oxohexahydro-1H-thieno[3,4-d]imidazol-4-yl)-4,7,10,13-tetraoxa-16-azahenicosan-1-oate was used in place of disuccinimidal glutarate, to afford N-(15-(4-(4-((2-amino-4-(pentylamino)-5H-pyrrolo[3,2-d]pyrimidin-5-yl)methyl)-3-methoxybenzyl)piperazin-1-yl)-15-oxo-3,6,9,12-tetraoxapentadecyl)-5-((3aS,4S,6aR)-2-oxohexahydro-1H-thieno[3,4-d]imidazol-4-yl)pentanamide (C-58) as a solid as the TFA salt: ¹H NMR (DMSO): δ 7.84 (m, 2H), 7.42 (m, 4H), 7.22 (m, 1H), 6.94 (d, 1H), 6.56 (d, 1H), 6.42 (s, 1H), 6.37 (s, 1H), 6.22 (s, 1H), 5.57 (s, 2H), 4.29 (m, 2H), 4.11 (m, 2H), 3.86 (s, 3H), 3.60 (m, 4H), 3.48 (m, 16H), 3.37 (m, 4H), 3.16 (m, 4H), 3.08 (m, 2H), 2.80 (m, 1H), 2.56 (m, 2H), 2.05 (m, 2H), 1.58 (m, 1H), 1.45 (m, 5H), 1.23 (m, 4H), 1.07 (m, 2H), 0.80 (t, 3H). LRMS [M+H]=911.6.

### Example 59

### Synthesis of 4-((R)-6-amino-2-((S)-2-(3-(2,5-dioxo-2,5-dihydro-1H-pyrrol-1-yl)propanamido)-3-phenylpropanamido)hexanamido)benzyl 4-(4-((2-amino-4-(pentylamino)-5H-pyrrolo[3,2-d]pyrimidin-5-yl)methyl)-3-methoxybenzyl)piperazine-1-carboxylate (C-59)

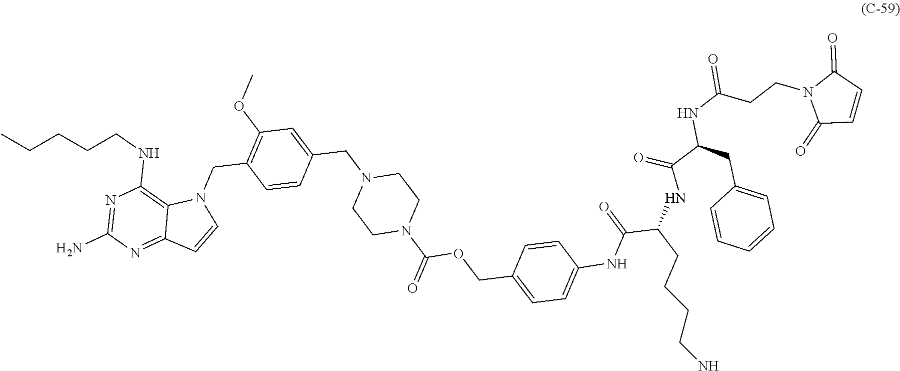 [SMILES: CCCCCNc1nc(N)nc2ccn(Cc3ccc(CN4CCN(C(=O)OCc5ccc(NC(=O)[C@@H](CCCCN)NC(=O)[C@H](Cc6ccccc6)NC(=O)CCN6C(=O)C=CC6=O)cc5)CC4)cc3OC)c12]

4-((R)-6-amino-2-((S)-2-(3-(2,5-dioxo-2,5-dihydro-1H-pyrrol-1-yl)propanamido)-3-phenylpropanamido)hexanamido)benzyl 4-(4-((2-amino-4-(pentylamino)-5H-pyrrolo[3,2-d]pyrimidin-5-yl)methyl)-3-methoxybenzyl)piperazine-1-carboxylate (C-59) was prepared as a solid as the TFA salt according to the scheme shown for Example (C-30), except (9H-fluoren-9-yl)methyl ((S)-1-(((R)-6-amino-1-((4-((((4-nitrophenoxy)carbonyl)oxy)methyl)phenyl)amino)-1-oxohexan-2-yl)amino)-1-oxo-3-phenylpropan-2-yl)carbamate was used in place of (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate in the first step: ¹H NMR (CD₃OD): δ 8.26 (d, 1H), 7.91 (t, 1H), 7.61 (d, 2H), 7.35 (m, 3H), 7.25 (m, 3H), 7.19 (m, 3H), 7.03 (d, 1H), 6.79 (d, 1H), 6.76 (s, 2H), 6.24 (d, 1H), 5.57 (s, 2H), 5.11 (s, 2H), 4.41 (m, 1H), 4.33 (s, 2H), 3.98 (t, 1H), 3.95 (s, 3H), 3.70 (m, 3H), 3.54 (t, 2H), 3.24 (m, 4H), 3.10 (m, 1H), 3.02 (m, 1H), 2.83 (m, 1H), 2.47 (t, 2H), 1.92 (m, 2H), 1.52 (m, 4H), 1.42 (m, 2H), 1.30 (m, 3H), 1.18 (m, 2H), 0.88 (t, 3H). LRMS [M+H]=1013.5.

### Example 60

### Synthesis of 4-((S)-2-((S)-2-(3-(2-(2,5-dioxo-2,5-dihydro-1H-pyrrol-1-yl)ethoxy)propanamido)-3-methylbutanamido)propanamido)benzyl 4-(4-((2-amino-4-(pentylamino)-5H-pyrrolo[3,2-d]pyrimidin-5-yl)methyl)-3-methoxybenzyl)piperazine-1-carboxylate (C-60)

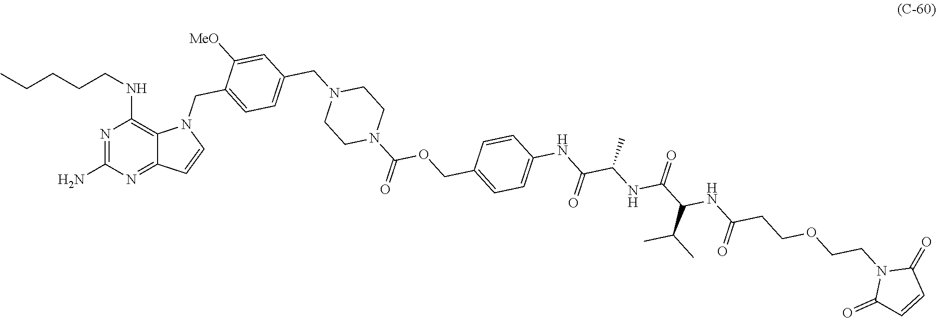

4-((S)-2-((S)-2-(3-(2-(2,5-dioxo-2,5-dihydro-1H-pyrrol-1-yl)ethoxy)propanamido)-3-methylbutanamido)propanamido)benzyl 4-(4-((2-amino-4-(pentylamino)-5H-pyrrolo[3,2-d]pyrimidin-5-yl)methyl)-3-methoxybenzyl)piperazine-1-carboxylate (C-60) was prepared as a solid as the TFA salt according to the scheme shown for Example (C-30), except (9H-fluoren-9-yl)methyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate was used in place of (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate in the first step: ¹H NMR (CD₃OD): δ 9.65 (s, 1H), 8.20 (d, 1H), 7.97 (d, 1H), 7.60 (m, 2H), 7.34 (m, 2H), 7.31 (s, 1H), 7.22 (d, 1H), 7.03 (d, 1H), 6.80 (m, 2H), 6.77 (s, 2H), 6.23 (d, 1H), 5.57 (s, 2H), 5.11 (s, 2H), 4.48 (t, 1H), 4.31 (s, 3H), 4.15 (t, 1H), 3.95 (m, 4H), 3.68 (m, 4H), 3.62 (m, 2H), 3.53 (m, 8H), 2.49 (t, 2H), 2.11 (m, 1H), 1.52 (m, 2H), 1.44 (d, 3H), 1.28 (m, 2H), 1.18 (m, 2H), 0.98 (m, 6H), 0.87 (t, 3H). LRMS [M+H]=952.6.

### Example 61

### Synthesis of (2S,3S,4S,5R,6S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2,5-dioxo-2,5-dihydro-1H-pyrrol-1-yl)propanamido)propanamido)phenoxy)-3,4,5-trihydroxytetrahydro-2H-pyran-2-carboxylic Acid (C-61)

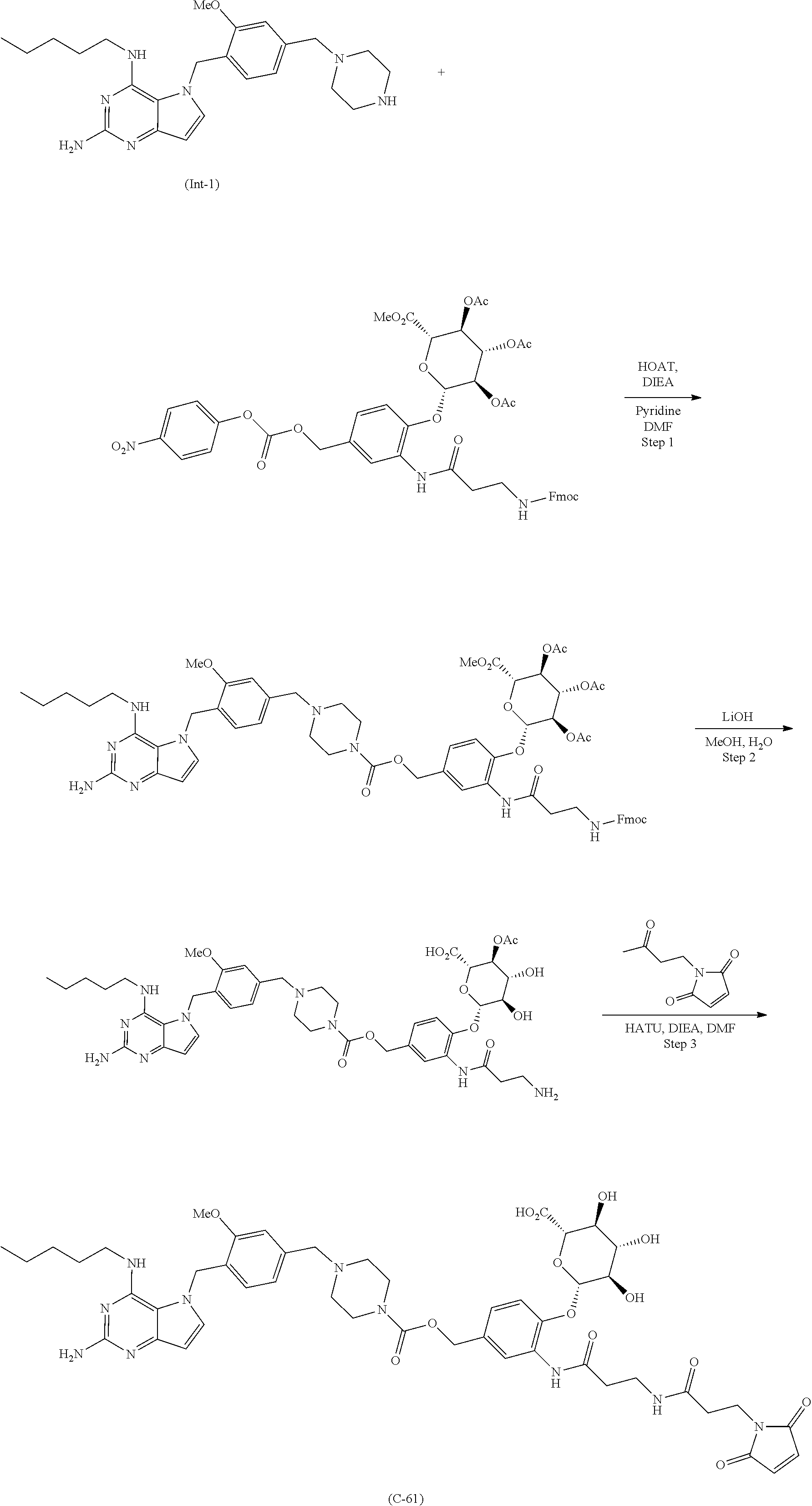

Step 1: A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), HOAT (2.0 equiv.), Huenig's base (14.0 equiv.), (3S,4R,5R,6R)-2-(2-(3-((((9H-fluoren-9-yl)methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl triacetate (1.2 equiv.), and pyridine:DMF (1:4, 0.015 M). The reaction mixture was stirred at room temperature for 4 hours. The crude reaction mixture was then purified by RP-HPLC (0.035% TFA in ACN:0.05% TFA in H₂O, C18 column) to afford (3S,4R,5R,6R)-2-(2-(3-((((9H-fluoren-9-yl)methoxy)carbonyl)amino)propanamido)-4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)phenoxy)-6-(methoxycarbonyl)tetrahydro-2H-pyran-3,4,5-triyl triacetate as a solid: LCMS [M+H]=1212.4.

Step 2: (3S,4R,5R,6R)-2-(2-(3-((((9H-fluoren-9-yl)methoxy)carbonyl)amino)propanamido)-4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)phenoxy)-6-(methoxycarbonyl)tetrahydro-2H-pyran-3,4,5-triyl triacetate (1.0 equiv.) was dissolved in MeOH, THF and water (2:1:0.4) (0.005 M). LiOH (8.0 equiv.) was then added and the reaction was stirred at room temperature for 2 hours. The crude reaction mixture was then purified by RP-HPLC (0.035% TFA in ACN:0.05% TFA in H₂O, C18 column) to afford (2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-aminopropanamido)phenoxy)-3,4,5-trihydroxytetrahydro-2H-pyran-2-carboxylic acid as a solid: LCMS [M+H]=850.4.

Step 3: A round bottom flask was charged with (2R,3R,4R,5S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-aminopropanamido)phenoxy)-3,4,5-trihydroxytetrahydro-2H-pyran-2-carboxylic acid (1.0 equiv.), 3-(2,5-dioxo-2,5-dihydro-1H-pyrrol-1-yl)propanoic acid (2.0 equiv.), Huenig's base (6.0 equiv.), HBTU (1.8 equiv.) and DMF (0.003 M). The reaction was kept stirring at room temperature for 15 minutes. The reaction mixture was stirred at room temperature for 2 hours. The crude reaction mixture was then purified by RP-HPLC (0.035% TFA in ACN:0.05% TFA in H₂O, C18 column) to afford (2S,3S,4S,5R,6S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2,5-dioxo-2,5-dihydro-1H-pyrrol-1-yl)propanamido)propanamido)phenoxy)-3,4,5-trihydroxytetrahydro-2H-pyran-2-carboxylic acid (C-61) as a solid as the TFA salt: LCMS [M+H]=1001.3.

### Example 62

### Synthesis of (2S,3S,4S,5R,6S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic Acid (C-62)

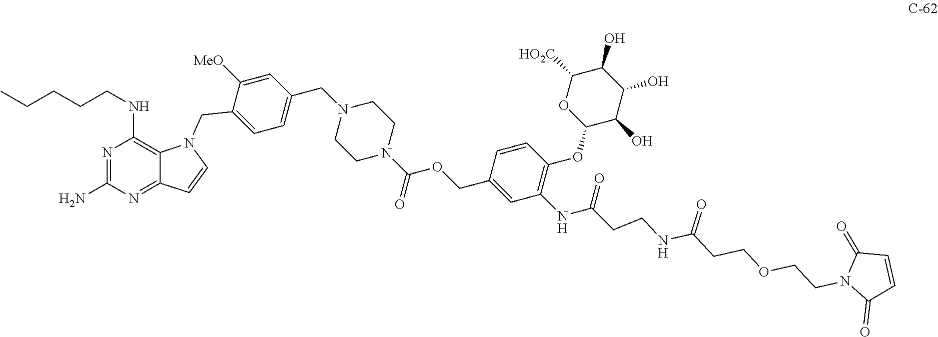 [SMILES: CCCCCNc1nc(N)nc2ccn(Cc3ccc(CN4CCN(C(=O)OCc5ccc(O[C@@H]6O[C@H](C(=O)O)[C@@H](O)[C@H](O)[C@H]6O)c(NC(=O)CCNC(=O)CCOCCN6C(=O)C=CC6=O)c5)CC4)cc3OC)c12]

(2S,3S,4S,5R,6S)-6-(4-(((4-(4-((2-amino-4-(pentylamino)-5H-pyrrolo[3,2-d]pyrimidin-5-yl)methyl)-3-methoxybenzyl)piperazine-1-carbonyl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic acid (C-62) was prepared as a solid as the TFA salt according to the scheme shown for Example (C-61), except 3-(2-(2,5-dioxo-2,5-dihydro-1H-pyrrol-1-yl)ethoxy)propanoic acid was used in place of 3-(2,5-dioxo-2,5-dihydro-1H-pyrrol-1-yl)propanoic acid in the last step: ¹H NMR (CD₃OD): δ 8.20 (d, 1H), 7.37 (d, 1H), 7.21 (m, 2H), 7.05 (m, 1H), 6.99 (d, 1H), 6.78 (m, 3H), 6.23 (d, 1H), 5.55 (s, 2H), 5.09 (s, 2H), 3.92 (m, 4H), 4.81 (d, 1H), 4.00 (s, 2H), 3.94 (s, 3H), 3.89 (d, 1H), 3.62 (m, 9H), 3.53 (m, 8H), 2.90 (m, 3H), 2.66 (t, 2H), 2.37 (t, 2H), 1.51 (m, 2H), 1.29 (m, 2H), 1.17 (m, 2H), 0.87 (t, 3H). LRMS [M+H]=1045.4.

### Example 63

### Synthesis of N-(2-((5-(4-(4-((2-amino-4-(pentylamino)-5H-pyrrolo[3,2-d]pyrimidin-5-yl)methyl)-3-methoxybenzyl)piperazin-1-yl)-2-methyl-5-oxopentan-2-yl)disulfanyl)ethyl)-3-(2,5-dioxo-2,5-dihydro-1H-pyrrol-1-yl)propanamide (C-63)

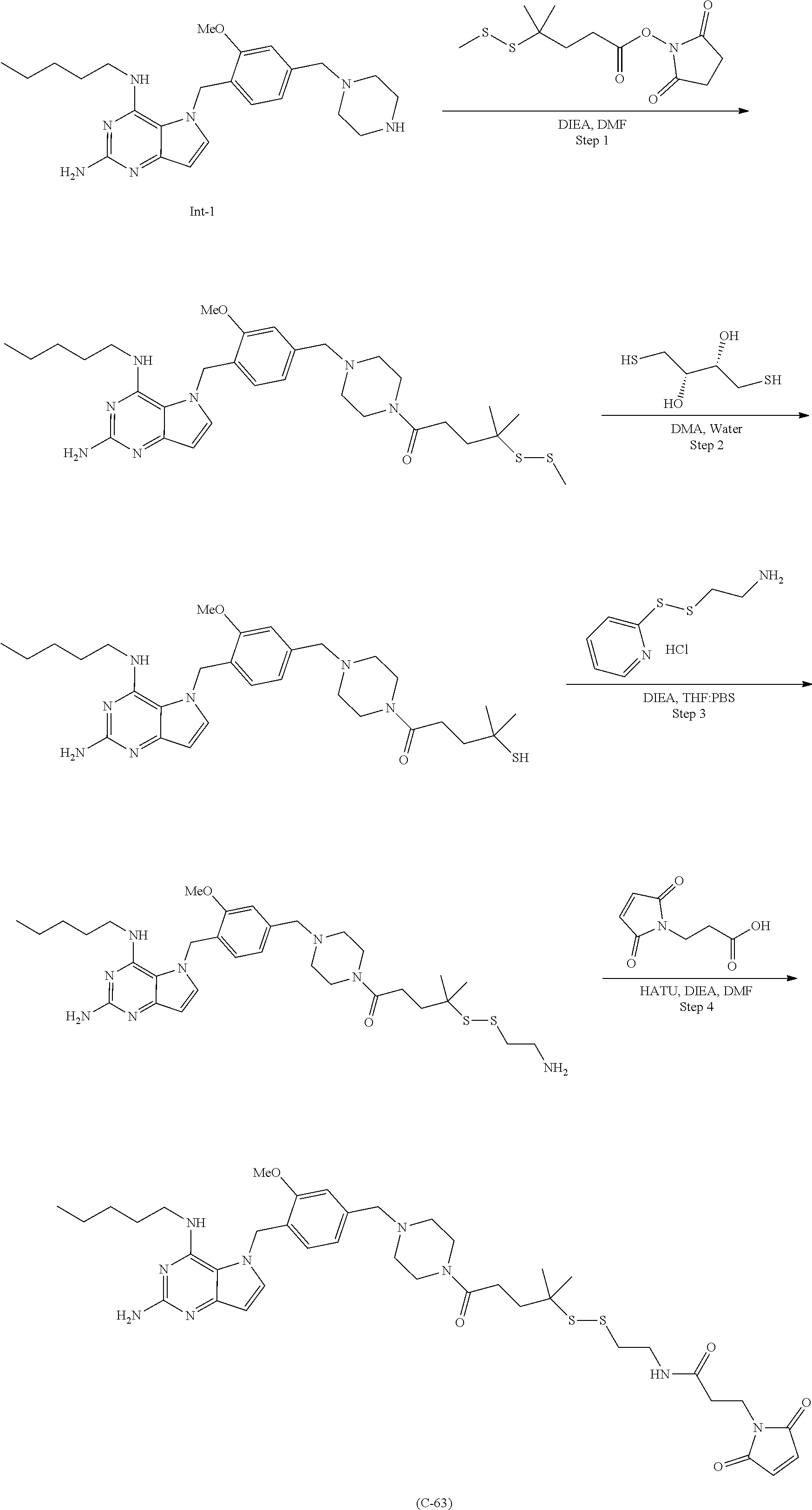

Step 1: A round bottom flask was charged with 5-(2-methoxy-5-(piperazin-1-ylmethyl)benzyl)-N4-pentyl-5H-pyrrolo[3,2-d]pyrimidine-2,4-diamine (Int-1, 1.0 equiv.), 2,5-dioxopyrrolidin-1-yl 4-methyl-4-(methyldisulfanyl)pentanoate (1.3 equiv.), Huenig's base (20.0 equiv.), and DMF (0.03 M). The reaction mixture was stirred at room temperature for 2 hours. The crude reaction mixture was then purified using RP-C18 ISCO (ACN:H₂O, with TFA as modifier) and then lyophilized to give 1-(4-(4-((2-amino-4-(pentylamino)-5H-pyrrolo[3,2-d]pyrimidin-5-yl)methyl)-3-methoxybenzyl)piperazin-1-yl)-4-methyl-4-(methyldisulfanyl)pentan-1-one as a solid as the TFA salt: LCMS [M+H]=614.3.

Step 2: A round bottom flask was charged with 1-(4-(4-((2-amino-4-(pentylamino)-5H-pyrrolo[3,2-d]pyrimidin-5-yl)methyl)-3-methoxybenzyl)piperazin-1-yl)-4-methyl-4-(methyldisulfanyl)pentan-1-one 1.0 equiv.), (2S,3S)-1,4-dimercaptobutane-2,3-diol (1.0 equiv.), and dimethyl acetamide:H₂O (1:1, 0.03 M). The reaction mixture was stirred at room temperature for 2 hours. The crude reaction mixture was then purified using RP-C18 ISCO (ACN:H₂O, with TFA as modifier) and then lyophilized to give 1-(4-(4-((2-amino-4-(pentylamino)-5H-pyrrolo[3,2-d]pyrimidin-5-yl)methyl)-3-methoxybenzyl)piperazin-1-yl)-4-mercapto-4-methylpentan-1-one as a solid as the TFA salt: LCMS [M+H]=568.3.

Step 3: A round bottom flask was charged with 1-(4-(4-((2-amino-4-(pentylamino)-5H-pyrrolo[3,2-d]pyrimidin-5-yl)methyl)-3-methoxybenzyl)piperazin-1-yl)-4-mercapto-4-methylpentan-1-one (1.0 equiv.), 2-(pyridin-2-yldisulfanyl)ethan-1-amine HCl salt (2.0 equiv.), Huenig's base (10.0 equiv.), and THF:PBS (1:1, 0.03 M). The reaction mixture was stirred at room temperature for 15 minutes. The crude reaction mixture was then purified using RP-C18 ISCO (ACN:H₂O, with TFA as modifier) and then lyophilized to give 1-(4-(4-((2-amino-4-(pentylamino)-5H-pyrrolo[3,2-d]pyrimidin-5-yl)methyl)-3-methoxybenzyl)piperazin-1-yl)-4-((2-aminoethyl)disulfanyl)-4-methylpentan-1-one as a solid as the TFA salt: LCMS [M+H]=643.4.

Step 4: A round bottom flask was charged with 1-(4-(4-((2-amino-4-(pentylamino)-5H-pyrrolo[3,2-d]pyrimidin-5-yl)methyl)-3-methoxybenzyl)piperazin-1-yl)-4-((2-aminoethyl)disulfanyl)-4-methylpentan-1-one (1.0 equiv.), 3-(2,5-dioxo-2,5-dihydro-1H-pyrrol-1-yl)propanoic Acid (1.0 equiv.), Huenig's base (5.0 equiv.), HATU (1.0 equiv.) and DMF (0.02 M).

The reaction mixture was stirred at room temperature for 2 hours. The crude reaction mixture was then purified using RP-C18 ISCO (ACN:H₂O, with TFA as modifier) and then lyophilized to give N-(2-((5-(4-(4-((2-amino-4-(pentylamino)-5H-pyrrolo[3,2-d]pyrimidin-5-yl)methyl)-3-methoxybenzyl)piperazin-1-yl)-2-methyl-5-oxopentan-2-yl)disulfanyl)ethyl)-3-(2,5-dioxo-2,5-dihydro-1H-pyrrol-1-yl)propanamide (C-63) as a solid as the TFA salt: ¹H NMR (CD₃OD): δ 7.37 (d, 1H), 7.26 (d, 1H), 7.08 (m, 1H), 6.83 (d, 1H), 6.81 (s, 2H), 6.24 (d, 1H), 5.58 (s, 2H), 4.37 (s, 2H), 4.20 (br, 4H), 3.97 (s, 3H), 3.75 (t, 2H), 3.55 (t, 2H), 3.38 (m, 2H), 3.38 (br, 4H), 2.72 (t, 2H), 2.55 (m, 2H), 2.45 (t, 2H), 1.89 (m, 2H), 1.54 (m, 2H), 1.31 (m, 8H), 1.19 (m, 2H), 0.88 (t, 3H). LRMS [M+H]=794.4.

### Example 64

### Synthesis of 1-(4-(4-((2-amino-4-(pentylamino)-5H-pyrrolo[3,2-d]pyrimidin-5-yl)methyl)-3-methoxybenzyl)piperazin-1-yl)-4-methyl-4-(methylthio)pentan-1-one (C-64)

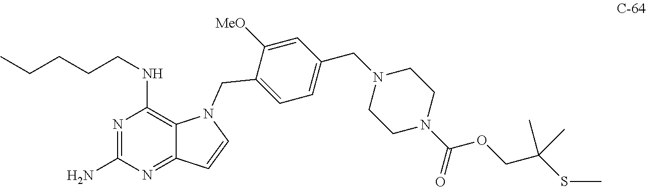 [SMILES: CCCCCNc1nc(N)nc2ccn(Cc3ccc(CN4CCN(C(=O)OCC(C)(C)SC)CC4)cc3OC)c12]

1-(4-(4-((2-amino-4-(pentylamino)-5H-pyrrolo[3,2-d]pyrimidin-5-yl)methyl)-3-methoxybenzyl)piperazin-1-yl)-4-methyl-4-(methylthio)pentan-1-one (C-63) was prepared following the procedure described for intermediate Int-1, except using 4-methyl-4-(methylthio)-1-(piperazin-1-yl)pentan-1-one in place of tert-butyl piperazine-1-carboxylate in step 3. The crude reaction mixture was purified using RP-C18 ISCO (ACN:H₂O, with TFA as modifier) and then lyophilized to give 1-(4-(4-((2-amino-4-(pentylamino)-5H-pyrrolo[3,2-d]pyrimidin-5-yl)methyl)-3-methoxybenzyl)piperazin-1-yl)-4-methyl-4-(methylthio)pentan-1-one (C-64) as a solid as the TFA salt: ¹H NMR (CD₃OD): δ 7.36 (d, 1H), 7.25 (d, 1H), 7.05 (m, 1H), 6.81 (d, 1H), 6.24 (d, 1H), 5.58 (s, 2H), 4.34 (s, 2H), 3.90 (br, 4H), 3.96 (s, 3H), 3.55 (t, 2H), 3.28 (br, 4H), 2.55 (m, 2H), 1.95 (s, 3H), 1.80 (m, 2H), 1.54 (m, 2H), 1.31 (m, 2H), 1.27 (s, 6H), 1.19 (m, 2H), 0.88 (t, 3H). LRMS [M+H]=582.4.

### Example 65

### Synthesis of (2S,3S,4S,5R,6S)-6-(4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic Acid (C-65)

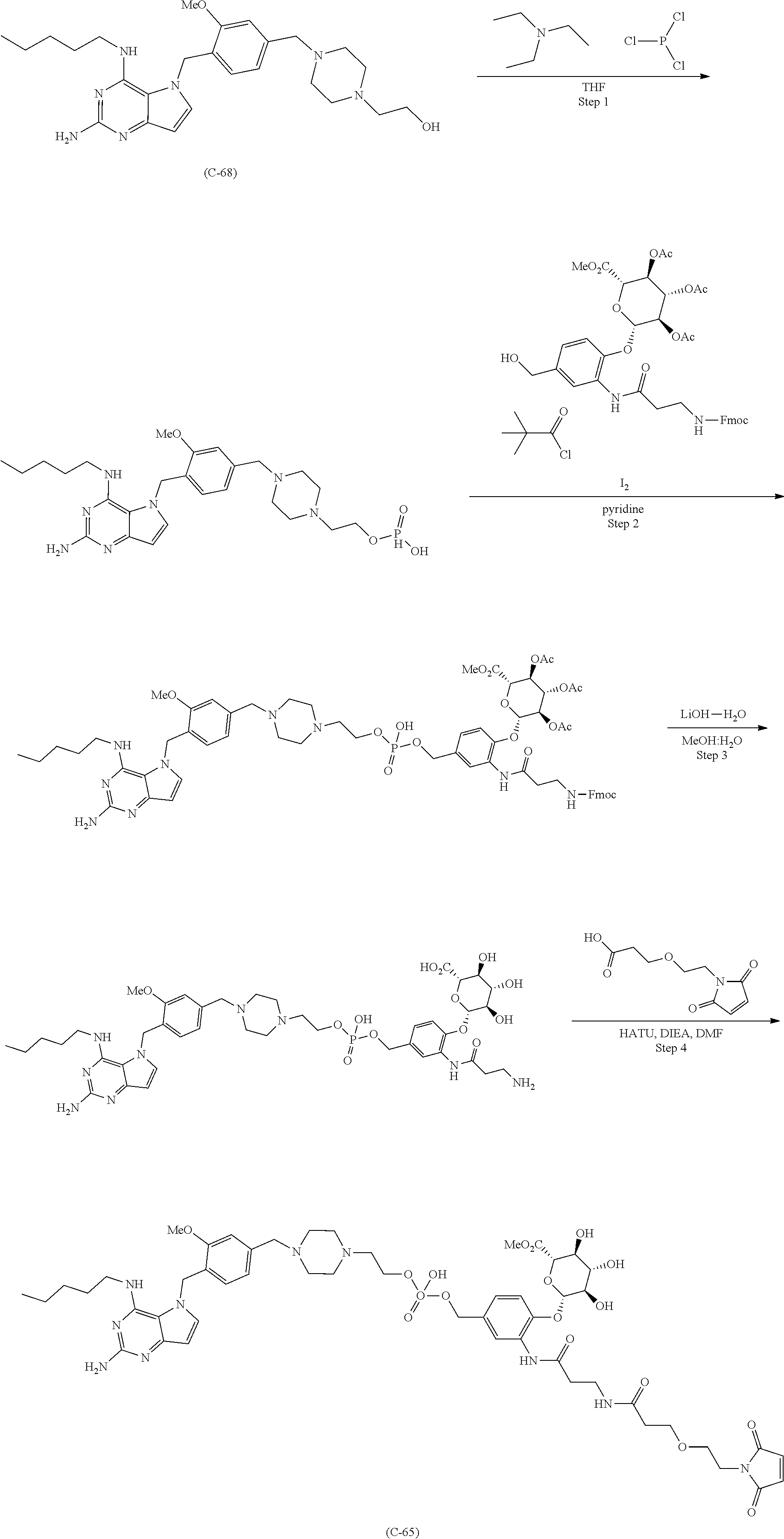

Step 1: A round bottom flask was charged with 2-(4-(4-((2-amino-4-(pentylamino)-5H-pyrrolo[3,2-d]pyrimidin-5-yl)methyl)-3-methoxybenzyl)piperazin-1-yl)ethan-1-ol (C-68) (1.0 equiv.), trichlorophosphane (3.0 equiv.), triethylamine (9.0 equiv.), and THF (0.2 M) at 0° C. and allowed to stir for 1 h. The reaction was then quenched by the slow addition of ice-water and washed with EtOAc 3×. The aqueous layer containing the desired product was then lyophilized. 2-(4-(4-((2-amino-4-(pentylamino)-5H-pyrrolo[3,2-d]pyrimidin-5-yl)methyl)-3-methoxybenzyl)piperazin-1-yl)ethyl hydrogen phosphonate was isolated and used in the next step without further purification: LCMS [M+H]=546.3.

Step 2: A round bottom flask was charged with (2S,3R,4S,5S,6S)-2-(2-(3-((((9H-fluoren-9-yl)methoxy)carbonyl)amino)propanamido)-4-(hydroxymethyl)phenoxy)-6-(methoxycarbonyl)tetrahydro-2H-pyran-3,4,5-triyl triacetate (1.0 equiv.), 2-(4-(4-((2-amino-4-(pentylamino)-5H-pyrrolo[3,2-d]pyrimidin-5-yl)methyl)-3-methoxybenzyl)piperazin-1-yl)ethyl hydrogen phosphonate (2.0 equiv.), pivaloyl chloride (42.0 equiv.), and pyridine (0.03 M). The reaction mixture was stirred at room temperature for 2 hours. At this point diiodide (1.06 equiv.) in pyridine:H₂O (1:0.1, 0.14 M) was added and the mixture stirred for 10 min. The crude reaction mixture was then purified using RP-HPLC (0.035% TFA in ACN:0.05% TFA in H₂O, C18 column) to obtain (2S,3R,4S,5S,6S)-2-(2-(3-((((9H-fluoren-9-yl)methoxy)carbonyl)amino)propanamido)-4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)phenoxy)-6-(methoxycarbonyl)tetrahydro-2H-pyran-3,4,5-triyl triacetate as a solid as the TFA salt: LCMS [M+H]=1292.5.

Step 3: A round bottom flask was charged with (2S,3R,4S,5S,6S)-2-(2-(3-((((9H-fluoren-9-yl)methoxy)carbonyl)amino)propanamido)-4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)phenoxy)-6-(methoxycarbonyl)tetrahydro-2H-pyran-3,4,5-triyl triacetate (1.0 equiv.), lithium hydroxide-H₂O (10.0 equiv.) and MeOH:H₂O (3:1.5, 0.007 M). The reaction mixture was stirred at room temperature for 2 hours. The crude reaction mixture was then purified using RP-C18 ISCO (ACN:H₂O, with TFA as modifier) and then lyophilized to give (2S,3S,4S,5R,6S)-6-(4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)-2-(3-aminopropanamido)phenoxy)-3,4,5-trihydroxytetrahydro-2H-pyran-2-carboxylic acid as a solid as the TFA salt: LCMS [M+H]=930.4.

Step 4: A round bottom flask was charged with (2S,3S,4S,5R,6S)-6-(4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)-2-(3-aminopropanamido)phenoxy)-3,4,5-trihydroxytetrahydro-2H-pyran-2-carboxylic acid (1.0 equiv.), 3-(2-(2,5-dioxo-2,5-dihydro-1H-pyrrol-1-yl)ethoxy)propanoic acid (1.0 equiv.), Huenig's base (6.0 equiv.), HATU (1.0 equiv.) and DMF (0.005 M). The reaction was kept stirring at room temperature for 15 minutes. The crude reaction mixture was then purified by RP-HPLC (0.035% TFA in ACN:0.05% TFA in H₂O, C18 column) to afford (2S,3S,4S,5R,6S)-6-(4-((((2-(4-(4-((2-amino-4-(pentylamino)-5H-pyrrolo[3,2-d]pyrimidin-5-yl)methyl)-3-methoxybenzyl)piperazin-1-yl)ethoxy)(hydroxy)phosphoryl)oxy)methyl)-2-(3-(3-(2-(2,5-dioxo-2,5-dihydro-1H-pyrrol-1-yl)ethoxy)propanamido)propanamido)phenoxy)-3,4,5-trihydroxytetrahydro-2H-pyran-2-carboxylic acid (C-65) as a solid as the TFA salt: ¹H NMR (CD₃OD): δ 8.19 (s, 1H), 7.37 (d, 1H), 7.14 (m, 3H), 6.79 (s, 2H), 6.77 (d, 1H), 6.22 (d, 1H), 5.53 (s, 2H), 4.86 (s, 2H), 4.84 (d, 1H), 4.08 (s, 2H), 3.95 (d, 1H), 3.92 (s, 3H), 4.00 (br, 4H), 3.76 (s, 2H), 3.62 (m, 5H), 3.53 (m, 10H), 3.27 (m, 2H), 2.85 (m, 4H), 2.63 (m, 2H), 2.37 (t, 2H), 1.52 (m, 2H), 1.31 (m, 2H), 1.17 (m, 2H), 0.88 (t, 3H). LRMS [M+H/2Z]=563.4.

### Example 66

### Synthesis of (2R,2′R)-3,3′-((2-((2-(4-(4-((2-amino-4-(pentylamino)-5H-pyrrolo[3,2-d]pyrimidin-5-yl)methyl)-3-methoxybenzyl)piperazin-1-yl)-2-oxoethoxy)imino)propane-1,3-diyl)bis(sulfanediyl))bis(2-aminopropanoic acid) (C-66)

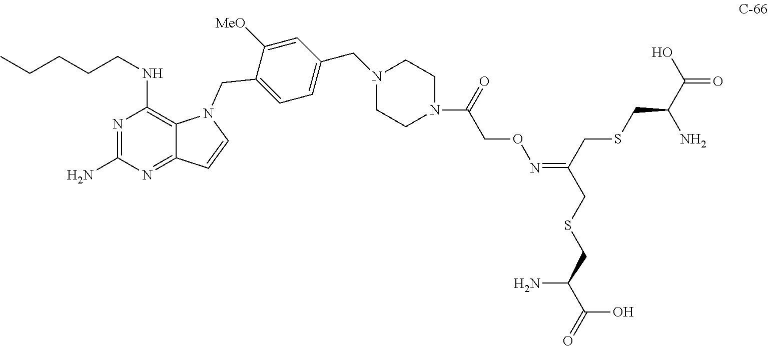 [SMILES: CCCCCNc1nc(N)nc2ccn(Cc3ccc(CN4CCN(C(=O)CON=C(CSC[C@H](N)C(=O)O)CSC[C@H](N)C(=O)O)CC4)cc3OC)c12]

A round bottom flask was charged with 1-(4-(4-((2-amino-4-(pentylamino)-5H-pyrrolo[3,2-d]pyrimidin-5-yl)methyl)-3-methoxybenzyl)piperazin-1-yl)-2-(aminooxy)ethan-1-one (C-35) (2.4 equiv.), (2R,2′R)-3,3′-((2-oxopropane-1,3-diyl)bis(sulfanediyl))bis(2-aminopropanoic acid) (1.0 equiv.), and ethanol (0.02 M). The reaction mixture was stirred at room temperature for 30 min. The crude reaction mixture was purified using RP-C18 ISCO (ACN:H₂O, with TFA as modifier) and then lyophilized to give (2R,2′R)-3,3′-((2-((2-(4-(4-((2-amino-4-(pentylamino)-5H-pyrrolo[3,2-d]pyrimidin-5-yl)methyl)-3-methoxybenzyl)piperazin-1-yl)-2-oxoethoxy)imino)propane-1,3-diyl)bis(sulfanediyl))bis(2-aminopropanoic acid) (C-66) as a solid: ¹H NMR (CD₃OD): δ 7.35 (d, 1H), 7.28 (d, 1H), 7.05 (m, 1H), 6.80 (d, 1H), 6.23 (d, 1H), 5.57 (s, 2H), 4.32 (s, 2H), 4.20 (m, 1H), 4.05 (m, 1H), 3.94 (s, 3H), 3.81 (m, 4H), 3.55 (m, 2H), 3.44 (m, 2H), 3.20 (m, 4H), 2.96 (m, 1H), 2.88 (m, 1H), 1.53 (m, 2H), 1.31 (m, 2H), 1.18 (m, 2H), 0.88 (t, 3H). LRMS [M+H]=789.3.

### Example 67

### Synthesis of (R)-2-amino-6-((((R)-2-amino-2-carboxyethyl)thio)methyl)-17-(4-(4-((2-amino-4-(pentylamino)-5H-pyrrolo[3,2-d]pyrimidin-5-yl)methyl)-3-methoxybenzyl)piperazin-1-yl)-10,17-dioxo-8,14-dioxa-4-thia-7,11-diazaheptadec-6-enoic Acid (C-67)

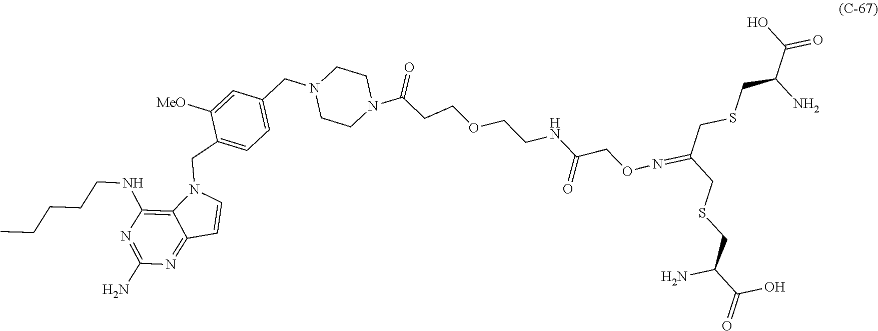 [SMILES: CCCCCNc1nc(N)nc2ccn(Cc3ccc(CN4CCN(C(=O)CCOCCNC(=O)CON=C(CSC[C@H](N)C(=O)O)CSC[C@H](N)C(=O)O)CC4)cc3OC)c12]

A round bottom flask was charged with N-(2-(3-(4-(4-((2-amino-4-(pentylamino-5H-pyrrolo[3,2-d]pyrimidin-5-yl)methyl)-3-methoxybenzyl)piperazin-1-yl)-3-oxopropoxy)ethyl)-2-(aminooxy)acetamide (C-37) (2.4 equiv.), (2R,2′R)-3,3′-((2-oxopropane-1,3-diyl)bis(sulfanediyl))bis(2-aminopropanoic acid) (1.0 equiv.), and ethanol (0.02 M). The reaction mixture was stirred at room temperature for 30 min. The crude reaction mixture was purified using RP-C18 ISCO (ACN:H₂O, with TFA as modifier) and then lyophilized to give (R)-2-amino-6-((((R)-2-amino-2-carboxyethyl)thio)methyl)-17-(4-(4-((2-amino-4-(pentylamino)-5H-pyrrolo[3,2-d]pyrimidin-5-yl)methyl)-3-methoxybenzyl)piperazin-1-yl)-10,17-dioxo-8,14-dioxa-4-thia-7,11-diazaheptadec-6-enoic acid (C-67) as a solid: ¹H NMR (CD₃OD): δ 7.36 (d, 1H), 7.29 (d, 1H), 7.07 (m, 1H), 6.80 (d, 1H), 6.24 (d, 1H), 5.57 (s, 2H), 4.57 (s, 2H), 4.31 (m, 2H), 4.11 (m, 1H), 4.03 (m, 1H), 3.95 (s, 3H), 3.86 (br, 4H), 3.73 (t, 2H), 3.54 (m, 6H), 3.40 (m, 2H), 3.20 (m, 8H), 2.96 (m, 2H), 2.67 (t, 2H), 1.52 (m, 2H), 1.30 (m, 2H), 1.19 (m, 2H), 0.88 (t, 3H). LRMS [M+H]=904.4.

### Example 68

### Synthesis of 2-(4-(4-((2-amino-4-(pentylamino)-5H-pyrrolo[3,2-d]pyrimidin-5-yl)methyl)-3-methoxybenzyl)piperazin-1-yl)ethan-1-ol (C-68)

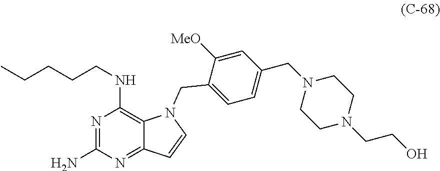 [SMILES: CCCCCNc1nc(N)nc2ccn(Cc3ccc(CN4CCN(CCO)CC4)cc3OC)c12]

A round bottom flask was charged with 5-(2-methoxy-4-(piperazin-1-ylmethyl)benzyl)-N4-pentyl-5H-pyrrolo[3,2-d]pyrimidine-2,4-diamine (Int-1, 1.0 equiv.), 2-bromoethan-1-ol (1.3 equiv.), triethylamine (20.0 equiv.), and acetonitrile (0.03 M). The reaction mixture was stirred at room temperature for 2 hours. The crude reaction mixture was then purified by ISCO chromatography (0-10% MeOH:DCM, gradient) to afford 2-(4-(4-((2-amino-4-(pentylamino)-5H-pyrrolo[3,2-d]pyrimidin-5-yl)methyl)-3-methoxybenzyl)piperazin-1-yl)ethan-1-ol (C-68) as a solid: ¹H NMR (CD₃OD): δ 7.22 (d, 1H), 7.08 (d, 1H), 6.83 (d, 1H), 6.55 (d, 1H), 6.10 (d, 1H), 5.39 (s, 2H), 3.93 (s, 3H), 3.66 (t, 2H), 3.50 (s, 2H), 3.32 (m, 2H), 3.20 (s, 1H), 2.51 (m, 10H), 1.37 (m, 2H), 1.27 (m, 2H), 1.25 (s, 1H), 1.12 (m, 2H), 0.86 (t, 3H). LRMS [M+H]=482.4.

### Example 69

Compounds of Formula (I) were assayed to measure their activity as toll-like receptor 7 agonists.

Reporter Gene Assay

Human embryonic kidney 293 (HEK293) cells were stably transfected with human TLR7 and an NF-kB-driven luciferase reporter vector (pNifty-Luciferase). As a control assay, normal HEK293 transfected with pNifty-Luc were used. Cells were cultured in DMEM supplemented with 2 mM L-glutamine, 10% heart inactivated FBS, 1% penicillin and streptomycin, 2 μg/ml puromycin (InvivoGen #ant-pr-5) and 5 μg/ml of blasticidin (Invitrogen #46-1120). Bright-Glo™ Luciferase assay buffer and substrate were supplied by Promega #E263B and #E264B (assay substrate and buffer respectively). 384 well clear-bottom plates were supplied by Greiner bio-one (#789163-G) and were custom bar-coded plates.

Cells were plated at 25,000 cells/well in 384-well plates in a final volume of 50 μl of media. Cells were allowed to adhere to the plates after overnight (18 hours) culture at 37° C. and 5% CO₂. Serially diluted experimental and positive control compounds were then dispensed to each well and incubated for 7 hours at 37° C. and 5% CO₂. Cells stimulated with DMSO alone also serve as negative controls. After the incubation, 30 μl of the pre-mix assay buffer and substrate buffer were added to each well according to manufacturer's instructions. The luminescence signal was read on a CLIPR machine with an integration time of 20 seconds per plate.

Dose response curves are generated for each compound and EC₅₀ values were determined as the concentration that gives 50% of the maximal signal.

Selected Assay Results

Various compounds of Formula (I), in free form or in pharmaceutically acceptable salt form, exhibit pharmacological properties, for example, as indicated by the in vitro tests described in this application. The EC₅₀ value in those experiments is given as that concentration of the test compound in question that provokes a response halfway between the baseline and maximum responses. In other examples, compounds of Formula (I) have EC₅₀ values in the range from 1 nM to 2 μM. In other examples, compounds of Formula (I) have EC₅₀ values in the range from 1 nM to 1 μM. In other examples, compounds of Formula (I) have EC₅₀ values in the range from 1 nM to 500 nM. In other examples, compounds of Formula (I) have EC₅₀ values in the range from 1 nM to 250 nM. In other examples, compounds of Formula (I) have EC₅₀ values in the range from 1 nM to 100 nM. In other examples, compounds of Formula (I) have EC₅₀ values in the range from 1 nM to 50 nM. In other examples, compounds of Formula (I) have EC₅₀ values in the range from 1 nM to 25 nM. In other examples, compounds of Formula (I) have EC₅₀ values in the range from 1 nM to 10 nM.

To illustrate the in-vitro activity of the compounds of the invention, the EC₅₀ values for TLR7 stimulation by certain compounds of Formula (I) are listed in Table 14. Cysteine adduct are thought to be putative catabolytes that arise from degradation within the lysosome (Bioconjugate Chem. 2006, 17, 114-124). Certain compounds of Table 14 are the result of derivatization of the corresponding parent compound with cysteine.

TABLE 14
EC50 of TLR7 Agonist Compounds

| | Human TLR7 |
|---|---|
| Compound | EC₅₀ (nM) |
| Number | HEK293 |
| C-2 | 10 |
| C-3 | 96 |
| C-4 | 35 |
| C-6 | 16 |
| C-7 | 77 |
| C-8 | 32 |
| C-10 | 157 |
| C-12 | 144 |
| C-14 | 8 |
| C-16 | 289 |
| C-18 | 518 |
| C-19 | 2 |
| C-20 | 11 |
| C-22 | 598 |
| C-24 | 277 |
| C-26 | 134 |
| C-28 | 230 |
| C-34 | 585 |
| C-35 | 5 |
| C-36 | 4 |
| C-37 | 57 |
| C-38 | 278 |
| C-39 | 192 |
| C-40 | 2101 |
| C-41 | 52 |
| C-42 | 1 |
| C-43 | 6 |
| C-44 | 2 |
| C-45 | 11 |
| C-48 | 1900 |
| C-49 | 264 |
| C-51 | 80 |
| C-53 | 753 |
| C-57 | 16 |
| C-64 | 3 |
| C-66 | 2 |
| C-67 | 30 |
| C-68 | <1 |

### Example 70

### Synthesis of Constructs (C-70), (C-71), (C-72) and (C-73)

| Construct No. | Sequence and chemical composition | |
|---|---|---|
| C-70 | 5′OH-GGACGUACGC(UUCG)GCGUACGUCC-3′ | SEQ ID NO: 336 |
| C-71 | 5′ppp-GGACGUACGC(UUCG)GCGUACGUCC-3′ | SEQ ID NO: 334 |
| C-72 | 5′OH-GGACGUACGC(UXCG)GCGUACGUCC-3′ | SEQ ID NO: 337 |
| C-73 | 5′ppp-GGACGUACGC(UXCG)GCGUACGUCC-3′ | SEQ ID NO: 335 |

RNA synthesis of constructs C-70 and C-72 was performed on an Applied Biosystems Expedite™ 8900 Nucleic Acid Synthesis System according to procedures outlined in the User's Guide. 5′-DMT (4,4′-DiMethoxTrityl)-2′-TOM (TriisopropylsilylOxyMethyl) phosphoramidites were obtained from ChemGenes (Wilmington, Mass.) and, as per the supplier's recommendation, a coupling time is of 6 minutes was used. The 5′-OH-oligoribonucleotides were synthesized on 1 μmol scale. The RNA phosphoramidite monomers used for the synthesis were as follows; (A): 5′-Dimethoxytrityl-N-acetyl-Adenosine,2′-O-triisopropylsilyloxymethyl-3′-[(2-cyanoethyl)-(N,N-diisopropyl)]-phosphoramidite, (C): 5′-Dimethoxytrityl-N-acetyl-Cytidine,2′-O-triisopropylsilyloxymethyl-3′-[(2-cyanoethyl)-(N,N-diisopropyl)]-phosphoramidite, (G): 5′-Dimethoxytrityl-N-acetyl-Guanosine,2′-O-triisopropylsilyloxymethyl-3′-[(2-cyanoethyl)-(N,N-diisopropyl)]-phosphoramidite, (U): 5′-Dimethoxytrityl-Uridine,2′-O-triisopropylsilyloxymethyl-3′-[(2-cyanoethyl)-(N,N-diisopropyl)]-phosphoramidite. Constructs C-72 and C-73 contain a C8-Alkyne-dT base which is designated as (X): C8-Alkyne-dT was installed utilizing C8-Alkyne-dT β-Cyanoethyl Phosphoramidite purchased from Glen Research (Sterling, Va., Catalog Number:10-1540-xx). 1 μm CPG (Controlled Pore Glass) solid supports for 2′-TOM RNA from Glen Research. The reagents used in the Expedite 8909 protocol were from Glen Research and included: Acetonitrile, Activator (0.25 M 5-Ethylthio-1H-Tetrazole in Anhydrous Acetonitrile), Cap A (Tetrahydrofuran/Acetic Anhydride), Cap B (10% 1-Methylimidazole in Tetrahydrofuran/Pyridine), Oxidizer (0.02 M Iodine in Tetrahydrofuran/Water/Pyridine), and Deblock (3% dichloroacetic acid DCM). RNA deprotection and desalting was performed according to procedures outlined in Glen-Pak™ DNA and RNA Purification User's Guide. Desalting was performed on GE Healthcare Illustra™ NAP™ Columns, NAP-25.

Analytical Methods: Ion Pairing-Reverse Phase (IP-RP) LC-MS analysis was performed using Waters Acquity UPLC coupled to an ion trap mass spectrometer (SQ Detector 2, Waters) equipped with an electrospray source operating in negative ionization mode. All samples were chromatographed on a Waters Acquity UPLC BEHC18 column (2.1×50 mm; 1.7 um particle size) at 60° C. column temperature. Separation of the analytes was achieved using a gradient of 100 mM HexaFluorolsoPropanol (HFIP) and 10 mM TEA as eluent A and methanol as eluent B with a flow rate of 0.8 mL/min.

RP-HPLC purification: The samples were applied to a Waters Xbridge Prep C18 column (19×50 mm, 5 um) and separated using a linear gradient from 20 to 50% buffer B in buffer A for 10 min at a flow rate of 30 mL/min. Buffer A was 100 mM Triethylamine bicarbonate buffer (TEAB; for preparation of buffer 1.72 liters of water was mixed with 284 mL triethylamine, then bubbled with CO₂ (dry ice for a few hours until pH reach 8.6)) and buffer B was 100 mM TEAB in 50% acetonitrile. The product fractions were collected, evaporated and desalted by co-evaporation in milli-Q water (Millipore).

### Construct C-70: 5′OH-GGACGUACGCUUCGGCGUACGUCC (SEQ ID NO: 936).

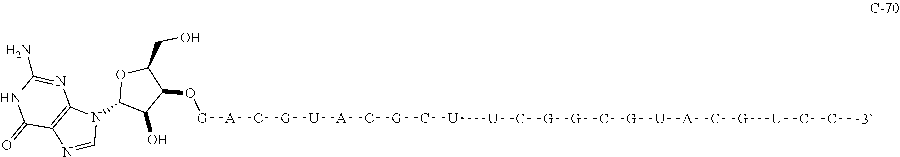

After cleavage from the support, C-70 (SEQ ID NO: 936) was purified by GlenPak RNA cartridges following procedures outlined in the user's guide. The collected elutions were dried in spinning vacuum and characterized by mass spectrometry on a Waters UPLC: found [M+H]=7661.0

### Construct C-72: 5′OH-GGACGUACGCUXCGGCGUACGUCC (SEQ ID NO: 937)

Purification was performed in similar fashion to that used for construct C-70. Characterization by mass spectrometry on a Waters UPLC: found [M+H]=7748.0

Construct C-71: 5′ ppp-GGACGUACGC (UUCG) GCGUACGUCC-3′ (SEQ ID NO: 334)

Using the procedure published in Angew Chem Int Ed, 2014, 53, 4694-4698, the 5′-triphosphate (ppp) was installed onto C-70 to give construct C-71.

### Step 1: Synthesis of 5′-Decyl-NH-Triphosphate-GGACGUACGCUUCGGCGUACGUCC. (SEQ ID NO: 936)

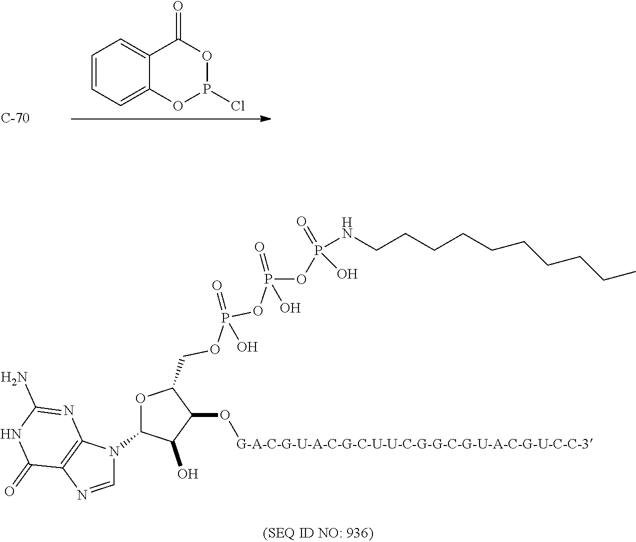

### a) Synthesis of 5′-Cyclotriphosphate Oligoribonucleotides.

The support-bound, fully protected 5-OH-oligoribonucleotide C-70 (0.2-1.0 μmol) was dried for 3 h under vacuum within a synthesis column and subsequently washed with anhydrous dioxane/pyridine solution (3:1 v/v, 2×2 mL). Under argon, a freshly prepared 1 M solution of 2-chloro-4H-1,3,2-benzodioxaphosphorin-4-one in dry dioxane (100 μL, 100 μmol) was injected into a vial containing 2 mL of anhydrous pyridine/dioxane (1:3 v/v) to give a 50 mM solution of the phosphitylating reagent. The reaction was started by drawing the 2-chloro-4H-1,3,2-benzodioxaphosphorin-4-one solution from the vial through the synthesis column using a gas-tight syringe and repeatedly drawing in and expelling from the synthesis column during a reaction time of 30 min. Next, 1 mL of 0.5 M bis(tri-n-butylammonium) pyrophosphate in dry DMF and 238 μL (1 mmol) of dry tri-n-butylamine were mixed to give a 0.5 M tetra(tri-n-butylammonium) pyrophosphate solution. The solution was quickly pushed through the column, thereby replacing the dioxane/pyridine solution containing the excess phosphitylating agent. After 10 min the column was washed with anhydrous acetonitrile (3 mL). A 5.5 M tert-butyl hydroperoxide solution in decane (300 μL) was dissolved in 2 mL of anhydrous acetonitrile to give an approximately 0.7 M homogeneous solution. The synthesis support was brought into contact with this solution for 15 min and then washed with anhydrous acetonitrile (6 mL).

### b) Decylamine Tagging of 5′-Cyclotriphosphate Oligoribonucleotides.

The support in the column was brought into contact with a solution of dry n-decylamine (300 μL, 1.5 mmol) in dry acetonitrile (1 mL) and the solution was repeatedly pushed through the column for 3 min by means of gas-tight syringes. Following a further wash step with anhydrous acetonitrile (9 mL) the synthesis support was dried in a stream of argon.

### c) Cleavage and Deprotection of 5′-Decyl-NH-Triphosphate Oligoribonucleotides.

The 5′-decyl-NH-triphosphate oligoribonucleotide was brought into contact with a freshly prepared solution of 40% aqueous methylamine and concentrated aqueous ammonia (AMA, 2 mL, 1:1 v/v) using two syringes. After 30 min cleavage time the solution was transferred to a new vial and the support was rinsed with AMA (1 mL). The combined solution and washing was heated for 10 min at 65° C. After cooling on ice the solution was evaporated to dryness on a lyophilizer. Subsequently, 1 mL of 1 M tetra-n-butylammonium fluoride (TBAF) in THE was added, the mixture was heated for 15 minutes at 65° C. water bath to dissolve powder material. These solutions were placed at room temperature for 16 h. The reaction was quenched with 1 mL sterile aqueous 1 M TEAB and desalted on a NAP-25 column using sterile water as eluent.

### d) Purification of 5′-Decyl-NH-Triphosphate-GGACGUACGCUUCGGCGUACGUCC (SEQ ID NO: 936).

Purified by RP-HPLC (10 min method, mobile phase A 0.1 M TEAB buffer, mobile phase B:1/1 mixture of acetonitrile/0.1 M TEAB), collected fractions were dried in spinning vacuum to afford the product. This material was then used in the next reaction.

### Step 2: Synthesis of 5′Triphosphate-GGACGUACGCUUCGGCGUACGUCC (C-71) (SEQ ID NO: 936).

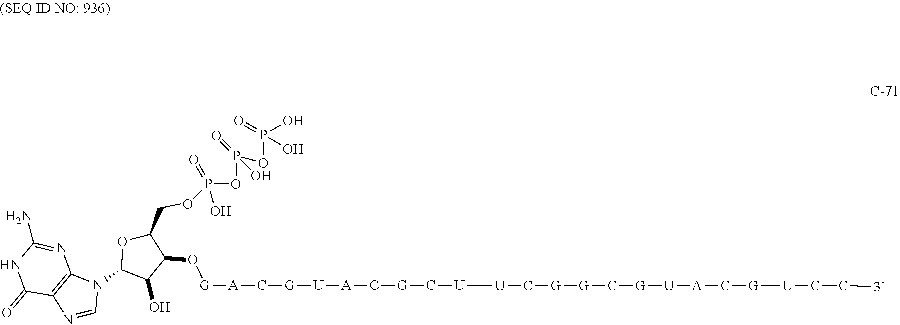

The removal of the decylamine tag (100 nmol 5′-decyl-NH-triphosphate-GGACGUACGCUUCGGCGUACGUCC (SEQ ID NO: 936)) was carried out using two vials, each was dissolved in 400 μL pH 3.8 deprotection buffer (100 mM sodium acetate, pH 3.8) and heated at 65° C. for 70 min. Subsequently, the reaction mixture was cooled on ice and desalted by NAP column (2 NAP-25 columns in total). NAP column was pre-equilibrated with 25 mL of water, the 400 μL of sample was diluted to 1.5 mL with water, loaded to NAP column, then 1 mL of water was used for column equilibration. It was then eluted with 2.5 mL of water. Eluents were combined and lyophilized to afford the product. Obtained product was dissolved in 2 mL of molecular biology grade water (nuclease free), 2 μL of this solution was diluted with 27 μL of molecular grade water (nuclease free). This solution was used for NanoDrop™ nucleic acid quantification at 260 nM UV absorbance and based on this measurement we obtained 1.79 mg of material. Characterization by mass spec on a Waters UPLC: found [M+H]=7902.0

Construct C-73: 5′ ppp-GGACGUACGC (UXCG) GCGUACGUCC-3′ (SEQ ID NO: 335) Construct C-73 was obtained by the addition of the 5-triphosphate (ppp) onto C-72 using the same procedure described for C-71. Characterization by mass spec on a Waters UPLC: found [M+H]=7989.0

### Example 71

Constructs (C-70), (C-71), (C-72) and (C-73) were assayed to measure their activity as RIG-I agonists using the assay described in Example 79. The EC₅₀ values are given in the following Table:

| Construct No. | EC₅₀ (μM) |
|---|---|
| C70 | 0.17 |
| C71 | 0.76 |
| C72 | 1.54 |
| C73 | 1.29 |

### Example 72: Generation of Anti-DC-SIGN Antibodies

### Generation of Expression Constructs for Human and Cynomolgus Monkey DC-SIGN

Full length human DC-SIGN DNA (SEQ ID NO: 306) was synthesized based on amino acid sequences from the Uniprot databases (Q9NNX6, SEQ ID NO: 303), the cyno DC-SIGN DNA (SEQ ID NO: 312) was synthesized based on cyno DC-SIGN amino acid sequence (SEQ ID NO: 311). All synthesized DNA fragments were cloned into appropriate expression vectors.

TABLE 15
Amino Acid and Nucleotide Sequence Information for DC-SIGN proteins

| | | |
|---|---|---|
| Human DC-SIGN | | |
| Full length AA | MSDS KEPRLQQLGL LEEEQLRGLG | SEQ ID |
| | FRQTRGYKSL AGCLGHGPLV LQLLSFTLLA GLLVQVSKVP SSISQEQSRQ | NO: 303 |
| | DAIYQNLTQL KAAVGELSEK SKLQEIYQEL TQLKAAVGEL PEKSKLQEIY | |
| | QELTRLKAAV GELPEKSKLQ EIYQELTWLK AAVGELPEKS KMQEIYQELT | |
| | RLKAAVGELP EKSKQQEIYQ ELTRLKAAVG ELPEKSKQQE IYQELTRLKA | |
| | AVGELPEKSK QQEIYQELTQ LKAAVERLCH PCPWEWTFFQ GNCYFMSNSQ | |
| | RNWHDSITAC KEVGAQLVVI KSAEEQNFLQ LQSSRSNRFT WMGLSDLNQE | |
| | GTWQWVDGSP LLPSFKQYWN RGEPNNVGEE DCAEFSGNGW NDDKCNLAKF | |
| | WICKKSAASC SRDEEQFLSP APATPNPPPA | |
| Full length | AT GAGTGACTCC AAGGAACCAA | SEQ ID |
| DNA | GACTGCAGCA GCTGGGCCTC CTGGAGGAGG AACAGCTGAG AGGCCTTGGA | NO: 306 |
| | TTCCGACAGA CTCGAGGATA CAAGAGCTTA GCAGGGTGTC TTGGCCATGG | |
| | TCCCCTGGTG CTGCAACTCC TCTCCTTCAC GCTCTTGGCT GGGCTCCTTG | |
| | TCCAAGTGTC CAAGGTCCCC AGCTCCATAA GTCAGGAACA ATCCAGGCAA | |
| | GACGCGATCT ACCAGAACCT GACCCAGCTT AAAGCTGCAG TGGGTGAGCT | |
| | CTCAGAGAAA TCCAAGCTGC AGGAGATCTA CCAGGAGCTG ACCCAGCTGA | |
| | AGGCTGCAGT GGGTGAGCTT CCAGAGAAAT CTAAGCTGCA GGAGATCTAC | |
| | CAGGAGCTGA CCCGGCTGAA GGCTGCAGTG GGTGAGCTTC CAGAGAAATC | |
| | TAAGCTGCAG GAGATCTACC AGGAGCTGAC CTGGCTGAAG GCTGCAGTGG | |
| | GTGAGCTTCC AGAGAAATCT AAGATGCAGG AGATCTACCA GGAGCTGACT | |
| | CGGCTGAAGG CTGCAGTGGG TGAGCTTCCA GAGAAATCTA AGCAGCAGGA | |
| | GATCTACCAG GAGCTGACCC GGCTGAAGGC TGCAGTGGGT GAGCTTCCAG | |
| | AGAAATCTAA GCAGCAGGAG ATCTACCAGG AGCTGACCCG GCTGAAGGCT | |
| | GCAGTGGGTG AGCTTCCAGA GAAATCTAAG CAGCAGGAGA TCTACCAGGA | |
| | GCTGACCCAG CTGAAGGCTG CAGTGGAACG CCTGTGCCAC CCCTGTCCCT | |
| | GGGAATGGAC ATTCTTCCAA GGAAACTGTT ACTTCATGTC TAACTCCCAG | |
| | CGGAACTGGC ACGACTCCAT CACCGCCTGC AAAGAAGTGG GGGCCCAGCT | |
| | CGTCGTAATC AAAAGTGCTG AGGAGCAGAA CTTCCTACAG CTGCAGTCTT | |
| | CCAGAAGTAA CCGCTTCACC TGGATGGGAC TTTCAGATCT AAATCAGGAA | |
| | GGCACGTGGC AATGGGTGGA CGGCTCACCT CTGTTGCCCA GCTTCAAGCA | |
| | GTATTGGAAC AGAGGAGAGC CCAACAACGT TGGGGAGGAA GACTGCGCGG | |
| | AATTTAGTGG CAATGGCTGG AACGACGACA AATGTAATCT TGCCAAATTC | |
| | TGGATCTGCA AAAAGTCCGC AGCCTCCTGC TCCAGGGATG AAGAACAGTT | |
| | TCTTTCTCCA GCCCCTGCCA CCCCAAACCC CCCTCCTGCG | |
| ECD AA | KV PSSISQEQSR QDAIYQNLTQ LKAAVGELSE | SEQ ID |
| | KSKLQEIYQE LTQLKAAVGE LPEKSKLQEI YQELTRLKAA VGELPEKSKL | NO: 307 |
| | QEIYQELTWL KAAVGELPEK SKMQEIYQEL TRLKAAVGEL PEKSKQQEIY | |
| | QELTRLKAAV GELPEKSKQQ EIYQELTRLK AAVGELPEKS KQQEIYQELT | |
| | QLKAAVERLC HPCPWEWTFF QGNCYFMSNS QRNWHDSITA CKEVGAQLVV | |
| | IKSAEEQNFL QLQSSRSNRF TWMGLSDLNQ EGTWQWVDGS PLLPSFKQYW | |
| | NRGEPNNVGE EDCAEFSGNG WNDDKCNLAK FWICKKSAAS CSRDEEQFLS | |
| | PAPATPNPPP A | |
| ECD DNA | AAGGTCCCCA GCTCCATAAG TCAGGAACAA TCCAGGCAAG ACGCGATCTA | SEQ ID |
| | CCAGAACCTG ACCCAGCTTA AAGCTGCAGT GGGTGAGCTC TCAGAGAAAT | NO: 308 |
| | CCAAGCTGCA GGAGATCTAC CAGGAGCTGA CCCAGCTGAA GGCTGCAGTG | |
| | GGTGAGCTTC CAGAGAAATC TAAGCTGCAG GAGATCTACC AGGAGCTGAC | |
| | CCGGCTGAAG GCTGCAGTGG GTGAGCTTCC AGAGAAATCT AAGCTGCAGG | |
| | AGATCTACCA GGAGCTGACC TGGCTGAAGG CTGCAGTGGG TGAGCTTCCA | |
| | GAGAAATCTA AGATGCAGGA GATCTACCAG GAGCTGACTC GGCTGAAGGC | |
| | TGCAGTGGGT GAGCTTCCAG AGAAATCTAA GCAGCAGGAG ATCTACCAGG | |
| | AGCTGACCCG GCTGAAGGCT GCAGTGGGTG AGCTTCCAGA GAAATCTAAG | |
| | CAGCAGGAGA TCTACCAGGA GCTGACCCGG CTGAAGGCTG CAGTGGGTGA | |
| | GCTTCCAGAG AAATCTAAGC AGCAGGAGAT CTACCAGGAG CTGACCCAGC | |
| | TGAAGGCTGC AGTGGAACGC CTGTGCCACC CCTGTCCCTG GGAATGGACA | |
| | TTCTTCCAAG GAAACTGTTA CTTCATGTCT AACTCCCAGC GGAACTGGCA | |
| | CGACTCCATC ACCGCCTGCA AAGAAGTGGG GGCCCAGCTC GTCGTAATCA | |
| | AAAGTGCTGA GGAGCAGAAC TTCCTACAGC TGCAGTCTTC CAGAAGTAAC | |
| | CGCTTCACCT GGATGGGACT TTCAGATCTA AATCAGGAAG GCACGTGGCA | |
| | ATGGGTGGAC GGCTCACCTC TGTTGCCCAG CTTCAAGCAG TATTGGAACA | |
| | GAGGAGAGCC CAACAACGTT GGGGAGGAAG ACTGCGCGGA ATTTAGTGGC | |
| | AATGGCTGGA ACGACGACAA ATGTAATCTT GCCAAATTCT GGATCTGCAA | |
| | AAAGTCCGCA GCCTCCTGCT CCAGGGATGA AGAACAGTTT CTTTCTCCAG | |
| | CCCCTGCCAC CCCAAACCCC CCTCCTGCG | |
| CRD AA | ER LCHPCPWEWT FFQGNCYFMS NSQRNWHDSI | SEQ ID |
| | TACKEVGAQL VVIKSAEEQN FLQLQSSRSN RFTWMGLSDL NQEGTWQWVD | NO: 309 |
| | GSPLLPSFKQ YWNRGEPNNV GEEDCAEFSG NGWNDDKCNL AKFWICKKSA | |
| | ASCSRDEEQF LSPAPATPNP PPA | |
| CRD DNA | GAACGCCTGT GCCACCCCTG TCCCTGGGAA TGGACATTCT TCCAAGGAAA | SEQ ID |
| | CTGTTACTTC ATGTCTAACT CCCAGCGGAA CTGGCACGAC TCCATCACCG | NO: 310 |
| | CCTGCAAAGA AGTGGGGGCC CAGCTCGTCG TAATCAAAAG TGCTGAGGAG | |
| | CAGAACTTCC TACAGCTGCA GTCTTCCAGA AGTAACCGCT TCACCTGGAT | |
| | GGGACTTTCA GATCTAAATC AGGAAGGCAC GTGGCAATGG GTGGACGGCT | |
| | CACCTCTGTT GCCCAGCTTC AAGCAGTATT GGAACAGAGG AGAGCCCAAC | |
| | AACGTTGGGG AGGAAGACTG CGCGGAATTT AGTGGCAATG GCTGGAACGA | |
| | CGACAAATGT AATCTTGCCA AATTCTGGAT CTGCAAAAAG TCCGCAGCCT | |
| | CCTGCTCCAG GGATGAAGAA CAGTTTCTTT CTCCAGCCCC TGCCACCCCA | |
| | AACCCTCCTC CTGCG | |
| Cyno DC-SIGN | | |
| Full length AA | MSDSKEPRLQ QLDLLEEEQL GGVGFRQTRG YKSLAGCLGH GPLVLQLLSF | SEQ ID |
| | TLLAGLLVQV SKVPSSLSQG QSKQDAIYQN LTQLKVAVSE LSEKSKQQEI | NO: 311 |
| | YQELTRLKAA VGELPEKSKQ QEIYEELTRL KAAVGELPEK SKLQEIYQEL | |
| | TRLKAAVGEL PEKSKQQEIY QELSRLKAAV GDLPEKSKQQ EIYQKLTQLK | |
| | AAVDGLPDRS KQQEIYQELI QLKAAVDLEG WTDTGIWTTS SEPSPDRPPP | |
| | TERLCHPCPW EWTFFQGNCY FMSNSQRNWH DSITACQEVG AQLVVIKSAE | |
| | EQNFLQLQSS RSNRFTWMGL SDLNHEGTWQ WVDGSPLLPS FKQYWNKGEP | |
| | NNVGEEDCAE FSGNGWNDDK CNLAKFWICK KSAASCSGDE ERLLSPAPTT | |
| | PNPPPE | |
| Full length | atgtcggactcgaaggaaccaagactgcagcaactcgacctccttgaagaagaacagctcgg | SEQ ID NO: 312 |
| DNA | cggagtgggattccggcagaccaggggttacaagagcctggccggttgcctgggtcacggccc | |
| | tttggtgcttcagctgctgtcgttcaccctgctggccggactgcttgtgcaagtctccaaagtccc | |
| | gtcctcgctgagccaggggcagtccaagcaggacgcgatctaccaaaacctgacacagctca | |
| | aggtggccgtgtcagagctgtccgagaagtcgaagcagcaagagatctaccaagagttgacg | |
| | cgactcaaagcagccgtgggcgaacttcccgagaagtcaaagcagcaggaaatctacgagg | |
| | aattgacccgcctgaaggccgccgtgggagagctgccagaaaagtcgaagctgcaggagata | |
| | taccaagaactcacccggctcaaggccgctgtgggagaactgccggagaagtccaaacaaca | |
| | ggaaatctaccaggaactgagcagactcaaggcagccgtcggcgatctccccgaaaagtcta | |
| | aacagcaggagatctatcagaagctgactcagctgaaggcggccgtggacgggctgcccgat | |
| | cggtccaagcaacaggaaatctaccaggagctgatccaactgaaggctgccgtggacctgga | |
| | agggtggactgacaccgggatttggactacctcatcggaaccgagccctgatcgccctccgcct | |
| | accgagaggttgtgtcacccgtgcccatgggagtggacgttcttccaaggaaactgttactttat | |
| | gagcaacagccagcggaattggcacgattccattaccgcgtgccaggaagtgggcgcccagc | |
| | tggtcgtgatcaagtccgcggaggagcagaacttcctgcagctccagagcagccggtccaacc | |
| | gcttcacctggatgggcctctccgacctgaaccatgagggaacttggcagtgggtggacggttc | |
| | cccgctgctgccctcattcaagcagtactggaacaagggagaaccgaacaacgtcggagagg | |
| | aagattgcgccgagttttccgggaacggatggaacgacgacaagtgcaatctggccaagttct | |
| | ggatttgcaagaagtccgctgcatcctgctcgggcgacgaggagcgcctgctgtcccccgcgc | |
| | ccaccacccctaaccctcccccggaatgatag | |
| ECD AA | QPSKQD AIYQNLTQLK VAVSELSEKS | SEQ ID |
| | KQQEIYQELT RLKAAVGELP EKSKQQEIYE ELTRLKAAVG ELPEKSKLQE | NO: 313 |
| | IYQELTRLKA AVGELPEKSK QQEIYQELSR LKAAVGDLPE KSKQQEIYQK | |
| | LTQLKAAVDG LPDRSKQQEI YQELIQLKAA VDLEGWTDTG IWTTSSEPSP | |
| | DRPPPTERLC HPCPWEWTFF QGNCYFMSNS QRNWHDSITA CQEVGAQLVV | |
| | IKSAEEQNFL QLQSSRSNRF TWMGLSDLNH EGTWQWVDGS PLLPSFKQYW | |
| | NKGEPNNVGE EDCAEFSGNG WNDDKCNLAK FWICKKSAAS CSGDEERLLS | |
| | PAPTTPNPPP | |
| ECD DNA | TC CAAGCAGGAC GCGATCTACC | SEQ ID |
| | AAAACCTGAC ACAGCTCAAG GTGGCCGTGT CAGAGCTGTC CGAGAAGTCG | NO: 314 |
| | AAGCAGCAAG AGATCTACCA AGAGTTGACG CGACTCAAAG CAGCCGTGGG | |
| | CGAACTTCCC GAGAAGTCAA AGCAGCAGGA AATCTACGAG GAATTGACCC | |
| | GCCTGAAGGC CGCCGTGGGA GAGCTGCCAG AAAAGTCGAA GCTGCAGGAG | |
| | ATATACCAAG AACTCACCCG GCTCAAGGCC GCTGTGGGAG AACTGCCGGA | |
| | GAAGTCCAAA CAACAGGAAA TCTACCAGGA ACTGAGCAGA CTCAAGGCAG | |
| | CCGTCGGCGA TCTCCCCGAA AAGTCTAAAC AGCAGGAGAT CTATCAGAAG | |
| | CTGACTCAGC TGAAGGCGGC CGTGGACGGG CTGCCCGATC GGTCCAAGCA | |
| | ACAGGAAATC TACCAGGAGC TGATCCAACT GAAGGCTGCC GTGGACCTGG | |
| | AAGGGTGGAC TGACACCGGG ATTTGGACTA CCTCATCGGA ACCGAGCCCT | |
| | GATCGCCCTC CGCCTACCGA GAGGTTGTGT CACCCGTGCC CATGGGAGTG | |
| | GACGTTCTTC CAAGGAAACT GTTACTTTAT GAGCAACAGC CAGCGGAATT | |
| | GGCACGATTC CATTACCGCG TGCCAGGAAG TGGGCGCCCA GCTGGTCGTG | |
| | ATCAAGTCCG CGGAGGAGCA GAACTTCCTG CAGCTCCAGA GCAGCCGGTC | |
| | CAACCGCTTC ACCTGGATGG GCCTCTCCGA CCTGAACCAT GAGGGAACTT | |
| | GGCAGTGGGT GGACGGTTCC CCGCTGCTGC CCTCATTCAA GCAGTACTGG | |
| | AACAAGGGAG AACCGAACAA CGTCGGAGAG GAAGATTGCG CCGAGTTTTC | |
| | CGGGAACGGA TGGAACGACG ACAAGTGCAA TCTGGCCAAG TTCTGGATTT | |
| | GCAAGAAGTC CGCTGCATCC TGCTCGGGCG ACGAGGAGCG CCTGCTGTCC | |
| | CCCGCGCCCA CCACCCCTAA CCCTCCCCCG GAA | |
| CRD AA | QPERLC HPCPWEWTFF QGNCYFMSNS | SEQ ID |
| | QRNWHDSITA CQEVGAQLVV IKSAEEQNFL QLQSSRSNRF TWMGLSDLNH | NO: 315 |
| | EGTWQWVDGS PLLPSFKQYW NKGEPNNVGE EDCAEFSGNG WNDDKCNLAK | |
| | FWICKKSAAS CSGDEERLLS PAPTTPNPPP E | |
| CRD DNA | GA GAGGTTGTGT CACCCGTGCC | SEQ ID |
| | CATGGGAGTG GACGTTCTTC CAAGGAAACT GTTACTTTAT GAGCAACAGC | NO: 316 |
| | CAGCGGAATT GGCACGATTC CATTACCGCG TGCCAGGAAG TGGGCGCCCA | |
| | GCTGGTCGTG ATCAAGTCCG CGGAGGAGCA GAACTTCCTG CAGCTCCAGA | |
| | GCAGCCGGTC CAACCGCTTC ACCTGGATGG GCCTCTCCGA CCTGAACCAT | |
| | GAGGGAACTT GGCAGTGGGT GGACGGTTCC CCGCTGCTGC CCTCATTCAA | |
| | GCAGTACTGG AACAAGGGAG AACCGAACAA CGTCGGAGAG GAAGATTGCG | |
| | CCGAGTTTTC CGGGAACGGA TGGAACGACG ACAAGTGCAA TCTGGCCAAG | |
| | TTCTGGATTT GCAAGAAGTC CGCTGCATCC TGCTCGGGCG ACGAGGAGCG | |
| | CCTGCTGTCC CCCGCGCCCA CCACCCCTAA CCCTCCCCCG GAA | |
| Human DC- | KV PSSISQEQSR QDAIYQNLTQ LKAAVGELSE | SEQ ID |
| SIGN ECD- | KSKLQEIYQE LTQLKAAVGE LPEKSKLQEI YQELTRLKAA VGELPEKSKL | NO: 317 |
| AviHis | QEIYQELTWL KAAVGELPEK SKMQEIYQEL TRLKAAVGEL PEKSKQQEIY | |
| | QELTRLKAAV GELPEKSKQQ EIYQELTRLK AAVGELPEKS KQQEIYQELT | |
| | QLKAAVERLC HPCPWEWTFF QGNCYFMSNS QRNWHDSITA CKEVGAQLVV | |
| | IKSAEEQNFL QLQSSRSNRF TWMGLSDLNQ EGTWQWVDGS PLLPSFKQYW | |
| | NRGEPNNVGE EDCAEFSGNG WNDDKCNLAK FWICKKSAAS CSRDEEQFLS | |
| | PAPATPNPPP AGSGGGLNDI FEAQKIEWHE HHHHHH | |
| Human DC- | MQLLSCIALS LALVTNSTER LCHPCPWEWT FFQGNCYFMS NSQRNWHDSI | SEQ ID |
| SIGN CRD- | TACKEVGAQL VVIKSAEEQN FLQLQSSRSN RFTWMGLSDL NQEGTWQWVD | NO: 318 |
| AviHis | GSPLLPSFKQ YWNRGEPNNV GEEDCAEFSG NGWNDDKCNL AKFWICKKSA | |
| | ASCSRDEEQF LSPAPATPNP PPAGSGGGLN DIFEAQKIEW HEHHHHHH | |
| Human DC- | MKTFILLLWV LLLWVIFLLP GATAQPSKVP SSISQEQSRQ DAIYQNLTQL | SEQ ID |
| SIGN ECD- | KAAVGELSEK SKLQEIYQEL TQLKAAVGEL PEKSKLQEIY QELTRLKAAV | NO: 319 |
| FLAGHis | GELPEKSKLQ EIYQELTWLK AAVGELPEKS KMQEIYQELT RLKAAVGELP | |
| | EKSKQQEIYQ ELTRLKAAVG ELPEKSKQQE IYQELTRLKA AVGELPEKSK | |
| | QQEIYQELTQ LKAAVERLCH PCPWEWTFFQ GNCYFMSNSQ RNWHDSITAC | |
| | KEVGAQLVVI KSAEEQNFLQ LQSSRSNRFT WMGLSDLNQE GTWQWVDGSP | |
| | LLPSFKQYWN RGEPNNVGEE DCAEFSGNGW NDDKCNLAKF WICKKSAASC | |
| | SRDEEQFLSP APATPNPPPA DYKDDDDKHH HHHH | |

### Generation of Cell Lines Stably Expressing DC-SIGN

Stable full length DC-SIGN-expressing full length L-SIGN expressing K562 cell lines were generated using retroviral transduction. HEK293T cells were co-transfected with a DC-SIGN retroviral expression vector and a pCL-10A1 packaging vector (Novus, USA, cat #NBP2-2942) using Fugene 6 transfection reagent (Promega, USA, cat #E2692) following manufacturer's recommendation. Cells were incubated in a 37° C. humidified 002 incubator and viral supernatant was collected 48 hours post-transfection. K562 cells were grown to near confluency. Viral transduction was performed by adding viral supernatant in the presence of 8 μg polybrene/ml (final concentration) (EMD Millipore, cat #TR-1003-G). Following incubation for 3-6 hours at 37° C., fresh media was added. Cells were then cultured under appropriate selection conditions to produce stable L-SIGN or DC-SIGN expressing cell lines.

Stable human DC-SIGN expressing and cynomolgus monkey DC-SIGN expressing CHO cell lines were generated using plasmid DNA. Proprietary CHO cells were nucleoporated with a human or cynomolgus monkey DC-SIGN gene in the pD649 expression vector (DNA2.0). Nucleoporation was performed using the Lonza SG Cell line 96-well Nucleoporation kit (Cat #V4SC-3096). Cells and plasmid DNA were mixed with SG buffer and supplement, following manufacturer's recommendation. The 96-well nucleoporation plate was placed in a Nucleofector™ 96-well Shuttle™ (Lonza) and processed using program CHO S (FF-137). Nucleoporated cells were allowed to sit for 30 min at RT before diluting. Viability and cell density measurements were performed using VICELL (Beckman Coulter). Cells were seeded into a 96-well plate at 40,000 cells/well into 100 μL of proprietary DM122 media and incubated at 37° C., 10% CO₂ at 4 hrs after seeding, selection was added to the cells (4 μg/mL of puromycin (InvivoGen) for cynomolgus monkey and 100 nM methotrexate (Sigma) for human DC-SIGN). Every 7 days, cells were passed 1:5 into fresh selection media for 3 passages. Cells were expanded into shake flasks at 37° C., 10% CO₂ and kept at densities 0.1million cells/mL to 2 million cells/mL. After 4 weeks, cells were FACS sorted using a 2008 FACS Aria to obtain cell pools with high expression levels for both cell lines.

### Hybridoma Generation, Antibodies 2B2 and 1G12

Bcl-2 transgenic mice (C₅₇BL/6-Tgn (bcl-2) 22 WEHI strain) were immunized with antigen using a procedure that calls for Repetitive Immunization at Multiple Sites (RIMMS) (Kilpatrick K E, et al., Hybridoma 16(4):381-9 (1997)). Briefly, mice were injected with 1-3 μg of DC-SIGN immunogen (Recombinant Human DC-SIGN/CD209 Fc Chimera Protein, CF, R&D systems Cat No: 161-DC-050) at 8 specific sites proximal to peripheral lymph nodes (PLNs). This procedure was repeated 8 times over a 12 day period. On Day 12, a test bleed was collected and the serum antibody titer was analyzed by FACS. Two days after the boost, a test bleed was collected and serum antibody titer was analyzed by FACS. In some instances, BALB/c mice were immunized subcutaneously with antigen once a month for 3 months followed by an intravenous boost. Two days after the boost, a test bleed was collected and serum antibody titer was analyzed by FACS. Spleens and pooled PLNs were removed from high titer mice. To harvest lymphocytes, spleens and PLNs were washed twice with DMEM, and then dissociated by passage through a 70 micron screen (Falcon #352350). The resulting lymphocytes were washed 2 additional times prior to fusion in Cytofusion media (BTXpress Cytofusion® Electroporation Medium cat #47001).

Ten days after fusion, hybridoma plates were screened for the presence of human DC-SIGN-specific antibodies using flow cytometry. To confirm specific binding of candidate antibodies to cell surface-expressed human DC-SIGN, three cell lines were used: human DC-SIGN stably overexpressing K562, human L-SIGN stably overexpressing K562 or parental K562. Cells were rinsed thoroughly with PBS. Cells were biotinylated and labeled with a fluorescent dye according to manufacturer's instructions (FluoReporter™ Cell-Surface Biotinylation Kit, Thermo Fisher Scientific Cat #F-20650; PE-Cy7 Streptavidin, ThermoFisher Scientific Cat #SA1012; APC Streptavidin, Biolegend Cat #405207; APC/Cy7 Streptavidin, Biolegend Cat #405208). Cells were resuspended at approximately 1×106 cells/ml in FACS buffer (PBS with 2% FBS+0.1% NaN3). In a 384-well plate, 20 μL of hybridoma supernatant was pre-seeded, and 20 μL of cell suspension was added. Cells were incubated for 1 hour at 4° C., washed twice with cold FACS buffer, and resuspended in 20 μL of FACS buffer containing secondary antibody at a 1:400 dilution (Goat anti-mouse IgG BV421, Sirigen, custom order). After additional incubation for 45 min at 40C, cells were washed twice with FACS buffer and resuspended in 20 μL of FACS buffer with 2 μg/ml propidium iodide (Sigma Aldrich Cat #P4864). Geometric mean fluorescence intensity was calculated on live single cells using FlowJo™ software.

### Hybridoma Generation 2, Antibodies 960K03, 958N02, 956P16, 952G04, 952D15, 914M09, 906C18, 956E02, 550E03, 942K11

Ablexis Alivamab Kappa (AMM-K) and Lambda (AMM-L) mice were immunized with antigen using a procedure that calls for Repetitive Immunization at Multiple Sites (RIMMS) (Kilpatrick K E, et al., Hybridoma 16(4):381-9 (1997)). Briefly, mice were injected with 22.5 μg of full length ECD-AviHis (SEQ ID NO: 317) protein at 8 specific sites proximal to peripheral lymph nodes (PLNs). This procedure was repeated 8 times over a 20 day period. On Day 18, a test bleed was collected and the serum antibody titer was analyzed by FACS and ELISA prior to hybridoma fusion. To harvest lymphocytes, spleens and lymph nodes were mechanically dissociated in PBS, and then passaged through a 70 micron screen (Falcon #352350). RBCs were lysed using Red Blood Cell Lysing Buffer (SigmaR7757-100 ml) as per manufacturer's instructions. CD3 positive splenocytes were removed using micro bead magnetic columns from Miltenyi as per their instructions (Anti-IgM #130-047-301 and anti-CD3 #130-094-973). The resulting lymphocytes were washed 2 additional times prior to fusion in Electrofusion IsoOsmolar Buffer (Eppendorf, #4308 070 536).

For the fusion, FO myeloma cells were mixed with lymphocytes at a 1:4 ratio. The cell mixture was centrifuged, suspended in Electrofusion IsoOsmolar Buffer and subsequently added to an electrofusion chamber (Harvard Apparatus Coaxial chamber 9ML Part #470020). Electrofusion was carried out per manufacturer's instructions using the CEEF-50B Hybrimune/Hybridoma system (Cyto Pulse Sciences, Inc). Fused cells were allowed to recover for 5 minutes in the chamber, diluted 1:10 in media without hypoxanthine-aminopterin-thymidine (HAT) [DMEM+20% FBS, 1% Penicillin-Streptomycin-Glutamine (PSG), 1× Non-Essential Amino Acids (NEAA), 0.5× Hybridoma Fusion and Cloning Supplement (Roche; HFCS) and placed at 37° C. and 5% CO₂ for one hour. Next, 4×HAT medium (DMEM+20% FBS, 1% PSG, 1×NEAA, 4×HAT, 0.5×HFCS) was added to bring the concentration of HAT to 1×, and the density was adjusted to 66,000 cells/ml. The cells were plated in 384-well plates at 60 μl/well.

### FACS Screening

Ten days after fusion, hybridoma plates were screened for the presence of human DC-SIGN-specific antibodies using flow cytometry. To confirm specific binding of candidate antibodies to cell surface-expressed human DC-SIGN, three cell lines were used: human DC-SIGN stably overexpressing CHO, cynomolgus DC-SIGN stably overexpressing CHO, and parental non-transfected CHO cells. Cells were rinsed thoroughly with PBS. Cells were biotinylated and labeled with a fluorescent dye according to manufacturer's instructions (FluoReporter™ Cell-Surface Biotinylation Kit, Thermo Fisher Scientific Cat #F-20650; PE-Cy7 Streptavidin, ThermoFisher Scientific Cat #SA1012; APC Streptavidin, Biolegend Cat #405207; APC/Cy7 Streptavidin, Biolegend Cat #405208). Cells were resuspended at approximately 1×106 cells/ml in FACS buffer (PBS with 2% FBS+0.1% NaN3). In a 384-well plate, 20 μL of hybridoma supernatant was pre-seeded, and 20 μL of cell suspension was added. Cells were incubated for 1 hour at 4° C., washed twice with cold FACS buffer, and resuspended in 20 μL of FACS buffer containing secondary antibody at a 1:400 dilution (Goat anti-mouse IgG BV421, Sirigen, custom order). After additional incubation for 45 min at 4° C., cells were washed twice with FACS buffer and resuspended in 20 μL of FACS buffer with 2 μg/ml propidium iodide (Sigma Aldrich Cat #P4864). Geometric mean fluorescence intensity was calculated on live single cells using FlowJo™ software.

Hits from the primary cell-based flow cytometry screen were confirmed in a secondary flow cytometry screen like above, but with two additional cell lines: human DC-SIGNstably overexpressing K562 and human L-SIGN stably overexpressing K562 cells. Hybridomas expressing antibodies that bound to both human DC-SIGN expressing CHO and human DC-SIGN expressing K562 cells, but not CHO parental cells or L-SIGN-K562 cells, were called positive. Positive cells were expanded for cryo preservation and also split into 45 mL protein production cultures in hybridoma serum-free medium with HT Media Supplement (50×) Hybri-Max™ (Sigma, cat #H0137) in CellStar® Autoflasks™ (Greiner Bio-One). Production cultures were maintained in a shaking incubator at 37° C. and 5% CO₂ for approximately 8 days. Cells were then pelleted, and supernatants were taken through purification over Protein G resin. Proteins were subsequently buffer exchanged into PBS using NAP-10™ columns (GE Healthcare).

### Antibody Sequencing and Vector Preparation

Variable region (VH and VL) DNA sequences of hybridomas were obtained for each of the selected hybridomas. Variable region DNA products from murine monoclonal antibodies 2B2 and 1G12 were amplified by rapid amplification of cDNA ends (RACE) from RNA obtained from each selected hybridoma cell line using standard methods. Variable region DNA products from monoclonal antibodies 960K03, 958N02, 956P16, 952G04, 952D15, 914M09, 906C18, 956E02, 550E03, 942K11 were amplified by PCR from selected hybridoma cell line using standard methods and pooled primers to signal peptide and constant regions of the antibody genes.

For preparation of recombinant antibodies, DNA sequences coding for the hybridoma VL and VH domain were subcloned into expression vectors containing the respective human heavy or light chain constant region sequences (IgG1, kappa). In some instances this resulted in chimeric antibody chains comprising a murine variable region and human constant region. In some instances this resulted in fully human antibody sequence. In some instances, expression vectors contained wild type human constant region sequences. In some instances, expression vectors contained human constant region sequences comprising site-specific cysteine mutations as has been described previously in WO 2014/124316 and WO 2015/138615. For example, cysteines were introduced at one or more of the following positions (all positions by EU numbering) in an anti-DC-SIGN antibody: (a) positions 152 and/or 375 of the antibody heavy chain, and (b) position 165 of the antibody light chain. In some instances, constant region sequences comprise mutations known in the art to alter binding to Fc-receptors (e.g., D265A/P329A mutations in the heavy chain) to include constructs having reduced Fc effector function. In some instances, expression vectors contain constant regions comprising combinations of the modifications described above. In some instances, expression vectors contained mouse constant region sequences (IgG2a, kappa), either wild-type or with one or more mutations analagous to those described above (e.g. E152C, A375C, D265A, P329A), resulting in fully mouse antibody sequences. Heavy and light chains were cloned into individual expression vectors to allow co-transfection.

### Humanization of Antibodies 2B2 and 1G12

Variable region constructs were designed for humanization and optimization of sequences (e.g., removal of post-translational modifications, non-preferred sites, etc.).

Corresponding DNA sequences coding for humanized VL and VH domains were ordered at GeneArt (Life Technologies Inc. Regensburg, Germany), including codon optimization for Cricetulus griseus. Sequences coding for VL and VH domains were subcloned from the GeneArt derived vectors into expression vectors suitable for protein production in mammalian cells as described above for parental sequences. In some instances, the expression vector for the heavy chain comprised a truncation resulting in expression of a Fab fragment, and in some instances this constant region sequence was modified with a site-specific cysteine mutation at position 152 as described above, and additionally in some instances there was a sequence encoding a His-tag fused to the C-terminus of the Fab heavy chain coding sequence. Heavy and light chains were cloned into individual expression vectors to allow co-transfection.

### Optimization of Antibodies 960K03, 958N02, 956P16, 952G04, 952D15

Variable region constructs were designed for optimization of sequences by removal of post-translational modifications, non-preferred sites etc. Substitutions were made by site directed mutagenesis using standard methods. Heavy and light chains were cloned into individual expression vectors to allow co-transfection.

### Antibody Production

Recombinant antibodies (IgG1, kappa) were produced by co-transfection of heavy chain and light chain vectors into Freestyle™ 293 expression cells (Invitrogen, USA) using standard methods known in the art and similar to those described previously in Meissner, et al., Biotechnol Bioeng. 75:197-203 (2001).

Following transfection, the cells were cultured for one to two weeks prior to antibody purification from supernatant.

Alternatively, recombinant antibodies were produced by co-transfection of heavy chain and light chain vectors into CHO cells using methods known in the art. Following transfection, the cells were kept in culture for up to two weeks prior to antibody purification from supernatant.

To generate stable cell lines for antibody production, vectors were co-transfected by nucleofection (Nucleofector™ 96-well Shuttle™; Lonza) into CHO cells using manufacturer's recommendations, and cultured under selection conditions for up to four weeks in shake flasks. Cells were harvested by centrifugation, and supernatant recovered for antibody purification.

Antibodies and antibody fragments were purified using Pprotein A, Protein G or MabSelect SuRe (GE Healthcare Life Sciences) columns. Prior to loading the supernatant, the resin was equilibrated with PBS. Following binding of the sample, the column was washed with PBS, and the antibody was eluted with Thermo (Pierce) IgG Elution Buffer pH 2.8 (cat #21004). The eluate fractions were neutralized with sodium citrate tribasic dehydrate buffer, pH 8.5 (Sigma Aldrich cat #S4641-1Kg). Buffer exchange was performed by dialyzing overnight or by NAP-10™ columns (GE Healthcare), typically into PBS, pH 7.2. In some instances, antibodies may be further purified. One example is to apply the antibody to a size exclusion chromatography (SEC) column such as one with Superdex™ 200 resin (GE Healthcare) and collect the peak corresponding to the monomer species.

### Summary of Antibodies

Table 1 sets forth the relevant sequence information for parental and humanized anti-DC-SIGN antibodies derived from murine hybridomas. Throughout this application, when describing the antibodies, the term “Hybridoma” is used interchangeably and can refer to the antibody that is derived from the hybridoma.

### Example 73: Biochemical Characterization of Antibodies

### Affinities of Anti-DC-SIGN Antibodies to DC-SIGN

The affinity of various antibodies and ADCs to DC-SIGN and its species orthologues was determined using FACS. Purified IgGs were titrated to determine EC50 values for binding to cell surface expressed DC-SIGN.

For this purpose, human DC-SIGN expressing or cynomolgus monkey DC-SIGN expressing stable CHO cell lines or K562 expressing DC-SIGN or K562 expressing L-SIGN cell lines were checked for density and viability using VICELL (Beckman Coulter), and washed once with 4° C. PBS. Cells were stained with DAPI (0.5 ug/mL) diluted in PBS for 30 min on ice. Cells were diluted into 4° C. FACS buffer (PBS, 10 mM EDTA, 2% FBS). 125 μl of cells were seeded (10,000 cells/well) into 96-well v-bottom plates (Nunc cat #442587) and centrifuged for 4 min at 1500 rpm at 4° C. Supernatant was removed. Cells were incubated with a serial dilution of each anti-DC-SIGN antibody in FACS buffer at concentrations ranging across several logs with a top concentration no higher than 50 μg/mL for 60 minutes at 4° C. Following incubation, cells were spun down (1500 rpm, 4 min, 4° C.) and washed two times with FACS buffer. A fluorophore-conjugated anti-hFc gamma-AF-647 (Southern Biotechnology) detection antibody was added at 1:400 dilution and samples were incubated for 1 h on ice in the dark. Following incubation, FACS buffer was added, and the cells were spun down (1500 rpm, 4 min, 4° C.) and washed two times with FACS buffer. After the final wash, cells were resuspended in Fixative Buffer (Biolegend, 420801) and 90 μl of FACS buffer followed by readout on the flow cytometry machine (BD LSRFortessa Cell Analyzer; Cat #647177). Geometric Mean fluorescence intensity (MFI) of live, single cells was calculated in Flowjo 10.4.2 and exported into Graphpad Prism7 for EC50 determination.

Selectivity was assessed by measuring apparent binding affinities to isogenic cell pairs engineered to overexpress DC-SIGN as well as cell lines expressing DC-SIGN paralog L-SIGN. Anti-DC-SIGN antibodies bind in a specific manner to DC-SIGN expressing cells only, as shown in Table 16 below.

In a similar experiment the antibodies were tested for cross-reactivity using engineered isogenic matched cell line. All antibodies except 892D15 and 942K11 were found to specifically bind human and cynomolgus monkey DC-SIGN at similar apparent affinities, as shown in Table 16 below.

TABLE 16
Binding of Various Anti-DC-SIGN Antibodies
to DC-SIGN and L-SIGN Expressing Cells

| | human | cyno | human | human |
|---|---|---|---|---|
| | DC-SIGN | DC-SIGN | DC-SIGN | L-SIGN |
| | CHO | CHO | K562 | K562 |
| Antibody | EC50 | EC50 | EC50 | EC50 |
| Name | (ug/mL) ave. | (ug/mL) ave. | (ug/mL) | (ug/mL) |
| 2B2 Hz | 0.06 | 0.04 | 0.27 | >10 |
| 960K03 N92S | 0.06 | 0.08 | 0.049 | |
| 958N05 S93A | 0.13 | 0.16 | 0.060 | >10 |
| 960K03 N92Q | 0.08 | 0.04 | 0.021 | >10 |
| 952P16 N92Q | 0.06 | 0.04 | 0.024 | >10 |
| 952G04 N92Q | 0.07 | 0.02 | 0.017 | >10 |
| 2B2 Chimeric | 0.23 | 0.32 | 0.28 | >10 |
| 960K03 Parental | 0.10 | 0.02 | | |
| 958N05 Parental | 0.07 | 0.08 | 0.05 | |
| 952P16 Parental | 0.06 | 0.02 | 0.04 | |
| 952G04 Parental | 0.11 | 0.19 | 0.26 | |
| 892D15 Parental | 0.15 | 15.69 | >10 | |
| 914M09 Parental | 0.10 | 0.08 | 0.25 | >10 |
| 906C18 Parental | 0.21 | 0.77 | 1.72 | >10 |
| 956 E02 Parental | 0.12 | 0.13 | | |
| 942K11 Parental | 0.20 | 11.57 | | |
| 550 E03 Parental | 0.26 | 0.39 | 1.50 | >10 |
| 1G12 Hz | 0.07 | 0.06 | 0.021 | >10 |
| 1G12 mouse | | | | |
| 1G12 Parental | 0.06 | 0.07 | 0.02 | >10 |

### Affinities of Anti-DC-SIGN Antibodies to DC-SIGN

The affinity of various antibodies to DC-SIGN Carbohydrate Recognition Domain (CRD) was determined using Biacore. Purified IgGs for the parental antibodies were titrated to determine Kd values for binding to purified antigen domain by two methods described below.

In method 1 DC-SIGN was used as the ligand (surface attached) and the antibody the analyte (injected at different concentrations). The DC-SIGN CRD was captured via the His tag on a CM5 chip that was prepared by immobilizing 12000 R U NeutrAvidin followed by capturing ˜550 RU of Tris-NTA biotin. Fresh DC-SIGN was used for each dose. Each cycle consisted of charging the surface with a 120 s pulse of 5 mM NiCl₂, capturing the same amount of DC-SIGN, injecting the antibody at the desired concentration, and stripping the Ni²⁺ with pulses of 350 mM EDTA and 500 mM imidazole to remove all DC-SIGN. Antibodies were injected at concentrations between 250 and 31 nM for 180 s and allowed to dissociate for 600s. The reverse orientation was used in method 2—antibody the ligand and DC-SIGN the analyte. A CM5 chip was first prepared with mouse anti-human IgG Fc and used to capture the antibodies. Fresh antibody was used for each dose where each cycle consisted of capturing the same amount of antibody (˜100 RU), injecting the desired concentration of DC-SIGN, and stripping the surface of all captured antibody with two 30 s pulses of 10 mM glycine pH 2.0. DC-SIGN was injected for 180 s at concentrations between 500 and 1.95 nM and dissociated for 600 s. All experiments were conducted on a GE Biacore 8K at 25° C. with a flow rate of 30 μL/min in 10 mM HEPES, 500 mM NaCl, 2.5 mM Imidazole, 0.05% Tween 20, pH 7.4. Kinetic parameters were calculated using the 8K analysis software.

TABLE 17
Binding of Various Anti-DC-SIGN Antibodies to DC-
SIGN Carbohydrate Recognition Domain by Biacore

| | | high density |
|---|---|---|
| | | DC-SIGN |
| | Ab on chip | CRD on chip |
| | (nM) | (nM) |
| 960 K03 | 955 | 0.8 |
| 906C18 | 1950 | 16 |
| 914M09 | 830 | 10 |
| 956 E02 | 999 | 13 |
| 942K11 | 1260 | 180 |
| 550Ee03 | 523 | 5.8 |
| 952P16 | 395 | 3.8 |
| 952G04 | 346 | 3.7 |
| 958N05 | 174 | 1.7 |
| 892D15 | 47600 | 111 |
| 2b2 | 32 | 0.5 |

### Epitope Binning Using Octet Red96 System

Epitope binning of anti-DC-SIGN parental antibodies was performed using the Octet Red96 system (ForteBio, USA) that measures biolayer interferometry (BLI). For this purpose the DC-SIGN extracellular domain with the AviHis tag (SEQ ID NO: 317) was biotinylated via an AviTag™ utilizing BirA biotin ligase according to Manufacturer's recommendations (Avidity, LLC, USA cat #BirA500). The biotinylated immunogen scaffold was loaded at 0.4 μg/ml onto pre-equilibrated streptavidin sensors (ForteBio, USA). The sensors were then transferred to a solution containing 100 nM antibody A in 1× kinetics buffer (ForteBio, USA). Sensors were briefly washed in 1× kinetics buffer and transferred to a second solution containing 33.3 nM of competitor antibody B. Binding kinetics parameters were determined from raw data using the Octet Red96 system analysis software (Version 6.3, ForteBio, USA). Antibodies were tested in all pairwise combinations, as both Antibody A and as competitor antibody B.

TABLE 18
Antibody Binning Results

| Bin | Antibody |
|---|---|
| 1 | 2B2, 892D15, 960K03, 906C18, 952P16, 942G04 |
| 2 | 914M09, 956E02 |
| 3 | 942K11 |

### Epitope Mapping Using Hydrogen/Deuterium Exchange Mass Spectrometry (HDxMS)

Additional epitope mapping was carried out for antibody 2B2 using HDxMS. DC-SIGN ECD (SEQ ID NO: 319) was concentrated 5× using a 10 kDa MWCO micro-concentrator. 5 μg of protein was used in each sample and DCSIGN ECD/mAb complexes were prepared by mixing an equimolar amount of DC-SIGN ECD (SEQ ID NO: 319) and each mAb separately. Complexes were allowed to form for 30 min. at room temp before labeling.

For non-deuterated, deuterated controls and deuterated complexes, each sample was diluted with the appropriate volume of labeling buffer (50 mM Phosphate buffer, pH 7.6 or pH 8.6, 150 mM NaCl in H₂O) to bring the total volume to 10 μL. Solutions were placed in 1.5 mL vials and placed in a rack at either 0° C. or 20° C. The labeling step for all samples was performed with the addition of 50 μL of labeling buffer (50 mM Phosphate buffer, pH 7.6 or 8.6, 150 mM NaCl in H₂O) to each sample. Solutions were incubated for 5 min. Vials were transferred to an ice water bath and 250 μL of reduction buffer (8M GndHCl, 1M TCEP, pH2.5) was added and mixed. After 2 min, 300 μL of ice cold quench buffer (0.25% formic acid, 12.5% glycerol) was added and the solutions were immediately frozen in liquid nitrogen. Vials were transferred to the −70° C. freezer attached to a PAL autosampler for HDx analysis. Samples were thawed for 2 min and 500 μL was injected through an in-line pepsin column into the LC-MS system. Proteolytic peptides were sequenced by tandem mass spectrometry (MS/MS) and deuteration values were extracted using HDExaminer.

TABLE 19
Antibody 2B2 protected exchange of the amide
hydrogens in the peptides with the sequences

| Peptide | | |
|---|---|---|
| protected | Amino acid sequence | |
| 1 | VVIKSAEEQNF | SEQ ID NO: 320 |
| 2 | LQLQSSRSNRFTWMGLSDL | SEQ ID NO: 321 |
| 3 | NQEGTWQWVDGSPLL | SEQ ID NO: 322 |
| 4 | NQEGTWQWVDGSPLLPSF | SEQ ID NO: 323 |

### Example 74

### Generation of Anti-DC-SIGN-TLR7 Agonist Conjugates by Conjugation of TLR7 Agonists to Specific Cysteine Residues of Anti-DC-SIGN Antibody Mutants

Preparation of Anti-DC-SIGN Antibody with Specific Cysteine (Cys) Mutations

Preparation of anti-DC-SIGN antibodies and other antibodies with site-specific cysteine mutations has been described previously in WO 2014/124316 and WO 2015/138615, each of which was incorporated by reference herein.

Reduction, Re-Oxidation and Conjugation of Cys Mutant Anti-DC-SIGN Antibodies to TLR7 Agonists

Some compounds described herein comprising a linker were conjugated to Cys residues engineered into an antibody similar to what is described in Junutula J R, et al., Nature Biotechnology 26:925-932 (2008).

Because engineered Cys residues in antibodies expressed in mammalian cells are modified by adducts (disulfides) such as glutathione (GSH) and/or cysteine during biosynthesis (Chen et al. 2009), the modified Cys as initially expressed is unreactive to thiol reactive reagents such as maleimido or bromo-acetamide or iodo-acetamide groups. To conjugate engineered Cys residues, glutathione or cysteine adducts need to be removed by reducing disulfides, which generally entails reducing all disulfides in the expressed antibody. This can be accomplished by first exposing antibody to a reducing agent such as cysteine followed by re-oxidation of all native disulfide bonds of the antibody to restore and/or stabilize the functional antibody structure. Cys mutant antibodies were reduced and re-oxidized using an on-resin method. Protein A Sepharose beads (1 mL per 10 mg antibody) were equilibrated in PBS (no calcium or magnesium salts) and then added to an antibody sample in batch mode. A stock of 0.5 M cysteine was prepared by dissolving 850 mg of cysteine HCl in 10 ml of a solution prepared by adding 3.4 g of NaOH to 250 mL of 0.5 M sodium phosphate pH 8.0 and then 20 mM cysteine was added to the antibody/bead slurry, and mixed gently at room temperature for 30-60 minutes. Beads were loaded to a gravity column and washed with 50 bed volumes of PBS in less than 30 minutes, then the column was capped with beads resuspended in one bed volume of PBS. To modulate the rate of re-oxidation, 50 nM to 1 μM copper chloride was optionally added. The re-oxidation progress was monitored by removing a small test sample of the resin, eluting in IgG Elution buffer (Thermo), and analyzing by RP-HPLC. Once re-oxidation progressed to desired completeness, conjugation could be initiated immediately by addition of 2-3 molar excess of compound over engineered cysteines, and allowing the mixture to react for 5-10 minutes at room temperature before the column was washed with at least 20 column volumes of PBS. Antibody conjugates were eluted with IgG elution buffer and neutralized with 0.1 volumes 0.5 M sodium phosphate pH 8.0. Conjugates were typically buffer exchanged to PBS pH 7.2 and analyzed by methods described below. In some instances, conjugates were further purified by standard preparative size exclusion chromatography methods.

Properties of the Anti-DC-SIGN-TLR7 Agonist Conjugates

Antibody-TLR7 agonist conjugates were analyzed to determine extent of conjugation. A compound-to-antibody ratio was extrapolated from LC-MS data for reduced and deglycosylated samples. LC/MS allows quantitation of the average number of molecules of linker-payload (compound) attached to an antibody in a conjugate sample. HPLC separates antibody into light and heavy chains, and separates heavy chain (HC) and light chain (LC) according to the number of linker-payload groups per chain. Mass spectral data enables identification of the component species in the mixture, e.g., LC, LC+1, LC+2, HC, HC+1, HC+2, etc. From the average loading on the LC and HC chains, the average compound to antibody ratio can be calculated for an antibody conjugate. A compound-to-antibody ratio for a given conjugate sample represents the average number of compound (linker-payload) molecules attached to a tetrameric antibody containing two light chains and two heavy chains.

Conjugates were profiled using analytical size-exclusion chromatography (AnSEC) on Zenix C-300 3 um 7.8×150 mm column (Sepax Technologies). Alternatively, samples were tested on a KW-803 column (TIC Cat #6960940) Aggregation was analyzed based on analytical size exclusion chromatography and reported as the % monomer based on AUC of the assigned monomer peak.

The sample shown below in Table 20 achieved >90% conjugation efficiency and >95% purity as assessed by AnSEC. This illustrates that conjugates of the invention can be made efficiently and have favorable characteristics.

In the Examples below, unless otherwise indicated, all anti-DC-SIGN-TLR7 agonist conjugates used were the DAR4 version.

TABLE 20
Properties of anti-DC-SIGN-TLR7 agonist conjugates

| | | | Compound- | Conjugation | |
|---|---|---|---|---|---|
| | Linker- | | to-antibody | Efficiency | % |
| Antibody | Payload | Payload | ratio | (%) | Monomer |
| 2B2 | C-5 | C-8a | 3.9 | 98 | 95.4/99^{a} |
| Chimeric | | | | | |

^{a}Values reported before and after preparative SEC.

### Reduction, Re-Oxidation and Conjugation of Hair-Pin RNA Payloads to Cys Mutant Antibodies

Because engineered Cys residues in antibodies expressed in mammalian cells are modified by adducts (disulfides) such as glutathione (GSH) and/or cysteine during biosynthesis, the modified Cys as initially expressed is unreactive to thiol reactive reagents such as maleimido or bromo-acetamide or iodo-acetamide groups. To conjugate engineered Cys residues, glutathione or cysteine adducts need to be removed by reducing disulfides, which generally entails reducing all disulfides in the expressed antibody. This can be accomplished by first exposing antibody to a reducing agent such as dithiothreitol (DTT) or TCEP or Cysteine followed by re-oxidation of all native disulfide bonds of the antibody to restore and/or stabilize the functional antibody structure. Accordingly, freshly prepared DTT is added to Cys mutant antibodies to a final concentration of 5 mM to 20 mM. After incubation of antibodies with DTT at 37° C. for 1 hour, DTT is removed by a gel filtration step. The antibody solutions are then allowed to reoxidize to reform native disulfide bonds. Alternatively Cys mutant antibodies are reduced and re-oxidized using an on-resin method. Antibody solutions are loaded to Protein A Sepharose resin columns (1 ml per 10 mg antibody). 20 mM to 100 mM cysteine solutions are added to the protein A column bound with antibodies, and incubated with the antibodies in the columns at room temperature for 30-60 minutes. The protein A columns are then washed with 50 bed volumes of PBS in less than 30 minutes, and resuspended in one bed volume of PBS. 50 nM to 1 μM of CuCl₂ is optionally added to the antibody-bound resins and the reoxidation of the antibodies is allowed to happen on resins.

The re-oxidation process are monitored by reverse-phase HPLC, which is able to separate intact antibody from individual heavy and light chain molecules. Reactions are analyzed on a PRLP-S 4000A column (50 mm×2.1 mm, Agilent) heated to 80° C. and column elution was carried out by a linear gradient of 30-60% acetonitrile in water containing 0.1% TFA at a flow rate of 1.5 ml/min. The elution of proteins from the column was monitored at 280 nm.

After reoxidation of antibodies is completed, a linker containing a maleimide group and an azide group is incubated with the reoxidized antibodies at room temperature for 30 min at a ratio of linker:Ab=10:1 in PBS (pH7.2). The linker conjugated antibodies are then purified by protein A column chromatography which removes non-conjugated linkers. After protein A column purification, the linker-conjugated antibodies are adjusted to PBS (pH8.0). Synthesized hair-pin RNA payloads containing an alkyne group are added to the solutions of linker conjugated antibodies (1 mg/ml) to a final concentration of 30 μM to 60 μM. The incubation is conducted at room temperature for 24 hours. In certain examples, 10 μM to 100 μM CuSO₄ and 50 μM to 500 μM of BTTAA (bis[(tertbutyltriazoyl)methyl]-[(2-carboxymethyltriazoyl)methyl]-amine) or THPTA (Tris(3-hydroxypropyltriazolyl-methyl)amine]) are added to the incubation mixtures to accelerate conjugation reactions. After the conjugation reactions the antibody conjugation mixtures are loaded to protein A columns and non-conjugated hair-pin RNA payloads are removed by washing the columns with PBS. The payload conjugated antibodies are eluted and adjusted to PBS (pH7.5).

### Example 75

### 2B2 (DAPA)C-5 Conjugate is Active on Human Monocyte DCs and Macrophages In Vitro

Primary human monocytes were isolated from a leukapheresis using magnetic bead selection and frozen for storage in liquid nitrogen. For monocyte DC (moDC) differentiation, cells were thawed and incubated in media containing GM-CSF and IL-4 for 7 days. For M2 macrophages (M2 moMacs), cells were thawed and incubated for 6 days with M-CSF containing media and then polarized with the addition of IL-4 for 24 hours. After the differentiation process for both moDC and moMacs, media was washed off and replaced with fresh media containing isotype control (DAPA)C-5, or 2B2 (DAPA) C5 conjugate. Free C-8a compound was used as a control. 24 hours after incubation with indicated compounds, cells were evaluated by flow cytometry for activation.

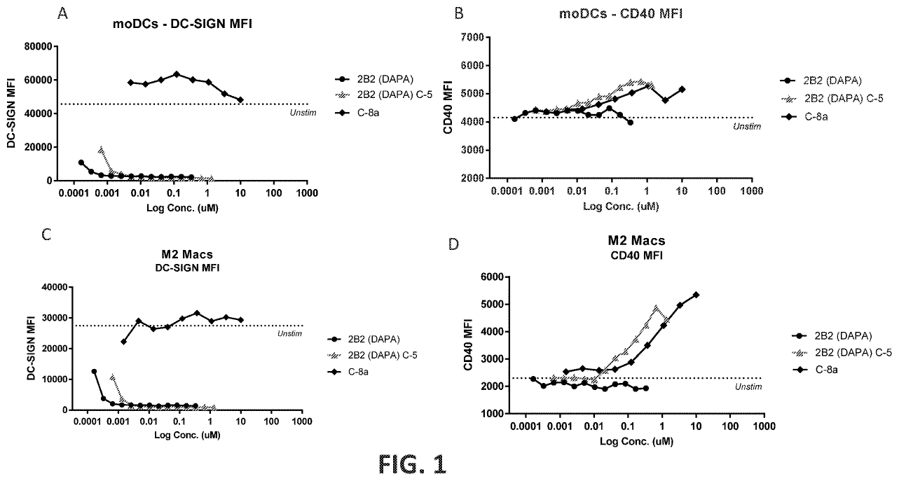

As shown in FIG. 1, 2B2 (DAPA)C-5 induced downregulation of DC-SIGN on monocyte dendritic cells and macrophages (FIGS. 1A and 1C), indicating target engagement. 2B2 (DAPA) C-5 ISACs induced monocyte dendritic cell and macrophage activation as measured by CD86 upregulation (FIGS. 1B and 1D).

### Example 76

### 2B2 (DAPA)C-5 Conjugate Induces DC Activation and Cytokine Secretion in Tg+ Mice

Transgenic mice expressing human DC-SIGN gene (Tg+) or transgene-negative littermate control (Tg−) mice were treated with 2B2 (DAPA)C-5 or isotype control (DAPA)C-5 at 1 milligram per kilogram body weight (mpk) intravenously (i.v.). Mice were bled 6 hours after dosing to collect plasma for analysis of circulating cytokine levels. Spleens were harvested 24 hours post dose and analyzed by flow cytometry to look at CD11c+ dendritic cells.

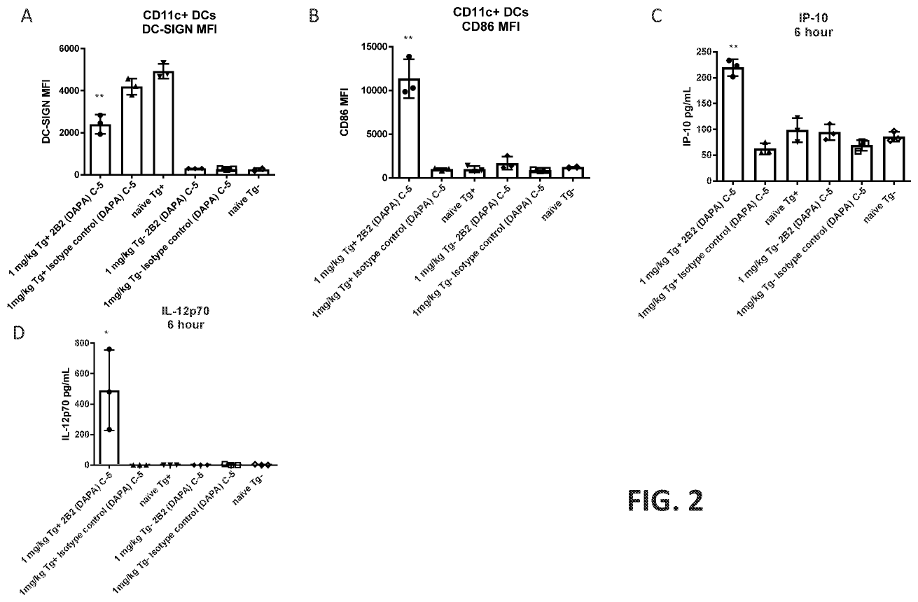

As shown in FIG. 2, Tg+ mice treated with 2B2 (DAPA)C-5 had a significant downregulation of surface DC-SIGN (FIG. 2A), indicating target engagement. Tg+ mice treated with 2B2 (DAPA)C-5 had a significant upregulation of CD86 on the surface of dendritic cells indicating activation (FIG. 2B). Plasma IL-12p70 (FIG. 2D) and IP-10 (FIG. 2C) were significantly increased in Tg+ mice treated with 2B2 (DAPA)C-5 at 6 hours post dose, indicative of on-target activation through DC-SIGN.

### Example 77

### 2B2 (DAPA)C-5 Conjugate Induces DC Activation in MC38 Tumor Cells

Female transgenic mice expressing human DC-SIGN gene (Tg+) or Tg− animals were implanted with 2.5×10⁵ MC38 tumor cells subcutaneously in the hind flank. When tumors reached 100-200 cubic millimeters (mm³), mice were treated with a single dose of 2B2 (DAPA) C-5 or saline as a control. Spleens and tumors were analyzed 24 hours post dose by flow cytometry for DC activation.

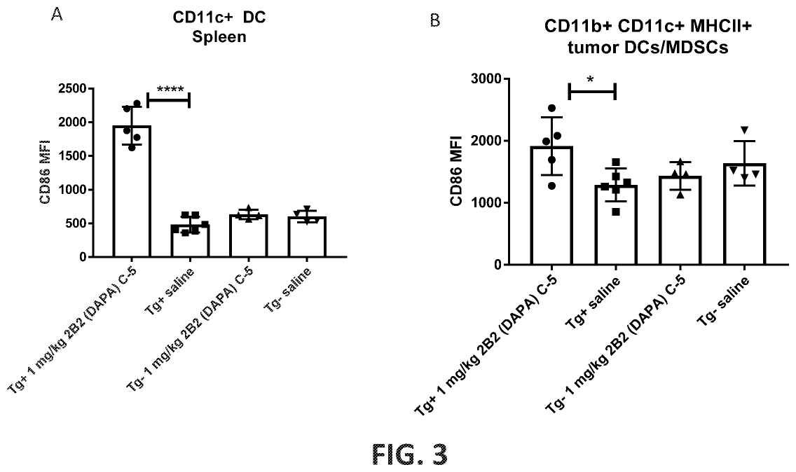

As shown in FIG. 3, Tg+ mice treated with 1 mg/kg of 2B2 (DAPA)C-5 had a significant upregulation of CD86 on the surface of DCs in the spleen (FIG. 3A) and CD11b+ CD11c+ MHCII+ cells in the tumor (FIG. 3B) (a mixed population consisting of dendritic cells, myeloid derived suppressor cells (MDSCs) and other antigen presenting cells).

### Example 78

### 2B2 (DAPA)C-5 Conjugate does not Inhibit Tumor Growth in MC38 Tumor Model

Female transgenic mice expressing human DC-SIGN gene (Tg+) or Tg− animals were implanted with 2.5×10⁵ MC38 tumor cells subcutaneously in the hind flank. Tumors were measured 3 times a week throughout the course of the study. When tumors reached 100-200 cubic millimeters (mm³), mice were treated with a single dose of 2B2 (DAPA)C-5 or saline as a control. No difference in tumor size was observed in Tg+ mice dosed with 1 mg/kg of 2B2 (DAPA)C-5.

### Example 79

### RIG-1 Hairpins are Active on DC-SIGN Expressing Monocyte Derived DCs (moDCs)

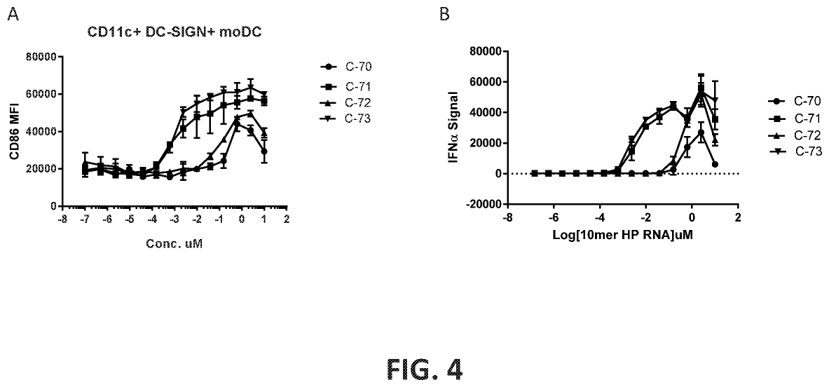

Primary human monocytes were isolated from a leukapheresis using magnetic bead selection and frozen for storage in liquid nitrogen. For monocyte DC (moDC) differentiation, cells were thawed and incubated in media containing GM-CSF and IL-4 for 7 days. After the differentiation process, media was washed off and replaced with fresh media containing indicated compounds in dose response. 24 hours after incubation with indicated compounds, cells were evaluated by flow cytometry for activation and supernatant was collected for IFN alpha analysis. C-70, C-71, C-72 and C-73 all induced monocyte dendritic cell activation as measured by CD86 upregulation (FIG. 4A) and interferon alpha secretion (FIG. 4B) by moDC in a dose dependent manner.

### Materials and Methods Used in Examples

### Mouse Tumor Experiments and Drug Antibody Conjugate Treatment.

MC38 cells were grown in 10% Dulbecco's Modified Eagle Medium (DMEM) at 80% confluence prior to implant. Cells growing in log phase were harvested and washed with Hank's Balanced Salt Solution (HBSS) prior to implant. 100 ul of 2.5×10e6 MC38 cells were implants subcutaneously in the hind flank of mice, using insulin syringes, gauge 31. Mice were anesthetized with isoflurane, shaved prior to implant and measured for body weight. Starting at day 5-7 post implant mice were measured using digital calipers using the formula V=(W(2)×L)/2 to determine tumor volume in mm3 (W=tumor width, L=tumor length). Mice were measured every other day and monitored for signs of distress, body weight loss and possible ulcerations. Compounds were administered intravenously when tumors were between 100-200 mm³ using a 1 ml syringe with a 27% gauge needle. Retro-orbital intravenous injection of ISAC (200 μl) and/or checkpoint blockade was administered under anesthesia. Unless otherwise stated, drug-antibody conjugate dosing was once and checkpoint blockade was 2-3 times throughout the study with 3-4 days in between doses. where indicated, blood was collected at 6 and 24 hours post dose. Mice were sacrificed at indicated time points post dose and tumors, spleen and lymph nodes were harvested where indicated for analysis

### Tumor and Spleen Processing and Flow Cytometry Protocol:

Tumors and/or spleens were extracted at the timepoints indicated from animals. Spleens were processed into a single cell suspension using glass slides and passed through a 100 micron mesh filter. Spleens were lysed in 1 mL of ACK lysis buffer (Life Technologies) for 5 minutes at room temperature. After lysis, cells were pelleted and resuspended in complete RMPI medium (RPMI Media 1640 with 10 percent fetal bovine serum (FBS), 0.05 mM 2-mercaptoethanol, 1 percent Penicillin-Streptomycin-Glutamine, 1 percent non-essential amino acids, 1 percent HEPES, 1 percent sodium pyruvate (all media reagents from Thermo Fisher). Tumors were extracted and put into digestion media in gentleMACS C tubes (Miltenyi Biotec). Digestion media consists of Dulbecco's Modified Eagle Medium with 0.04 U/mL Dispase (StemCell Technologies), 0.1 mg/mL Collagenase P (Sigma) and 0.1 mg/mL DNase (Sigma). Tumors were incubated with in digestion media and then processed using the gentleMACS Dissociator (Miltenyi Biotec Inc, San Diego, Calif.) to obtain a single cell suspension. After processing, cells were filtered in 100 uM filters (Miltenyi Biotec Inc).

1-2 million cells for each sample were then stained with a cocktail of antibodies to determine impact of the treatments on dendritic cells, myeloid cells and T cells. For FACS analysis, cells were stained with a fixable, amine reactive dye to label dead cells (Zombie UV™ fixable viability kit, Biolegend) in PBS. For antibody staining, indicated antibodies (see table below) were diluted in PBS with 0.5% Bovine serum albumin (BSA, from Sigma). Samples were incubated at 4° C. for 30 minutes and then washed 2 times with PBS with 0.5% BSA. Cells were fixed with stabilizing fixative (BD). After staining, cells were evaluated on the BD LSRFortessa™ cell analyzer (BD Biosciences, San Jose, Calif.).

Dendritic cells were identified as MHCII high CD11c high cells and further gated on expression of CD8 and CD11 b to identify CD8+ DC subsets and CD11b+ DCs where noted. Monocytic myeloid derived suppressor cells were identified as CD45+ cells in tumors that express CD11b, MHCII, F4/80, Ly6C and are intermediate for Ly6G.

TABLE 21
FACS antibodies

| | | | Species |
|---|---|---|---|
| Marker | Clone | Vendor | Reactvity |
| CD45 | 30F11 | BD | Mouse |
| CD8 | 53-6.7 | BD | Mouse |
| Ly6G | 1A8 | Biolegend | Mouse |
| CD11b | M1/70 | Biolegend | Mouse |
| CD11c | N418 | Biolegend | Mouse |
| CD86 | GL-1 | Biolegend | Mouse |
| PDL1 | 10F.9G2 | Biolegend | Mouse |
| Ly6C | HK1.4 | Biolegend | Mouse |
| MHCII | M5.114 | Biolegend | Mouse |
| F4/80 | BM8 | Biolegend | Mouse |
| CD3 | 17A2 | Biolegend | Mouse |
| HLA-DR | L243 | Biolegend | Human |
| CD86 | IT2.2 | Biolegend | Human |
| CD11c | 3.9 | Biolegend | Human |
| DC-SIGN | 9E9A8 | Biolegend | Human |

### Monocyte Isolation

Peripheral blood Leukopaks from normal human donors were obtained from HemaCare. Leukopaks were aliqouted into 50 mL conical tubes (BD Falcon) and centrifuged at 300 g to 30 minutes to pellet cells. Cells were resuspended in Phosphate Buffered Saline (PBS) containing 2% FBS and 1 mM EDTA to a final concentration of 10⁸ per mL. EasySep Human CD14 Positive Selection Cocktail (StemCell Technologies) was added at 100 μL per mL of cells. CD14+ cells were obtained by positive magnetic selection by following manufactures recommended protocol. Following selection cells were pelleted by centrifugation at 300 g for 10 minutes and resuspended in Recovery™ Cell Culture freezing medium (Thermo Fisher) at 50-100 million cells per mL in cryovials. Cells were frozen in −80 degree C. freezer for at least one day and transferred to liquid nitrogen for storage. Cells were kept in liquid nitrogen until use.

moDC and M2 Macrophage Differentiation

Human CD14+ monocytes were isolated and frozen as described. On the day of differentiation, previously collected and frozen CD14+ monocytes were thawed in a 37 degree C. water bath until just thawed and added immediately to prewarmed complete RPMI medium (cRPMI). Cells were then spun at 1500 rotations per minute (rpm) for 5 minutes in a table top centrifuge to pellet cells. Medium was removed and cells were resuspended in fresh, prewarmed cRPMI medium. Cells were counted and plated at 40,000-80,000 cells per well in a 384 well flat bottom tissue culture plate (Greiner).

For monocyte dendritic cell (moDC) differentiation, cells were cultured in 40 μL final volume with 53 ng/mL of recombinant human GM-CSF (R4D Systems) and 20 ng/mL recombinant human IL-4 (R&D Systems) for 7 days. Cells were washed and fresh, cRPMI was added prior to stimulation with compounds or antibody drug conjugates.

For M2 macrophage differentiation, cells were cultured in 40 μL final volume with a final concentration of 100 ng/mL of recombinant human MCSF. 6 days after differentiation, 20 ng/mL of IL-4 was added to polarize macrophages to an M2 phenotype. 24 hours after polarization, cells were washed and fresh, cRPMI was added prior to stimulation with compounds or antibody drug conjugates.

24 hours after activation with compounds, cells were evaluated by flow cytometry according to the described protocol using antibody clones described in flow cytometry protocol section. DC-SIGN+ CD11c+ HLA-DR+ cells were identified and assessed for CD86 expression and levels of DC-SIGN.

### IP-10 ELISA

Plasma was collected at indicated timepoints and analyzed with a Mouse IP-10 Platinum ELISA kit (eBioscience Affymetrix). Plasma was diluted 1:100 and the protocol was followed according to the manufacturer's recommendations. Data was collected using an Enspire spectro-photometer using 450 nM as the primary wavelength.

### MesoScale Discovery Cytokine Analysis (MSD)

Plasma was collected at indicated timepoints and analyzed with a mouse Proinflammatory Panel 1 (mouse) Kit V-PLEX™ 10 plex from MesoScale Discovery. 25 μL of plasma per sample was used and protocol was followed according to the manufacturer's recommendations. Data were collected and analyzed using a Sector Imager 6000.

### INFα2b Alphalisa

Supernatants were collected 18-24 hours after incubation of human monocyte derived DCs with indicated compounds and analyzed using a human Interferon alpha 2b AlphaLISA (Perkin Elmer). 10 μL of supernatant was added to AlphaProxiPlate-384 well plates (Perkin Elmer). The assay was run according to the manufacturer's recommendations and results were read using an Enspire spectro-photometer.

### Mouse Info and Breeding

Human DC-SIGN transgenic mice (Tg+) (Schaefer et al., J. Immunol. (2008) 180 (10) 6836-6845) were bred to Signr1 deficient mice (−/− or KO) (Orr et al., Glycobiology (2013) 23(3): 363-380). Human DC-SIGN expression was checked using PCR to genotype the mice. Human DC-SIGN Tg+ Signr1 −/− mice or human DC-SIGN Tg− Signr1 −/− mice were tested with compounds as indicated in the above examples.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. An antibody or antigen binding fragment thereof that binds to human DC-SIGN protein, wherein the antibody or antigen binding fragment thereof has a higher affinity to human DC-SIGN than human L-SIGN.

2-17. (canceled)

18. A conjugate comprising an anti-DC-SIGN antibody or an antigen binding fragment thereof, coupled to drug moiety (D), wherein D is optionally coupled via a linker (L), wherein the linker optionally comprises one or more cleavage elements.

19. The conjugate of claim 18 comprising Formula (III):
Ab-(L-(D)ₘ)ₙ (Formula (III))
wherein:
Ab is the anti-DC-SIGN antibody or a functional fragment thereof;
L is a linker comprising one or more cleavage or non-cleavable elements;
D is the drug moiety;
m is an integer from 1 to 8; and
n is an integer from 1 to 20.

20. The conjugate of claim 19, wherein the drug moiety is an immunostimulatory molecule, a cytotoxic molecule, a radionuclide, etc.

21. (canceled)

22. The conjugate of claim 20, where in the immunostimulatory molecule is a dendritic cell stimulating compound, for example, a DEC-205 agonist, FLT3 ligand, granulocyte macrophage colony-stimulating factor (GM-CSF), an agonist of a Toll-like receptor (TLR) (e.g., TLR1, TLR2, TLR3, TLR4, TLR5, TLR6, TLR7, TLR8, TLR9, TLR10), RIG-I, MDA-5, LGP2, a C-type lectin receptor agonist, NOD1, NOD2, costimulatory compounds such as IL-15 or agonists of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

23. The conjugate of claim 22, wherein the immunostimulatory molecule is an agonist of TLR7.

24. The conjugate of claim 22, wherein the immunostimulatory molecule is an agonist of RIG-I.

25. The conjugate of claim 23 comprising Formula (II), or pharmaceutically acceptable salt thereof: 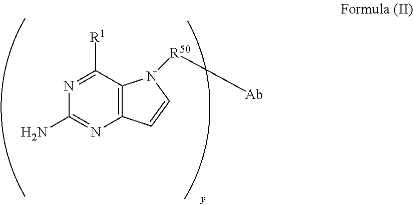
wherein:
R⁵⁰ is 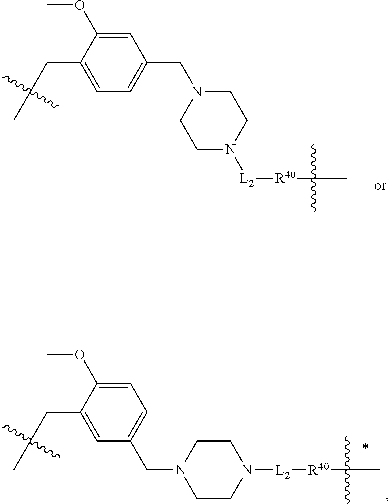
where the * indicates the point of attachment to Ab;
R¹ is —NHR² or —NHCHR²R³;
R² is —C₃-C₆alkyl or —C₄-C₆alkyl;
R³ is LiOH;
L₁ is —(CH₂)ₘ—;
L₂ is —(CH₂)ₙ—, —((CH₂)ₙO)ₜ(CH₂)ₙ—, —(CH₂)ₙX₁(CH₂)ₙ—, —(CH₂)ₙNHC(═O)(CH₂)ₙ—, —(CH₂)ₙNHC(═O)(CH₂)ₙC(═O)NH(CH₂)ₙ—, —((CH₂)ₙO)ₜ(CH₂)ₙNHC(═O)(CH₂)ₙ, —C(═O)(CH₂)ₙ—, —C(═O)((CH₂)ₙO)ₜ(CH₂)ₙ—, —C(═O)((CH₂)ₙO)ₜ(CH₂)ₙX₁(CH₂)ₙ—, —C(═O)((CH₂)ₙO)ₜ(CH₂)ₙNHC(═O)(CH₂)ₙ—, —C(═O)((CH₂)ₙO)ₜ(CH₂)ₙC(═O)NH(CH₂)ₙ—, —C(═O)NH((CH₂)ₙO)ₜ(CH₂)ₙX₁(CH₂)ₙ—, —C(═O)X₂X₃C(═O)((CH₂)ₙO)ₜ(CH₂)ₙ—, —C(═O)X₂X₃C(═O)(CH₂)ₙ—, —C(═O)X₂C(═O)(CH₂)ₙNHC(═O)(CH₂)ₙ—, —C(═O)X₂C(═O)(CH₂)ₙNHC(═O)((CH₂)ₙO)ₜ(CH₂)ₙ—, —C(═O)(CH₂)ₙC(R⁷)₂—, —C(═O)(CH₂)ₙC(R⁷)₂SS(CH₂)ₙNHC(═O)(CH₂)ₙ—, —(CH₂)ₙX₂C(═O)(CH₂)ₙNHC(═O)((CH₂)ₙO)ₜ(CH₂)ₙ— or —C(═O)(CH₂)ₙC(═O)NH(CH₂)ₙ;
R⁴⁰ is 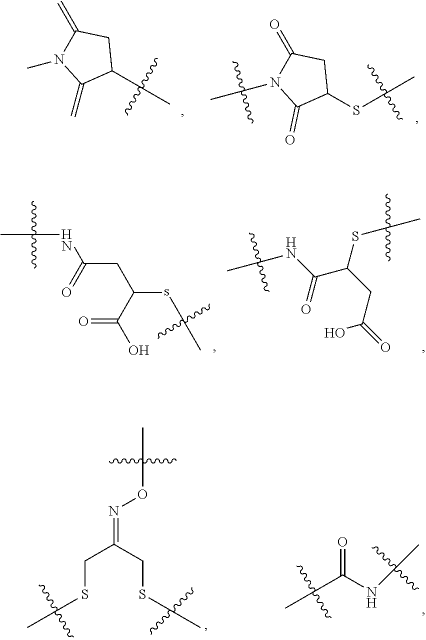
—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—, 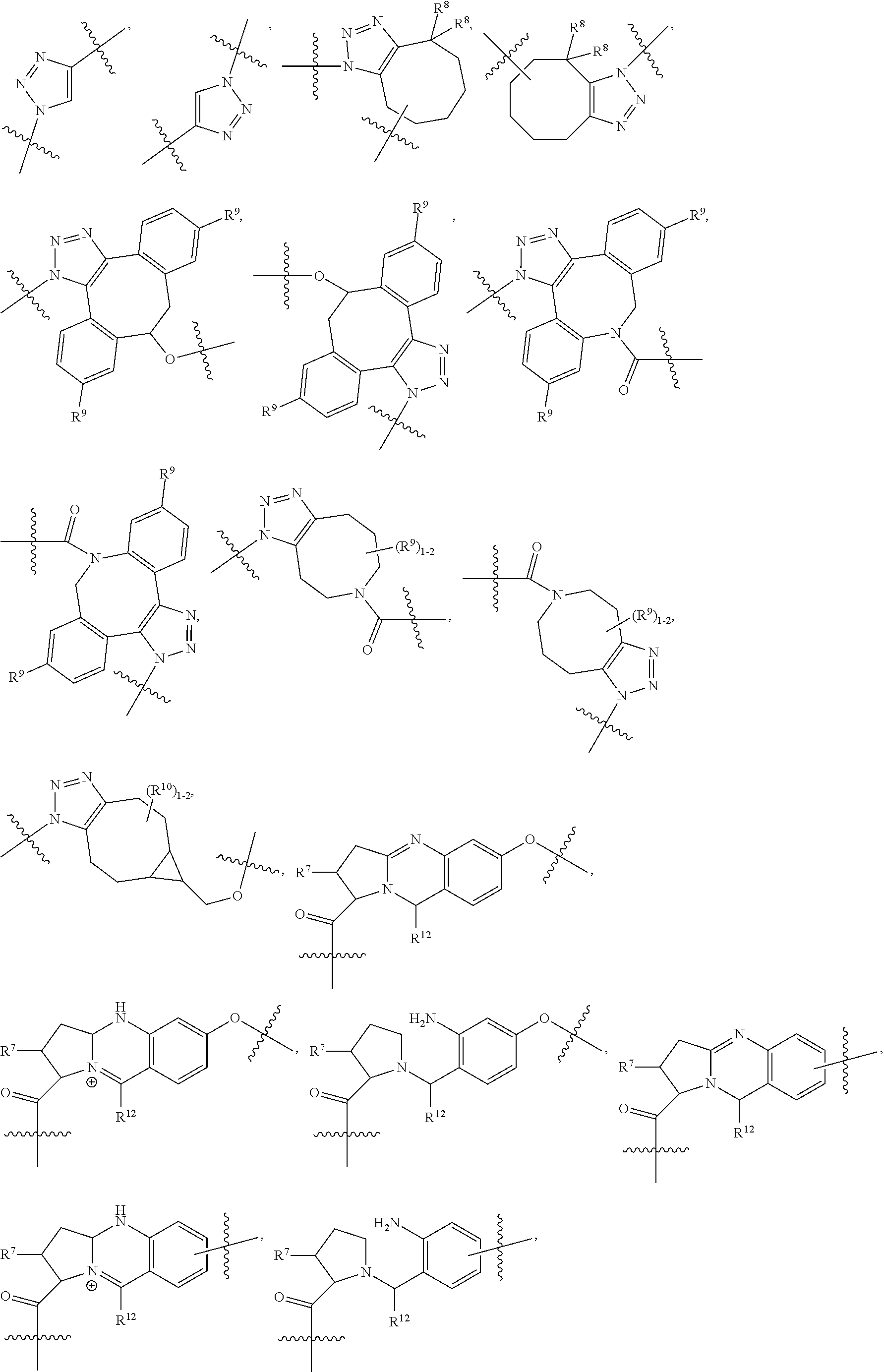 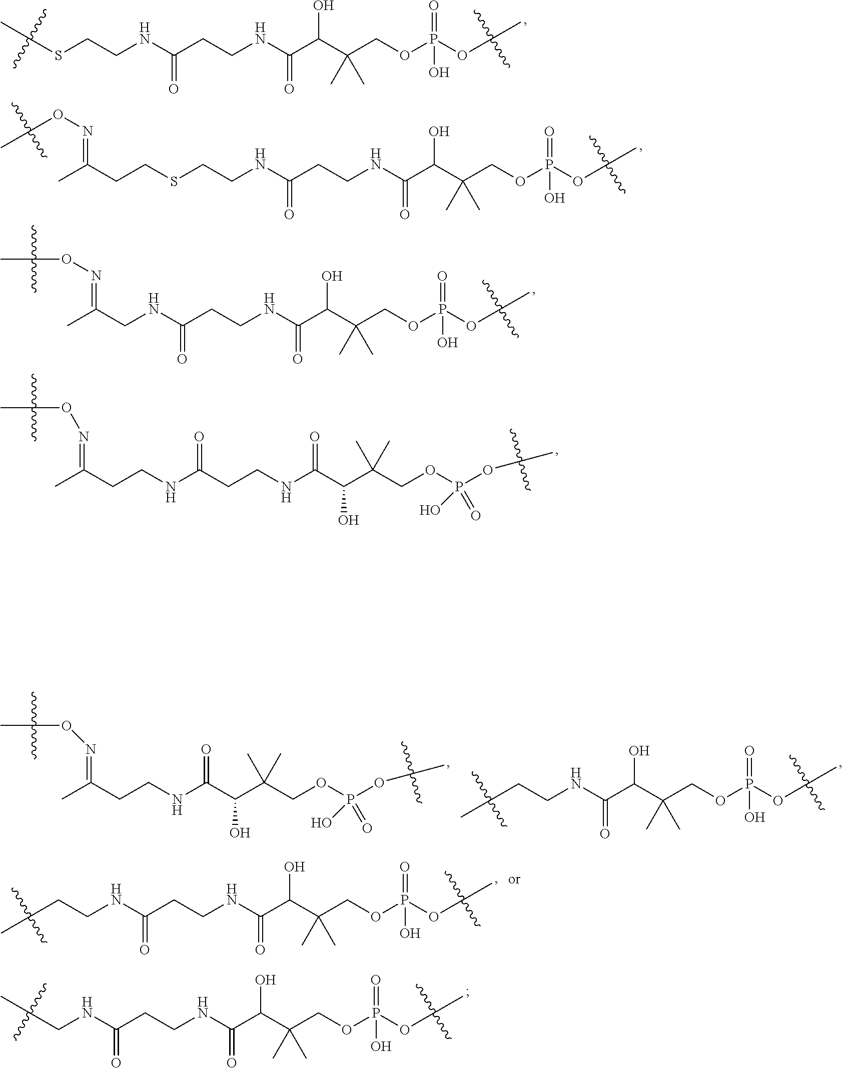
X₁ is 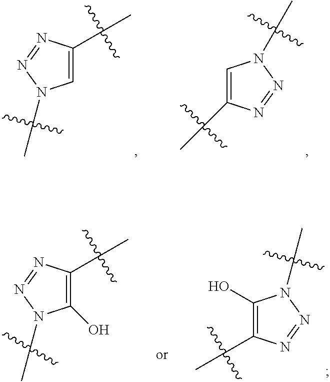 [SMILES: *c1cn(*)nn1 ; *c1cn(*)nn1 ; *c1nnn(*)c1O ; *c1nnn(*)c1O]
X₂ is 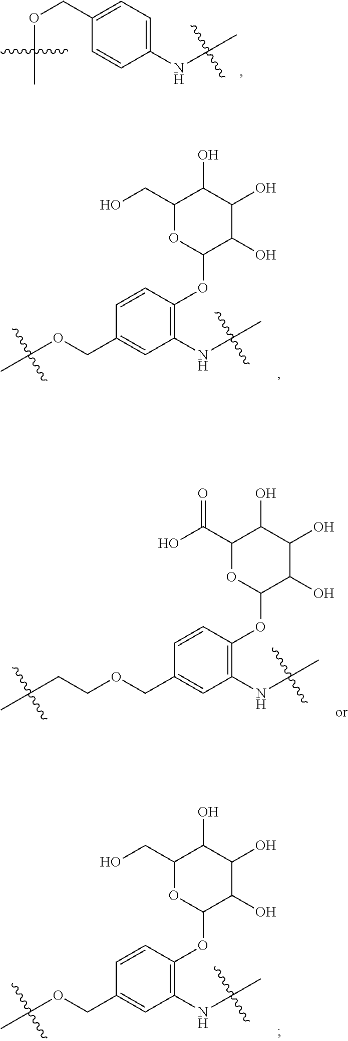 [SMILES: *Nc1ccc(CO*)cc1 ; *Nc1cc(CO*)ccc1OC1OC(CO)C(O)C(O)C1O ; *CCOCc1ccc(OC2OC(C(=O)O)C(O)C(O)C2O)c(N*)c1 ; *Nc1cc(CO*)ccc1OC1OC(CO)C(O)C(O)C1O]
X₃ is 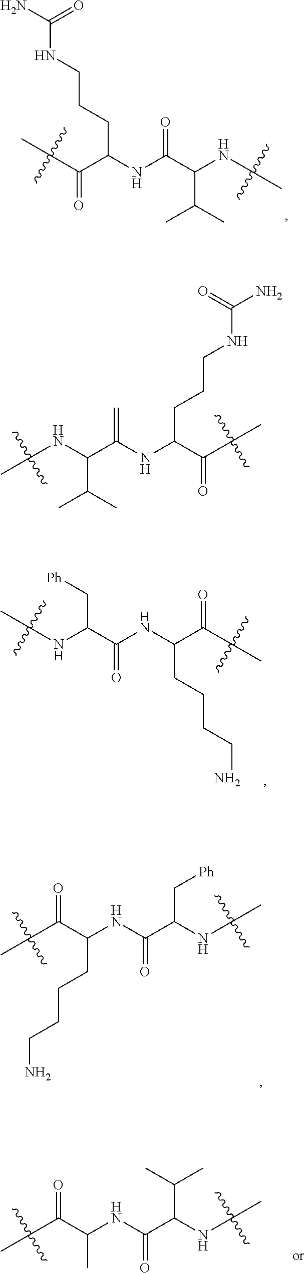 [SMILES: *NC(C(=O)NC(CCCNC(N)=O)C(*)=O)C(C)C ; *NC(C(=C)NC(CCCNC(N)=O)C(*)=O)C(C)C ; *NC(Cc1ccccc1)C(=O)NC(CCCCN)C(*)=O ; *NC(Cc1ccccc1)C(=O)NC(CCCCN)C(*)=O ; *NC(C(=O)NC(C)C(*)=O)C(C)C] 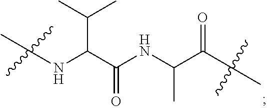 [SMILES: *NC(C(=O)NC(C)C(*)=O)C(C)C]
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

26. The conjugate of claim 25, wherein the antibody conjugate of Formula (II) comprises the structure of Formula (IIa) or Formula (IIb): 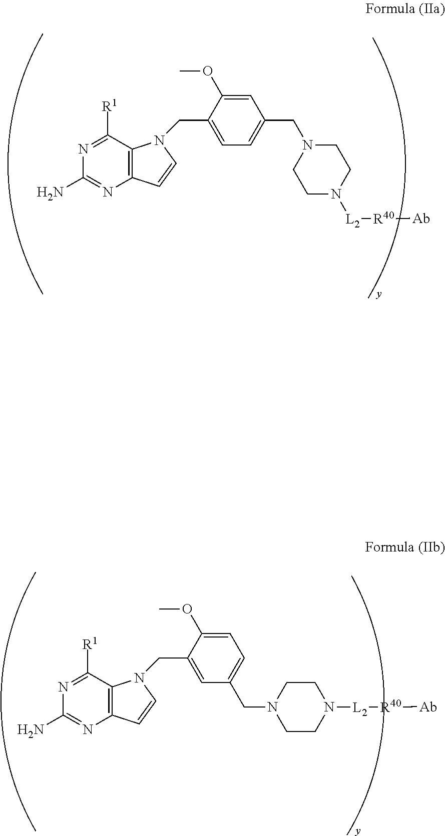
wherein:
R¹ is —NHR²;
R² is —C₄-C₆alkyl;
L₂ is —(CH₂)ₙ—, —((CH₂)ₙO)ₜ(CH₂)ₙ—, —(CH₂)ₙX₁(CH₂)ₙ—, —C(═O)(CH₂)ₙ—, —C(═O)((CH₂)ₙO)ₜ(CH₂)ₙ—, —C(═O)((CH₂)ₙO)ₜ(CH₂)ₙX₁(CH₂)ₙ—, —C(═O)NH((CH₂)ₙO)ₜ(CH₂)ₙX₁(CH₂)ₙ—, —C(═O)X₂X₃C(═O)((CH₂)ₙO)ₜ(CH₂)ₙ— or —C(═O)X₂C(═O)(CH₂)ₙNHC(═O)(CH₂)ₙ—;
R⁴⁰ is 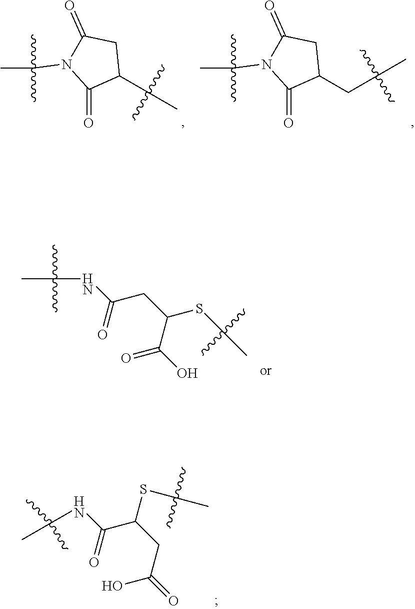 [SMILES: *C1CC(=O)N(*)C1=O ; *CC1CC(=O)N(*)C1=O ; *NC(=O)CC(S*)C(=O)O ; *NC(=O)C(CC(=O)O)S*]
X₁ is 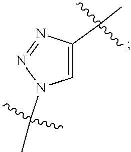 [SMILES: *c1cn(*)nn1]
X₂ is 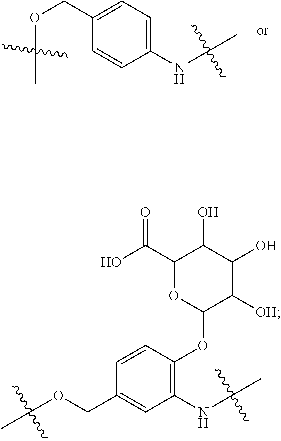 [SMILES: *Nc1ccc(CO*)cc1 ; *Nc1cc(CO*)ccc1OC1OC(C(=O)O)C(O)C(O)C1O]
X₃ is; 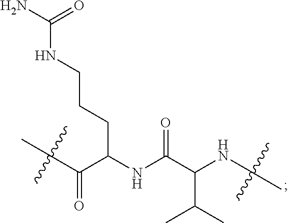 [SMILES: *NC(C(=O)NC(CCCNC(N)=O)C(*)=O)C(C)C]
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

27. The conjugate of claim 26, wherein
R¹ is —NHR²;
R² is —C₄-C₆alkyl;
L₂ is —(CH₂)ₙ— or —C(═O)(CH₂)ₙ;
R⁴⁰ is 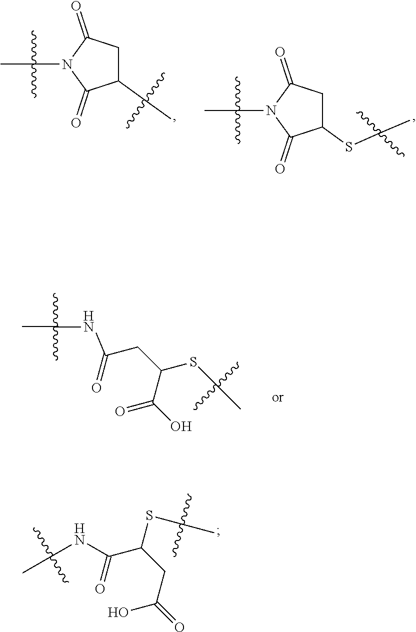 [SMILES: *C1CC(=O)N(*)C1=O ; *SC1CC(=O)N(*)C1=O ; *NC(=O)CC(S*)C(=O)O ; *NC(=O)C(CC(=O)O)S*]
and
each n is independently selected from 1, 2, 3, and 4, and
y is an integer from 1 to 16.

28. The conjugate of claim 25, wherein the conjugate has a hydrophobicity index of 0.8 or greater, as determined by hydrophobic interaction chromatography.

29. The conjugate of claim 23 comprising any of the following formulas: 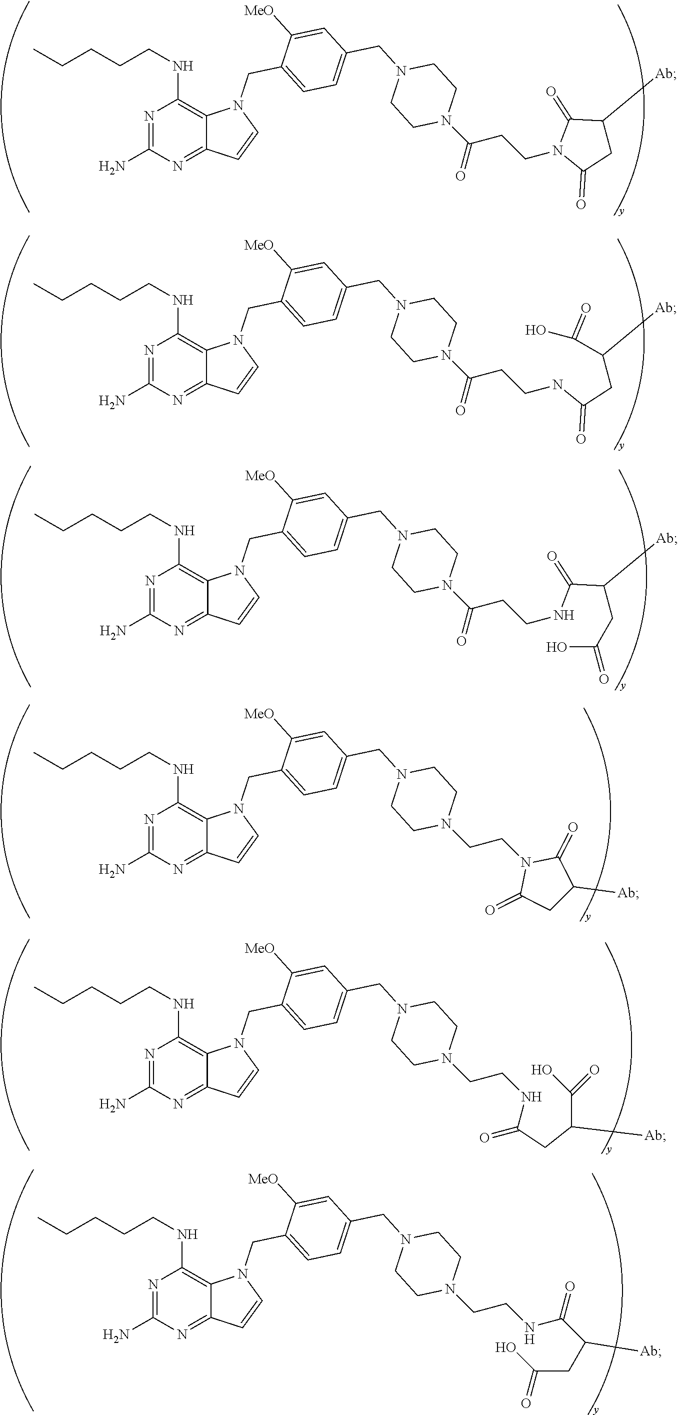 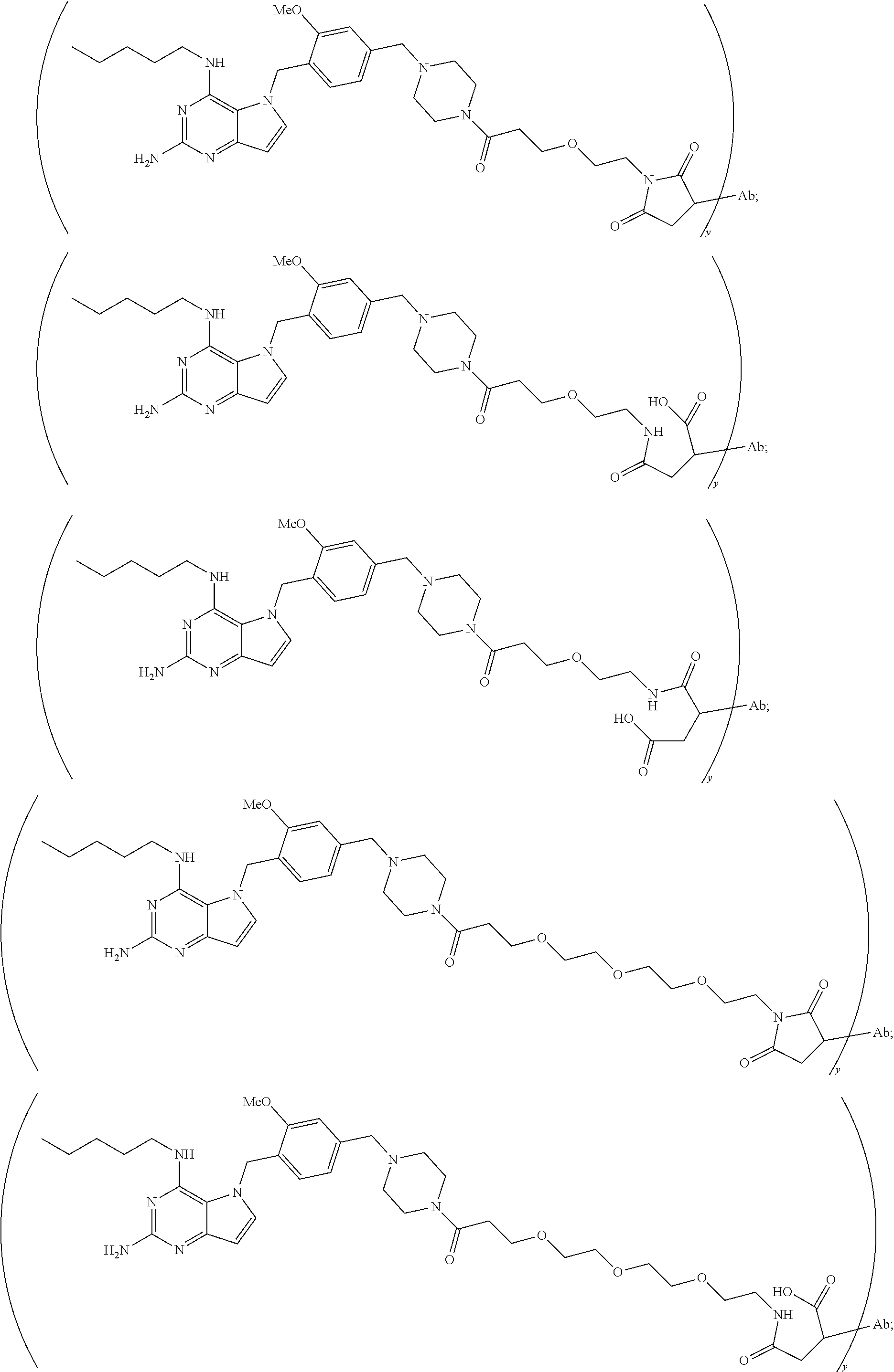 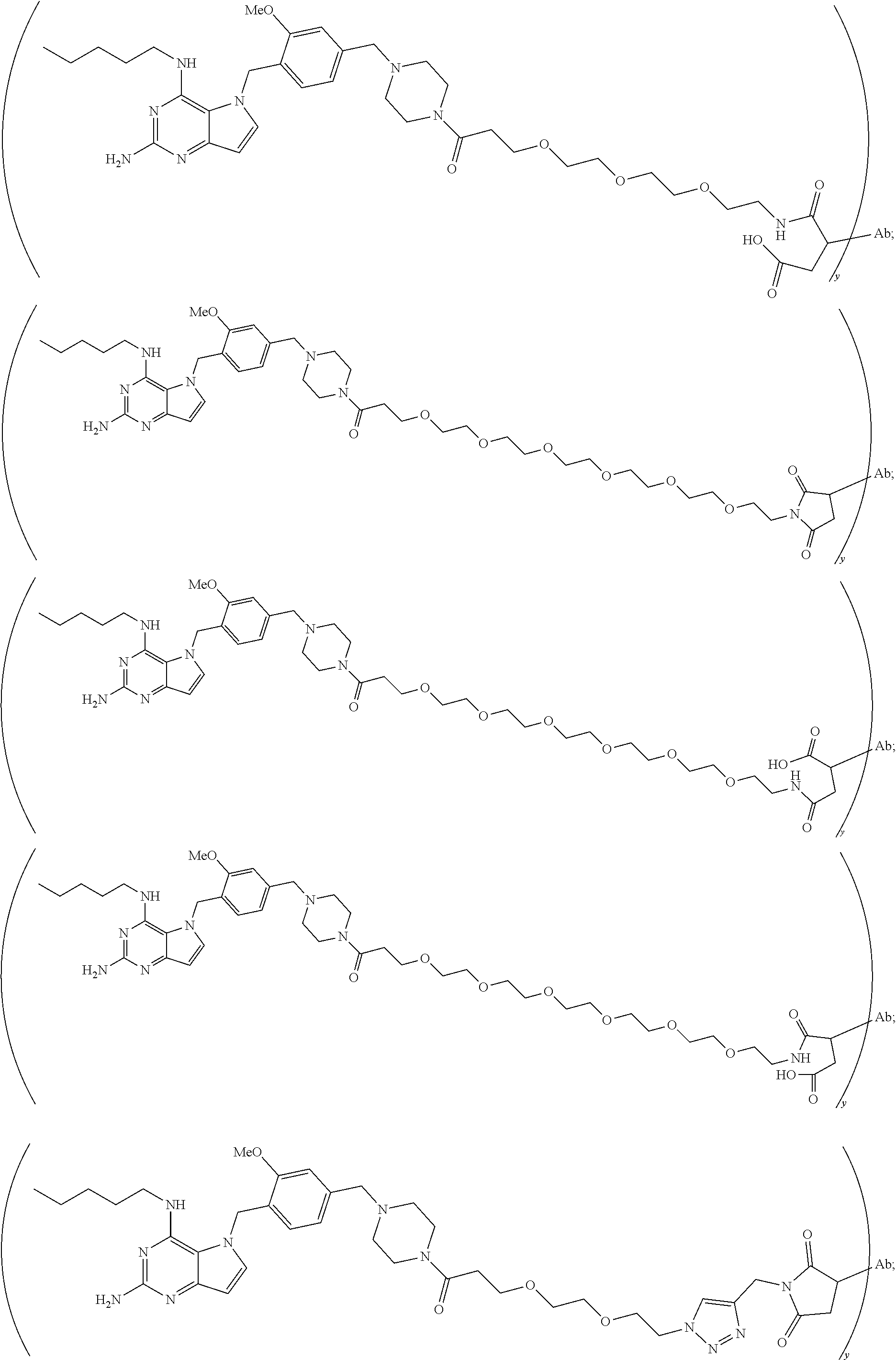 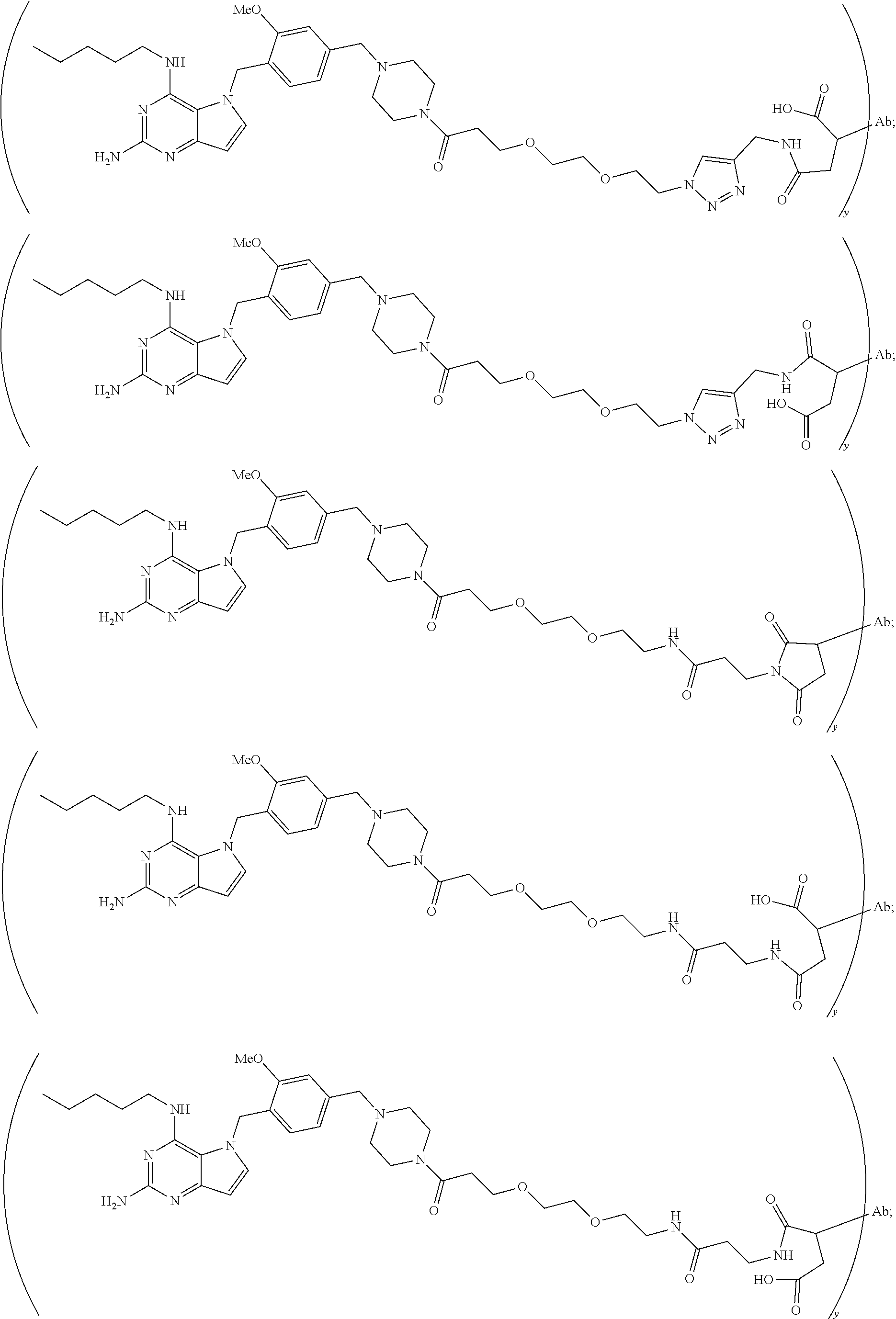 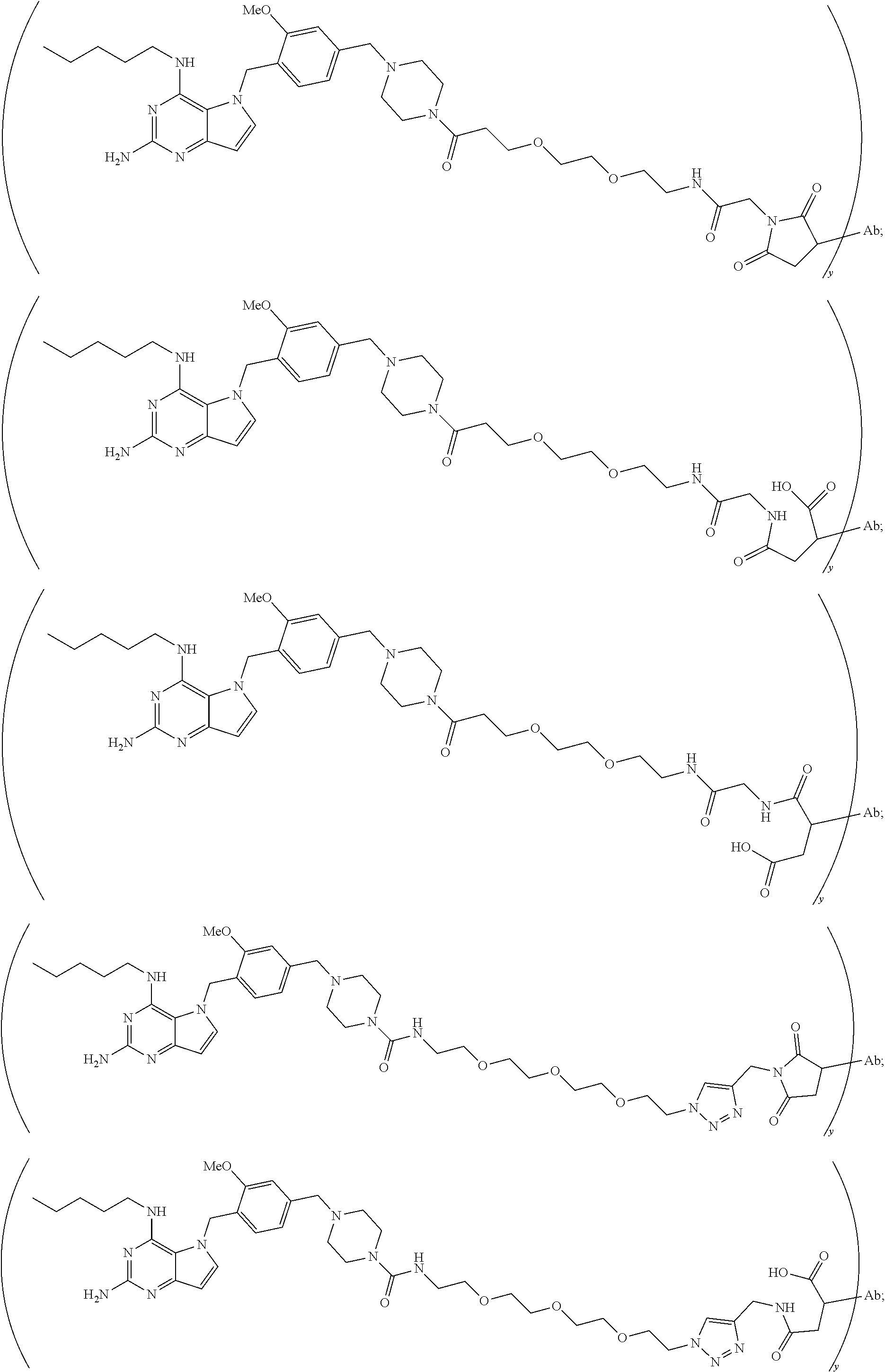 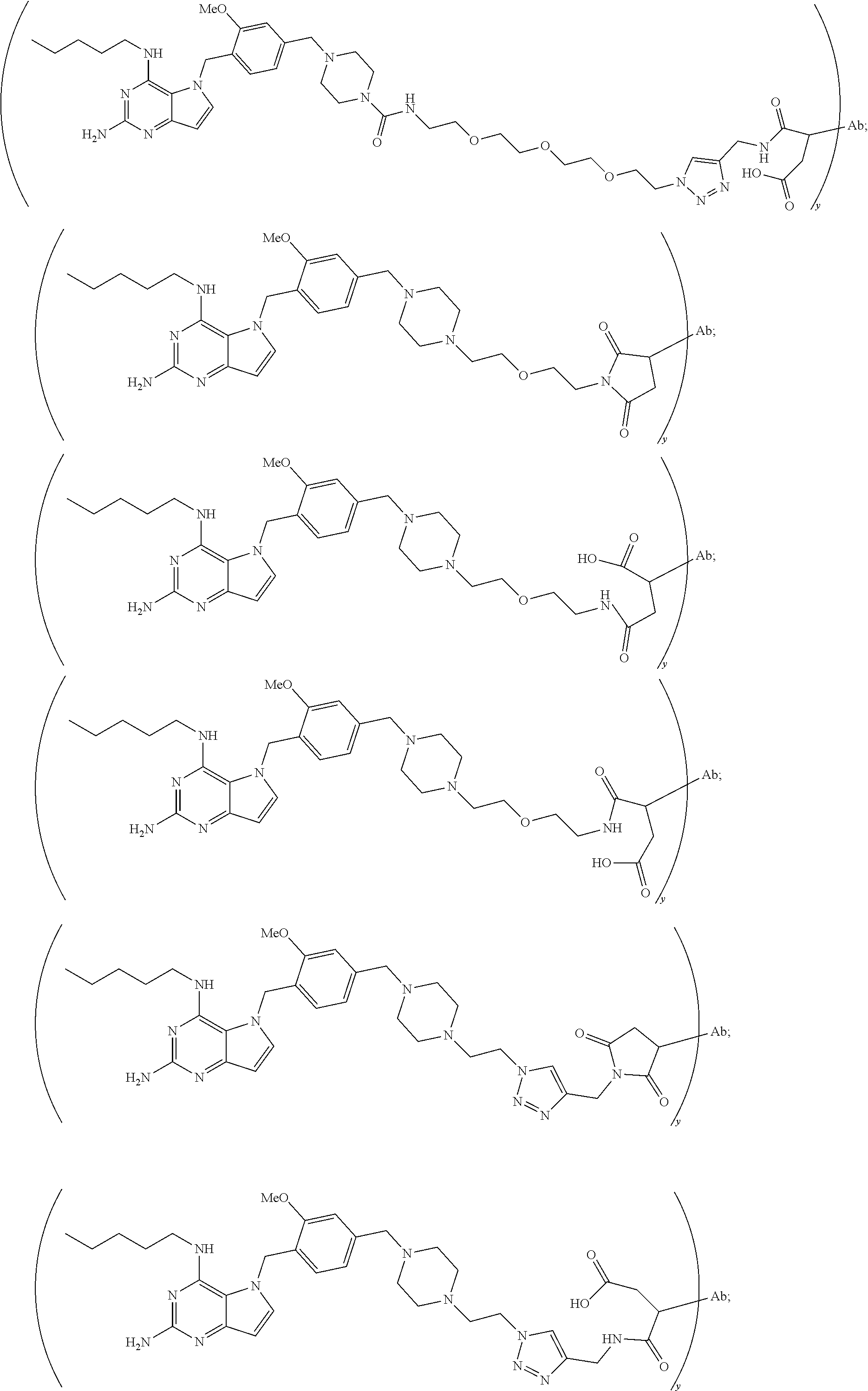 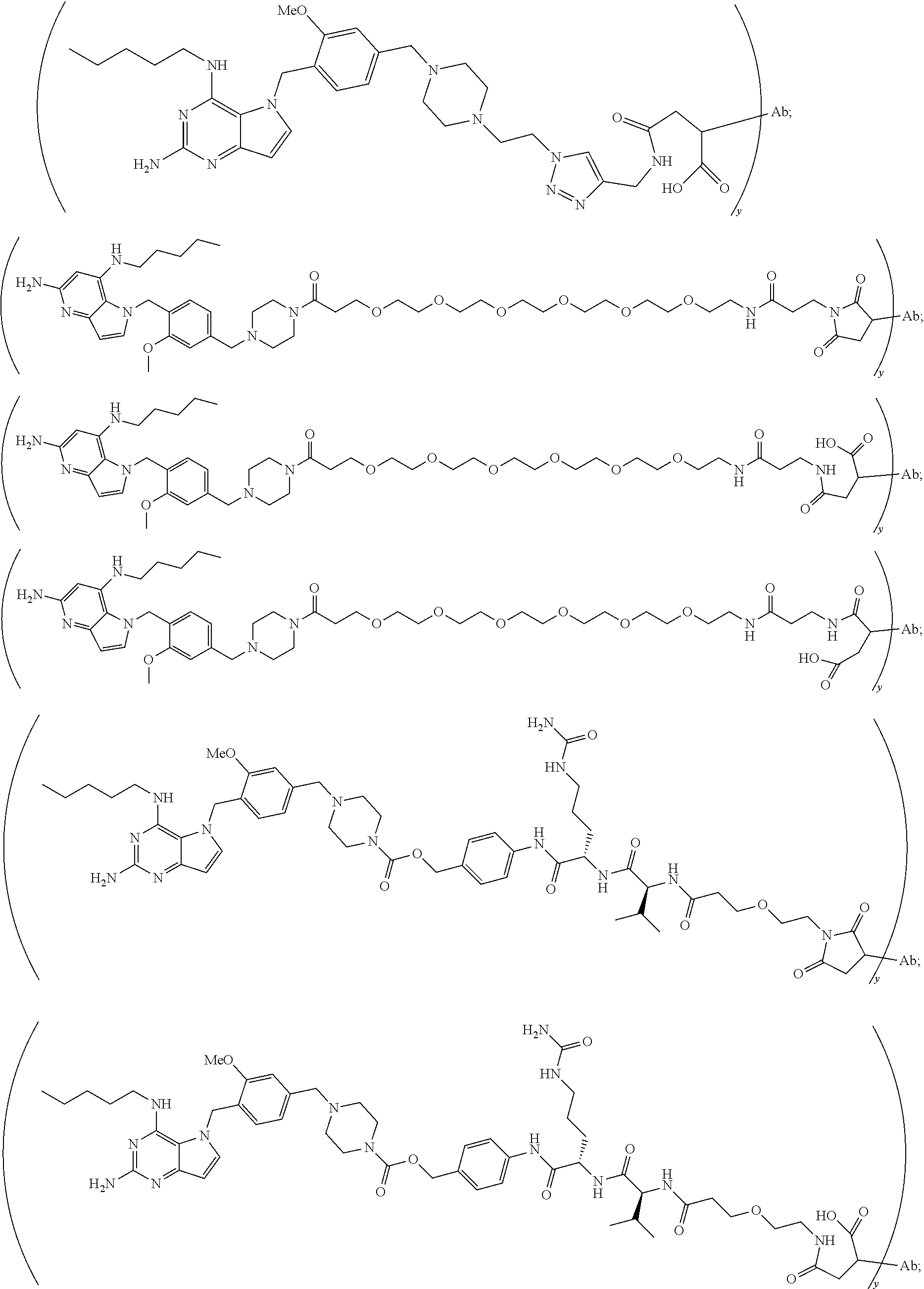 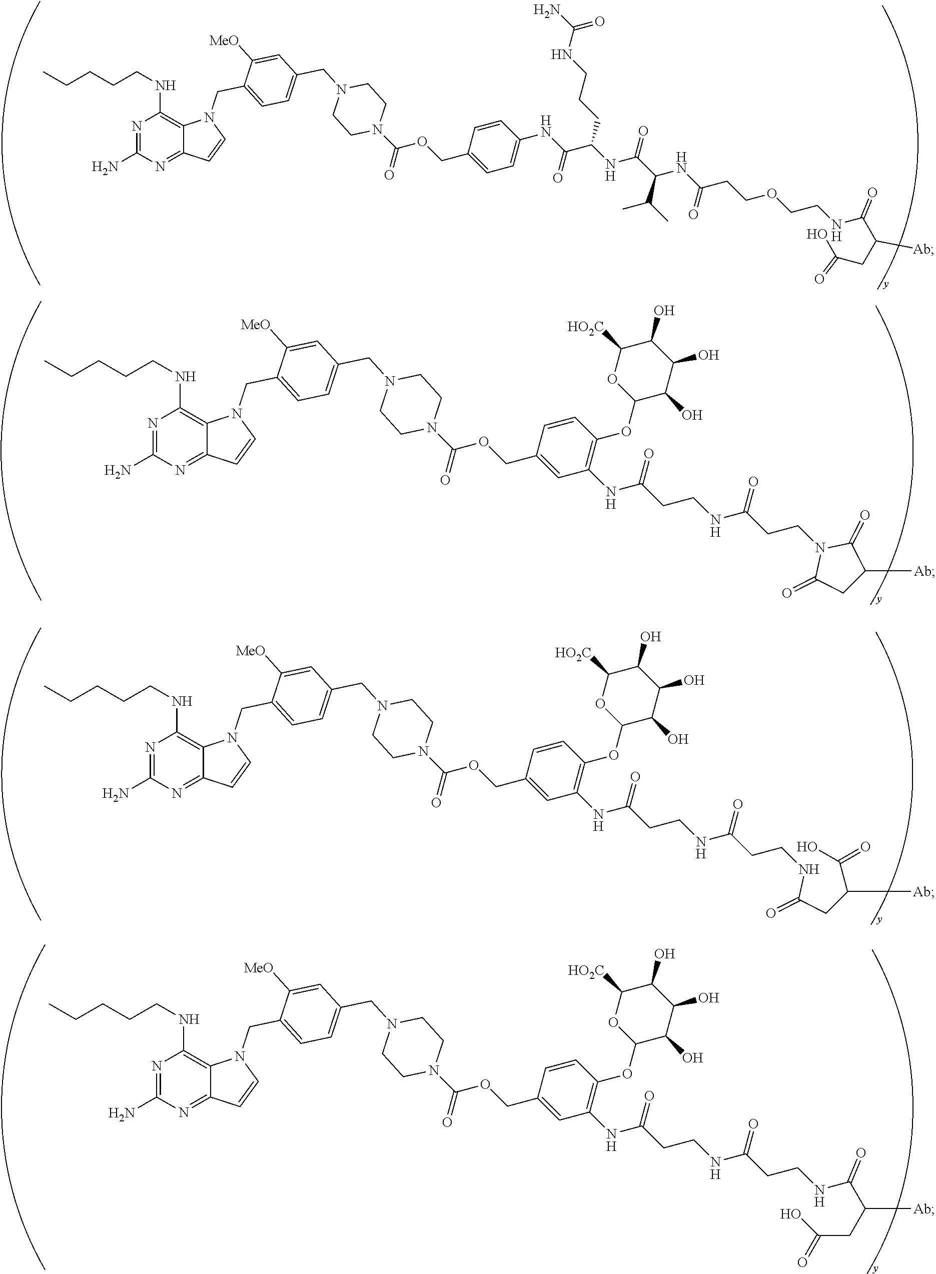 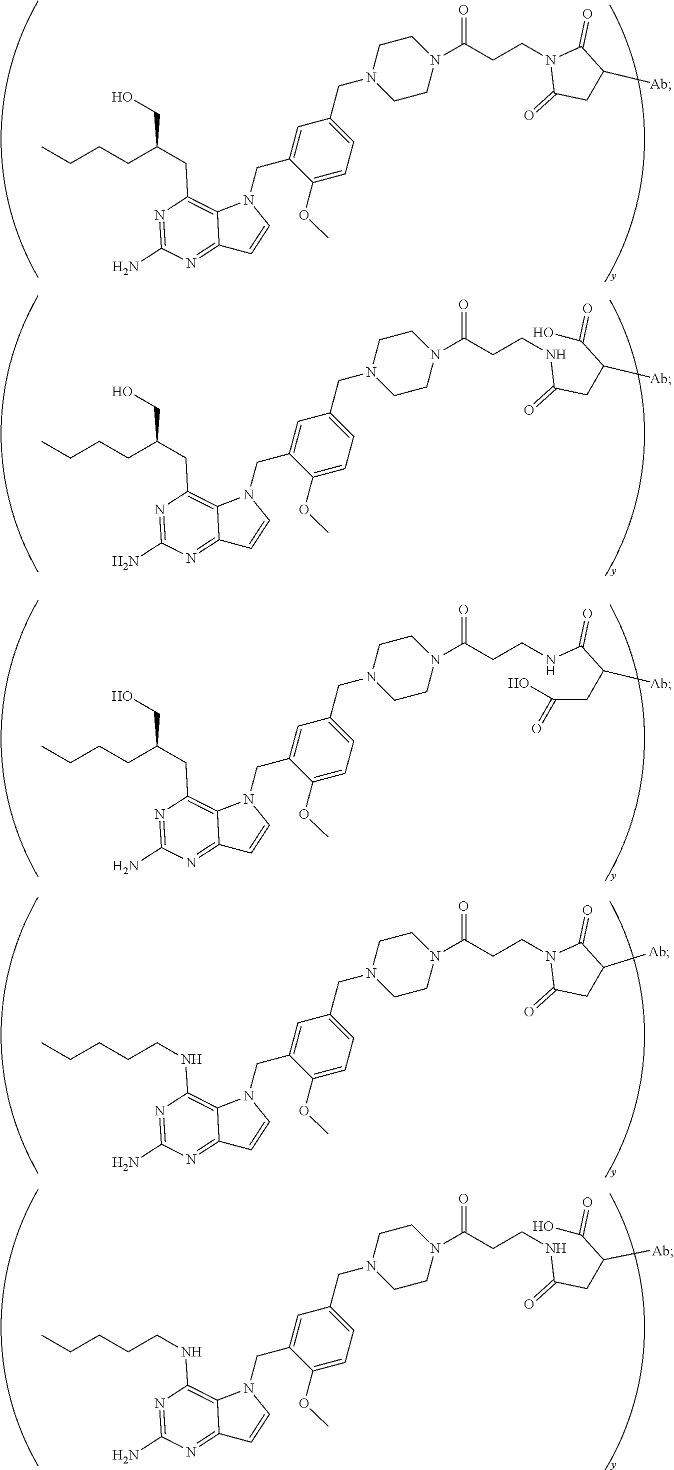 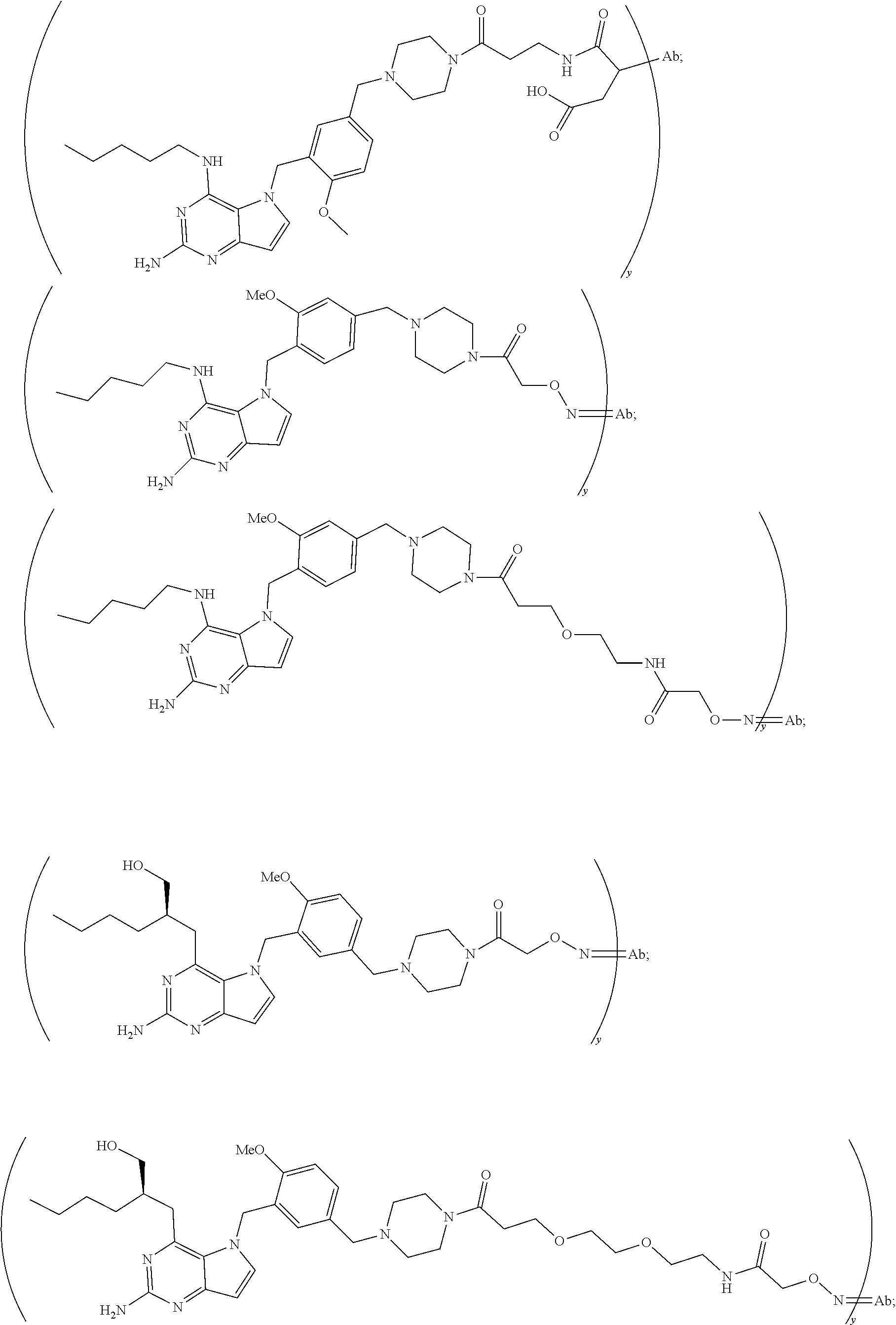 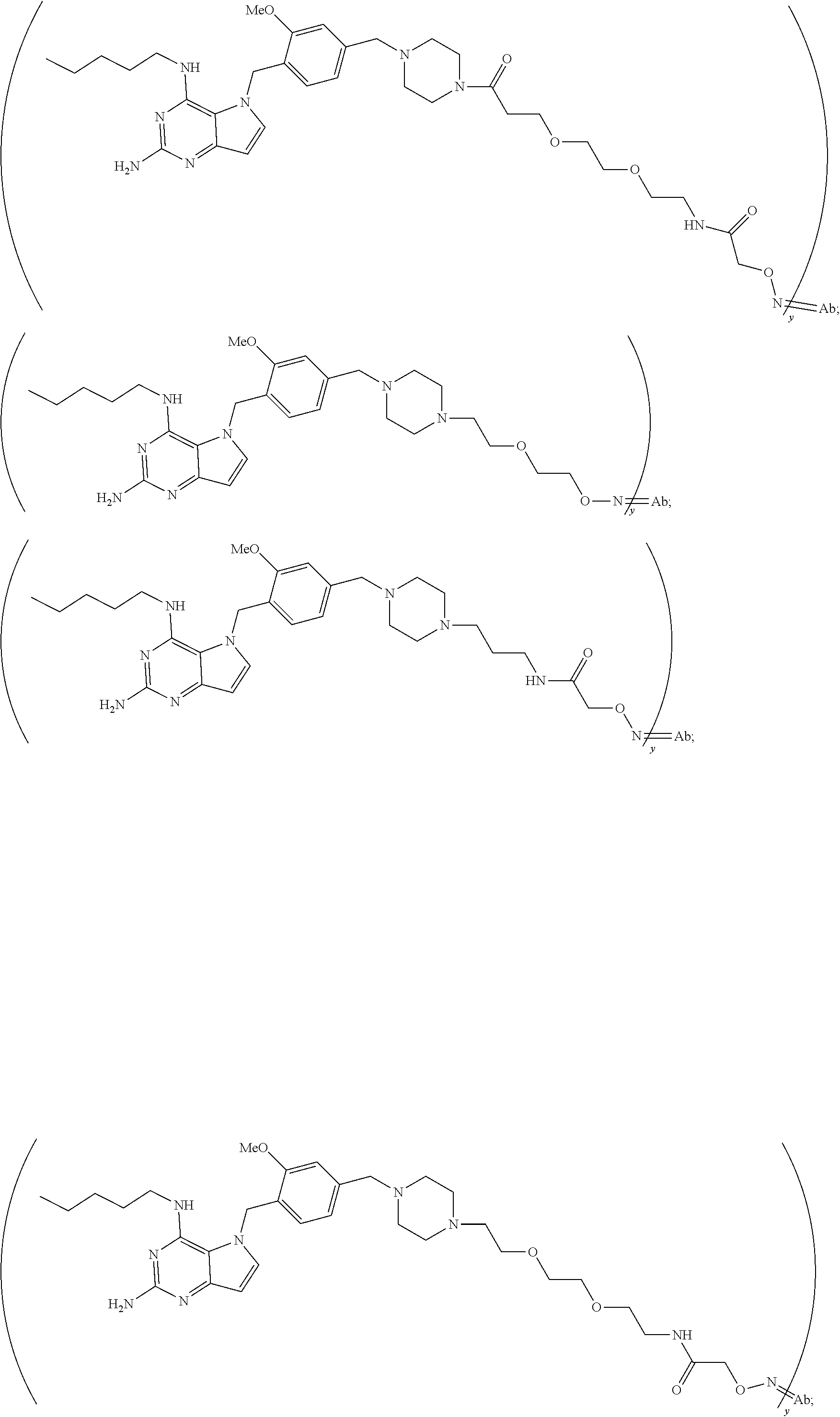 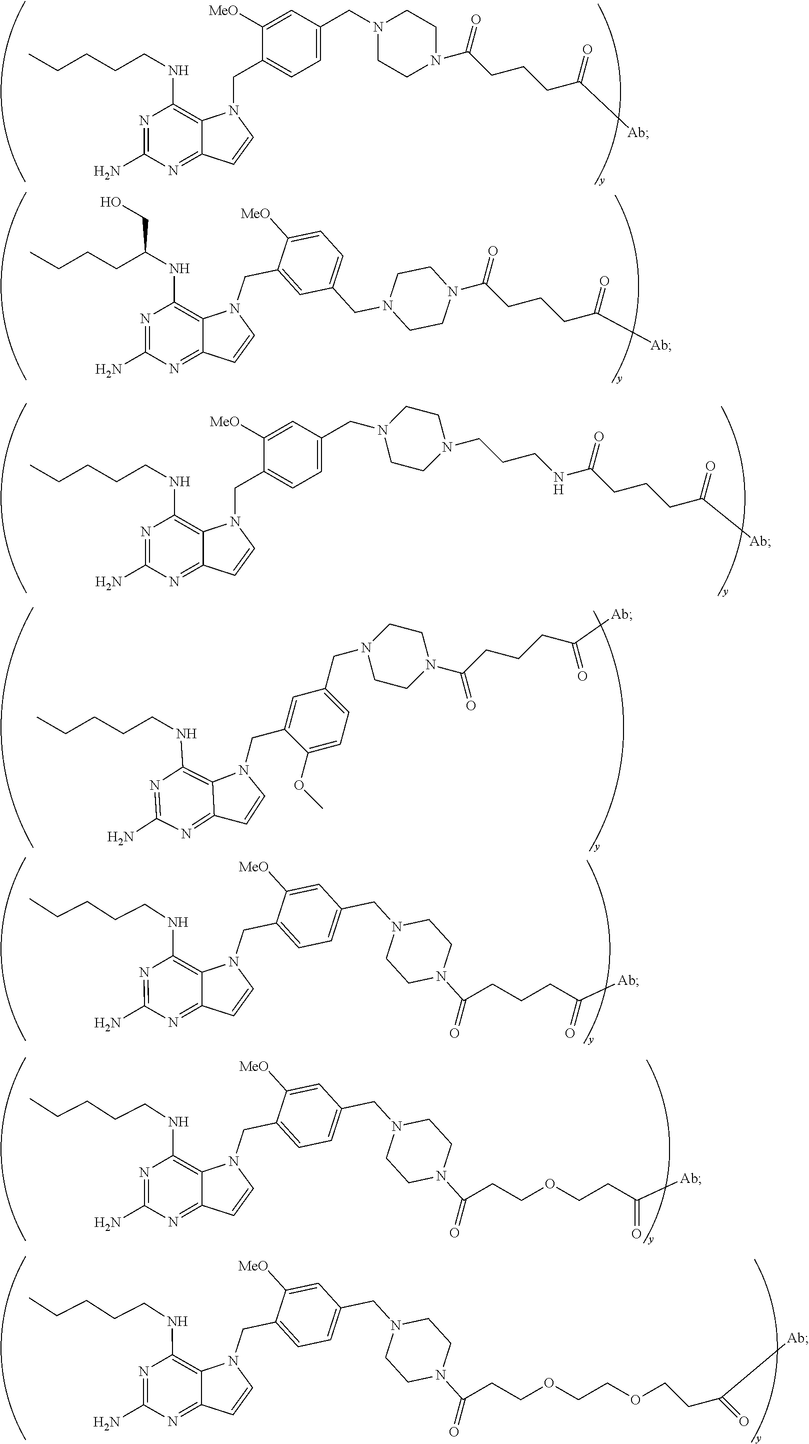 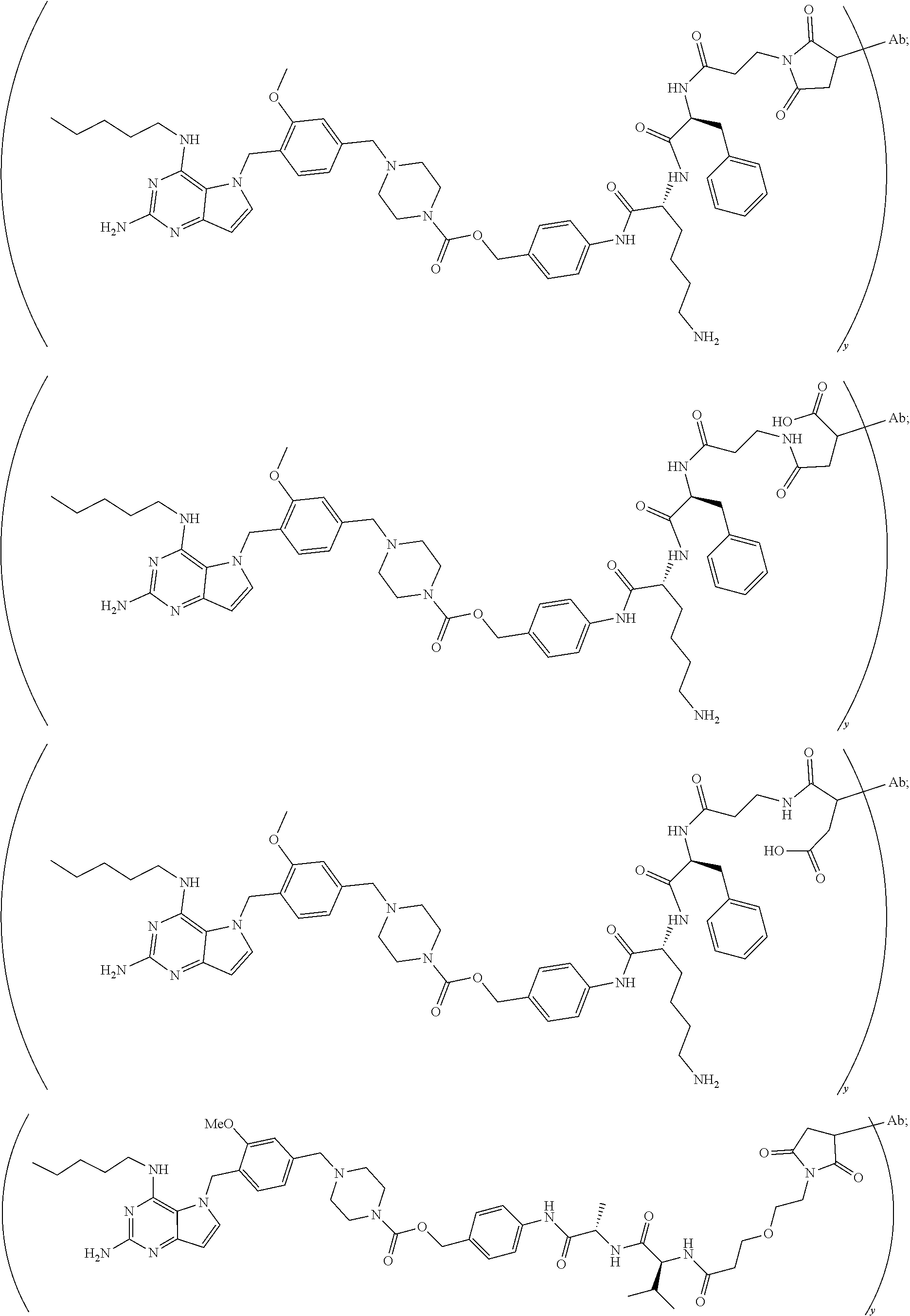 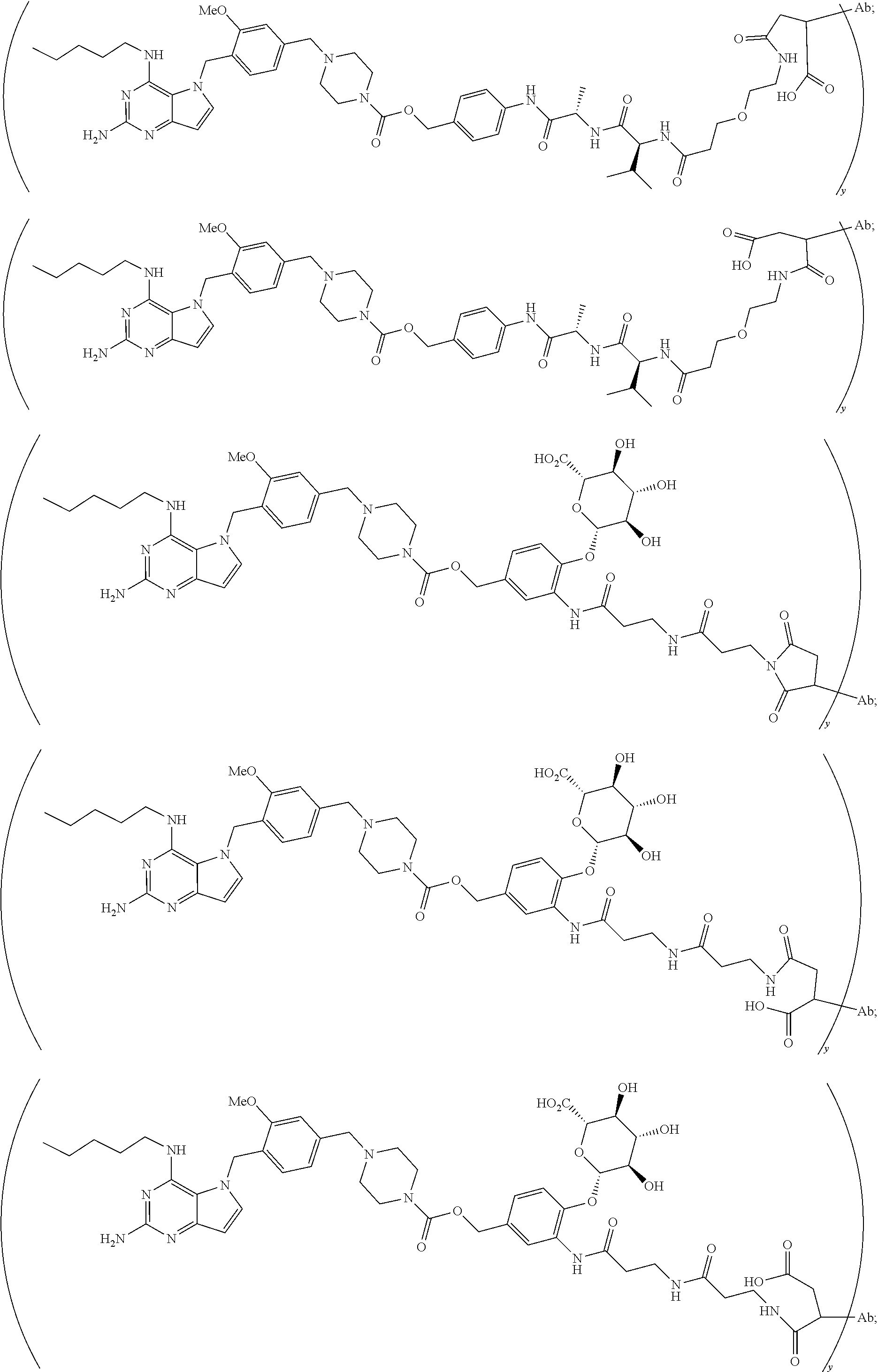 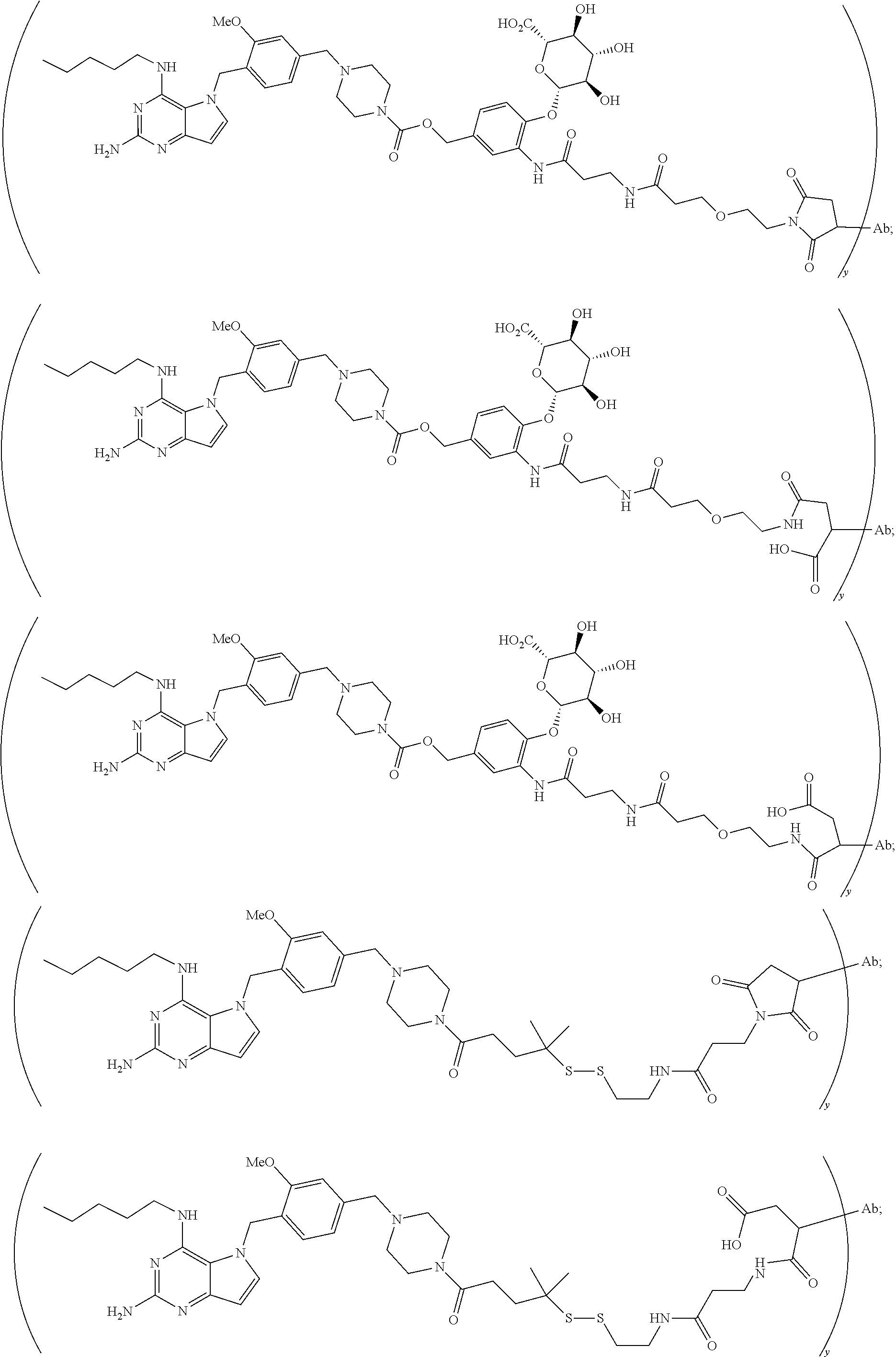 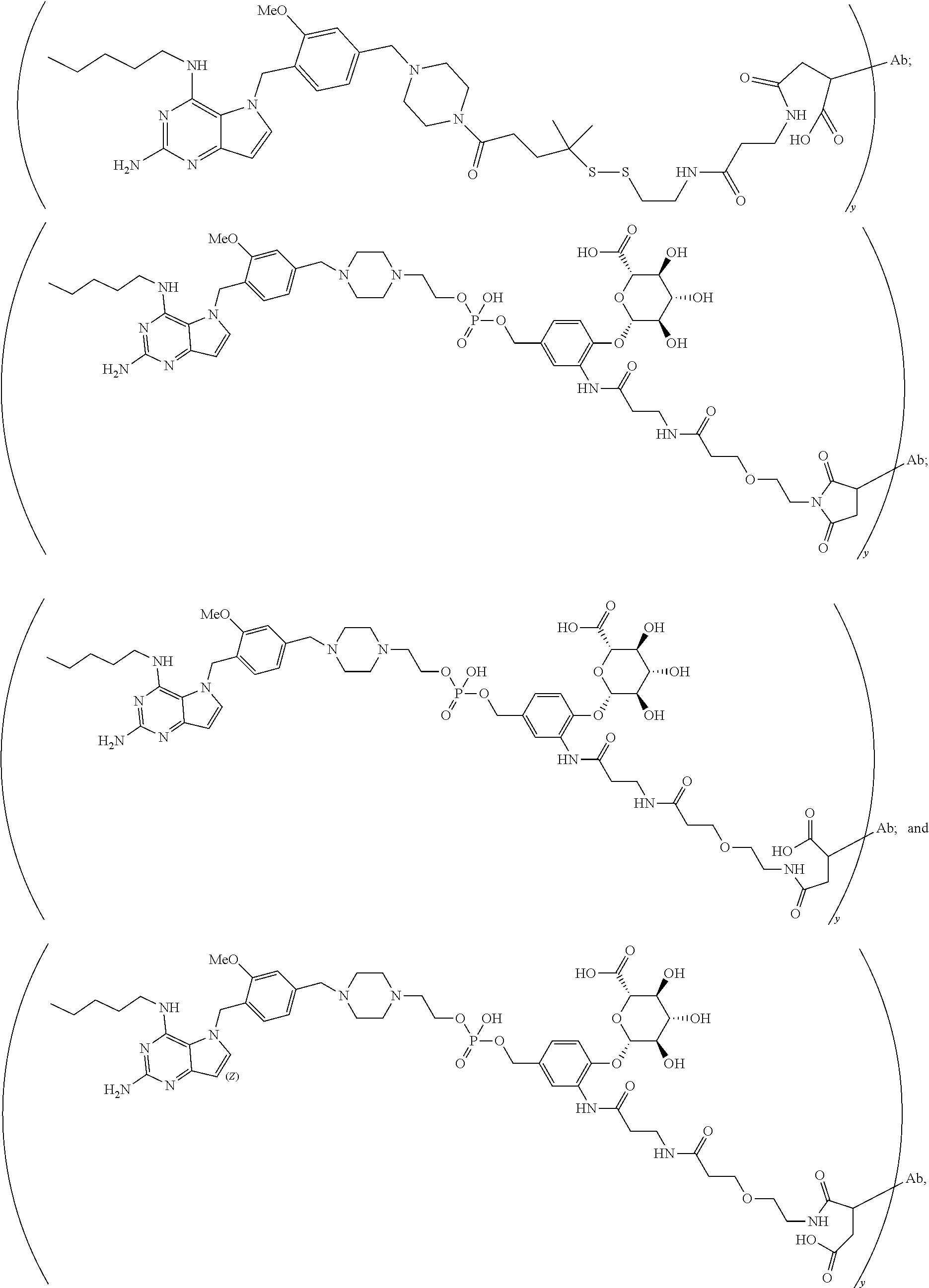
wherein y is an integer from 1 to 4.

30. The conjugate of claim 29, wherein y is about 3 to 4.

31. The conjugate of claim 29, wherein the conjugate has a hydrophobicity index of 0.8 or greater, as determined by hydrophobic interaction chromatography.

32. The conjugate of claim 24 comprising Formula (II):
(RIGIa-L-R⁴⁰)_{y}-Ab Formula (II)
wherein:
RIGIa is a RIG-I agonist selected from:
b)
(SEQ ID NO: 338)
5′ppp-GGACGUACGC(UX_{M}CG)GCGUACGUCC-3′
or
b)
(SEQ ID NO: 339)
5′OH-GGACGUACGC(UX_{M}CG)GCGUACGUCC-3′
where:
ppp-G is 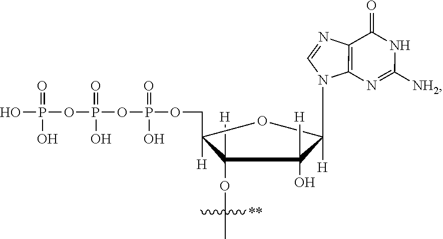
where the ** of ppp-G is the point of attachment toward the 3′ end;
OH-G is 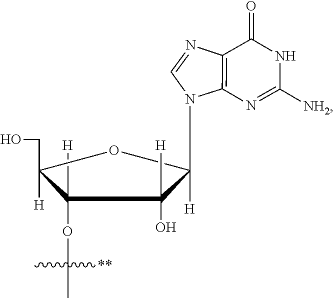
where the ** of OH-G is the point of attachment toward the 3′ end;
G is 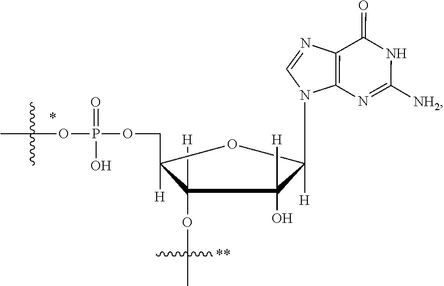
where the * of G is the point of attachment toward the 5′ end and the ** of G is the point of attachment toward the 3′ end;
A is 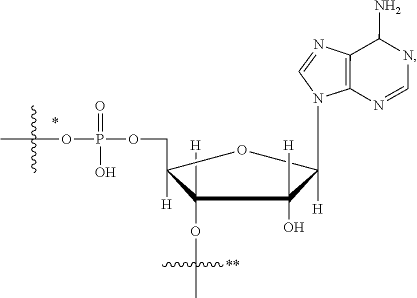
where the * of A is the point of attachment toward the 5′ end and the ** of A is the point of attachment the point of attachment toward the 3′ end;
C is 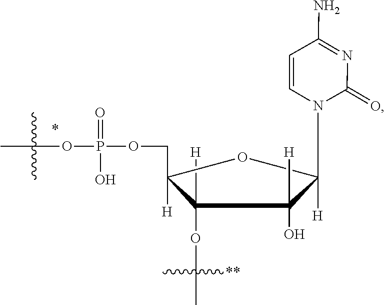
where the * of C is the point of attachment toward the 5′ end and the ** of C is the point of attachment toward the 3′ end;
or if C is in a 3′ terminal position, then C is 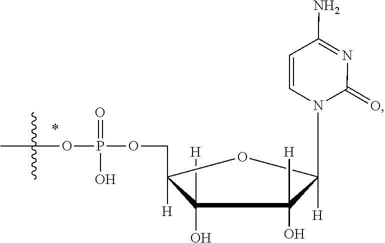
where the * of C is the point of attachment toward the 5′ end;
U is 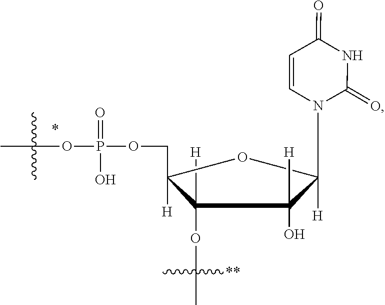
where the * of U is the point of attachment toward the 5′ end and the ** of U is the point of attachment toward the 3′ end;
and
X_{M} is 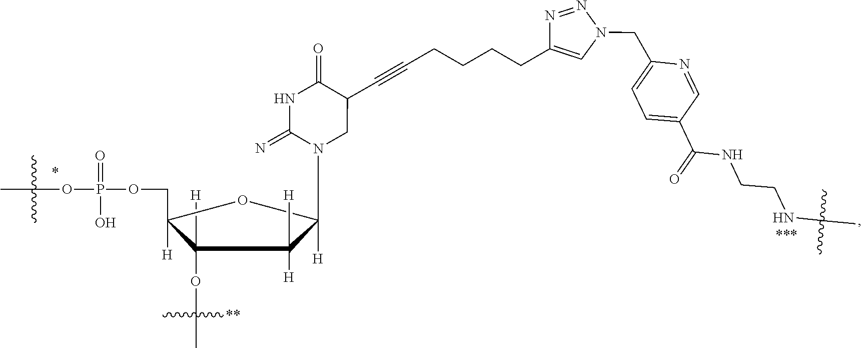
where the * of X_{M} is the point of attachment toward the 5′ end, the * * of X_{M} is the point of attachment toward the 3′ end and the *** of X_{M} is the point of attachment to L;
L is —C(═O)(CH₂)ₙ—**, —(CH₂)ₙ—, —((CH₂)ₙO)ₜ(CH₂)ₙ—**, —C(═O)((CH₂)ₙO)ₜ(CH₂)ₙ—**, —C(═O)X₂X₃C(═O)(CH₂)ₙ—**; —C(═O)X₂X₃((CH₂)ₙO)ₜ(CH₂)ₙ—**, —C(═O)X₂X₃C(═O)(CH₂)ₘO(CH₂)ₘC(═O)—**; —(CH₂)ₙNHC(═O)(CH₂)ₙ—, —(CH₂)ₙNHC(═O)(CH₂)ₙC(═O)NH(CH₂)ₙ—**, —((CH₂)ₙO)ₜ(CH₂)ₙNHC(═O)(CH₂)ₙ—**, —C(═O)((CH₂)ₙO)ₜ(CH₂)ₙX₁(CH₂)ₙ—**, —C(═O)((CH₂)ₙO)ₜ(CH₂)ₙNHC(═O)(CH₂)ₙ—**, —C(═O)((CH₂)ₙO)ₜ(CH₂)ₙC(═O)NH(CH₂)ₙ—**, —C(═O)NH((CH₂)ₙO)ₜ(CH₂)ₙX₁(CH₂)ₙ—**, —C(═O)O(CH₂)ₙC(R⁷)₂SS(CH₂)ₙNHC(═O)(CH₂)ₙ—** or —C(═O)(CH₂)ₙC(═O)NH(CH₂)ₙ—**, where the ** of L indicates the point of attachment to R⁴⁰;
R⁴⁰ is 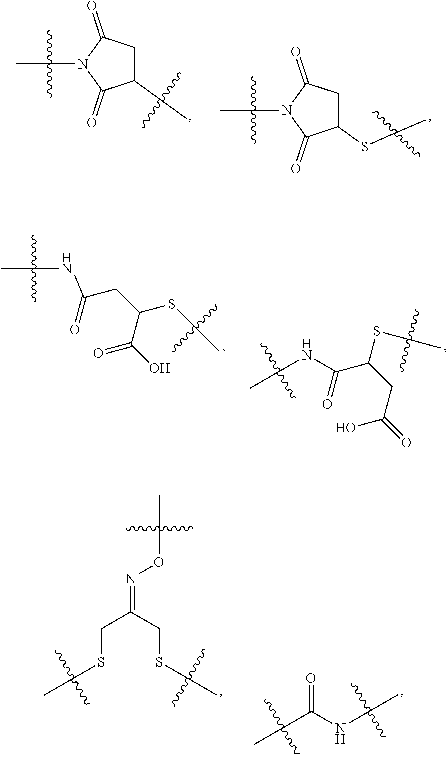 [SMILES: *C1CC(=O)N(*)C1=O ; *SC1CC(=O)N(*)C1=O ; *NC(=O)CC(S*)C(=O)O ; *NC(=O)C(CC(=O)O)S* ; *ON=C(CS*)CS* ; *NC(*)=O]
—S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—, 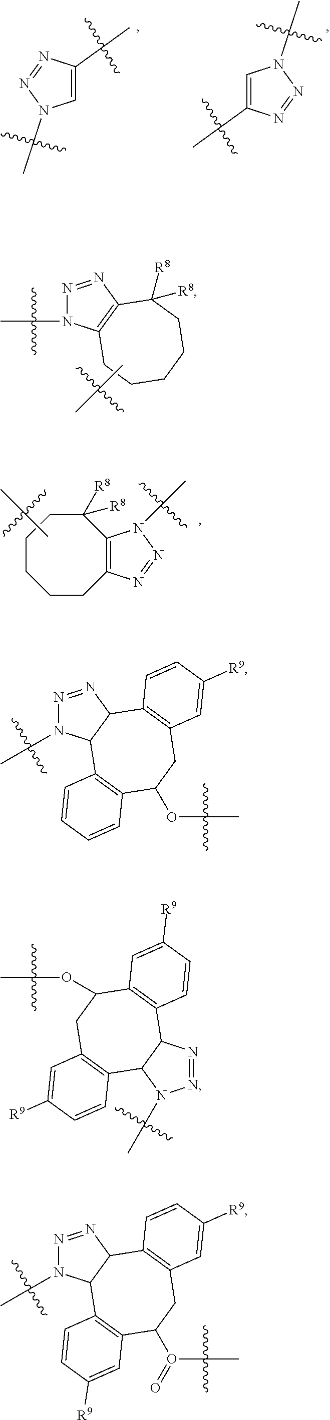 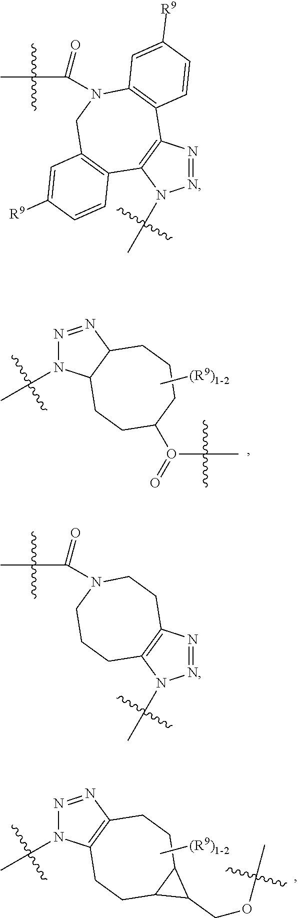 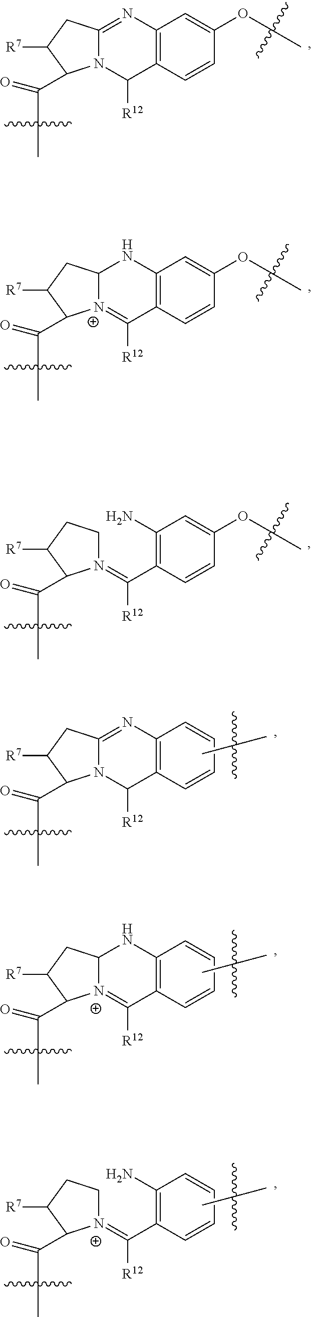 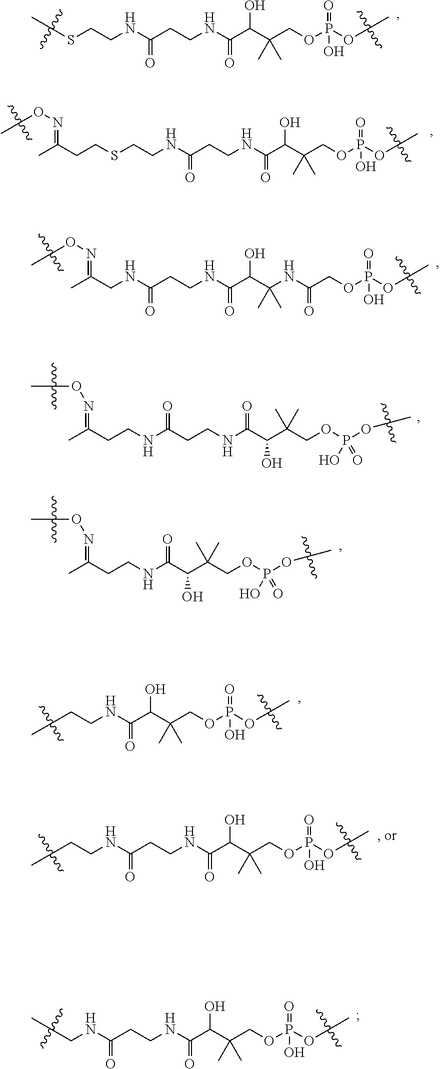
X₁ is 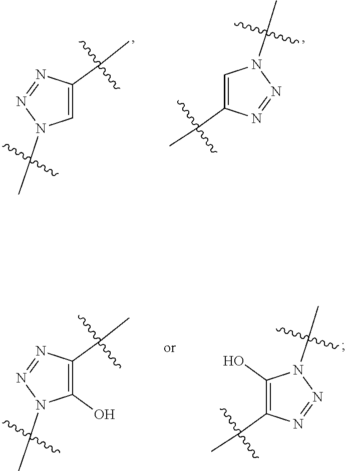 [SMILES: *c1cn(*)nn1 ; *c1cn(*)nn1 ; *c1nnn(*)c1O ; *c1nnn(*)c1O]
X₂ is 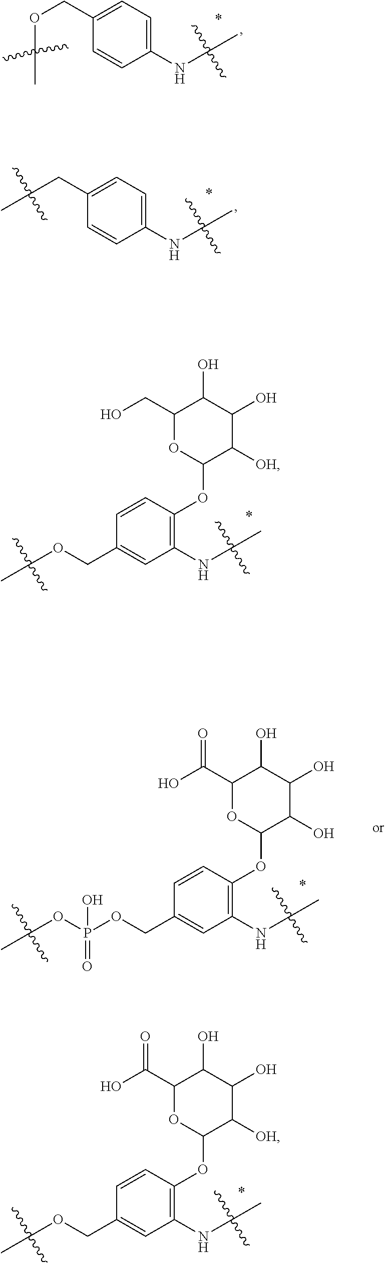 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]
where the * of X₂ indicates the point of attachment to X₃;
X₃ is 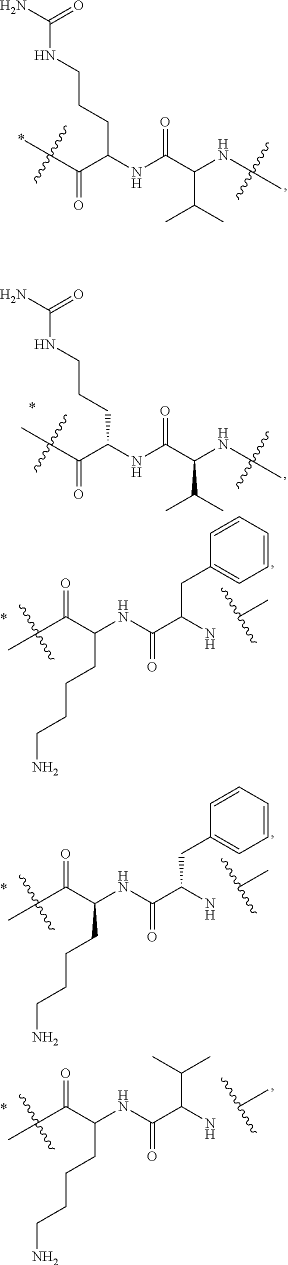 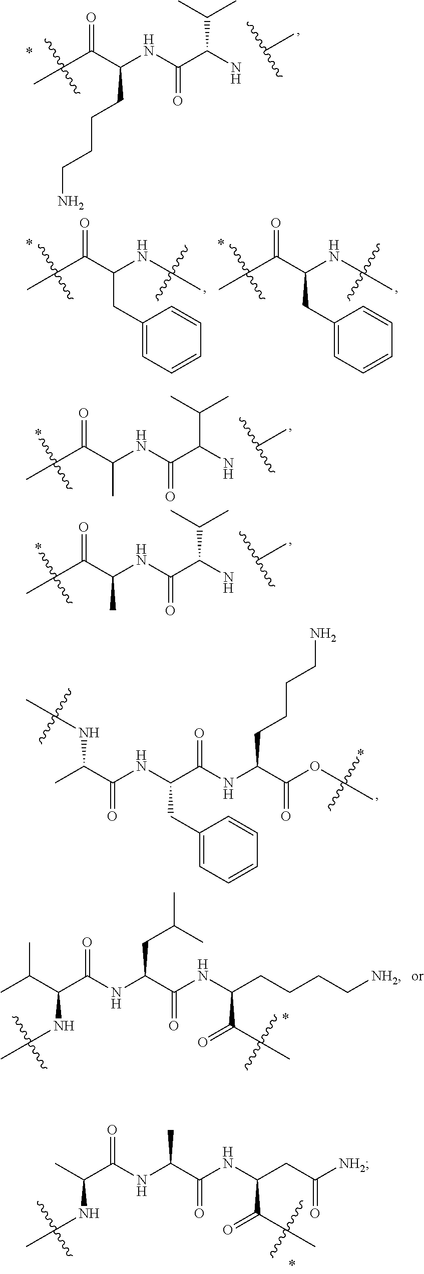
where the * of X₃ indicates the point of attachment to X₂;
each R⁷ is independently selected from H and C₁-C₆alkyl;
each R⁸ is independently selected from H, C₁-C₆alkyl, F, Cl, and —OH;
each R⁹ is independently selected from H, C₁-C₆alkyl, F, Cl, —NH₂, —OCH₃, —OCH₂CH₃, —N(CH₃)₂, —CN, —NO₂ and —OH;
each R¹⁰ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
R¹² is H, methyl or phenyl;
each m is independently selected from 1, 2, 3, and 4;
each n is independently selected from 1, 2, 3, and 4;
each t is independently selected from 1, 2, 3, 4, 5, 6, 7, 8, 9, 10, 11, 12, 13, 14, 15, 16, 17 and 18, and
y is an integer from 1 to 16.

33. A fusion protein comprising the antibody or antigen binding fragment thereof of claim 1 linked to a peptide antigen.

34-36. (canceled)

37. A pharmaceutical composition comprising the antibody or antigen binding fragment thereof of claim 1, one or more conjugates of claim 18, or the fusion protein of claim 33, and a pharmaceutically acceptable carrier.

38. A method of treating a cancer in a subject in need thereof, the method comprising administering to the subject a therapeutically effective amount of the conjugate of claim 18.

39-49. (canceled)

50. A diagnostic reagent comprising the antibody or antigen binding fragment thereof according to claim 1.

51. (canceled)

52. A method of treating an autoimmune disease in a subject in need thereof, the method comprising administering to the subject a therapeutically effective amount of the fusion protein of claim 33.

53. (canceled)
